(19) United States
(12) Patent Application Publication
(10) Pub. No.: US 2021/0393689 A1
(43) Pub. Date: Dec. 23, 2021

(54) CHIMERIC ANTIGEN RECEPTORS SPECIFIC FOR G PROTEIN-COUPLED RECEPTOR CLASS C GROUP 5 MEMBER D (GPRC5D)

(71) Applicants: Juno Therapeutics, Inc., Seattle, WA (US); Memorial Sloan Kettering Cancer Center, New York, NY (US)

(72) Inventors: Blythe D. SATHER, Seattle, WA (US); Eric L. SMITH, New York, NY (US); Cyr DE IMUS, Seattle, WA (US); Kimberly HARRINGTON, Seattle, WA (US); Jon JONES, Seattle, WA (US); Aye CHEN, Seattle, WA (US); Semih TAREEN, Seattle, WA (US); Erik HESS, Seattle, WA (US); Stefan PONKO, Seattle, WA (US); Audrey OLSHEFSKY, Seattle, WA (US); Carlos FERNANDEZ DE LARREA, Barcelona (ES); Renier BRENTJENS, New York, NY (US)

(73) Assignees: Juno Therapeutics, Inc., Seattle, WA (US); Memorial Sloan Kettering Cancer Center, New York, NY (US)

(21) Appl. No.: 17/290,060

(22) PCT Filed: Oct. 31, 2019
(86) PCT No.: PCT/US2019/059285

§ 371 Date: Apr. 29, 2021

Related U.S. Application Data
(60) Provisional application No. 62/754,576, filed on Nov. 1, 2018.
(60) Provisional application No. 62/774,159, filed on Nov. 30, 2018.
(60) Provisional application No. 62/819,422, filed on Mar. 15, 2019.
(60) Provisional application No. 62/904,187, filed on Sep. 23, 2019.
(60) Provisional application No. 62/904,197, filed on Sep. 23, 2019.

(51) Int. Cl. A61K 35/17; C07K 16/28; C07K 16/30; A61P 35/00

(52) U.S. Cl. CPC A61K 35/17; C07K 16/2866; A61K 2039/505; A61P 35/00; C07K 16/3061

(57) ABSTRACT

Provided are chimeric antigen receptors (CARs), which contain antibody portions specific to G Protein-Coupled Receptor Class C Group 5 Member D (GPRC5D) and polynucleotides that encode CARs specific for GPRC5D. The disclosure further relates to genetically engineered cells, containing such GPRCSD-binding receptors, and uses thereof in adoptive cell therapy.

### CROSS-REFERENCE TO RELATED APPLICATIONS

This application claims priority to U.S. provisional applications 62/754,576, filed Nov. 1, 2018, entitled “CHIMERIC ANTIGEN RECEPTORS SPECIFIC FOR G PROTEIN-COUPLED RECEPTOR CLASS C GROUP 5 MEMBER D (GPRC5D)”; 62/774,159, filed Nov. 30, 2018, entitled “CHIMERIC ANTIGEN RECEPTORS SPECIFIC FOR G PROTEIN-COUPLED RECEPTOR CLASS C GROUP 5 MEMBER D (GPRC5D)”; 62/819,422 filed Mar. 15, 2019, entitled “CHIMERIC ANTIGEN RECEPTORS SPECIFIC FOR G PROTEIN-COUPLED RECEPTOR CLASS C GROUP 5 MEMBER D (GPRC5D)”; 62/904,197 filed Sep. 23, 2019, entitled “CHIMERIC ANTIGEN RECEPTORS SPECIFIC FOR G PROTEIN-COUPLED RECEPTOR CLASS C GROUP 5 MEMBER D (GPRC5D)”; and 62/904,187, filed Sep. 23, 2019, entitled “BICISTRONIC POLYNUCLEOTIDE CONSTRUCTS ENCODING CHIMERIC ANTIGEN RECEPTORS,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 735042013740SeqList.TXT, created Oct. 31, 2019, which is 294 kilobytes in size. The information in the electronic format of the Sequence Listing is incorporated by reference in its entirety.

### FIELD

The present disclosure relates in some aspects to chimeric antigen receptors (CARs), which contain antibody portions specific to G Protein-Coupled Receptor Class C Group 5 Member D (GPRC5D) and polynucleotides that encode CARs specific for GPRC5D. The disclosure further relates to genetically engineered cells, containing such GPRC5D-binding receptors, and uses thereof in adoptive cell therapy.

### BACKGROUND

G-protein coupled receptor class C group 5 member D (GPRC5D) is a G-protein coupled receptor, the specific function of which has not yet been determined. The expression of GPRC5D is high in bone marrow samples of patients with multiple myeloma (MM) compared to the minimal expression of GPRC5D in bone marrow samples of patients with other hematological malignancies. Based on its expression, GPRC5D could be a marker of MM tumors and a therapeutic target. Various GPRC5D-binding chimeric antigen receptors (CARs), and cells expressing such CARs, are available. However, there remains a need for improved GPRC5D-binding CARs and engineered GPRC5D-CAR expressing targeting cells, such as for use in adoptive cell therapy. Provided herein are embodiments that meet such needs.

### SUMMARY

Provided herein are chimeric antigen receptors containing: (1) an extracellular antigen-binding domain that specifically binds human G-protein coupled receptor class C group 5 member D (GPRC5D), wherein the extracellular antigen-binding domain contains: (i) a variable heavy chain (V_{H}) region containing an amino acid sequence having at least 90%, 91%, 92%, 93%, 94%, 95%, 96%, 97%, 98%, or 99% sequence identity to the V_{H} region amino acid sequence set forth in any of SEQ ID NO:21, 23, 25, 27, 29, 31 or 33; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO:22, 24, 26, 28, 30, 32 or 34; (2) a spacer of at least 125 amino acids in length; (3) a transmembrane domain; and (4) an intracellular signaling region.

Also provided are chimeric antigen receptors containing: (1) an extracellular antigen-binding domain that specifically binds human G-protein coupled receptor class C group 5 member D (GPRC5D), wherein the extracellular antigen-binding domain contains: (i) a variable heavy chain (V_{H}) containing a CDR-H1, CDR-H2 and CDR-H3 contained within the V_{H} region amino acid sequence selected from any one of SEQ ID NOs: 21, 23, 25, 27, 29, 31 or 33; and (ii) a variable light chain (V_{L}) region containing a CDR-L1, CDR-L2 and CDR-L3 contained within the V_{L} region amino acid sequence selected from any one of SEQ ID NOs: 22, 24, 26, 28, 30, 32 or 34; (2) a spacer of at least 125 amino acids in length; (3) a transmembrane domain; and (4) an intracellular signaling region.

Also provided are chimeric antigen receptors containing: (1) an extracellular antigen-binding domain that specifically binds human G-protein coupled receptor class C group 5 member D (GPRC5D) wherein the extracellular antigen-binding domain contains: (i) a variable heavy chain (V_{H}) containing a heavy chain complementarity determining region 1 (CDR-H1) containing the amino acid sequence selected from any one of SEQ ID NOs: 75, 78, 80, 82, 90, 93, 95, 97, 105, 108, 110, 112, 120, 123, 125, 127, 135, 138, 140, 142, 135, 152, 162, 165, 167 or 169; (b) a heavy chain complementarity determining region 2 (CDR-H2) containing the amino acid sequence selected from any one of SEQ ID NOs: 76, 79, 81, 83, 91, 94, 96, 98, 106, 109, 111, 113, 121, 124, 126, 128, 136, 139, 141, 143, 150, 153, 154, 155, 163, 166, 169 or 170; and (c) a heavy chain complementarity determining region 3 (CDR-H3) containing the amino acid sequence selected from any one of SEQ ID NOs: 77, 84, 92, 99, 107, 114, 133, 129, 137, 144, 151, 156, 164 or 171; and (ii) a variable light chain (V_{L}) region containing a light chain complementarity determining region 1 (CDR-L1) containing the amino acid sequence selected from any one of SEQ ID NOs: 85, 88, 100, 103, 115, 118, 130, 133, 145, 148, 157, 160, 172 or 174; (b) a light chain complementarity determining region 2 (CDR-L2) containing the amino acid sequence selected from any one of SEQ ID NOs: 86, 89, 101, 104, 116, 119, 131, 134, 146, 149, 158 or 161; and (c) a light chain complementarity determining region 3 (CDR-L3) containing the amino acid sequence selected from any one of SEQ ID NOs: 87, 102, 117, 132, 147, 159, 173, 175, or 297; (2) a spacer of at least 125 amino acids in length; (3) a transmembrane domain; and (4) an intracellular signaling region.

In some of any of the provided embodiments, the extracellular antigen-binding domain of the chimeric antigen receptor contains: (i) a variable heavy chain (V_{H}) containing: an amino acid sequence having at least 90%, 91%, 92%, 93%, 94%, 95%, 96%, 97%, 98%, or 99% sequence identity to the V_{H} region amino acid sequence set forth in any of SEQ ID NO:21, 23, 25, 27, 29, 31 or 33; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO:22, 24, 26, 28, 30, 32 or 34.

In some of any of the provided embodiments, the spacer has a length from or from about 125 to 300 amino acids in length, 125 to 250 amino acids in length, 125 to 230 amino acids in length, 125 to 200 amino acids in length, 125 to 180 amino acids in length, 125 to 150 amino acids in length, 150 to 300 amino acids in length, 150 to 250 amino acids in length, 150 to 230 amino acids in length, 150 to 200 amino acids in length, 150 to 180 amino acids in length, 180 to 300 amino acids in length, 180 to 250 amino acids in length, 180 to 230 amino acids in length, 180 to 200 amino acids in length, 200 to 300 amino acids in length, 200 to 250 amino acids in length, 200 to 230 amino acids in length, 230 to 300 amino acids in length, 230 to 250 amino acids in length or 250 to 300 amino acids in length. In some of any of the provided embodiments, the spacer is at least or at least about or is or is about 130, 140, 150, 160, 170, 180, 190, 200, 210, 220, 221, 222, 223, 224, 225, 226, 227, 228 or 229 amino acids in length, or has a length between any of the foregoing.

In some of any of the provided embodiments, the spacer is derived from an immunoglobulin. In some of any of the provided embodiments, the spacer contains a sequence of a hinge region, a CH2 and CH3 region. In some of any of the provided embodiments, one of more of the hinge, CH2 and CH3 is derived all or in part from IgG4 or IgG2, optionally human IgG4 or human IgG2. In some of any of the provided embodiments, the hinge, CH2 and CH3 is derived from IgG4. In some of any of the provided embodiments, one or more of the hinge, CH2 and CH3 is chimeric and contains a sequence derived from IgG4 and IgG2. In some of any of the provided embodiments, the spacer contains an IgG4/2 chimeric hinge or a modified IgG4 containing at least one amino acid replacement compared to human IgG4, an IgG2/4 chimeric CH2, and an IgG4 CH3 region.

In some of any of the provided embodiments, the spacer is or contains (i) the sequence set forth in SEQ ID NO: 17; (ii) a functional variant of SEQ ID NO:17 that has at least 95%, 96%, 97%, 98% or 99% sequence identity to SEQ ID NO:17; or (iii) a contiguous portion of (i) or (ii) that is at least 125 amino acids in length. In some of any of the provided embodiments, the spacer is or contains the sequence set forth in SEQ ID NO:17. In some of any of the provided embodiments, the spacer is or contains a sequence encoded by the sequence of nucleotides set forth in SEQ ID NO:48 (also set forth in SEQ ID NO:74).

In some of any of the provided embodiments, the V_{H} region and the V_{L} regions contain the amino acid sequence set forth in SEQ ID NOs:21 and 22, respectively, or a sequence of amino acids having at least 90%, 91%, 92%, 93%, 94%, 95%, 96%, 97%, 98%, or 99% identity to SEQ ID NOS:21 and 22, respectively; the V_{H} region and the V_{L} regions contain the amino acid sequence set forth in SEQ ID NOs:23 and 24, respectively, or a sequence of amino acids having at least 90%, 91%, 92%, 93%, 94%, 95%, 96%, 97%, 98%, or 99% identity to SEQ ID NOS:23 and 24, respectively; the V_{H} region and the V_{L} regions contain the amino acid sequence set forth in SEQ ID NOs:25 and 26, respectively, or a sequence of amino acids having at least 90%, 91%, 92%, 93%, 94%, 95%, 96%, 97%, 98%, or 99% identity to SEQ ID NOS:25 and 26, respectively; the V_{H} region and the V_{L} regions contain the amino acid sequence set forth in SEQ ID NOs:27 and 28, respectively, or a sequence of amino acids having at least 90%, 91%, 92%, 93%, 94%, 95%, 96%, 97%, 98%, or 99% identity to SEQ ID NOS:27 and 28, respectively; the V_{H} region and the V_{L} regions contain the amino acid sequence set forth in SEQ ID NOs:29 and 30, respectively, or a sequence of amino acids having at least 90%, 91%, 92%, 93%, 94%, 95%, 96%, 97%, 98%, or 99% identity to SEQ ID NOS:29 and 30, respectively; the V_{H} region and the V_{L} regions contain the amino acid sequence set forth in SEQ ID NOs:31 and 32, respectively, or a sequence of amino acids having at least 90%, 91%, 92%, 93%, 94%, 95%, 96%, 97%, 98%, or 99% identity to SEQ ID NOS:31 and 32, respectively; or the V_{H} region and the V_{L} regions contain the amino acid sequence set forth in SEQ ID NOs:33 and 34, respectively, or a sequence of amino acids having at least 90%, 91%, 92%, 93%, 94%, 95%, 96%, 97%, 98%, or 99% identity to SEQ ID NOS:33 and 34, respectively.

In some of any of the provided embodiments, the V_{H} region contains a CDR-H1, CDR-H2, and CDR-H3 containing the amino acid sequence of SEQ ID NOS:80, 81 and 77, respectively, and the V_{L} region contains a CDR-L1, CDR-L2, and CDR-L3 containing the amino acid sequence of SEQ ID NOS:85, 86 and 87, respectively; the V_{H} region contains a CDR-H1, CDR-H2, and CDR-H3 containing the amino acid sequence of SEQ ID NOS:82, 83 and 84, respectively, and the V_{L} region contains a CDR-L1, CDR-L2, and CDR-L3 containing the amino acid sequence of SEQ ID NOS:88, 89 and 87, respectively; the V_{H} region contains a CDR-H1, CDR-H2, and CDR-H3 containing the amino acid sequence of SEQ ID NOS:95, 96, 92, respectively, and the V_{L} region contains a CDR-L1, CDR-L2, and CDR-L3 containing the amino acid sequence of SEQ ID NOS:100, 101 and 102, respectively; the V_{H} region contains a CDR-H1, CDR-H2, and CDR-H3 containing the amino acid sequence of SEQ ID NOS:97, 98 and 99, respectively, and the V_{L} region contains a CDR-L1, CDR-L2, and CDR-L3 containing the amino acid sequence of SEQ ID NOS:103, 104 and 102, respectively; the V_{H} region contains a CDR-H1, CDR-H2, and CDR-H3 containing the amino acid sequence of SEQ ID NOS:110, 111 and 107, respectively, and the V_{L} region contains a CDR-L1, CDR-L2, and CDR-L3 containing the amino acid sequence of SEQ ID NOS:115, 116 and 117, respectively; the V_{H} region contains a CDR-H1, CDR-H2, and CDR-H3 containing the amino acid sequence of SEQ ID NOS:112, 113 and 114, respectively, and the V_{L} region contains a CDR-L1, CDR-L2, and CDR-L3 containing the amino acid sequence of SEQ ID NOS:118, 119 and 117, respectively; the V_{H} region contains a CDR-H1, CDR-H2, and CDR-H3 containing the amino acid sequence of SEQ ID NOS:125, 126 and 122, respectively, and the V_{L} region contains a CDR-L1, CDR-L2, and CDR-L3 containing the amino acid sequence of SEQ ID NOS:130, 131 and 132, respectively; the V_{H} region contains a CDR-H1, CDR-H2, and CDR-H3 containing the amino acid sequence of SEQ ID NOS:127, 128 and 129, respectively, and the V_{L} region contains a CDR-L1, CDR-L2, and CDR-L3 containing the amino acid sequence of SEQ ID NOS:133, 134 and 132, respectively; the V_{H} region contains a CDR-H1, CDR-H2, and CDR-H3 containing the amino acid sequence of SEQ ID NOS:140, 141 and 137, respectively, and the V_{L} region contains a CDR-L1, CDR-L2, and CDR-L3 containing the amino acid sequence of SEQ ID NOS:145, 146 and 147, respectively; the V_{H} region contains a CDR-H1, CDR-H2, and CDR-H3 containing the amino acid sequence of SEQ ID NOS:142, 143 and 144, respectively, and the V_{L} region contains a CDR-L1, CDR-L2, and CDR-L3 containing the amino acid sequence of SEQ ID NOS:148, 149 and 147, respectively; the V_{H} region contains a CDR-H1, CDR-H2, and CDR-H3 containing the amino acid sequence of SEQ ID NOS:140, 154 and 151, respectively, and the V_{L} region contains a CDR-L1, CDR-L2, and CDR-L3 containing the amino acid sequence of SEQ ID NOS:157, 158 and 159, respectively; the V_{H} region contains a CDR-H1, CDR-H2, and CDR-H3 containing the amino acid sequence of SEQ ID NOS:142, 155 and 156, respectively, and the V_{L} region contains a CDR-L1, CDR-L2, and CDR-L3 containing the amino acid sequence of SEQ ID NOS:160, 161 and 159, respectively; the V_{H} region contains a CDR-H1, CDR-H2, and CDR-H3 containing the amino acid sequence of SEQ ID NOS:167, 168 and 164, respectively, and the V_{L} region contains a CDR-L1, CDR-L2, and CDR-L3 containing the amino acid sequence of SEQ ID NOS:172, 86, 173, respectively; the V_{H} region contains a CDR-H1, CDR-H2, and CDR-H3 containing the amino acid sequence of SEQ ID NOS:169, 170 and 171, respectively, and the V_{L} region contains a CDR-L1, CDR-L2, and CDR-L3 containing the amino acid sequence of SEQ ID NOS:174, 89 and 175, respectively; or the V_{H} region contains a CDR-H1, CDR-H2, and CDR-H3 containing the amino acid sequence of SEQ ID NOS:169, 170 and 171, respectively, and the V_{L} region contains a CDR-L1, CDR-L2, and CDR-L3 containing the amino acid sequence of SEQ ID NOS:174, 89 and 297, respectively.

In some of any of the provided embodiments, the V_{H} region and the V_{L} regions contain the amino acid sequence set forth in SEQ ID NOs:21 and 22, respectively; the V_{H} region and the V_{L} regions contain the amino acid sequence set forth in SEQ ID NOs:23 and 24, respectively; the V_{H} region and the V_{L} regions contain the amino acid sequence set forth in SEQ ID NOs:25 and 26, respectively; the V_{H} region and the V_{L} regions contain the amino acid sequence set forth in SEQ ID NOs:27 and 28, respectively; the V_{H} region and the V_{L} regions contain the amino acid sequence set forth in SEQ ID NOs:29 and 30, respectively; the V_{H} region and the V_{L} regions contain the amino acid sequence set forth in SEQ ID NOs:31 and 32, respectively; or the V_{H} region and the V_{L} regions contain the amino acid sequence set forth in SEQ ID NOs:33 and 34, respectively.

In some of any of the provided embodiments, the V_{H} region and the V_{L} regions contain the amino acid sequence set forth in SEQ ID NOs:21 and 22, respectively, or a sequence of amino acids having at least 90%, 91%, 92%, 93%, 94%, 95%, 96%, 97%, 98%, or 99% identity to SEQ ID NOS:21 and 22, respectively; the V_{H} region and the V_{L} regions contain the amino acid sequence set forth in SEQ ID NOs:23 and 24, respectively, or a sequence of amino acids having at least 90%, 91%, 92%, 93%, 94%, 95%, 96%, 97%, 98%, or 99% identity to SEQ ID NOS:23 and 24, respectively; the V_{H} region and the V_{L} regions contain the amino acid sequence set forth in SEQ ID NOs:27 and 28, respectively, or a sequence of amino acids having at least 90%, 91%, 92%, 93%, 94%, 95%, 96%, 97%, 98%, or 99% identity to SEQ ID NOS:27 and 28, respectively; or the V_{H} region and the V_{L} regions contain the amino acid sequence set forth in SEQ ID NOs:31 and 32, respectively, or a sequence of amino acids having at least 90%, 91%, 92%, 93%, 94%, 95%, 96%, 97%, 98%, or 99% identity to SEQ ID NOS:31 and 32, respectively.

In some of any of the provided embodiments, the V_{H} region contains a CDR-H1, CDR-H2, and CDR-H3 containing the amino acid sequence of SEQ ID NOS:80, 81 and 77, respectively, and the V_{L} region contains a CDR-L1, CDR-L2, and CDR-L3 containing the amino acid sequence of SEQ ID NOS:85, 86 and 87, respectively; the V_{H} region contains a CDR-H1, CDR-H2, and CDR-H3 containing the amino acid sequence of SEQ ID NOS:82, 83 and 84, respectively, and the V_{L} region contains a CDR-L1, CDR-L2, and CDR-L3 containing the amino acid sequence of SEQ ID NOS:88, 89 and 87, respectively; the V_{H} region contains a CDR-H1, CDR-H2, and CDR-H3 containing the amino acid sequence of SEQ ID NOS:95, 96, 92, respectively, and the V_{L} region contains a CDR-L1, CDR-L2, and CDR-L3 containing the amino acid sequence of SEQ ID NOS:100, 101 and 102, respectively; the V_{H} region contains a CDR-H1, CDR-H2, and CDR-H3 containing the amino acid sequence of SEQ ID NOS:97, 98 and 99, respectively, and the V_{L} region contains a CDR-L1, CDR-L2, and CDR-L3 containing the amino acid sequence of SEQ ID NOS:103, 104 and 102, respectively; the V_{H} region contains a CDR-H1, CDR-H2, and CDR-H3 containing the amino acid sequence of SEQ ID NOS:125, 126 and 122, respectively, and the V_{L} region contains a CDR-L1, CDR-L2, and CDR-L3 containing the amino acid sequence of SEQ ID NOS:130, 131 and 132, respectively; the V_{H} region contains a CDR-H1, CDR-H2, and CDR-H3 containing the amino acid sequence of SEQ ID NOS:127, 128 and 129, respectively, and the V_{L} region contains a CDR-L1, CDR-L2, and CDR-L3 containing the amino acid sequence of SEQ ID NOS:133, 134 and 132, respectively; the V_{H} region contains a CDR-H1, CDR-H2, and CDR-H3 containing the amino acid sequence of SEQ ID NOS:140, 154 and 151, respectively, and the V_{L} region contains a CDR-L1, CDR-L2, and CDR-L3 containing the amino acid sequence of SEQ ID NOS:157, 158 and 159, respectively; or the V_{H} region contains a CDR-H1, CDR-H2, and CDR-H3 containing the amino acid sequence of SEQ ID NOS:142, 155 and 156, respectively, and the V_{L} region contains a CDR-L1, CDR-L2, and CDR-L3 containing the amino acid sequence of SEQ ID NOS:160, 161 and 159, respectively.

In some of any of the provided embodiments, the V_{H} region and the V_{L} regions contain the amino acid sequence set forth in SEQ ID NOs:21 and 22, respectively; the V_{H} region and the V_{L} regions contain the amino acid sequence set forth in SEQ ID NOs:23 and 24, respectively; the V_{H} region and the V_{L} regions contain the amino acid sequence set forth in SEQ ID NOs:27 and 28, respectively; or the V_{H} region and the V_{L} regions contain the amino acid sequence set forth in SEQ ID NOs:31 and 32, respectively.

In some of any of the provided embodiments, the extracellular antigen-binding domain is cross-reactive or binds mouse GPRC5D and/or is cross-reactive or binds cynomolgus GPRC5D. In some of any of the provided embodiments, the extracellular antigen-binding domain is not cross-reactive to or does not bind mouse GPRC5D or cynomolgus GPRC5D.

In some of any of the provided embodiments, the V_{H} region and the V_{L} region contain the amino acid sequences set forth in SEQ ID NOs:27 and 28, respectively, or a sequence of amino acids having at least 90%, 91%, 92%, 93%, 94%, 95%, 96%, 97%, 98%, or 99% identity to SEQ ID NOS:27 and 28, respectively.

In some of any of the provided embodiments, the chimeric antigen receptor contains a variable heavy chain (V_{H}) containing a CDR-H1, CDR-H2 and CDR-H3 contained within the V_{H} region amino acid sequence set forth in SEQ ID NO: 27; and a variable light chain (V_{L}) region containing a CDR-L1, CDR-L2 and CDR-L3 contained within the V_{L} region amino acid sequence set forth in SEQ ID NO: 28. In some of any of the provided embodiments, the V_{H} region contains a CDR-H1, CDR-H2, and CDR-H3 containing the amino acid sequence of SEQ ID NOS:125, 126 and 122, respectively, and the V_{L} region contains a CDR-L1, CDR-L2, and CDR-L3 containing the amino acid sequence of SEQ ID NOS:130, 131 and 132, respectively. In some of any of the provided embodiments, the V_{H} region contains a CDR-H1, CDR-H2, and CDR-H3 containing the amino acid sequence of SEQ ID NOS:127, 128 and 129, respectively, and the V_{L} region contains a CDR-L1, CDR-L2, and CDR-L3 containing the amino acid sequence of SEQ ID NOS:133, 134 and 132, respectively. In some of any of the provided embodiments, the V_{H} region contains a CDR-H1, CDR-H2, and CDR-H3 containing the amino acid sequence of SEQ ID NOS:120, 121 and 122, respectively, and the V_{L} region contains a CDR-L1, CDR-L2, and CDR-L3 containing the amino acid sequence of SEQ ID NOS:130, 131 and 132, respectively. In some of any of the provided embodiments, the V_{H} region contains a CDR-H1, CDR-H2, and CDR-H3 containing the amino acid sequence of SEQ ID NOS:123, 124 and 122, respectively, and the V_{L} region contains a CDR-L1, CDR-L2, and CDR-L3 containing the amino acid sequence of SEQ ID NOS:130, 131 and 132, respectively. In some of any of the provided embodiments, the V_{H} region and the V_{L} regions contain the amino acid sequence set forth in SEQ ID NOs:27 and 28, respectively.

In some of any of the provided embodiments, the extracellular antigen-binding domain is a single chain antibody fragment. In some of any of the provided embodiments, the fragment is or contains a single chain variable fragment (scFv).

In some of any of the provided embodiments, the V_{H} region and the V_{L} region are joined by a flexible linker. In some of any of the provided embodiments, the V_{H} region and the V_{L} region are joined by a linker containing the amino acid sequence GGGGSGGGGSGGGGS (SEQ ID NO:52). In some of any of the provided embodiments, the V_{H} region and the V_{L} region are joined by a flexible linker. In some of any of the provided embodiments, the V_{H} region and the V_{L} region are joined by a linker containing the amino acid sequence GGGGSGGGGSGGGGSGGGGS (SEQ ID NO:320).

In some of any of the provided embodiments, the V_{H} region is amino-terminal to the V_{L} region. In some of any of the provided embodiments, the antigen-binding domain contains the amino acid sequence selected from any one of SEQ ID NOs: 1, 3, 5, 7, 9, 11 or 13 or an amino acid sequence having at least 90%, 91%, 92%, 93%, 94%, 95%, 96%, 97%, 98%, or 99% sequence identity to the amino acid sequence selected from any one of SEQ ID NOs: 1, 3, 5, 7, 9, 11 or 13. In some of any of the provided embodiments, the antigen-binding domain contains the amino acid sequence selected from any one of SEQ ID NOs: 1, 3, 5, 7, 9, 11 or 13. In some of any of the provided embodiments, the antigen-binding domain contains the amino acid sequence selected from any one of SEQ ID NOs: 1, 3, 7 or 11 or an amino acid sequence having at least 90%, 91%, 92%, 93%, 94%, 95%, 96%, 97%, 98%, or 99% sequence identity to the amino acid sequence selected from any one of SEQ ID NOs: 1, 3, 7 or 11. In some of any of the provided embodiments, the antigen-binding domain contains the amino acid sequence selected from any one of SEQ ID NOs: 1, 3, 7 or 11. In some of any of the provided embodiments, the antigen-binding domain contains the amino acid sequence set forth in SEQ ID NO: 7 or an amino acid sequence having at least 90%, 91%, 92%, 93%, 94%, 95%, 96%, 97%, 98%, or 99% sequence identity to the amino acid sequence set forth in SEQ ID NO: 7. In some of any of the provided embodiments, the antigen-binding domain contains the amino acid sequence set forth in SEQ ID NO: 7.

In some of any of the provided embodiments, the V_{H} region is carboxy-terminal to the V_{L} region. In some of any of the provided embodiments, the antigen-binding domain contains the amino acid sequence selected from any one of SEQ ID NOs: 2, 4, 6, 8, 10, 12 or 14 or an amino acid sequence having at least 90%, 91%, 92%, 93%, 94%, 95%, 96%, 97%, 98%, or 99% sequence identity to the amino acid sequence selected from any one of SEQ ID NOs: 2, 4, 6, 8, 10, 12 or 14. In some of any of the provided embodiments, the antigen-binding domain contains the amino acid sequence selected from any one of SEQ ID NOs: 2, 4, 6, 8, 10, 12 or 14. In some of any of the provided embodiments, the antigen-binding domain contains the amino acid sequence selected from any one of SEQ ID NOs: 2, 4, 8 or 12 or an amino acid sequence having at least 90%, 91%, 92%, 93%, 94%, 95%, 96%, 97%, 98%, or 99% sequence identity to the amino acid sequence selected from any one of SEQ ID NOs: 2, 4, 8 or 12. In some of any of the provided embodiments, the antigen-binding domain contains the amino acid sequence selected from any one of SEQ ID NOs: 2, 4, 8 or 12. In some of any of the provided embodiments, the antigen-binding domain contains the amino acid sequence set forth in SEQ ID NO: 8 or an amino acid sequence having at least 90%, 91%, 92%, 93%, 94%, 95%, 96%, 97%, 98%, or 99% sequence identity to the amino acid sequence set forth in SEQ ID NO: 8. In some of any of the provided embodiments, the antigen-binding domain contains the amino acid sequence set forth in SEQ ID NO: 8. In some of any of the provided embodiments, the antigen-binding domain is encoded by the nucleotide sequence set forth in SEQ ID NO:264.

In some of any of the provided embodiments, the intracellular signaling region contains an intracellular cytoplasmic signaling domain. In some of any of the provided embodiments, the intracellular signaling domain is capable of inducing a primary activation signal in a T cell, is a T cell receptor (TCR) component and/or contains an immunoreceptor tyrosine-based activation motif (ITAM). In some of any of the provided embodiments, the intracellular signaling domain is or contains a cytoplasmic signaling domain of a zeta chain of a CD3-zeta (CD3ζ) chain or a functional variant or signaling portion thereof.

In some of any of the provided embodiments, the intracellular signaling domain is human or is derived from a human protein. In some of any of the provided embodiments, the intracellular signaling domain is or contains the sequence set forth in SEQ ID NO:20 or a sequence of amino acids that has at least 90%, 91%, 92%, 93%, 94%, 95%, 96%, 97%, 98%, or 99% sequence identity to SEQ ID NO:20.

In some of any of the provided embodiments, the intracellular signaling region further contains a costimulatory signaling region. In some of any of the provided embodiments, the costimulatory signaling region contains an intracellular signaling domain of a T cell costimulatory molecule or a signaling portion thereof. In some of any of the provided embodiments, the costimulatory signaling region contains an intracellular signaling domain of a CD28, a 4-1BB or an ICOS or a signaling portion thereof. In some of any of the provided embodiments, the costimulatory signaling region contains an intracellular signaling domain of a 4-1BB or a signaling portion thereof. In some of any of the provided embodiments, the costimulatory signaling region is human or is derived from a human protein. In some of any of the provided embodiments, the costimulatory signaling region contains an intracellular signaling domain of CD28, such as an intracellular signaling domain of human CD28.

In some of any of the provided embodiments, the costimulatory signaling region is or contains the sequence set forth in SEQ ID NO:46 or a sequence of amino acids that exhibits at least 90%, 91%, 92%, 93%, 94%, 95%, 96%, 97%, 98%, or 99% sequence identity to the sequence set forth in SEQ ID NO: 46. In some of any of the provided embodiments, the costimulatory signaling region contains an intracellular signaling domain of 4-1BB. In some of any of the provided embodiments, the costimulatory signaling region is or contains the sequence set forth in SEQ ID NO:19 or a sequence of amino acids that exhibits at least 90%, 91%, 92%, 93%, 94%, 95%, 96%, 97%, 98%, or 99% sequence identity to the sequence set forth in SEQ ID NO: 19.

In some of any of the provided embodiments, the costimulatory signaling region is between the transmembrane domain and the intracellular signaling region. In some of any of the provided embodiments, the transmembrane domain is or contains a transmembrane domain derived from CD4, CD28, or CD8. In some of any of the provided embodiments, the transmembrane domain is or contains a transmembrane domain derived from a CD28. In some of any of the provided embodiments, the transmembrane domain is human or is derived from a human protein. In some of any of the provided embodiments, the transmembrane domain is or contains the sequence set forth in SEQ ID NO:18 or a sequence of amino acids that exhibits at least 90%, 91%, 92%, 93%, 94%, 95%, 96%, 97%, 98%, or 99% sequence identity to SEQ ID NO:18.

Also provided are chimeric antigen receptors containing: (1) an extracellular antigen-binding domain that specifically binds human G-protein coupled receptor (GPRC5D), wherein the extracellular antigen-binding domain contains: (i) a variable heavy chain (V_{H}) containing an amino acid sequence having at least 90%, 91%, 92%, 93%, 94%, 95%, 96%, 97%, 98%, or 99% sequence identity to the V_{H} region amino acid sequence set forth in SEQ ID NO: 27; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO: 28; (2) a spacer set forth in SEQ ID NO:17; (3) a transmembrane domain derived from a human CD28; and (4) an intracellular signaling region containing a cytoplasmic signaling domain of a zeta chain of a CD3-zeta (CD3ζ) chain and an intracellular signaling domain of a T cell costimulatory molecule.

In some of any of the provided embodiments, the V_{H} region contains a CDR-H1, CDR-H2 and CDR-H3 contained within the V_{H} region amino acid sequence set forth in SEQ ID NO: 27; and the V_{L} region contains a CDR-L1, CDR-L2 and CDR-L3 contained within the V_{L} region amino acid sequence set forth in SEQ ID NO: 28; or the V_{H} region contains a CDR-H1, CDR-H2, and CDR-H3 containing the amino acid sequence of SEQ ID NOS:125, 126 and 122, respectively, and the V_{L} region contains a CDR-L1, CDR-L2, and CDR-L3 containing the amino acid sequence of SEQ ID NOS:130, 131 and 132, respectively; the V_{H} region contains a CDR-H1, CDR-H2, and CDR-H3 containing the amino acid sequence of SEQ ID NOS:127, 128 and 129, respectively, and the V_{L} region contains a CDR-L1, CDR-L2, and CDR-L3 containing the amino acid sequence of SEQ ID NOS:133, 134 and 132, respectively; the V_{H} region contains a CDR-H1, CDR-H2, and CDR-H3 containing the amino acid sequence of SEQ ID NOS:120, 121 and 122, respectively, and the V_{L} region contains a CDR-L1, CDR-L2, and CDR-L3 containing the amino acid sequence of SEQ ID NOS:130, 131 and 132, respectively; or the V_{H} region contains a CDR-H1, CDR-H2, and CDR-H3 containing the amino acid sequence of SEQ ID NOS:123, 124 and 122, respectively, and the V_{L} region contains a CDR-L1, CDR-L2, and CDR-L3 containing the amino acid sequence of SEQ ID NOS:130, 131 and 132, respectively. In some of any of the provided embodiments, the V_{H} region contains a CDR-H1, CDR-H2 and CDR-H3 contained within the V_{H} region amino acid sequence set forth in SEQ ID NO: 27; and the V_{L} region contains a CDR-L1, CDR-L2 and CDR-L3 contained within the V_{L} region amino acid sequence set forth in SEQ ID NO: 28; or the V_{H} region contains a CDR-H1, CDR-H2, and CDR-H3 containing the amino acid sequence of SEQ ID NOS:125, 126 and 122, respectively, and the V_{L} region contains a CDR-L1, CDR-L2, and CDR-L3 containing the amino acid sequence of SEQ ID NOS:130, 131 and 132, respectively.

Also provided are chimeric antigen receptors containing: (1) an extracellular antigen-binding domain that specifically binds human G-protein coupled receptor (GPRC5D), wherein the extracellular antigen-binding domain contains: a variable heavy (V_{H}) region containing a CDR-H1, CDR-H2 and CDR-H3 contained within the V_{H} region amino acid sequence set forth in SEQ ID NO: 27; and a variable light (V_{L}) region containing a CDR-L1, CDR-L2 and CDR-L3 contained within the V_{L} region amino acid sequence set forth in SEQ ID NO: 28; or a V_{H} region containing a CDR-H1, CDR-H2, and CDR-H3 containing the amino acid sequence of SEQ ID NOS:125, 126 and 122, respectively, and a V_{L} region containing a CDR-L1, CDR-L2, and CDR-L3 containing the amino acid sequence of SEQ ID NOS:130, 131 and 132, respectively; a V_{H} region containing a CDR-H1, CDR-H2, and CDR-H3 containing the amino acid sequence of SEQ ID NOS:127, 128 and 129, respectively, and a V_{L} region containing a CDR-L1, CDR-L2, and CDR-L3 containing the amino acid sequence of SEQ ID NOS:133, 134 and 132, respectively; a V_{H} region containing a CDR-H1, CDR-H2, and CDR-H3 containing the amino acid sequence of SEQ ID NOS:120, 121 and 122, respectively, and a V_{L} region containing a CDR-L1, CDR-L2, and CDR-L3 containing the amino acid sequence of SEQ ID NOS:130, 131 and 132, respectively; or a V_{H} region containing a CDR-H1, CDR-H2, and CDR-H3 containing the amino acid sequence of SEQ ID NOS:123, 124 and 122, respectively, and a V_{L} region containing a CDR-L1, CDR-L2, and CDR-L3 containing the amino acid sequence of SEQ ID NOS:130, 131 and 132, respectively; (2) a spacer set forth in SEQ ID NO:17; (3) a transmembrane domain derived from a human CD28; and (4) an intracellular signaling region containing a cytoplasmic signaling domain of a zeta chain of a human CD3-zeta (CD3ζ) chain and an intracellular signaling domain of a human CD28 or a human 4-1BB.

Also provided are chimeric antigen receptors containing: (1) an extracellular antigen-binding domain that specifically binds human G-protein coupled receptor (GPRC5D), wherein the extracellular antigen-binding domain contains: a variable heavy (V_{H}) region containing a CDR-H1, CDR-H2 and CDR-H3 contained within the V_{H} region amino acid sequence set forth in SEQ ID NO: 27; and a variable light (V_{L}) region containing a CDR-L1, CDR-L2 and CDR-L3 contained within the V_{L} region amino acid sequence set forth in SEQ ID NO: 28; or a V_{H} region containing a CDR-H1, CDR-H2, and CDR-H3 containing the amino acid sequence of SEQ ID NOS:125, 126 and 122, respectively, and a V_{L} region containing a CDR-L1, CDR-L2, and CDR-L3 containing the amino acid sequence of SEQ ID NOS:130, 131 and 132, respectively; (2) a spacer set forth in SEQ ID NO:17; (3) a transmembrane domain derived from a human CD28; and (4) an intracellular signaling region containing a cytoplasmic signaling domain of a zeta chain of a human CD3-zeta (CD3ζ) chain and an intracellular signaling domain of a human CD28. Also provided are chimeric antigen receptors containing: (1) an extracellular antigen-binding domain that specifically binds human G-protein coupled receptor (GPRC5D), wherein the extracellular antigen-binding domain contains: a variable heavy (V_{H}) region containing a CDR-H1, CDR-H2 and CDR-H3 contained within the V_{H} region amino acid sequence set forth in SEQ ID NO: 27; and a variable light (V_{L}) region containing a CDR-L1, CDR-L2 and CDR-L3 contained within the V_{L} region amino acid sequence set forth in SEQ ID NO: 28; or a V_{H} region containing a CDR-H1, CDR-H2, and CDR-H3 containing the amino acid sequence of SEQ ID NOS:125, 126 and 122, respectively, and a V_{L} region containing a CDR-L1, CDR-L2, and CDR-L3 containing the amino acid sequence of SEQ ID NOS:130, 131 and 132, respectively; (2) a spacer set forth in SEQ ID NO:17; (3) a transmembrane domain derived from a human CD28; and (4) an intracellular signaling region containing a cytoplasmic signaling domain of a zeta chain of a human CD3-zeta (CD3ζ) chain and an intracellular signaling domain of a human 4-1BB.

In some of any of the provided embodiments, the extracellular antigen-binding domain contains the V_{H} region amino acid sequence set forth in SEQ ID NO:27 and the V_{L} region amino acid sequence set forth in SEQ ID NO:28; and/or in some of any of the provided embodiments, the extracellular antigen-binding domain contains an scFv set forth in SEQ ID NO:7 or SEQ ID NO:8. In some of any of the provided embodiments, the extracellular antigen-binding domain contains the V_{H} region amino acid sequence set forth in SEQ ID NO:27 and the V_{L} region amino acid sequence set forth in SEQ ID NO:28; and the extracellular antigen-binding domain contains an scFv set forth in SEQ ID NO:7. In some of any of the provided embodiments, the extracellular antigen-binding domain contains the V_{H} region amino acid sequence set forth in SEQ ID NO:27 and the V_{L} region amino acid sequence set forth in SEQ ID NO:28; and the extracellular antigen-binding domain contains an scFv set forth in SEQ ID NO:8.

In some of any of the provided embodiments, the transmembrane domain is or contains the sequence set forth in SEQ ID NO:18 or a sequence of amino acids that exhibits at least 90%, 91%, 92%, 93%, 94%, 95%, 96%, 97%, 98%, or 99% sequence identity to SEQ ID NO:18. In some of any of the provided embodiments, the transmembrane domain is or contains the sequence set forth in SEQ ID NO:18.

In some of any of the provided embodiments, the intracellular signaling region contains the sequence set forth in SEQ ID NO:20 or a sequence of amino acids that has at least 90%, 91%, 92%, 93%, 94%, 95%, 96%, 97%, 98%, or 99% sequence identity to SEQ ID NO:20 and the sequence set forth in SEQ ID NO:46 or a sequence of amino acids that exhibits at least 90%, 91%, 92%, 93%, 94%, 95%, 96%, 97%, 98%, or 99% sequence identity to the sequence set forth in SEQ ID NO: 46. In some of any of the provided embodiments, the intracellular signaling region is or contains the sequences set forth in SEQ ID NO:20 and SEQ ID NO:46. In some of any of the provided embodiments, the intracellular signaling region contains the sequence set forth in SEQ ID NO:20 or a sequence of amino acids that has at least 90%, 91%, 92%, 93%, 94%, 95%, 96%, 97%, 98%, or 99% sequence identity to SEQ ID NO:20 and the sequence set forth in SEQ ID NO:19 or a sequence of amino acids that exhibits at least 90%, 91%, 92%, 93%, 94%, 95%, 96%, 97%, 98%, or 99% sequence identity to the sequence set forth in SEQ ID NO: 19. In some of any of the provided embodiments, the intracellular signaling region is or contains the sequences set forth in SEQ ID NO:20 and SEQ ID NO:19.

In some of any of the provided embodiments, the chimeric antigen receptor contains from its N to C terminus in order: the antigen-binding domain, the spacer, the transmembrane domain and the intracellular signaling region.

Also provided are polynucleotides containing a sequence of nucleotides encoding any of the chimeric antigen receptors provided herein.

In some of any of the provided embodiments, the nucleic acid encoding the spacer contains at least one modified splice donor and/or splice acceptor site, said modified splice donor and/or acceptor site containing one or more nucleotide modifications corresponding to a reference splice donor site and/or reference splice acceptor site contained in the sequence set forth in SEQ ID NO:73. In some of any of the provided embodiments, the one or more nucleotide modifications contain an amino acid substitution. In some of any of the provided embodiments, the reference splice donor and/or reference splice acceptor sites are canonical, non-canonical, or cryptic splice sites. In some of any of the provided embodiments, the reference splice donor and/or reference splice acceptor site(s) has a splice site prediction score of at least or about 0.4, 0.5, 0.6, 0.70, 0.75, 0.80, 0.85, 0.90, 0.95, 0.99, or 1.0; and/or the reference splice donor and/or reference splice acceptor site(s) is/are predicted to be involved in a splice event with a probability of at least 40%, 50%, 60%, 70%, 75%, 80%, 85%, 90%, 95%, 99%, or 100%.

In some of any of the provided embodiments, the reference splice donor site contains the sequence aatctaagtacggac (SEQ ID NO: 176), tcaactggtacgtgg (SEQ ID NO:177), acaattagtaaggca (SEQ ID NO:178) and/or accacaggtgtatac (SEQ ID NO:179); and/or the reference splice acceptor site contains the sequence aagtttctttctgtattccaggctgaccgtggataaatctc (SEQ ID NO:180) and/or gggcaacgtgttctcttgcagtgtcatgcacgaagccctgc (SEQ ID NO:181).

In some of any of the provided embodiments, the reference splice donor and/or reference splice acceptor site(s) has a splice site prediction score of at least or about 0.70, 0.75, 0.80, 0.85, 0.90, 0.95, 0.99, or 1.0; and/or In some of any of the provided embodiments, the reference splice donor and/or reference splice acceptor site(s) is/are predicted to be involved in a splice event with a probability of at least 70%, 75%, 80%, 85%, 90%, 95%, 99%, or 100%.

In some of any of the provided embodiments, the reference splice donor site contains the sequence tcaactggtacgtgg (SEQ ID NO:177); and/or the reference splice acceptor site contains the sequence aagtttctttctgtattccaggctgaccgtggataaatctc (SEQ ID NO:180).

In some of any of the provided embodiments, at least one of the one or more nucleotide modifications are within 1, 2, 3, 4, 5, 6, 7, 8, 9 or 10 residues of the splice site junction of the reference splice acceptor and/or reference splice donor site.

In some of any of the provided embodiments, the one or more nucleotide modifications is silent and/or results in a degenerate codon compared to SEQ ID NO:73 and/or does not change the amino acid sequence of the encoded spacer.

In some of any of the provided embodiments, the modified splice donor site is set forth in agtctaaatacggac (SEQ ID NO:182), tcaactggtatgtgg (SEQ ID NO:183), accatctccaaggcc (SEQ ID NO:184) and/or gccccaggtttacac (SEQ ID NO:185); and/or the modified splice acceptor site is set forth in cagtttcttcctgtatagtagactcaccgtggataaatcaa (SEQ ID NO:186), gggcaacgtgttcagctgcagcgtgatgcacgaggccctgc (SEQ ID NO: 187) and/or cgccttgtcctccttgtcccgctcctcctgttgccggacct (SEQ ID NO:188). In some of any of the provided embodiments, the modified splice donor site is set forth in tcaactggtatgtgg (SEQ ID NO:183) and/or the modified acceptor site is set forth in cagtttcttcctgtatagtagactcaccgtggataaatcaa (SEQ ID NO:186) and/or cgccttgtcctccttgtcccgctcctcctgttgccggacct (SEQ ID NO:188).

In some of any of the provided embodiments, the spacer is encoded by a sequence of nucleotide set forth in SEQ ID NO:74 (also set forth in SEQ ID NO:48) or a portion thereof. In some of any of the provided embodiments, the spacer is encoded by a sequence of nucleotide set forth in SEQ ID NO:73 or a portion thereof. In some of any of the provided embodiments, the spacer is encoded by a sequence of nucleotide set forth in SEQ ID NO:74 or a portion thereof. In some of any of the provided embodiments, the spacer is encoded by a sequence of nucleotide set forth in SEQ ID NO:283 or a portion thereof. In some of any of the provided embodiments, the spacer is encoded by a sequence of nucleotide set forth in SEQ ID NO:284 or a portion thereof. In some of any of the provided embodiments, the spacer is encoded by a sequence of nucleotide set forth in SEQ ID NO:305 or a portion thereof.

In some of any of the provided embodiments, upon expression of the polynucleotide in a cell, the transcribed RNA, optionally messenger RNA (mRNA), from the polynucleotide, exhibits at least 70%, 75%, 80%, 85%, 90%, or 95% RNA homogeneity.

In some of any of the provided embodiments, upon expression in a cell, the transcribed RNA, optionally messenger RNA (mRNA), from the polynucleotide exhibits reduced heterogeneity compared to the heterogeneity of the mRNA transcribed from a reference polynucleotide, said reference polynucleotide encoding the same amino acid sequence as the polynucleotide, wherein the reference polynucleotide differs by the presence of one or more splice donor site and/or one or more splice acceptor site in the nucleic acid encoding the spacer and/or contains one or more nucleotide modifications compared to the polynucleotide and/or contains the spacer set forth in SEQ ID NO:73. In some of any of the provided embodiments, the RNA heterogeneity is reduced by greater than or greater than about 10%, 15%, 20%, 25%, 30%, 40%, 50% or more. In some of any of the provided embodiments, the transcribed RNA, optionally messenger RNA (mRNA), from the reference polynucleotide exhibits greater than or greater than about 10%, 15%, 20%, 25%, 30%, 40%, 50% or more RNA heterogeneity. In some of any of the provided embodiments, the RNA homogeneity and/or heterogeneity is determined by agarose gel electrophoresis, chip-based capillary electrophoresis, analytical ultracentrifugation, field flow fractionation, or liquid chromatography.

In some of any of the provided embodiments, the polynucleotide is codon-optimized for expression in a human cell.

In some of any of the provided embodiments, the chimeric receptor is a first chimeric receptor and the polynucleotide further contains a sequence of nucleotides encoding a second chimeric antigen receptor. Thus, also provided herein are polynucleotides that encode a first chimeric receptor that is directed against GPRC5D, including any as provided herein, and a second chimeric receptor. In some of any of the provided embodiments, the first and second chimeric receptors are separated by one or more multicistronic element(s). In some of any of the provided embodiments, the one or more multicistronic element is or contains a ribosome skip sequence. In some embodiments, the ribosome skip sequence is a T2A, a P2A, an E2A, or an F2A element. In some of any of the provided embodiments, the one or more multicistronic element contains the amino acid sequence set forth in SEQ ID NO:37. In some of any of the provided embodiments, the one or more multicistronic element is encoded by a nucleotide sequence selected from among SEQ ID NOS: 44, 45, and 319. In some of any of the provided embodiments, the nucleotide sequence encoding the one or more multicistronic element is codon diverged. In some of any of the provided embodiments, nucleotide sequence encoding the multicistronic element is or comprises the sequence set forth in SEQ ID NO:319.

In some of any of the provided embodiments, the second chimeric receptor contains an extracellular antigen binding domain that specifically binds a second antigen, such as other than GPRC5D, expressed on or associated with multiple myeloma. In some of any of the provided embodiments, the second CAR contains an extracellular antigen binding domain that binds the second antigen, a spacer, a transmembrane domain, and an intracellular signaling region. In some of any of the provided embodiments, the second antigen is selected from B cell maturation antigen (BCMA), CD38, CD138, CS-1, BAFF-R, TACI or FcRH5. In some of any of the provided embodiments, the second antigen is BCMA.

In some of any of the provided embodiments, the second CAR contains: (1) an extracellular antigen-binding domain that specifically binds BCMA, wherein the extracellular antigen-binding domain contains: (i) a variable heavy chain (V_{H}) containing an amino acid sequence having at least 90%, 91%, 92%, 93%, 94%, 95%, 96%, 97%, 98%, or 99% sequence identity to the V_{H} region amino acid sequence set forth in any of SEQ ID NOs: 189, 191, 193, 195 or 197; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO: 190, 192, 194, 196 or 198; (2) a spacer; (3) a transmembrane; and (4) an intracellular signaling region. In some of any of the provided embodiments, the V_{H} region of the second CAR contains a CDR-H1, CDR-H2 and CDR-H3 contained within the V_{H} region amino acid sequence set forth in any of SEQ ID NOs: 189, 191, 193, 195 or 197; and the V_{L} region contains a CDR-L1, CDR-L2 and CDR-L3 contained within the V_{L} region amino acid sequence set forth in any of SEQ ID NOs: 190, 192, 194, 196 or 198.

In some of any of the provided embodiments, the second CAR contains: (1) an extracellular antigen-binding domain that specifically binds BCMA, wherein the extracellular antigen-binding domain contains: (i) a variable heavy chain (V_{H}) containing an amino acid sequence having at least 90%, 91%, 92%, 93%, 94%, 95%, 96%, 97%, 98%, or 99% sequence identity to the V_{H} region amino acid sequence set forth in SEQ ID NO: 197; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO:198; (2) a spacer; (3) a transmembrane domain; and (4) an intracellular signaling region. In some of any of the provided embodiments, the second CAR contains: (1) an extracellular antigen-binding domain that specifically binds BCMA, wherein the extracellular antigen-binding domain contains: (i) a variable heavy chain (V_{H}) containing an amino acid sequence having at least 90%, 91%, 92%, 93%, 94%, 95%, 96%, 97%, 98%, or 99% sequence identity to the V_{H} region amino acid sequence set forth in SEQ ID NO: 197; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO:198; (2) a spacer set forth in SEQ ID NO: 17; (3) a transmembrane domain derived from a human CD28; and (4) an intracellular signaling region containing a cytoplasmic signaling domain of a zeta chain of a human CD3-zeta (CD3ζ) chain and an intracellular signaling domain of a human 4-1BB. In some of any of the provided embodiments, the V_{H} region of the second CAR contains a CDR-H1, CDR-H2 and CDR-H3 contained within the V_{H} region amino acid sequence set forth in any of SEQ ID NOs: 197; and the V_{L} region contains a CDR-L1, CDR-L2 and CDR-L3 contained within the V_{L} region amino acid sequence set forth in any of SEQ ID NOs:198. In some of any of the provided embodiments, the second CAR is or comprises the amino acid sequence set forth in SEQ ID NO:251. In some of any of the provided embodiments, the second CAR is encoded by the nucleotide sequence set forth in SEQ ID NO:246.

In some of any of the provided embodiments, the second CAR contains: (1) an extracellular antigen-binding domain that specifically binds BCMA, wherein the extracellular antigen-binding domain contains: (i) a variable heavy chain (V_{H}) containing a heavy chain complementarity determining region 1 (CDR-H1) containing the amino acid sequence selected from any one of SEQ ID NOs: 199, 202, 206, 209, 212 or 215; (b) a heavy chain complementarity determining region 2 (CDR-H2) containing the amino acid sequence selected from any one of SEQ ID NOs: 200, 203, 207, 210, 213 or 216; and (c) a heavy chain complementarity determining region 3 (CDR-H3) containing the amino acid sequence selected from any one of SEQ ID NOs: 201, 204, 205, 208, 211, 214 or 217; and (ii) a variable light chain (V_{L}) region containing a light chain complementarity determining region 1 (CDR-L1) containing the amino acid sequence selected from any one of SEQ ID NOs: 218, 221, 224, 227, 230, 233 or 235; (b) a light chain complementarity determining region 2 (CDR-L2) containing the amino acid sequence selected from any one of SEQ ID NOs: 219, 222, 225, 228, 231, 234 or 236; and (c) a light chain complementarity determining region 3 (CDR-L3) containing the amino acid sequence selected from any one of SEQ ID NOs: 220, 223, 226, 229 or 232; (2) a spacer; (3) a transmembrane domain; and (4) an intracellular signaling region.

In some of any of the provided embodiments, the V_{H} region of the second CAR contains a CDR-H1, CDR-H2, and CDR-H3 containing the amino acid sequence of SEQ ID NOS:199, 200 and 201, respectively, and the V_{L} region of the second CAR contains a CDR-L1, CDR-L2, and CDR-L3 containing the amino acid sequence of SEQ ID NOS:218, 219 and 220, respectively; the V_{H} region of the second CAR contains a CDR-H1, CDR-H2, and CDR-H3 containing the amino acid sequence of SEQ ID NOS:202, 203, 204, respectively, and the V_{L} region of the second CAR contains a CDR-L1, CDR-L2, and CDR-L3 containing the amino acid sequence of SEQ ID NOS:221, 222 and 223, respectively; the V_{H} region of the second CAR contains a CDR-H1, CDR-H2, and CDR-H3 containing the amino acid sequence of SEQ ID NOS:199, 200, 205, respectively, and the V_{L} region of the second CAR contains a CDR-L1, CDR-L2, and CDR-L3 containing the amino acid sequence of SEQ ID NOS:224, 225, and 226, respectively; the V_{H} region of the second CAR contains a CDR-H1, CDR-H2, and CDR-H3 containing the amino acid sequence of SEQ ID NOS:206, 207, 208, respectively, and the V_{L} region of the second CAR contains a CDR-L1, CDR-L2, and CDR-L3 containing the amino acid sequence of SEQ ID NOS:227, 228 and 229, respectively; the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; the V_{H} region of the second CAR contains a CDR-H1, CDR-H2, and CDR-H3 containing the amino acid sequence of SEQ ID NOS:212, 213 and 214, respectively, and the V_{L} region of the second CAR contains a CDR-L1, CDR-L2, and CDR-L3 containing the amino acid sequence of SEQ ID NOS:233, 234 and 229, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively.

In some of any of the provided embodiments, the V_{H} region of the encoded second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively. In some of any of the provided embodiments, the V_{H} region of the encoded second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively. In some of any of the provided embodiments, the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively.

In some of any of the provided embodiments, the V_{H} region and V_{L} region of the encoded second CAR contains the amino acid sequences set forth in SEQ ID NO:189 and SEQ ID NO:190, respectively, or a sequence of amino acids that exhibits at least 90%, 91%, 92%, 93%, 94%, 95%, 96%, 97%, 98%, or 99% sequence identity to SEQ ID NO:189 and SEQ ID NO:190; the V_{H} region and V_{L} region of the second CAR contains the amino acid sequences set forth in SEQ ID NO:191 and SEQ ID NO:192, respectively, or a sequence of amino acids that exhibits at least 90%, 91%, 92%, 93%, 94%, 95%, 96%, 97%, 98%, or 99% sequence identity to SEQ ID NO:191 and SEQ ID NO:192; the V_{H} region and V_{L} region of the second CAR contains the amino acid sequences set forth in SEQ ID NO:193 and SEQ ID NO:194, respectively, or a sequence of amino acids that exhibits at least 90%, 91%, 92%, 93%, 94%, 95%, 96%, 97%, 98%, or 99% sequence identity to SEQ ID NO:193 and SEQ ID NO:194; the V_{H} region and V_{L} region of the second CAR contains the amino acid sequences set forth in SEQ ID NO:195 and SEQ ID NO:196, respectively, or a sequence of amino acids that exhibits at least 90%, 91%, 92%, 93%, 94%, 95%, 96%, 97%, 98%, or 99% sequence identity to SEQ ID NO:195 and SEQ ID NO:196; or the V_{H} region and V_{L} region of the second CAR contains the amino acid sequences set forth in SEQ ID NO:197 and SEQ ID NO:198, respectively, or a sequence of amino acids that exhibits at least 90%, 91%, 92%, 93%, 94%, 95%, 96%, 97%, 98%, or 99% sequence identity to SEQ ID NO:197 and SEQ ID NO:198.

In some of any of the provided embodiments, the V_{H} region and V_{L} region of the encoded second CAR contains the amino acid sequences set forth in SEQ ID NO:189 and SEQ ID NO:190, respectively; the V_{H} region and V_{L} region of the second CAR contains the amino acid sequences set forth in SEQ ID NO:191 and SEQ ID NO:192; the V_{H} region and V_{L} region of the second CAR contains the amino acid sequences set forth in SEQ ID NO:193 and SEQ ID NO:194; the V_{H} region and V_{L} region of the second CAR contains the amino acid sequences set forth in SEQ ID NO:195 and SEQ ID NO:196; or the V_{H} region and V_{L} region of the second CAR contains the amino acid sequences set forth in SEQ ID NO:197 and SEQ ID NO:198, respectively. In some of any of the provided embodiments, the V_{H} region and V_{L} region of the encoded second CAR contain the amino acid sequences set forth in SEQ ID NO:197 and SEQ ID NO:198, respectively.

In some of any of the provided embodiments, the extracellular antigen-binding domain of the encoded second CAR is a single chain antibody fragment. In some of any of the provided embodiments, the encoded fragment is or contains a single chain variable fragment (scFv). In some of any of the provided embodiments, the V_{H} region and the V_{L} region of the encoded second CAR are joined by a flexible linker. In some of any of the provided embodiments, the scFv of the encoded second CAR contains a linker containing the amino acid sequence GGGGSGGGGSGGGGS (SEQ ID NO:52). In some of any of the provided embodiments, the scFv of the encoded second CAR contains a linker containing the amino acid sequence GGGGSGGGGSGGGGSGGGGS (SEQ ID NO:320). In some of any of the provided embodiments, the V_{H} region is amino-terminal to the V_{L} region in the encoded second CAR. In some of any of the provided embodiments, the V_{H} region is carboxy-terminal to the V_{L} region in the encoded second CAR.

In some of any of the provided embodiments, the antigen-binding domain of the encoded second CAR contains the amino acid sequence selected from any one of SEQ ID NOs: 227, 238, 239, 240 or 241 or an amino acid sequence having at least 90%, 91%, 92%, 93%, 94%, 95%, 96%, 97%, 98%, or 99% sequence identity to the amino acid sequence selected from any one of SEQ ID NOs: 227, 238, 239, 240 or 241. In some of any of the provided embodiments, the antigen-binding domain of the encoded second CAR contains the amino acid sequence selected from any one of SEQ ID NOs: 227, 238, 239, 240 or 241. In some of any of the provided embodiments, the antigen-binding domain of the encoded second CAR contains the amino acid sequence set forth in SEQ ID NO: 241 or an amino acid sequence having at least 90%, 91%, 92%, 93%, 94%, 95%, 96%, 97%, 98%, or 99% sequence identity to the amino acid sequence set forth in SEQ ID NO: 241. In some of any of the provided embodiments, the antigen-binding domain of the encoded second CAR contains the amino acid sequence set forth in SEQ ID NO: 241.

In some of any of the provided embodiments, the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively; and/or the V_{H} region and V_{L} region of the second CAR contains the amino acid sequences set forth in SEQ ID NO:197 and SEQ ID NO:198, respectively; and/or the antigen-binding domain of the second CAR contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241. In some of any of the provided embodiments, the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; and/or the V_{H} region and V_{L} region of the second CAR contains the amino acid sequences set forth in SEQ ID NO:197 and SEQ ID NO:198, respectively; and/or the antigen-binding domain of the second CAR contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241. In some of any of the provided embodiments, the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively; and/or the V_{H} region and V_{L} region of the second CAR contains the amino acid sequences set forth in SEQ ID NO:197 and SEQ ID NO:198, respectively; and/or the antigen-binding domain of the second CAR contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241.

In some of any of the provided embodiments, the transmembrane domain of the encoded second CAR is or contains a transmembrane domain derived from CD4, CD28, or CD8, optionally from a human CD4, a human CD28 or a human CD8.

In some of any of the provided embodiments, the transmembrane domain of the encoded second CAR is or contains a transmembrane domain derived from a human CD28 and/or is or contains the sequence set forth in SEQ ID NO:18 or a sequence of amino acids that exhibits at least 90%, 91%, 92%, 93%, 94%, 95%, 96%, 97%, 98%, or 99% sequence identity to SEQ ID NO:18. In some of any of the provided embodiments, the transmembrane domain of the encoded second CAR is or contains the sequence set forth in SEQ ID NO:18.

In some of any of the provided embodiments, the intracellular signaling region of the encoded second CAR contains an intracellular signaling domain. In some of any of the provided embodiments, the intracellular signaling domain of the encoded second CAR is capable of inducing a primary activation signal in a T cell, is a T cell receptor (TCR) component and/or contains an immunoreceptor tyrosine-based activation motif (ITAM). In some of any of the provided embodiments, the intracellular signaling domain of the encoded second CAR is or contains a cytoplasmic signaling domain of a zeta chain of a CD3-zeta (CD3ζ) chain or a functional variant or signaling portion thereof, optionally a human CD3 zeta chain. In some of any of the provided embodiments, the intracellular signaling region of the encoded second CAR contains the sequence set forth in SEQ ID NO:20 or a sequence of amino acids that has at least 90%, 91%, 92%, 93%, 94%, 95%, 96%, 97%, 98%, or 99% sequence identity to SEQ ID NO:20.

In some of any of the provided embodiments, the intracellular signaling region of the encoded second CAR further contains a costimulatory signaling region. In some of any of the provided embodiments, the costimulatory signaling region of the encoded second CAR contains an intracellular signaling domain of a T cell costimulatory molecule or a signaling portion thereof. In some of any of the provided embodiments, the costimulatory signaling region of the encoded second CAR contains an intracellular signaling domain of a CD28, a 4-1BB or an ICOS or a signaling portion thereof, optionally a human CD28, a human 4-1BB, or a human ICOS. In some of any of the provided embodiments, the costimulatory signaling region of the encoded second CAR contains an intracellular signaling domain of a 4-1BB or a signaling portion thereof. In some of any of the provided embodiments, at least one of the first chimeric antigen receptor and the second chimeric antigen receptor contains an intracellular signaling region containing an intracellular signaling domain of 4-1BB or a signaling portion thereof, optionally of human 4-1BB.

In some of any of the provided embodiments, the costimulatory signaling region of the encoded second CAR contains: an intracellular signaling domain of a human CD28; and/or the sequence set forth in SEQ ID NO:46 or a sequence of amino acids that exhibits at least 90%, 91%, 92%, 93%, 94%, 95%, 96%, 97%, 98%, or 99% sequence identity to the sequence set forth in SEQ ID NO: 46.

In some of any of the provided embodiments, the costimulatory signaling region of the encoded second CAR contains: an intracellular signaling domain of a human 4-1BB; and/or the sequence set forth in SEQ ID NO:19 or a sequence of amino acids that exhibits at least 90%, 91%, 92%, 93%, 94%, 95%, 96%, 97%, 98%, or 99% sequence identity to the sequence set forth in SEQ ID NO: 19.

In some of any of the provided embodiments, the encoded second chimeric antigen receptor contains from its N to C terminus in order: the antigen-binding domain, the spacer, the transmembrane domain and the intracellular signaling region.

In some of any of the provided embodiments, at least one of the polynucleotide sequence encoding the first chimeric antigen receptor and the polynucleotide sequence encoding the second chimeric antigen receptor is codon diverged. In some of any of the provided embodiments, the polynucleotide sequence encoding the first chimeric antigen receptor and the polynucleotide sequence encoding the second chimeric antigen receptor have no more than about 30, no more than about 20, or no more than about 10 consecutive base pairs of sequence homology.

In some of any of the provided embodiments, the sequence of nucleotides encoding the CAR is operably linked to a promoter to control expression of the encoded CAR when expressed from a cell introduced with the polynucleotide, optionally wherein the promoter is a heterologous promoter, optionally wherein the heterologous promoter is or contains a human elongation factor 1 alpha (EF1α) promoter or an MND promoter or a variant thereof.

In some of any of the provided embodiments, wherein the polynucleotide encodes two CARs, the sequence of nucleotides encoding the first CAR is operably linked to a first promoter to control expression of the first CAR when expressed from a cell introduced with the polynucleotide and the sequence of nucleotides encoding the second CAR is operably linked to a second promoter to control expression of the second CAR when expressed from a cell introduced with the polynucleotide. In some embodiments, the first and second promoter independently is a heterologous promoter, such as wherein the heterologous promoter is or contains a human elongation factor 1 alpha (EF1α) promoter or an MND promoter or a variant thereof. In some of such embodiments, the first and second promoters are the same. In some of such embodiments, the first and second promoter are different.

Also provided are vectors containing any of the provided polynucleotides. In some of any of the provided embodiments, the vector is a viral vector. In some of any of the provided embodiments, the viral vector is a lentiviral vector or a retroviral vector.

Also provided are engineered cells containing any of the chimeric antigen receptors provided herein. In some of any of the provided embodiments, the engineered cell contains a chimeric antigen receptor provided herein and further contains a polynucleotide containing a sequence of nucleotides encoding a second chimeric antigen receptor.

Also provided are engineered cells containing any of the polynucleotides provided herein.

In some of any of the provided embodiments, the second chimeric receptor of the provided cell contains an extracellular antigen binding domain that specifically binds a second antigen expressed on or associated with multiple myeloma. In some of any of the provided embodiments, the second CAR contains the extracellular antigen binding domain that binds the second antigen, a spacer, a transmembrane domain, and an intracellular signaling region. In some of any of such embodiments, the second antigen is selected from B cell maturation antigen (BCMA), CD38, CD138, CS-1, BAFF-R, TACI or FcRH5. In some of any of the provided embodiments, the second antigen is BCMA.

In some of any of the provided embodiments of engineered cells, the second CAR contains: (1) an extracellular antigen-binding domain that specifically binds BCMA, wherein the extracellular antigen-binding domain contains: (i) a variable heavy chain (V_{H}) containing an amino acid sequence having at least 90%, 91%, 92%, 93%, 94%, 95%, 96%, 97%, 98%, or 99% sequence identity to the V_{H} region amino acid sequence set forth in any of SEQ ID NOs: 189, 191, 193, 195 or 197; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO: 190, 192, 194, 196 or 198; (2) a spacer; (3) a transmembrane; and (4) an intracellular signaling region. In some of such embodiments, the V_{H} region contains a CDR-H1, CDR-H2 and CDR-H3 contained within the V_{H} region amino acid sequence set forth in any of SEQ ID NOs: 189, 191, 193, 195 or 197; and the V_{L} region contains a CDR-L1, CDR-L2 and CDR-L3 contained within the V_{L} region amino acid sequence set forth in any of SEQ ID NOs: 190, 192, 194, 196 or 198.

In some of any of the provided embodiments of engineered cells, the second CAR contains: (1) an extracellular antigen-binding domain that specifically binds BCMA, wherein the extracellular antigen-binding domain contains: (i) a variable heavy chain (V_{H}) containing a heavy chain complementarity determining region 1 (CDR-H1) containing the amino acid sequence selected from any one of SEQ ID NOs: 199, 202, 206, 209, 212 or 215; (b) a heavy chain complementarity determining region 2 (CDR-H2) containing the amino acid sequence selected from any one of SEQ ID NOs: 200, 203, 207, 210, 213 or 216; and (c) a heavy chain complementarity determining region 3 (CDR-H3) containing the amino acid sequence selected from any one of SEQ ID NOs: 201, 204, 205, 208, 211, 214 or 217; and (ii) a variable light chain (V_{L}) region containing a light chain complementarity determining region 1 (CDR-L1) containing the amino acid sequence selected from any one of SEQ ID NOs: 218, 221, 224, 227, 230, 233 or 235; (b) a light chain complementarity determining region 2 (CDR-L2) containing the amino acid sequence selected from any one of SEQ ID NOs: 219, 222, 225, 228, 231, 234 or 236; and (c) a light chain complementarity determining region 3 (CDR-L3) containing the amino acid sequence selected from any one of SEQ ID NOs: 220, 223, 226, 229 or 232; (2) a spacer; (3) a transmembrane domain; and (4) an intracellular signaling region.

In some of any of the provided embodiments of engineered cells, the V_{H} region of the second CAR contains a CDR-H1, CDR-H2, and CDR-H3 containing the amino acid sequence of SEQ ID NOS:199, 200 and 201, respectively, and the V_{L} region of the second CAR contains a CDR-L1, CDR-L2, and CDR-L3 containing the amino acid sequence of SEQ ID NOS:218, 219 and 220, respectively; the V_{H} region of the second CAR contains a CDR-H1, CDR-H2, and CDR-H3 containing the amino acid sequence of SEQ ID NOS:202, 203, 204, respectively, and the V_{L} region of the second CAR contains a CDR-L1, CDR-L2, and CDR-L3 containing the amino acid sequence of SEQ ID NOS:221, 222 and 223, respectively; the V_{H} region of the second CAR contains a CDR-H1, CDR-H2, and CDR-H3 containing the amino acid sequence of SEQ ID NOS:199, 200, 205, respectively, and the V_{L} region of the second CAR contains a CDR-L1, CDR-L2, and CDR-L3 containing the amino acid sequence of SEQ ID NOS:224, 225, and 226, respectively; the V_{H} region of the second CAR contains a CDR-H1, CDR-H2, and CDR-H3 containing the amino acid sequence of SEQ ID NOS:206, 207, 208, respectively, and the V_{L} region of the second CAR contains a CDR-L1, CDR-L2, and CDR-L3 containing the amino acid sequence of SEQ ID NOS:227, 228 and 229, respectively; the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; the V_{H} region of the second CAR contains a CDR-H1, CDR-H2, and CDR-H3 containing the amino acid sequence of SEQ ID NOS:212, 213 and 214, respectively, and the V_{L} region of the second CAR contains a CDR-L1, CDR-L2, and CDR-L3 containing the amino acid sequence of SEQ ID NOS:233, 234 and 229, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively.

In some of any of the provided embodiments of engineered cells, the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively.

In some of any of the provided embodiments of engineered cells, the V_{H} region and V_{L} region of the second CAR contains the amino acid sequences set forth in SEQ ID NO:189 and SEQ ID NO:190, respectively, or a sequence of amino acids that exhibits at least 90%, 91%, 92%, 93%, 94%, 95%, 96%, 97%, 98%, or 99% sequence identity to SEQ ID NO:189 and SEQ ID NO:190; the V_{H} region and V_{L} region of the second CAR contains the amino acid sequences set forth in SEQ ID NO:191 and SEQ ID NO:192, respectively, or a sequence of amino acids that exhibits at least 90%, 91%, 92%, 93%, 94%, 95%, 96%, 97%, 98%, or 99% sequence identity to SEQ ID NO:191 and SEQ ID NO:192; the V_{H} region and V_{L} region of the second CAR contains the amino acid sequences set forth in SEQ ID NO:193 and SEQ ID NO:194, respectively, or a sequence of amino acids that exhibits at least 90%, 91%, 92%, 93%, 94%, 95%, 96%, 97%, 98%, or 99% sequence identity to SEQ ID NO:193 and SEQ ID NO:194; the V_{H} region and V_{L} region of the second CAR contains the amino acid sequences set forth in SEQ ID NO:195 and SEQ ID NO:196, respectively, or a sequence of amino acids that exhibits at least 90%, 91%, 92%, 93%, 94%, 95%, 96%, 97%, 98%, or 99% sequence identity to SEQ ID NO:195 and SEQ ID NO:196; or the V_{H} region and V_{L} region of the second CAR contains the amino acid sequences set forth in SEQ ID NO:197 and SEQ ID NO:198, respectively, or a sequence of amino acids that exhibits at least 90%, 91%, 92%, 93%, 94%, 95%, 96%, 97%, 98%, or 99% sequence identity to SEQ ID NO:197 and SEQ ID NO:198.

In some of any of the provided embodiments of engineered cells, the V_{H} region and V_{L} region of the second CAR contains the amino acid sequences set forth in SEQ ID NO:189 and SEQ ID NO:190, respectively; the V_{H} region and V_{L} region of the second CAR contains the amino acid sequences set forth in SEQ ID NO:191 and SEQ ID NO:192; the V_{H} region and V_{L} region of the second CAR contains the amino acid sequences set forth in SEQ ID NO:193 and SEQ ID NO:194; the V_{H} region and V_{L} region of the second CAR contains the amino acid sequences set forth in SEQ ID NO:195 and SEQ ID NO:196; or the V_{H} region and V_{L} region of the second CAR contains the amino acid sequences set forth in SEQ ID NO:197 and SEQ ID NO:198, respectively.

In some of any of the provided embodiments of engineered cells, the extracellular antigen-binding domain of the second CAR is a single chain antibody fragment. In some of any of such embodiments, the fragment is or contains a single chain variable fragment (scFv).

In some of any of the provided embodiments of engineered cells, the V_{H} region and the V_{L} region of the second CAR are joined by a flexible linker. In some of such embodiments, the linker contains the amino acid sequence GGGGSGGGGSGGGGS (SEQ ID NO:52). In some of such embodiments, the linker contains the amino acid sequence GGGGSGGGGSGGGGSGGGGS (SEQ ID NO:320).

In some of any of the provided embodiments of engineered cells, the V_{H} region is amino-terminal to the V_{L} region in the second CAR. In some of any of the provided embodiments of engineered cells, the V_{H} region is carboxy-terminal to the V_{L} region in the second CAR. In some of any of the provided embodiments of engineered cells, the antigen-binding domain of the second CAR contains the amino acid sequence selected from any one of SEQ ID NOs: 227, 238, 239, 240 or 241 or an amino acid sequence having at least 90%, 91%, 92%, 93%, 94%, 95%, 96%, 97%, 98%, or 99% sequence identity to the amino acid sequence selected from any one of SEQ ID NOs: 227, 238, 239, 240 or 241.

In some of any of the provided embodiments of engineered cells, the antigen-binding domain of the second CAR contains the amino acid sequence selected from any one of SEQ ID NOs: 227, 238, 239, 240 or 241. In some of any of the provided embodiments of engineered cells, the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively; and/or the V_{H} region and V_{L} region of the second CAR contains the amino acid sequences set forth in SEQ ID NO:197 and SEQ ID NO:198, respectively; and/or the antigen-binding domain of the second CAR contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241.

In some of any of the provided embodiments of engineered cells, the antigen-binding domain of the second CAR contains the amino acid set forth in SEQ ID NO: 241. In some of any of the provided embodiments of engineered cells, the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; and/or the V_{H} region and V_{L} region of the second CAR contains the amino acid sequences set forth in SEQ ID NO:197 and SEQ ID NO:198, respectively; and/or the antigen-binding domain of the second CAR contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241.

In some of any of the provided embodiments of engineered cells, the transmembrane domain of the second CAR is or contains a transmembrane domain derived from CD4, CD28, or CD8, optionally from a human CD4, a human CD28 or a human CD8. In some of any of the provided embodiments of engineered cells, the transmembrane domain of the second CAR is or contains a transmembrane domain derived from a human CD28; and/or the transmembrane domain is or contains the sequence set forth in SEQ ID NO:18 or a sequence of amino acids that exhibits at least 90%, 91%, 92%, 93%, 94%, 95%, 96%, 97%, 98%, or 99% sequence identity to SEQ ID NO:18. In some of any of the provided embodiments of engineered cells, the transmembrane domain of the second CAR is or contains the sequence set forth in SEQ ID NO:18.

In some of any of the provided embodiments of engineered cells, the intracellular signaling region of the second CAR contains an intracellular signaling domain.

In some of any of the provided embodiments of engineered cells, the intracellular signaling domain of the second CAR is capable of inducing a primary activation signal in a T cell, is a T cell receptor (TCR) component and/or contains an immunoreceptor tyrosine-based activation motif (ITAM). In some of any of the provided embodiments of engineered cells, the intracellular signaling domain of the second CAR is or contains a cytoplasmic signaling domain of a zeta chain of a CD3-zeta (CD3ζ) chain or a functional variant or signaling portion thereof, optionally a human CD3 zeta chain. In some of any of the provided embodiments of engineered cells, the intracellular signaling region of the second CAR contains the sequence set forth in SEQ ID NO:20 or a sequence of amino acids that has at least 90%, 91%, 92%, 93%, 94%, 95%, 96%, 97%, 98%, or 99% sequence identity to SEQ ID NO:20.

In some of any of the provided embodiments of engineered cells, the intracellular signaling region of the second CAR further contains a costimulatory signaling region. In some of any of the provided embodiments of engineered cells, the costimulatory signaling region of the second CAR contains an intracellular signaling domain of a T cell costimulatory molecule or a signaling portion thereof. In some of any of the provided embodiments of engineered cells, the costimulatory signaling region of the second CAR contains an intracellular signaling domain of a CD28, a 4-1BB or an ICOS or a signaling portion thereof, optionally a human CD28, a human 4-1BB, or a human ICOS. In some of any of the provided embodiments of engineered cells, the costimulatory signaling region of the second CAR contains an intracellular signaling domain of 4-1BB or a signaling portion thereof. In some of any of the provided embodiments, at least one of the first chimeric antigen receptor and the second chimeric antigen receptor contains an intracellular signaling region containing an intracellular signaling domain of 4-1BB or a signaling portion thereof, optionally of human 4-1BB.

In some of any of the provided embodiments of engineered cells, the costimulatory signaling region of the second CAR contains: an intracellular signaling domain of a human CD28; and/or the sequence set forth in SEQ ID NO:46 or a sequence of amino acids that exhibits at least 90%, 91%, 92%, 93%, 94%, 95%, 96%, 97%, 98%, or 99% sequence identity to the sequence set forth in SEQ ID NO: 46.

In some of any of the provided embodiments of engineered cells, the costimulatory signaling region of the second CAR contains: an intracellular signaling domain of a human 4-1BB; and/or the sequence set forth in SEQ ID NO:19 or a sequence of amino acids that exhibits at least 90%, 91%, 92%, 93%, 94%, 95%, 96%, 97%, 98%, or 99% sequence identity to the sequence set forth in SEQ ID NO: 19.

In some of any of the provided embodiments of engineered cells, the encoded second chimeric antigen receptor contains from its N to C terminus in order: the antigen-binding domain, the spacer, the transmembrane domain and the intracellular signaling region.

In some of any of the provided embodiments, the engineered cell is a lymphocyte. In some of any of the provided embodiments, the engineered cell is an NK cell or a T cell. In some of any of the provided embodiments, the engineered cell is a T cell and the T cell is a CD4+ or a CD8+ T cell.

In some of any of the provided embodiments, the engineered cell was engineered from a primary cell obtained from a subject.

In some of any of the provided embodiments, the engineered cell is among a plurality of the engineered cells, where less than or less than about 10%, 9%, 8%, 7%, 5%, 4%, 3%, 2% or 1% of the cells in the plurality contain a chimeric antigen receptor that exhibits tonic signaling and/or antigen-independent activity or signaling.

Also provided are compositions containing any of the chimeric receptors provided herein. Also provided are compositions containing any of the engineered cells provided herein. In some of any of the provided embodiments, the composition contains CD4+ and CD8+ T cells and the ratio of CD4+ to CD8+ T cells is from or from about 1:3 to 3:1, optionally 1:2 to 2:1. In some of any of the provided embodiments, the composition contains CD4+ and CD8+ T cells and the ratio of CD4+ to CD8+ T cells is about 1:1.

Also provided are compositions containing: a plurality of first engineered cells containing a first chimeric antigen receptor that is any of the chimeric antigen receptors provided herein or encoded by any of the polynucleotides provided herein; and a plurality of second engineered cells containing a second chimeric antigen receptor. In some of any of the provided embodiments, among a plurality of the first engineered cells, less than or less than about 10%, 9%, 8%, 7%, 5%, 4%, 3%, 2% or 1% of the cells in the plurality contain a chimeric antigen receptor that exhibits tonic signaling and/or antigen independent activity or signaling. In some of any of the provided embodiments, among a plurality of the second engineered cells, less than or less than about 10%, 9%, 8%, 7%, 5%, 4%, 3%, 2% or 1% of the cells in the plurality contain a chimeric antigen receptor that exhibits tonic signaling and/or antigen independent activity or signaling.

In some of any of the provided embodiments, the second chimeric receptor in the plurality of second engineered cells in the composition contains an extracellular antigen binding domain that specifically binds a second antigen expressed on or associated with multiple myeloma. In some of any of the provided embodiments, the second CAR in the plurality of second engineered cells in the composition contains the extracellular antigen binding domain that binds the second antigen, a spacer, a transmembrane domain, and an intracellular signaling region. In some of any of the provided embodiments, the second antigen in the plurality of second engineered cells in the composition is selected from B cell maturation antigen (BCMA), CD38, CD138, CS-1, BAFF-R, TACI or FcRH5. In some of any of the provided embodiments, the second antigen in the plurality of second engineered cells in the composition is BCMA.

In some of any of the provided embodiments, the second CAR in the plurality of second engineered cells in the composition contains: (1) an extracellular antigen-binding domain that specifically binds BCMA, wherein the extracellular antigen-binding domain contains: (i) a variable heavy chain (V_{H}) containing an amino acid sequence having at least 90%, 91%, 92%, 93%, 94%, 95%, 96%, 97%, 98%, or 99% sequence identity to the V_{H} region amino acid sequence set forth in any of SEQ ID NOs: 189, 191, 193, 195 or 197; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO: 190, 192, 194, 196 or 198; (2) a spacer; (3) a transmembrane; and (4) an intracellular signaling region. In some of such embodiments, the V_{H} region contains a CDR-H1, CDR-H2 and CDR-H3 contained within the V_{H} region amino acid sequence set forth in any of SEQ ID NOs: 189, 191, 193, 195 or 197; and the V_{L} region contains a CDR-L1, CDR-L2 and CDR-L3 contained within the V_{L} region amino acid sequence set forth in any of SEQ ID NOs: 190, 192, 194, 196 or 198.

In some of any of the provided embodiments, the second CAR in the plurality of second engineered cells in the composition contains: (1) an extracellular antigen-binding domain that specifically binds BCMA, wherein the extracellular antigen-binding domain contains: (i) a variable heavy chain (V_{H}) containing a heavy chain complementarity determining region 1 (CDR-H1) containing the amino acid sequence selected from any one of SEQ ID NOs: 199, 202, 206, 209, 212 or 215; (b) a heavy chain complementarity determining region 2 (CDR-H2) containing the amino acid sequence selected from any one of SEQ ID NOs: 200, 203, 207, 210, 213 or 216; and (c) a heavy chain complementarity determining region 3 (CDR-H3) containing the amino acid sequence selected from any one of SEQ ID NOs: 201, 204, 205, 208, 211, 214 or 217; and (ii) a variable light chain (V_{L}) region containing a light chain complementarity determining region 1 (CDR-L1) containing the amino acid sequence selected from any one of SEQ ID NOs: 218, 221, 224, 227, 230, 233 or 235; (b) a light chain complementarity determining region 2 (CDR-L2) containing the amino acid sequence selected from any one of SEQ ID NOs: 219, 222, 225, 228, 231, 234 or 236; and (c) a light chain complementarity determining region 3 (CDR-L3) containing the amino acid sequence selected from any one of SEQ ID NOs: 220, 223, 226, 229 or 232; (2) a spacer; (3) a transmembrane domain; and (4) an intracellular signaling region.

In some of any of the provided embodiments, the V_{H} region of the second CAR of the composition contains a CDR-H1, CDR-H2, and CDR-H3 containing the amino acid sequence of SEQ ID NOS:199, 200 and 201, respectively, and the V_{L} region of the second CAR contains a CDR-L1, CDR-L2, and CDR-L3 containing the amino acid sequence of SEQ ID NOS:218, 219 and 220, respectively; the V_{H} region of the second CAR contains a CDR-H1, CDR-H2, and CDR-H3 containing the amino acid sequence of SEQ ID NOS:202, 203, 204, respectively, and the V_{L} region of the second CAR contains a CDR-L1, CDR-L2, and CDR-L3 containing the amino acid sequence of SEQ ID NOS:221, 222 and 223, respectively; the V_{H} region of the second CAR contains a CDR-H1, CDR-H2, and CDR-H3 containing the amino acid sequence of SEQ ID NOS:199, 200, 205, respectively, and the V_{L} region of the second CAR contains a CDR-L1, CDR-L2, and CDR-L3 containing the amino acid sequence of SEQ ID NOS:224, 225, and 226, respectively; the V_{H} region of the second CAR contains a CDR-H1, CDR-H2, and CDR-H3 containing the amino acid sequence of SEQ ID NOS:206, 207, 208, respectively, and the V_{L} region of the second CAR contains a CDR-L1, CDR-L2, and CDR-L3 containing the amino acid sequence of SEQ ID NOS:227, 228 and 229, respectively; the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; the V_{H} region of the second CAR contains a CDR-H1, CDR-H2, and CDR-H3 containing the amino acid sequence of SEQ ID NOS:212, 213 and 214, respectively, and the V_{L} region of the second CAR contains a CDR-L1, CDR-L2, and CDR-L3 containing the amino acid sequence of SEQ ID NOS:233, 234 and 229, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively.

In some of any of the provided embodiments, the V_{H} region of the second CAR in the composition contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively.

In some of any of the provided embodiments, the V_{H} region and V_{L} region of the second CAR in the composition contains the amino acid sequences set forth in SEQ ID NO:189 and SEQ ID NO:190, respectively, or a sequence of amino acids that exhibits at least 90%, 91%, 92%, 93%, 94%, 95%, 96%, 97%, 98%, or 99% sequence identity to SEQ ID NO:189 and SEQ ID NO:190; the V_{H} region and V_{L} region of the second CAR contains the amino acid sequences set forth in SEQ ID NO:191 and SEQ ID NO:192, respectively, or a sequence of amino acids that exhibits at least 90%, 91%, 92%, 93%, 94%, 95%, 96%, 97%, 98%, or 99% sequence identity to SEQ ID NO:191 and SEQ ID NO:192; the V_{H} region and V_{L} region of the second CAR contains the amino acid sequences set forth in SEQ ID NO:193 and SEQ ID NO:194, respectively, or a sequence of amino acids that exhibits at least 90%, 91%, 92%, 93%, 94%, 95%, 96%, 97%, 98%, or 99% sequence identity to SEQ ID NO:193 and SEQ ID NO:194; the V_{H} region and V_{L} region of the second CAR contains the amino acid sequences set forth in SEQ ID NO:195 and SEQ ID NO:196, respectively, or a sequence of amino acids that exhibits at least 90%, 91%, 92%, 93%, 94%, 95%, 96%, 97%, 98%, or 99% sequence identity to SEQ ID NO:195 and SEQ ID NO:196; or the V_{H} region and V_{L} region of the second CAR contains the amino acid sequences set forth in SEQ ID NO:197 and SEQ ID NO:198, respectively, or a sequence of amino acids that exhibits at least 90%, 91%, 92%, 93%, 94%, 95%, 96%, 97%, 98%, or 99% sequence identity to SEQ ID NO:197 and SEQ ID NO:198.

In some of any of the provided embodiments, the V_{H} region and V_{L} region of the second CAR in the composition contains the amino acid sequences set forth in SEQ ID NO:189 and SEQ ID NO:190, respectively; the V_{H} region and V_{L} region of the second CAR in the composition contains the amino acid sequences set forth in SEQ ID NO:191 and SEQ ID NO:192; the V_{H} region and V_{L} region of the second CAR in the composition contains the amino acid sequences set forth in SEQ ID NO:193 and SEQ ID NO:194; the V_{H} region and V_{L} region of the second CAR in the composition contains the amino acid sequences set forth in SEQ ID NO:195 and SEQ ID NO:196; or the V_{H} region and V_{L} region of the second CAR in the composition contains the amino acid sequences set forth in SEQ ID NO:197 and SEQ ID NO:198, respectively.

In some of any of the provided embodiments, the extracellular antigen-binding domain of the second CAR in the composition is a single chain antibody fragment. In some of such embodiments, the fragment is or contains a single chain variable fragment (scFv). In some of any of the provided embodiments, the V_{H} region and the V_{L} region in the second CAR in the composition are joined by a flexible linker. In some of any of the provided embodiments, the linker in the second CAR in the composition contains the amino acid sequence GGGGSGGGGSGGGGS (SEQ ID NO:52). In some of any of the provided embodiments, the linker in the second CAR in the composition contains the amino acid sequence GGGGSGGGGSGGGGSGGGGS (SEQ ID NO:320).

In some of any of the provided embodiments, the V_{H} region is amino-terminal to the V_{L} region in the second CAR in the composition. In some of any of the provided embodiments, the V_{H} region is carboxy-terminal to the V_{L} region in the second CAR in the composition. In some of any of the provided embodiments, the antigen-binding domain in the second CAR in the composition contains the amino acid sequence selected from any one of SEQ ID NOs: 227, 238, 239, 240 or 241 or an amino acid sequence having at least 90%, 91%, 92%, 93%, 94%, 95%, 96%, 97%, 98%, or 99% sequence identity to the amino acid sequence selected from any one of SEQ ID NOs: 227, 238, 239, 240 or 241. In some of any of the provided embodiments, the antigen-binding domain in the second CAR in the composition contains the amino acid sequence selected from any one of SEQ ID NOs: 227, 238, 239, 240 or 241.

In some of any of the provided embodiments, the V_{H} region of the second CAR in the composition contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively; and/or the V_{H} region and V_{L} region of the second CAR in the composition contains the amino acid sequences set forth in SEQ ID NO:197 and SEQ ID NO:198, respectively; and/or the antigen-binding domain of the second CAR in the composition contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241. In some of any of the provided embodiments, the V_{H} region of the second CAR in the composition contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; and/or the V_{H} region and V_{L} region of the second CAR in the composition contains the amino acid sequences set forth in SEQ ID NO:197 and SEQ ID NO:198, respectively; and/or the antigen-binding domain of the second CAR in the composition contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241.

In some of any of the provided embodiments, the transmembrane domain of the second CAR in the composition is or contains a transmembrane domain derived from CD4, CD28, or CD8, optionally from a human CD4, a human CD28 or a human CD8. In some of any of the provided embodiments, the transmembrane domain of the second CAR in the composition is or contains a transmembrane domain derived from a human CD28; and/or the transmembrane domain of the second CAR in the composition is or contains the sequence set forth in SEQ ID NO:18 or a sequence of amino acids that exhibits at least 90%, 91%, 92%, 93%, 94%, 95%, 96%, 97%, 98%, or 99% sequence identity to SEQ ID NO:18. In some of any of the provided embodiments, the transmembrane domain of the second CAR in the composition is or contains the sequence set forth in SEQ ID NO:18.

In some of any of the provided embodiments, the intracellular signaling region of the second CAR in the composition contains an intracellular signaling domain. In some of any of the provided embodiments, the intracellular signaling domain of the second CAR in the composition is capable of inducing a primary activation signal in a T cell, is a T cell receptor (TCR) component and/or contains an immunoreceptor tyrosine-based activation motif (ITAM). In some of any of the provided embodiments, the intracellular signaling domain of the second CAR in the composition is or contains a cytoplasmic signaling domain of a zeta chain of a CD3-zeta (CD3ζ) chain or a functional variant or signaling portion thereof, optionally a human CD3 zeta chain. In some of any of the provided embodiments, the intracellular signaling region of the second CAR in the composition contains the sequence set forth in SEQ ID NO:20 or a sequence of amino acids that has at least 90%, 91%, 92%, 93%, 94%, 95%, 96%, 97%, 98%, or 99% sequence identity to SEQ ID NO:20.

In some of any of the provided embodiments, the intracellular signaling region of the second CAR in the composition further contains a costimulatory signaling region. In some of any of the provided embodiments, the costimulatory signaling region of the second CAR in the composition contains an intracellular signaling domain of a T cell costimulatory molecule or a signaling portion thereof. In some of any of the provided embodiments, the costimulatory signaling region of the second CAR in the composition contains an intracellular signaling domain of a CD28, a 4-1BB or an ICOS or a signaling portion thereof, optionally a human CD28, a human 4-1BB, or a human ICOS. In some of any of the provided embodiments, the costimulatory signaling region of the second CAR in the composition contains an intracellular signaling domain of a 4-1BB or a signaling portion thereof, optionally a human 4-1BB. In some of any of the provided embodiments, at least one of the first chimeric antigen receptor and the second chimeric antigen receptor contains an intracellular signaling region containing an intracellular signaling domain of 4-1BB or a signaling portion thereof, optionally of human 4-1BB. In some of any of the provided embodiments, the costimulatory signaling region of the second CAR in the composition contains: an intracellular signaling domain of a human CD28; and/or the sequence set forth in SEQ ID NO:46 or a sequence of amino acids that exhibits at least 90%, 91%, 92%, 93%, 94%, 95%, 96%, 97%, 98%, or 99% sequence identity to the sequence set forth in SEQ ID NO: 46. In some of any of the provided embodiments, the costimulatory signaling region of the second CAR in the composition contains: an intracellular signaling domain of a human 4-1BB; and/or the sequence set forth in SEQ ID NO:19 or a sequence of amino acids that exhibits at least 90%, 91%, 92%, 93%, 94%, 95%, 96%, 97%, 98%, or 99% sequence identity to the sequence set forth in SEQ ID NO: 19.

In some of any of the provided embodiments, the encoded second chimeric antigen receptor of the composition contains from its N to C terminus in order: the antigen-binding domain, the spacer, the transmembrane domain and the intracellular signaling region.

In some of any of the provided embodiments, the plurality of first engineered cells in the composition contains T cells, optionally wherein the T cells include CD4+ and CD8+ T cells, optionally wherein the ratio of CD4+ to CD8+ T cells is from or from about 1:3 to 3:1, optionally 1:2 to 2:1. In some of any of the provided embodiments, the plurality of first engineered cells in the composition contains T cells, optionally wherein the T cells include CD4+ and CD8+ T cells, optionally wherein the ratio of CD4+ to CD8+ T cells is about 1:1.

In some of any of the provided embodiments, the plurality of second engineered cells in the composition contains T cells, optionally wherein the T cells include CD4+ and CD8+ T cells, optionally wherein the ratio of CD4+ to CD8+ T cells is from or from about 1:3 to 3:1, optionally 1:2 to 2:1. In some of any of the provided embodiments, the plurality of second engineered cells in the composition contains T cells, optionally wherein the T cells include CD4+ and CD8+ T cells, optionally wherein the ratio of CD4+ to CD8+ T cells is about 1:1.

In some of any of the provided embodiments, the composition contains a ratio of the first plurality of engineered cells and the second plurality of engineered cells that is from or from about 1:3 to 3:1, optionally 1:2 to 2:1, optionally is or is about 1:1. In some of any of the provided embodiments, the composition contains the first plurality of cells expressing the first chimeric antigen receptor and the second plurality of cells expressing the second chimeric antigen receptor at a ratio that is from about 1:3 to 3:1, optionally about 1:2 to 2:1. In particular embodiments, the ratio of the first plurality of engineered cells and the second plurality of engineered cells in the composition is or is about 1:1. In some of any of the provided embodiments, the composition further contains a pharmaceutically acceptable excipient. In some of any of the provided embodiments, the composition is sterile.

Also provided herein are uses of any of the compositions provided herein. In some of any of the provided embodiments, the composition is for use in treating a subject with a disease or condition. In some of any of the provided embodiments, the disease or condition is a cancer. In some of any of the provided embodiments, the disease or condition is multiple myeloma, optionally relapsed/refractory multiple myeloma. The provided uses and compositions for use provided herein can be for treating a subject in accord with aspects of any of the provided methods.

Also provided herein are methods of treatment, containing administering any of the compositions provided herein containing any of the engineered cells provided herein or any of the compositions provided herein containing any of the chimeric antigen receptors provided herein to a subject having a disease or disorder. In some of any of the provided embodiments, the dose of cells contains between at or about 1.0×10⁷ CAR-expressing T cells and 1.2×10⁹ CAR-expressing T cells, between about 1.25×10⁷ CAR-expressing T cells and 1.2×10⁹ CAR-expressing T cells, between about 1.5×10⁷ CAR-expressing T cells and 1.2×10⁹ CAR-expressing T cells, between about 5.0×10⁷ CAR-expressing T cells and 4.5×10⁸ CAR-expressing T cells, between about 1.5×10⁸ CAR-expressing T cells and 3.0×10⁸ CAR-expressing T cells. In some of any of the provided embodiments, the dose of cells contains between at or about 2.5×10⁷ CAR-expressing T cells and 1.2×10⁹ CAR-expressing T cells, between at or about 5.0×10⁷ CAR-expressing T cells and 4.5×10⁸ CAR-expressing T cells, between at or about 1.5×10⁸ CAR-expressing T cells and 3.0×10⁸ CAR-expressing T cells.

In some of any embodiments, the dose of cells contains between at or about 1×10⁷ CAR-expressing T cells and at or about 2×10⁹ CAR-expressing T cells. In some of any embodiments, the dose of cells contains between at or about 2.5×10⁷ CAR-expressing T cells and at or about 1.2×10⁹ CAR-expressing T cells, between at or about 5.0×10⁸ CAR-expressing T cells and at or about 4.5×10⁸ CAR-expressing T cells, or between at or about 1.5×10⁸ CAR-expressing T cells and at or about 3.0×10⁸ CAR-expressing T cells. In some of any embodiments, the dose of cells contains at or about 1.0×10⁷, at or about 1.5×10⁷, at or about 2.5×10⁷, at or about 5.0×10⁷, at or about 7.5×10⁷, at or about 1.5×10⁸, at or about 2.25×10⁸, at or about 3.0×10⁸, at or about 4.5×10⁸, at or about 6.0×10⁸, at or about 8.0×10⁸, or at or about 1.2×10⁹ CAR-expressing T cells. In some of any embodiments, the dose of cells contains at or about 5.0×10⁷, at or about 1.5×10⁸, at or about 3.0×10⁸ or at or about 4.5×10⁸ CAR-expressing T cells. In some of any embodiments, the dose of cells contains at or about 5.0×10⁷, at or about 1.5×10⁸, at or about 3.0×10⁸ or at or about 4.5×10⁸ CAR-expressing T cells. In some of any embodiments, the dose of cells contains at or about 5.0×10⁷ CAR-expressing T cells.

Also provided herein are uses of a first composition containing a plurality of first engineered cells containing a first chimeric antigen receptor that is any chimeric antigen receptor provided herein or encoded by any of the polynucleotides provided herein and a second composition containing a plurality of second engineered cells containing a second chimeric antigen receptor. In some of any of the provided embodiments, the compositions are used together for use in treating a subject with a disease or condition. In some of any of the provided embodiments, the disease or condition is a cancer. In some of any of the provided embodiments, the disease or condition is multiple myeloma, optionally relapsed/refractory multiple myeloma. The provided uses and compositions for use provided herein can be for treating a subject in accord with aspects of any of the provided methods.

Also provided here in are methods of treatment, that include: administering a composition containing a plurality of first engineered cells containing a first chimeric antigen receptor that is any chimeric antigen receptor provided herein or encoded by any of the polynucleotides provided herein to a subject having a disease or disorder; and administering to the subject a composition containing a plurality of second engineered cells containing a second chimeric antigen receptor. In some of any of the provided embodiments, the dose of the plurality of first engineered cells and the dose of the plurality of second engineered cells independently contain between at or about 1.0×10⁷ CAR-expressing T cells and 1.5×10⁹ CAR-expressing T cells, between at or about 1.25×10⁷ CAR-expressing T cells and 0.6×10⁸ CAR-expressing T cells, between at or about 2.5×10⁷ CAR-expressing T cells and 2.25×10⁸ CAR-expressing T cells, between at or about 7.5×10⁷ CAR-expressing T cells and 1.5×10⁸ CAR-expressing T cells, between at or about 2.5×10⁷ CAR-expressing T cells and 1.2×10⁹ CAR-expressing T cells, between at or about 5.0×10⁷ CAR-expressing T cells and 4.5×10⁸ CAR-expressing T cells, between at or about 1.5×10⁸ CAR-expressing T cells and 3.0×10⁸ CAR-expressing T cells. In some of any embodiments, the dose of the plurality of first engineered cells and the dose of the plurality of second engineered cells independently contain between at or about 1×10⁷ CAR-expressing T cells and at or about 2×10⁹ CAR-expressing T cells. In some of any embodiments, the dose of cells contains between at or about 2.5×10⁷ CAR-expressing T cells and at or about 1.2×10⁹ CAR-expressing T cells, between at or about 5.0×10⁷ CAR-expressing T cells and at or about 4.5×10⁸ CAR-expressing T cells, or between at or about 1.5×10⁸ CAR-expressing T cells and at or about 3.0×10⁸ CAR-expressing T cells. In some of any embodiments, the dose of cells contains at or about 1.5×10⁷, at or about 2.5×10⁷, at or about 5.0×10⁷, at or about 7.5×10⁷, at or about 1.5×10⁸, at or about 2.25×10⁸, at or about 3.0×10⁸, at or about 4.5×10⁸, at or about 6.0×10⁸, at or about 8.0×10⁸, or at or about 1.2×10⁹ CAR-expressing T cells. In some of any embodiments, the dose of cells contains at or about 5.0×10⁷, at or about 1.5×10⁸, at or about 3.0×10⁸ or at or about 4.5×10⁸ CAR-expressing T cells. In some of any embodiments, the dose of cells contains at or about 5.0×10⁷, at or about 1.5×10⁸, at or about 3.0×10⁸ or at or about 4.5×10⁸ CAR-expressing T cells. In some of any embodiments, the dose of cells contains at or about 5.0×10⁷ CAR-expressing T cells.

In some of any of the provided embodiments, the composition containing the plurality of first engineered cells and the composition containing the plurality of second engineered cells are administered simultaneously, sequentially or intermittently. In some of any of the provided embodiments, the composition containing the plurality of first engineered cells and the composition containing the plurality of second engineered cells are administered sequentially in any order.

In some of any of the provided embodiments, among a plurality of the first engineered cells of compositions in the provided methods of treatment or for uses in treating, less than or less than about 10%, 9%, 8%, 7%, 5%, 4%, 3%, 2% or 1% of the cells in the plurality contain a chimeric antigen receptor that exhibits tonic signaling and/or antigen independent activity or signaling.

In some of any of the provided embodiments, among a plurality of the second engineered cells of compositions in the provided methods of treatment or for uses in treating, less than or less than about 10%, 9%, 8%, 7%, 5%, 4%, 3%, 2% or 1% of the cells in the plurality contain a chimeric antigen receptor that exhibits tonic signaling and/or antigen independent activity or signaling.

In some of any of the provided embodiments, the second chimeric receptor in engineered cells of compositions in the methods of treatment or for uses in treating contains an extracellular antigen binding domain that specifically binds a second antigen expressed on or associated with multiple myeloma.

In some of any of the provided embodiments, the second CAR in engineered cells of compositions in the provided methods or for uses in treating contains the extracellular antigen binding domain that binds the second antigen, a spacer, a transmembrane domain, and an intracellular signaling region.

In some of any of the provided embodiments, the second antigen targeted by the second CAR in engineered cells of compositions in the provided methods or for uses in treating contains is selected from B cell maturation antigen (BCMA), CD38, CD138, CS-1, BAFF-R, TACI or FcRH5. In some of any of the provided embodiments, the second antigen in the provided methods contains is BCMA.

In some of any of the provided embodiments, the second CAR in engineered cells of compositions in the provided methods or for uses in treating contains: (1) an extracellular antigen-binding domain that specifically binds BCMA, wherein the extracellular antigen-binding domain contains: (i) a variable heavy chain (V_{H}) containing an amino acid sequence having at least 90%, 91%, 92%, 93%, 94%, 95%, 96%, 97%, 98%, or 99% sequence identity to the V_{H} region amino acid sequence set forth in any of SEQ ID NOs: 189, 191, 193, 195 or 197; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in any of SEQ ID NO: 190, 192, 194, 196 or 198; (2) a spacer; (3) a transmembrane; and (4) an intracellular signaling region. In some of any of the provided embodiments, the V_{H} region of the second CAR in the provided methods contains a CDR-H1, CDR-H2 and CDR-H3 contained within the V_{H} region amino acid sequence set forth in any of SEQ ID NOs: 189, 191, 193, 195 or 197; and the V_{L} region contains a CDR-L1, CDR-L2 and CDR-L3 contained within the V_{L} region amino acid sequence set forth in any of SEQ ID NOs: 190, 192, 194, 196 or 198.

In some of any of the provided embodiments, the second CAR in engineered cells of compositions in the provided methods or for uses in treating contains: (1) an extracellular antigen-binding domain that specifically binds BCMA, wherein the extracellular antigen-binding domain contains: (i) a variable heavy chain (V_{H}) containing an amino acid sequence having at least 90%, 91%, 92%, 93%, 94%, 95%, 96%, 97%, 98%, or 99% sequence identity to the V_{H} region amino acid sequence set forth in SEQ ID NO: 197; and (ii) a variable light chain (V_{L}) region containing an amino acid sequence having at least 90%, 91%, 92%, 93%, 94%, 95%, 96%, 97%, 98%, or 99% sequence identity to the V_{L} region amino acid sequence set forth in SEQ ID NO: 198; (2) a spacer; (3) a transmembrane; and (4) an intracellular signaling region. In some of any of the provided embodiments, the V_{H} region of the second CAR in the provided methods contains a CDR-H1, CDR-H2 and CDR-H3 contained within the V_{H} region amino acid sequence set forth in SEQ ID NO: 197; and the V_{L} region contains a CDR-L1, CDR-L2 and CDR-L3 contained within the V_{L} region amino acid sequence set forth in SEQ ID NO: 198.

In some of any of the provided embodiments, the second CAR in engineered cells of compositions in the provided methods or for uses in treating contains: (1) an extracellular antigen-binding domain that specifically binds BCMA, wherein the extracellular antigen-binding domain contains: (i) a variable heavy chain (V_{H}) containing a heavy chain complementarity determining region 1 (CDR-H1) containing the amino acid sequence selected from any one of SEQ ID NOs: 199, 202, 206, 209, 212 or 215; (b) a heavy chain complementarity determining region 2 (CDR-H2) containing the amino acid sequence selected from any one of SEQ ID NOs: 200, 203, 207, 210, 213 or 216; and (c) a heavy chain complementarity determining region 3 (CDR-H3) containing the amino acid sequence selected from any one of SEQ ID NOs: 201, 204, 205, 208, 211, 214 or 217; and (ii) a variable light chain (V_{L}) region containing a light chain complementarity determining region 1 (CDR-L1) containing the amino acid sequence selected from any one of SEQ ID NOs: 218, 221, 224, 227, 230, 233 or 235; (b) a light chain complementarity determining region 2 (CDR-L2) containing the amino acid sequence selected from any one of SEQ ID NOs: 219, 222, 225, 228, 231, 234 or 236; and (c) a light chain complementarity determining region 3 (CDR-L3) containing the amino acid sequence selected from any one of SEQ ID NOs: 220, 223, 226, 229 or 232; (2) a spacer; (3) a transmembrane domain; and (4) an intracellular signaling region.

In some of any of the provided embodiments, the second CAR in engineered cells of compositions in the provided methods or for uses in treating contains an extracellular antigen-binding domain in which the V_{H} region of the second CAR contains a CDR-H1, CDR-H2, and CDR-H3 containing the amino acid sequence of SEQ ID NOS:199, 200 and 201, respectively, and the V_{L} region of the second CAR contains a CDR-L1, CDR-L2, and CDR-L3 containing the amino acid sequence of SEQ ID NOS:218, 219 and 220, respectively; the V_{H} region of the second CAR contains a CDR-H1, CDR-H2, and CDR-H3 containing the amino acid sequence of SEQ ID NOS:202, 203, 204, respectively, and the V_{L} region of the second CAR contains a CDR-L1, CDR-L2, and CDR-L3 containing the amino acid sequence of SEQ ID NOS:221, 222 and 223, respectively; the V_{H} region of the second CAR contains a CDR-H1, CDR-H2, and CDR-H3 containing the amino acid sequence of SEQ ID NOS:199, 200, 205, respectively, and the V_{L} region of the second CAR contains a CDR-L1, CDR-L2, and CDR-L3 containing the amino acid sequence of SEQ ID NOS:224, 225, and 226, respectively; the V_{H} region of the second CAR contains a CDR-H1, CDR-H2, and CDR-H3 containing the amino acid sequence of SEQ ID NOS:206, 207, 208, respectively, and the V_{L} region of the second CAR contains a CDR-L1, CDR-L2, and CDR-L3 containing the amino acid sequence of SEQ ID NOS:227, 228 and 229, respectively; the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; the V_{H} region of the second CAR contains a CDR-H1, CDR-H2, and CDR-H3 containing the amino acid sequence of SEQ ID NOS:212, 213 and 214, respectively, and the V_{L} region of the second CAR contains a CDR-L1, CDR-L2, and CDR-L3 containing the amino acid sequence of SEQ ID NOS:233, 234 and 229, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively.

In some of any of the provided embodiments, the second CAR in engineered cells of compositions in the provided methods or for uses in treating contains an extracellular antigen-binding domain in which the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; or the V_{H} region of the second CAR in the provided methods contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively. In some of any of the provided embodiments, the second CAR in engineered cells of compositions in the provided methods or for uses in treating contains an extracellular antigen-binding domain in which the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively.

In some of any of the provided embodiments, the second CAR in engineered cells of compositions in the provided methods or for uses in treating contains an extracellular antigen-binding domain in which the V_{H} region and VL region contains the amino acid sequences set forth in SEQ ID NO:189 and SEQ ID NO:190, respectively, or a sequence of amino acids that exhibits at least 90%, 91%, 92%, 93%, 94%, 95%, 96%, 97%, 98%, or 99% sequence identity to SEQ ID NO:189 and SEQ ID NO:190; the V_{H} region and VL region of the second CAR contains the amino acid sequences set forth in SEQ ID NO:191 and SEQ ID NO:192, respectively, or a sequence of amino acids that exhibits at least 90%, 91%, 92%, 93%, 94%, 95%, 96%, 97%, 98%, or 99% sequence identity to SEQ ID NO:191 and SEQ ID NO:192; the V_{H} region and VL region of the second CAR contains the amino acid sequences set forth in SEQ ID NO:193 and SEQ ID NO:194, respectively, or a sequence of amino acids that exhibits at least 90%, 91%, 92%, 93%, 94%, 95%, 96%, 97%, 98%, or 99% sequence identity to SEQ ID NO:193 and SEQ ID NO:194; the V_{H} region and VL region of the second CAR contains the amino acid sequences set forth in SEQ ID NO:195 and SEQ ID NO:196, respectively, or a sequence of amino acids that exhibits at least 90%, 91%, 92%, 93%, 94%, 95%, 96%, 97%, 98%, or 99% sequence identity to SEQ ID NO:195 and SEQ ID NO:196; or the V_{H} region and VL region of the second CAR contains the amino acid sequences set forth in SEQ ID NO:197 and SEQ ID NO:198, respectively, or a sequence of amino acids that exhibits at least 90%, 91%, 92%, 93%, 94%, 95%, 96%, 97%, 98%, or 99% sequence identity to SEQ ID NO:197 and SEQ ID NO:198.

In some of any of the provided embodiments, the second CAR in engineered cells of compositions in the provided methods or for uses in treating contains an extracellular antigen-binding domain in which the V_{H} region and VL region contains the amino acid sequences set forth in SEQ ID NO:197 and SEQ ID NO:198, respectively, or a sequence of amino acids that exhibits at least 90%, 91%, 92%, 93%, 94%, 95%, 96%, 97%, 98%, or 99% sequence identity to SEQ ID NO:197 and SEQ ID NO:198.

In some of any of the provided embodiments, the second CAR in engineered cells of compositions in the provided methods or for uses in treating contains an extracellular antigen-binding domain in which the V_{H} region and VL region contains the amino acid sequences set forth in SEQ ID NO:189 and SEQ ID NO:190, respectively; the V_{H} region and VL region of the second CAR contains the amino acid sequences set forth in SEQ ID NO:191 and SEQ ID NO:192; the V_{H} region and VL region of the second CAR contains the amino acid sequences set forth in SEQ ID NO:193 and SEQ ID NO:194; the V_{H} region and VL region of the second CAR contains the amino acid sequences set forth in SEQ ID NO:195 and SEQ ID NO:196; or the V_{H} region and VL region of the second CAR contains the amino acid sequences set forth in SEQ ID NO:197 and SEQ ID NO:198, respectively. In some of any of the provided embodiments, the second CAR in engineered cells of compositions in the provided methods or for uses in treating contains an extracellular antigen-binding domain in which the V_{H} region and VL region contains the amino acid sequences set forth in SEQ ID NO:197 and SEQ ID NO:198, respectively.

In some of any of the provided embodiments, the extracellular antigen-binding domain of the second CAR in engineered cells in compositions in in the provided methods or for uses in treating is a single chain antibody fragment. In some of any of the provided embodiments, the fragment is or contains a single chain variable fragment (scFv).

In some of any of the provided embodiments of the provided methods or for uses in treating, the V_{H} region and the V_{L} region of the extracellular antigen-binding domain of the second CAR in engineered cells in compositions are joined by a flexible linker. In some of any of the provided embodiments, the linker of the second CAR in engineered cells in compositions contains the amino acid sequence GGGGSGGGGSGGGGS (SEQ ID NO:52). In some of any of the provided embodiments, the linker of the second CAR in engineered cells in compositions contains the amino acid sequence GGGGSGGGGSGGGGSGGGGS (SEQ ID NO:320).

In some of any of the provided embodiments of the provided methods or for uses in treating, the V_{H} region is amino-terminal to the V_{L} region in the extracellular antigen-binding domain of the second CAR. In some of any of the provided embodiments of the provided methods, the V_{H} region is carboxy-terminal to the V_{L} region in the second CAR.

In some of any of the provided embodiments of the provided methods or for uses in treating, the extracellular antigen-binding domain of the second CAR contains the amino acid sequence selected from any one of SEQ ID NOs: 227, 238, 239, 240 or 241 or an amino acid sequence having at least 90%, 91%, 92%, 93%, 94%, 95%, 96%, 97%, 98%, or 99% sequence identity to the amino acid sequence selected from any one of SEQ ID NOs: 227, 238, 239, 240 or 241. In some of any of the provided embodiments of the provided methods, the antigen-binding domain of the second CAR contains the amino acid sequence selected from any one of SEQ ID NOs: 227, 238, 239, 240 or 241. In some of any of the provided embodiments of the provided methods or for uses in treating, the extracellular antigen-binding domain of the second CAR contains the amino acid sequence set forth in SEQ ID NO: 241 or an amino acid sequence having at least 90%, 91%, 92%, 93%, 94%, 95%, 96%, 97%, 98%, or 99% sequence identity to the amino acid sequence set forth in SEQ ID NO:241. In some of any of the provided embodiments of the provided methods, the antigen-binding domain of the second CAR contains the amino acid sequence set forth in SEQ ID NO: 241.

In some of any of the provided embodiments of the provided methods or for uses in treating, the extracellular antigen-binding domain of the second CAR has a V_{H} region and a V_{L} region in which the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; or the V_{H} region of the second CAR contains a CDR-H1, CDR-H2, and CDR-H3 containing the amino acid sequence of SEQ ID NOS:215, 216 and 217, respectively, and the V_{L} region of the second CAR contains a CDR-L1, CDR-L2, and CDR-L3 containing the amino acid sequence of SEQ ID NOS:235, 236 and 232, respectively; and/or the V_{H} region and V_{L} region of the second CAR contains the amino acid sequences set forth in SEQ ID NO:197 and SEQ ID NO:198, respectively; and/or the antigen-binding domain of the second CAR contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241. In some of any of the provided embodiments of the provided methods or for uses in treating, the extracellular antigen-binding domain of the second CAR has a V_{H} region and a V_{L} region in which the V_{H} region of the second CAR contains a CDR-H1, CDR-H2, and CDR-H3 containing the amino acid sequence of SEQ ID NOS:209, 210 and 211, respectively, and the V_{L} region of the second CAR contains a CDR-L1, CDR-L2, and CDR-L3 containing the amino acid sequence of SEQ ID NOS:230, 231 and 232, respectively; and/or the V_{H} region and V_{L} region of the second CAR contains the amino acid sequences set forth in SEQ ID NO:197 and SEQ ID NO:198, respectively; and/or the antigen-binding domain of the second CAR contains the amino acid sequence set forth in SEQ ID NO: 241 or a sequence having at least 90%, 91%, 92%, 93%, 94%, 95%, 96%, 97%, 98%, or 99% sequence identity to the sequence set forth in SEQ ID NO:241.

In some of any of the provided embodiments of the provided methods or for uses in treating, the transmembrane domain of the second CAR is or contains a transmembrane domain derived from CD4, CD28, or CD8, optionally from a human CD4, a human CD28 or a human CD8. In some of any of the provided embodiments of the provided methods, the transmembrane domain of the second CAR is or contains a transmembrane domain derived from a human CD28; and/or the transmembrane domain is or contains the sequence set forth in SEQ ID NO:18 or a sequence of amino acids that exhibits at least 90%, 91%, 92%, 93%, 94%, 95%, 96%, 97%, 98%, or 99% sequence identity to SEQ ID NO:18. In some of any of the provided embodiments of the provided methods, the transmembrane domain of the second CAR is or contains the sequence set forth in SEQ ID NO:18.

In some of any of the provided embodiments of the provided methods or for uses in treating, the intracellular signaling region of the second CAR contains an intracellular signaling domain. In some of any of the provided embodiments of the provided methods, the intracellular signaling domain of the second CAR is capable of inducing a primary activation signal in a T cell, is a T cell receptor (TCR) component and/or contains an immunoreceptor tyrosine-based activation motif (ITAM). In some of any of the provided embodiments of the provided methods, the intracellular signaling domain of the second CAR is or contains a cytoplasmic signaling domain of a zeta chain of a CD3-zeta (CD3ζ) chain or a functional variant or signaling portion thereof, optionally a human CD3 zeta chain. In some of any of the provided embodiments of the provided methods, the intracellular signaling region of the second CAR contains the sequence set forth in SEQ ID NO:20 or a sequence of amino acids that has at least 90%, 91%, 92%, 93%, 94%, 95%, 96%, 97%, 98%, or 99% sequence identity to SEQ ID NO:20.

In some of any of the provided embodiments of the provided methods or for uses in treating, the intracellular signaling region of the second CAR further contains a costimulatory signaling region. In some of any of the provided embodiments of the provided methods, the costimulatory signaling region of the second CAR contains an intracellular signaling domain of a T cell costimulatory molecule or a signaling portion thereof. In some of any of the provided embodiments of the provided methods, the costimulatory signaling region of the second CAR contains an intracellular signaling domain of a CD28, a 4-1BB or an ICOS or a signaling portion thereof, optionally a human CD28, a human 4-1BB, or a human ICOS. In some of any of the provided embodiments of the provided methods, the costimulatory signaling region of the second CAR contains an intracellular signaling domain of a 4-1BB or a signaling portion thereof, optionally a human 4-1BB. In some of any of the provided embodiments, at least one of the first chimeric antigen receptor and the second chimeric antigen receptor contains an intracellular signaling region containing an intracellular signaling domain of 4-1BB or a signaling portion thereof, optionally of human 4-1BB.

In some of any of the provided embodiments of the provided methods or for uses in treating, the costimulatory signaling region of the second CAR contains: an intracellular signaling domain of a human CD28; and/or the sequence set forth in SEQ ID NO:46 or a sequence of amino acids that exhibits at least 90%, 91%, 92%, 93%, 94%, 95%, 96%, 97%, 98%, or 99% sequence identity to the sequence set forth in SEQ ID NO: 46.

In some of any of the provided embodiments of the provided methods or for uses in treating, the costimulatory signaling region of the second CAR contains: an intracellular signaling domain of a human 4-1BB; and/or the sequence set forth in SEQ ID NO:19 or a sequence of amino acids that exhibits at least 90%, 91%, 92%, 93%, 94%, 95%, 96%, 97%, 98%, or 99% sequence identity to the sequence set forth in SEQ ID NO: 19.

In some of any of the provided embodiments of the provided methods or for uses in treating, the encoded second chimeric antigen receptor contains from its N to C terminus in order: the antigen-binding domain, the spacer, the transmembrane domain and the intracellular signaling region.

Among polynucleotides provided herein are polynucleotides containing (i) a first nucleic acid sequence encoding a first chimeric antigen receptor (CAR) containing a first antigen binding domain; and (ii) a second nucleic acid sequence encoding a second chimeric antigen receptor (CAR) containing a second antigen binding domain; wherein the first CAR and second CAR each contain the following: (a) the first antigen binding domain or the second antigen binding domain, (b) a spacer, (c) a transmembrane domain, and (d) an intracellular signaling region comprising an intracellular signaling domain and a costimulatory signaling region; wherein one or more of (b) through (d) in the first CAR and the same one or more of (b) through (d) in the second CAR contains the identical amino acid sequence; and wherein the nucleotide sequence(s) encoding the one or more of (b) through (d) in the first CAR differs in sequence from the nucleotide sequence(s) encoding the same one or more of (b) through (d) in the second CAR.

Also provided here are polynucleotides containing (i) a first nucleic acid sequence encoding a first chimeric antigen receptor (CAR) containing a first antigen binding domain capable of binding to one of GPRC5D or BCMA and (ii) a second nucleic acid sequence encoding a second chimeric antigen receptor (CAR) containing a second antigen binding domain capable of binding to the other of GPRC5D or BCMA; wherein the first CAR and second CAR each contain the following: (a) the first antigen binding domain or the second antigen binding domain, (b) a spacer, (c) a transmembrane domain, and (d) an intracellular signaling region comprising an intracellular signaling domain and a costimulatory signaling region; wherein one or more of (b) through (d) in the first CAR and the same one or more of (b) through (d) in the second CAR contains the identical amino acid sequence; and wherein the nucleotide sequence(s) encoding the one or more of (b) through (d) in the first CAR differs in sequence from the nucleotide sequence(s) encoding the same one or more of (b) through (d) in the second CAR.

In some of any of the provided embodiments, the first and second antigen binding domains bind to the same antigen. In some of any of the provided embodiments, the first and second antigen binding domains bind different epitopes of the same antigen. In some of any of the provided embodiments, the first and second antigen binding domains bind to different antigens. In some of any of the provided embodiments, the first antigen binding domain binds a first antigen expressed by or associated with cells of a disease or condition and the second antigen binding domains binds a second antigen expressed by or associated with cells of the same disease or condition.

In some of any of the provided embodiments, the disease or condition is a cancer. In some of any of the provided embodiments, the disease or condition is a GPRC5D-expressing cancer. In some of any of the provided embodiments, the disease or condition is a BCMA-expressing cancer. In some of any of the provided embodiments, the disease or condition is a BCMA-expressing and GPRC5D-expressing cancer. In some of any of the provided embodiments, the cancer is a plasma cell malignancy and the plasma cell malignancy is multiple myeloma (MM) or plasmacytoma. In some of any of the provided embodiments, the cancer is multiple myeloma. In some of any of the provided embodiments, the cancer is relapsed/refractory multiple myeloma.

In some of any of the provided embodiments, the first and second antigen binding domain independently bind to an antigen selected from the group consisting of GPRC5D, BCMA, CD38, CD138, CS-1, BAFF-R, TACI and FcRH5. In some of any of the provided embodiments, the first antigen binding domain binds to B cell maturation antigen (BCMA). In some of any of the provided embodiments, the first antigen binding domain bind to G protein-coupled receptor class C group 5 member D (GPRC5D). In some of any of the provided embodiments, the second antigen binding domain binds to BCMA. In some of any of the provided embodiments, the second antigen binding domain binds to GPRC5D.

In some of any of the provided embodiments, (a) is or contains the first antigen binding domain or the second antigen binding domain, (b) is or contains a spacer, (c) is or contains a transmembrane domain, and (d) is or contains an intracellular signaling region containing an intracellular signaling domain and a costimulatory signaling region. In some of any of the provided embodiments, the one or more of (b) through (d) is one of (b) through (d). In some of any of the provided embodiments, the one or more of (b) through (d) is two of (b) through (d). In some of any of the provided embodiments, the one or more of (b) through (d) is each of (b) through (d).

In some of any of the provided embodiments, (a) is or contains the first antigen binding domain or the second antigen binding domain, (b) is or contains a spacer, (c) is or contains a transmembrane domain, and (d) is or contains an intracellular signaling region containing an intracellular signaling domain and a costimulatory signaling region. In some of any of the provided embodiments, the nucleotide sequence(s) encoding the one or more of (a) through (d) in the first CAR and the nucleotide sequence(s) encoding the same one or more of (a) through (d) in the second CAR comprises no more than about 20 consecutive base pairs of sequence homolog; and/or the first nucleic acid sequence encoding the first CAR and the second nucleic acid sequence encoding the second CAR contain no more than about 20 consecutive base pairs of sequence homology. In some of any of the provided embodiments, the nucleotide sequence(s) encoding the one or more of (a) through (d) in the first CAR and the nucleotide sequence(s) encoding the same one or more of (a) through (d) in the second CAR contains no more than between about 5 and about 15 consecutive base pairs of sequence homology; and/or the first nucleic acid sequence encoding the first CAR and the second nucleic acid sequence encoding the second CAR contain no more than about 5 and about 15 consecutive base pairs of sequence homology. In some of any of the provided embodiments, the nucleotide sequence(s) encoding the one or more of (a) through (d) in the first CAR and the nucleotide sequence(s) encoding the same one or more of (a) through (d) in the second CAR contain no more than about 10 consecutive base pairs of homology; and/or the first nucleic acid sequence encoding the first CAR and the second nucleic acid sequence encoding the second CAR contain no more than about 10 consecutive base pairs of sequence homology.

In some of any of the provided embodiments, the first nucleic acid encoding the first CAR and the second nucleic acid encoding the second CAR are separated by a nucleotide sequence encoding a multicistronic element, optionally wherein the multicistronic element is a bicistronic element. In some of any of the provided embodiments, the multicistronic element is an IRES or is a ribosome skip sequence or self-cleaving peptide. In some of any of the provided embodiments, the multicistronic element is a ribosome skip sequence or self-cleaving peptide and the ribosome skip sequence or self-cleaving peptide is a T2A, a P2A, an E2A, or an F2A element. In some of any of the provided embodiments, the nucleotide sequence encoding the one or more multicistronic element is codon diverged. In some of any of the provided embodiments, the nucleotide sequence encoding the T2A is codon diverged. In some of any of the provided embodiments, nucleotide sequence encoding the T2A is or contains the sequence set forth in SEQ ID NO:319.

In some of any of the provided embodiments, the first nucleic acid sequence encoding the first CAR is codon optimized for expression in a human cell. In some of any of the provided embodiments, the second nucleic acid sequence encoding the second CAR is codon optimized for expression in a human cell. In some of any of the provided embodiments, the polynucleotide is codon optimized for expression in a human cell. In some of any of the provided embodiments, following transcription of the polynucleotide in a human cell, optionally a human T cell, the transcribed mRNA, optionally messenger RNA, from the polynucleotide, exhibits at least about 70%, 75%, 80%, 85%, 90%, or 95% RNA homogeneity. In some of any of the provided embodiments, following transcription of the first nucleic acid encoding the first CAR of the polynucleotide in a human cell, optionally a human T cell, the transcribed mRNA, optionally messenger RNA, from the first nucleic acid exhibits at least about 70%, 75%, 80%, 85%, 90%, or 95% RNA homogeneity. In some of any of the provided embodiments, following transcription of the second nucleic acid encoding the second CAR of the polynucleotide in a human cell, optionally a human T cell, the transcribed mRNA, optionally messenger RNA, from the second nucleic acid exhibits at least about 70%, 75%, 80%, 85%, 90%, or 95% RNA homogeneity.

In some of any of the provided embodiments, any potential splice donor and/or splice acceptor site present in the first nucleic acid encoding the first CAR exhibits a splice prediction score of about or at least about less than 0.70, 0.65, 0.60, 0.55, 0.50, 0.45, 0.40, 0.35, 0.30, 0.25, 0.20 and/or is predicted to be involved in a splice event with a probability of less than 70%, less than 65%, less than 60%, less than 55%, less than 50%, less than 45%, less than 40%, less than 35%, less than 30%, less than 25%, or less than 20%. In some of any of the provided embodiments, any potential splice donor or acceptor site in the second nucleic acid encoding the second CAR exhibits a splice prediction score of about or at least about less than 0.70, 0.65, 0.60, 0.55, 0.50, 0.45, 0.40, 0.35, 0.30, 0.25, 0.20 and/or is predicted to be involved in a splice event with a probability of less than 70%, less than 65%, less than 60%, less than 55%, less than 50%, less than 45%, less than 40%, less than 35%, less than 30%, less than 25%, or less than 20%. In some of any of the provided embodiments, any potential splice donor or acceptor sites in the polynucleotide exhibits a splice prediction score of about or at least about less than 0.70, 0.65, 0.60, 0.55, 0.50, 0.45, 0.40, 0.35, 0.30, 0.25, 0.20 and/or is predicted to be involved in a splice event with a probability of less than 70%, less than 65%, less than 60%, less than 55%, less than 50%, less than 45%, less than 40%, less than 35%, less than 30%, less than 25%, or less than 20%.

In some of any of the provided embodiments, the first and/or second antigen binding domain of (a) is a single chain antibody fragment. In some of any of the provided embodiments, the first and/or second antigen binding domain of (a) is or contains a single chain variable fragment (scFv). In some of any of the provided embodiments, the first and/or second antigen binding domain of (a) contains a variable heavy chain (VH) region and a variable light chain (VL) region.

In some of any of the provided embodiments, the first antigen binding domain or the second antigen binding domain contains a VH region that contains a CDR-H1 as set forth in SEQ ID NO:209, a CDR-H2 as set forth in SEQ ID NO:210, and a CDR-H3 as set forth in SEQ ID NO:211 and a VL region that contains a CDR-L1 as set forth in SEQ ID NO:230, a CDR-L2 as set forth in SEQ ID NO:231, and a CDR-L3 as set forth in SEQ ID NO:232. In some of any of the provided embodiments, one of the first antigen binding domain or the second antigen binding domain contain a VH region and a VL region that contain the amino acid sequences set forth in SEQ ID NOS:197 and 198, respectively. In any of the provided embodiments, the first antigen binding domain or the second antigen binding domain contains the amino acid sequence set forth in SEQ ID NO:241 or a sequence of amino acids that exhibits at least at or about 90%, at least about or about 91%, at least at or about 92%, at least at or about 93%, at least at or about 94%, at least at or about 95%, at least at or about 96%, at least at or about 97%, at least at or about 98%, at least at or about 99% sequence identity to SEQ ID NO:241.

In any of the provided embodiments, one of the first antigen binding domain or the second antigen binding domain contains a VH region contains a CDR-H1 as set forth in SEQ ID NO:125, a CDR-H2 as set forth in SEQ ID NO:126, and a CDR-H3 as set forth in SEQ ID NO:127 and a VL region that contains a CDR-L1 as set forth in SEQ ID NO:130, a CDR-L2 as set forth in SEQ ID NO:131, and a CDR-L3 as set forth in SEQ ID NO:132. In any of the provided embodiments, one of the first antigen binding domain or the second antigen binding domain contain a VH region and VL region that contain the amino acid sequences set forth in SEQ ID NOS:27 and 28, respectively. In any of the provided embodiments, one of the first antigen binding domain or the second antigen binding domain contain the amino acid sequence set forth in SEQ ID NO:8 or a sequence of amino acids that exhibits at least at or about 90%, at least about or about 91%, at least at or about 92%, at least at or about 93%, at least at or about 94%, at least at or about 95%, at least at or about 96%, at least at or about 97%, at least at or about 98%, at least at or about 99% sequence identity to SEQ ID NO:8.

In any of the provided embodiments, one of the first antigen binding domain or the second antigen binding domain contains a VH region that contains a CDR-H1 as set forth in SEQ ID NO:209, a CDR-H2 as set forth in SEQ ID NO:210, and a CDR-H3 as set forth in SEQ ID NO:211 and a VL region that contains a CDR-L1 as set forth in SEQ ID NO:230, a CDR-L2 as set forth in SEQ ID NO:231, and a CDR-L3 as set forth in SEQ ID NO:232; and the other of the first antigen binding domain or the second antigen binding domain contains a CDR-H1 as set forth in SEQ ID NO:125, a CDR-H2 as set forth in SEQ ID NO:126, and a CDR-H3 as set forth in SEQ ID NO:127 and a VL region that contains a CDR-L1 as set forth in SEQ ID NO:130, a CDR-L2 as set forth in SEQ ID NO:131, and a CDR-L3 as set forth in SEQ ID NO:132. In some of any of the provided embodiments, one of the first antigen binding domain or the second antigen binding domain contain a VH region and a VL region that contain the amino acid sequences set forth in SEQ ID NOS:197 and 198, respectively; and the other of the first antigen binding domain or the second antigen binding domain contains a VH region and VL region that contain the amino acid sequences set forth in SEQ ID NOS:27 and 28, respectively. In some of any of the provided embodiments, one of the first or second antigen binding domain contains the amino acid sequence set forth in SEQ ID NO:241 and the other of the first or second antigen binding domain contains the amino acid sequence set forth in SEQ ID NO:8.

In some of any of the provided embodiments, one of the first or second antigen binding domain is encoded by the nucleotide sequence set forth in SEQ ID NO:310. In some of any of the provided embodiments, one of the first or second antigen binding domain is encoded by a nucleotide sequence set forth in SEQ ID NO:264 or SEQ ID NO: 311. In some of any of the provided embodiments, the first or second antigen binding domain is encoded by the nucleotide sequence set forth in SEQ ID NO:310, and the other of the first or second antigen binding domain is encoded by the nucleotide sequence set forth in SEQ ID NO:311

In some of any of the provided embodiments, (b) is or contains a spacer. In some of any of the provided embodiments, (b) contains a portion of an immunoglobulin. In some of any of the provided embodiments, (b) contains a sequence of a hinge region, a CH2 and CH3 region. In some of any of the provided embodiments, the hinge region contains all or a portion of an IgG4 hinge region and/or an IgG2 hinge region, wherein the IgG4 hinge region is optionally a human IgG4 hinge region and the IgG2 hinge region is optionally a human IgG2 hinge region; the C_{H}2 region contains all or a portion of an IgG4 C_{H}2 and/or an IgG2 C_{H}2, wherein the IgG4 C_{H}2 is optionally a human IgG4 C_{H}2 and the IgG2 C_{H}2 is optionally a human IgG2 C_{H}2; and/or the C_{H}3 region contains all or a portion of an IgG4 C_{H}3 and/or an IgG2 C_{H}3, wherein the IgG4 C_{H}3 is optionally a human IgG4 C_{H}3 and the IgG2 C_{H}3 is optionally a human IgG2 C_{H}3. In some of any of the provided embodiments, the hinge region, CH2 and CH3 contains all or a portion of a hinge, all or a portion of a C_{H}2 and all or a portion of a C_{H}3 from human IgG4. In some of any of the provided embodiments, one or more of the hinge region, the C_{H}2 and the C_{H}3 is chimeric and contains a hinge, C_{H}2 and C_{H}3 from human IgG4 and human IgG2. In some of any of the provided embodiments, (b) contains an IgG4/2 chimeric hinge region or a modified IgG4 hinge region containing at least one amino acid replacement compared to a human IgG4 hinge; an IgG2/4 chimeric CH2 region; and an IgG4 CH3 region.

In some of any of the provided embodiments, (b) is or contains a spacer. In some of any of the provided embodiments (b) has a length from or from about 125 to 300 amino acids in length, 125 to 250 amino acids in length, 125 to 230 amino acids in length, 125 to 200 amino acids in length, 125 to 180 amino acids in length, 125 to 150 amino acids in length, 150 to 300 amino acids in length, 150 to 250 amino acids in length, 150 to 230 amino acids in length, 150 to 200 amino acids in length, 150 to 180 amino acids in length, 180 to 300 amino acids in length, 180 to 250 amino acids in length, 180 to 230 amino acids in length, 180 to 200 amino acids in length, 200 to 300 amino acids in length, 200 to 250 amino acids in length, 200 to 230 amino acids in length, 230 to 300 amino acids in length, 230 to 250 amino acids in length or 250 to 300 amino acids in length, optionally wherein the spacer is at or about 224, at or about 225, at or about 226, at or about 227, at or about 228 or at or about 229 amino acids in length. In some of any of the provided embodiments, (b) is or contains the amino acid sequence set forth in SEQ ID NO:17. In some of any of the provided embodiments, (b) in one of the first CAR or the second CAR is encoded by the nucleotide sequence set forth in SEQ ID NO:48 and (b) in the other of the first CAR or the second CAR is encoded by the nucleotide sequence set forth in SEQ ID NO:305.

In some of any of the provided embodiments, (c) is or contains a transmembrane domain. In some of any of the provided embodiments, (c) is or contains a transmembrane domain of CD4, CD28, or CD8, optionally a transmembrane domain from human CD4, human CD28 or human CD8. In some of any of the provided embodiments, (c) is or contains a human CD28 transmembrane domain. In some of any of the provided embodiments, (c) is or contains the amino acid sequence set forth in SEQ ID NO:18. In some of any of the provided embodiments, (c) in one of the first CAR or the second CAR is encoded by the nucleotide sequence set forth in SEQ ID NOS:56 and (c) in the other of the first CAR or second CAR is encoded by the nucleotide sequence set forth in SEQ ID NO: 307.

In some of any of the provided embodiments, (d) is or contains an intracellular signaling region containing an intracellular signaling domain and a costimulatory signaling region. In some of any of the provided embodiments, the intracellular signaling domain of (d) is capable of inducing a primary activation signal in a T cell, is a T cell receptor (TCR) component and/or contains an immunoreceptor tyrosine-based activation motif (ITAM). In some of any of the provided embodiments, the intracellular signaling domain of (d) is or contains a cytoplasmic signaling domain of a CD3-zeta (CD3ζ) chain or a functional variant or signaling portion thereof, optionally a human CD3 zeta chain.). In some of any of the provided embodiments, the intracellular signaling domain of (d) is or contains the amino acid sequence set forth in SEQ ID NO:20. In some of any of the provided embodiments, the intracellular signaling domain of (d) in one of the first CAR or the second CAR is encoded by the nucleotide sequence set forth in SEQ ID NO:58 and the intracellular signaling domain of (d) in the other of the first CAR or the second CAR is encoded by the nucleotide sequence set forth in SEQ ID NO:309.In some of any of the provided embodiments, (d) is or contains an intracellular signaling region containing an intracellular signaling domain and a costimulatory signaling region. In some of any of the provided embodiments, the costimulatory signaling region of (d) contains an intracellular signaling domain of a T cell costimulatory molecule or a signaling portion thereof. In some of any of the provided embodiments, the costimulatory signaling region of (d) contains an intracellular signaling domain of CD28, 4-1BB, or ICOS, or a signaling portion thereof, optionally of human CD28, human 4-1BB, or human ICOS. In some of any of the provided embodiments, the costimulatory signaling region of (d) contains an intracellular signaling domain of 4-1BB. In some of any of the provided embodiments, the costimulatory signaling region of (d) is or contains the amino acid sequence set forth in SEQ ID NO:19. In some of any of the provided embodiments, the costimulatory signaling region of (d) in one of the first CAR or the second CAR is encoded by the nucleotide sequence set forth in SEQ ID NOS:60 and the costimulatory signaling region of (d) in the other of the first CAR or the second CAR is encoded by the nucleotide sequence set forth in SEQ ID NO:308.

In some of any of the provided embodiments, (a) is or contains the first antigen binding domain or the second antigen binding domain, (b) is or contains a spacer, (c) is or contains a transmembrane domain, and (d) is or contains an intracellular signaling region containing an intracellular signaling domain and a costimulatory signaling region. In some of any of the provided embodiments, one of the first CAR or the second CAR contains (a) a first antigen binding domain that binds to GPRC5D, optionally wherein the first antigen binding domain is encoded by the nucleotide sequence set forth in SEQ ID NO:311, (b) a spacer encoded by the nucleotide set forth in SEQ ID NO:305, (c) a transmembrane domain encoded by the nucleotide sequence set forth in SEQ ID NO:307, and (d) an intracellular signaling region containing an intracellular signaling domain encoded by the nucleotide sequence set forth in SEQ ID NO:309 and a co-stimulatory signaling region encoded by the nucleotide sequence set forth in SEQ ID NO:308; the other of the first CAR or the second CAR contains (a) an antigen binding domain that binds to BCMA, optionally wherein the antigen binding domain is encoded by the nucleotide sequence set forth in SEQ ID NO:310, (b) a spacer encoded by the nucleotide set forth in SEQ ID NO:48, (c) a transmembrane domain encoded by the nucleotide sequence set forth in SEQ ID NO:56, and (d) an intracellular signaling region containing an intracellular signaling domain encoded by the nucleotide sequence set forth in SEQ ID NO:58 and a co-stimulatory signaling domain region encoded by the nucleotide sequence set forth in SEQ ID NO:60.

In some of any of the provided embodiments, the first nucleic acid sequence encoding the first CAR is located toward the 5′ end of the polynucleotide, relative to the second nucleic acid sequence encoding the first CAR. In some of any of the provided embodiments, the first CAR contains an antigen binding domain that binds to GPRC5D and the second CAR contains an antigen binding domain that binds to BCMA. In some of any of the provided embodiments, the first CAR contains an antigen binding domain that binds to BCMA and the second CAR contains an antigen binding domain that binds to GPRC5D.

Also provided herein are polynucleotides containing (i) a first nucleic acid sequence encoding a first chimeric antigen receptor (CAR), (ii) a second nucleic acid sequence encoding a second chimeric antigen receptor (CAR) and (iii) a nucleotide sequence encoding a multicistronic element, wherein the first nucleic acid encoding the first CAR and the second nucleic acid encoding the second CAR are separated by the multicistronic element; wherein the first CAR contains a first antigen binding domain that binds to GPRC5D, optionally wherein the first antigen binding domain is encoded by the nucleotide sequence set forth in SEQ ID NO:311; a spacer encoded by the nucleotide set forth in SEQ ID NO:305; a transmembrane domain encoded by the nucleotide sequence set forth in SEQ ID NO:307; and an intracellular signaling region containing an intracellular signaling domain encoded by the nucleotide sequence set forth in SEQ ID NO:309 and a co-stimulatory signaling region encoded by the nucleotide sequence set forth in SEQ ID NO:308; wherein the second CAR contains a second antigen binding domain that binds to BCMA optionally wherein the second antigen binding domain is encoded by the nucleotide sequence set forth in SEQ ID NO:310; a spacer encoded by the nucleotide set forth in SEQ ID NO:48; a transmembrane domain encoded by the nucleotide sequence set forth in SEQ ID NO:56; and an intracellular signaling region containing an intracellular signaling domain encoded by the nucleotide sequence set forth in SEQ ID NO:58 and a co-stimulatory signaling domain region encoded by the nucleotide sequence set forth in SEQ ID NO:60; and wherein the first nucleic acid sequence encoding the first CAR is located toward the 5′ end of the polynucleotide relative to the second nucleic acid sequence encoding the second CAR.

Also provided herein are polynucleotides containing (i) a first nucleic acid sequence encoding a first chimeric antigen receptor (CAR), (ii) a second nucleic acid sequence encoding a second chimeric antigen receptor (CAR), and (iii) a nucleotide sequence encoding a multicistronic element, wherein the first nucleic acid encoding the first CAR and the second nucleic acid encoding the second CAR are separated by the multicistronic element; wherein the first CAR contains a first antigen binding domain that binds to BCMA, optionally wherein the first antigen binding domain is encoded by the nucleotide sequence set forth in SEQ ID NO:310, a spacer encoded by the nucleotide set forth in SEQ ID NO:48, a transmembrane domain encoded by the nucleotide sequence set forth in SEQ ID NO:56, and an intracellular signaling region containing an intracellular signaling domain encoded by the nucleotide sequence set forth in SEQ ID NO:58 and a co-stimulatory signaling domain region encoded by the nucleotide sequence set forth in SEQ ID NO:60 wherein the second CAR contains a second antigen binding domain that binds to GPRC5D, optionally wherein the second antigen binding domain is encoded by the nucleotide sequence set forth in SEQ ID NO:311, a spacer encoded by the nucleotide set forth in SEQ ID NO:305, a transmembrane domain encoded by the nucleotide sequence set forth in SEQ ID NO:307, and an intracellular signaling region containing an intracellular signaling domain encoded by the nucleotide sequence set forth in SEQ ID NO:309 and a co-stimulatory signaling region encoded by the nucleotide sequence set forth in SEQ ID NO:308; wherein the first nucleic acid encoding the first CAR is located toward the 5′ end of the polynucleotide relative to the second nucleic acid sequence encoding the second CAR.

In some of any of the provided embodiments, the multicistronic element contains the amino acid sequence set forth in SEQ ID NO:37. In some of any of the provided embodiments, the multicistronic element is encoded by a nucleotide sequence set forth in SEQ ID NOS:44 or SEQ ID NO: 45. In some of any of the provided embodiments, the multicistronic element is encoded by a nucleotide sequence set forth in SEQ ID NO:44. In some of any of the provided embodiments, the multicistronic element is encoded by a nucleotide sequence set forth in SEQ ID NO:45. In some of any of the provided embodiments, the multicistronic element is encoded by a nucleotide sequence set forth in SEQ ID NO:319.

In some of any of the provided embodiments, the polynucleotide contains the nucleotide sequence set forth in SEQ ID NO:299. In some of any of the provided embodiments, the polynucleotide encodes sequence set forth in SEQ ID NO:298.

In some of any of the provided embodiments, the polynucleotide contains the nucleotide sequence set forth in SEQ ID NO:302. In some of any of the provided embodiments, the polynucleotide encodes the sequence set forth in SEQ ID NO:301.

In some of any of the provided embodiments, the polynucleotide contains the nucleotide sequence set forth in SEQ ID NO:315. In some of any of the provided embodiments, the polynucleotide contains the nucleotide sequence set forth in SEQ ID NO:316.

Also provided herein are polynucleotides, wherein a polynucleotide encodes a GPRC5D-binding domain, a BCMA-binding domain, and an intracellular signaling region containing an intracellular signaling domain of a 4-1BB. In some of any of the provided embodiments, the polynucleotide contains the nucleotide sequence set forth in SEQ ID NO:317.

Also provided are vectors containing any of the provided polynucleotides. In some of any of the provided embodiments, the vector is a viral vector. In some of any of the provided embodiments, the viral vector is a lentiviral vector or a retroviral vector.

Also provided are engineered cells containing any of the chimeric antigen receptors provided herein. In some of any of the provided embodiments, the engineered cell contains a chimeric antigen receptor provided herein and further contains a polynucleotide containing a sequence of nucleotides encoding a second chimeric antigen receptor.

Also provided are engineered cells containing any of the polynucleotides provided herein.

In some of any of the provided embodiments, the engineered cell is a lymphocyte. In some of any of the provided embodiments, the engineered cell is an NK cell or a T cell. In some of any of the provided embodiments, the engineered cell is a T cell and the T cell is a CD4+ or a CD8+ T cell.

In some of any of the provided embodiments, the engineered cell was engineered from a primary cell obtained from a subject.

In some of any of the provided embodiments, the engineered cell is among a plurality of the engineered cells, where less than or less than about 10%, 9%, 8%, 7%, 5%, 4%, 3%, 2% or 1% of the cells in the plurality contain a chimeric antigen receptor that exhibits tonic signaling and/or antigen-independent activity or signaling.

Also provided are compositions containing any of the chimeric receptors provided herein. In some of any of the provided embodiments, the composition contains CD4+ and CD8+ T cells and the ratio of CD4+ to CD8+ T cells is from or from about 1:3 to 3:1. In some embodiments, the ratio of CD4+ and CD8+ T cells in the composition is 1:2 to 2:1. In some embodiments, the ratio of CD4+ and CD8+ T cells in the composition is 1:1. In some of any of the provided embodiments, the composition further contains a pharmaceutically acceptable excipient. In some of any of the provided embodiments, the composition is sterile.

Also provided herein are methods of treatment, including administering any of the compositions provided herein containing any of the engineered cells provided herein or any of the compositions provided herein containing any of the chimeric antigen receptors provided herein to a subject having a disease or disorder. In some of any of the provided embodiments, the dose of cells contains contain between at or about 2.5×10⁷ CAR-expressing T cells and 1.2×10⁹ CAR-expressing T cells, between at or about 5.0×10⁷ CAR-expressing T cells and 4.5×10⁸ CAR-expressing T cells, between at or about 1.5×10⁸ CAR-expressing T cells and 3.0×10⁸ CAR-expressing T cells. In some of any embodiments, the dose of cells contains between at or about 1×10⁷ CAR-expressing T cells and at or about 2×10⁹ CAR-expressing T cells. In some of any embodiments, the dose of cells contains between at or about 2.5×10⁷ CAR-expressing T cells and at or about 1.2×10⁹ CAR-expressing T cells, between at or about 5.0×10⁷ CAR-expressing T cells and at or about 4.5×10⁸ CAR-expressing T cells, or between at or about 1.5×10⁸ CAR-expressing T cells and at or about 3.0×10⁸ CAR-expressing T cells. In some of any embodiments, the dose of cells contains at or about 1.5×10⁷, at or about 2.5×10⁷, at or about 5.0×10⁷, at or about 7.5×10⁷, at or about 1.5×10⁸, at or about 2.25×10⁸, at or about 3.0×10⁸, at or about 4.5×10⁸, at or about 6.0×10⁸, at or about 8.0×10⁸ or at or about 1.2×10⁹ CAR-expressing T cells. In some of any embodiments, the dose of cells contains at or about 5.0×10⁷, at or about 1.5×10⁸, at or about 3.0×10⁸ or at or about 4.5×10⁸ CAR-expressing T cells. In some of any embodiments, the dose of cells contains at or about 5.0×10⁷, at or about 1.5×10⁸, at or about 3.0×10⁸ or at or about 4.5×10⁸ CAR-expressing T cells. In some of any embodiments, the dose of the cells contains at or about 5.0×10⁷ CAR-expressing T cells.

Also provided herein are methods of treatment, including administering any of the compositions provided herein containing any of the engineered cells provided herein or any of the compositions provided herein containing any of the chimeric antigen receptors provided herein to a subject having a disease or disorder. In some of any of the provided embodiments, the dose of cells contains contain between at or about 1.0×10⁷ CAR-expressing T cells and 1.2×10⁹ CAR-expressing T cells, between at or about 1.5×10⁷ CAR-expressing T cells and 4.5×10⁸ CAR-expressing T cells, between at or about 2.0×10⁷ CAR-expressing T cells and 3.0×10⁸ CAR-expressing T cells.

Also provided here in are methods of treatment, that include: administering a composition containing a plurality of engineered cells containing a first chimeric antigen receptor and a second chimeric antigen receptor, wherein each is any chimeric antigen receptor provided herein or encoded by any of the polynucleotides provided herein, to a subject having a disease or disorder; and administering to the subject a composition containing a plurality of second engineered cells containing a second chimeric antigen receptor. In some of any of the provided embodiments, the dose of the plurality of first engineered cells and the dose of the plurality of second engineered cells independently contain between at or about 1.0×10⁷ CAR-expressing T cells and 1.5×10⁹ CAR-expressing T cells, between at or about 1.25×10⁷ CAR-expressing T cells and 0.6×10⁸ CAR-expressing T cells, between at or about 2.5×10⁷ CAR-expressing T cells and 2.25×10⁸ CAR-expressing T cells, between at or about 7.5×10⁷ CAR-expressing T cells and 1.5×10⁸ CAR-expressing T cells, between at or about 2.5×10⁷ CAR-expressing T cells and 1.2×10⁹ CAR-expressing T cells, between at or about 5.0×10⁷ CAR-expressing T cells and 4.5×10⁸ CAR-expressing T cells, between at or about 1.5×10⁸ CAR-expressing T cells and 3.0×10⁸ CAR-expressing T cells.

In some of any of the provided embodiments of the provided methods, the disease or disorder is associated with expression of G protein-coupled receptor class C group 5 member D (GPRC5D).

In some of any of the provided embodiments of the provided methods, the disease or disorder is further associated with expression of B cell maturation antigen (BCMA).

In some of any of the provided embodiments of the provided methods, the disease or disorder is a B cell-related disorder. In some of any of the provided embodiments of the provided methods, the disease or disorder associated with BCMA is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

In some of any of the provided embodiments of the provided methods, the disease or disorder is a cancer. In some of any of the provided embodiments of the provided methods, the cancer is a GPRC5D-expressing cancer. In some of any of the provided embodiments of the provided methods, the cancer is a plasma cell malignancy and the plasma cell malignancy is multiple myeloma (MM) or plasmacytoma. In some of any of the provided embodiments of the provided methods, the cancer is multiple myeloma (MM). In some of any of the provided embodiments of the provided methods, the cancer is a relapsed/refractory multiple myeloma.

In some of any of the provided embodiments of the provided methods, the subject is refractory to or has relapsed following administration of a BCMA-targeted therapy, optionally following administration of T cells comprising a CAR that specifically binds BCMA. In some of any of the provided embodiments of the provided methods, a subject is selected for treatment that is refractory to or has relapsed following administration of a BCMA-targeted therapy, optionally following administration T cells comprising a CAR that specifically binds BCMA. In some of any of the provided embodiments of the provided methods, prior to the administration of the dose of cells, the subject has previously received administration of a BCMA-targeted therapy for treating the disease or disorder. In some of any of the provided embodiments of the provided methods, prior to the administration of the first dose of cells and the second dose of cells, the subject has previously received administration of a BCMA-targeted therapy for treating the disease or disorder.

In some of any of the provided embodiments of the provided methods, the BCMA-targeted therapy comprises a composition comprising T cells comprising a CAR that specifically binds BCMA. In some of any of the provided embodiments of the provided methods, the subject is refractory to or has relapsed following administration of the BCMA-targeted therapy, optionally following administration of T cells comprising a CAR that specifically binds BCMA. In some of any of the provided embodiments of the provided methods, the subject comprises multiple myeloma cells exhibiting BCMA antigen or epitope loss, BCMA downregulation and/or BCMA-negative tumor cells following a previous administration.

### BRIEF DESCRIPTION OF THE DRAWINGS

FIG. 1A shows Cancer Cell Line Encyclopedia CD138 mRNA expression data (log₂ scale). Type of cancer, from left to right: upper aerodigestive (32); esophagus (25); prostate (7); multiple myeloma (30); bile duct (8); lung (131); pancreas (44); kidney (34); breast (58); colorectal (61); stomach (38); meningioma (3); liver (28); glioma (62); osteosarcoma (10); thyroid (12); endometrium (27); soft tissue (21); mesothelioma (11); ovary (51); chondrosarcoma (4); small cell lung (53); melanoma (61); neuroblastoma (17); medulloblastoma (4); Ewing sarcoma (12); Hodgkin lymphoma (12); DLBCL (18); other lymphoma (28); B-cell (15); CML (15); Burkitt lymphoma (11); T-cell (16); AML (34); other leukemia (1).

FIG. 1B shows mRNA expression of GPRC5D in malignant cell lines [n=1036; Cancer Cell Line Encyclopedia (CCLE)]. RMA, robust multi-array average; DLBCL, diffuse large B cell lymphoma; CML, chronic myeloid leukemia; ALL, acute lymphoblastic leukemia; AML, acute myeloid leukemia; NSC, non-small cell. Type of cancer, from left to right: multiple myeloma (30); other leukemia (1); DLBCL (18); CML (15); meningioma (3); other lymphoma (28); Burkitt lymphoma (11); Hodgkin lymphoma (12); T-cell (16); B-cell (15); bile duct (8); AML (34); pancreas (44); thyroid (12); colorectal (61); kidney (34); osteosarcoma (10); urinary tract (27); breast (58); neuroblastoma (17); non-small cell lung (131); Ewing sarcoma (12); prostate (7); melanoma (61); upper aerodigestive (32); endometrium (27); medulloblastoma (4); liver (28); ovary (51); stomach (38); glioma (62); small cell lung (53); mesothelioma (11); esophagus (25); other (150); chondrosarcoma (4).

FIG. 2A shows CD138 GTEx RNASeq expression data for various organs. Type of tissue, from left to right: cerebellum, cerebral hemisphere, anterior cingulate cortex, frontal cortex, cortex, amygdala, hippocampus, nucleus accumbens, caudate, putamen, sigmoid colon, tibial nerve, skeletal muscle, uterus, muscularis of esophagus, gastroesophagal junction of esophagus, hypothalamus, adipose, cervix, coronary artery, cervical spinal cord, substantia nigra, ovary, tibial artery, mammary tissue, fallopian tube, adipose, kidney, left ventricle, cervix, adrenal gland, bladder, whole blood, skin (sun exposed), skin (not sun exposed), aortic artery, tonsil, small intestine, pancrease, liver, atrial appendage, vagina, stomach, prostate, spleen, cord blood, thyroid, transverse colon, pituitary, mucosa of esophagus, testis, minor salivary gland, lung, bone marrow.

FIG. 2B shows mRNA expression of GPRC5D in normal tissues according to Gtex RNASeq data (Gtex 708 ENSG00000111291.4). Dashed line represents the level of GPRC5D expression in CD138-sorted primary MM cells (Blueprint RNAseq; n=9). FPKM, fragments per kilobase of transcript per million mapped reads. Type of tissue, from left to right: cerebellum, cerebral hemisphere, anterior cingulate cortex, frontal cortex, cortex, amygdala, hippocampus, nucleus accumbens, caudate, putamen, sigmoid colon, tibial nerve, skeletal muscle, uterus, muscularis of esophagus, gastroesophagal junction of esophagus, hypothalamus, adipose, cervix, coronary artery, cervical spinal cord, substantia nigra, ovary, tibial artery, mammary tissue, fallopian tube, adipose, kidney, left ventricle, cervix, adrenal gland, bladder, whole blood, skin (sun exposed), skin (not sun exposed), aortic artery, tonsil, small intestine, pancrease, liver, atrial appendage, vagina, stomach, prostate, spleen, cord blood, thyroid, transverse colon, pituitary, mucosa of esophagus, testis, minor salivary gland, lung, primary MM (bone marrow).

FIG. 2C shows GPRC5D mRNA expression by Blueprint RNAseq for primary human tissue cell types. FPKM, fragments per kilobase of transcript per million mapped reads.

FIG. 3A shows Kaplan-Meier curves for progression free survival (PFS) stratified by subjects with MM whose GPRC5D expression by RNA-seq was either above (>) or below (<) median GPRC5D expression. Significance was determined by log-rank test of equal hazards (p=0.0031; n=765).

FIG. 3B shows international staging system (ISS) scores for subjects with MM, stratified by GPRC5D expression level (n=369 above median, 374 below median).

FIGS. 3C-3H show the frequency of common cytogenetic abnormalities among MM subjects, stratified by GPRC5D expression level (n=287-291 above median, 280-282 below median).

FIG. 4A shows outlier boxplot quantification of GPRC5D protein on cell lines, following immunohistochemical detection. The outlier boxplots indicate median membrane optical density and interquartile range (IQR); whiskers are 1.5×IQR. Mean fluorescence intensity (MFI) of GPRC5D expression in K562 cells engineered to express the protein is given by the number following the K562-GPRC5D designated cell line.

FIG. 4B shows automated quantitative immunofluorescence in 83 bone marrow samples from MM patients. Each column represents an individual patient sample.

FIG. 4C shows the percent of patient samples in which greater than 50% of CD138+ cells expressed BCMA, GPRC5D, or BCMA or GPRC5D as determined by automated quantitative immunofluorescence in 83 bone marrow samples from MM patients.

FIG. 4D shows the correlation of BCMA and GPRC5D expression on CD138+ cells; R²=0.156.

FIG. 5 shows linear, conformational, and discontinuous epitope binding of a subset of GPRC5D-targeted scFvs assessed by ELISA-based technology.

FIGS. 6A and 6B show antigen-independent (tonic) signaling of CARs containing the indicated scFvs and spacers. Jurkat Nur77-RFP reporter cells were transduced with 1 of 42 CAR/GFP bicistronic constructs. 5×10⁵ viable GFP+Jurkat cells were plated and monitored for RFP expression 11 days after transduction in the absence of target antigen. Expression of both RFP and GFP indicated tonic signaling; expression of GFP alone indicated CAR transduced without tonic signaling.

FIG. 6C-6E depict antigen-dependent vs. antigen-independent signaling of candidate CARs with long (FIG. 6C), medium (FIG. 6D), and short (FIG. 6E) spacers, measured after culturing Jurkat Nur77-RFP reporter cells 2:1 with MM.1S cells (expressing endogenous GPRC5D) for 20 h. Percent CAR T cell signaling determined by: RFP+GFP+/total GFP+ cells. Data representative of 2 experiments.

FIG. 6F shows CAR-transduced cells indicated as GFP+ along the y-axis. RFP, a surrogate for Nur77 expression, is shown along the x-axis. Percentages shown are of transduced GFP+ cells only (top quadrants only) that are RFP+.

FIG. 7A depicts binding of HEK293 cells transiently expressing one of a library of human G-protein coupled receptors (GPCR) with cytoplasmic GFP to co-cultured HEK293 cells transiently expressing anti-GPRC5D scFv clone 203, a long spacer, and cytoplasmic mCherry 761 (both in suspension), quantified by automated flow cytometric analysis. Pre-specified threshold for significance (red line): Z-score 3; p<0.0027.

FIG. 7B shows binding of anti-GPRC5D scFv clone 203 mIgG2a Fc chimeric antibody to HEK293 cells expressing the indicated cell surface proteins. Shown is confirmation of binding to potential off-target proteins and non-specific binders identified in a microarray screen of >4400 transmembrane proteins. ZsGreen1, transfection control; Isotype, irrelevant scFv-mIgG2a Fc negative control; CTLA-4/CD86 interaction, positive control.

FIG. 7C shows results of evaluation of potential off-target proteins PCDH1A or FCGR2A to activate through the GPRC5D (203) CAR. Jurkat Nur77-RFP activation reporter cells expressing a bicistronic plasmid containing a GPRC5D (203) CAR and GFP were co-cultured with K562 cells expressing the indicated antigens, GPRC5D (positive control), or BCMA (negative control). Activation was determined as % RFP+GFP+/total GFP+ cells.

FIG. 7D shows that CRISPR-Cas9-mediated knockout of GPRC5D from a MM cell line abolished activation of GPRC5D (203) CAR-Jurkat Nur77 reporter cells, as assessed by measuring changes in RFP expression by flow cytometry.

FIG. 8A shows GPRC5D mRNA expression across MM cell lines and primary MM cells (boxed).

FIG. 8B shows results of GPRC5D (203)-expressing CAR T cell cytotoxicity against MM1.S, OPM2, and RPMI-8226 target cells after 24 h of co-culture, as indicated by percent lysis, normalized to donor matched, mock-transduced CAR T cells (technical triplicates in each of two donors; mean±SD).

FIG. 9A shows cell killing of OPM2-ffLuc MM cells induced by CAR T cells incorporating the indicated scFv after 24 h of co-culture, as indicated by ATP-dependent bioluminescence after addition of luciferin; normalized to tumor cell-alone control (pooled data from 2 experiments each performed in triplicate, mean±SEM; p<0.001).

FIGS. 9B and 9C depict flow cytometry analysis depicting killing of primary bone marrow mononuclear cells (BMMCs) from a patient with multiply relapsed MM after overnight co-culture with anti-GPRC5D CART cells at a 1:1 ratio CAR+ T cells:BMMCs. MM cells, CD138+/CD38hi; plots gated on viable, CD3− cells.

FIG. 9D depicts flow cytometry analysis of primary BMMCs from additional patients, plotted for CD138+/CD3−.

FIGS. 10A to 10C show cytokines produced by CAR T cells incorporating the indicated scFv after co-culture 1:1 with OPM2 MM cells, or alone, for 24 h, measured in the supernatant by multiplex luminex assay.

FIGS. 11A and 11B show proliferation and FIGS. 11C and 11D show activation of mock-transduced or GPRC5D (203)-expressing CAR T cells cultured alone, with B-ALL (Nalm6; GPRC5D−), or MM (OPM2; endogenous GPRC5D+) cells at a 1:1 ratio. T cells were stained with CellTrace Violet (CTV) before co-culture, and stained for CD4, CD8, and CD25 after 72 h. (A, B) Proliferation is indicated by dilution of CTV fluorescence. (C, D) Activation is indicated by increased CD25 fluorescence.

FIG. 12A depicts representative FACS analysis of CAR expression in the CAR T cells, measured using an antibody specific to the spacer.

FIG. 12B depicts survival of mice treated 14 days post-OPM2 injection with 3×10⁶ 4-1BB containing CAR T cells incorporating the indicated anti-GPRC5D scFv clones (n=8/arm).

FIG. 12C depicts mouse tumor volume and survival in an RPMI-8226 xenograft model from one of two experiments; median days of survival 29 vs. 50 (p<0.05; n=5/arm, representative of two experiments).

FIG. 12D depicts mouse tumor volume and CAR T cell expansion in an RPMI-8226 xenograft model, as monitored by flow cytometry of peripheral blood using an antibody to the spacer to detect the CAR (p<0.001; n=10/arm; both timepoints).

FIG. 13A depicts survival of mice treated at 21 days post-OPM2 injection with 3×10⁶ T cells gene modified to express a bicistronic construct encoding extGLuc and a CAR incorporating scFv CD19 (SJ25C1) or GPRC5D (203) and either a 4-1BB or CD28 co-stimulatory domain (n=5/arm).

FIGS. 13B, C, and D depict tumor burden (D-luciferin bioluminescence imaging [BLI] of OPM-ffLuc) of mice from FIG. 13A.

FIG. 13E shows results of CAR T cell homing (coelenterazine BLI of extGLuc CAR T cells) of mice from FIG. 13A performed on day 7 post-CAR T cell treatment.

FIGS. 14A and 14B show dose response of GPRC5D-targeted and BCMA-targeted CAR T cell therapy, administered 14 days post-OPM2 injection (n=8 mice/arm). In FIG. 14A, tumor burden as assessed by BLI of OPM-ffLuc is shown. In FIG. 14B, percent survival is shown (p-values shown are vs. mock transduced or irrelevantly targeted CAR T cells).

FIG. 15A-15C depict IFN-gamma (FIG. 15A), TNF-alpha (FIG. 15B), and IL-2 (FIG. 15C) levels following 20 hours co-culture of GPRC5D (203), anti-BCMA, or mock-processed T cells with twenty different normal primary human cell types or OPM2 cells (mean±SD).

FIG. 15D depicts results from screening of murine and cynomolgus cross-reactive scFv clones for tonic signaling. % RFP+ indicates activation after co-culture at an effector:target ratio of 1:1 (relative to GFP+ CAR-transduced cells).

FIGS. 16A-C shows body mass change (FIG. 16A), body temperature (FIG. 16B) or BLI of OPM2-ffLuc cells (FIG. 16C) following injection of mice with 3×10⁶ human T cells expressing a CAR containing a human/murine cross-reactive anti-GPRC5D scFv (clone 205).

FIG. 17A shows representative FACs analysis of CAR expression as measured using a truncated receptor surrogate marker in non-human primate (NHP) T cells transduced to express either the cynomolgus cross-reactive GPRC5D CAR or cynomolgus GPRC5D.

FIG. 17B shows target lysis and FIG. 17C shows IFNγ production by NHP T cells transduced to express either the cynomolgus cross-reactive GPRC5D CAR or mock T cells against autologous target antigen presenting cells (tAPCs) at various effector to target (E:T) ratios.

FIG. 17D shows target lysis and FIG. 17E shows IFNγ production by NHP T cells transduced to express either the cynomolgus cross-reactive GPRC5D CAR or mock T cells against target K562 or K562-GPRC5D cells at various effector to target (E:T) ratios.

FIG. 18A shows results of PCR for the DNA encoding the CAR as a measure of CAR T cell persistence in the peripheral blood and bone marrow at day 21 after infusion. CAR transduced NHP T cells were used as a positive control.

FIG. 18B-D show results of pathologic evaluation 1 to 21 days after injection of cynomolgus monkeys with cynomolgus T cells modified to express a CAR containing a human/cynomolgus cross-reactive anti-GPRC5D scFv clone 202. FIG. 18B depicts body temperature, FIG. 18C depicts body mass change, and FIG. 18D depicts body mass.

FIG. 19A depicts BLI images at days 7 and 15 and FIG. 19B depicts images at day 34 of mice injected on day 0 with 1×10⁶ mixed population of OPM2WT cells and OPM2^{BCMA-KO} (GFP/ffLuc+) cells and injected on days 8 and 16 with 3×10⁶ of the indicated CAR T-cells. n=5 mice/arm, representative of 2 experiments.

FIGS. 20A and 20B show OPM2 cells in the bone marrow of mice injected with a mixed population OPM2 cells and CAR T-cells as described in FIGS. 19A and 19B. Representative plots of 3 mice per arm. Live/dead gating performed but not shown (n=2 replicate experiments with comparable results).

FIG. 21A shows minimal tonic signaling through an exemplary anti-BCMA CAR.

FIG. 21B shows lysis of target cells by primary human T cells expressing the exemplary anti-BCMA CAR.

FIG. 21C shows IFN-gamma secretion by primary human T cells expressing the exemplary anti-BCMA CAR upon co-culture with target cells.

FIG. 22A shows a loss of GPRC5D expression or BCMA expression, as assessed by flow cytometry, in OPM2 cells with GPRC5D or BCMA knocked out, respectively.

FIG. 22B shows antigen-specific activation of exemplary anti-BCMA and anti-GPRC5D CARs.

FIG. 23 shows BCMA and GPRC5D gene expression levels in multiple myeloma cell lines.

FIG. 24 shows BCMA and GPRC5D protein expression levels in multiple myeloma and control cell lines.

FIGS. 25A and 25B show OPM2 tumor burden in mice injected with either OPM2 WT cells (FIG. 25A), OPM2 BCMA KO cells (FIG. 25B; top panel) or OPM2 GPRC5D KO cells (FIG. 25B; bottom panel). Mice were treated with cell compositions containing cells expressing an anti-BCMA CAR (BCMA) or an anti-GPRC5D CAR (GPRC5D), or containing a pool of cells generated to contain anti-BCMA CAR-expressing cells and anti-GPRC5D CAR-expressing cells at a 1:1 ratio (GPRC5D and BCMA pooled cells).

FIG. 26 shows percent survival of mice injected with OPM2 tumor cells and treated with three different doses of cells expressing an anti-GPRC5D CAR (GPRC5D) or an anti-BCMA CAR (BCMA), or a pool of anti-BCMA CAR-expressing cells and anti-GPRC5D CAR-expressing cells (GPRC5D and BCMA pooled cells).

FIG. 27 shows tumor volume in mice injected with RPMI8226 cells and treated with three different doses of cells expressing an anti-GPRC5D CAR (GPRC5D) or an anti-BCMA CAR (BCMA), or a pool of anti-BCMA CAR-expressing cells and anti-GPRC5D CAR-expressing cells (GPRC5D and BCMA pooled cells).

FIG. 28 shows percent survival of mice from FIG. 27.

FIG. 29 depicts anti-BCMA and anti-GPRC5D dual-targeting strategies. (i) and (ii) represent pools of anti-BCMA CAR-expressing cells and anti-GPRC5D CAR-expressing cells (GPRC5D and BCMA pooled cells). (iii) and (iv) represent bicistronic constructs, each containing an anti-BCMA CAR and an anti-GPRC5D CAR separated by a self-cleaving peptide. (v) represents a “single stalk” CAR approach, wherein an anti-BCMA scFv and an anti-GPRC5D scFv are in tandem, separated only by a linker.

FIG. 30 shows expression of the indicated construct on the surface of cells, following retroviral transduction of cells with the respective constructs from FIG. 29.

FIG. 31 shows the retroviral transduction efficiency of each of the constructs depicted in FIG. 29, as assess by flow cytometric analysis.

FIG. 32A depicts the cytotoxicity of T cells expressing the constructs depicted in FIG. 29 upon co-culture with a wild-type OPM2 multiple myeloma cell line, as indicated by the percentage of lysed tumor cells. CAR-expressing T cells and target cells were cultured at increasing E:T ratios.

FIG. 32B depicts the cytotoxicity of cells expressing the constructs depicted in FIG. 29 upon co-culture with a BCMA knockout OPM2 cell line, as indicated by the percentage of lysed tumor cells. CAR-expressing T cells and target cells were cultured at increasing E:T ratios.

FIG. 33A shows the ability of T cells expressing the indicated CAR constructs to secrete various cytokines when co-cultured with BCMA- and GPRC5D-expressing target cells for 24 hours.

FIG. 33B shows the ability of T cells expressing the indicated CAR constructs to secrete various cytokines when co-cultured with BCMA-expressing, GPRC5D-negative target cells for 24 hours.

FIG. 33C shows the ability of T cells expressing the indicated CAR constructs to secrete various cytokines when co-cultured with GPRC5D-expressing, BCMA-negative target cells for 24 hours.

FIG. 34A depicts the survival of mice injected with OPM2 wild-type cells, following treatment with T cells expressing the indicated CAR(s).

FIG. 34B depicts the survival of mice from FIG. 34A after a second injection with BCMA knockout OPM2 cells, following treatment with T cells expressing the indicated CAR(s).

FIGS. 35A-C depict tumor growth, as assessed via bioluminescence imaging, in mice 30 days (FIG. 35A) or 105 days (FIG. 35B) after an initial injection with BCMA knockout OPM2 cells (2×10⁶), or 36 days (FIG. 35C) following a second injection with BCMA knockout OPM2 cells (3×10⁶), following treatment with 3×10⁶ CAR-expressing T cells.

FIG. 36 shows the survival of mice treated with a lower dose (5×10⁵) of cells expressing the indicated CAR(s), following injection with 2×10⁶ wild-type OPM2 cells.

FIGS. 37A-C depict tumor burden in mice, as assessed via bioluminescence imaging, injected with wild-type OPM2 cells, following 0 days (FIG. 37A), 15 days (FIG. 37B), or 22 days (FIG. 37C) of treatment with cells expressing the indicated CAR(s).

FIG. 38 depicts tumor burden in mice injected with a mixed composition of wild-type and 5-10% BCMA knockout OPM2 cells, as assessed via bioluminescence imaging of wild-type OPM2 cells (left panel) and BCMA knockout OPM2 cells (right panel), following treatment with 5×10⁵ cells expressing the indicated CAR(s).

FIG. 39 shows the survival of mice injected with a mixed composition of wild-type and 5-10% BCMA knockout OPM2 cells, following treatment with 2.5×10⁵ cells expressing the indicated CAR(s).

FIGS. 40A-C depict tumor burden in mice, as assessed via bioluminescence imaging, injected with a mixed composition of wild-type and 5-10% BCMA knockout OPM2 cells, 0 days (FIG. 40A), 22 days (FIG. 40B), or 34 days (FIG. 40C) following treatment with 5×10⁵ cells expressing the indicated CAR(s).

FIGS. 41A and 41B show loss of expression of the trailing CAR (BCMA and GPRC5D, respectively) in non-codon diverged bicistronic constructs.

FIGS. 42A and 42B show the codon divergence of the bicistronic constructs rescues expression of the trailing CAR (BCMA and GPRC5D, respectively).

FIG. 43 shows stimulation of Jurkat Nur77-RFP reporter cells expressing the indicated CAR(s) following co-culture with target cells.

FIGS. 44A-C show the expression of IFN-gamma, IL-2, and TNF-alpha (respectively) by primary human T cells expressing the indicated CAR(s), upon co-culture with target cells.

FIG. 45 shows antigen-specific activation of Jurkat Nur77-RFP reporter cells transduced with the indicated CAR(s), upon co-culture with OPM2 WT cells, OPM2 BCMA KO cells, or OPM2 GPRC5D KO cells.

FIGS. 46A-C show the expression of IFN-gamma, IL-2, and TNF-alpha (respectively) by primary human T cells expressing the indicated CAR(s), when cultured with OPM2 WT cells, OPM2 BCMA KO cells, or OPM2 GPRC5D KO cells.

FIG. 47A shows tumor burden (as assessed by BLI) in mice injected with OPM2 WT cells and treated with cells expressing the indicated CAR(s). FIGS. 47B and 47C show tumor burden (as assessed by BLI) in mice injected with a combination of OPM2 WT and BCMA KO cells (FIG. 47B) or a combination of OPM2 WT and GPRC5D KO cells (FIG. 47C) and treated with cells expressing the indicated CAR(s).

FIG. 48 shows the percent survival of mice from FIGS. 47A-C.

### DETAILED DESCRIPTION

Provided herein are chimeric antigen receptors (CARs) targeting or directed to G Protein-Coupled Receptor Class C Group 5 Member D (GPRC5D) and GPRC5D-expressing cells and disease. Also provided are cells, such as T cells, engineered to express a provided anti-GPRC5D CAR and compositions containing such cells. It is observed that GPRC5D is expressed, e.g., heterogeneously expressed, in certain diseases and conditions such as malignancies, or on tissues or cells thereof, e.g., on malignant plasma cells such as from relapsed or newly diagnosed myeloma patients, for example, with little expression on normal tissues. Among the provided embodiments are approaches useful in the treatment of diseases and conditions and/or for targeting such cell types, including nucleic acid molecules that encode GPRC5D-binding receptors, including chimeric antigen receptors (CARs), and the encoded receptors such as the encoded CARs, and compositions and articles of manufacture comprising the same. The receptors generally can contain antibodies (including antigen-binding antibody fragments, such as heavy chain variable (V_{H}) regions, single domain antibody fragments and single chain fragments, including scFvs) specific for GPRC5D. Also provided are cells, such as engineered or recombinant cells expressing such GPRC5D-binding receptors, e.g., anti-GPRC5D CARs and/or containing nucleic acids encoding such receptors, and compositions and articles of manufacture and therapeutic doses containing such cells.

Adoptive T cell therapies, such as CAR-T cell therapies, have shown promise for treating multiple myeloma, with clinical efforts primarily focused on targeting the B cell maturation antigen (BCMA). However, although BCMA is expressed on many malignant plasma cells, expression levels, in some cases, can be heterogeneous. In some aspects, heterogeneity in target antigen expression can lead to variable or inconsistent response. In some aspects, it also has been observed that expression of BCMA on the cell surface varies over time due to gamma secretase-mediated shedding of the extracellular domain Similar to observations with CD19 and CD22 CAR antigens, it has been reported that BCMA antigen down-regulation occurs in multiple myeloma (MM) patients who relapsed after BCMA-targeted T cell therapy (Brudno et al. (2018) J. Clin. Oncol., JCO2018778084; Cohen et al. (2017) Blood 130:505). Also, in some contexts, recombinant receptors can exhibit antigen-independent activity or signaling (also known as “tonic signaling”), which could lead to undesirable effects, such as due to increased differentiation and/or exhaustion of T cells that express the recombinant receptor. In some aspects, such activities may limit the T cell's activity, effect or potency. In some cases, during engineering and ex vivo expansion of the cells for recombinant receptor expression, the cells may exhibit phenotypes indicative of exhaustion, due to tonic signaling through the recombinant receptor. In some cases, alternative or additional MM-targeted T cell therapy approaches are needed.

The provided embodiments relate to GPRC5D as a CAR T cell target for multiple myeloma. GPRC5D (Uniprot Acc. No. Q9NZD1, e.g. set forth in SEQ ID NO:49) is a G protein coupled receptor class C, group 5 member D that belongs to the RAIG (retinoic acid-inducible gene-1) family. It is a seven transmembrane helix 39 kDa G-protein coupled receptor with two reported isoforms, with the isoform differences occurring in the intracellular C terminus of the protein. Results herein show that GPRC5D is expressed at high levels in multiple myeloma and, overall, it is expressed at low levels in most normal tissues.

The observations herein demonstrate protein expression of GPRC5D on multiple myeloma cells, supporting it as a feasible CAR T cell target for treating MM, including based on evaluation of potential on target/off tumor toxicity. Furthermore, among provided chimeric antigen receptors are chimeric receptors that display low tonic signaling, thereby minimizing possibility of antigen-independent (tonic) signaling. In particular, anti-GPRC5D CARs provided herein include CARs with high antigen-dependent activation and minimal tonic signaling. In particular, it is found that certain constructs, including those with a particular orientation of the variable heavy (VH) and variable light (VL) chain in the extracellular portion of the antibody fragment of the CAR and/or that contain a spacer of a certain length, exhibit advantageous properties including high antigen-dependent activation and low tonic signaling compared to alternative anti-GPRC5D CAR formats, such as those with shorter spacers.

In some embodiments, the spacer generally is a sequence of amino acids located between, such that connects, the extracellular antigen-binding domain and the transmembrane domain of the CAR. In particular embodiments of anti-GPRC5D CARs the spacer is a portion of an immunoglobulin, e.g. from IgG4 or IgG2, such as a portion containing, a hinge domain, a CH2 domain and a CH3 domain. Among such spacers are portions of human immunoglobulins or modified forms thereof, including those that have a length of greater than 125 amino acids in lengths, such as greater than 150 amino acids, greater than 180 amino acids, greater than 200 amino acids or greater than 200 amino acids in length. In some embodiments, an immunoglobulin spacer is a hybrid or chimeric spacers and/or is modified, such as to reduce or prevent glycosylation. In some embodiments, a provided anti-GPRC5D CAR includes an IgG4/IgG2 hinge-IgG4/IgG2 CH2-IgG4 CH3 immunoglobulin hybrid/modified spacer, such as set forth in SEQ ID NO:17.

In some embodiments, among CARs provided herein are those encoded by polynucleotides that are optimized, or contain certain features designed for optimization, such as for codon usage, to reduce RNA heterogeneity and/or to modify, e.g., increase or render more consistent among cell product lots, expression, such as surface expression, of the encoded receptor. In some embodiments, polynucleotides, encoding GPRC5D-binding cell surface proteins, are modified as compared to a reference polynucleotide, such as to remove cryptic or hidden splice sites, to reduce RNA heterogeneity. In some embodiments, polynucleotides, encoding GPRC5D-binding cell surface proteins, are codon optimized, such as for expression in a mammalian, e.g., human, cell, such as in a human T cell. In some aspects, the modified polynucleotides result in in improved, e.g., increased or more uniform or more consistent level of, expression, e.g., surface expression, when expressed in a cell. Such polynucleotides can be utilized in constructs for generation of engineered cells that express the encoded GPRC5D-binding cell surface protein. Thus, also provided are cells expressing the recombinant receptors encoded by the polynucleotides provided herein and uses thereof in adoptive cell therapy, such as treatment of diseases and disorders associated with GPRC5D expression, e.g., multiple myeloma.

Provided are monotherapy approaches utilizing anti-GPRC5D CAR expressed on autologous primary T cells for use as a therapeutic agent against multiple myeloma plasma cells. In some embodiments, a monotherapy approach may be desirable in subjects known or suspected or selected as having low or no BCMA-expressing MM plasma cells, and/or that have relapsed following remission, are refractory to, have failed treatment with or are intolerant to treatment with an anti-BCMA CAR.

Also provided herein are multi-targeting strategies that targets a first antigen and a second antigen associated with a particular disease or condition, such as multiple myeloma. In some embodiments, multiple recombinant receptors specifically bind or target different antigens are encoded by the same polynucleotide constructs, or included in the same cells, compositions, and methods provided herein. In some embodiments, the plurality of antigens, e.g., the first antigen and the second antigen, are expressed or suspected of being expressed on the cell, tissue, or disease or condition being targeted, such as on the cancer cell. In some aspects, the cell, tissue, disease or condition is multiple myeloma or a multiple myeloma cell.

For example, also provided herein is a dual therapy targeting approach of anti-GPRC5D CAR-expressing cells in combination with anti-BMCA CAR-expressing cells for use as a therapeutic agent against MM plasma cells. In some aspects, a dual targeting approach may be advantageous to overcome limitations associated with heterogeneous expression of BCMA and/or GPRC5D on MM plasma cells. It is observed that GPRC5D and BCMA are expressed, e.g., heterogeneously expressed, in certain diseases and conditions such as malignancies, or on tissues or cells thereof, e.g., on malignant plasma cells such as from relapsed or newly diagnosed myeloma patients, for example, with little expression on normal tissues. Due to the roles of GPRC5D and BCMA in various diseases and conditions, including cancer, both GPRC5D and BCMA are therapeutic targets.

In some cases, simultaneously targeting both antigens as provided herein may improve the depth and durability of responses across patients, in addition to minimizing relapse due to antigen escape. A mechanism of resistance to CAR T-cell therapies, as evidenced by data from CAR T-cell trials in B-cell malignancies, may be the loss or downregulation (“escape”) of the target antigen. (Robbie G. Majzner and Crystal L. Mackall, Cancer Discov Aug. 22, 2018; DOI 10.1158/2159-8290.CD-18-0442). Such a combination or dual targeting strategy may achieve synergistic or improved tumor responses based on targeting two antigens compared to monotherapy approaches involving only single antigen targeting. Indeed, studies herein demonstrate that BCMA and GPRC5D expression are independent of each other. A dual targeting approach may be advantageous to overcome problems due to potential for antigen loss and/or to maximize antigen targeting in MM. The observations herein demonstrate protein expression of GPRC5D, BCMA, or both, on multiple myeloma cells, supporting both antigens as feasible CAR T cell targets for treating MM, including based on evaluation of potential on target/off tumor toxicity.

Among the provided embodiments are approaches useful in the treatment of diseases and conditions and/or for targeting such cell types, including nucleic acid molecules that encode GPRC5D-binding receptors and BCMA-binding receptors, including chimeric antigen receptors (CARs), and the encoded receptors such as the encoded CARs, and compositions and articles of manufacture comprising the same. The receptors generally can contain antibodies (including antigen-binding antibody fragments, such as heavy chain variable (V_{H}) regions, single domain antibody fragments and single chain fragments, including single chain variable fragments (scFvs)) specific for GPRC5D or BCMA. Also provided are cells, such as engineered or recombinant cells expressing such GPRC5D-binding receptors, e.g., anti-GPRC5D CARs, and BCMA-binding receptors, e.g. anti-BCMA CARs, and/or containing nucleic acids encoding such receptors, and compositions and articles of manufacture and therapeutic doses containing such cells. Among provided embodiments are polynucleotides that are bicistronic for expression of multiple CARs, such as an anti-GPRCD CAR and an anti-BCMA CAR. The observations herein demonstrate that expression of multiple CARs, e.g. an anti-GPRC5D CAR and an anti-BCMA CAR, in a cell can be improved by codon diverging a polynucleotide sequence encoding one or more of the CARs. It is found that codon divergence of a polynucleotide construct encoding two CARs improves expression of a nucleotide sequence encoding a CAR that is 3′prime (or C-terminal) relative to nucleotide sequence encoding the other CAR.

Additionally, it is found that provided CARs containing a spacer of a certain length, exhibit advantageous properties including high antigen-dependent activation and low tonic signaling compared to alternative anti-GPRC5D or anti-BCMA CAR formats, such as those with shorter spacers. In some embodiments, the spacer component of a CAR generally is a sequence of amino acids located between, such that connects, the extracellular antigen-binding domain and the transmembrane domain of the CAR. In particular embodiments of an anti-GPRC5D or an anti-BCMA CAR, the spacer is a portion of an immunoglobulin, e.g. from IgG4 or IgG2, such as a portion containing, a hinge domain, a CH2 domain and a CH3 domain Among such spacers are portions of human immunoglobulins or modified forms thereof, including those that have a length of greater than 125 amino acids in lengths, such as greater than 150 amino acids, greater than 180 amino acids, greater than 200 amino acids or greater than 200 amino acids in length. In some embodiments, an immunoglobulin spacer is a hybrid or chimeric spacers and/or is modified, such as to reduce or prevent glycosylation. In some embodiments, a provided anti-GPRC5D or anti-BCMA CAR includes an IgG4/IgG2 hinge-IgG4/IgG2 CH2-IgG4 CH3 immunoglobulin hybrid/modified spacer, such as set forth in SEQ ID NO:17. In particular embodiments, the polynucleotide encoding the CAR contains a spacer region that has been modified to eliminate splice sites, such as cryptic splice and/or acceptor sites. Exemplary nucleotides encoding the spacer are described. In some embodiments, the coding sequence for the spacer comprises the nucleic acid sequence set forth in SEQ ID NO: 48 (also set forth in SEQ ID NO: 74). In some embodiments, the provided CARs exhibit reduced RNA heterogeneity when expressed in cells (e.g. T cells). In some embodiments, the provided polynucleotides encoding the CARs also can be codon optimized to further improve expression.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. GPRC5D-Binding Receptors and Encoding Polynucleotides

Provided in some aspects are GPRC5D-binding agents, such as recombinant receptors or chimeric antigen receptors that bind GPRC5D molecules and polynucleotides encoding GPRC5D binding cell surface proteins, such as recombinant receptors (e.g., CARs), and cells expressing such receptors. The GPRC5D-binding cell surface proteins generally contain antibodies (e.g., antigen-binding antibody fragments), and/or other binding peptides that specifically bind to GPRC5D, such as to GPRC5D proteins, such as human GPRC5D protein. In some aspects, the agents bind to an extracellular portion of GPRC5D.

Among the provided polynucleotides are those that encode recombinant receptors, such as antigen receptors, that specifically bind GPRC5D. In some aspects, the encoded receptors, such as those containing GPRC5D-binding polypeptides, and compositions and articles of manufacture and uses of the same, also are provided.

Among the GPRC5D-binding polypeptides are antibodies, such as single-chain antibodies (e.g., antigen binding antibody fragments), or portions thereof. In some examples, the recombinant receptors are chimeric antigen receptors, such as those containing anti-GPRC5D antibodies or antigen-binding fragments thereof. The provided polynucleotides can be incorporated into constructs, such as deoxyribonucleic acid (DNA) or RNA constructs, such as those that can be introduced into cells for expression of the encoded recombinant GPRC5D-binding receptors.

1. Components of Encoded Recombinant GPRC5D-Binding Receptors

The provided GPRC5D-binding receptors generally contain an extracellular binding molecule and an intracellular signaling domain Among the provided receptors are polypeptides containing antibodies, such as recombinant cell surface receptors containing anti-GPRC5D antibodies. Such receptors include chimeric antigen receptors that contain such antibodies.

Among the provided recombinant receptors are antigen receptors that include a GPRC5D-binding fragment. The recombinant receptors include antigen receptors that specifically bind to GPRC5D, such as antigen receptors containing the anti-GPRC5D antibodies, e.g., GPRC5D antigen-binding fragments. Among the antigen receptors are functional non-TCR antigen receptors, such as chimeric antigen receptors (CARs). Also provided are cells expressing the recombinant receptors and uses thereof in adoptive cell therapy, such as treatment of diseases and disorders associated with GPRC5D expression, e.g., multiple myeloma.

a. Extracellular Antigen-Binding Domain

Among the chimeric receptors are chimeric antigen receptors (CARs). The chimeric receptors, such as CARs, generally include an extracellular antigen binding domain that includes, is, or comprises an anti-GPRC5D antibody. Thus, the chimeric receptors, e.g., CARs, typically include in their extracellular portions one or more GPRC5D-binding molecules, such as one or more antigen-binding fragment, domain, or portion, or one or more antibody variable regions, and/or antibody molecules, such as those described herein.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM or Contact method or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{H} or V_{L} domains, respectively (see, e.g., Portolano et al., J. Immunol. 150:880-887 (1993); Clarkson et al., Nature 352:624-628 (1991)).

Among the antibodies included in the provided CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; heavy chain variable (V_{H}) regions, single-chain antibody molecules such as scFvs and single-domain antibodies comprising only the V_{H} region; and multispecific antibodies formed from antibody fragments. In some embodiments, the antigen-binding domain in the provided CARs is or comprises an antibody fragment comprising a variable heavy chain (V_{H}) and a variable light chain (V_{L}) region. In particular embodiments, the antibodies are single-chain antibody fragments comprising a heavy chain variable (V_{H}) region and/or a light chain variable (V_{L}) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the anti-GPRC5D antibodies included in the provided CARs are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the antibodies included in the provided CARs are those that are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

In some embodiments, the CAR includes a GPRC5D-binding portion or portions of the antibody molecule, such as a heavy chain variable (V_{H}) region and/or light chain variable (V_{L}) region of the antibody, e.g., an scFv antibody fragment. In some embodiments, the provided GPRC5D-binding CARs contain an antibody, such as an anti-GPRC5D antibody, or an antigen-binding fragment thereof that confers the GPRC5D-binding properties of the provided CAR. In some embodiments, the antibody or antigen-binding domain can be any anti-GPRC5D antibody described or derived from any anti-GPRC5D antibody described (see, e.g., WO 2016/090312, WO 2016/090329, WO 2018/017786). Any of such anti-GPRC5D antibodies or antigen-binding fragments can be used in the provided CARs. In some embodiments, the anti-GPRC5D CAR contains an antigen-binding domain that is an scFv containing a variable heavy (V_{H}) and/or a variable light (V_{L}) region derived from an antibody described in WO 2016/090312, WO 2016/090329, or WO 2018/017786.

In some embodiments, the antibody, e.g., the anti-GPRC5D antibody, or antigen-binding fragment, contains a heavy and/or light chain variable (V_{H} or V_{L}) region sequence as described, or a sufficient antigen-binding portion thereof. In some embodiments, the anti-GPRC5D antibody, e.g., antigen-binding fragment, contains a V_{H} region sequence or sufficient antigen-binding portion thereof that contains a CDR-H1, CDR-H2 and/or CDR-H3 as described. In some embodiments, the anti-GPRC5D antibody, e.g., antigen-binding fragment, contains a V_{L} region sequence or sufficient antigen-binding portion that contains a CDR-L1, CDR-L2 and/or CDR-L3 as described. In some embodiments, the anti-GPRC5D antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence.

In some embodiments, the antibody or antibody fragment, in the provided CAR, has a V_{H} region of any of the antibodies or antibody binding fragments described in any of WO 2016/090312, WO 2016/090329, and WO 2018/017786.

In some embodiments, the CAR contains an antibody or antigen-binding fragment thereof, that has a heavy chain variable (V_{H}) region having the amino acid sequence selected from any one of SEQ ID NOs: 21, 23, 25, 27, 29, 31, or 33, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid selected from any one of SEQ ID NOs: 21, 23, 25, 27, 29, 31, or 33, or contains a CDR-H1, CDR-H2, and/or CDR-H3 present in such a V_{H} sequence.

In some embodiments, the V_{H} region of an antibody or antigen-binding fragment thereof comprises a CDR-H1, CDR-H2, and/or CDR-H3 according to Kabat numbering. In some embodiments, the V_{H} region of an antibody or antigen-binding fragment thereof comprises a CDR-H1, CDR-H2, and/or CDR-H3 according to Chothia numbering. In some embodiments, the V_{H} region of an antibody or antigen-binding fragment thereof comprises a CDR-H1, CDR-H2, and/or CDR-H3 according to AbM numbering.

In some embodiments, the CAR contains an antibody or antigen-binding fragment thereof, that has a variable heavy chain (V_{H}) region comprising a CDR-H1 comprising the amino acid sequence selected from SEQ ID NOs: 75, 78, 80, 82, 90, 93, 95, 97, 105, 108, 110, 112, 120, 123, 125, 127, 135, 138, 140, 142, 152, 162, 165, 167, and 169; (b) a CDR-H2 comprising the amino acid sequence selected from SEQ ID NOs: 76, 79, 81, 83, 91, 94, 96, 98, 106, 109, 111, 113, 121, 124, 126, 128, 136, 139, 141, 143, 150, 153, 154, 155, 163, 166, 168, and 170; and (c) a CDR-H3 comprising the amino acid sequence selected from SEQ ID NOs: 77, 84, 92, 99, 107, 114, 122, 129, 137, 144, 151, 156, 164, and 171.

In some embodiments, the antibody or antigen-binding fragment thereof comprises a V_{H} region comprising a CDR-H1, CDR-H2, and CDR-H3 comprising the amino acid sequence of SEQ ID NOs:75, 76 and 77, respectively; SEQ ID NOs:78, 79 and 77, respectively; SEQ ID NOs:80, 81 and 77, respectively; SEQ ID NOs:82, 83 and 84, respectively; SEQ ID NOs:90, 91 and 92, respectively; SEQ ID NOs:93, 94 and 92, respectively; SEQ ID NOs:95, 96 and 92, respectively; SEQ ID NOs:97, 98 and 99, respectively; SEQ ID NOs:105, 106 and 107, respectively; SEQ ID NOs:108, 109 and 107, respectively; SEQ ID NOs:110, 111 and 107, respectively; SEQ ID NOs:112, 113 and 114, respectively; SEQ ID NOs:120, 121 and 122, respectively; SEQ ID NOs:123, 124 and 122, respectively; SEQ ID NOs:125, 126 and 122, respectively; SEQ ID NOs:127, 128 and 129, respectively; SEQ ID NOs:135, 136 and 137, respectively; SEQ ID NOs:138, 139 and 137, respectively; SEQ ID NOs:140, 141 and 137, respectively; SEQ ID NOs:142, 143 and 144, respectively; SEQ ID NOs:135, 150 and 151, respectively; SEQ ID NOs:152, 153 and 151, respectively; SEQ ID NOs:140, 154 and 151, respectively; SEQ ID NOs:142, 155 and 156, respectively; SEQ ID NOs:162, 163 and 164, respectively; SEQ ID NOs:165, 166 and 164, respectively; SEQ ID NOs:167, 168 and 164, respectively; SEQ ID NOs:169, 170 and 171, respectively.

In some embodiments, the antibody or antigen-binding fragment thereof comprises a V_{H} region comprising the amino acid sequence of SEQ ID NOs:75, 76 and 77, respectively; SEQ ID NOs:78, 79 and 77, respectively; SEQ ID NOs:80, 81 and 77, respectively; SEQ ID NOs:82, 83 and 84, respectively; SEQ ID NOs:90, 91 and 92, respectively; SEQ ID NOs:93, 94 and 92, respectively; SEQ ID NOs:95, 96 and 92, respectively; SEQ ID NOs:97, 98 and 99, respectively; SEQ ID NOs:105, 106 and 107, respectively; SEQ ID NOs:108, 109 and 107, respectively; SEQ ID NOs:110, 111 and 107, respectively; SEQ ID NOs:112, 113 and 114, respectively; SEQ ID NOs:120, 121 and 122, respectively; SEQ ID NOs:123, 124 and 122, respectively; SEQ ID NOs:125, 126 and 122, respectively; SEQ ID NOs:127, 128 and 129, respectively; SEQ ID NOs:135, 136 and 137, respectively; SEQ ID NOs:138, 139 and 137, respectively; SEQ ID NOs:140, 141 and 137, respectively; SEQ ID NOs:142, 143 and 144, respectively; SEQ ID NOs:135, 150 and 151, respectively; SEQ ID NOs:152, 153 and 151, respectively; SEQ ID NOs:140, 154 and 151, respectively; SEQ ID NOs:142, 155 and 156, respectively; SEQ ID NOs:162, 163 and 164, respectively; SEQ ID NOs:165, 166 and 164, respectively; SEQ ID NOs:167, 168 and 164, respectively; SEQ ID NOs:169, 170 and 171, respectively.

In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in any one of SEQ ID NOs: 21, 23, 25, 27, 29, 31, or 33.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in any one of SEQ ID NOs: 21, 23, 25, 27, 29, 31, or 33.

In some embodiments, the antibody or antigen-binding fragment thereof comprises a V_{H} region comprising the amino acid sequence set forth in any one of SEQ ID NOs: 21, 23, 25, 27, 29, 31, or 33.

In some embodiments, the antibody or antibody fragment, in the provided CAR (e.g. an anti-GPRC5D CAR), comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, a CAR provided herein, contains an antibody such as an anti-GPRC5D antibody, or antigen-binding fragment thereof that contains any of the above V_{H} region and contains a variable light chain region or a sufficient antigen binding portion thereof. For example, in some embodiments, the CAR contains an antibody or antigen-binding fragment thereof that contains a V_{H} region and a variable light chain (V_{L}) region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the antibody or antigen-binding fragment has a V_{L} region described in any of WO 2016/090312, WO 2016/090329, and WO 2018/017786.

In some embodiments, the CAR contains an antibody or antigen-binding fragment thereof, that has a light chain variable (V_{L}) region having the amino acid sequence selected from any one of SEQ ID NOs: 22, 24, 26, 28, 30, 32, 34, 63, 64, 65, 66, 67, 68, or 69, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid selected from any one of SEQ ID NOs: 22, 24, 26, 28, 30, 32, 34, 63, 64, 65, 66, 67, 68, or 69, or contains a CDR-L1, CDR-L2, and/or CDR-L3 present in such a V_{L} sequence. In some embodiments, the CAR contains an antibody or antigen-binding fragment thereof, that has a light chain variable (V_{L}) region having the amino acid sequence selected from any one of SEQ ID NOs: 22, 24, 26, 28, 30, 32, or 34, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid selected from any one of SEQ ID NOs: 22, 24, 26, 28, 30, 32, or 34, or contains a CDR-L1, CDR-L2, and/or CDR-L3 present in such a V_{L} sequence. In some embodiments, the CAR contains an antibody or antigen-binding fragment thereof, that has a light chain variable (V_{L}) region having the amino acid sequence selected from any one of SEQ ID NOs: 63, 64, 65, 66, 67, 68 or 69, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid selected from any one of SEQ ID NOs: 63, 64, 65, 66, 67, 68, or 69, or contains a CDR-L1, CDR-L2, and/or CDR-L3 present in such a V_{L} sequence.

In some embodiments, the V_{L} region of an antibody or antigen-binding fragment thereof comprises a CDR-L1, CDR-L2, and/or CDR-L3 according to Kabat numbering. In some embodiments, the V_{L} region of an antibody or antigen-binding fragment thereof comprises a CDR-L1, CDR-L2, and/or CDR-L3 according to Chothia numbering. In some embodiments, the V_{L} region of an antibody or antigen-binding fragment thereof comprises a CDR-L1, CDR-L2, and/or CDR-L3 according to AbM numbering.

In some embodiments, the CAR contains an antibody or antigen-binding fragment thereof, that has a variable light chain (V_{L}) region comprising a CDR-L1 comprising the amino acid sequence selected from SEQ ID NOs: 85, 88, 100, 103, 115, 118, 130, 133, 145, 148, 157, 160, 172, and 174; (b) a CDR-L2 comprising the amino acid sequence selected from SEQ ID NOs: 86, 89, 101, 104, 116, 119, 131, 134, 146, 149, 158, and 161; and (c) a CDR-L3 comprising the amino acid sequence selected from SEQ ID NOs: 87, 102, 117, 132, 147, 159, 173, and 175.

In some embodiments, the antibody or antigen-binding fragment thereof comprises a V_{L} region comprising a CDR-L1, CDR-L2, and CDR-L3 comprising the amino acid sequence of SEQ ID NOs:85, 86 and 87, respectively; SEQ ID NOs:88, 89 and 87, respectively; SEQ ID NOs:100, 101 and 102, respectively; SEQ ID NOs:103, 104 and 102, respectively; SEQ ID NOs:115, 116 and 117, respectively; SEQ ID NOs:118, 119 and 117, respectively; SEQ ID NOs:130, 131 and 132, respectively; SEQ ID NOs:133, 134 and 132, respectively; SEQ ID NOs:145, 146 and 147, respectively; SEQ ID NOs:148, 149 and 147, respectively; SEQ ID NOs:157, 158 and 159, respectively; SEQ ID NOs:160, 161 and 159, respectively; SEQ ID NOs:172, 86 and 173, respectively; SEQ ID NOs:174, 89 and 175, respectively; SEQ ID NOs:174, 89 and 297, respectively.

In some embodiments, the antibody or antigen-binding fragment thereof comprises a V_{L} region comprising the amino acid sequence of SEQ ID NOs:85, 86 and 87, respectively; SEQ ID NOs:88, 89 and 87, respectively; SEQ ID NOs:100, 101 and 102, respectively; SEQ ID NOs:103, 104 and 102, respectively; SEQ ID NOs:115, 116 and 117, respectively; SEQ ID NOs:118, 119 and 117, respectively; SEQ ID NOs:130, 131 and 132, respectively; SEQ ID NOs:133, 134 and 132, respectively; SEQ ID NOs:145, 146 and 147, respectively; SEQ ID NOs:148, 149 and 147, respectively; SEQ ID NOs:157, 158 and 159, respectively; SEQ ID NOs:160, 161 and 159, respectively; SEQ ID NOs:172, 86 and 173, respectively; SEQ ID NOs:174, 89 and 175, respectively; SEQ ID NOs:174, 89 and 297, respectively.

In some embodiments, the antibody or antigen-binding fragment thereof contains a CDR-L1, CDR-L2, and CDR-L3, respectively, contained within the V_{L} region amino acid sequence selected from any one of SEQ ID NOs: 22, 24, 26, 28, 30, 32, 34, 63, 64, 65, 66, 67, 68, or 69. In some embodiments, the antibody or antigen-binding fragment thereof contains a CDR-L1, CDR-L2, and CDR-L3, respectively, contained within the V_{L} region amino acid sequence selected from any one of SEQ ID NOs: 22, 24, 26, 28, 30, 32, or 34. In some embodiments, the antibody or antigen-binding fragment thereof contains a CDR-L1, CDR-L2, and CDR-L3, respectively, contained within the V_{L} region amino acid sequence selected from any one of SEQ ID NOs: 63, 64, 65, 66, 67, 68, or 69.

Among the CARs provided herein is a CAR in which the antibody, such as an anti-GPRC5D antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in any of SEQ ID NOs: 21, 23, 25, 27, 29, 31, or 33 and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in any one of SEQ ID NOs: 22, 24, 26, 28, 30, 32, 34, 63, 64, 65, 66, 67, 68, or 69.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence selected from any one of SEQ ID NOs: 21, 23, 25, 27, 29, 31, or 33; and comprises a CDR-L1, a CDR-L2, a CDR-L3, respectively, comprising the amino acid sequences of CDR-L1, CDR-L2, and CDR-L3, respectively contained within the V_{L} region amino acid sequence selected from any one of SEQ ID NOs: 22, 24, 26, 28, 30, 32, 34, 63, 64, 65, 66, 67, 68, or 69.

In some embodiments, the V_{H} region of the antibody or antigen-binding fragment thereof comprise the amino acid sequence of SEQ ID NOs: 21, 23, 25, 27, 29, 31, or 33 and the and V_{L} regions of the antibody or antigen-binding fragment comprises the amino acid sequence 22, 24, 26, 28, 30, 32, or 34. In some embodiments, the V_{H} and V_{L} regions of the antibody or antigen-binding fragment thereof comprise the amino acid sequences of SEQ ID NOs: 21 and 22, respectively; SEQ ID NOs: 23 and 24, respectively; SEQ ID NOs: 25 and 26, respectively; SEQ ID NOs: 27 and 28, respectively; SEQ ID NOs: 29 and 30, respectively; SEQ ID NOs: 31 and 32, respectively; or SEQ ID NOs: 33 and 34, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequences selected from: SEQ ID NOs: 21 and 22; SEQ ID NOs: 23 and 24; SEQ ID NOs: 25 and 26; SEQ ID NOs: 27 and 28; SEQ ID NOs: 29 and 30; SEQ ID NOs: 31 and 32; SEQ ID NOs: 33 and 34, respectively. In other examples, the V_{H} and V_{L} regions of the antibody or antigen-binding fragment thereof provided therein comprise the amino acid sequences selected from: SEQ ID NOs: 21 and 63; SEQ ID NOs: 23 and 64; SEQ ID NOs: 25 and 65; SEQ ID NOs: 27 and 66; SEQ ID NOs: 29 and 67; SEQ ID NOs: 31 and 68; SEQ ID NOs: 33 and 69, respectively.

In some embodiments, the antibody or antigen-binding fragment thereof, in the provided CAR, is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is an scFv comprising a heavy chain variable (V_{H}) region and a light chain variable (V_{L}) region. In some embodiments, the single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided CARs contain anti-GPRC5D antibodies that include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 50) or GGGS (3GS; SEQ ID NO: 51), such as between 2, 3, 4, and 5 repeats of such a sequence. Exemplary linkers include those having or consisting of a sequence set forth in SEQ ID NO: 52 (GGGGSGGGGSGGGGS). Exemplary linkers further include those having or consisting of the sequence set forth in SEQ ID NO: 53 (GSTSGSGKPGSGEGSTKG). Exemplary linkers further include those having or consisting of the sequence set forth in SEQ ID NO: 54 (SRGGGGSGGGGSGGGGSLEMA). An exemplary linker includes those having or consisting of the sequence set forth in SEQ ID NO: 47 (GSRGGGGSGGGGSGGGGSLEMA).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 51) or GGGGS (SEQ ID NO: 50), such as the linker set forth in SEQ ID NO: 47, 52 or 54.

In some embodiments, the V_{H} region may be amino terminal to the V_{L} region. In some embodiments, the V_{H} region may be carboxy terminal to the V_{L} region. In particular embodiments, the fragment, e.g., scFv, may include a V_{H} region or portion thereof, followed by the linker, followed by a V_{L} region or portion thereof. In other embodiments, the fragment, e.g., the scFv, may include the V_{L} region or portion thereof, followed by the linker, followed by the V_{H} region or portion thereof.

In some aspects, an scFv provided herein comprises the amino acid sequence selected from any one of SEQ ID NOs: 1-14, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from any one of SEQ ID NOs: 1-14.

Among a provided anti-GPRC5D CAR is a CAR in which the antibody or antigen-binding fragment contains a V_{H} region comprising the sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and contains a V_{L} region comprising the sequence set forth in SEQ ID NO:22 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:22. In some embodiments, the provided CAR is a CAR in which the antibody or antigen-binding fragment contains a V_{H} region comprising the sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and contains a V_{L} region comprising the sequence set forth in SEQ ID NO:63 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:63. In some embodiments, the antibody or antigen-binding fragment of the provided CAR contains a V_{H} region that has a CDRH1, a CDRH2 and a CDRH3 comprising the amino acid sequence of SEQ ID NOS: 75, 76 and 77, respectively and a V_{L} region that has a CDRL1, a CDRL2 and a CDRL3 comprising the amino acid sequence of SEQ ID NOS: 85, 86, and 87, respectively. In some embodiments, the antibody or antigen-binding fragment of the provided CAR contains a V_{H} region that has a CDRH1, a CDRH2 and a CDRH3 comprising the amino acid sequence of SEQ ID NOS: 78, 79 and 77, respectively and a V_{L} region that contains a CDRL1, a CDRL2 and a CDRL3 comprising the amino acid sequence of SEQ ID NOS: 85, 86, and 87, respectively. In some embodiments, the antibody or antigen-binding fragment of the provided CAR contains a V_{H} region that has a CDRH1, a CDRH2 and a CDRH3 comprising the amino acid sequence of SEQ ID NOS: 80, 81 and 77, respectively and a V_{L} region that contains a CDRL1, a CDRL2 and a CDRL3 comprising the amino acid sequence of SEQ ID NOS: 85, 86, and 87, respectively. In some embodiments, the antibody or antigen-binding fragment of the provided CAR contains a V_{H} region that has a CDRH1, a CDRH2 and a CDRH3 comprising the amino acid sequence of SEQ ID NOS: 82, 83 and 84, respectively and a V_{L} region that contains a CDRL1, a CDRL2 and a CDRL3 comprising the amino acid sequence of SEQ ID NOS: 88, 89 and 87, respectively. In some embodiments, the V_{H} region comprises the sequence set forth in SEQ ID NO:21 and the V_{L} region comprises the sequence set forth in SEQ ID NO:22. In some embodiments, the V_{H} region comprises the sequence set forth in SEQ ID NO:21 and the V_{L} region comprises the sequence set forth in SEQ ID NO:63. In some embodiments, the antibody or antigen-binding fragment is a single-chain antibody fragment, such as an scFv. In some embodiments, the scFv comprises the sequence of amino acids set forth in SEQ ID NO:1 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:1. In some embodiments, the scFv is encoded by a nucleotide sequence set forth in SEQ ID NO:257 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:257. In some embodiments, the scFv comprises the sequence of amino acids set forth in SEQ ID NO:2 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:2. n some embodiments, the scFv is encoded by a nucleotide sequence set forth in SEQ ID NO:258 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:258.

Among a provided anti-GPRC5D CAR is a CAR in which the antibody or antigen-binding fragment contains a V_{H} region comprising the sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and contains a V_{L} region comprising the sequence set forth in SEQ ID NO:24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24. In some embodiments, the provided CAR is a CAR in which the antibody or antigen-binding fragment contains a V_{H} region comprising the sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and contains a V_{L} region comprising the sequence set forth in SEQ ID NO:64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:64. In some embodiments, the antibody or antigen-binding fragment of the provided CAR contains a V_{H} region that has a CDRH1, a CDRH2 and a CDRH3 comprising the amino acid sequence of SEQ ID NOS: 90, 91, 92, respectively and a V_{L} region that has a CDRL1, a CDRL2 and a CDRL3 comprising the amino acid sequence of SEQ ID NOS:100, 101 and 102, respectively. In some embodiments, the antibody or antigen-binding fragment of the provided CAR contains a V_{H} region that has a CDRH1, a CDRH2 and a CDRH3 comprising the amino acid sequence of SEQ ID NOS: 93, 94 and 92, respectively and a V_{L} region that contains a CDRL1, a CDRL2 and a CDRL3 comprising the amino acid sequence of SEQ ID NOS: 100, 101 and 102, respectively. In some embodiments, the antibody or antigen-binding fragment of the provided CAR contains a V_{H} region that has a CDRH1, a CDRH2 and a CDRH3 comprising the amino acid sequence of SEQ ID NOS: 95, 96 and 92, respectively and a V_{L} region that contains a CDRL1, a CDRL2 and a CDRL3 comprising the amino acid sequence of SEQ ID NOS: 100, 101 and 102, respectively. In some embodiments, the antibody or antigen-binding fragment of the provided CAR contains a V_{H} region that has a CDRH1, a CDRH2 and a CDRH3 comprising the amino acid sequence of SEQ ID NOS: 97, 98 and 99, respectively and a V_{L} region that contains a CDRL1, a CDRL2 and a CDRL3 comprising the amino acid sequence of SEQ ID NOS: 103, 104 and 102, respectively. In some embodiments, the V_{H} region comprises the sequence set forth in SEQ ID NO:23 and the V_{L} region comprises the sequence set forth in SEQ ID NO:24. In some embodiments, the V_{H} region comprises the sequence set forth in SEQ ID NO:23 and the V_{L} region comprises the sequence set forth in SEQ ID NO:64. In some embodiments, the antibody or antigen-binding fragment is a single-chain antibody fragment, such as an scFv. In some embodiments, the scFv comprises the sequence of amino acids set forth in SEQ ID NO:3 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:3. In some embodiments, the scFv is encoded by a nucleotide sequence set forth in SEQ ID NO:259 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:259. In some embodiments, the scFv comprises the sequence of amino acids set forth in SEQ ID NO:4 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:4. In some embodiments, the scFv is encoded by a nucleotide sequence set forth in SEQ ID NO:260 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:260.

Among a provided anti-GPRC5D CAR is a CAR in which the antibody or antigen-binding fragment contains a V_{H} region comprising the sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25; and contains a V_{L} region comprising the sequence set forth in SEQ ID NO:26 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:26. In some embodiments, the provided CAR is a CAR in which the antibody or antigen-binding fragment contains a V_{H} region comprising the sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25; and contains a V_{L} region comprising the sequence set forth in SEQ ID NO:65 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:65. In some embodiments, the antibody or antigen-binding fragment of the provided CAR contains a V_{H} region that has a CDRH1, a CDRH2 and a CDRH3 comprising the amino acid sequence of SEQ ID NOS: 105, 106, 107, respectively and a V_{L} region that has a CDRL1, a CDRL2 and a CDRL3 comprising the amino acid sequence of SEQ ID NOS: 115, 116 and 117, respectively. In some embodiments, the antibody or antigen-binding fragment of the provided CAR contains a V_{H} region that has a CDRH1, a CDRH2 and a CDRH3 comprising the amino acid sequence of SEQ ID NOS: 108, 109 and 107, respectively and a V_{L} region that contains a CDRL1, a CDRL2 and a CDRL3 comprising the amino acid sequence of SEQ ID NOS: 115, 116 and 117, respectively. In some embodiments, the antibody or antigen-binding fragment of the provided CAR contains a V_{H} region that has a CDRH1, a CDRH2 and a CDRH3 comprising the amino acid sequence of SEQ ID NOS: 110, 111 and 107, respectively and a V_{L} region that contains a CDRL1, a CDRL2 and a CDRL3 comprising the amino acid sequence of SEQ ID NOS: 115, 116 and 117, respectively. In some embodiments, the antibody or antigen-binding fragment of the provided CAR contains a V_{H} region that has a CDRH1, a CDRH2 and a CDRH3 comprising the amino acid sequence of SEQ ID NOS: 112, 113 and 114, respectively and a V_{L} region that contains a CDRL1, a CDRL2 and a CDRL3 comprising the amino acid sequence of SEQ ID NOS: 118, 119 and 117, respectively. In some embodiments, the V_{H} region comprises the sequence set forth in SEQ ID NO:25 and the V_{L} region comprises the sequence set forth in SEQ ID NO:26. In some embodiments, the V_{H} region comprises the sequence set forth in SEQ ID NO:25 and the V_{L} region comprises the sequence set forth in SEQ ID NO:65. In some embodiments, the antibody or antigen-binding fragment is a single-chain antibody fragment, such as an scFv. In some embodiments, the scFv comprises the sequence of amino acids set forth in SEQ ID NO:5 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:5. In some embodiments, the scFv is encoded by a nucleotide sequence set forth in SEQ ID NO:261 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:261. In some embodiments, the scFv comprises the sequence of amino acids set forth in SEQ ID NO:6 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:6. In some embodiments, the scFv is encoded by a nucleotide sequence set forth in SEQ ID NO:262 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:262.

Among a provided anti-GPRC5D CAR is a CAR in which the antibody or antigen-binding fragment contains a V_{H} region comprising the sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and contains a V_{L} region comprising the sequence set forth in SEQ ID NO:28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:28. In some embodiments, the provided CAR is a CAR in which the antibody or antigen-binding fragment contains a V_{H} region comprising the sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and contains a V_{L} region comprising the sequence set forth in SEQ ID NO:66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:66. In some embodiments, the antibody or antigen-binding fragment of the provided CAR contains a V_{H} region that has a CDRH1, a CDRH2 and a CDRH3 comprising the amino acid sequence of SEQ ID NOS: 120, 121 and 122, respectively and a V_{L} region that has a CDRL1, a CDRL2 and a CDRL3 comprising the amino acid sequence of SEQ ID NOS: 130, 131 and 132, respectively. In some embodiments, the antibody or antigen-binding fragment of the provided CAR contains a V_{H} region that has a CDRH1, a CDRH2 and a CDRH3 comprising the amino acid sequence of SEQ ID NOS: 123, 124 and 122, respectively and a V_{L} region that contains a CDRL1, a CDRL2 and a CDRL3 comprising the amino acid sequence of SEQ ID NOS: 130, 131 and 132, respectively. In some embodiments, the antibody or antigen-binding fragment of the provided CAR contains a V_{H} region that has a CDRH1, a CDRH2 and a CDRH3 comprising the amino acid sequence of SEQ ID NOS: 125, 126 and 122, respectively and a V_{L} region that contains a CDRL1, a CDRL2 and a CDRL3 comprising the amino acid sequence of SEQ ID NOS: 130, 131 and 132, respectively. In some embodiments, the antibody or antigen-binding fragment of the provided CAR contains a V_{H} region that has a CDRH1, a CDRH2 and a CDRH3 comprising the amino acid sequence of SEQ ID NOS: 127, 128 and 129, respectively and a V_{L} region that contains a CDRL1, a CDRL2 and a CDRL3 comprising the amino acid sequence of SEQ ID NOS: 133, 134 and 132, respectively. In some embodiments, the V_{H} region comprises the sequence set forth in SEQ ID NO:27 and the V_{L} region comprises the sequence set forth in SEQ ID NO:28. In some embodiments, the V_{H} region comprises the sequence set forth in SEQ ID NO:27 and the V_{L} region comprises the sequence set forth in SEQ ID NO:66. In some embodiments, the antibody or antigen-binding fragment is a single-chain antibody fragment, such as an scFv. In some embodiments, the scFv comprises the sequence of amino acids set forth in SEQ ID NO:7 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:7. In some embodiments, the scFv is encoded by a nucleotide sequence set forth in SEQ ID NO:263 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:263. In some embodiments, the scFv comprises the sequence of amino acids set forth in SEQ ID NO:8 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:8. In some embodiments, the scFv is encoded by a nucleotide sequence set forth in SEQ ID NO:264 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:264.

Among a provided anti-GPRC5D CAR is a CAR in which the antibody or antigen-binding fragment contains a V_{H} region comprising the sequence set forth in SEQ ID NO:29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:29; and contains a V_{L} region comprising the sequence set forth in SEQ ID NO:30 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:30. In some embodiments, the provided CAR is a CAR in which the antibody or antigen-binding fragment contains a V_{H} region comprising the sequence set forth in SEQ ID NO:29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:29; and contains a V_{L} region comprising the sequence set forth in SEQ ID NO:67 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:67. In some embodiments, the antibody or antigen-binding fragment of the provided CAR contains a V_{H} region that has a CDRH1, a CDRH2 and a CDRH3 comprising the amino acid sequence of SEQ ID NOS: 135, 136 and 137, respectively and a V_{L} region that has a CDRL1, a CDRL2 and a CDRL3 comprising the amino acid sequence of SEQ ID NOS: 145, 146 and 147, respectively. In some embodiments, the antibody or antigen-binding fragment of the provided CAR contains a V_{H} region that has a CDRH1, a CDRH2 and a CDRH3 comprising the amino acid sequence of SEQ ID NOS: 138, 139 and 137, respectively and a V_{L} region that contains a CDRL1, a CDRL2 and a CDRL3 comprising the amino acid sequence of SEQ ID NOS: 145, 146 and 147, respectively. In some embodiments, the antibody or antigen-binding fragment of the provided CAR contains a V_{H} region that has a CDRH1, a CDRH2 and a CDRH3 comprising the amino acid sequence of SEQ ID NOS: 140, 141 and 137, respectively and a V_{L} region that contains a CDRL1, a CDRL2 and a CDRL3 comprising the amino acid sequence of SEQ ID NOS: 145, 146 and 147, respectively. In some embodiments, the antibody or antigen-binding fragment of the provided CAR contains a V_{H} region that has a CDRH1, a CDRH2 and a CDRH3 comprising the amino acid sequence of SEQ ID NOS: 142, 143 and 144, respectively and a V_{L} region that contains a CDRL1, a CDRL2 and a CDRL3 comprising the amino acid sequence of SEQ ID NOS: 148, 149 and 147, respectively. In some embodiments, the V_{H} region comprises the sequence set forth in SEQ ID NO:29 and the V_{L} region comprises the sequence set forth in SEQ ID NO:30. In some embodiments, the V_{H} region comprises the sequence set forth in SEQ ID NO:29 and the V_{L} region comprises the sequence set forth in SEQ ID NO:67. In some embodiments, the antibody or antigen-binding fragment is a single-chain antibody fragment, such as an scFv. In some embodiments, the scFv comprises the sequence of amino acids set forth in SEQ ID NO:9 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:9. In some embodiments, the scFv is encoded by a nucleotide sequence set forth in SEQ ID NO:265 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:265. In some embodiments, the scFv comprises the sequence of amino acids set forth in SEQ ID NO:10 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:10. In some embodiments, the scFv is encoded by a nucleotide sequence set forth in SEQ ID NO:266 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:266.

Among a provided anti-GPRC5D CAR is a CAR in which the antibody or antigen-binding fragment contains a V_{H} region comprising the sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and contains a V_{L} region comprising the sequence set forth in SEQ ID NO:32 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:32. In some embodiments, the provided CAR is a CAR in which the antibody or antigen-binding fragment contains a V_{H} region comprising the sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and contains a V_{L} region comprising the sequence set forth in SEQ ID NO:68 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:68. In some embodiments, the antibody or antigen-binding fragment of the provided CAR contains a V_{H} region that has a CDRH1, a CDRH2 and a CDRH3 comprising the amino acid sequence of SEQ ID NOS: 135, 150 and 151, respectively and a V_{L} region that has a CDRL1, a CDRL2 and a CDRL3 comprising the amino acid sequence of SEQ ID NOS: 157, 158 and 159, respectively. In some embodiments, the antibody or antigen-binding fragment of the provided CAR contains a V_{H} region that has a CDRH1, a CDRH2 and a CDRH3 comprising the amino acid sequence of SEQ ID NOS: 152, 153 and 151, respectively and a V_{L} region that contains a CDRL1, a CDRL2 and a CDRL3 comprising the amino acid sequence of SEQ ID NOS: 157, 158 and 159, respectively. In some embodiments, the antibody or antigen-binding fragment of the provided CAR contains a V_{H} region that has a CDRH1, a CDRH2 and a CDRH3 comprising the amino acid sequence of SEQ ID NOS: 140, 154 and 151, respectively and a V_{L} region that contains a CDRL1, a CDRL2 and a CDRL3 comprising the amino acid sequence of SEQ ID NOS: 157, 158 and 159, respectively. In some embodiments, the antibody or antigen-binding fragment of the provided CAR contains a V_{H} region that has a CDRH1, a CDRH2 and a CDRH3 comprising the amino acid sequence of SEQ ID NOS: 142, 155 and 156, respectively and a V_{L} region that contains a CDRL1, a CDRL2 and a CDRL3 comprising the amino acid sequence of SEQ ID NOS: 160, 161 and 159, respectively. In some embodiments, the V_{H} region comprises the sequence set forth in SEQ ID NO:31 and the V_{L} region comprises the sequence set forth in SEQ ID NO:32. In some embodiments, the V_{H} region comprises the sequence set forth in SEQ ID NO:31 and the V_{L} region comprises the sequence set forth in SEQ ID NO:68. In some embodiments, the antibody or antigen-binding fragment is a single-chain antibody fragment, such as an scFv. In some embodiments, the scFv comprises the sequence of amino acids set forth in SEQ ID NO:11 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:11. In some embodiments, the scFv is encoded by a nucleotide sequence set forth in SEQ ID NO:267 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:267. In some embodiments, the scFv comprises the sequence of amino acids set forth in SEQ ID NO:12 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:12. In some embodiments, the scFv is encoded by a nucleotide sequence set forth in SEQ ID NO:268 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:268.

Among a provided anti-GPRC5D CAR is a CAR in which the antibody or antigen-binding fragment contains a V_{H} region comprising the sequence set forth in SEQ ID NO:33 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:33; and contains a V_{L} region comprising the sequence set forth in SEQ ID NO:34 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:34. In some embodiments, the provided CAR is a CAR in which the antibody or antigen-binding fragment contains a V_{H} region comprising the sequence set forth in SEQ ID NO:33 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:33; and contains a V_{L} region comprising the sequence set forth in SEQ ID NO:69 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:69. In some embodiments, the antibody or antigen-binding fragment of the provided CAR contains a V_{H} region that has a CDRH1, a CDRH2 and a CDRH3 comprising the amino acid sequence of SEQ ID NOS: 162, 163 and 164, respectively and a V_{L} region that has a CDRL1, a CDRL2 and a CDRL3 comprising the amino acid sequence of SEQ ID NOS: 172, 86, 173, respectively. In some embodiments, the antibody or antigen-binding fragment of the provided CAR contains a V_{H} region that has a CDRH1, a CDRH2 and a CDRH3 comprising the amino acid sequence of SEQ ID NOS: 165, 166 and 164, respectively and a V_{L} region that contains a CDRL1, a CDRL2 and a CDRL3 comprising the amino acid sequence of SEQ ID NOS: 172, 86 and 173, respectively. In some embodiments, the antibody or antigen-binding fragment of the provided CAR contains a V_{H} region that has a CDRH1, a CDRH2 and a CDRH3 comprising the amino acid sequence of SEQ ID NOS: 167, 168 and 164, respectively and a V_{L} region that contains a CDRL1, a CDRL2 and a CDRL3 comprising the amino acid sequence of SEQ ID NOS: 172, 86 and 173, respectively. In some embodiments, the antibody or antigen-binding fragment of the provided CAR contains a V_{H} region that has a CDRH1, a CDRH2 and a CDRH3 comprising the amino acid sequence of SEQ ID NOS: 169, 170, 171, respectively and a V_{L} region that contains a CDRL1, a CDRL2 and a CDRL3 comprising the amino acid sequence of SEQ ID NOS: 174, 89 and 175, respectively. In some embodiments, the antibody or antigen-binding fragment of the provided CAR contains a V_{H} region that has a CDRH1, a CDRH2 and a CDRH3 comprising the amino acid sequence of SEQ ID NOS: 169, 170, 171, respectively and a V_{L} region that contains a CDRL1, a CDRL2 and a CDRL3 comprising the amino acid sequence of SEQ ID NOS: 174, 89 and 297, respectively. In some embodiments, the V_{H} region comprises the sequence set forth in SEQ ID NO:33 and the V_{L} region comprises the sequence set forth in SEQ ID NO:34. In some embodiments, the V_{H} region comprises the sequence set forth in SEQ ID NO:33 and the V_{L} region comprises the sequence set forth in SEQ ID NO:69. In some embodiments, the antibody or antigen-binding fragment is a single-chain antibody fragment, such as an scFv. In some embodiments, the scFv comprises the sequence of amino acids set forth in SEQ ID NO:13 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:13. In some embodiments, the scFv is encoded by a nucleotide sequence set forth in SEQ ID NO:269 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:269. In some embodiments, the scFv comprises the sequence of amino acids set forth in SEQ ID NO:14 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:14. In some embodiments, the scFv is encoded by a nucleotide sequence set forth in SEQ ID NO:270 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:270.

Among the antibodies, e.g., antigen-binding fragments, in the provided CARs, are human antibodies. In some embodiments of a provided human anti-GPRC5D antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, CDR-H2 and/or CDR-H3. In some embodiments, the portion of the V_{H} region corresponds to the framework region 1 (FR1), FR2, FR2 and/or FR4. In some embodiments, the portion of the V_{L} region corresponds to the CDR-L1, CDR-L2 and/or CDR-L3. In some embodiments, the portion of the V_{L} region corresponds to the FR1, FR2, FR2 and/or FR4.

In some embodiments, the human antibody, e.g., antigen-binding fragment, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody, e.g., antigen-binding fragment, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody, e.g., antigen-binding fragment, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody, e.g., antigen-binding fragment, contains a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody, e.g., antigen-binding fragment, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody, e.g., antigen-binding fragment, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody, e.g., antigen-binding fragment, contains a framework region that contains human germline gene segment sequences. For example, in some embodiments, the human antibody contains a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment.

b. Spacer

In some embodiments, the recombinant receptor such as a CAR comprising an antibody (e.g., antigen-binding fragment) provided herein, further includes a spacer, which may be or include at least a portion of an immunoglobulin constant region or variant or modified version thereof. In some embodiments, the portion of the immunoglobulin constant region includes a hinge region, e.g., an IgG4 hinge region, and/or a C_{H}1, C_{H}2 or C_{H}3 and/or Fc region. In some embodiments, the constant region or portion is of a human IgG, such as IgG4 or IgG1. In some aspects, the portion of the constant region serves as a spacer region between the antigen-recognition component, such as antigen-binding domain (e.g., scFv) and transmembrane domain. In some embodiments, the length of the spacer is adjusted to optimize the biophysical synapse distance between the CAR-expressing cell, such as a CAR-expressing cell, and the target of the CAR, such as a GPRC5D-expressing tumor cell. In some embodiments, the CAR is expressed by a T-cell, and the length of the spacer is adjusted to a length that is compatible for T-cell activation or to optimize CAR T-cell performance

In some embodiments, the spacer can be of a length that provides for increased responsiveness of the cell following antigen binding, as compared to in the absence of the spacer or as compared to an alternative spacer of a different length (e.g. shorter in length). In some examples, the spacer is at or about 12 amino acids in length or is no more than 12 amino acids in length. In some embodiments, the spacer is at least 100 amino acids in length, such as at least 110, 125, 130, 135, 140, 145, 150, 160, 170, 180, 190, 200, 210, 220, 230, 240, or 250 amino acids in length. Exemplary spacers include those having at least about 10 to 300 amino acids, about 10 to 200 amino acids, about 50 to 175 amino acids, about 50 to 150 amino acids, about 10 to 125 amino acids, about 50 to 100 amino acids, about 100 to 300 amino acids, about 100 to 250 amino acids, about 125 to 250 amino acids, or about 200 to 250 amino acids, and including any integer between the endpoints of any of the listed ranges. In some embodiments, a spacer region is at least about 12 amino acids, at least about 119 amino acids or less, at least about 125 amino acids, at least about 200 amino acids, or at least about 220 amino acids, or at least about 225 amino acids in length.

In some embodiments, the spacer has a length of 125 to 300 amino acids in length, 125 to 250 amino acids in length, 125 to 230 amino acids in length, 125 to 200 amino acids in length, 125 to 180 amino acids in length, 125 to 150 amino acids in length, 150 to 300 amino acids in length, 150 to 250 amino acids in length, 150 to 230 amino acids in length, 150 to 200 amino acids in length, 150 to 180 amino acids in length, 180 to 300 amino acids in length, 180 to 250 amino acids in length, 180 to 230 amino acids in length, 180 to 200 amino acids in length, 200 to 300 amino acids in length, 200 to 250 amino acids in length, 200 to 230 amino acids in length, 230 to 300 amino acids in length, 230 to 250 amino acids in length or 250 to 300 amino acids in length. In some embodiments, the spacer is at least or at least about or is or is about 130, 140, 150, 160, 170, 180, 190, 200, 210, 220, 221, 222, 223, 224, 225, 226, 227, 228 or 229 amino acids in length, or a length between any of the foregoing.

Exemplary spacers include an IgG hinge alone, an IgG hinge linked to one or more of a C_{H}2 and C_{H}3 domain, or IgG hinge linked to the C_{H}3 domain. In some embodiments, the IgG hinge, C_{H}2 and/or C_{H}3 can be derived all or in part from IgG4 or IgG2, such as all or in part from human IgG4 or human IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C_{H}2 region is chimeric and comprises a C_{H}2 region from human IgG4 and human IgG2; and/or the C_{H}3 region is chimeric and comprises a C_{H}3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C_{H}2 region; and a human IgG4 C_{H}3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence set forth in SEQ ID NO: 281 or an N176Q at position 176, in the C_{H}2 region, of the full-length IgG2 Fc sequence set forth in SEQ ID NO: 282. In some embodiments, the spacer is or comprises an IgG4/2 chimeric hinge or a modified IgG4 hinge; an IgG2/4 chimeric C_{H}2 region; and an IgG4 C_{H}3 region. In some embodiments, the spacer is about 228 amino acids in length. In some embodiments, the spacer is set forth in SEQ ID NO: 17. In some embodiments, the spacer comprises the amino acid sequence

(SEQ ID NO: 17)
ESKYGPPCPPCPAPPVAGPSVFLFPPKPKDTLMISRTPEVTCVVVDVS
QEDPEVQFNWYVDGVEVHNAKTKPREEQFQSTYRVVSVLTVLHQDWLN
GKEYKCKVSNKGLPSSIEKTISKAKGQPREPQVYTLPPSQEEMTKNQV
SLTCLVKGFYPSDIAVEWESNGQPENNYKTTPPVLDSDGSFFLYSRLT
VDKSRWQEGNVFSCSVMHEALHNHYTQKSLSLSLGK

In some embodiments, the spacer is encoded by a polynucleotide that has been optimized for codon expression and/or to eliminate splice sites such as cryptic splice sites. In some embodiments, the coding sequence for the spacer comprises the nucleic acid sequence set forth in SEQ ID NO: 74. In some embodiments, the coding sequence for the spacer comprises the nucleic acid sequence set forth in SEQ ID NO: 73. In some embodiments, the coding sequence for the spacer comprises the nucleic acid sequence set forth in SEQ ID NO: 283. In some embodiments, the coding sequence for the spacer comprises the nucleic acid sequence set forth in SEQ ID NO: 284.

Additional exemplary spacers include, but are not limited to, those described in Hudecek et al. (2013) Clin. Cancer Res., 19:3153, Hudecek et al. (2015) Cancer Immunol. Res., 3(2):125-135, or international patent application publication number WO2014031687. In some embodiments, the nucleotide sequence of the spacer is optimized to reduce RNA heterogeneity upon expression. In some embodiments, the nucleotide sequence of the spacer is optimized to reduce cryptic splice sites or reduce the likelihood of a splice event at a splice site.

In some embodiments, the spacer has the amino acid sequence set forth in SEQ ID NO:15, and is encoded by the polynucleotide sequence set forth in SEQ ID NO:285. In some embodiments, the spacer has the amino acid sequence set forth in SEQ ID NO:16. In some embodiments, the spacer has the amino acid sequence set forth in SEQ ID NO:286. In some embodiments, the spacer has the amino acid sequence set forth in SEQ ID NO: 288, and is encoded by the polynucleotide sequence set forth in SEQ ID NO: 287. In some embodiments, the spacer has an amino acid sequence set forth in SEQ ID NO: 17, encoded by the polynucleotide sequence set forth in SEQ ID NO: 73, 74, 283 or 284 or a polynucleotide that exhibits at least 85%, 86%, 87%, 88%, 89%, 90%, 91%, 92%, 93%, 94%, 95%, 96%, 97%, 98%, 99% or more sequence identity to SEQ ID NO: 73, 74, 283 or 284.

In some embodiments, the spacer has an amino acid sequence that exhibits at least 85%, 86%, 87%, 88%, 89%, 90%, 91%, 92%, 93%, 94%, 95%, 96%, 97%, 98%, 99% or more sequence identity to SEQ ID NO: 17, encoded by a polynucleotide that has been optionally optimized for codon usage and/or to reduce RNA heterogeneity. Methods to reduce RNA heterogeneity, such as by removing cryptic splice donor and/or acceptor sites, are described below, such as in Section I.B.2.b. Observations have shown that cryptic splice donor and/or acceptor sites are present in the spacer region of certain immunoglobulin spacers when present in a CAR. In some embodiments, the spacer in a provided CAR is encoded by a polynucleotide in which one or more cryptic splice donor and/or acceptor sites are eliminated and/or are modified to reduce heterogeneity of the RNA transcribed from the construct, such as mRNA, following expression in a cell. In some embodiments, the spacer is encoded by the nucleotide sequence set forth in SEQ ID NO:74 (also set forth in SEQ ID NO:48). In some embodiments, the spacer is encoded by the nucleotide sequence set forth in SEQ ID NO:283. In some embodiments, the spacer is encoded by the nucleotide sequence set forth in SEQ ID NO:284. In some embodiments, the spacer is encoded by the nucleotide sequence set forth in SEQ ID NO:305.

c. Transmembrane Domain and Intracellular Signaling Components

The antigen-recognition component generally is linked to one or more intracellular signaling components, such as signaling components that mimic activation through an antigen receptor complex, such as a TCR complex, in the case of a CAR, and/or signal via another cell surface receptor. Thus, in some embodiments, a GPRC5D-binding molecule (e.g., antibody or antigen binding fragment thereof) is linked to one or more transmembrane domains such as those described herein and intracellular signaling domains comprising one or more intracellular components such as those described herein. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. For example, the transmembrane domain can be a CD28 transmembrane domain that comprises the sequence of amino acids set forth in SEQ ID NO: 18, encoded by the nucleic acid sequence set forth in SEQ ID NO: 55 or SEQ ID NO: 56. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the CAR.

The receptor, e.g., the CAR, generally includes an intracellular signaling region comprising at least one intracellular signaling component or components. In some embodiments, the receptor includes an intracellular component or signaling domain of a TCR complex, such as a TCR CD3 chain that mediates T-cell activation and cytotoxicity, e.g., CD3 zeta (CD3-ζ) chain. Thus, in some aspects, the GPRC5D-binding antibody is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

In some embodiments, upon ligation of the CAR, the cytoplasmic domain or intracellular signaling domain of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such classes of cytoplasmic signaling sequences.

In some aspects, the CAR includes a primary cytoplasmic signaling sequence that regulates primary stimulation and/or activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling region in the CAR contain(s) a cytoplasmic signaling domain, portion thereof, or sequence derived from CD3 zeta. In some embodiments the CD3 zeta comprises the sequence of amino acids set forth in SEQ ID NO: 20, encoded by the nucleic acid sequence set forth in SEQ ID NO: 57 or SEQ ID NO: 58.

In some embodiments, the CAR includes a signaling domain (e.g., an intracellular or cytoplasmic signaling domain) and/or transmembrane portion of a costimulatory molecule, such as a T cell costimulatory molecule. Exemplary costimulatory molecules include CD28, 4-1BB, OX40, DAP10, and ICOS. For example, a costimulatory molecule can be derived from 4-1BB and can comprise the amino acid sequence set forth in SEQ ID NO: 19, encoded by the nucleotide sequence set forth in SEQ ID NO: 59 or SEQ ID NO: 60. In some aspects, the same CAR includes both the stimulatory or activating components (e.g., cytoplasmic signaling sequence) and costimulatory components.

In some embodiments, the stimulatory or activating components are included within one CAR, whereas the costimulatory component is provided by another CAR recognizing another antigen. In some embodiments, the CARs include activating or stimulatory CARs, and costimulatory CARs, both expressed on the same cell (see WO 2014/055668). In some aspects, the GPRC5D-targeting CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December, 2013), such as a CAR recognizing an antigen other than GPRC5D, whereby a stimulatory or an activating signal delivered through the GPRC5D-targeting CAR is diminished or inhibited by binding of the inhibitory CAR to its ligand, e.g., to reduce off-target effects.

In certain embodiments, the intracellular signaling region comprises a CD28 transmembrane and signaling domain linked to a CD3 (e.g., CD3-zeta) intracellular domain. In some embodiments, the intracellular signaling domain comprises a chimeric CD28 and 4-1BB (CD137; TNFRSF9) co-stimulatory domains, linked to a CD3 zeta intracellular domain.

In some embodiments, the CAR encompasses one or more, e.g., two or more, costimulatory domains and a stimulatory or an activation domain, e.g., primary activation domain, in the cytoplasmic portion. Exemplary CARs include intracellular components of CD3-zeta, CD28, and 4-1BB.

In some embodiments, provided embodiments of anti-GPRC5D CAR contains an extracellular antigen-binding domain containing any of the anti-GPRC5D antibody or antigen-binding fragments described herein, such as in Section I.1a; a spacer comprising an IgG4/2 chimeric hinge or a modified IgG4 hinge; an IgG2/4 chimeric C_{H}2 region; and an IgG4 C_{H}3 region, such as one that is about 228 amino acids in length, or a spacer set forth in SEQ ID NO:17, such as encoded by the nucleotide sequence set forth in any of SEQ ID NOS: 73, 74, 283 or 284; a transmembrane domain, such as a transmembrane domain from a human CD28; and an intracellular signaling region comprising a cytoplasmic signaling domain of a CD3-zeta (CD3ζ) chain and an intracellular signaling domain of a T cell costimulatory molecule. Also provided are polynucleotides encoding such a chimeric antigen receptor. In some embodiments, the transmembrane domain is or comprises the sequence set forth in SEQ ID NO:18. In some embodiments, the intracellular signaling domain of a T cell costimulatory molecule is an intracellular signaling domain of human CD28, human 4-1BB or human ICOS or a signaling portion thereof. In particular embodiments, the intracellular signaling domain is an intracellular signaling domain of human 4-1BB. In some embodiments, the intracellular signaling domain is or comprises the sequence set forth in SEQ ID NO:19. In some embodiments, the cytoplasmic signaling domain is a human CD3-zeta cytoplasmic signaling domain, such as set forth in SEQ ID NO:20. In some embodiments, the intracellular signaling region comprises the sequences set forth in SEQ ID NO:20 and SEQ ID NO:19.

In some embodiments, provided embodiments of an anti-GPRC5D CARs has an amino acid sequence set forth in SEQ ID NO:289, or an amino acid sequence that is at least at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO:289. In some embodiments, provided embodiments of an anti-GPRC5D CAR is encoded nucleotide sequence set forth in SEQ ID NO:290 or a nucleotide sequence that has at least at or about 90%, at or about 91%, at or about 92%, at or about 93%, at or about 94%, at or about 95%, at or about 96%, at or about 97%, at or about 98% or at or about 99% sequence identity to the sequence set forth in any of SEQ ID NO:290.

2. Exemplary Features

In some of any of the provided embodiments, the anti-GPRC5D CAR and/or anti-GPRC5D antigen-binding domain, specifically binds to GPRC5D, such as GPRC5D on the surface of a multiple myeloma plasma cell. In any of the embodiments, an antibody or antigen binding fragment, in the provided CARs, that specifically binds GPRC5D. In some embodiments binding can be to a human GPRC5D, a mouse GPRC5D protein, or a non-human primate (e.g., cynomolgus monkey) GPRC5D protein. In some embodiments, among provided anti-GPRC5D CAR and/or anti-GPRC5D antigen-binding domain are those that bind human GPRC5D protein. The observation that an antibody or other binding molecule binds to GPRC5D protein or specifically binds to GPRC5D protein does not necessarily mean that it binds to a GPRC5D protein of every species. For example, in some embodiments, features of binding to GPRC5D protein, such as the ability to specifically bind thereto and/or to compete for binding thereto with a reference antibody, and/or to bind with a particular affinity or compete to a particular degree, in some embodiments, refers to the ability with respect to a human GPRC5D protein and the antibody may not have this feature with respect to a GPRC5D protein of another species, such as mouse.

In some embodiments, the antibodies specifically bind to human GPRC5D protein, such as to an epitope or region of human GPRC5D protein, such as the human BCMA protein comprising the amino acid sequence of SEQ ID NO:49 (Uniprot Q9NZD1), or an allelic variant or splice variant thereof.

In one embodiment, the extent of binding of an anti-GPRC5D antibody or antigen-binding domain or CAR to an unrelated, non-GPRC5D protein, such as a non-human GPRC5D protein or other non-GPRC5D protein, is less than at or about 10% of the binding of the antibody or antigen-binding domain or CAR to human GPRC5D protein or human membrane-bound GPRC5D as measured, e.g., by a radioimmunoassay (RIA). In some embodiments, among the antibodies or antigen-binding domains in the provided CARs, are antibodies or antigen-binding domains or CARs in which binding to mouse GPRC5D protein is less than or at or about 10% of the binding of the antibody to human GPRC5D protein. In some embodiments, among the antibodies or antigen-binding domains in the provided CARs, are antibodies in which binding to cynomolgus monkey GPRC5D protein is less than or at or about 10% of the binding of the antibody to human GPRC5D protein. In some embodiments, among the antibodies or antigen-binding domains in the provided CARs, are antibodies in which binding to cynomolgus monkey GPRC5D protein and/or a mouse GPRC5D protein is similar to or about the same as the binding of the antibody to human GPRC5D protein.

In some embodiments, the antibodies, in the provided CARs, are capable of binding GPRC5D protein, such as human GPRC5D protein, with at least a certain affinity, as measured by any of a number of known methods. In some embodiments, the affinity is represented by an equilibrium dissociation constant (K_{D}); in some embodiments, the affinity is represented by EC₅₀.

A variety of assays are known for assessing binding affinity and/or determining whether a binding molecule (e.g., an antibody or fragment thereof) specifically binds to a particular ligand (e.g., an antigen, such as a GPRC5D protein). It is within the level of a skilled artisan to determine the binding affinity of a binding molecule, e.g., an antibody, for an antigen, e.g., GPRC5D, such as human GPRC5D or cynomolgus GPRC5D or mouse GPRC5D, such as by using any of a number of binding assays that are well known in the art. For example, in some embodiments, a BIAcore® instrument can be used to determine the binding kinetics and constants of a complex between two proteins (e.g., an antibody or fragment thereof, and an antigen, such as a GPRC5D protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

In some embodiments, the binding molecule, e.g., antibody or fragment thereof or antigen-binding domain of a CAR, binds, such as specifically binds, to an antigen, e.g., a GPRC5D protein or an epitope therein, with an affinity or K_{A} (i.e., an equilibrium association constant of a particular binding interaction with units of 1/M; equal to the ratio of the on-rate [kₒₙ or k_{d}] to the off-rate [k_{off} or k_{d}] for this association reaction, assuming bimolecular interaction) equal to or greater than 10⁵ M⁻¹. In some embodiments, the antibody or fragment thereof or antigen-binding domain of a CAR exhibits a binding affinity for the peptide epitope with a K_{D} (i.e., an equilibrium dissociation constant of a particular binding interaction with units of M; equal to the ratio of the off-rate [k_{off} or k_{d}] to the on-rate [kₒₙ or k_{d}] for this association reaction, assuming bimolecular interaction) of equal to or less than 10⁻⁵ M. For example, the equilibrium dissociation constant K_{D} ranges from 10⁻⁵ M to 10⁻¹³ M, such as 10⁻⁷ M to 10⁻¹¹ M, 10⁻⁸M to 10⁻¹⁰ M, or 10⁻⁹ M to 10⁻¹⁰ M. The on-rate (association rate constant; kₒₙ or kₐ; units of 1/Ms) and the off-rate (dissociation rate constant; k_{off} or k_{d}; units of 1/s) can be determined using any of the assay methods known in the art, for example, surface plasmon resonance (SPR).

In some embodiments, the binding affinity (EC₅₀) and/or the dissociation constant of the antibody (e.g. antigen-binding fragment) or antigen-binding domain of a CAR to about GPRC5D protein, such as human GPRC5D protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, is from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC₅₀) and/or the equilibrium dissociation constant, K_{D}, of the antibody to a GPRC5D protein, such as human GPRC5D protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a GPRC5D protein, such as human GPRC5D protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a K_{A} of up to 10⁷ M⁻¹, up to 10⁶ M⁻¹, up to 10⁵ M⁻¹. In some cases, a binding molecule (e.g. antibody or fragment thereof) that exhibits high affinity binding to a particular epitope interacts with such epitope with a K_{A} of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In some embodiments, the binding affinity (EC₅₀) and/or the equilibrium dissociation constant, K_{D}, of the binding molecule, e.g., anti-GPRC5D antibody or fragment thereof or antigen-binding domain of a CAR, to a GPRC5D protein, is from or from about 0.01 nM to about 1 μM, 0.1 nM to 1 μM, 1 nM to 1 μM, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC₅₀) and/or the dissociation constant of the equilibrium dissociation constant, K_{D}, of the binding molecule, e.g., anti-GPRC5D antibody or fragment thereof or antigen-binding domain of a CAR, to a GPRC5D protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody.

In some embodiments, the binding affinity of a binding molecule, such as an anti-GPRC5D antibody or antigen-binding domain of a CAR, for different antigens, e.g., GPRC5D proteins from different species can be compared to determine the species cross-reactivity. For example, species cross-reactivity can be classified as high cross reactivity or low cross reactivity. In some embodiments, the equilibrium dissociation constant, K_{D}, for different antigens, e.g., GPRC5D proteins from different species such as human, cynomolgus monkey or mouse, can be compared to determine species cross-reactivity. In some embodiments, the species cross-reactivity of an anti-GPRC5D antibody or antigen-binding domain of a CAR can be high, e.g., the anti-GPRC5D antibody binds to human GPRC5D and a species variant GPRC5D to a similar degree, e.g., the ratio of K_{D} for human GPRC5D and K_{D} for the species variant GPRC5D is or is about 1. In some embodiments, the species cross-reactivity of an anti-GPRC5D antibody or antigen-binding domain of a CAR can be low, e.g., the anti-GPRC5D antibody has a high affinity for human GPRC5D but a low affinity for a species variant GPRC5D, or vice versa. For example, the ratio of K_{D} for the species variant GPRC5D and K_{D} for the human GPRC5D is more than 10, 15, 20, 25, 30, 40, 50, 60, 70, 80, 90, 100, 200, 500, 1000, 2000 or more, and the anti-GPRC5D antibody has low species cross-reactivity. The degree of species cross-reactivity can be compared with the species cross-reactivity of a known antibody, such as a reference antibody.

Among the provided CARs are CARs that exhibit antigen-dependent activity or signaling, i.e. signaling activity that is measurably absent or at background levels in the absence of antigen, e.g. GPRC5D. Thus, in some aspects, provided CARs do not exhibit, or exhibit no more than background or a tolerable or low level of, tonic signaling or antigen-independent activity or signaling in the absence of antigen, e.g. GPRC5D, being present. In some embodiments, the provided anti-GPRC5D CAR-expressing cells exhibit biological activity or function, including cytotoxic activity, cytokine production, and ability to proliferate.

In some embodiments, biological activity or functional activity of a chimeric receptor, such as cytotoxic activity, can be measured using any of a number of known methods. The activity can be assessed or determined either in vitro or in vivo. In some embodiments, activity can be assessed once the cells are administered to the subject (e.g., human) Parameters to assess include specific binding of an engineered or natural T cell or other immune cell to antigen, e.g., in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as interleukin-2 (IL-2), interferon-gamma (IFNγ), interleukin-4 (IL-4), TNF-alpha (TNFα), interleukin-6 (IL-6), interleukin-10 (IL-10), interleukin-12 (IL-12), granulocyte-macrophage colony-stimulating factor (GM-CSF), CD107a, and/or TGF-beta (TGFβ). Assays to measure cytokines are well known in the art, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample. In some aspects, the biological activity is measured by assessing clinical outcome, such as reduction in tumor burden or load.

In some aspects, a reporter cell line can be employed to monitor antigen-independent activity and/or tonic signaling through anti-GPRC5D CAR-expressing cells. In some embodiments, a T cell line, such as a Jurkat cell line, contains a reporter molecule, such as a fluorescent protein or other detectable molecule, such as a red fluorescent protein, expressed under the control of the endogenous Nur77 transcriptional regulatory elements. In some embodiments, the Nur77 reporter expression is cell intrinsic and dependent upon signaling through a recombinant reporter containing a primary activation signal in a T cell, a signaling domain of a T cell receptor (TCR) component, and/or a signaling domain comprising an immunoreceptor tyrosine-based activation motif (ITAM), such as a CD3ζ chain. Nur77 expression is generally not affected by other signaling pathways such as cytokine signaling or toll-like receptor (TLR) signaling, which may act in a cell extrinsic manner and may not depend on signaling through the recombinant receptor. Thus, only cells that express the exogenous recombinant receptor, e.g. anti-GPRC5D CAR, containing the appropriate signaling regions is capable of expressing Nur77 upon stimulation (e.g., binding of the specific antigen). In some cases, Nur77 expression also can show a dose-dependent response to the amount of stimulation (e.g., antigen).

In some embodiments, the provided anti-GPRC5D CARs exhibit improved expression on the surface of cells, such as compared to an alternative CAR that has an identical amino acid sequence but that is encoded by non-splice site eliminated and/or a non-codon-optimized nucleotide sequence. In some embodiments, the expression of the recombinant receptor on the surface of the cell can be assessed. Approaches for determining expression of the recombinant receptor on the surface of the cell may include use of chimeric antigen receptor (CAR)-specific antibodies (e.g., Brentjens et al., Sci. Transl. Med. 2013 March; 5(177): 177ra38), Protein L (Zheng et al., J. Transl. Med. 2012 February; 10:29), epitope tags, and monoclonal antibodies that specifically bind to a CAR polypeptide (see international patent application Pub. No. WO2014190273). In some embodiments, the expression of the recombinant receptor on the surface of the cell, e.g., primary T cell, can be assessed, for example, by flow cytometry, using binding molecules that can bind to the recombinant receptor or a portion thereof that can be detected. In some embodiments, the binding molecules used for detecting expression of the recombinant receptor an anti-idiotypic antibody, e.g., an anti-idiotypic agonist antibody specific for a binding domain, e.g., scFv, or a portion thereof. In some embodiments, the binding molecule is or comprises an isolated or purified antigen, e.g., recombinantly expressed antigen.

A. Dual Antigen-Targeting Chimeric Antigen Receptor(s)

Also provided are polynucleotides encoding the chimeric antigen receptors and/or portions, e.g., chains, thereof. Among the provided polynucleotides are those encoding chimeric antigen receptors that bind to BCMA and GPRC5D (e.g., antigen-binding fragment) described herein, such as a chimeric antigen receptor comprising an anti-BCMA scFv and an anti-GPRC5D scFv (a “single stalk” chimeric antigen receptor). The polynucleotides may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide. In some cases, the polynucleotide may comprise the sequence set forth in SEQ ID NO:317.

In some cases, the polynucleotide encoding the GPRC5D-binding and BCMA-binding molecules contains a signal sequence that encodes a signal peptide, in some cases encoded upstream of the nucleic acid sequences encoding the GPRC5D-binding and BCMA-binding molecules, or joined at the 5′ terminus of the nucleic acid sequences encoding the antigen-binding domains. In some cases, the polynucleotide containing nucleic acid sequences encoding the GPRC5D-binding and BCMA-binding receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of the IgG kappa chain set forth in SEQ ID NO: 271, or encoded by the nucleotide sequence set forth in SEQ ID NO: 272 or 273-276. In some aspects, a non-limiting exemplary signal peptide includes a signal peptide of a GMCSFR alpha chain set forth in SEQ ID NO:278 and encoded by the nucleotide sequence set forth in SEQ ID NO:277. In some aspects, a non-limiting exemplary signal peptide includes a signal peptide of a CD8 alpha signal peptide set forth in SEQ ID NO:279. In some aspects, a non-limiting exemplary signal peptide includes a signal peptide of a CD33 signal peptide set forth in SEQ ID NO:280. In some cases, the polynucleotide encoding the GPRC5D-binding and BCMA-binding receptor can contain nucleic acid sequence encoding additional molecules, such as a surrogate marker or other markers, or can contain additional components, such as promoters, regulatory elements and/or multicistronic elements. In some embodiments, the nucleic acid sequence encoding the GPRC5D-binding and BCMA-binding receptor can be operably linked to any of the additional components. In some cases, the anti-GPRC5D scFv and the anti-BCMA scFv are separated by a nucleotide sequence encoding a flexible linker, such as the nucleotide sequence set forth in SEQ ID NO:320. In some cases, the construct comprising a GPRC5D-binding and BCMA-binding receptor further comprises a 4-1BB costimulatory domain (SEQ ID NO:60, encoding SEQ ID NO:19).

In some embodiments, cells express a CAR that binds both GPRC5D and BCMA as a therapeutic agent against MM plasma cells. In some embodiments, the polynucleotide constructs are codon diverged to improve expression of one or more of the scFvs encoded by the polynucleotide.

In some embodiments, among CARs provided herein are those encoded by polynucleotides that are optimized, or contain certain features designed for optimization, such as for codon usage, to reduce RNA heterogeneity and/or to modify, e.g., increase or render more consistent among cell product lots, expression, such as surface expression, of the encoded receptor, such as described in Section IB below. Thus, also provided are cells expressing the recombinant receptors encoded by the polynucleotides provided herein and uses thereof in adoptive cell therapy, such as treatment of diseases and disorders associated with GPRC5D and/or BCMA expression, e.g., multiple myeloma.

Also provided are cells, such as T cells, engineered to express a polynucleotide encoding a provided polynucleotide, including polynucleotides encoding a first and second scFv, and compositions containing such cells. In some embodiments, the polynucleotide constructs are modified as described in Section IB below.

B. Polynucleotides Encoding Recombinant Receptor(s)

Also provided are polynucleotides encoding the chimeric antigen receptors and/or portions, e.g., chains, thereof. Among the provided polynucleotides are those encoding the anti-GPRC5D chimeric antigen receptors (e.g., antigen-binding fragment) described herein. The polynucleotides may include those encompassing natural and/or non-naturally occurring nucleotides and bases, e.g., including those with backbone modifications. The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some cases, the polynucleotide encoding the GPRC5D-binding receptor contains a signal sequence that encodes a signal peptide, in some cases encoded upstream of the nucleic acid sequences encoding the GPRC5D-binding receptor, or joined at the 5′ terminus of the nucleic acid sequences encoding the antigen-binding domain. In some cases, the polynucleotide containing nucleic acid sequences encoding the GPRC5D-binding receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some aspects, non-limiting exemplary signal peptide include a signal peptide of the IgG kappa chain set forth in SEQ ID NO: 271, or encoded by the nucleotide sequence set forth in SEQ ID NO: 272 or 273-276. In some aspects, a non-limiting exemplary signal peptide includes a signal peptide of a GMCSFR alpha chain set forth in SEQ ID NO:278 and encoded by the nucleotide sequence set forth in SEQ ID NO:277. In some aspects, a non-limiting exemplary signal peptide includes a signal peptide of a CD8 alpha signal peptide set forth in SEQ ID NO:279. In some aspects, a non-limiting exemplary signal peptide includes a signal peptide of a CD33 signal peptide set forth in SEQ ID NO:280. In some cases, the polynucleotide encoding the GPRC5D-binding receptor can contain nucleic acid sequence encoding additional molecules, such as a surrogate marker or other markers, or can contain additional components, such as promoters, regulatory elements and/or multicistronic elements. In some embodiments, the nucleic acid sequence encoding the GPRC5D-binding receptor can be operably linked to any of the additional components.

Also provided herein are polynucleotide constructs encoding a first CAR having a first antigen binding domain and a second CAR having a second antigen binding domain, including polynucleotide constructs that are codon diverged. In some embodiments, the first CAR and the second CAR encoded by a polynucleotide construct are capable of binding to different antigens. In some embodiments, a polynucleotide construct encodes a first CAR capable of binding GPRC5D, such as any CAR as described herein, and a second CAR capable of binding BCMA. Exemplary CARs binding BCMA are described herein, such as in Section II. In some embodiments, cells express an anti-GPRC5D CAR and an anti-BCMA CAR as a therapeutic agent against MM plasma cells. In some embodiments, the polynucleotide constructs are codon diverged to improve expression of one or more of the CARs encoded by the polynucleotide.

In some embodiments, among CARs provided herein are those encoded by polynucleotides that are optimized, or contain certain features designed for optimization, such as for codon usage, to reduce RNA heterogeneity and/or to modify, e.g., increase or render more consistent among cell product lots, expression, such as surface expression, of the encoded receptor. In some embodiments, polynucleotides, encoding GPRC5D-binding cell surface proteins, are modified as compared to a reference polynucleotide, such as to remove cryptic or hidden splice sites, to reduce RNA heterogeneity. In some embodiments, polynucleotides, encoding GPRC5D-binding and BCMA-binding cell surface proteins, are codon optimized, such as for expression in a mammalian, e.g., human, cell, such as in a human T cell. In some aspects, the modified polynucleotides result in in improved, e.g., increased or more uniform or more consistent level of, expression, e.g., surface expression, when expressed in a cell. Such polynucleotides can be utilized in constructs for generation of engineered cells that express the encoded GPRC5D-binding and BCMA-binding cell surface protein. Thus, also provided are cells expressing the recombinant receptors encoded by the polynucleotides provided herein and uses thereof in adoptive cell therapy, such as treatment of diseases and disorders associated with GPRC5D and/or BCMA expression, e.g., multiple myeloma.

Also provided are cells, such as T cells, engineered to express a polynucleotide encoding a provided polynucleotide, including polynucleotides encoding a first and second CAR, and compositions containing such cells. In some embodiments, the polynucleotide constructs are codon optimized for expression in a human cell. In some embodiments, one or more splice donor and/or acceptor sites in a polynucleotide construct is modified to reduce heterogeneity of the RNA transcribed from the construct, such as mRNA, following expression in a cell.

1. Bicistronic Polynucleotides

Provided in some aspects are polynucleotides that encode a first chimeric antigen receptor capable of binding, such as specifically binding, a first antigen, and a second chimeric antigen receptor capable of binding, such as specifically binding, a second antigen. Provided herein are polynucleotides that are bicistronic for expression of multiple CARs, such as an anti-GPRCD CAR and an anti-BCMA CAR. In some embodiments, the polynucleotide can contain a nucleic acid encoding an anti-GPRC5D CAR provided herein and a nucleic acid encoding a second CAR, such as an anti-BCMA CAR, separated by a multicistronic element for expression of both CARs in the same cell. In some aspects, the encoded chimeric antigen receptors, such as those containing BCMA-binding polypeptides or GPRC5D-binding polypeptides, and compositions and articles of manufacture and uses of the same, also are provided and may be for use as a therapeutic agent against MM plasma cells.

Among the BCMA-binding polypeptides and GPRC5D-binding polypeptides are antibodies, such as single-chain antibodies (e.g., antigen binding antibody fragments), or portions thereof. In some examples, the chimeric antigen receptors contain anti-BCMA antibodies or antigen-binding fragments thereof. In some examples, the chimeric antigen receptors contain anti-GPRC5D antibodies or antigen-binding fragments thereof. The provided polynucleotides can be incorporated into constructs, such as deoxyribonucleic acid (DNA) or RNA constructs, such as those that can be introduced into cells for expression of the encoded recombinant anti-BCMA and anti-GPRC5D CARs.

Provided in some aspects are BCMA-binding agents, such as recombinant receptors or chimeric antigen receptors that bind BCMA molecules and polynucleotides encoding BCMA binding cell surface proteins, such as recombinant receptors (e.g., CARs), and cells expressing such receptors. The BCMA-binding cell surface proteins generally contain antibodies (e.g., antigen-binding antibody fragments), and/or other binding peptides that specifically bind to BCMA, such as to BCMA proteins, such as human BCMA protein. In some aspects, the agents bind to an extracellular portion of BCMA.

Provided in some aspects are GPRC5D-binding agents, such as recombinant receptors or chimeric antigen receptors that bind GPRC5D molecules and polynucleotides encoding BCMA binding cell surface proteins, such as recombinant receptors (e.g., CARs), and cells expressing such receptors. The GPRC5D-binding cell surface proteins generally contain antibodies (e.g., antigen-binding antibody fragments), and/or other binding peptides that specifically bind to GPRC5D, such as to GPRC5D proteins, such as human GPRC5D protein. In some aspects, the agents bind to an extracellular portion of GPRC5D.

In some embodiments, the first and/or second chimeric antigen receptors include one or more of an antigen binding domain, spacer, a transmembrane domain, and an intracellular signaling region. In some embodiments, the polynucleotide constructs are codon diverged to improve expression of one or more of the CARs encoded by the polynucleotide. In some embodiments, a nucleotide sequence encoding one or more components or the first and/or second chimeric antigen receptor has been codon diverged. In some embodiments, the codon divergence improves expression of one or more of the chimeric antigen receptors. In some embodiments, codon diverge improves expression of the chimeric antigen receptor encoded by a nucleotide sequence that is 3′ relative to a nucleotide sequence encoding the other chimeric antigen receptor. In some embodiments, the polynucleotide constructs are codon optimized for expression in a human cell. In some embodiments, one or more splice donor and/or acceptor sites in a polynucleotide construct is modified to reduce heterogeneity of the RNA transcribed from the construct, such as mRNA, following expression in a cell.

In some embodiments, provided herein are codon diverged polynucleotide constructs encoding the two CARs. It is observed herein that expression of a CAR encoded by a nucleotide sequence of a polynucleotide construct is reduced compared to the other CAR encoded by a nucleotide sequence of the polynucleotide construct. In some embodiments, the CAR encoded by a nucleotide sequence that is 3′ relative to the other encoded CAR is identified as the “trailing” CAR. Similarly, the CAR encoded by the nucleotide sequence that is 5′ relative to the other encoded CAR is identified as the “leading” CAR. In some embodiments, the “leading” CAR corresponds to the CAR that is expressed N-terminally relative to the other CAR, and the “trailing” CAR corresponds to the CAR that is expressed C-terminally relative to the other CAR.

It is observed herein that expression of the CAR encoded by a nucleotide sequence located 3′ (the “trailing” CAR) relative to the CAR encoded by another nucleotide sequence (the “leading” CAR) is reduced. In some embodiments, it is contemplated that DNA recombination results in loss of the nucleotide sequence encoding the CAR that is located 3′ relative to the nucleotide sequence encoding the other CAR, loss of expression of the CAR encoded by a nucleotide sequence that is 3′ relative to a nucleotide sequence encoding the other CAR, or both.

In some embodiments, polynucleotide constructs provided herein are codon diverged to prevent such loss, such as by codon diverging the nucleotide sequence encoding one of the CAR, e.g. the leading CAR or the trailing CAR. In some embodiments, the nucleotide sequence encoding one of the CARs is codon diverged, such that the nucleotide sequence encoding a first CAR shares no more than 20 base pairs, 15 base pairs, 10 base pairs, or 5 base pairs, of sequence homology with the nucleotide sequencing encoding the second CAR. In some embodiments, the nucleotide sequence encoding the anti-GPRC5D CAR, or components thereof, such as components including a GPRC5D-binding scFv, a spacer, a transmembrane domain, and an intracellular signaling region, is codon diverged.

Such codon diverged polynucleotides can be utilized in constructs for generation of engineered cells that express the encoded GPRC5D-binding and BCMA-binding cell surface protein. Thus, also provided are cells expressing the recombinant receptors encoded by the codon diverged polynucleotides provided herein and uses thereof in adoptive cell therapy, such as treatment of diseases and disorders associated with GPRC5D and/or BCMA expression, e.g., multiple myeloma.

a. Codon Divergence

In any of the provided embodiments, a polynucleotide construct is codon diverged to improve expression of one or more of the CARs encoded by the polynucleotide. The observations herein further demonstrate that expression of multiple CARs, e.g. an anti-GPRC5D CAR and an anti-BCMA CAR, in a cell can be improved by codon diverging a polynucleotide sequence encoding one or more of the CARs. It is found herein that codon divergence of a polynucleotide construct encoding two CARs improves expression of a nucleotide sequence encoding a CAR that is 3′prime (or C-terminal) relative to nucleotide sequence encoding the other CAR, e.g. expression of the trailing CAR is improved by codon divergence.

In some embodiments, provided herein are codon diverged polynucleotide constructs encoding two CARS. It is observed herein that expression of a CAR encoded by a nucleotide sequence of a polynucleotide construct is reduced compared to the other CAR encoded by a nucleotide sequence of the polynucleotide construct. In particular, it is observed herein that the CAR encoded by a nucleotide sequence located 3′ relative to the CAR encoded by another nucleotide sequence is reduced.

In some embodiments, it is contemplated that DNA recombination results in loss of part, or all, of the nucleotide sequence encoding the CAR that is located 3′ relative to the nucleotide sequence encoding the other CAR, loss of expression of the CAR encoded by the nucleotide sequence that is 3′ relative to the nucleotide sequence encoding the other CAR, or both. It is contemplated that DNA recombination results in this loss because of sequence homology between the nucleotide sequences encoding the two CARs.

In some embodiments, polynucleotide constructs provided herein are codon diverged to prevent such loss, such as by codon diverging the nucleotide sequence encoding one of the CARs. In some embodiments, the nucleotide sequence encoding one of the CARs is codon diverged to reduce the homology between the nucleotide sequences encoding the two CARs. In some embodiments, the reduction in homology between the nucleotide sequences encoding the two CARS reduces the probability of homologous recombination and loss of part, or all, of the nucleotide sequencing encoding the trailing CAR. In some embodiments, codon divergence includes modifying the nucleotide sequence of the leading CAR to prevent loss of the sequence encoding the trailing CAR, loss of expression of the trailing CAR, or both. In some embodiments, codon divergence includes modifying the nucleotide sequence of the trailing CAR to prevent loss of the sequence encoding the trailing CAR, loss of expression of the trailing CAR, or both.

In some embodiments, the nucleotide sequence encoding one of the CARs is codon diverged, such that the nucleotide sequence encoding a first CAR shares no more than about 20 base pairs, about 15 base pairs, about 10 base pairs, or about 5 base pairs, of sequence homology with the nucleotide sequencing encoding the second CAR. In some embodiments, nucleotide sequencing encoding one of the CARs is codon diverged, such that the nucleotide sequences encoding the two CARs share no more than about 20, no more than about 15, no more than about 10, or no more than about 5 consecutive, identical bases in any one sequence found within the nucleotide sequences encoding the two CARs.

In some embodiments, nucleotide sequences encoding one or more of the following CAR components is codon diverged: (a) an antigen binding domain; (b) a spacer; (c) a transmembrane domain; (d) an intracellular signaling region. In some embodiments, the nucleotide sequences encoding one or more of components (b) through (d) is codon diverged, resulting in one or more components of the first CAR having a different nucleotide sequence than that of the same component of the second CAR. In some embodiments, the nucleotide sequence encoding one or more of components (b) through (d) in the first CAR is different than that of the nucleotide sequence encoding the same component in the second CAR, but the nucleotide sequence encoding the component in the first CAR and the nucleotide sequence encoding the same component in the second CAR encode the same amino acid sequence.

In some embodiments, the nucleotide sequence encoding a spacer in a first CAR is given by SEQ ID NO:305 and the nucleotide sequence encoding the same spacer in a second CAR is given by SEQ ID NO:74. In some embodiments, the spacer is given by the amino acid sequence set forth in SEQ ID NO:17. In some embodiments, the nucleotide sequence encoding a transmembrane domain in a first CAR is given by SEQ ID NO:307 and the nucleotide sequence encoding the same transmembrane domain in a second CAR is given by SEQ ID NO:56. In some embodiments, the transmembrane domain is given by the amino acid sequence set forth in SEQ ID NO:18. In some embodiments, the nucleotide sequence encoding a 4-1BB endodomain of the intracellular signaling region in a first CAR is given by SEQ ID NO:308 and the nucleotide sequence encoding the same 4-1BB endodomain of the intracellular signaling region in a second CAR is given by SEQ ID NO:60. In some embodiments, the 4-1BB endodomain is given by the amino acid sequence set forth in SEQ ID NO:19. In some embodiments, the nucleotide sequence encoding a CD3zeta endodomain of the intracellular signaling region in a first CAR is given by SEQ ID NO:309 and the nucleotide sequence encoding the same CD3zeta endodomain of the intracellular signaling region in a second CAR is given by SEQ ID NO:58. In some embodiments, the CD3zeta endodomain is given by the amino acid sequence set forth in SEQ ID NO:20.

In some embodiments, the antigen binding domain of the first CAR binds a different antigen than the antigen binding domain of the second CAR. In some embodiments, the antigen binding domain of the first or second CAR is codon diverged as compared to its original sequence. In some embodiments, the codon diverged nucleotide sequence encoding the antigen binding domain of the first or second CAR is given by SEQ ID NO: 311 and the original nucleotide sequence encoding the same antigen binding domain is given by SEQ ID NO:264. In some embodiments, the antigen binding domain of the first or second CAR is given by SEQ ID NO:8. In some embodiments, the antigen binding domain of the other of the first or second CAR is not codon diverged as compared to its original sequence. In some embodiments, the nucleotide sequence encoding the antigen binding domain of the other of the first or second CAR is given by SEQ ID NO:310. In some embodiments, the antigen binding domain of the other of the first or second CAR is given by SEQ ID NO:241.

In some embodiments, the nucleotide sequence encoding the anti-GPRC5D CAR, or components thereof, such as components including a GPRC5D-binding scFv, a spacer, a transmembrane domain, and an intracellular signaling region, is codon diverged. Such codon diverged polynucleotides can be utilized in constructs for generation of engineered cells that express the encoded GPRC5D-binding and BCMA-binding cell surface protein. Thus, also provided are cells expressing the recombinant receptors encoded by the codon diverged polynucleotides provided herein and uses thereof in adoptive cell therapy, such as treatment of diseases and disorders associated with GPRC5D and/or BCMA expression, e.g., multiple myeloma.

b. Multicistronic Elements

In any of the provided embodiments, the polynucleotide further contains an internal ribosome entry site (IRES) between the first and second nucleic acid sequences to yield translation products of the first and second nucleic acid sequences after translation. For example, in some embodiments, transcription units can be engineered as a bicistronic unit containing an IRES (internal ribosome entry site), which allows coexpression of gene products (e.g., encoding a first and second chimeric receptor) by a message from a single promoter. For example, in some embodiments, the vector or construct can contain a nucleic acid encoding an anti-GPRC5D receptor (e.g., an anti-GPRC5D CAR) provided herein and a nucleic acid encoding an anti-BCMA receptor (e.g., an anti-BCMA CAR), separated by an IRES, under the regulation of a single promoter.

Alternatively, in any of the provided embodiments, the polynucleotide contains a nucleic acid sequence encoding a linking peptide between the first and second nucleic acid sequences, wherein the linking peptide separates the translation products of the first and second nucleic acid sequences during or after translation. In some aspects, the linking peptide contains a self-cleaving peptide, or a peptide that causes ribosome skipping, optionally a T2A peptide. In some embodiments, a single promoter may direct expression of an RNA that contains, in a single open reading frame (ORF), two or three genes (e.g. encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A, e.g., SEQ ID NO: 42 or 43), equine rhinitis A virus (E2A, e.g., SEQ ID NO: 40 or 41), Thosea asigna virus (T2A, e.g., SEQ ID NO: 35, 36, or 37), and porcine teschovirus-1 (P2A, e.g., SEQ ID NO: 38 or 39) as described in U.S. Patent Publication No. 20070116690.

In any of the provided polynucleotide constructs, a first nucleic acid sequence encoding a first CAR and a second nucleic acid sequencing encoding a second CAR are separated by a nucleic acid sequencing encoding a ribosomal skip element, such as a T2A. Thus, either during or after translation, the first chimeric antigen receptor and the second chimeric antigen receptor are cleaved into separate proteins. In any of the provided polynucleotide constructs, the nucleotide sequence encoding the T2A may be codon diverged.

2. Features of Polynucleotides

a. Codon Optimization

In some embodiments the polynucleotides are modified by optimization of the codons for expression in humans. In some aspects, codon optimization can be considered before and/or after the steps for splice site identification and/or splice site elimination, and/or at each of the iterative steps for reducing RNA heterogeneity. Codon optimization generally involves balancing the percentages of codons selected with the abundance, e.g., published abundance, of human transfer RNAs, for example, so that none is overloaded or limiting. In some cases, such balancing is necessary or useful because most amino acids are encoded by more than one codon, and codon usage generally varies from organism to organism. Differences in codon usage between transfected or transduced genes or nucleic acids and host cells can have effects on protein expression from the nucleic acid molecule. Table 2 below sets forth an exemplary human codon usage frequency table. In some embodiments, to generate codon-optimized nucleic acid sequences, codons are chosen to select for those codons that are in balance with human usage frequency. The redundancy of the codons for amino acids is such that different codons code for one amino acid, such as depicted in Table 2. In selecting a codon for replacement, it is desired that the resulting mutation is a silent mutation such that the codon change does not affect the amino acid sequence. Generally, the last nucleotide of the codon (e.g., at the third position) can remain unchanged without affecting the amino acid sequence.

TABLE 2
Human Codon Usage Frequency

| | ami- | | | | ami- | | |
|---|---|---|---|---|---|---|---|
| Human | no | freq./ | | Human | no | freq./ | |
| codon | acid | 1000 | number | codon | acid | 1000 | number |
| TTT | F | 17.6 | 714298 | TCT | S | 15.2 | 618711 |
| TTC | F | 20.3 | 824692 | TCC | S | 17.7 | 718892 |
| TTA | L | 7.7 | 311881 | TCA | S | 12.2 | 496448 |
| TTG | L | 12.9 | 525688 | TCG | S | 4.4 | 179419 |
| CTT | L | 13.2 | 536515 | CCT | P | 17.5 | 713233 |
| CTC | L | 19.6 | 796638 | CCC | P | 19.8 | 804620 |
| CTA | L | 7.2 | 290751 | CCA | P | 16.9 | 688038 |
| CTG | L | 39.6 | 1611801 | CCG | P | 6.9 | 281570 |
| ATT | I | 16 | 650473 | ACT | T | 13.1 | 533609 |
| ATC | I | 20.8 | 846466 | ACC | T | 18.9 | 768147 |
| ATA | I | 7.5 | 304565 | ACA | T | 15.1 | 614523 |
| ATG | M | 22 | 896005 | ACG | T | 6.1 | 246105 |
| GTT | V | 11 | 448607 | GCT | A | 18.4 | 750096 |
| GTC | V | 14.5 | 588138 | GCC | A | 27.7 | 1127679 |
| GTA | V | 7.1 | 287712 | GCA | A | 15.8 | 643471 |
| GTG | V | 28.1 | 1143534 | GCG | A | 7.4 | 299495 |
| TAT | Y | 12.2 | 495699 | TGT | C | 10.6 | 430311 |
| TAC | Y | 15.3 | 622407 | TGC | C | 12.6 | 513028 |
| TAA | * | 1 | 40285 | TGA | * | 1.6 | 63237 |
| TAG | * | 0.8 | 32109 | TGG | W | 13.2 | 535595 |
| CAT | H | 10.9 | 441711 | CGT | R | 4.5 | 184609 |
| CAC | H | 15.1 | 613713 | CGC | R | 10.4 | 423516 |
| CAA | Q | 12.3 | 501911 | CGA | R | 6.2 | 250760 |
| CAG | Q | 34.2 | 1391973 | CGG | R | 11.4 | 464485 |
| AAT | N | 17 | 689701 | AGT | S | 12.1 | 493429 |
| AAC | N | 19.1 | 776603 | AGC | S | 19.5 | 791383 |
| AAA | K | 24.4 | 993621 | AGA | R | 12.2 | 494682 |
| AAG | K | 31.9 | 1295568 | AGG | R | 12 | 486463 |
| GAT | D | 21.8 | 885429 | GGT | G | 10.8 | 437126 |
| GAC | D | 25.1 | 1020595 | GGC | G | 22.2 | 903565 |
| GAA | E | 29 | 1177632 | GGA | G | 16.5 | 669873 |
| GAG | E | 39.6 | 1609975 | GGG | G | 16.5 | 669768 |

For example, the codons TCT, TCC, TCA, TCG, AGT and AGC all code for Serine (note that T in the DNA equivalent to the U in RNA). From a human codon usage frequency, such as set forth in Table 2 above, the corresponding usage frequencies for these codons are 15.2, 17.7, 12.2, 4.4, 12.1, and 19.5, respectively. Since TCG corresponds to 4.4%, if this codon were commonly used in a gene synthesis, the tRNA for this codon would be limiting. In codon optimization, the goal is to balance the usage of each codon with the normal frequency of usage in the species of animal in which the transgene is intended to be expressed.

b. Splice Sites

Provided herein are polynucleotides in which one or more potential splice donor and/or splice acceptor sites have been identified and the nucleic acid sequence at or near the one or more of the identified splice donor sites has been modified. In some embodiments, the resulting modified nucleic acid sequence(s) is/are then synthesized and used to transduce cells to test for splicing as indicated by RNA heterogeneity.

Also provided here are polynucleotides, such as those encoding any of the antibodies, receptors (such as antigen receptors such as chimeric antigen receptors) and/or GPRC5D-specific and/or BCMA-specific binding proteins provided herein, that are or have been modified to reduce heterogeneity or contain one or more nucleic acid sequences observed herein (such as by the optimization methods) to result in improved features of the polypeptides, such as the CARs, as compared to those containing distinct, reference, sequences or that have not been modified. Among such features include improvements in RNA heterogeneity, such as that resulting from the presence of one or more splice sites, such as one or more cryptic splice sites, and/or improved expression and/or surface expression of the encoded protein, such as increased levels, uniformity, or consistency of expression among cells or different therapeutic cell compositions engineered to express the polypeptides.

Splice sites may be identified in polynucleotide sequences by harvesting RNA from the expressing cells, amplifying by reverse transcriptase polymerase chain reaction (RT-PCR) and resolving by agarose gel electrophoresis to determine the heterogeneity of the RNA, compared to the starting sequence. In some cases, improved sequences can be resubmitted to the gene synthesis vendor for further codon optimization and splice site removal, followed by further cryptic splice site evaluation, modification, synthesis and testing, until the RNA on the agarose gel exhibits minimal RNA heterogeneity.

Also provided are polynucleotides that have been modified to eliminate splice sites, such as cryptic splice sites. Genomic nucleic acid sequences generally, in nature, in a mammalian cell, undergo processing co-transcriptionally or immediately following transcription, wherein a nascent precursor messenger ribonucleic acid (pre-mRNA), transcribed from a genomic deoxyribonucleic acid (DNA) sequence, is in some cases edited by way of splicing, to remove introns, followed by ligation of the exons in eukaryotic cells. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products upon expression in eukaryotic cells.

Polynucleotides generated for the expression of transgenes are typically constructed from nucleic acid sequences, such as complementary DNA (cDNA), or portions thereof, that do not contain introns. Thus, splicing of such sequences is not expected to occur. However, the presence of cryptic splice sites within the cDNA sequence can lead to unintended or undesired splicing reactions and heterogeneity in the transcribed mRNA. Such heterogeneity results in translation of unintended protein products, such as truncated protein products with variable amino acid sequences that exhibit modified expression and/or activity.

In some embodiments, eliminating splice sites, such as cryptic splice sites, can improve or optimize expression of a transgene product, such as a polypeptide translated from the transgene, such as an anti-GPRC5D CAR polypeptide. Splicing at cryptic splice sites of an encoded transgene, such as an encoded GPRC5D CAR molecule, can lead to reduced protein expression, e.g., expression on cell surfaces, and/or reduced function, e.g., reduced intracellular signaling. Provided herein are polynucleotides, encoding anti-GPRC5D CAR proteins that have been optimized to reduce or eliminate cryptic splice sites. Also provided herein are polynucleotides encoding anti-GPRC5D CAR proteins that have been optimized for codon expression and/or in which one or more sequence, such as one identified by the methods or observations herein regarding splice sites, is present, and/or in which an identified splice site, such as any of the identified splice sites herein, is not present. Among the provided polynucleotides are those exhibiting below a certain degree of RNA heterogeneity or splice forms when expressed under certain conditions and/or introduced into a specified cell type, such as a human T cell, such as a primary human T cell, and cells and compositions and articles of manufacture containing such polypeptides and/or exhibiting such properties. In some embodiments, the RNA heterogeneity of transcribed RNA is reduced by greater than or greater than about 10%, 15%, 20%, 25%, 30%, 40%, 50% or more compared to a polynucleotide that has not been modified to remove cryptic splice sites and/or by codon optimization. In some embodiments, the provided polynucleotides encoding an anti-GPRC5D CAR exhibit RNA homogeneity of transcribed RNA that is at least 70%, 75%, 80%, 85%, 90%, or 95% or greater.

In some embodiments, eliminating splice sites, such as cryptic splice sites, can improve or optimize expression of a transgene product, such as a polypeptide translated from the transgene, such as an anti-BCMA CAR polypeptide. Splicing at cryptic splice sites of an encoded transgene, such as an encoded BCMA CAR molecule, can lead to reduced protein expression, e.g., expression on cell surfaces, and/or reduced function, e.g., reduced intracellular signaling. Provided herein are polynucleotides, encoding anti-BCMA CAR proteins that have been optimized to reduce or eliminate cryptic splice sites. Also provided herein are polynucleotides encoding anti-BCMA CAR proteins that have been optimized for codon expression and/or in which one or more sequence, such as one identified by the methods or observations herein regarding splice sites, is present, and/or in which an identified splice site, such as any of the identified splice sites herein, is not present. Among the provided polynucleotides are those exhibiting below a certain degree of RNA heterogeneity or splice forms when expressed under certain conditions and/or introduced into a specified cell type, such as a human T cell, such as a primary human T cell, and cells and compositions and articles of manufacture containing such polypeptides and/or exhibiting such properties. In some embodiments, the RNA heterogeneity of transcribed RNA is reduced by greater than or greater than about 10%, 15%, 20%, 25%, 30%, 40%, 50% or more compared to a polynucleotide that has not been modified to remove cryptic splice sites and/or by codon optimization. In some embodiments, the provided polynucleotides encoding an anti-BCMA CAR exhibit RNA homogeneity of transcribed RNA that is at least 70%, 75%, 80%, 85%, 90%, or 95% or greater.

RNA heterogeneity can be determined by any of a number of methods provided herein or described or known. In some embodiments, RNA heterogeneity of a transcribed nucleic acid is determined by amplifying the transcribed nucleic acid, such as by reverse transcriptase polymerase chain reaction (RT-PCR) followed by detecting one or more differences, such as differences in size, in the one or more amplified products. In some embodiments, the RNA heterogeneity is determined based on the number of differently sized amplified products, or the proportion of various differently sized amplified products. In some embodiments, RNA, such as total RNA or cytoplasmic polyadenylated RNA, is harvested from cells, expressing the transgene to be optimized, and amplified by reverse transcriptase polymerase chain reaction (RT-PCR) using a primer specific to the 5′ untranslated region (5′ UTR), in some cases corresponding to a portion of the promoter sequence in the expression vector, located upstream of the transgene in the transcribed RNA, and a primer specific to the 3′ untranslated region (3′ UTR), located downstream of the expressed transgene in the transcribed RNA sequence or a primer specific to a sequence within the transgene. In particular embodiments, at least one primer complementary to a sequence in the 5′ untranslated region (UTR) and at least one primer complementary to a sequence in the 3′ untranslated region (UTR) are employed to amplify the transgene. The skilled artisan can resolve RNA, such as messenger RNA, and analyze the heterogeneity thereof by several methods. Non-limiting, exemplary methods include agarose gel electrophoresis, chip-based capillary electrophoresis, analytical centrifugation, field flow fractionation, and chromatography, such as size exclusion chromatography or liquid chromatography.

In some aspects, the presence of potential cryptic splice sites (splice donor and/or acceptor sites that are present in a transcript, such as a transgene transcript, can result in RNA heterogeneity of the transcript following expression in a cell. In some embodiments, the one or more potential splice sites that can be present in the transgene transcript, that are not desired and/or that may be created in a transgene transcript from various underlying sequences are identified, following codon optimization of a transcript and/or by mutation or mistake or error in transcription. In some aspects of the provided embodiments, the splice donor sites and splice acceptor sites are identified independently. In some embodiments, the splice acceptor and/or donor site(s) is/are canonical, non-canonical, and/or cryptic splice acceptor and/or donor site(s).

In some embodiments, one or more potential splice site (e.g., canonical, non-canonical, and/or cryptic splice acceptor and/or donor site(s) or branch sites) in a polynucleotide, such as a polynucleotide encoding a transgene, such as a recombinant receptor, that may exhibit RNA heterogeneity, are identified and/or modified. Also provided are polypeptides having reduced numbers of such splice sites as compared to such reference polynucleotides.

In some aspects, identification of the one or more splice sites in a nucleic acid sequence is an iterative process. In some embodiments, splice sites can be identified using a splice site and/or codon optimization prediction tool, such as by submitting the starting or reference sequence encoding the transgene, such as a GPRC5D- or BCMA-binding receptor, e.g., anti-GPRC5D or anti-BCMA CAR, to a database, a gene synthesis vendor or other source able to computationally or algorithmically compare the starting or reference sequence to identify or predict splice sites and/or for codon optimization and/or splice site removal. In some embodiments, after modifying the sequence for codon optimization and/or splice site removal, one or more further assessment of a sequence, such as a revised or modified nucleic acid sequence, is carried out to further evaluate for splice site removal, such as cryptic splice sites, using one or more other or additional splice site prediction tool(s).

In some aspects, RNA heterogeneity can be a result of the activity of the spliceosome present in a eukaryotic cell. In some aspects, splicing is typically carried out in a series of reactions catalyzed by the spliceosome. Consensus sequences for splice sites are known, but in some aspects, specific nucleotide information defining a splice site may be complex and may not be readily apparent based on available methods. Cryptic splice sites are splice sites that are not predicted based on the standard consensus sequences and are variably activated. Hence, variable splicing of pre-mRNA at cryptic splice sites leads to heterogeneity in the transcribed mRNA products following expression in eukaryotic cells. In some cases, within spliceosomal introns, a donor site (usually at the 5′ end of the intron), a branch site (near the 3′ end of the intron) and an acceptor site (3′ end of the intron) are required for a splicing event. The splice donor site can include a GU sequence at the 5′ end of the intron, with a large less highly conserved region. The splice acceptor site at the 3′ end of the intron can terminate with an AG sequence.

In some embodiments, splice sites, including potential cryptic splice sites can be identified by comparing sequences to known splice site sequences, such as those in a sequence database. In some embodiments, splice sites can be identified by computationally by submitting nucleotide sequences for analysis by splice site prediction tools, such as Human Splice Finder (Desmet et al., Nucl. Acids Res. 37(9):e67 (2009)), a neural network splice site prediction tool, NNSplice (Reese et al., J. Comput. Biol., 4(4):311 (1997)), GeneSplicer (Pertea et al., Nucleic Acids Res. 2001 29(5): 1185-1190) or NetUTR (Eden and Brunak, Nucleic Acids Res. 32(3):1131 (2004)), which identify potential splice sites and the probability of a splicing event at such sites. Additional splice prediction tools include RegRNA, ESEfinder, and MIT splice predictor. Splice site prediction tools such as GeneSplicer has been trained and/or tested successfully on databases for different species, such as human, Drosophila melanogaster, Plasmodium falciparum, Arabidopsis thaliana, and rice. In some embodiments, different prediction tools may be adapted for different extents on different database and/or for different species. In some embodiments, the one or more prediction tools are selected based upon their utility in certain database and/or for certain species. See, e.g., Saxonov et al., (2000) Nucleic Acids Res., 28, 185-190.

In some embodiments, one or more splice site prediction tools are used to determine potential splice donor and/or acceptor sites. In some embodiments, splice site prediction tools that can be run locally; that can be retrained with a set of data at the user site; that can use databases for particular species (such as human), that can be compiled for multiple platforms, that allow real-time predictions for sequence selections, and/or that is an OSI certified open source software such that particular tools or plugins can be modified, can be employed. Exemplary tools that can be employed include NNSplice, GeneSplicer or both.

In some aspects, the splice site prediction tools can be used to identify a list of potential splice donor and/or splice acceptor sites in a sequence such as a polynucleotide sequence containing transgene sequences. In some aspects, the prediction tools also can generate one or more prediction scores for one or more sequences in the polynucleotide, that can indicate the likelihoods of the one or more sequences being a splice donor or acceptor site sequence.

In some embodiments, the prediction score for a particular splice site is compared with a threshold score or reference score to determine or identify a particular splice sites that are candidate for elimination or removal. For example, in some embodiments, the predicted splice site is identified as a potential splice site when the prediction score is greater or no less than the threshold score or reference score. In some aspects, considerations for eliminating or removing a particular splice site include the prediction score as compared to a reference score or a threshold score; and whether a particular splice site is desired or intentional (for example, when the splicing event is more advantageous or is required for regulation of transcription and/or translation). In some aspects, the likelihood that the resulting splice variant loses the desired function or has compromised function can also be considered when determining particular donor and/or acceptor sites for elimination or removal. In some aspects, the one or more potential splice donor and/or splice acceptor sites exhibit a score about or at least about 0.7, 0.75, 0.8, 0.85, 0.9, 0.95, or 1.0 (e.g., on a scale with a maximum of 1.0) of a splice event or probability of a splice event, and the site can be a candidate for splice site elimination or removal. In some aspects, the score, e.g., used by GeneSplicer, at the one or more potential splice donor and/or splice site is based on the difference between the log-odds score returned for that sequence by the true Markov model and the score is computed by the false Markov model. In particular embodiments, the splice donor sites and splice acceptor sites are evaluated independently, or individually. In some embodiments, splice donor sites and splice acceptor sites are evaluated as a splice donor/acceptor pair.

In some embodiments, one or more splice donor and/or splice acceptor site(s), such as the potential splice donor and/or acceptor sites that may be involved in a cryptic splicing event that is not desired or that results in undesired RNA heterogeneity, is eliminated. In some embodiments, eliminating one or more splice sites comprises modifying one or more nucleotides (e.g., by substitution or replacement) in, at, containing or near the splice donor and/or acceptor sites that are candidates for removal. In some aspects, a particular nucleotide within a codon that is at, contains or is near the splice site is modified (e.g., substituted or replaced). In some aspects, the modification (such as substitution or replacement) retains or preserves the amino acid encoded by the particular codon at the site, at the same time removing the potential splice donor and/or acceptor sites.

In some embodiments, the codon at or near the splice site for modification comprises one or more codons that involve one or both of the two nucleotides at the potential splice site (in some cases referred to as “splice site codon”). When the potential splicing is predicted to occur between two nucleotides in a codon, the codon is the only splice site codon for this splice site. If the potential splicing is predicted to occur between two adjacent codons, for example, between the last nucleotide of the first codon and the first nucleotide of the next codon, the two codons are splice site codons. For example, for splice sites that are predicted to be at boundaries of two codons, the two adjacent codons can be candidates for nucleotide modification. In some embodiments, the one or more codons comprise one splice site codon. In some embodiments, the one or more codons comprise both splice site codons. In some embodiments, a potential splice donor site is eliminated by modifying one or both splice site codons. In some embodiments, a potential splice acceptor donor site is eliminated by modifying one or both splice site codons. In some embodiments, the one or both codons at the splice site is not modified, for example, when there are no synonymous codon for the splice site codon. In some embodiments, if there are no synonymous codons available for the particular splice site codon, one or more nucleotides in a nearby codon can be modified. In some embodiments, one or more codons that are modified include a splice site codon, wherein the modification comprises changing one or both nucleotides at the splice site to a different nucleotide or different nucleotides. In some embodiments, In some embodiments, the splice donor site is eliminated by modifying one or both splice site codons, wherein the modification does not change one or two of the nucleotides of the at the splice site to a different nucleotide, but a nearby nucleotide, e.g., a part of a codon adjacent to the splice site, is modified. In some embodiments, the nearby or adjacent nucleotides that can be modified include modification of a nucleotide that is a part of a nearby or adjacent codon, such as a codon that is within one, two, three, four, five, six, seven, eight, nine or ten codons upstream or downstream of the splice site codon.

In some cases, polynucleotides can be manually modified, while preserving the encoded amino acid sequence, to reduce the probability of a predicted splice site. In some embodiments, one or more of the predicted splice sites having at least 80%, 85%, 90%, or 95% probability of a splice site are manually modified to reduce the probability of the splicing event. In some embodiments, the one or more modification(s) is/are by nucleotide replacement or substitution of 1, 2, 3, 4, 5, 6 or 7 nucleotides. In some embodiments, the modification(s) is/are at the junction of the splice donor site or are at the junction of the splice acceptor site. In some embodiments, at least one of the one or more nucleotide modifications is within 1, 2, 3, 4, 5, 6, 7, 8, 9 or 10 residues of the splice site junction of the splice acceptor and/or splice donor site. In some embodiments, libraries of modified nucleic acid sequences can be generated with reduced probability of cryptic splice sites. In some embodiments, splice donor sites and splice acceptor sites are evaluated as a splice donor/acceptor pair. In particular embodiments, the splice donor sites and splice acceptor sites are evaluated independently, or individually, and not part as a splice donor/acceptor pair. In some embodiments, one or more predicted splice sites are not eliminated. In some embodiments, splice sites, such as known or predicted splice sites, within the promoter region of the transcript are not eliminated.

In some embodiments, one or more potential donor splice site is eliminated by modifying one or two splice site codons or one or more nearby or adjacent codons (for example, if a synonymous codon is not available for the splice site codon). In some embodiments, one or more potential acceptor splice site is eliminated by modifying one or two splice site codons or one or more nearby or adjacent codons (for example, if a synonymous codon is not available for the splice site codon). In some embodiments, the nearby or adjacent codon that is subject to modification include a codon that is within one, two, three, four, five, six, seven, eight, nine or ten codons upstream or downstream of the splice site codon, such as a codon that is within one, two or three codons from the splice site. In some embodiments, a potential branch site for splicing is removed or eliminated. In some aspects, a nucleotide within the codon at or near the branch site can be modified, e.g., substituted or replaced, to eliminate cryptic splicing and/or reduce RNA heterogeneity. In some embodiments, the modification of the one or more nucleotides can involve a substitution or replacement of one of the nucleotides that may be involved in splicing (such as at the splice donor site, splice acceptor site or splice branch site), such that the amino acid encoded by the codon is preserved, and the nucleotide substitution or replacement does not change the polypeptide sequence that is encoded by the polynucleotide. In some cases, the third position in the codon is more degenerate than the other two positions. Thus, various synonymous codons can encode a particular amino acid (see, e.g., Section I.B.2.a. above). In some embodiments, the modification includes replacing the codon with a synonymous codon used in the species of the cell into which the polynucleotide is introduced (e.g., human). In some embodiments, the species is human. In some embodiments, the one or more codon is replaced with a corresponding synonymous codons that the most frequently used in the species or synonymous codons that have a similar frequency of usage (e.g., most closest frequency of usage) as the corresponding codon (see, e.g., Section I.B.2.a. above).

In some embodiments, the transgene candidacy for the removal of splice sites is assessed, after initial proposed modification. In some aspects, the proposed modification can be evaluated again, to assess the proposed modification and identify any further potential splice sites after modification and/or codon optimization. In some aspects, after modifying the sequence for codon optimization and/or splice site removal, one or more further assessment of a sequence, such as a revised or modified nucleic acid sequence, is carried out to further evaluate for splice site removal, such as cryptic splice sites, using the same or one or more other or additional splice site prediction tool(s). In some aspects, proposed modifications are considered for subsequent steps, and iterative optimization can be used. In some aspects, the methods any of the identification and/or modification steps may be repeated, for example, until heterogeneity of the transcript is reduced compared to the heterogeneity of the transcript as initially determined. In some embodiments, a further or a different modification, such as with a different nucleotide replacement at the same codon or a modification at a different position or codon, can be done after an iterative evaluation and assessment. In some embodiments, corresponding different synonymous codon can be used, such as the second most frequently used in the particular species or a codon that has a similar frequency of usage (e.g., the next closest frequency of usage) as the corresponding codon (see, e.g., Section II.B.2 below).

In some aspects, a proposed modification can be further evaluated, for example, to assess whether the modification generates an undesired or additional restriction site in the polynucleotide. In some aspects, an additional restriction site may not be desired, and a further or a different modification (e.g., with a different nucleotide replacement at the same codon or a modification at a different position or codon) can be considered. In some aspects, particular restriction site, such as a designated restriction site, is avoided. In some aspects, if the modification does not substantially reduce the splice site prediction score, an additional or alternative modification can be proposed. In some embodiments, the splice site prediction score can be is reduced or lowered by at least about 5%, 10%, 15%, 20%, 25%, 30%, 35%, 40%, 45%, 50%, 55%, 60%, 65%, 70% or 75%, after one or more iteration of the methods.

In some embodiments, a computer system can be used to execute one or more steps, tools, functions, processes or scripts. In some embodiments, the splice site prediction, evaluation and modification for elimination or removal of a splice site can be performed by computer implemented methods and/or by methods which include steps that are computer implemented steps. In some embodiments, comparison of the sequences to a known database, calculating a splice site prediction score, determining potential nucleotide modifications, codon optimization and/or any one of the iterative steps can be implemented by a computer or using a computer-implemented steps, tools, functions, processes or scripts. In particular embodiments, a computer system comprising a processor and memory is provided, wherein the memory contains instructions operable to cause the processor to carry out any one or more of steps of the methods provided herein. In some embodiments, steps, functions, processes or scripts are performed computationally, e.g., performed using one or more computer programs and/or via the use of computational algorithms.

Exemplary steps, functions, processes or scripts for identifying and/or removing possible splice sites include one or more steps of: selecting sequence, writing FASTA format sequences, loading codon table (e.g., from www.kazusa.or.jp/codon, running GeneSplicer, loading predictions, parsing codons, determining overlaps in prediction, identifying next highest usage synonymous codon, reviewing for restriction site, creating annotations or assessing other codons. Particular steps can assess both forward and reverse strands. In some aspects, previously annotated splice site modifications can also be considered, to allow for iterative optimization. In some embodiments, any one or more of the steps, functions, processes or scripts can be repeated.

In some embodiments, a provided polynucleotide encoding an anti-GPRC5D CAR provided herein, or a construct provided herein, includes modifications to remove one or more splice donor and/or acceptor site that may contribute to splice events and/or reduced expression and/or increased RNA heterogeneity. In some embodiments, provided polynucleotides are modified in one or more polynucleotides in the spacer region to eliminate or reduce splice events. Among potential splice donor and/or acceptor sites that are modified or not included in a provided CAR are set forth in SEQ ID NO: 176, 177, 178, 179, 180 or 181. In some embodiments, modified nucleotides of such sites to reduce or eliminate potential splice and/or donor sites are set forth in SEQ ID NO: 182, 183, 184, 185, 186, 187 or 188. In some embodiments, a provided polynucleotide encoding an anti-GPRC5D CAR, or other CAR, contains one or more nucleotide sequences set forth in SEQ ID NO: 182, 183, 184, 185, 186, 187 or 188. In some embodiments, a provided anti-GPRC5D CAR includes the nucleotide sequence set forth in SEQ ID NO:74. In some embodiments, the spacer is encoded by the nucleotide sequence set forth in SEQ ID NO:283. In some embodiments, the spacer is encoded by the nucleotide sequence set forth in SEQ ID NO:284. In some embodiments, the spacer is encoded by the nucleotide sequence set forth in SEQ ID NO:305.

c. Other Features

Also provided are vectors containing the polynucleotides and host cells containing the vectors, e.g., for producing the chimeric antigen receptors. Also provided are methods for producing the chimeric antigen receptors. The nucleic acid may encode a chimeric antigen receptor comprising a VL region and/or a VH region of an antibody (e.g., the light and/or heavy chains of the antibody). The nucleic acid may encode one or more amino chimeric antigen receptors each comprising a VL region and/or a VH region of an antibody (e.g., the light and/or heavy chains of the antibody). In a further embodiment, one or more vectors (e.g., expression vectors) comprising such polynucleotides are provided. In a further embodiment, a host cell comprising such polynucleotides is provided. In one such embodiment, a host cell comprises (e.g., has been transformed with) a vector comprising a nucleic acid that encodes chimeric antigen receptor comprising the VH region of an antibody. In another such embodiment, a host cell comprises (e.g., has been transformed with) (1) a vector comprising a nucleic acid that encodes a chimeric antigen receptor comprising the VL region of the antibody and the VH region of the antibody, or (2) a first vector comprising a nucleic acid that encodes a chimeric antigen receptor comprising a first antibody and a second vector comprising a nucleic acid that encodes a chimeric antigen receptor comprising a second antibody. In some embodiments, a host cell comprises (e.g., has been transformed with) one or more vectors comprising one or more nucleic acid that encodes one or more chimeric antigen receptors. In some embodiments, one or more such host cells are provided. In some embodiments, a composition containing one or more such host cells are provided. In some embodiments, the one or more host cells can express different chimeric antigen receptors, or the same chimeric antigen receptor. In some embodiments, each of the host cells can express more than one chimeric antigen receptor.

Also provided are methods of making the anti-GPRC5D chimeric antigen receptors. For recombinant production of the chimeric receptors, a nucleic acid sequence encoding a chimeric receptor antibody, e.g., as described herein, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the anti-GPRC5D chimeric antigen receptor is provided, wherein the method comprises culturing a host cell comprising a nucleic acid sequence encoding the antibody, as provided above, under conditions suitable for expression of the receptor.

Also provided are methods of making the anti-BCMA chimeric antigen receptors. For recombinant production of the chimeric receptors, a nucleic acid sequence encoding a chimeric receptor antibody, e.g., as described herein, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the anti-BCMA chimeric antigen receptor is provided, wherein the method comprises culturing a host cell comprising a nucleic acid sequence encoding the antibody, as provided above, under conditions suitable for expression of the receptor.

Also provided are methods of making chimeric antigen constructs comprising both an anti-GPRC5D chimeric antigen receptor and an anti-BCMA chimeric antigen receptor. For recombinant production of the chimeric receptors, a nucleic acid sequence encoding both of the chimeric receptor antibodies, e.g., as described herein, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the dual CARs is provided, wherein the method comprises culturing a host cell comprising a nucleic acid sequence encoding the antibodies, as provided above, under conditions suitable for expression of the receptor.

Also provided are methods of making chimeric antigen receptors that bind both GPRC5D and BCMA. For recombinant production of the chimeric receptors, a nucleic acid sequence encoding both chimeric receptor antibodies, e.g., as described herein, may be isolated and inserted into one or more vectors for further cloning and/or expression in a host cell. Such nucleic acid sequences may be readily isolated and sequenced using conventional procedures (e.g., by using oligonucleotide probes that are capable of binding specifically to genes encoding the heavy and light chains of the antibody). In some embodiments, a method of making the chimeric antigen receptor that binds BCMA and GPRC5D is provided, wherein the method comprises culturing a host cell comprising a nucleic acid sequence encoding the antibody, as provided above, under conditions suitable for expression of the receptor.

In some embodiments, a method of making a cellular composition comprising cells expressing the anti-BCMA chimeric antigen receptor and cells expressing the anti-GPRC5D chimeric antigen receptor is provided.

In addition to prokaryotes, eukaryotic microbes such as filamentous fungi or yeast are suitable cloning or expression hosts for antibody-encoding vectors, including fungi and yeast strains whose glycosylation pathways have been modified to mimic or approximate those in human cells, resulting in the production of an antibody with a partially or fully human glycosylation pattern. See Gerngross, Nat. Biotech. 22:1409-1414 (2004), and Li et al., Nat. Biotech. 24:210-215 (2006).

Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, the antibody heavy chains and/or light chains (e.g., VH region and/or VL region) may be expressed in yeast (see, e.g., U.S. Publication No. US 2006/0270045 A1). In some embodiments, a particular eukaryotic host cell is selected based on its ability to make desired post-translational modifications to the heavy chains and/or light chains (e.g., VH region and/or VL region). For example, in some embodiments, CHO cells produce polypeptides that have a higher level of sialylation than the same polypeptide produced in 293 cells. In particular examples immune cells, such as human immune cells are used to express the provided polypeptides encoding chimeric antigen receptors. In some examples, the immune cells are T cells, such as CD4+ and/or CD8+ immune cells.

### II. BCMA-Binding Receptors and Encoding Polynucleotides

Provided in some aspects are BCMA-binding agents, such as recombinant receptors or chimeric antigen receptors that bind BCMA molecules and polynucleotides encoding BCMA binding cell surface proteins, such as recombinant receptors (e.g., CARs), and cells expressing such receptors. The BCMA-binding cell surface proteins generally contain antibodies (e.g., antigen-binding antibody fragments), and/or other binding peptides that specifically bind to BCMA, such as to BCMA proteins, such as human BCMA protein. In some aspects, the agents bind to an extracellular portion of BCMA.

Among the provided polynucleotides are those that encode recombinant receptors, such as antigen receptors, that specifically bind BCMA. In some aspects, the encoded receptors, such as those containing BCMA-binding polypeptides, and compositions and articles of manufacture and uses of the same, also are provided.

Among the BCMA-binding polypeptides are antibodies, such as single-chain antibodies (e.g., antigen binding antibody fragments), or portions thereof. In some examples, the recombinant receptors are chimeric antigen receptors, such as those containing anti-BCMA antibodies or antigen-binding fragments thereof. The provided polynucleotides can be incorporated into constructs, such as deoxyribonucleic acid (DNA) or RNA constructs, such as those that can be introduced into cells for expression of the encoded recombinant BCMA-binding receptors.

The polynucleotides encoding BCMA-binding polypeptides comprise features as set forth in similar preceding sections, including Section I (e.g., Section I.C.).

The provided BCMA-binding receptors generally contain an extracellular binding molecule and an intracellular signaling domain Among the provided receptors are polypeptides containing antibodies, such as recombinant cell surface receptors containing anti-BCMA. Such receptors include chimeric antigen receptors that contain such antibodies.

Among the provided recombinant receptors are chimeric antigen receptors that include a BCMA-binding fragment. The recombinant receptors include chimeric antigen receptors that specifically bind to BCMA, such as antigen receptors containing the anti-BCMA antibodies, e.g. BCMA antigen-binding fragments. Among the antigen receptors are functional non-TCR antigen receptors, such as chimeric antigen receptors (CARs). Also provided are cells expressing the recombinant receptors and uses thereof in adoptive cell therapy, such as treatment of diseases and disorders associated with BCMA expression, e.g., multiple myeloma.

Among the chimeric receptors are chimeric antigen receptors (CARs). The chimeric receptors, such as CARs, generally include an extracellular antigen binding domain that includes, is, or comprises an anti-BCMA antibody. Thus, the chimeric receptors, e.g., CARs, typically include in their extracellular portions one or more BCMA-binding molecules, such as one or more antigen-binding fragment, domain, or portion, or one or more antibody variable regions, and/or antibody molecules, such as those described herein.

In some embodiments, the first CAR includes a GPRC5D-binding portion or portions of the antibody molecule, such as a heavy chain variable (VH) region and/or light chain variable (VL) region of the antibody, e.g., an scFv antibody fragment. In some embodiments, the provided GPRC5D-binding CARs contain an antibody, such as an anti-GPRC5D antibody, or an antigen-binding fragment thereof that confers the GPRC5D-binding properties of the provided CAR. In some embodiments, the antibody or antigen-binding domain can be any anti-GPRC5D antibody described or derived from any anti-GPRC5D antibody described (see, e.g., WO 2016/090312, WO 2016/090329, WO 2018/017786). Any of such anti-GPRC5D antibodies or antigen-binding fragments can be used in the provided CARs. In some embodiments, the anti-GPRC5D CAR contains an antigen-binding domain that is an scFv containing a variable heavy (VH) and/or a variable light (VL) region derived from an antibody described in WO 2016/090312, WO 2016/090329, or WO 2018/017786.

In some embodiments, the antibody, e.g., the anti-GPRC5D antibody, or antigen-binding fragment, contains a heavy and/or light chain variable (VH or VL) region sequence as described, or a sufficient antigen-binding portion thereof. In some embodiments, the anti-GPRC5D antibody, e.g., antigen-binding fragment, contains a VH region sequence or sufficient antigen-binding portion thereof that contains a CDR-H1, CDR-H2 and/or CDR-H3 as described. In some embodiments, the anti-GPRC5D antibody, e.g., antigen-binding fragment, contains a VL region sequence or sufficient antigen-binding portion that contains a CDR-L1, CDR-L2 and/or CDR-L3 as described. In some embodiments, the anti-GPRC5D antibody, e.g., antigen-binding fragment, contains a VH region sequence that contains a CDR-H1, CDR-H2 and/or CDR-H3 as described and contains a VL region sequence that contains a CDR-L1, CDR-L2 and/or CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence.

In some embodiments, the antibody or antibody fragment, in the provided CAR, has a VH region of any of the antibodies or antibody binding fragments described in any of WO 2016/090312, WO 2016/090329, and WO 2018/017786.

In some embodiments, the CAR contains an antibody or antigen-binding fragment thereof, that has a heavy chain variable (VH) region having the amino acid sequence selected from any one of SEQ ID NOs: 21, 23, 25, 27, 29, 31, or 33, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VH region amino acid selected from any one of SEQ ID NOs: 21, 23, 25, 27, 29, 31, or 33, or contains a CDR-H1, CDR-H2, and/or CDR-H3 present in such a VH sequence.

In some embodiments, the VH region of an antibody or antigen-binding fragment thereof comprises a CDR-H1, CDR-H2, and/or CDR-H3 according to Kabat numbering. In some embodiments, the VH region of an antibody or antigen-binding fragment thereof comprises a CDR-H1, CDR-H2, and/or CDR-H3 according to Chothia numbering. In some embodiments, the VH region of an antibody or antigen-binding fragment thereof comprises a CDR-H1, CDR-H2, and/or CDR-H3 according to AbM numbering.

In some embodiments, the CAR contains an antibody or antigen-binding fragment thereof, that has a variable heavy chain (VH) region comprising a CDR-H1 comprising the amino acid sequence selected from SEQ ID NOs: 75, 78, 80, 82, 90, 93, 95, 97, 105, 108, 110, 112, 120, 123, 125, 127, 135, 138, 140, 142, 152, 162, 165, 167, and 169; (b) a CDR-H2 comprising the amino acid sequence selected from SEQ ID NOs: 76, 79, 81, 83, 91, 94, 96, 98, 106, 109, 111, 113, 121, 124, 126, 128, 136, 139, 141, 143, 150, 153, 154, 155, 163, 166, 168, and 170; and (c) a CDR-H3 comprising the amino acid sequence selected from SEQ ID NOs: 77, 84, 92, 99, 107, 114, 122, 129, 137, 144, 151, 156, 164, and 171.

In some embodiments, the antibody or antigen-binding fragment thereof comprises a VH region comprising a CDR-H1, CDR-H2, and CDR-H3 comprising the amino acid sequence of SEQ ID NOs:75, 76 and 77, respectively; SEQ ID NOs:78, 79 and 77, respectively; SEQ ID NOs:80, 81 and 77, respectively; SEQ ID NOs:82, 83 and 84, respectively; SEQ ID NOs:90, 91 and 92, respectively; SEQ ID NOs:93, 94 and 92, respectively; SEQ ID NOs:95, 96 and 92, respectively; SEQ ID NOs:97, 98 and 99, respectively; SEQ ID NOs:105, 106 and 107, respectively; SEQ ID NOs:108, 109 and 107, respectively; SEQ ID NOs:110, 111 and 107, respectively; SEQ ID NOs:112, 113 and 114, respectively; SEQ ID NOs:120, 121 and 122, respectively; SEQ ID NOs:123, 124 and 122, respectively; SEQ ID NOs:125, 126 and 122, respectively; SEQ ID NOs:127, 128 and 129, respectively; SEQ ID NOs:135, 136 and 137, respectively; SEQ ID NOs:138, 139 and 137, respectively; SEQ ID NOs:140, 141 and 137, respectively; SEQ ID NOs:142, 143 and 144, respectively; SEQ ID NOs:135, 150 and 151, respectively; SEQ ID NOs:152, 153 and 151, respectively; SEQ ID NOs:140, 154 and 151, respectively; SEQ ID NOs:142, 155 and 156, respectively; SEQ ID NOs:162, 163 and 164, respectively; SEQ ID NOs:165, 166 and 164, respectively; SEQ ID NOs:167, 168 and 164, respectively; SEQ ID NOs:169, 170 and 171, respectively.

In some embodiments, the antibody or antigen-binding fragment thereof comprises a VH region comprising the amino acid sequence of SEQ ID NOs:75, 76 and 77, respectively; SEQ ID NOs:78, 79 and 77, respectively; SEQ ID NOs:80, 81 and 77, respectively; SEQ ID NOs:82, 83 and 84, respectively; SEQ ID NOs:90, 91 and 92, respectively; SEQ ID NOs:93, 94 and 92, respectively; SEQ ID NOs:95, 96 and 92, respectively; SEQ ID NOs:97, 98 and 99, respectively; SEQ ID NOs:105, 106 and 107, respectively; SEQ ID NOs:108, 109 and 107, respectively; SEQ ID NOs:110, 111 and 107, respectively; SEQ ID NOs:112, 113 and 114, respectively; SEQ ID NOs:120, 121 and 122, respectively; SEQ ID NOs:123, 124 and 122, respectively; SEQ ID NOs:125, 126 and 122, respectively; SEQ ID NOs:127, 128 and 129, respectively; SEQ ID NOs:135, 136 and 137, respectively; SEQ ID NOs:138, 139 and 137, respectively; SEQ ID NOs:140, 141 and 137, respectively; SEQ ID NOs:142, 143 and 144, respectively; SEQ ID NOs:135, 150 and 151, respectively; SEQ ID NOs:152, 153 and 151, respectively; SEQ ID NOs:140, 154 and 151, respectively; SEQ ID NOs:142, 155 and 156, respectively; SEQ ID NOs:162, 163 and 164, respectively; SEQ ID NOs:165, 166 and 164, respectively; SEQ ID NOs:167, 168 and 164, respectively; SEQ ID NOs:169, 170 and 171, respectively.

In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the VH region amino acid sequence set forth in any one of SEQ ID NOs: 21, 23, 25, 27, 29, 31, or 33.

In some embodiments of the antibody or antigen-binding fragment thereof provided herein, the VH region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the VH region amino acid sequence set forth in any one of SEQ ID NOs: 21, 23, 25, 27, 29, 31, or 33.

In some embodiments, the antibody or antigen-binding fragment thereof comprises a VH region comprising the amino acid sequence set forth in any one of SEQ ID NOs: 21, 23, 25, 27, 29, 31, or 33.

In some embodiments, the antibody or antibody fragment, in the provided CAR (e.g. an anti-GPRC5D CAR), comprising a VH region further comprises a light chain or a sufficient antigen binding portion thereof. For example, in some embodiments, the antibody or antigen-binding fragment thereof contains a VH region and a VL region, or a sufficient antigen-binding portion of a VH and VL region. In such embodiments, a VH region sequence can be any of the above described VH sequence. In some such embodiments, the antibody is an antigen-binding fragment, such as a Fab or an scFv. In some such embodiments, the antibody is a full-length antibody that also contains a constant region.

In some embodiments, a CAR provided herein, contains an antibody such as an anti-GPRC5D antibody, or antigen-binding fragment thereof that contains any of the above VH region and contains a variable light chain region or a sufficient antigen binding portion thereof. For example, in some embodiments, the CAR contains an antibody or antigen-binding fragment thereof that contains a VH region and a variable light chain (VL) region, or a sufficient antigen-binding portion of a VH and VL region. In such embodiments, a VH region sequence can be any of the above described VH sequence. In some such embodiments, the antibody is an antigen-binding fragment, such as a Fab or an scFv. In some such embodiments, the antibody is a full-length antibody that also contains a constant region.

In some embodiments, the antibody or antigen-binding fragment has a V_{L} region described in any of WO 2016/090312, WO 2016/090329, and WO 2018/017786.

In some embodiments, the CAR contains an antibody or antigen-binding fragment thereof, that has a light chain variable (VL) region having the amino acid sequence selected from any one of SEQ ID NOs: 22, 24, 26, 28, 30, 32, 34, 63, 64, 65, 66, 67, 68, or 69, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VL region amino acid selected from any one of SEQ ID NOs: 22, 24, 26, 28, 30, 32, 34, 63, 64, 65, 66, 67, 68, or 69, or contains a CDR-L1, CDR-L2, and/or CDR-L3 present in such a VL sequence. In some embodiments, the CAR contains an antibody or antigen-binding fragment thereof, that has a light chain variable (VL) region having the amino acid sequence selected from any one of SEQ ID NOs: 22, 24, 26, 28, 30, 32, or 34, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VL region amino acid selected from any one of SEQ ID NOs: 22, 24, 26, 28, 30, 32, or 34, or contains a CDR-L1, CDR-L2, and/or CDR-L3 present in such a VL sequence. In some embodiments, the CAR contains an antibody or antigen-binding fragment thereof, that has a light chain variable (VL) region having the amino acid sequence selected from any one of SEQ ID NOs: 63, 64, 65, 66, 67, 68 or 69, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the VL region amino acid selected from any one of SEQ ID NOs: 63, 64, 65, 66, 67, 68, or 69, or contains a CDR-L1, CDR-L2, and/or CDR-L3 present in such a VL sequence.

In some embodiments, the VL region of an antibody or antigen-binding fragment thereof comprises a CDR-L1, CDR-L2, and/or CDR-L3 according to Kabat numbering. In some embodiments, the VL region of an antibody or antigen-binding fragment thereof comprises a CDR-L1, CDR-L2, and/or CDR-L3 according to Chothia numbering. In some embodiments, the VL region of an antibody or antigen-binding fragment thereof comprises a CDR-L1, CDR-L2, and/or CDR-L3 according to AbM numbering.

In some embodiments, the CAR contains an antibody or antigen-binding fragment thereof, that has a variable light chain (VL) region comprising a CDR-L1 comprising the amino acid sequence selected from SEQ ID NOs: 85, 88, 100, 103, 115, 118, 130, 133, 145, 148, 157, 160, 172, and 174; (b) a CDR-L2 comprising the amino acid sequence selected from SEQ ID NOs: 86, 89, 101, 104, 116, 119, 131, 134, 146, 149, 158, and 161; and (c) a CDR-L3 comprising the amino acid sequence selected from SEQ ID NOs: 87, 102, 117, 132, 147, 159, 173, and 175.

In some embodiments, the antibody or antigen-binding fragment thereof comprises a VL region comprising a CDR-L1, CDR-L2, and CDR-L3 comprising the amino acid sequence of SEQ ID NOs:85, 86 and 87, respectively; SEQ ID NOs:88, 89 and 87, respectively; SEQ ID NOs:100, 101 and 102, respectively; SEQ ID NOs:103, 104 and 102, respectively; SEQ ID NOs:115, 116 and 117, respectively; SEQ ID NOs:118, 119 and 117, respectively; SEQ ID NOs:130, 131 and 132, respectively; SEQ ID NOs:133, 134 and 132, respectively; SEQ ID NOs:145, 146 and 147, respectively; SEQ ID NOs:148, 149 and 147, respectively; SEQ ID NOs:157, 158 and 159, respectively; SEQ ID NOs:160, 161 and 159, respectively; SEQ ID NOs:172, 86 and 173, respectively; SEQ ID NOs:174, 89 and 175, respectively; SEQ ID NOs:174, 89 and 297, respectively.

In some embodiments, the antibody or antigen-binding fragment thereof comprises a VL region comprising the amino acid sequence of SEQ ID NOs:85, 86 and 87, respectively; SEQ ID NOs:88, 89 and 87, respectively; SEQ ID NOs:100, 101 and 102, respectively; SEQ ID NOs:103, 104 and 102, respectively; SEQ ID NOs:115, 116 and 117, respectively; SEQ ID NOs:118, 119 and 117, respectively; SEQ ID NOs:130, 131 and 132, respectively; SEQ ID NOs:133, 134 and 132, respectively; SEQ ID NOs:145, 146 and 147, respectively; SEQ ID NOs:148, 149 and 147, respectively; SEQ ID NOs:157, 158 and 159, respectively; SEQ ID NOs:160, 161 and 159, respectively; SEQ ID NOs:172, 86 and 173, respectively; SEQ ID NOs:174, 89 and 175, respectively; SEQ ID NOs:174, 89 and 297, respectively.

In some embodiments, the antibody or antigen-binding fragment thereof contains a CDR-L1, CDR-L2, and CDR-L3, respectively, contained within the VL region amino acid sequence selected from any one of SEQ ID NOs: 22, 24, 26, 28, 30, 32, 34, 63, 64, 65, 66, 67, 68, or 69. In some embodiments, the antibody or antigen-binding fragment thereof contains a CDR-L1, CDR-L2, and CDR-L3, respectively, contained within the VL region amino acid sequence selected from any one of SEQ ID NOs: 22, 24, 26, 28, 30, 32, or 34. In some embodiments, the antibody or antigen-binding fragment thereof contains a CDR-L1, CDR-L2, and CDR-L3, respectively, contained within the VL region amino acid sequence selected from any one of SEQ ID NOs: 63, 64, 65, 66, 67, 68, or 69.

Among the CARs provided herein is a CAR in which the antibody, such as an anti-GPRC5D antibody, or antibody fragment, in the provided CAR, comprises a VH region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in any of SEQ ID NOs: 21, 23, 25, 27, 29, 31, or 33 and a VL region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in any one of SEQ ID NOs: 22, 24, 26, 28, 30, 32, 34, 63, 64, 65, 66, 67, 68, or 69.

In some embodiments, the VH region of the antibody or antigen-binding fragment thereof comprises a CDR-H1, a CDR-H2, a CDR-H3, respectively, comprising the amino acid sequences of CDR-H1, CDR-H2, and CDR-H3 contained within the VH region amino acid sequence selected from any one of SEQ ID NOs: 21, 23, 25, 27, 29, 31, or 33; and comprises a CDR-L1, a CDR-L2, a CDR-L3, respectively, comprising the amino acid sequences of CDR-L1, CDR-L2, and CDR-L3, respectively contained within the VL region amino acid sequence selected from any one of SEQ ID NOs: 22, 24, 26, 28, 30, 32, 34, 63, 64, 65, 66, 67, 68, or 69.

In some embodiments, the VH region of the antibody or antigen-binding fragment thereof comprise the amino acid sequence of SEQ ID NOs: 21, 23, 25, 27, 29, 31, or 33 and the and VL regions of the antibody or antigen-binding fragment comprises the amino acid sequence 22, 24, 26, 28, 30, 32, or 34. In some embodiments, the VH and VL regions of the antibody or antigen-binding fragment thereof comprise the amino acid sequences of SEQ ID NOs: 21 and 22, respectively; SEQ ID NOs: 23 and 24, respectively; SEQ ID NOs: 25 and 26, respectively; SEQ ID NOs: 27 and 28, respectively; SEQ ID NOs: 29 and 30, respectively; SEQ ID NOs: 31 and 32, respectively; or SEQ ID NOs: 33 and 34, respectively, or any antibody or antigen-binding fragment thereof that has at least 90% sequence identity to any of the above VH and VL, such as at least 90%, 91%, 92%, 93%, 94%, 95%, 96%, 97%, 98%, or 99% sequence identity thereto.

For example, the VH and VL regions of the antibody or antigen-binding fragment thereof provided therein comprise the amino acid sequences selected from: SEQ ID NOs: 21 and 22; SEQ ID NOs: 23 and 24; SEQ ID NOs: 25 and 26; SEQ ID NOs: 27 and 28; SEQ ID NOs: 29 and 30; SEQ ID NOs: 31 and 32; SEQ ID NOs: 33 and 34, respectively. In other examples, the VH and VL regions of the antibody or antigen-binding fragment thereof provided therein comprise the amino acid sequences selected from: SEQ ID NOs: 21 and 63; SEQ ID NOs: 23 and 64; SEQ ID NOs: 25 and 65; SEQ ID NOs: 27 and 66; SEQ ID NOs: 29 and 67; SEQ ID NOs: 31 and 68; SEQ ID NOs: 33 and 69, respectively.

In some embodiments, the antibody or antigen-binding fragment thereof, in the provided CAR, is a single-chain antibody fragment, such as a single chain variable fragment (scFv) or a diabody or a single domain antibody (sdAb). In some embodiments, the antibody or antigen-binding fragment is a single domain antibody comprising only the VH region. In some embodiments, the antibody or antigen binding fragment is an scFv comprising a heavy chain variable (VH) region and a light chain variable (VL) region. In some embodiments, the single-chain antibody fragment (e.g., scFv) includes one or more linkers joining two antibody domains or regions, such as a heavy chain variable (VH) region and a light chain variable (VL) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided CARs contain anti-GPRC5D antibodies that include single-chain antibody fragments, such as scFvs and diabodies, particularly human single-chain antibody fragments, typically comprising linker(s) joining two antibody domains or regions, such VH and VL regions. The linker typically is a peptide linker, e.g., a flexible and/or soluble peptide linker, such as one rich in glycine and serine.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 50) or GGGS (3GS; SEQ ID NO: 51), such as between 2, 3, 4, and 5 repeats of such a sequence. Exemplary linkers include those having or consisting of a sequence set forth in SEQ ID NO: 52 (GGGGSGGGGSGGGGS). Exemplary linkers further include those having or consisting of the sequence set forth in SEQ ID NO: 53 (GSTSGSGKPGSGEGSTKG). Exemplary linkers further include those having or consisting of the sequence set forth in SEQ ID NO: 54 (SRGGGGSGGGGSGGGGSLEMA). An exemplary linker includes those having or consisting of the sequence set forth in SEQ ID NO; 47 (GSRGGGGSGGGGSGGGGSLEMA).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 51) or GGGGS (SEQ ID NO: 50), such as the linker set forth in SEQ ID NO: 47, 52 or 54.

In some embodiments, the VH region may be amino terminal to the VL region. In some embodiments, the VH region may be carboxy terminal to the VL region. In particular embodiments, the fragment, e.g., scFv, may include a VH region or portion thereof, followed by the linker, followed by a VL region or portion thereof. In other embodiments, the fragment, e.g., the scFv, may include the VL region or portion thereof, followed by the linker, followed by the VH region or portion thereof.

In some aspects, an scFv provided herein comprises the amino acid sequence selected from any one of SEQ ID NOs: 1-14, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from any one of SEQ ID NOs: 1-14.

Among a provided anti-GPRC5D CAR is a CAR in which the antibody or antigen-binding fragment contains a VH region comprising the sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and contains a VL region comprising the sequence set forth in SEQ ID NO:22 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:22. In some embodiments, the provided CAR is a CAR in which the antibody or antigen-binding fragment contains a VH region comprising the sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and contains a VL region comprising the sequence set forth in SEQ ID NO:63 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:63. In some embodiments, the antibody or antigen-binding fragment of the provided CAR contains a VH region that has a CDRH1, a CDRH2 and a CDRH3 comprising the amino acid sequence of SEQ ID NOS: 75, 76 and 77, respectively and a VL region that has a CDRL1, a CDRL2 and a CDRL3 comprising the amino acid sequence of SEQ ID NOS: 85, 86, and 87, respectively. In some embodiments, the antibody or antigen-binding fragment of the provided CAR contains a VH region that has a CDRH1, a CDRH2 and a CDRH3 comprising the amino acid sequence of SEQ ID NOS: 78, 79 and 77, respectively and a VL region that contains a CDRL1, a CDRL2 and a CDRL3 comprising the amino acid sequence of SEQ ID NOS: 85, 86, and 87, respectively. In some embodiments, the antibody or antigen-binding fragment of the provided CAR contains a VH region that has a CDRH1, a CDRH2 and a CDRH3 comprising the amino acid sequence of SEQ ID NOS: 80, 81 and 77, respectively and a VL region that contains a CDRL1, a CDRL2 and a CDRL3 comprising the amino acid sequence of SEQ ID NOS: 85, 86, and 87, respectively. In some embodiments, the antibody or antigen-binding fragment of the provided CAR contains a VH region that has a CDRH1, a CDRH2 and a CDRH3 comprising the amino acid sequence of SEQ ID NOS: 82, 83 and 84, respectively and a VL region that contains a CDRL1, a CDRL2 and a CDRL3 comprising the amino acid sequence of SEQ ID NOS: 88, 89 and 87, respectively. In some embodiments, the VH region comprises the sequence set forth in SEQ ID NO:21 and the VL region comprises the sequence set forth in SEQ ID NO:22. In some embodiments, the VH region comprises the sequence set forth in SEQ ID NO:21 and the VL region comprises the sequence set forth in SEQ ID NO:63. In some embodiments, the antibody or antigen-binding fragment is a single-chain antibody fragment, such as an scFv. In some embodiments, the scFv comprises the sequence of amino acids set forth in SEQ ID NO:1 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:1. In some embodiments, the scFv is encoded by a nucleotide sequence set forth in SEQ ID NO:257 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:257. In some embodiments, the scFv comprises the sequence of amino acids set forth in SEQ ID NO:2 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:2. n some embodiments, the scFv is encoded by a nucleotide sequence set forth in SEQ ID NO:258 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:258.

Among a provided anti-GPRC5D CAR is a CAR in which the antibody or antigen-binding fragment contains a VH region comprising the sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and contains a VL region comprising the sequence set forth in SEQ ID NO:24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24. In some embodiments, the provided CAR is a CAR in which the antibody or antigen-binding fragment contains a VH region comprising the sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and contains a VL region comprising the sequence set forth in SEQ ID NO:64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:64. In some embodiments, the antibody or antigen-binding fragment of the provided CAR contains a VH region that has a CDRH1, a CDRH2 and a CDRH3 comprising the amino acid sequence of SEQ ID NOS: 90, 91, 92, respectively and a VL region that has a CDRL1, a CDRL2 and a CDRL3 comprising the amino acid sequence of SEQ ID NOS:100, 101 and 102, respectively. In some embodiments, the antibody or antigen-binding fragment of the provided CAR contains a VH region that has a CDRH1, a CDRH2 and a CDRH3 comprising the amino acid sequence of SEQ ID NOS: 93, 94 and 92, respectively and a VL region that contains a CDRL1, a CDRL2 and a CDRL3 comprising the amino acid sequence of SEQ ID NOS: 100, 101 and 102, respectively. In some embodiments, the antibody or antigen-binding fragment of the provided CAR contains a VH region that has a CDRH1, a CDRH2 and a CDRH3 comprising the amino acid sequence of SEQ ID NOS: 95, 96 and 92, respectively and a VL region that contains a CDRL1, a CDRL2 and a CDRL3 comprising the amino acid sequence of SEQ ID NOS: 100, 101 and 102, respectively. In some embodiments, the antibody or antigen-binding fragment of the provided CAR contains a VH region that has a CDRH1, a CDRH2 and a CDRH3 comprising the amino acid sequence of SEQ ID NOS: 97, 98 and 99, respectively and a VL region that contains a CDRL1, a CDRL2 and a CDRL3 comprising the amino acid sequence of SEQ ID NOS: 103, 104 and 102, respectively. In some embodiments, the VH region comprises the sequence set forth in SEQ ID NO:23 and the VL region comprises the sequence set forth in SEQ ID NO:24. In some embodiments, the VH region comprises the sequence set forth in SEQ ID NO:23 and the VL region comprises the sequence set forth in SEQ ID NO:64. In some embodiments, the antibody or antigen-binding fragment is a single-chain antibody fragment, such as an scFv. In some embodiments, the scFv comprises the sequence of amino acids set forth in SEQ ID NO:3 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:3. In some embodiments, the scFv is encoded by a nucleotide sequence set forth in SEQ ID NO:259 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:259. In some embodiments, the scFv comprises the sequence of amino acids set forth in SEQ ID NO:4 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:4. In some embodiments, the scFv is encoded by a nucleotide sequence set forth in SEQ ID NO:260 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:260.

Among a provided anti-GPRC5D CAR is a CAR in which the antibody or antigen-binding fragment contains a VH region comprising the sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25; and contains a VL region comprising the sequence set forth in SEQ ID NO:26 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:26. In some embodiments, the provided CAR is a CAR in which the antibody or antigen-binding fragment contains a VH region comprising the sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25; and contains a VL region comprising the sequence set forth in SEQ ID NO:65 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:65. In some embodiments, the antibody or antigen-binding fragment of the provided CAR contains a VH region that has a CDRH1, a CDRH2 and a CDRH3 comprising the amino acid sequence of SEQ ID NOS: 105, 106, 107, respectively and a VL region that has a CDRL1, a CDRL2 and a CDRL3 comprising the amino acid sequence of SEQ ID NOS: 115, 116 and 117, respectively. In some embodiments, the antibody or antigen-binding fragment of the provided CAR contains a VH region that has a CDRH1, a CDRH2 and a CDRH3 comprising the amino acid sequence of SEQ ID NOS: 108, 109 and 107, respectively and a VL region that contains a CDRL1, a CDRL2 and a CDRL3 comprising the amino acid sequence of SEQ ID NOS: 115, 116 and 117, respectively. In some embodiments, the antibody or antigen-binding fragment of the provided CAR contains a VH region that has a CDRH1, a CDRH2 and a CDRH3 comprising the amino acid sequence of SEQ ID NOS: 110, 111 and 107, respectively and a VL region that contains a CDRL1, a CDRL2 and a CDRL3 comprising the amino acid sequence of SEQ ID NOS: 115, 116 and 117, respectively. In some embodiments, the antibody or antigen-binding fragment of the provided CAR contains a VH region that has a CDRH1, a CDRH2 and a CDRH3 comprising the amino acid sequence of SEQ ID NOS: 112, 113 and 114, respectively and a VL region that contains a CDRL1, a CDRL2 and a CDRL3 comprising the amino acid sequence of SEQ ID NOS: 118, 119 and 117, respectively. In some embodiments, the VH region comprises the sequence set forth in SEQ ID NO:25 and the VL region comprises the sequence set forth in SEQ ID NO:26. In some embodiments, the VH region comprises the sequence set forth in SEQ ID NO:25 and the VL region comprises the sequence set forth in SEQ ID NO:65. In some embodiments, the antibody or antigen-binding fragment is a single-chain antibody fragment, such as an scFv. In some embodiments, the scFv comprises the sequence of amino acids set forth in SEQ ID NO:5 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:5. In some embodiments, the scFv is encoded by a nucleotide sequence set forth in SEQ ID NO:261 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:261. In some embodiments, the scFv comprises the sequence of amino acids set forth in SEQ ID NO:6 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:6. In some embodiments, the scFv is encoded by a nucleotide sequence set forth in SEQ ID NO:262 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:262.

Among a provided anti-GPRC5D CAR is a CAR in which the antibody or antigen-binding fragment contains a VH region comprising the sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and contains a VL region comprising the sequence set forth in SEQ ID NO:28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:28. In some embodiments, the provided CAR is a CAR in which the antibody or antigen-binding fragment contains a VH region comprising the sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and contains a VL region comprising the sequence set forth in SEQ ID NO:66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:66. In some embodiments, the antibody or antigen-binding fragment of the provided CAR contains a VH region that has a CDRH1, a CDRH2 and a CDRH3 comprising the amino acid sequence of SEQ ID NOS: 120, 121 and 122, respectively and a VL region that has a CDRL1, a CDRL2 and a CDRL3 comprising the amino acid sequence of SEQ ID NOS: 130, 131 and 132, respectively. In some embodiments, the antibody or antigen-binding fragment of the provided CAR contains a VH region that has a CDRH1, a CDRH2 and a CDRH3 comprising the amino acid sequence of SEQ ID NOS: 123, 124 and 122, respectively and a VL region that contains a CDRL1, a CDRL2 and a CDRL3 comprising the amino acid sequence of SEQ ID NOS: 130, 131 and 132, respectively. In some embodiments, the antibody or antigen-binding fragment of the provided CAR contains a VH region that has a CDRH1, a CDRH2 and a CDRH3 comprising the amino acid sequence of SEQ ID NOS: 125, 126 and 122, respectively and a VL region that contains a CDRL1, a CDRL2 and a CDRL3 comprising the amino acid sequence of SEQ ID NOS: 130, 131 and 132, respectively. In some embodiments, the antibody or antigen-binding fragment of the provided CAR contains a VH region that has a CDRH1, a CDRH2 and a CDRH3 comprising the amino acid sequence of SEQ ID NOS: 127, 128 and 129, respectively and a VL region that contains a CDRL1, a CDRL2 and a CDRL3 comprising the amino acid sequence of SEQ ID NOS: 133, 134 and 132, respectively. In some embodiments, the VH region comprises the sequence set forth in SEQ ID NO:27 and the VL region comprises the sequence set forth in SEQ ID NO:28. In some embodiments, the VH region comprises the sequence set forth in SEQ ID NO:27 and the VL region comprises the sequence set forth in SEQ ID NO:66. In some embodiments, the antibody or antigen-binding fragment is a single-chain antibody fragment, such as an scFv. In some embodiments, the scFv comprises the sequence of amino acids set forth in SEQ ID NO:7 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:7. In some embodiments, the scFv is encoded by a nucleotide sequence set forth in SEQ ID NO:263 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:263. In some embodiments, the scFv comprises the sequence of amino acids set forth in SEQ ID NO:8 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:8. In some embodiments, the scFv is encoded by a nucleotide sequence set forth in SEQ ID NO:264 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:264.

Among a provided anti-GPRC5D CAR is a CAR in which the antibody or antigen-binding fragment contains a VH region comprising the sequence set forth in SEQ ID NO:29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:29; and contains a VL region comprising the sequence set forth in SEQ ID NO:30 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:30. In some embodiments, the provided CAR is a CAR in which the antibody or antigen-binding fragment contains a VH region comprising the sequence set forth in SEQ ID NO:29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:29; and contains a VL region comprising the sequence set forth in SEQ ID NO:67 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:67. In some embodiments, the antibody or antigen-binding fragment of the provided CAR contains a VH region that has a CDRH1, a CDRH2 and a CDRH3 comprising the amino acid sequence of SEQ ID NOS: 135, 136 and 137, respectively and a VL region that has a CDRL1, a CDRL2 and a CDRL3 comprising the amino acid sequence of SEQ ID NOS: 145, 146 and 147, respectively. In some embodiments, the antibody or antigen-binding fragment of the provided CAR contains a VH region that has a CDRH1, a CDRH2 and a CDRH3 comprising the amino acid sequence of SEQ ID NOS: 138, 139 and 137, respectively and a VL region that contains a CDRL1, a CDRL2 and a CDRL3 comprising the amino acid sequence of SEQ ID NOS: 145, 146 and 147, respectively. In some embodiments, the antibody or antigen-binding fragment of the provided CAR contains a VH region that has a CDRH1, a CDRH2 and a CDRH3 comprising the amino acid sequence of SEQ ID NOS: 140, 141 and 137, respectively and a VL region that contains a CDRL1, a CDRL2 and a CDRL3 comprising the amino acid sequence of SEQ ID NOS: 145, 146 and 147, respectively. In some embodiments, the antibody or antigen-binding fragment of the provided CAR contains a VH region that has a CDRH1, a CDRH2 and a CDRH3 comprising the amino acid sequence of SEQ ID NOS: 142, 143 and 144, respectively and a VL region that contains a CDRL1, a CDRL2 and a CDRL3 comprising the amino acid sequence of SEQ ID NOS: 148, 149 and 147, respectively. In some embodiments, the VH region comprises the sequence set forth in SEQ ID NO:29 and the VL region comprises the sequence set forth in SEQ ID NO:30. In some embodiments, the VH region comprises the sequence set forth in SEQ ID NO:29 and the VL region comprises the sequence set forth in SEQ ID NO:67. In some embodiments, the antibody or antigen-binding fragment is a single-chain antibody fragment, such as an scFv. In some embodiments, the scFv comprises the sequence of amino acids set forth in SEQ ID NO:9 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:9. In some embodiments, the scFv is encoded by a nucleotide sequence set forth in SEQ ID NO:265 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:265. In some embodiments, the scFv comprises the sequence of amino acids set forth in SEQ ID NO:10 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:10. In some embodiments, the scFv is encoded by a nucleotide sequence set forth in SEQ ID NO:266 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:266.

Among a provided anti-GPRC5D CAR is a CAR in which the antibody or antigen-binding fragment contains a VH region comprising the sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and contains a VL region comprising the sequence set forth in SEQ ID NO:32 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:32. In some embodiments, the provided CAR is a CAR in which the antibody or antigen-binding fragment contains a VH region comprising the sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and contains a VL region comprising the sequence set forth in SEQ ID NO:68 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:68. In some embodiments, the antibody or antigen-binding fragment of the provided CAR contains a VH region that has a CDRH1, a CDRH2 and a CDRH3 comprising the amino acid sequence of SEQ ID NOS: 135, 150 and 151, respectively and a VL region that has a CDRL1, a CDRL2 and a CDRL3 comprising the amino acid sequence of SEQ ID NOS: 157, 158 and 159, respectively. In some embodiments, the antibody or antigen-binding fragment of the provided CAR contains a VH region that has a CDRH1, a CDRH2 and a CDRH3 comprising the amino acid sequence of SEQ ID NOS: 152, 153 and 151, respectively and a VL region that contains a CDRL1, a CDRL2 and a CDRL3 comprising the amino acid sequence of SEQ ID NOS: 157, 158 and 159, respectively. In some embodiments, the antibody or antigen-binding fragment of the provided CAR contains a VH region that has a CDRH1, a CDRH2 and a CDRH3 comprising the amino acid sequence of SEQ ID NOS: 140, 154 and 151, respectively and a VL region that contains a CDRL1, a CDRL2 and a CDRL3 comprising the amino acid sequence of SEQ ID NOS: 157, 158 and 159, respectively. In some embodiments, the antibody or antigen-binding fragment of the provided CAR contains a VH region that has a CDRH1, a CDRH2 and a CDRH3 comprising the amino acid sequence of SEQ ID NOS: 142, 155 and 156, respectively and a VL region that contains a CDRL1, a CDRL2 and a CDRL3 comprising the amino acid sequence of SEQ ID NOS: 160, 161 and 159, respectively. In some embodiments, the VH region comprises the sequence set forth in SEQ ID NO:31 and the VL region comprises the sequence set forth in SEQ ID NO:32. In some embodiments, the VH region comprises the sequence set forth in SEQ ID NO:31 and the VL region comprises the sequence set forth in SEQ ID NO:68. In some embodiments, the antibody or antigen-binding fragment is a single-chain antibody fragment, such as an scFv. In some embodiments, the scFv comprises the sequence of amino acids set forth in SEQ ID NO:11 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:11. In some embodiments, the scFv is encoded by a nucleotide sequence set forth in SEQ ID NO:267 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:267. In some embodiments, the scFv comprises the sequence of amino acids set forth in SEQ ID NO:12 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:12. In some embodiments, the scFv is encoded by a nucleotide sequence set forth in SEQ ID NO:268 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:268.

Among a provided anti-GPRC5D CAR is a CAR in which the antibody or antigen-binding fragment contains a VH region comprising the sequence set forth in SEQ ID NO:33 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:33; and contains a VL region comprising the sequence set forth in SEQ ID NO:34 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:34. In some embodiments, the provided CAR is a CAR in which the antibody or antigen-binding fragment contains a VH region comprising the sequence set forth in SEQ ID NO:33 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:33; and contains a VL region comprising the sequence set forth in SEQ ID NO:69 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:69. In some embodiments, the antibody or antigen-binding fragment of the provided CAR contains a VH region that has a CDRH1, a CDRH2 and a CDRH3 comprising the amino acid sequence of SEQ ID NOS: 162, 163 and 164, respectively and a VL region that has a CDRL1, a CDRL2 and a CDRL3 comprising the amino acid sequence of SEQ ID NOS: 172, 86, 173, respectively. In some embodiments, the antibody or antigen-binding fragment of the provided CAR contains a VH region that has a CDRH1, a CDRH2 and a CDRH3 comprising the amino acid sequence of SEQ ID NOS: 165, 166 and 164, respectively and a VL region that contains a CDRL1, a CDRL2 and a CDRL3 comprising the amino acid sequence of SEQ ID NOS: 172, 86 and 173, respectively. In some embodiments, the antibody or antigen-binding fragment of the provided CAR contains a VH region that has a CDRH1, a CDRH2 and a CDRH3 comprising the amino acid sequence of SEQ ID NOS: 167, 168 and 164, respectively and a VL region that contains a CDRL1, a CDRL2 and a CDRL3 comprising the amino acid sequence of SEQ ID NOS: 172, 86 and 173, respectively. In some embodiments, the antibody or antigen-binding fragment of the provided CAR contains a VH region that has a CDRH1, a CDRH2 and a CDRH3 comprising the amino acid sequence of SEQ ID NOS: 169, 170, 171, respectively and a VL region that contains a CDRL1, a CDRL2 and a CDRL3 comprising the amino acid sequence of SEQ ID NOS: 174, 89 and 175, respectively. In some embodiments, the antibody or antigen-binding fragment of the provided CAR contains a VH region that has a CDRH1, a CDRH2 and a CDRH3 comprising the amino acid sequence of SEQ ID NOS: 169, 170, 171, respectively and a VL region that contains a CDRL1, a CDRL2 and a CDRL3 comprising the amino acid sequence of SEQ ID NOS: 174, 89 and 297, respectively. In some embodiments, the VH region comprises the sequence set forth in SEQ ID NO:33 and the VL region comprises the sequence set forth in SEQ ID NO:34. In some embodiments, the VH region comprises the sequence set forth in SEQ ID NO:33 and the VL region comprises the sequence set forth in SEQ ID NO:69. In some embodiments, the antibody or antigen-binding fragment is a single-chain antibody fragment, such as an scFv. In some embodiments, the scFv comprises the sequence of amino acids set forth in SEQ ID NO:13 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:13. In some embodiments, the scFv is encoded by a nucleotide sequence set forth in SEQ ID NO:269 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:269. In some embodiments, the scFv comprises the sequence of amino acids set forth in SEQ ID NO:14 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:14. In some embodiments, the scFv is encoded by a nucleotide sequence set forth in SEQ ID NO:270 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:270.

Among the antibodies, e.g., antigen-binding fragments, in the provided CARs, are human antibodies. In some embodiments of a provided human anti-GPRC5D antibody, e.g., antigen-binding fragments, the human antibody contains a VH region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a VL region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the VH region corresponds to the CDR-H1, CDR-H2 and/or CDR-H3. In some embodiments, the portion of the VH region corresponds to the framework region 1 (FR1), FR2, FR2 and/or FR4. In some embodiments, the portion of the VL region corresponds to the CDR-L1, CDR-L2 and/or CDR-L3. In some embodiments, the portion of the VL region corresponds to the FR1, FR2, FR2 and/or FR4.

In some embodiments, the human antibody, e.g., antigen-binding fragment, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody, e.g., antigen-binding fragment, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody, e.g., antigen-binding fragment, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody, e.g., antigen-binding fragment, contains a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody, e.g., antigen-binding fragment, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody, e.g., antigen-binding fragment, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody, e.g., antigen-binding fragment, contains a framework region that contains human germline gene segment sequences. For example, in some embodiments, the human antibody contains a VH region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a VL region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the VH region and/or VL region differs by no more than 10 amino acids, such as no more than 9, 8, 7, 6, 5, 4, 3, 2 or 1 amino acid, compared to the framework region sequence encoded by a human germline antibody segment.

In some embodiments the other recombinant receptor is an anti-BCMA receptor, such as an anti-BCMA CAR. Use or incorporation of any anti-BCMA CAR in the provided cells, methods and uses herein is contemplated. Polynucleotides encoding an anti-GPRC5D receptor provided herein and another receptor, for example in a multicistronic (e.g., bicistronic) expression vector, are likewise contemplated. Exemplary anti-BCMA CAR molecules are described in WO 2013/154760, WO 2015/052538, WO 2015/090229, WO 2015/092024, WO 2015/158671, WO 2016/014565, WO 2016/014789, WO 2016/094304, WO 2016/166630, WO 2017/021450, WO 2017/083511, WO 2017/130223, WO 2017/211900, WO 2018/085690, WO 2018/028647, WO 2019/090003.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human antibodies, and cells expressing such chimeric receptors have been previously described. See Carpenter et al., Clin Cancer Res., 2013, 19(8):2048-2060, WO 2016/090320, WO2016090327, WO2010104949A2 and WO2017173256. In some embodiments, the anti-BCMA CAR contains an antigen-binding domain, such as an scFv, containing a variable heavy (VH) and/or a variable light (VL) region derived from an antibody described in WO 2016/090320 or WO2016090327.

Among a provided anti-BCMA CAR is a CAR in which the antibody or antigen-binding fragment contains a VH region comprising the sequence set forth in SEQ ID NO: 189 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:189; and contains a VL region comprising the sequence set forth in SEQ ID NO:190 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:190. In some embodiments, the antibody or antigen-binding fragment of the provided CAR contains a VH region that has a CDRH1, a CDRH2 and a CDRH3 comprising the amino acid sequence of SEQ ID NOS: 199, 200, 201, respectively and a VL region that has a CDRL1, a CDRL2 and a CDRL3 comprising the amino acid sequence of SEQ ID NOS: 218, 219 and 220, respectively. In some embodiments, the VH region comprises the sequence set forth in SEQ ID NO:189 and the VL region comprises the sequence set forth in SEQ ID NO:190. In some embodiments, the antibody or antigen-binding fragment is a single-chain antibody fragment, such as an scFv. In some embodiments, the scFv comprises the sequence of amino acids set forth in SEQ ID NO:237 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:237. In some embodiments, the scFv is encoded by a nucleotide sequence set forth in SEQ ID NO:242 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:242. In some embodiments, the anti-BCMA CAR has the sequence of amino acids set forth in SEQ NO: 247 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:247.

Among a provided anti-BCMA CAR is a CAR in which the antibody or antigen-binding fragment contains a VH region comprising the sequence set forth in SEQ ID NO: 191 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:191; and contains a VL region comprising the sequence set forth in SEQ ID NO:192 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:192. In some embodiments, the antibody or antigen-binding fragment of the provided CAR contains a VH region that has a CDRH1, a CDRH2 and a CDRH3 comprising the amino acid sequence of SEQ ID NOS: 202, 203, 204, respectively and a VL region that has a CDRL1, a CDRL2 and a CDRL3 comprising the amino acid sequence of SEQ ID NOS: 221, 222, 223, respectively. In some embodiments, the VH region comprises the sequence set forth in SEQ ID NO:191 and the VL region comprises the sequence set forth in SEQ ID NO:192. In some embodiments, the antibody or antigen-binding fragment is a single-chain antibody fragment, such as an scFv. In some embodiments, the scFv comprises the sequence of amino acids set forth in SEQ ID NO:238 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:238. In some embodiments, the scFv is encoded by a nucleotide sequence set forth in SEQ ID NO:243 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:243. In some embodiments, the anti-BCMA CAR has the sequence of amino acids set forth in SEQ NO: 248 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:248.

Among a provided anti-BCMA CAR is a CAR in which the antibody or antigen-binding fragment contains a VH region comprising the sequence set forth in SEQ ID NO: 193 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:193; and contains a VL region comprising the sequence set forth in SEQ ID NO:194 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:194. In some embodiments, the antibody or antigen-binding fragment of the provided CAR contains a VH region that has a CDRH1, a CDRH2 and a CDRH3 comprising the amino acid sequence of SEQ ID NOS: 199, 200 and 205, respectively and a VL region that has a CDRL1, a CDRL2 and a CDRL3 comprising the amino acid sequence of SEQ ID NOS: 224, 225 and 226, respectively. In some embodiments, the VH region comprises the sequence set forth in SEQ ID NO:193 and the VL region comprises the sequence set forth in SEQ ID NO:194. In some embodiments, the antibody or antigen-binding fragment is a single-chain antibody fragment, such as an scFv. In some embodiments, the scFv comprises the sequence of amino acids set forth in SEQ ID NO:239 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:239. In some embodiments, the scFv is encoded by a nucleotide sequence set forth in SEQ ID NO:244 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:244. In some embodiments, the anti-BCMA CAR has the sequence of amino acids set forth in SEQ NO: 249 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:249.

Among a provided anti-BCMA CAR is a CAR in which the antibody or antigen-binding fragment contains a VH region comprising the sequence set forth in SEQ ID NO: 195 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:195; and contains a VL region comprising the sequence set forth in SEQ ID NO:196 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:196. In some embodiments, the antibody or antigen-binding fragment of the provided CAR contains a VH region that has a CDRH1, a CDRH2 and a CDRH3 comprising the amino acid sequence of SEQ ID NOS: 206, 207 and 208, respectively and a VL region that has a CDRL1, a CDRL2 and a CDRL3 comprising the amino acid sequence of SEQ ID NOS: 227, 228 and 229, respectively. In some embodiments, the antibody or antigen-binding fragment of the provided CAR contains a VH region that has a CDRH1, a CDRH2 and a CDRH3 comprising the amino acid sequence of SEQ ID NOS: 212, 213 and 214, respectively and a VL region that has a CDRL1, a CDRL2 and a CDRL3 comprising the amino acid sequence of SEQ ID NOS: 233, 234 and 229, respectively. In some embodiments, the VH region comprises the sequence set forth in SEQ ID NO:195 and the VL region comprises the sequence set forth in SEQ ID NO:196. In some embodiments, the antibody or antigen-binding fragment is a single-chain antibody fragment, such as an scFv. In some embodiments, the scFv comprises the sequence of amino acids set forth in SEQ ID NO:240 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:240. In some embodiments, the scFv is encoded by a nucleotide sequence set forth in SEQ ID NO:245 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:245. In some embodiments, the anti-BCMA CAR has the sequence of amino acids set forth in SEQ NO: 250 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:250.

Among a provided anti-BCMA CAR is a CAR in which the antibody or antigen-binding fragment contains a VH region comprising the sequence set forth in SEQ ID NO: 197 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:197; and contains a VL region comprising the sequence set forth in SEQ ID NO:198 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:198. In some embodiments, the antibody or antigen-binding fragment of the provided CAR contains a VH region that has a CDRH1, a CDRH2 and a CDRH3 comprising the amino acid sequence of SEQ ID NOS: 209, 210 and 211, respectively and a VL region that has a CDRL1, a CDRL2 and a CDRL3 comprising the amino acid sequence of SEQ ID NOS: 230, 231 and 232, respectively. In some embodiments, the antibody or antigen-binding fragment of the provided CAR contains a VH region that has a CDRH1, a CDRH2 and a CDRH3 comprising the amino acid sequence of SEQ ID NOS: 215, 216 and 217, respectively and a VL region that has a CDRL1, a CDRL2 and a CDRL3 comprising the amino acid sequence of SEQ ID NOS: 235, 236, 232, respectively. In some embodiments, the VH region comprises the sequence set forth in SEQ ID NO:197 and the VL region comprises the sequence set forth in SEQ ID NO:198. In some embodiments, the antibody or antigen-binding fragment is a single-chain antibody fragment, such as an scFv. In some embodiments, the scFv comprises the sequence of amino acids set forth in SEQ ID NO:241 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:241. In some embodiments, the scFv is encoded by a nucleotide sequence set forth in SEQ ID NO:246 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:246. In some embodiments, the anti-BCMA CAR has the sequence of amino acids set forth in SEQ NO: 251 or 252 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:251 or 252.

In some embodiments, the recombinant receptor such as a CAR comprising an anti-BCMA antibody (e.g., antigen-binding fragment) provided herein, further includes a spacer, such as any described in Section I.1.b. above.

In some embodiments, the recombinant receptor such as a CAR comprising an anti-BCMA antibody (e.g., antigen-binding fragment) provided herein, further includes a transmembrane domain, such as any described in Section I.1.c. above.

### III. Engineered Cells

Also provided are cells such as engineered cells that contain a recombinant receptor (e.g., a chimeric antigen receptor) such as one that contains an extracellular domain including an anti-GPRC5D receptor as provided herein. Also provided are populations of such cells, compositions containing such cells and/or enriched for such cells, such as in which cells expressing the GPRC5D-binding receptor make up at least 50, 60, 70, 80, 90, 91, 92, 93, 94, 95, 96, 97, 98, 99, or more percent of the total cells in the composition or cells of a certain type such as T cells, CD8+ cells or CD4+ cells.

Also provided are cells such as engineered cells that are engineered to contain a recombinant anti-GPRC5D receptor (e.g., an anti-GPRC5D CAR) and at least a second recombinant receptor. In some embodiments, the second receptor is an anti-BCMA receptor. In some embodiments, the second receptor is a CAR. In some embodiments, the anti-GPRC5D receptor is a CAR and the second receptor is a CAR. In some embodiments, the engineered cells contain a recombinant anti-GPRC5D receptor (e.g., an anti-GPRC5D CAR), as provided herein, and an anti-BCMA receptor (e.g., an anti-BCMA CAR). The anti-BCMA receptor can be any known anti-BCMA receptor, such as an anti-BCMA CAR described herein or elsewhere (see, e.g., WO 2013/154760, WO 2015/052538, WO 2015/090229, WO 2015/092024, WO 2015/158671, WO 2016/014565, WO 2016/014789, WO 2016/094304, WO 2016/166630, WO 2017/021450, WO 2017/083511, WO 2017/130223, WO 2017/211900, WO 2018/085690, WO 2018/028647). Exemplary anti-BCMA CARs are described in Section II. It is contemplated that any of the described anti-BCMA CARs can be used as a second CAR in any of the provided multi-targeting approaches with an anti-GPRC5D CAR to target both GPRC5D and BCMA.

In some embodiments, the engineered cells provided herein can be combined with one or more engineered cell population(s) expressing one or more other recombinant receptor(s). Such engineered cell populations can be formulated in the same or separate compositions. Among the compositions are pharmaceutical compositions and formulations for administration, such as for adoptive cell therapy. Also provided are therapeutic methods for administering any of the cells or compositions provided herein to subjects, e.g., patients.

Thus, also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a recombinant receptor described herein.

A. Vectors and Methods for Genetic Engineering

Also provided are methods, polynucleotides, compositions, and kits, for expressing the anti-GPRC5D recombinant receptors (e.g., CARs), and for producing the genetically engineered cells expressing such receptors. In some embodiments, one or more recombinant receptors (e.g., CARs) can be genetically engineered into cells or plurality of cells. The genetic engineering generally involves introduction of a nucleic acid encoding the recombinant or engineered component(s) into the cell, such as by lentiviral transduction, retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example, in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphoribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of polynucleotides encoding the receptors, including via viral, e.g., retroviral or lentiviral, transduction, transposons, and electroporation.

In some embodiments, recombinant polynucleotides are transferred into cells using recombinant infectious virus particles, such as, e.g., vectors derived from simian virus 40 (SV40), adenoviruses, adeno-associated virus (AAV). In some embodiments, recombinant polynucleotides are transferred into T cells using recombinant lentiviral vectors, such as HIV-1 lentivirus-based vectors (lentivectors; see, e.g., Amado et al., Science. 1999 Jul. 30; 285(5428):674-676), or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557).

In some embodiments, the retroviral vector or lentiviral vector has a long terminal repeat sequence (LTR). In some embodiments the vector is derived from the Moloney murine leukemia virus (MoMLV), myeloproliferative sarcoma virus (MPSV), murine embryonic stem cell virus (MESV), murine stem cell virus (MSCV), spleen focus forming virus (SFFV), human immunodeficiency virus type 1 (HIV-1), human immunodeficiency virus type 2 (HIV-2/SIV) or adeno-associated virus (AAV). In some embodiments, the vectors are self-inactivating (SIN). In some embodiments, the vectors are conditionally replicating (mobilizable) vectors. Most lentiviral vectors are derived from human, feline or simian lentiviruses. Most retroviral vectors are derived from murine retroviruses. In some embodiments, the lentiviruses or retroviruses include those derived from any avian or mammalian cell source. The lentiviruses or retroviruses typically are amphotropic, meaning that they are capable of infecting host cells of several species, including humans. In one embodiment, the gene to be expressed replaces the retroviral gag, pol and/or env sequences. Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. A number of illustrative retroviral systems have also been described (e.g., Amado et al., (1999) Science 285(5428):674-676, U.S. Pat. Nos. 5,219,740; 6,207,453; 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, recombinant polynucleotides are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant polynucleotides are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston (1990) Nature 346: 776-777); and strontium phosphate DNA co-precipitation (Brash et al., (1987) Mol. Cell Biol. 7: 2031-2034). Other approaches and vectors for transfer of the polynucleotides encoding the recombinant products are those described, e.g., in international patent application, Publication No.: WO2014055668, and U.S. Pat. No. 7,446,190.

Among additional polynucleotides, e.g., genes for introduction are those to improve the outcome of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments, one or more recombinant receptors (e.g., CARs) can be genetically engineered to be expressed in cells or plurality of cells. In some embodiments, a first recombinant receptor and a second binding molecule, e.g., recombinant receptor, are encoded by the same or separate nucleic acid molecules. In some embodiments, additional binding molecules are engineered to be expressed in cells or a plurality of cells. In some embodiments the second binding molecule is an anti-BCMA receptor, such as an anti-BCMA CAR described herein or in WO 2013/154760, WO 2015/052538, WO 2015/090229, WO 2015/092024, WO 2015/158671, WO 2016/014565, WO 2016/014789, WO 2016/094304, WO 2016/166630, WO 2017/021450, WO 2017/083511, WO 2017/130223, WO 2017/211900, WO 2018/085690, WO 2018/028647.

In some embodiments the vector or construct can contain a promoter and/or enhancer or regulatory elements to regulate expression of the encoded recombinant receptor. In some examples the promoter and/or enhancer or regulatory elements can be condition-dependent promoters, enhancers, and/or regulatory elements. In some examples these elements drive expression of the transgene. In some examples, the CAR transgene can be operatively linked to a promoter, such as an EF1alpha promoter with an HTLV1 enhancer (SEQ ID NO: 61). In some examples, the CAR transgene is operatively linked to a Woodchuck Hepatitis Virus (WHP) Posttranscriptional Regulatory Element (WPRE; SEQ ID NO: 62), located downstream of the transgene.

In some embodiments, the vector or construct can contain a single promoter that drives the expression of one or more nucleic acid molecules. In some embodiments, such nucleic acid molecules, e.g., transcripts, can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding a first and second chimeric receptor) by a message from a single promoter. For example, in some embodiments, the vector or construct can contain a nucleic acid encoding an anti-GPRC5D receptor (e.g., an anti-GPRC5D CAR) provided herein and a nucleic acid encoding an anti-BCMA receptor (e.g., an anti-BCMA CAR), separated by an IRES, under the regulation of a single promoter.

Alternatively, in some cases, a single promoter may direct expression of an RNA that contains, in a single open reading frame (ORF), two or three genes (e.g.encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A, e.g., SEQ ID NO: 42 or 43), equine rhinitis A virus (E2A, e.g., SEQ ID NO: 40 or 41), Thosea asigna virus (T2A, e.g., SEQ ID NO: 35, 36, or 37), and porcine teschovirus-1 (P2A, e.g., SEQ ID NO: 38 or 39) as described in U.S. Patent Publication No. 20070116690. In some embodiments, the one or more different or separate promoters drive the expression of one or more nucleic acid molecules encoding the one or more binding molecules, e.g., recombinant receptors.

Any of the recombinant receptors provided herein, e.g., anti-GPRC5D recombinant receptors and/or the additional recombinant receptors, can be encoded by polynucleotides containing one or more nucleic acid molecules encoding the receptors, in any combinations or arrangements. For example, one, two, three or more polynucleotides can encode one, two, three or more different receptors or domains. In some embodiments, one vector or construct contains nucleic acid molecules encoding one or more recombinant receptor(s), and a separate vector or construct contains nucleic acid molecules encoding an additional binding molecule, e.g., antibody and/or recombinant receptor, such as an anti-BCMA receptor (e.g., anti-BCMA CAR). Each of the nucleic acid molecules can also encode one or more surrogate marker(s), such as fluorescent protein (e.g., green fluorescent protein (GFP)) or a cell surface marker (e.g., a truncated surface marker such as truncated EGFR (tEGFR), which may be used to confirm transduction or engineering of the cell to express the receptor. For example, in some aspects, extrinsic marker genes are utilized in connection with engineered cell therapies to permit detection or selection of cells and, in some cases, also to promote cell suicide by ADCC. Exemplary marker genes include truncated epidermal growth factor receptor (EGFRt), which can be co-expressed with a transgene of interest (e.g., a CAR or TCR) in transduced cells (see, e.g., U.S. Pat. No. 8,802,374). EGFRt contains an epitope recognized by the antibody cetuximab (Erbitux®). For this reason, Erbitux® can be used to identify or select cells that have been engineered with the EGFRt construct, including in cells also co-engineered with another recombinant receptor, such as a chimeric antigen receptor (CAR).

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered.

In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Also provided are compositions containing one or more of the nucleic acid molecules, vectors or constructs, such as any described above. In some embodiments, the nucleic acid molecules, vectors, constructs or compositions can be used to engineer cells, such as T cells, to express any of the binding molecules, e.g., antibody or recombinant receptor, and/or the additional binding molecules.

B. Preparation of Cells for Engineering

In some embodiments, preparation of the engineered cells includes one or more culture and/or preparation steps. The cells for introduction of the recombinant receptor (e.g., CAR) may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using CD3/CD28 conjugated magnetic beads (e.g., DYNABEADS® M-450 CD3/CD28 T Cell Expander, MACSiBeads™, etc.).

In some embodiments, isolation is carried out by enrichment for a particular cell population by positive selection, and/or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase certain features, such as to improve long-term survival, expansion, and/or engraftment following administration, which in some aspects is particularly robust in such sub-populations (see Terakura et al. (2012) Blood. 1:72-82; Wang et al. (2012) J Immunother. 35(9):689-701). In some embodiments, combining TCM-enriched CD8+ T cells and CD4+ T cells further enhances response.

In embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA, and positive selection based on a marker characteristic of central memory T cells, such as CD62L or CCR7, where the positive and negative selections are carried out in either order.

CD4+T helper cells are sorted into naïve, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynabeads® or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084, are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS®) (Miltenyi Biotec, Auburn, Calif.). Magnetic Activated Cell Sorting (MACS®) systems are capable of high-purity selection of cells having magnetized particles attached thereto. In certain embodiments, MACS® operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS® system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS® system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy® system (Miltenyi Biotec). The CliniMACS Prodigy® system in some aspects is equipped with a cell processing unity that permits automated washing and fractionation of cells by centrifugation. The CliniMACS Prodigy® system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy® system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope (see, e.g., Klebanoff et al. (2012) J Immunother. 35(9): 651-660, Terakura et al. (2012) Blood. 1:72-82, and Wang et al. (2012) J Immunother. 35(9):689-701).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° C. per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

C. Engineered Cells, Vectors and Compositions for Multi-Targeting

Also provided are cells such as engineered cells that can bind to and/or target multiple antigens. In some embodiments, improved selectivity and specificity is achieved through strategies targeting multiple antigens. Such strategies generally involve multiple antigen-binding domains, which typically are present on distinct genetically engineered antigen receptors and specifically bind to distinct antigens. In some embodiments, the cells are engineered with the ability to bind more than one antigen. For example, in some embodiments, the cells are engineered to express multispecific binding molecules. In some embodiments, the cells express multiple binding molecules, e.g., recombinant receptors, each of which can target one antigen or multiple antigens, e.g., one receptor that targets GPRC5D, such as any described herein, and another receptor that targets another antigen, such as a tumor antigen, e.g., BCMA. Exemplary anti-BCMA receptors are described herein and in WO 2013/154760, WO 2015/052538, WO 2015/090229, WO 2015/092024, WO 2015/158671, WO 2016/014565, WO 2016/014789, WO 2016/094304, WO 2016/166630, WO 2017/021450, WO 2017/083511, WO 2017/130223, WO 2017/211900, WO 2018/085690, WO 2018/028647.

In some aspects, a plurality of genetically engineered antigen receptors are introduced into the cell, which specifically bind to different antigens, each expressed in or on the disease or condition to be targeted with the cells or tissues or cells thereof. Such features can in some aspects address or reduce the likelihood of off-target effects and/or increase response. For example, where a single antigen expressed in a disease or condition is also expressed on or in non-diseased or normal cells, such multi-targeting approaches can provide selectivity for desired cell types by requiring binding via multiple antigen receptors in order to activate the cell or induce a particular effector function. In some embodiments, a plurality of cells can be engineered to express one or more different binding molecules, e.g., recombinant receptors, each of which can target one antigen or multiple antigens.

Also provided are multispecific cells or compositions, such as those containing one or more of any of the binding molecules or cells provided herein. In some aspects, the multispecific cells such as cells containing a cell surface protein including the anti-GPRC5D receptor or domain thereof and an additional cell surface protein or domain thereof, such as an additional chimeric receptor or domain thereof, which binds to a different antigen or a different epitope on GPRC5D. In some embodiments, the additional chimeric receptor binds a BCMA antigen or an epitope of BCMA or the additional antigen is BCMA. In some embodiments, provided are compositions of cells that express recombinant receptors, wherein one or more of the binding molecules, multispecific binding molecules and/or recombinant receptors bind and/or target GPRC5D. In some embodiments, the multispecific binding molecules and/or recombinant receptors and/or cells or compositions target one or more different epitopes on GPRC5D. In some embodiments, the multispecific binding molecules and/or recombinant receptors or cells or compositions target one or more different epitopes on GPRC5D and one or more epitopes on BCMA.

In some embodiments, provided are composition of cells, wherein cells within the composition expresses one or more binding molecules, e.g., recombinant receptors. In some embodiments, the cell comprises (and in some cases has been transformed or transfected or transduced with) one or more vectors or constructs comprising one or more nucleic acid that encodes one or more an amino acid sequence comprising one or more antibodies and/or portions thereof, e.g., antigen-binding fragments thereof. In some embodiments, one or more such cells are provided. In some embodiments, a composition containing one or more such cells is provided. In some embodiments, the one or more cells can express different receptors or the same receptor. In some embodiments, cells within the composition express a multispecific binding molecule, e.g., a multispecific receptor, e.g., CAR.

In some aspects, the provided embodiments include multi-targeting strategies that target GPRC5D and a second or additional antigen associated with a particular disease or condition. In some embodiments, the second or additional antigen is targeted by a multispecific binding molecule and/or multiple binding molecules and/or a plurality of cells, e.g., one or more cells, each engineered to express one or more recombinant receptors. In some embodiments, a recombinant receptor targeting a second or additional antigen is expressed on the same cell as a GPRC5D binding molecule, e.g. an anti-GPRC5D CAR, or on a different cell.

In some embodiments, other recombinant receptors that specifically bind or target a second antigen are included in the cells, compositions, and methods provided herein. In some embodiments, the plurality of antigens, e.g., the first antigen, e.g., GPRC5D, and the second or additional antigens, e.g., BCMA, are expressed or suspected of being expressed on the cell, tissue, or disease or condition being targeted, such as on the cancer cell. In some aspects, the cell, tissue, disease or condition is multiple myeloma or a multiple myeloma cell.

In some aspects, the second antigen, e.g., or additional or further antigen, such as the disease-specific antigen and/or related antigen, is expressed on multiple myeloma, such as BCMA, CD38 (cyclic ADP ribose hydrolase), CD138 (syndecan-1, syndecan, SYN-1), CS-1 (CS1, CD2 subset 1, CRACC, SLAMF7, CD319, and 19A24), BAFF-R, TACI and/or FcRH5. Other exemplary multiple myeloma antigens include CD56, TIM-3, CD33, CD123, CD44, CD20, CD40, CD74, CD200, EGFR, 132-Microglobulin, HM1.24, IGF-1R, IL-6R, TRAIL-R1, and the activin receptor type IIA (ActRIIA) (see Benson and Byrd, J. Clin. Oncol. (2012) 30(16): 2013-15; Tao and Anderson, Bone Marrow Research (2011):924058; Chu et al., Leukemia (2013) 28(4):917-27; Garfall et al., Discov Med. (2014) 17(91):37-46). In some embodiments, the antigens include those present on lymphoid tumors, myeloma, AIDS-associated lymphoma, and/or post-transplant lymphoproliferations, such as CD38. Antibodies or antigen-binding fragments directed against such antigens are known and include, for example, those described in U.S. Pat. Nos. 8,153,765; 8,603,477, 8,008,450; U.S. Pub. No. US20120189622 or US20100260748; and/or International PCT Publication Nos. WO 2006/099875, WO 2009/080829, WO 2012/092612, WO2014210064, WO 2013/154760, WO 2015/052538, WO 2015/090229, WO 2015/092024, WO 2015/158671, WO 2016/014565, WO 2016/014789, WO 2016/094304, WO 2016/166630, WO 2017/021450, WO 2017/083511, WO 2017/130223, WO 2017/211900, WO 2018/085690, WO 2018/028647. In some embodiments, such antibodies or antigen-binding fragments thereof (e.g., scFv) are contained in multispecific antibodies, multispecific chimeric receptors, such as multispecific CARs, and/or multispecific cells.

In some embodiments, the provided cells express the provided CARs and one or more other recombinant receptors, such as an anti-BCMA recombinant receptor (e.g., an anti-BCMA CAR). In some embodiments, the provided cells contain polynucleotides that encode one or more other recombinant receptors (e.g., an anti-BCMA CAR) in addition to one or more polynucleotides encoding the anti-GPRC5D receptor provided herein. In some embodiments, the provided cells are combined with other engineered cells, expressing a recombinant receptor that is not an anti-GPRC5D receptor, such as engineered cells expressing an anti-BCMA receptor (e.g., an anti-BCMA CAR), in the same or separate compositions, and can administered together or separately in any of the provided methods or uses. Any recombinant receptor can be combined with the provided anti-GPRC5D receptor in any of the provided cells, such that the provided cells express both the anti-GPRC5D receptor provided herein and another receptor. Cells expressing an anti-GPRC5D receptor (e.g., anti-GPRC5D CAR) can be combined with cells expressing any recombinant receptor, in the same or separate composition, for use in the methods or uses provided herein. Such cells, compositions, and polynucleotides are described elsewhere herein.

In some embodiments the other recombinant receptor is an anti-BCMA receptor, such as an anti-BCMA CAR. Use or incorporation of any anti-BCMA CAR in the provided cells, methods and uses herein is contemplated. Polynucleotides encoding an anti-GPRC5D receptor provided herein and another receptor, for example in a multicistronic (e.g., bicistronic) expression vector, are likewise contemplated. Exemplary anti-BCMA CAR molecules are described in WO 2013/154760, WO 2015/052538, WO 2015/090229, WO 2015/092024, WO 2015/158671, WO 2016/014565, WO 2016/014789, WO 2016/094304, WO 2016/166630, WO 2017/021450, WO 2017/083511, WO 2017/130223, WO 2017/211900, WO 2018/085690, WO 2018/028647.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human antibodies, and cells expressing such chimeric receptors have been previously described. See Carpenter et al., Clin Cancer Res., 2013, 19(8):2048-2060, WO 2016/090320, WO2016090327, WO2010104949A2 and WO2017173256. In some embodiments, the anti-BCMA CAR contains an antigen-binding domain, such as an scFv, containing a variable heavy (VH) and/or a variable light (VL) region derived from an antibody described in WO 2016/090320 or WO2016090327.

Among a provided anti-BCMA CAR is a CAR in which the antibody or antigen-binding fragment contains a V_{H} region comprising the sequence set forth in SEQ ID NO: 189 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:189; and contains a V_{L} region comprising the sequence set forth in SEQ ID NO:190 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:190. In some embodiments, the antibody or antigen-binding fragment of the provided CAR contains a V_{H} region that has a CDRH1, a CDRH2 and a CDRH3 comprising the amino acid sequence of SEQ ID NOS: 199, 200, 201, respectively and a V_{L} region that has a CDRL1, a CDRL2 and a CDRL3 comprising the amino acid sequence of SEQ ID NOS: 218, 219 and 220, respectively. In some embodiments, the V_{H} region comprises the sequence set forth in SEQ ID NO:189 and the V_{L} region comprises the sequence set forth in SEQ ID NO:190. In some embodiments, the antibody or antigen-binding fragment is a single-chain antibody fragment, such as an scFv. In some embodiments, the scFv comprises the sequence of amino acids set forth in SEQ ID NO:237 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:237. In some embodiments, the scFv is encoded by a nucleotide sequence set forth in SEQ ID NO:242 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:242. In some embodiments, the anti-BCMA CAR has the sequence of amino acids set forth in SEQ NO: 247 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:247.

Among a provided anti-BCMA CAR is a CAR in which the antibody or antigen-binding fragment contains a V_{H} region comprising the sequence set forth in SEQ ID NO: 191 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:191; and contains a V_{L} region comprising the sequence set forth in SEQ ID NO:192 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:192. In some embodiments, the antibody or antigen-binding fragment of the provided CAR contains a V_{H} region that has a CDRH1, a CDRH2 and a CDRH3 comprising the amino acid sequence of SEQ ID NOS: 202, 203, 204, respectively and a V_{L} region that has a CDRL1, a CDRL2 and a CDRL3 comprising the amino acid sequence of SEQ ID NOS: 221, 222, 223, respectively. IIn some embodiments, the V_{H} region comprises the sequence set forth in SEQ ID NO:191 and the V_{L} region comprises the sequence set forth in SEQ ID NO:192. In some embodiments, the antibody or antigen-binding fragment is a single-chain antibody fragment, such as an scFv. In some embodiments, the scFv comprises the sequence of amino acids set forth in SEQ ID NO:238 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:238. In some embodiments, the scFv is encoded by a nucleotide sequence set forth in SEQ ID NO:243 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:243. In some embodiments, the anti-BCMA CAR has the sequence of amino acids set forth in SEQ NO: 248 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:248.

Among a provided anti-BCMA CAR is a CAR in which the antibody or antigen-binding fragment contains a V_{H} region comprising the sequence set forth in SEQ ID NO: 193 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:193; and contains a V_{L} region comprising the sequence set forth in SEQ ID NO:194 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:194. In some embodiments, the antibody or antigen-binding fragment of the provided CAR contains a V_{H} region that has a CDRH1, a CDRH2 and a CDRH3 comprising the amino acid sequence of SEQ ID NOS: 199, 200 and 205, respectively and a V_{L} region that has a CDRL1, a CDRL2 and a CDRL3 comprising the amino acid sequence of SEQ ID NOS: 224, 225 and 226, respectively. In some embodiments, the V_{H} region comprises the sequence set forth in SEQ ID NO:193 and the V_{L} region comprises the sequence set forth in SEQ ID NO:194. In some embodiments, the antibody or antigen-binding fragment is a single-chain antibody fragment, such as an scFv. In some embodiments, the scFv comprises the sequence of amino acids set forth in SEQ ID NO:239 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:239. In some embodiments, the scFv is encoded by a nucleotide sequence set forth in SEQ ID NO:244 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:244. In some embodiments, the anti-BCMA CAR has the sequence of amino acids set forth in SEQ NO: 249 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:249.

Among a provided anti-BCMA CAR is a CAR in which the antibody or antigen-binding fragment contains a V_{H} region comprising the sequence set forth in SEQ ID NO: 195 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:195; and contains a V_{L} region comprising the sequence set forth in SEQ ID NO:196 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:196. In some embodiments, the antibody or antigen-binding fragment of the provided CAR contains a V_{H} region that has a CDRH1, a CDRH2 and a CDRH3 comprising the amino acid sequence of SEQ ID NOS: 206, 207 and 208, respectively and a V_{L} region that has a CDRL1, a CDRL2 and a CDRL3 comprising the amino acid sequence of SEQ ID NOS: 227, 228 and 229, respectively. In some embodiments, the antibody or antigen-binding fragment of the provided CAR contains a V_{H} region that has a CDRH1, a CDRH2 and a CDRH3 comprising the amino acid sequence of SEQ ID NOS: 212, 213 and 214, respectively and a V_{L} region that has a CDRL1, a CDRL2 and a CDRL3 comprising the amino acid sequence of SEQ ID NOS: 233, 234 and 229, respectively. In some embodiments, the V_{H} region comprises the sequence set forth in SEQ ID NO:195 and the V_{L} region comprises the sequence set forth in SEQ ID NO:196. In some embodiments, the antibody or antigen-binding fragment is a single-chain antibody fragment, such as an scFv. In some embodiments, the scFv comprises the sequence of amino acids set forth in SEQ ID NO:240 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:240. In some embodiments, the scFv is encoded by a nucleotide sequence set forth in SEQ ID NO:245 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:245. In some embodiments, the anti-BCMA CAR has the sequence of amino acids set forth in SEQ NO: 250 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:250.

Among a provided anti-BCMA CAR is a CAR in which the antibody or antigen-binding fragment contains a V_{H} region comprising the sequence set forth in SEQ ID NO: 197 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:197; and contains a V_{L} region comprising the sequence set forth in SEQ ID NO:198 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:198. In some embodiments, the antibody or antigen-binding fragment of the provided CAR contains a V_{H} region that has a CDRH1, a CDRH2 and a CDRH3 comprising the amino acid sequence of SEQ ID NOS: 209, 210 and 211, respectively and a V_{L} region that has a CDRL1, a CDRL2 and a CDRL3 comprising the amino acid sequence of SEQ ID NOS: 230, 231 and 232, respectively. In some embodiments, the antibody or antigen-binding fragment of the provided CAR contains a V_{H} region that has a CDRH1, a CDRH2 and a CDRH3 comprising the amino acid sequence of SEQ ID NOS: 215, 216 and 217, respectively and a V_{L} region that has a CDRL1, a CDRL2 and a CDRL3 comprising the amino acid sequence of SEQ ID NOS: 235, 236, 232, respectively. In some embodiments, the V_{H} region comprises the sequence set forth in SEQ ID NO:197 and the V_{L} region comprises the sequence set forth in SEQ ID NO:198. In some embodiments, the antibody or antigen-binding fragment is a single-chain antibody fragment, such as an scFv. In some embodiments, the scFv comprises the sequence of amino acids set forth in SEQ ID NO:241 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:241. In some embodiments, the scFv is encoded by a nucleotide sequence set forth in SEQ ID NO:246 or a sequence of nucleotides at least at or about 90%, at or about 91%, at or about 92%, at or about 93%, at or about 94%, at or about 95%, at or about 96%, at or about 97%, at or about 98%, or at or about 99% identity to SEQ ID NO:246. In some embodiments, the anti-BCMA CAR has the sequence of amino acids set forth in SEQ NO: 251 or 252 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:251 or 252.

In some embodiments, the cells and methods include multi-targeting strategies, such as expression of two or more genetically engineered receptors on the cell, each recognizing a different antigen and in some aspects each including a different intracellular signaling component. Such multi-targeting strategies are described, for example, in International Patent Application, Publication No.: WO 2014/055668 (describing combinations of activating and costimulatory CARs, e.g., targeting two different antigens present individually on off-target, e.g., normal cells, but present together only on cells of the disease or condition to be treated) and Fedorov et al. (2013) Sci. Transl. Medicine, 5(215) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

In some embodiments, a plurality of cells, each engineered to express one or more recombinant receptors, is provided. For example, in some embodiments, one cell is engineered to express a binding molecule that binds and/or targets GPRC5D, and another cell is engineered to express a binding molecule that binds and/or targets an additional or second antigen. In some embodiments, the additional or second antigen is BCMA. In some embodiments, the cells can each express a multispecific binding molecule, e.g., a multispecific recombinant receptor, where one or more of the target antigen is GPRC5D. In some of such embodiments, the plurality of cells can be administered together or separately. In some embodiments, the plurality of cells is administered simultaneously, concurrently, or subsequently with the cells, e.g., administered on the same day, and/or sequentially with or intermittently with, in any order, another engineered cell in the plurality. For example, in some embodiments, an engineered cell expressing a GPRC5D-binding receptor, e.g., CAR, is administered simultaneously with or sequentially with, in any order, another engineered cell expressing a binding molecule that binds a different target antigen, e.g., BCMA, or a different epitope on GPRC5D. In some embodiments, the plurality of cells can be in the same composition.

### IV. Pharmaceutical Compositions

Also provided are compositions including the anti-GPRC5D recombinant receptors (e.g. anti-GPRC5D CARs), and engineered cells, including pharmaceutical compositions and formulations. Among such compositions are those that include engineered cells, such as a plurality of engineered cells, expressing the provided anti-GPRC5D recombinant receptors (e.g., CARs).

Also provided are compositions including the anti-GPRC5D recombinant receptors (e.g. anti-GPRC5D CARs) and a second recombinant receptor (e.g. CAR), such as an anti-BCMA recombinant receptor (e.g. anti-BCMA CAR), and engineered cells, including pharmaceutical compositions and formulations. Among such compositions are those that include engineered cells, such as a plurality of engineered cells, expressing the provided anti-GPRC5D recombinant receptors (e.g., CARs) and/or the second recombinant receptor (e.g. CAR), such as an anti-BCMA recombinant receptor (e.g. anti-BCMA CAR). In some embodiments, provided compositions include engineered cells, such as a plurality of engineered cells, expressing the provided anti-GPRC5D CARs and also expressing anti-BCMA CARs. In some embodiments, provided compositions include engineered cells, expressing the provided CARs that bind GPRC5D and BCMA, such as those comprising an anti-GPRC5D scFv and an anti-BCMA scFv.

Provided are pharmaceutical formulations comprising a GPRC5D-binding recombinant chimeric antigen receptor and engineered cells expressing said receptors, a plurality of engineered cells expressing said receptors and/or additional agents for combination treatment or therapy. The pharmaceutical compositions and formulations generally include one or more optional pharmaceutically acceptable carrier(s) or excipient(s). In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

Formulations of the antibodies described herein can include lyophilized formulations and aqueous solutions.

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the binding molecules and/or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells containing the binding molecules, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. In some aspects, in the context of genetically engineered cells expressing the binding molecules, e.g., CAR, a composition can contain at least the number of cells for administration for a dose of cell therapy, such as about or at least a number of cells described herein for administration.

The may be administered using standard administration techniques, formulations, and/or devices. Provided are formulations and devices, such as syringes and vials, for storage and administration of the compositions. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, intracranial, intrathoracic, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g., films, or microcapsules.

The formulations to be used for in vivo administration are generally sterile. Sterility may be readily accomplished, e.g., by filtration through sterile filtration membranes.

Also provided are pharmaceutical compositions for combination therapy. Any of the additional agents for combination therapy described herein, such as agents described in Section II.C, can be prepared and administered as one or more pharmaceutical compositions, with the anti-GPRC5D recombinant receptor (e.g., chimeric antigen receptor) and/or engineered cells expressing said molecules (e.g., recombinant receptor) described herein. The combination therapy can be administered in one or more pharmaceutical compositions, e.g., where the binding molecules, recombinant receptors and/or cells are in the same pharmaceutical composition as the additional agent, or in separate pharmaceutical compositions. For example, in some embodiments, the additional agent is an additional engineered cell, e.g., cell engineered to express a different recombinant receptor, such as BCMA-targeted recombinant receptor, and is administered in the same composition or in a separate composition. In some embodiments, each of the pharmaceutical composition is formulated in a suitable formulation according to the particular binding molecule, recombinant receptor, cell, e.g., engineered cell, and/or additional agent, and the particular dosage regimen and/or method of delivery.

### V. Methods and Uses

Also provided are methods of using and uses of the GPRC5D-targeted recombinant receptors, engineered cells, and pharmaceutical compositions and formulations thereof, such as in the treatment of diseases, conditions, and disorders in which GPRC5D is expressed, and/or detection, diagnostic, and prognostic methods. Among such methods and uses are those that involve administering to a subject engineered cells, such as a plurality of engineered cells, expressing the provided anti-GPRC5D recombinant receptors (e.g., CARs). Also provided are methods of combination therapy and/or treatment.

A. Therapeutic and Prophylactic Methods and Uses

Also provided are methods of administering and uses of, such as therapeutic and prophylactic uses of, the anti-GPRC5D recombinant receptors (e.g., CARs), engineered cells expressing the recombinant receptors (e.g., CARs), plurality of engineered cells expressing the receptors, and/or compositions comprising the same. Such methods and uses include therapeutic methods and uses, for example, involving administration of the molecules (e.g., recombinant receptors), cells (e.g., engineered cells), or compositions containing the same, to a subject having a disease, condition, or disorder associated with GPRC5D such as a disease, condition, or disorder associated with GPRC5D expression, and/or in which cells or tissues express, e.g., specifically express, GPRC5D. In some embodiments, the molecule, cell, and/or composition is/are administered in an effective amount to effect treatment of the disease or disorder. Provided herein are uses of the recombinant receptors (e.g., CARs), and cells (e.g., engineered cells) in such methods and treatments, and in the preparation of a medicament in order to carry out such therapeutic methods. In some embodiments, the methods are carried out by administering the binding molecules or cells, or compositions comprising the same, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with GPRC5D, such as use in a treatment regimen.

Also provided are methods of administering and uses of, such as therapeutic and prophylactic uses of, the anti-GPRC5D and anti-BCMA recombinant receptors (e.g., CARs), engineered cells expressing the recombinant receptors (e.g., CARs), plurality of engineered cells expressing the receptors, and/or compositions comprising the same. Such methods and uses include therapeutic methods and uses, for example, involving administration of the molecules (e.g., recombinant receptors), cells (e.g., engineered cells), or compositions containing the same, to a subject having a disease, condition, or disorder associated with GPRC5D and/or BCMA, such as a disease, condition, or disorder associated with GPRC5D and/or BCMA expression, and/or in which cells or tissues express, e.g., specifically express, GPRC5D and/or BCMA. In some embodiments, the molecule, cell, and/or composition is/are administered in an effective amount to effect treatment of the disease or disorder. Provided herein are uses of the recombinant receptors (e.g., CARs), and cells (e.g., engineered cells) in such methods and treatments, and in the preparation of a medicament in order to carry out such therapeutic methods. In some embodiments, the methods are carried out by administering the binding molecules or cells, or compositions comprising the same, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with GPRC5D and/or BCMA, such as use in a treatment regimen.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or subject being treated. As sufficient or significant delay can, in effect, encompass prevention, in that the subject does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, an antibody or composition or cell which suppresses tumor growth reduces the rate of growth of the tumor compared to the rate of growth of the tumor in the absence of the antibody or composition or cell.

An “effective amount” of an agent, e.g., a pharmaceutical formulation, binding molecule, antibody, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, binding molecule, antibody, cells, or composition refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the molecules, antibodies, cells, and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human.

Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

Among the diseases to be treated is any disease or disorder associated with GPRC5D or any disease or disorder in which GPRC5D is specifically expressed and/or in which GPRC5D has been targeted for treatment (also referred to herein interchangeably as a “GPRC5D-associated disease or disorder”). Cancers associated with GPRC5D expression include hematologic malignancies such as myeloma, e.g., multiple myeloma. In some embodiments, the disease or disorder associated with GPRC5D is a B cell-related disorder or malignancy. In some embodiments the disease or disorder associated with GPRC5D is multiple myeloma or Waldenstrom's Macroglobulinemia. In certain embodiments, the disease or disorder is multiple myeloma.

Among the diseases to be treated is any disease or disorder associated with GPRC5D and/or BCMA or any disease or disorder in which GPRC5D and/or BCMA is specifically expressed and/or in which GPRC5D and/or BCMA has been targeted for treatment (also referred to herein interchangeably as a “GPRC5D-associated disease or disorder” or a “BCMA-associated disease or disorder”). Cancers associated with GPRC5D and/or BCMA expression include hematologic malignancies such as myeloma, e.g., multiple myeloma. In some embodiments, the disease or disorder associated with GPRC5D and/or BCMA is a B cell-related disorder or malignancy. In some embodiments the disease or disorder associated with GPRC5D and/or BCMA is multiple myeloma or Waldenstrom's Macroglobulinemia. In certain embodiments, the disease or disorder is multiple myeloma.

In some embodiments, the disease or disorder is associated with expression of GPRC5D and BCMA. In some embodiments, cells of the disease are suspected of expressing both antigens. In some embodiments, one or both of the antigens is susceptible to antigen loss, in that some cells of the disease may no longer express both antigens. Thus, in some embodiments. a dual-targeting approach, targeting both GPRC5D and BCMA, may be advantageous.

In some embodiments, the methods may identify a subject who has, is suspected to have, or is at risk for developing a GPRC5D-associated disease or disorder. Hence, provided are methods for identifying subjects with diseases or disorders associated with GPRC5D expression and selecting them for treatment with a provided GPRC5D-binding recombinant receptors (e.g., CARs), and/or engineered cells expressing the recombinant receptors.

In some embodiments, the methods may identify a subject who has, is suspected to have, or is at risk for developing a GPRC5D- and/or BCMA-associated disease or disorder. Hence, provided are methods for identifying subjects with diseases or disorders associated with GPRC5D and/or BCMA expression and selecting them for treatment with provided GPRC5D-binding and BCMA-binding recombinant receptors (e.g., CARs), and/or engineered cells expressing the recombinant receptors.

For example, a subject may be screened for the presence of a disease or disorder associated with elevated GPRC5D expression, such as a GPRC5D-expressing cancer. In some embodiments, the methods include screening for or detecting the presence of a GPRC5D-associated disease, e.g., a tumor. Thus, in some aspects, a sample may be obtained from a patient suspected of having a disease or disorder associated with elevated GPRC5D expression and assayed for the expression level of GPRC5D. In some aspects, a subject who tests positive for a GPRC5D-associated disease or disorder may be selected for treatment by the present methods, and may be administered a therapeutically effective amount of a recombinant receptor (e.g., CAR) comprising a GPRC5D-binding molecule, cells containing a recombinant receptor, or a pharmaceutical composition thereof as described herein.

For example, a subject may be screened for the presence of a disease or disorder associated with elevated GPRC5D and/or BCMA expression, such as a GPRC5D- and/or BCMA-expressing cancer. In some embodiments, the methods include screening for or detecting the presence of a GPRC5D- and/or BCMA associated disease, e.g., a tumor. Thus, in some aspects, a sample may be obtained from a patient suspected of having a disease or disorder associated with elevated GPRC5D and/or BCMA expression and assayed for the expression level of GPRC5D and/or BCMA. In some aspects, a subject who tests positive for a GPRC5D- and/or BCMA-associated disease or disorder may be selected for treatment by the present methods, and may be administered a therapeutically effective amount of a composition comprising cells expressing a recombinant receptor (e.g., CAR) comprising a GPRC5D-binding molecule and expressing a recombinant receptor comprising a BCMA-binding molecule, or a pharmaceutical composition thereof as described herein. In some aspects, a subject who tests positive for a GPRC5D- and/or BCMA-associated disease or disorder may be selected for treatment by the present methods, and may be administered a therapeutically effective amount of a composition comprising cells expressing a recombinant receptor (e.g., CAR) comprising a GPRC5D-binding molecule and a BCMA-binding molecule, cells expressing a recombinant receptor comprising a GPRC5D-binding molecule and a recombinant receptor comprising a BCMA-binding molecule, or a pharmaceutical composition thereof as described herein.

In some embodiments, the subject has persistent or relapsed disease, e.g., following treatment with a GPRC5D-specific antibody and/or cells expressing a GPRC5D-targeting chimeric receptor and/or other therapy, including chemotherapy, radiation, and/or hematopoietic stem cell transplantation (HSCT), e.g., allogenic HSCT or or autologous HSCT. In some embodiments, the administration effectively treats the subject despite the subject having become resistant to another GPRC5D-targeted therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the subject has received a prior therapy that is a BCMA CAR therapy or other BCMA-targeted therapy. In some embodiments, the subject is refractory to or has relapsed following such BCMA CAR therapy or other BCMA-targeted therapy. In some cases, the subject is refractory to or has relapsed due to BCMA antigen-negative tumor cells and/or BCMA antigen/epitope loss following therapy.

In some embodiments, the subject has persistent or relapsed disease following treatment with another therapy, such as treatment with a BCMA-specific antibody, BCMA-targeting receptor, and/or cells expressing a BCMA-targeting chimeric receptor. In some embodiments, the administration effectively treats the subject despite the subject having become resistant to another therapy, such as a BCMA-targeted therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the subject is one that is eligible for a transplant, such as is eligible for a hematopoietic stem cell transplantation (HSCT), e.g., allogenic HSCT or autologous HSCT. In some of such embodiments, the subject has not previously received a transplant, despite being eligible, prior to administration of the anti-GPRC5D recombinant receptors (e.g., CARs), engineered cells expressing the recombinant receptors (e.g., CARs), plurality of engineered cells expressing the receptors, and/or compositions comprising the same, as provided herein.

In some embodiments, the subject is one that is not eligible for a transplant, such as is not eligible for a hematopoietic stem cell transplantation (HSCT), e.g., allogenic HSCT or or autologous HSCT. In some of such embodiments, such a subject is administered the anti-GPRC5D recombinant receptors (e.g., CARs), engineered cells expressing the recombinant receptors (e.g., CARs), plurality of engineered cells expressing the receptors, and/or compositions comprising the same, according to the provided embodiments herein.

In some embodiments, prior to the initiation of administration of the engineered cells, the subject has received one or more prior therapies. In some embodiments, the subject has received at least 1, 2, 3, 4, 5, 6, 7, 8, 9, 10, 11, 12, 13, 14, 15, 16, 17, 18, 19 or 20 or more prior therapies. In some embodiments, the subject has received at least 3, 4, 5, 6, 7, 8, 9, 10 or more prior therapies.

In some aspects, the subject has relapsed following, or has been refractory to, one or more of, for example, each, individually, of the one or more prior therapies. In some aspects, the prior therapies include treatment with autologous stem cell transplant (ASCT); an immunomodulatory agent; a proteasome inhibitor; and an anti-CD38 antibody; unless the subject was not a candidate for or was contraindicated for one or more of the therapies. In some embodiments, the immunomodulatory agent is selected from among thalidomide, lenalidomide or pomalidomide. In some embodiments, the proteasome inhibitor is selected from among bortezomib, carfilzomib or ixazomib. In some embodiments, the anti-CD38 antibody is or comprises daratumumab. In some embodiments, the subject must have undergone at least 2 consecutive cycles of treatment for each regimen unless progressive disease was the best response to the regimen.

In some embodiments, the method can involve including or excluding particular subjects for therapy with the provided anti-GPRC5D antibodies, recombinant receptors and/or cells comprising such receptors, based on particular criteria, diagnosis or indication. In some embodiments, at the time of administration of the dose of cells, the subject has not had active or history of plasma cell leukemia (PCL). In some embodiments, if the subject had active or a history of PCL at the time of administration, the subject can be excluded from being treated according to the provided methods. In some embodiments, if the subject develops a PCL, such as secondary PCL, at the time of administration, the subject can be excluded from being treated according to the provided methods. In some embodiments, the assessment for the criteria, diagnosis or indication can be performed at the time of screening the subjects for eligibility or suitability of treatment according to the provided methods, at various steps of the treatment regimen, at the time of receiving lymphodepleting therapy, and/or at or immediately prior to the initiation of administration of the engineered cells or composition thereof.

In some embodiments, the method can involve including or excluding particular subjects for therapy with the provided anti-GPRC5D and anti-BCMA antibodies, recombinant receptors and/or cells comprising such receptors, based on particular criteria, diagnosis or indication. In some embodiments, at the time of administration of the dose of cells, the subject has not had active or history of plasma cell leukemia (PCL). In some embodiments, if the subject had active or a history of PCL at the time of administration, the subject can be excluded from being treated according to the provided methods. In some embodiments, if the subject develops a PCL, such as secondary PCL, at the time of administration, the subject can be excluded from being treated according to the provided methods. In some embodiments, the assessment for the criteria, diagnosis or indication can be performed at the time of screening the subjects for eligibility or suitability of treatment according to the provided methods, at various steps of the treatment regimen, at the time of receiving lymphodepleting therapy, and/or at or immediately prior to the initiation of administration of the engineered cells or composition thereof.

In some embodiments, the treatment does not induce an immune response by the subject to the therapy, and/or does not induce such a response to a degree that prevents effective treatment of the disease or condition. In some aspects, the degree of immunogenicity and/or graft versus host response is less than that observed with a different but comparable treatment. For example, in the case of adoptive cell therapy using cells expressing CARs including the provided anti-GPRC5D antibodies, the degree of immunogenicity in some embodiments is reduced compared to CARs including a different antibody that binds to a similar, e.g., overlapping epitope and/or that competes for binding to GPRC5D with the antibody, such as a mouse or monkey or rabbit or humanized antibody.

In some embodiments, the methods include adoptive cell therapy, whereby genetically engineered cells expressing the provided recombinant receptors comprising a GPRC5D-binding molecule (e.g., CARs comprising anti-GPRC5D antibody or antigen-binding fragment thereof) are administered to subjects. Such administration can promote activation of the cells (e.g., T cell activation) in a GPRC5D-targeted manner, such that the cells of the disease or disorder are targeted for destruction.

In some embodiments, the methods include adoptive cell therapy, whereby genetically engineered cells expressing the provided recombinant receptors comprising a GPRC5D-binding molecule (e.g., CARs comprising anti-GPRC5D antibody or antigen-binding fragment thereof) and genetically engineered cells expressing the provided recombinant receptors comprising a BCMA-binding molecule (e.g., CARs comprising anti-BCMA antibody or antigen-binding fragment thereof) are administered to subjects. In some embodiments, the methods include adoptive cell therapy, whereby genetically engineered cells expressing the provided recombinant receptors comprising a GPRC5D-binding molecule and a BCMA-binding molecule (e.g., CARs comprising anti-GPRC5D and anti-BCMA antibodies or antigen-binding fragments thereof).

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition, such as by lessening tumor burden in a GPRC5D-expressing cancer.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered, is a primate, such as a human. In some embodiments, the subject, to whom the cells, cell populations, or compositions are administered, is a non-human primate. In some embodiments, the non-human primate is a monkey (e.g., cynomolgus monkey) or an ape. The subject can be male or female and can be any suitable age, including infant, juvenile, adolescent, adult, and geriatric subjects. In some embodiments, the subject is a non-primate mammal, such as a rodent (e.g., mouse, rat, etc.). In some examples, the patient or subject is a validated animal model for disease, adoptive cell therapy, and/or for assessing toxic outcomes such as cytokine release syndrome (CRS).

The GPRC5D-binding recombinant receptors (e.g., CARs) and cells expressing the same, can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. The GPRC5D-binding recombinant receptors, BCMA-binding recombinant receptors, and GPRC5D- and BCMA-binding recombinant receptors (e.g., CARs) and cells expressing the same, can be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intracranial, intrathoracic, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule, recombinant receptor or cell may depend on the type of disease to be treated, the type of binding molecule or recombinant receptor, the severity and course of the disease, whether the binding molecule or recombinant receptor is administered for preventive or therapeutic purposes, previous therapy, the patient's clinical history and response to the recombinant receptor or cell, and the discretion of the attending physician. The compositions and molecules and cells are in some embodiments suitably administered to the patient at one time or over a series of treatments.

In some embodiments, the dose and/or frequency of administration is/are determined based on efficacy and/or response. In some embodiments, efficacy is determined by evaluating disease status. Exemplary methods for assessing disease status include: measurement of M protein in biological fluids, such as blood and/or urine, by electrophoresis and immunofixation; quantification of sFLC (κ and λ) in blood; skeletal survey; and imaging by positron emission tomography (PET)/computed tomography (CT) in subjects with extramedullary disease. In some embodiments, disease status can be evaluated by bone marrow examination.

In some examples, dose and/or frequency of administration is determined by the expansion and persistence of the recombinant receptor or cell in the blood and/or bone marrow. In some embodiments, dose and/or frequency of administration is determined based on the antitumor activity of the recombinant receptor or engineered cell. In some embodiments antitumor activity is determined by the overall response rate (ORR) and/or International Myeloma Working Group (IMWG) Uniform Response Criteria (see Kumar et al. (2016) Lancet Oncol 17(8):e328-346). In some embodiments, response is evaluated using minimal residual disease (MRD) assessment. In some embodiments, MRD can be assessed by methods such as flow cytometry and high-throughput sequencing, e.g., deep sequencing. In some embodiments, response is evaluated based on the duration of response following administration of the recombinant receptor or cells. In some examples, dose and/or frequency of administration can be based on toxicity. In some embodiments, dose and/or frequency can be determined based on health-related quality of life (HRQoL) of the subject to which the recombinant receptor and/or cells is/are administered. In some embodiments, dose and/or frequency of administration can be changed, i.e., increased or decreased, based on any of the above criteria.

In some embodiments, the disease or disorder to be treated is multiple myeloma. In some embodiments, measurable disease criteria for multiple myeloma can include (1) serum M-protein 1 g/dL or greater; (2) Urine M-protein 200 mg or greater/24 hour; (3) involved serum free light chain (sFLC) level 10 mg/dL or greater, with abnormal κ to λ ratio. In some cases, light chain disease is acceptable only for subjects without measurable disease in the serum or urine.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sorensen et al., (1993) Br J Cancer 67(4) 773-775. In some embodiments, the subject that are to be administered according to the methods or treatment regimen provided herein include those with an ECOG performance status of 0 or 1.

In some embodiments, the administration can treat the subject despite the subject having become resistant to another therapy. In some embodiments, when administered to subjects according to the embodiments described herein, the dose or the composition is capable of achieving objective response (OR), in at least 50%, 60%, 70%, 80%, 90%, or 95% of subjects that were administered. In some embodiments, OR includes subjects who achieve stringent complete response (sCR), complete response (CR), very good partial response (VGPR), partial response (PR) and minimal response (MR). In some embodiments, when administered to subjects according to the embodiments described herein, the dose or the composition is capable of achieving stringent complete response (sCR), complete response (CR), very good partial response (VGPR) or partial response (PR), in at least 50%, 60%, 70%, 80%, or 85% of subjects that were administered. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered. In some embodiments, exemplary doses include about 5.0×10⁷, 1.5×10⁸, 3.0×10⁸ or 4.5×10⁸ CAR-expressing T cells. In some embodiments, exemplary doses include about 1.0×10⁷, 1.25×10⁷, 1.5×10⁷, 2.0×10⁷, 2.0×10⁷, 2.5×10⁷, 3.0×10⁷, 3.5×10⁷, 4.0×10⁷, 4.5×10⁷, 5.0×10⁷, 7.5×10⁷, 1.5×10⁸, 2.25×10⁸, 3.0×10⁸, 4.5×10⁸, or 6.0×10⁸ CAR-expressing T cells. In some aspects, particular response to the treatment, e.g., according to the methods provided herein, can be assessed based on the International Myeloma Working Group (IMWG) Uniform Response Criteria (see Kumar et al. (2016) Lancet Oncol 17(8):e328-346). In some embodiments, exemplary doses to achieve particular outcomes, such as OR, includes about 5.0×10⁷ CAR-expressing T cells.

In some embodiments, toxicity and/or side-effects of treatment can be monitored and used to adjust dose and/or frequency of administration of the recombinant receptor, e.g., CAR, cells, and or compositions. For example, adverse events and laboratory abnormalities can be monitored and used to adjust dose and/or frequency of administration. Adverse events include infusion reactions, cytokine release syndrome (CRS), neurotoxicity, macrophage activation syndrome, and tumor lysis syndrome (TLS). Any of such events can establish dose-limiting toxicities and warrant decrease in dose and/or a termination of treatment. Other side effects or adverse events which can be used as a guideline for establishing dose and/or frequency of administration include non-hematologic adverse events, which include but are not limited to fatigue, fever or febrile neutropenia, increase in transaminases for a set duration (e.g., less than or equal to 2 weeks or less than or equal to 7 days), headache, bone pain, hypotension, hypoxia, chills, diarrhea, nausea/vomiting, neurotoxicity (e.g., confusion, aphasia, seizures, convulsions, lethargy, and/or altered mental status), disseminated intravascular coagulation, other asymptomatic non-hematological clinical laboratory abnormalities, such as electrolyte abnormalities. Other side effects or adverse events which can be used as a guideline for establishing dose and/or frequency of administration include hematologic adverse events, which include but are not limited to neutropenia, leukopenia, thrombocytopenia, animal, and/or B-cell aplasia and hypogammaglobinemia.

In some embodiments, treatment according to the provided methods can result in a lower rate and/or lower degree of toxicity, toxic outcome or symptom, toxicity-promoting profile, factor, or property, such as a symptom or outcome associated with or indicative of cytokine release syndrome (CRS) or neurotoxicity, such as severe CRS or severe neurotoxicity, for example, compared to administration of other therapies.

In certain embodiments, in the context of genetically engineered cells containing the binding molecules or recombinant receptors, a subject is administered the range of about one million to about 100 billion cells and/or that amount of cells per kilogram of body weight, such as, e.g., about 1 million to about 50 billion cells (e.g., about 5 million cells, about 10 million, about 12.5 million, about 15 million, about 20 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 10 million cells, about 12.5 million cells, about 15 million cells, 20 million cells, about 25 million cells, about 30 million cells, about 40 million cells, about 50 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 150 million cells, about 250 million cells, about 300 million cells, about 350 million cells, about 450 million cells, about 500 million cells, about 600 million cells, about 650 million cells, about 800 million cells, about 900 million cells, about 1 billion cells, about 1.2 billion cells, about 3 billion cells, about 30 billion cells, about 45 billion cells, or about 50 billion cells) or any value in between these ranges and/or per kilogram of body weight. Again, dosages may vary depending on attributes particular to the disease or disorder and/or patient and/or other treatments.

In some embodiments, the methods comprise administering a dose of the engineered cells or a composition comprising a dose of the engineered cells. In some embodiments, the engineered cells or compositions containing engineered cells can be used in a treatment regimen, wherein the treatment regimen comprises administering a dose of the engineered cells or a composition comprising a dose of the engineered cells. In some embodiments, the dose can contain, for example, a particular number or range of recombinant receptor-expressing T cells, total T cells, or total peripheral blood mononuclear cells (PBMCs), such as any number of such cells described herein. In some embodiments, a composition containing a dose of the cells can be administered. In some aspects, the number, amount or proportion of CAR-expressing cells in a cell population or a cell composition can be assessed by detection of a surrogate marker, e.g., by flow cytometry or other means, or by detecting binding of a labelled molecule, such as a labelled antigen, that can specifically bind to the binding molecules or receptors provided herein.

In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 2.5×10⁷ to about 1.2×10⁹ such cells, such as 2.5×10⁷, 5×10⁷, 1.5×10⁸, 3×10⁸, 4.5×10⁸, 8×10⁸, or 1.2×10⁹ total such cells, or the range between any two of the foregoing values. In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 1.0×10⁷ to about 1.2×10⁹ such cells, such as 1.0×10⁷, 1.25×10⁷, 1.5×10⁷, 2.0×10⁷, 2.5×10⁷, 5×10⁷, 7.5×10⁷, 1.5×10⁸, 2.25×10⁸, 3×10⁸, 4.5×10⁸, 6.0×10⁸, 8×10⁸, or 1.2×10⁹ total such cells, or the range between any two of the foregoing values. In some embodiments, for example, where the subject is a human, the dose includes more than about 1×10⁶ total recombinant receptor (e.g., CAR)-expressing cells, T cells, or peripheral blood mononuclear cells (PBMCs) and fewer than about 2×10⁹ total recombinant receptor (e.g., CAR)-expressing cells, T cells, or peripheral blood mononuclear cells (PBMCs), e.g., in the range of about 1.0×10⁷ to about 6.5×10⁸ such cells, about 1.5×10⁷ to about 6.0×10⁸ such cells, about 1.5×10⁷ to about 6.5×10⁸ such cells, about 2.5×10⁷ to about 6.0×10⁸ such cells, or about about 5.0×10⁷ to about 6.0×10⁸ such cells.

In some embodiments, the dose of genetically engineered cells comprises between at or about 2.5×10⁷ CAR-expressing T cells, total T cells, or total peripheral blood mononuclear cells (PBMCs), and at or about 1.2×10⁹ CAR-expressing T cells, total T cells, or total PBMCs, between at or about 5.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 2.5×10⁷ to or to about 1.2×10⁹ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive.

In some embodiments, the dose of genetically engineered cells comprises between at or about 1.0×10⁷ CAR-expressing T cells, total T cells, or total peripheral blood mononuclear cells (PBMCs), and at or about 1.2×10⁹ CAR-expressing T cells, total T cells, or total PBMCs, between at or about 2.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 1.0×10⁷ to or to about 1.2×10⁹ CD3+ or CD8+ total T cells or CD3+ or CD8+ CAR-expressing cells, from or from about 1.5×10⁷ to or to about 1.2×10⁹ CD3+ or CD8+ total T cells or CD3+ or CD8+ CAR-expressing cells, from or from about 2.5×10⁷ to or to about 1.2×10⁹ CD3+ or CD8+ total T cells or CD3+ or CD8+ CAR-expressing cells, from or from about 1.5×10⁷ to or to about 8.0×10⁸ CD3+ or CD8+ total T cells or CD3+ or CD8+ CAR-expressing cells, from or from about 2.5×10⁷ to or to about 8.0×10⁸ CD3+ or CD8+ total T cells or CD3+ or CD8+ CAR-expressing cells, from or from about 1.5×10⁷ to or to about 6.0×10⁸ CD3+ or CD8+ total T cells or CD3+ or CD8+ CAR-expressing cells, from or from about 2.5×10⁷ to or to about 6.0×10⁸ CD3+ or CD8+ total T cells or CD3+ or CD8+ CAR-expressing cells, from or from about 5.0×10⁷ to or to about 6.0×10⁸ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between at or about 1×10⁶ and at or about 2×10⁹ total recombinant receptor (e.g., CAR)-expressing CD8+ cells, e.g., in the range of at or about 5×10⁷ to at or about 4.5×10⁸ such cells, such as at or about 2.5×10⁷, at or about 5×10⁷, at or about 1.5×10⁸, at or about 3×10⁸, at or about 4.5×10⁸, at or about 8×10⁸, or at or about 1.2×10⁹ total such cells, or the range between any two of the foregoing values.

In some embodiments, for example, where the subject is human, the CD8+ T cells of the dose, including in a dose including CD4+ and CD8+ T cells, includes between at or about 1×10⁶ and at or about 2×10⁹ total recombinant receptor (e.g., CAR)-expressing CD8+ cells, e.g., in the range of at or about 1×10⁷ to at or about 4.5×10⁸ such cells, such as at or about 1.0×10⁷, at or about 1.25×10⁷, at or about 1.5×10⁷, at or about 2.0×10⁷, at or about 2.5×10⁷, at or about 5×10⁷, at or about 7.5×10⁷, at or about 1.5×10⁸, at or about 3×10⁸, at or about 4.5×10⁸, at or about 6.0×10⁸, at or about 8×10⁸, or at or about 1.2×10⁹ total such cells, or the range between any two of the foregoing values.

In some embodiments, the dose of cells, e.g., recombinant receptor-expressing T cells, is administered to the subject as a single dose or is administered only one time within a period of two weeks, one month, three months, six months, 1 year or more. In some embodiments, the patient is administered multiple doses, and each of the doses or the total dose can be within any of the foregoing values.

In some embodiments, the engineered cells for administration or composition of engineered cells for administration, exhibits properties indicative of or consistent with cell health. In some embodiments, at or about or at least at or about 70, 75, 80, 85, or 90% CAR+ cells of such dose exhibit one or more properties or phenotypes indicative of cell health or biologically active CAR cell, such as absence expression of an apoptotic marker.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death, including blebbing, cell shrinkage, nuclear fragmentation, chromatin condensation, chromosomal DNA fragmentation, and global mRNA decay. In some aspects, early stages of apoptosis can be indicated by activation of certain caspases, e.g., 2, 8, 9, and 10. In some aspects, middle to late stages of apoptosis are characterized by further loss of membrane integrity, chromatin condensation and DNA fragmentation, include biochemical events such as activation of caspases 3, 6, and 7.

In particular embodiments, the phenotype is negative expression of one or more factors associated with programmed cell death, for example pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In certain embodiments, the phenotype is the absence of an indicator, e.g., an Annexin V molecule or by TUNEL staining, that will preferentially bind to cells undergoing apoptosis when incubated with or contacted to a cell composition. In some embodiments, the phenotype is or includes the expression of one or more markers that are indicative of an apoptotic state in the cell. In some embodiments, the phenotype is lack of expression and/or activation of a caspase, such as caspase 3. In some aspects, activation of caspase-3 is indicative of an increase or revival of apoptosis. In certain embodiments, caspase activation can be detected by known methods. In some embodiments, an antibody that binds specifically to an activated caspase (i.e., binds specifically to the cleaved polypeptide) can be used to detect caspase activation. In particular embodiments, the phenotype is or includes active caspase 3−. In some embodiments, the marker of apoptosis is a reagent that detects a feature in a cell that is associated with apoptosis. In certain embodiments, the reagent is an annexin V molecule.

In some embodiments, the compositions containing the engineered cells for administration contain a certain number or amount of cells that exhibit phenotypes indicative of or consistent with cell health. In some of any embodiments, less than about 25%, 20%, 15%, 10%, 9%, 8%, 7%, 6%, 5%, 4%, 3%, 2% or 1% of the CAR-expressing T cells in the dose of engineered T cells express a marker of apoptosis, optionally Annexin V or active Caspase 3. In some of any embodiments, less than 5%, 4%, 3%, 2% or 1% of the CAR-expressing T cells in the dose of engineered T cells express Annexin V or active Caspase 3.

In some embodiments the cells administered are immune cells engineered to express the GPRC5D-binding recombinant receptor, e.g., CAR. In some embodiments the immune cells are T cells. In some embodiments, the administered cells are CD4+ T cells. In some embodiments the administered cells are CD8+ T cells. In some embodiments, the administered cells are a combination of CD4+ and CD8+ T cells, such as CAR T cells. In some examples the ratio of CD4+ cells to CD8+ cells (CD4:CD8) is 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1.

In some embodiments, the cells, binding molecules, or recombinant receptors are administered as part of a combination treatment, such as simultaneously with or sequentially with, in any order, another therapeutic intervention, such as another antibody or engineered cell or receptor or agent, such as a cytotoxic or therapeutic agent.

The cells, binding molecules and/or recombinant receptors in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells, binding molecules and/or recombinant receptors are administered prior to the one or more additional therapeutic agents. In some embodiments, the cells, binding molecules and/or recombinant receptors are administered after to the one or more additional therapeutic agents.

In some embodiments, the subject may receive a bridging therapy after leukapheresis and before lymphodepleting chemotherapy. A treating physician can determine if bridging therapy is necessary, for example for disease control, during manufacturing of the provided composition or cells. In some embodiments, bridging therapies do not include biological agents, such as antibodies (e.g., Daratumumab). In some embodiments, bridging therapies are discontinued prior to initiation of lymphodepletion. In some embodiments, bridging therapies are discontinued 1 day, 2 days 3 days, 4 days, 5 days, 7 days, 10 days, 14 days, 21 days, 28 days, 45 days, or 60 days before lymphodepletion.

Once the cells are administered to a mammal (e.g., a human), the biological activity of the engineered cell populations and/or antibodies in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load.

In certain embodiments, engineered cells are modified in any number of ways, such that their therapeutic or prophylactic efficacy is increased. For example, the engineered CAR or TCR expressed by the population in some embodiments are conjugated either directly or indirectly through a linker to a targeting moiety. The practice of conjugating compounds, e.g., the CAR or TCR, to targeting moieties is known in the art. See, for instance, Wadwa et al., J. Drug Targeting, 3(2):111 (1995), and U.S. Pat. No. 5,087,616.

B. Combination Therapy

Also provided are methods of combination therapy that include administration and uses, such as therapeutic and prophylactic uses, of the GPRC5D-binding recombinant receptors (e.g., CARs), engineered cells expressing the recombinant receptors (e.g., CARs), plurality of engineered cells expressing the receptors, and/or compositions comprising the same. Also provided are methods of combination therapy that include administration and uses, such as therapeutic and prophylactic uses, of the GPRC5D-binding and BCMA-binding recombinant receptors (e.g., CARs), engineered cells expressing the recombinant receptors (e.g., CARs), plurality of engineered cells expressing the receptors, and/or compositions comprising the same.

In some embodiments, the GPRC5D-binding recombinant receptor (e.g., CAR) and/or engineered cells expressing said molecules (e.g., recombinant receptor) described herein are administered as part of a combination treatment or combination therapy, such as simultaneously with, sequentially with, or intermittently with, in any order, one or more additional therapeutic intervention. In some embodiments, the one or more additional therapeutic intervention includes, for example, an antibody, an engineered cell, a receptor and/or an agent, such as a cell expressing a recombinant receptor, and/or cytotoxic or therapeutic agent, e.g., a chemotherapeutic agent. In some embodiments, the combination therapy includes administration of one or more additional agents, therapies and/or treatments, e.g., any of the additional agents, therapy and/or treatments described herein. In some embodiments, the combination therapy includes administration of one or more additional agents for treatment or therapy, such as an immunomodulatory agent, immune checkpoint inhibitor, adenosine pathway or adenosine receptor antagonist or agonist and kinase inhibitors. In some embodiments, the combination treatment or combination therapy includes an additional treatment, such as a surgical treatment, transplant, and/or radiation therapy. Also provided are methods of combination treatment or combination therapy that includes GPRC5D-binding recombinant receptors (e.g., CARs), cells and/or compositions described herein and one or more additional therapeutic interventions.

In some embodiments, the GPRC5D-binding recombinant receptor, the BCMA-binding recombinant receptor, GPRC5D- and BCMA-binding recombinant receptor (e.g., CAR), and/or engineered cells expressing said molecules (e.g., recombinant receptor) described herein are administered as part of a combination treatment or combination therapy, such as simultaneously with, sequentially with, or intermittently with, in any order, one or more additional therapeutic intervention. In some embodiments, the one or more additional therapeutic intervention includes, for example, an antibody, an engineered cell, a receptor and/or an agent, such as a cell expressing a recombinant receptor, and/or cytotoxic or therapeutic agent, e.g., a chemotherapeutic agent. In some embodiments, the combination therapy includes administration of one or more additional agents, therapies and/or treatments, e.g., any of the additional agents, therapy and/or treatments described herein. In some embodiments, the combination therapy includes administration of one or more additional agents for treatment or therapy, such as an immunomodulatory agent, immune checkpoint inhibitor, adenosine pathway or adenosine receptor antagonist or agonist and kinase inhibitors. In some embodiments, the combination treatment or combination therapy includes an additional treatment, such as a surgical treatment, transplant, and/or radiation therapy. Also provided are methods of combination treatment or combination therapy that includes GPRC5D-binding recombinant receptors, BCMA-binding recombinant receptors, GPRC5D- and BCMA-binding recombinant receptors (e.g., CARs), cells and/or compositions described herein and one or more additional therapeutic interventions.

In some embodiments, the additional agent for combination treatment or combination therapy enhances, boosts and/or promotes the efficacy and/or safety of the therapeutic effect of binding molecules, recombinant receptors, cells and/or compositions. In some embodiments, the additional agent enhances or improves the efficacy, survival or persistence of the administered cells, e.g., cells expressing the binding molecule or a recombinant receptor. In some embodiments, the additional agent is selected from among a protein phosphatase inhibitor, a kinase inhibitor, a cytokine, an immunomodulator, or an agent that decreases the level or activity of a regulatory T (Treg) cell. In some embodiments, the additional agent enhances safety, by virtue of reducing or ameliorating adverse effects of the administered binding molecules, recombinant receptors, cells and/or compositions. In some embodiments, the additional agent can treat the same disease, condition or a comorbidity. In some embodiments, the additional agent can ameliorate, reduce or eliminate one or more toxicities, adverse effects or side effects that are associated with administration of the recombinant receptors, cells and/or compositions, e.g., CAR-expressing cells.

In some embodiments, pain management medication such as acetaminophen, or antihistamine, such as diphenhydramine can be administered prior to, during or after administration of the recombinant receptor, cell or composition provided herein, to ameliorate or reduce or eliminate minor side effects associated with treatment. In some examples, red blood cell and platelet transfusions, and/or colony-stimulating factors can be administered reduce or eliminate one or more toxicities, adverse effects or side effects that are associated with administration of the recombinant receptors, cells and/or compositions, e.g., CAR-expressing cells. In some embodiments, prophylactic or empiric anti-infective agents (e.g., trimethoprim/sulfamethoxazole for pneumocystis pneumonia [PCP] prophylaxis, broad spectrum antibiotics, antifungals, or antiviral agents for febrile neutropenia) can be administered to treat side-effects resulting from treatment. In some examples, when necessary, prophylaxis may be provided to treat lymphopenia and/or neutropenia occurring as a result of treatment.

In some embodiments, the additional therapy, treatment or agent includes chemotherapy, radiation therapy, surgery, transplantation, adoptive cell therapy, antibodies, cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, immune checkpoint inhibitors, antibiotics, angiogenesis inhibitors, metabolic modulators or other therapeutic agents or any combination thereof. In some embodiments, the additional agent is a protein, a peptide, a nucleic acid, a small molecule agent, a cell, a toxin, a lipid, a carbohydrate or combinations thereof, or any other type of therapeutic agent, e.g., radiation. In some embodiments, the additional therapy, agent or treatment includes surgery, chemotherapy, radiation therapy, transplantation, administration of cells expressing a recombinant receptor, e.g., CAR, kinase inhibitor, immune checkpoint inhibitor, mTOR pathway inhibitor, immunosuppressive agents, immunomodulators, antibodies, immunoablative agents, antibodies and/or antigen binding fragments thereof, antibody conjugates, other antibody therapies, cytotoxins, steroids, cytokines, peptide vaccines, hormone therapy, antimetabolites, metabolic modulators, drugs that inhibit either the calcium dependent phosphatase calcineurin or the p70S6 kinase FK506) or inhibit the p70S6 kinase, alkylating agents, anthracyclines, vinca alkaloids, proteasome inhibitors, GITR agonists, protein tyrosine phosphatase inhibitors, protein kinase inhibitors, an oncolytic virus, and/or other types of immunotherapy. In some embodiments, the additional agent or treatment is bone marrow transplantation, T cell ablative therapy using chemotherapy agents such as, fludarabine, external-beam radiation therapy (XRT), cyclophosphamide, and/or antibody therapy.

In some embodiments, the cells, GPRC5D-binding recombinant receptors and/or compositions, e.g., CAR-expressing cells, are administered in combination with other engineered cells, e.g., other CAR-expressing cells. In some embodiments, the cells, GPRC5D-binding recombinant receptors and/or compositions, e.g., CAR-expressing cells, are administered in combination with an additional agent. In some embodiments, the cells, GPRC5D-binding recombinant receptors and/or compositions, e.g., CAR-expressing cells, are administered in combination with other engineered cells, e.g., other CAR-expressing cells, as well as in combination with an additional agent. In some embodiments, the additional agent is a kinase inhibitor, e.g., an inhibitor of Bruton's tyrosine kinase (Btk), e.g., ibrutinib. In some embodiments, the additional agent is an adenosine pathway or adenosine receptor antagonist or agonist. In some embodiments, the additional agent is an immunomodulator such as thalidomide or a thalidomide derivative (e.g., lenalidomide). In some embodiments, the additional agent is a gamma secretase inhibitor, such as a gamma secretase inhibitor that inhibits or reduces intramembrane cleavage of a target of a gamma secretase, e.g., GPRC5D, on a cell (such as a tumor/cancer cell). In some embodiments, the additional therapy, agent or treatment is a cytotoxic or chemotherapy agent, a biologic therapy (e.g., antibody, e.g., monoclonal antibody, or cellular therapy), or an inhibitor (e.g., kinase inhibitor).

In some embodiments, the cells, GPRC5D-binding recombinant receptors, BCMA-binding recombinant receptors, GPRC5D- and BCMA-binding recombinant receptors, and/or compositions, e.g., CAR-expressing cells, are administered in combination with other engineered cells, e.g., other CAR-expressing cells. In some embodiments, the cells, GPRC5D-binding recombinant receptors, BCMA-binding recombinant receptors, GPRC5D- and BCMA-binding recombinant receptors, and/or compositions, e.g., CAR-expressing cells, are administered in combination with an additional agent. In some embodiments, the cells, GPRC5D-binding recombinant receptors, BCMA-binding recombinant receptors, GPRC5D- and BCMA-binding recombinant receptors, and/or compositions, e.g., CAR-expressing cells, are administered in combination with other engineered cells, e.g., other CAR-expressing cells, as well as in combination with an additional agent. In some embodiments, the additional agent is a kinase inhibitor, e.g., an inhibitor of Bruton's tyrosine kinase (Btk), e.g., ibrutinib. In some embodiments, the additional agent is an adenosine pathway or adenosine receptor antagonist or agonist. In some embodiments, the additional agent is an immunomodulator such as thalidomide or a thalidomide derivative (e.g., lenalidomide). In some embodiments, the additional agent is a gamma secretase inhibitor, such as a gamma secretase inhibitor that inhibits or reduces intramembrane cleavage of a target of a gamma secretase, e.g., GPRC5D and/or BCMA, on a cell (such as a tumor/cancer cell). In some embodiments, the additional therapy, agent or treatment is a cytotoxic or chemotherapy agent, a biologic therapy (e.g., antibody, e.g., monoclonal antibody, or cellular therapy), or an inhibitor (e.g., kinase inhibitor).

In some embodiments, the additional agent is a chemotherapeutic agent. Exemplary chemotherapeutic agents include an anthracycline (e.g., doxorubicin, such as liposomal doxorubicin); a vinca alkaloid (e.g., vinblastine, vincristine, vindesine, vinorelbine); an alkylating agent (e.g., cyclophosphamide, decarbazine, melphalan, ifosfamide, temozolomide); an immune cell antibody (e.g., alemtuzumab, gemtuzumab, rituximab, tositumomab); an antimetabolite (including, e.g., folic acid antagonists, pyrimidine analogs, purine analogs and adenosine deaminase inhibitors such as fludarabine); a TNFR glucocorticoid induced TNFR related protein (GITR) agonist; a proteasome inhibitor (e.g., aclacinomycin A, gliotoxin or bortezomib); an immunomodulatory such as thalidomide or a thalidomide derivative (e.g., lenalidomide).

In some embodiments, the additional therapy or treatment is cell therapy, e.g., adoptive cell therapy. In some embodiments, the additional therapy includes administration of engineered cells, e.g., additional CAR-expressing cell. In some embodiments, the additional engineered cell is a CAR-expressing cell that expresses the same or different recombinant receptor as the engineered cells provided herein, e.g., anti-GPRC5D CAR-expressing cells. In some embodiments, the recombinant receptor, e.g., CAR, expressed on the additional engineered cell, recognizes a different antigen and/or epitope. In some embodiments, the recombinant receptor, e.g., CAR, expressed on the additional engineered cell, recognizes a different epitope of the same antigen as the recombinant receptors described herein, e.g., GPRC5D. In some embodiments, the recombinant receptor, e.g., CAR, expressed on the additional engineered cell, recognizes a different antigen, e.g., a different tumor antigen or combination of antigens. For example, in some embodiments, the recombinant receptor, e.g., CAR, expressed on the additional engineered cell, targets cancer cells that express early lineage markers, e.g., cancer stem cells, while other CAR-expressing cells target cancer cells that express later lineage markers. In such embodiments, the additional engineered cell is administered prior to, concurrently with, or after administration (e.g., infusion) of the CAR-expressing cells described herein. In some embodiments, the additional engineered cell expresses an allogeneic CAR.

In some embodiments, the configurations of one or more of the CAR molecules comprise a primary intracellular signaling domain and two or more, e.g., 2, 3, 4, or 5 or more, costimulatory signaling domains. In some embodiments, the one or more of the CAR molecules may have the same or a different primary intracellular signaling domain, the same or different costimulatory signaling domains, or the same number or a different number of costimulatory signaling domains. In some embodiments, the one or more of the CAR molecules can be configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

In some embodiments, the additional agent is any of the cells engineered to express one or more of the anti-GPRC5D binding molecules and/or cells engineered to express additional binding molecules, e.g., recombinant receptors, e.g., CAR, that target a different antigen. In some embodiments, the additional agent includes any of the cells or plurality of cells described herein, e.g., in Section I.C. In some embodiments, the additional agent is a cell engineered to express a recombinant receptor, e.g., CAR, targeting a different epitope and/or antigen, e.g., a different antigen associated with a disease or condition. In some embodiments, the additional agent is a cell engineered to express a recombinant receptor, e.g., CAR, targeting a second or additional antigen expressed in multiple myeloma, e.g., CD38, CD138, CS-1, BAFF-R, TACI and/or FcRH5.

In some embodiments, the additional agent is an immunomodulatory agent. In some embodiments, the combination therapy includes an immunomodulatory agent that can stimulate, amplify and/or otherwise enhance an anti-tumor immune response, e.g., anti-tumor immune response from the administered engineered cells, such as by inhibiting immunosuppressive signaling or enhancing immunostimulant signaling. In some embodiments, the immunomodulatory agent is a peptide, protein or is a small molecule. In some embodiments, the protein can be a fusion protein or a recombinant protein. In some embodiments, the immunomodulatory agent binds to an immunologic target, such as a cell surface receptor expressed on immune cells, such a T cells, B cells or antigen-presenting cells. For example, in some embodiments, the immunomodulatory agent is an antibody or antigen-binding antibody fragment, a fusion protein, a small molecule or a polypeptide. In some embodiments, the recombinant receptors, cells and/or compositions are administered in combination with an additional agent that is an antibody or an antigen-binding fragment thereof, such as a monoclonal antibody.

In some embodiments, the immunomodulatory agent blocks, inhibits or counteracts a component of the immune checkpoint pathway. The immune system has multiple inhibitory pathways that are involved in maintaining self-tolerance and for modulating immune responses. Tumors can use certain immune-checkpoint pathways as a major mechanism of immune resistance, particularly against T cells that are specific for tumor antigens (Pardoll (2012) Nature Reviews Cancer 12:252-264), e.g., engineered cells such as CAR-expressing cells. Because many such immune checkpoints are initiated by ligand-receptor interactions, they can be readily blocked by antibodies against the ligands and/or their receptors.

Therefore, therapy with antagonistic molecules blocking an immune checkpoint pathway, such as small molecules, nucleic acid inhibitors (e.g., RNAi) or antibody molecules, are becoming promising avenues of immunotherapy for cancer and other diseases. In contrast to the majority of anti-cancer agents, checkpoint inhibitors do not necessarily target tumor cells directly, but rather target lymphocyte receptors or their ligands in order to enhance the endogenous antitumor activity of the immune system.

As used herein, the term “immune checkpoint inhibitor” refers to molecules that totally or partially reduce, inhibit, interfere with or modulate one or more checkpoint proteins. Checkpoint proteins regulate T-cell activation or function. These proteins are responsible for co-stimulatory or inhibitory interactions of T-cell responses Immune checkpoint proteins regulate and maintain self-tolerance and the duration and amplitude of physiological immune responses. In some embodiments, the subject can be administered an additional agent that can enhance or boost the immune response, e.g., immune response effected by the GPRC5D-binding recombinant receptors, cells and/or compositions provided herein, against a disease or condition, e.g., a cancer, such as any described herein.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors, ligands and/or receptor-ligand interaction. In some embodiments, modulation, enhancement and/or stimulation of particular receptors can overcome immune checkpoint pathway components. Illustrative immune checkpoint molecules that may be targeted for blocking, inhibition, modulation, enhancement and/or stimulation include, but are not limited to, PD-1 (CD279), PD-L1 (CD274, B7-H1), PDL2 (CD273, B7-DC), CTLA-4, LAG-3 (CD223), TIM-3, 4-1BB (CD137), 4-1BBL (CD137L), GITR (TNFRSF18, AITR), CD40, OX40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+(αβ) T cells), CD160 (also referred to as BY55), CGEN-15049, CEACAM (e.g., CEACAM-1, CEACAM-3 and/or CEACAM-5), TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and a transforming growth factor receptor (TGFR; e.g., TGFR beta) Immune checkpoint inhibitors include antibodies, or antigen binding fragments thereof, or other binding proteins, that bind to and block or inhibit and/or enhance or stimulate the activity of one or more of any of the said molecules.

Exemplary immune checkpoint inhibitors include Tremelimumab (CTLA-4 blocking antibody, also known as ticilimumab, CP-675,206), anti-OX40, PD-L1 monoclonal antibody (Anti-B7-H1; MEDI4736), MK-3475 (PD-1 blocker), nivolumab (anti-PD-1 antibody), CT-011 (anti-PD-1 antibody), BY55 monoclonal antibody, AMP224 (anti-PD-L1 antibody), BMS-936559 (anti-PD-L1 antibody), MPLDL3280A (anti-PD-L1 antibody), MSB0010718C (anti-PD-L1 antibody) and ipilimumab (anti-CTLA-4 antibody, also known as Yervoy®, MDX-010 and MDX-101). Exemplary immunomodulatory antibodies include, but are not limited to, Daclizumab (Zenapax), Bevacizumab (Avastin Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), IPH2201, ARGX-115, Emactuzumab, CC-90002 and MNRP1685A or an antibody-binding fragment thereof. Other exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon gamma, CAS 951209-71-5, available from IRX Therapeutics).

Programmed cell death 1 (PD-1) is an immune checkpoint protein that is expressed in B cells, NK cells, and T cells (Shinohara et al., 1995, Genomics 23:704-6; Blank et al., 2007, Cancer Immunol Immunother 56:739-45; Finger et al., 1997, Gene 197:177-87; Pardoll (2012) Nature Reviews Cancer 12:252-264). The major role of PD-1 is to limit the activity of T cells in peripheral tissues during inflammation in response to infection, as well as to limit autoimmunity. PD-1 expression is induced in activated T cells and binding of PD-1 to one of its endogenous ligands acts to inhibit T-cell activation by inhibiting stimulatory kinases. PD-1 also acts to inhibit the TCR “stop signal”. PD-1 is highly expressed on Treg cells and may increase their proliferation in the presence of ligand (Pardoll (2012) Nature Reviews Cancer 12:252-264). Anti-PD 1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Exemplary anti-PD-1 antibodies include nivolumab (Opdivo by BMS), pembrolizumab (Keytruda by Merck), pidilizumab (CT-011 by Cure Tech), lambrolizumab (MK-3475 by Merck), and AMP-224 (Merck), nivolumab (also referred to as Opdivo, BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are described in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are described in WO2009/101611. Pembrolizumab (formerly known as lambrolizumab, and also referred to as Keytruda, MK03475; Merck) is a humanized IgG4 monoclonal antibody that binds to PD-1. Pembrolizumab and other humanized anti-PD-1 antibodies are described in U.S. Pat. No. 8,354,509 and WO2009/114335. Other anti-PD-1 antibodies include AMP 514 (Amplimmune), among others, e.g., anti-PD-1 antibodies described in U.S. Pat. No. 8,609,089, US 2010028330, US 20120114649 and/or US 20150210769. AMP-224 (B7-DCIg; Amplimmune; e.g., described in WO2010/027827 and WO2011/066342), is a PD-L2 Fc fusion soluble receptor that blocks the interaction between PD-1 and B7-H1. PD-L1 (also known as CD274 and B7-H1) and PD-L2 (also known as CD273 and B7-DC) are ligands for PD-1, found on activated T cells, B cells, myeloid cells, macrophages, and some types of tumor cells. Anti-tumor therapies have focused on anti-PD-L1 antibodies. The complex of PD-1 and PD-L1 inhibits proliferation of CD8+ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., 2012, N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). Exemplary anti-PD-L1 antibodies include MDX-1105 (Medarex), MEDI4736 (Medimmune) MPDL3280A (Genentech), BMS-935559 (Bristol-Myers Squibb) and MSB0010718C. MEDI4736 (Medimmune) is a human monoclonal antibody that binds to PD-L1, and inhibits interaction of the ligand with PD-1. MDPL3280A (Genentech/Roche) is a human Fc optimized IgG1 monoclonal antibody that binds to PD-L1. MDPL3280A and other human monoclonal antibodies to PD-L1 are described in U.S. Pat. No. 7,943,743 and U.S. Publication No. 20120039906. Other anti-PD-L1 binding agents include YW243.55.570 (see WO2010/077634) and MDX-1105 (also referred to as BMS-936559, and, e.g., anti-PD-L1 binding agents described in WO2007/005874).

Cytotoxic T-lymphocyte-associated antigen (CTLA-4), also known as CD152, is a co-inhibitory molecule that functions to regulate T-cell activation. CTLA-4 is a member of the immunoglobulin superfamily that is expressed exclusively on T-cells. CTLA-4 acts to inhibit T-cell activation and is reported to inhibit helper T-cell activity and enhance regulatory T-cell immunosuppressive activity. Although the precise mechanism of action of CTLA-4 remains under investigation, it has been suggested that it inhibits T cell activation by outcompeting CD28 in binding to CD80 and CD86, as well as actively delivering inhibitor signals to the T cell (Pardoll (2012) Nature Reviews Cancer 12:252-264). Anti-CTLA-4 antibodies have been used in clinical trials for the treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). A significant feature of anti-CTLA-4 is the kinetics of anti-tumor effect, with a lag period of up to 6 months after initial treatment required for physiologic response. In some cases, tumors may actually increase in size after treatment initiation, before a reduction is seen (Pardoll (2012) Nature Reviews Cancer 12:252-264). Exemplary anti-CTLA-4 antibodies include ipilimumab (Bristol-Myers Squibb) and tremelimumab (Pfizer). Ipilimumab has recently received FDA approval for treatment of metastatic melanoma (Wada et al., 2013, J Transl Med 11:89).

Lymphocyte activation gene-3 (LAG-3), also known as CD223, is another immune checkpoint protein. LAG-3 has been associated with the inhibition of lymphocyte activity and in some cases the induction of lymphocyte anergy. LAG-3 is expressed on various cells in the immune system including B cells, NK cells, and dendritic cells. LAG-3 is a natural ligand for the MHC class II receptor, which is substantially expressed on melanoma-infiltrating T cells including those endowed with potent immune-suppressive activity. Exemplary anti-LAG-3 antibodies include BMS-986016 (Bristol-Myers Squib), which is a monoclonal antibody that targets LAG-3. IMP701 (Immutep) is an antagonist LAG-3 antibody and IMP731 (Immutep and GlaxoSmithKline) is a depleting LAG-3 antibody. Other LAG-3 inhibitors include IMP321 (Immutep), which is a recombinant fusion protein of a soluble portion of LAG-3 and Ig that binds to MHC class II molecules and activates antigen presenting cells (APC). Other antibodies are described, e.g., in WO2010/019570 and US 2015/0259420.

T-cell immunoglobulin domain and mucin domain-3 (TIM-3), initially identified on activated Th1 cells, has been shown to be a negative regulator of the immune response. Blockade of TIM-3 promotes T-cell mediated anti-tumor immunity and has anti-tumor activity in a range of mouse tumor models. Combinations of TIM-3 blockade with other immunotherapeutic agents such as TSR-042, anti-CD137 antibodies and others, can be additive or synergistic in increasing anti-tumor effects. TIM-3 expression has been associated with a number of different tumor types including melanoma, NSCLC and renal cancer, and additionally, expression of intratumoral TIM-3 has been shown to correlate with poor prognosis across a range of tumor types including NSCLC, cervical, and gastric cancers. Blockade of TIM-3 is also of interest in promoting increased immunity to a number of chronic viral diseases. TIM-3 has also been shown to interact with a number of ligands including galectin-9, phosphatidylserine and HMGB1, although which of these, if any, are relevant in regulation of anti-tumor responses is not clear at present. In some embodiments, antibodies, antibody fragments, small molecules, or peptide inhibitors that target TIM-3 can bind to the IgV domain of TIM-3 to inhibit interaction with its ligands. Exemplary antibodies and peptides that inhibit TIM-3 are described in US 2015/0218274, WO2013/006490 and US 2010/0247521. Other anti-TIM-3 antibodies include humanized versions of RMT3-23 (Ngiow et al., 2011, Cancer Res, 71:3540-3551), and clone 8B.2C12 (Monney et al., 2002, Nature, 415:536-541). Bi-specific antibodies that inhibit TIM-3 and PD-1 are described in US 2013/0156774.

In some embodiments, the additional agent is a CEACAM inhibitor (e.g., CEACAM-1, CEACAM-3, and/or CEACAM-5 inhibitor). In some embodiments, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In some embodiments, the anti-CEACAM antibody binds to CEACAM-5 as described in, e.g., Zheng et al. PLoS One. (2011) 6(6): e21146), or cross reacts with CEACAM-1 and CEACAM-5 as described in, e.g., WO 2013/054331 and US 2014/0271618.

4-1BB, also known as CD137, is transmembrane glycoprotein belonging to the TNFR superfamily. 4-1BB receptors are present on activated T cells and B cells and monocytes. An exemplary anti-4-1BB antibody is urelumab (BMS-663513), which has potential immunostimulatory and antineoplastic activities.

Tumor necrosis factor receptor superfamily, member 4 (TNFRSF4), also known as OX40 and CD134, is another member of the TNFR superfamily. OX40 is not constitutively expressed on resting naïve T cells and acts as a secondary co-stimulatory immune checkpoint molecule. Exemplary anti-OX40 antibodies are MEDI6469 and MOXR0916 (RG7888, Genentech).

In some embodiments, the additional agent includes a molecule that decreases the regulatory T cell (Treg) population. Methods that decrease the number of (e.g., deplete) Treg cells are known in the art and include, e.g., CD25 depletion, cyclophosphamide administration, and modulating Glucocorticoid-induced TNFR family related gene (GITR) function. GITR is a member of the TNFR superfamily that is upregulated on activated T cells, which enhances the immune system. Reducing the number of Treg cells in a subject prior to apheresis or prior to administration of engineered cells, e.g., CAR-expressing cells, can reduce the number of unwanted immune cells (e.g., Tregs) in the tumor microenvironment and reduces the subject's risk of relapse. In some embodiments, the additional agent includes a molecule targeting GITR and/or modulating GITR functions, such as a GITR agonist and/or a GITR antibody that depletes regulatory T cells (Tregs). In some embodiments, the additional agent includes cyclophosphamide. In some embodiments, the GITR binding molecule and/or molecule modulating GITR function (e.g., GITR agonist and/or Treg depleting GITR antibodies) is administered prior to the engineered cells, e.g., CAR-expressing cells. For example, in some embodiments, the GITR agonist can be administered prior to apheresis of the cells. In some embodiments, cyclophosphamide is administered to the subject prior to administration (e.g., infusion or re-infusion) of the engineered cells, e.g., CAR-expressing cells or prior to apheresis of the cells. In some embodiments, cyclophosphamide and an anti-GITR antibody are administered to the subject prior to administration (e.g., infusion or re-infusion) of the engineered cells, e.g., CAR-expressing cells or prior to apheresis of the cells.

In some embodiments, the additional agent is a GITR agonist. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No. 090505B 1, U.S. Pat. No. 8,586,023, PCT Publication Nos.: WO 2010/003118 and 2011/090754, or an anti-GITR antibody described, e.g., in U.S. Pat. No. 7,025,962, European Patent No. 1947183B 1, U.S. Pat. Nos. 7,812,135, 8,388,967, 8,591,886, European Patent No. EP 1866339, PCT Publication No. WO 2011/028683, PCT Publication No. WO 2013/039954, PCT Publication No. WO2005/007190, PCT Publication No. WO 2007/133822, PCT Publication No. WO2005/055808, PCT Publication No. WO 99/40196, PCT Publication No. WO 2001/03720, PCT Publication No. WO99/20758, PCT Publication No. WO2006/083289, PCT Publication No. WO 2005/115451, U.S. Pat. No. 7,618,632, and PCT Publication No. WO 2011/051726. An exemplary anti-GITR antibody is TRX518.

In some embodiments, the additional agent enhances tumor infiltration or transmigration of the administered cells, e.g., CAR-expressing cells. For example, in some embodiments, the additional agent stimulates CD40, such as CD40L, e.g., recombinant human CD40L. Cluster of differentiation 40 (CD40) is also a member of the TNFR superfamily. CD40 is a costimulatory protein found on antigen-presenting cells and mediates a broad variety of immune and inflammatory responses. CD40 is also expressed on some malignancies, where it promotes proliferation. Exemplary anti-CD40 antibodies are dacetuzumab (SGN-40), lucatumumab (Novartis, antagonist), SEA-CD40 (Seattle Genetics), and CP-870,893. In some embodiments, the additional agent that enhances tumor infiltration includes tyrosine kinase inhibitor sunitnib, heparanase, and/or chemokine receptors such as CCR2, CCR4, and CCR7.

In some embodiments, the additional agent includes thalidomide drugs or analogs thereof and/or derivatives thereof, such as lenalidomide, pomalidomide or apremilast. See, e.g., Bertilaccio et al., Blood (2013) 122:4171, Otahal et al., Oncoimmunology (2016) 5(4):e1115940; Fecteau et al., Blood (2014) 124(10):1637-1644 and Kuramitsu et al., Cancer Gene Therapy (2015) 22:487-495). Lenalidomide ((RS)-3-(4-Amino-1-oxo-1,3-dihydro-2H-isoindol-2-yl)piperidine-2,6-dione; also known as Revlimid) is a synthetic derivative of thalidomide, and has multiple immunomodulatory effects, including enforcement of immune synapse formation between T cell and antigen presenting cells (APCs). For example, in some cases, lenalidomide modulates T cell responses and results in increased interleukin (IL)-2 production in CD4+ and CD8+ T cells, induces the shift of T helper (Th) responses from Th2 to Th1, inhibits expansion of regulatory subset of T cells (Tregs), and improves functioning of immunological synapses in follicular lymphoma and chronic lymphocytic leukemia (CLL) (Otahal et al., Oncoimmunology (2016) 5(4):e1115940). Lenalidomide also has direct tumoricidal activity in patients with multiple myeloma (MM) and directly and indirectly modulates survival of CLL tumor cells by affecting supportive cells, such as nurse-like cells found in the microenvironment of lymphoid tissues. Lenalidomide also can enhance T-cell proliferation and interferon-γ production in response to activation of T cells via CD3 ligation or dendritic cell-mediated activation. Lenalidomide can also induce malignant B cells to express higher levels of immunostimulatory molecules such as CD80, CD86, HLA-DR, CD95, and CD40 (Fecteau et al., Blood (2014) 124(10):1637-1644). In some embodiments, lenalidomide is administered at a dosage of from about 1 mg to about 20 mg daily, e.g., from about 1 mg to about 10 mg, from about 2.5 mg to about 7.5 mg, from about 5 mg to about 15 mg, such as about 5 mg, 10 mg, 15 mg or 20 mg daily. In some embodiments, lenalidomide is administered at a dose of from about 10 μg/kg to 5 mg/kg, e.g., about 100 μg/kg to about 2 mg/kg, about 200 μg/kg to about 1 mg/kg, about 400 μg/kg to about 600 μg/kg, such as about 500 μg/kg. In some embodiments, rituximab is administered at a dosage of about 350-550 mg/m² (e.g., 350-375, 375-400, 400-425, 425-450, 450-475, or 475-500 mg/m²), e.g., intravenously. In some embodiments, lenalidomide is administered at a low dose.

In some embodiments, the additional agent is a B-cell inhibitor. In some embodiments, the additional agent is one or more B-cell inhibitors selected from among inhibitors of CD10, CD19, CD20, CD22, CD34, CD123, CD79a, CD79b, CD179b, FLT-3, or ROR1, or a combination thereof. In some embodiments, the B-cell inhibitor is an antibody (e.g., a mono- or bispecific antibody) or an antigen binding fragment thereof. In some embodiments, the additional agent is an engineered cell expressing recombinant receptors that target B-cell targets, e.g., CD10, CD19, CD20, CD22, CD34, CD123, CD79a, CD79b, CD179b, FLT-3, or ROR1.

In some embodiments, the additional agent is a CD20 inhibitor, e.g., an anti-CD20 antibody (e.g., an anti-CD20 mono- or bi-specific antibody) or a fragment thereof. Exemplary anti-CD20 antibodies include but are not limited to rituximab, ofatumumab, ocrelizumab (also known as GA101 or RO5072759), veltuzumab, obinutuzumab, TRU-015 (Trubion Pharmaceuticals), ocaratuzumab (also known as AME-133v or ocaratuzumab), and Pro131921 (Genentech). See, e.g., Lim et al. Haematologica. (2010) 95(1):135-43. In some embodiments, the anti-CD20 antibody comprises rituximab. Rituximab is a chimeric mouse/human monoclonal antibody IgG1 kappa that binds to CD20 and causes cytolysis of a CD20 expressing cell. In some embodiments, the additional agent includes rituximab. In some embodiments, the CD20 inhibitor is a small molecule.

In some embodiments, the additional agent is a CD22 inhibitor, e.g., an anti-CD22 antibody (e.g., an anti-CD22 mono- or bi-specific antibody) or a fragment thereof. Exemplary anti-CD22 antibodies include epratuzumab and RFB4. In some embodiments, the CD22 inhibitor is a small molecule. In some embodiments, the antibody is a monospecific antibody, optionally conjugated to a second agent such as a chemotherapeutic agent. For instance, in some embodiments, the antibody is an anti-CD22 monoclonal antibody-MMAE conjugate (e.g., DCDT2980S). In some embodiments, the antibody is an scFv of an anti-CD22 antibody, e.g., an scFv of antibody RFB4. In some embodiments, the scFv is fused to all of or a fragment of Pseudomonas exotoxin-A (e.g., BL22). In some embodiments, the scFv is fused to all of or a fragment of (e.g., a 38 kDa fragment of) Pseudomonas exotoxin-A (e.g., moxetumomab pasudotox). In some embodiments, the anti-CD22 antibody is an anti-CD19/CD22 bispecific antibody, optionally conjugated to a toxin. For instance, in some embodiments, the anti-CD22 antibody comprises an anti-CD19/CD22 bispecific portion, (e.g., two scFv ligands, recognizing human CD19 and CD22) optionally linked to all of or a portion of diphtheria toxin (DT), e.g., first 389 amino acids of diphtheria toxin (DT), DT 390, e.g., a ligand-directed toxin such as DT2219ARL). In some embodiments, the bispecific portion (e.g., anti-CD 19/anti-CD22) is linked to a toxin such as deglycosylated ricin A chain (e.g., Combotox).

In some embodiments, the immunomodulatory agent is a cytokine. In some embodiments, the immunomodulatory agent is a cytokine or is an agent that induces increased expression of a cytokine in the tumor microenvironment. Cytokines have important functions related to T cell expansion, differentiation, survival, and homeostasis. Cytokines that can be administered to the subject receiving the GPRC5D-binding recombinant receptors, cells and/or compositions provided herein include one or more of IL-2, IL-4, IL-7, IL-9, IL-15, IL-18, and IL-21. Cytokines that can be administered to the subject receiving the GPRC5D-binding recombinant receptors, BCMA-binding recombinant receptors, GPRC5D- and BCMA-binding recombinant receptors, cells and/or compositions provided herein include one or more of IL-2, IL-4, IL-7, IL-9, IL-15, IL-18, and IL-21. In some embodiments, the cytokine administered is IL-7, IL-15, or IL-21, or a combination thereof. In some embodiments, administration of the cytokine to the may improve certain aspects such as response or anti-tumor activity of the administered cells, e.g., CAR-expressing cells.

Cytokine may refer to proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immunomodulatory agent is a cytokine and the cytokine is IL-4, TNF-α, GM-CSF or IL-2.

In some embodiments, the additional agent includes an interleukin-15 (IL-15) polypeptide, an interleukin-15 receptor alpha (IL-15Rα) polypeptide, or combination thereof, e.g., hetIL-15 (Admune Therapeutics, LLC). hetIL-15 is a heterodimeric non-covalent complex of IL-15 and IL-15Rα. hetIL-15 is described in, e.g., U.S. Pat. No. 8,124,084, U.S. 2012/0177598, U.S. 2009/0082299, U.S. 2012/0141413, and U.S. 2011/0081311. In some embodiments, the immunomodulatory agent can contain one or more cytokines. For example, the interleukin can include leukocyte interleukin injection (Multikine), which is a combination of natural cytokines. In some embodiments, the immunomodulatory agent is a Toll-like receptor (TLR) agonist, an adjuvant or a cytokine.

In some embodiments, the additional agent is an agent that ameliorates or neutralizes one or more toxicities or side effects associated with the cell therapy. In some embodiments, the additional agent is selected from among a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept Small molecule inhibitors of TNFα include, but are not limited to, xanthine derivatives (e.g. pentoxifylline) and bupropion. An example of an IL-6 inhibitor is an anti-IL-6 antibody molecule such as tocilizumab, sarilumab, elsilimomab, CNTO 328, ALD518/BMS-945429, CNTO 136, CPSI-2364, CDP6038, VX30, ARGX-109, FE301, and FM101. In some embodiments, the anti-IL-6 antibody molecule is tocilizumab. In some embodiments, the additional agent is an IL-1R inhibitor, such as anakinra.

In some embodiments, the additional agent is a modulator of adenosine levels and/or an adenosine pathway component. Adenosine can function as an immunomodulatory agent in the body. For example, adenosine and some adenosine analogs that non-selectively activate adenosine receptor subtypes decrease neutrophil production of inflammatory oxidative products (Cronstein et al., Ann. N.Y. Acad. Sci. 451:291, 1985; Roberts et al., Biochem. J., 227:669, 1985; Schrier et al., J. Immunol. 137:3284, 1986; Cronstein et al., Clinical Immunol. Immunopath. 42:76, 1987). In some cases, concentration of extracellular adenosine or adenosine analogs can increase in specific environments, e.g., tumor microenvironment (TME). In some cases, adenosine or adenosine analog signaling depends on hypoxia or factors involved in hypoxia or its regulation, e.g., hypoxia inducible factor (HIF). In some embodiments, increase in adenosine signaling can increase in intracellular cAMP and cAMP-dependent protein kinase that results in inhibition of proinflammatory cytokine production, and can lead to the synthesis of immunosuppressive molecules and development of Tregs (Sitkovsky et al., Cancer Immunol Res (2014) 2(7):598-605). In some embodiments, the additional agent can reduce or reverse immunosuppressive effects of adenosine, adenosine analogs and/or adenosine signaling. In some embodiments, the additional agent can reduce or reverse hypoxia-driven A2-adenosinergic T cell immunosuppression. In some embodiments, the additional agent is selected from among antagonists of adenosine receptors, extracellular adenosine-degrading agents, inhibitors of adenosine generation by CD39/CD73 ectoenzymes, and inhibitors of hypoxia-HIF-1α signaling. In some embodiments, the additional agent is an adenosine receptor antagonist or agonist.

Inhibition or reduction of extracellular adenosine or the adenosine receptor by virtue of an inhibitor of extracellular adenosine (such as an agent that prevents the formation of, degrades, renders inactive, and/or decreases extracellular adenosine), and/or an adenosine receptor inhibitor (such as an adenosine receptor antagonist) can enhance immune response, such as a macrophage, neutrophil, granulocyte, dendritic cell, T- and/or B cell-mediated response. In addition, inhibitors of the Gs protein mediated cAMP dependent intracellular pathway and inhibitors of the adenosine receptor-triggered Gi protein mediated intracellular pathways, can also increase acute and chronic inflammation.

In some embodiments, the additional agent is an adenosine receptor antagonist or agonist, e.g., an antagonist or agonist of one or more of the adenosine receptors A2a, A2b, A1, and A3. A1 and A3 inhibit, and A2a and A2b stimulate, respectively, adenylate cyclase activity. Certain adenosine receptors, such as A2a, A2b, and A3, can suppress or reduce the immune response during inflammation. Thus, antagonizing immunosuppressive adenosine receptors can augment, boost or enhance immune response, e.g., immune response from administered cells, e.g., CAR-expressing T cells. In some embodiments, the additional agent inhibits the production of extracellular adenosine and adenosine-triggered signaling through adenosine receptors. For example, enhancement of an immune response, local tissue inflammation, and targeted tissue destruction can be enhanced by inhibiting or reducing the adenosine-producing local tissue hypoxia; by degrading (or rendering inactive) accumulated extracellular adenosine; by preventing or decreasing expression of adenosine receptors on immune cells; and/or by inhibiting/antagonizing signaling by adenosine ligands through adenosine receptors.

An antagonist is any substance that tends to nullify the action of another, as an agent that binds to a cell receptor without eliciting a biological response. In some embodiments, the antagonist is a chemical compound that is an antagonist for an adenosine receptor, such as the A2a, A2b, or A3 receptor. In some embodiments, the antagonist is a peptide, or a pepidomimetic, that binds the adenosine receptor but does not trigger a Gi protein dependent intracellular pathway. Exemplary antagonists are described in U.S. Pat. Nos. 5,565,566; 5,545,627, 5,981,524; 5,861,405; 6,066,642; 6,326,390; 5,670,501; 6,117,998; 6,232,297; 5,786,360; 5,424,297; 6,313,131, 5,504,090; and 6,322,771.

In some embodiments, the additional agent is an A2 receptor (A2R) antagonist, such as an A2a antagonist. Exemplary A2R antagonists include KW6002 (istradefyline), SCH58261, caffeine, paraxanthine, 3,7-dimethyl-1-propargylxanthine (DMPX), 8-(m-chlorostyryl) caffeine (CSC), MSX-2, MSX-3, MSX-4, CGS-15943, ZM-241385, SCH-442416, preladenant, vipadenant (BII014), V2006, ST-1535, SYN-115, PSB-1115, ZM241365, FSPTP, and an inhibitory nucleic acid targeting A2R expression, e.g., siRNA or shRNA, or any antibodies or antigen-binding fragment thereof that targets an A2R. In some embodiments, the additional agent is an A2R antagonist described in, e.g., Ohta et al., Proc Natl Acad Sci USA (2006) 103:13132-13137; Jin et al., Cancer Res. (2010) 70(6):2245-2255; Leone et al., Computational and Structural Biotechnology Journal (2015) 13:265-272; Beavis et al., Proc Natl Acad Sci USA (2013) 110:14711-14716; and Pinna, A., Expert Opin Investig Drugs (2009) 18:1619-1631; Sitkovsky et al., Cancer Immunol Res (2014) 2(7):598-605; U.S. Pat. Nos. 8,080,554; 8,716,301; US 20140056922; WO2008/147482; U.S. Pat. No. 8,883,500; US 20140377240; WO02/055083; U.S. Pat. Nos. 7,141,575; 7,405,219; 8,883,500; 8,450,329 and 8,987,279).

In some embodiments, the antagonist is an antisense molecule, inhibitory nucleic acid molecule (e.g., small inhibitory RNA (siRNA)) or catalytic nucleic acid molecule (e.g. a ribozyme) that specifically binds mRNA encoding an adenosine receptor. In some embodiments, the antisense molecule, inhibitory nucleic acid molecule or catalytic nucleic acid molecule binds nucleic acids encoding A2a, A2b, or A3. In some embodiments, an antisense molecule, inhibitory nucleic acid molecule or catalytic nucleic acid targets biochemical pathways downstream of the adenosine receptor. For example, the antisense molecule or catalytic nucleic acid can inhibit an enzyme involved in the Gs protein- or Gi protein-dependent intracellular pathway. In some embodiments, the additional agent includes dominant negative mutant form of an adenosine receptor, such as A2a, A2b, or A3.

In some embodiments, the additional agent that inhibits extracellular adenosine includes agents that render extracellular adenosine non-functional (or decrease such function), such as a substance that modifies the structure of adenosine to inhibit the ability of adenosine to signal through adenosine receptors. In some embodiments, the additional agent is an extracellular adenosine-generating or adenosine-degrading enzyme, a modified form thereof or a modulator thereof. For example, in some embodiments, the additional agent is an enzyme (e.g. adenosine deaminase) or another catalytic molecule that selectively binds and destroys the adenosine, thereby abolishing or significantly decreasing the ability of endogenously formed adenosine to signal through adenosine receptors and terminate inflammation.

In some embodiments, the additional agent is an adenosine deaminase (ADA) or a modified form thereof, e.g., recombinant ADA and/or polyethylene glycol-modified ADA (ADA-PEG), which can inhibit local tissue accumulation of extracellular adenosine. ADA-PEG has been used in treatment of patients with ADA SCID (Hershfield (1995) Hum Mutat. 5:107). In some embodiments, an agent that inhibits extracellular adenosine includes agents that prevent or decrease formation of extracellular adenosine, and/or prevent or decrease the accumulation of extracellular adenosine, thereby abolishing, or substantially decreasing, the immunosuppressive effects of adenosine. In some embodiments, the additional agent specifically inhibits enzymes and proteins that are involved in regulation of synthesis and/or secretion of pro-inflammatory molecules, including modulators of nuclear transcription factors. Suppression of adenosine receptor expression or expression of the Gs protein- or Gi protein-dependent intracellular pathway, or the cAMP dependent intracellular pathway, can result in an increase/enhancement of immune response.

In some embodiments, the additional agent can target ectoenzymes that generate or produce extracellular adenosine. In some embodiments, the additional agent targets CD39 and CD73 ectoenzymes, which function in tandem to generate extracellular adenosine. CD39 (also called ectonucleoside triphosphate diphosphohydrolase) converts extracellular ATP (or ADP) to 5′AMP. Subsequently, CD73 (also called 5′nucleotidase) converts 5′AMP to adenosine. The activity of CD39 is reversible by the actions of NDP kinase and adenylate kinase, whereas the activity of CD73 is irreversible. CD39 and CD73 are expressed on tumor stromal cells, including endothelial cells and Tregs, and also on many cancer cells. For example, the expression of CD39 and CD73 on endothelial cells is increased under the hypoxic conditions of the tumor microenvironment. Tumor hypoxia can result from inadequate blood supply and disorganized tumor vasculature, impairing delivery of oxygen (Carroll and Ashcroft (2005), Expert. Rev. Mol. Med. 7(6):1-16). Hypoxia also inhibits adenylate kinase (AK), which converts adenosine to AMP, leading to very high extracellular adenosine concentration. Thus, adenosine is released at high concentrations in response to hypoxia, which is a condition that frequently occurs the tumor microenvironment (TME), in or around solid tumors. In some embodiments, the additional agent is one or more of anti-CD39 antibody or antigen binding fragment thereof, anti-CD73 antibody or antigen binding fragment thereof, e.g., MEDI9447 or TY/23, α-β-methylene-adenosine diphosphate (ADP), ARL 67156, POM-3, IPH52 (see, e.g., Allard et al. Clin Cancer Res (2013) 19(20):5626-5635; Hausler et al., Am J Transl Res (2014) 6(2):129-139; Zhang, B., Cancer Res. (2010) 70(16):6407-6411).

In some embodiments, the additional agent is an inhibitor of hypoxia inducible factor 1 alpha (HIF-1α) signaling. Exemplary inhibitors of HIF-1α include digoxin, acriflavine, sirtuin-7 and ganetespib.

In some embodiments, the additional agent includes a protein tyrosine phosphatase inhibitor, e.g., a protein tyrosine phosphatase inhibitor described herein. In some embodiments, the protein tyrosine phosphatase inhibitor is an SHP-1 inhibitor, e.g., an SHP-1 inhibitor described herein, such as, e.g., sodium stibogluconate. In some embodiments, the protein tyrosine phosphatase inhibitor is an SHP-2 inhibitor, e.g., an SHP-2 inhibitor described herein.

In some embodiments, the additional agent is a kinase inhibitor. Kinase inhibitors, such as a CDK4 kinase inhibitor, a BTK kinase inhibitor, a MNK kinase inhibitor, or a DGK kinase inhibitor, can regulate the constitutively active survival pathways that exist in tumor cells and/or modulate the function of immune cells. In some embodiments, the kinase inhibitor is a Bruton's tyrosine kinase (BTK) inhibitor, e.g., ibrutinib. In some embodiments, the kinase inhibitor is a phosphatidylinositol-4,5-bisphosphate 3-kinase (PI3K) inhibitor. In some embodiments, the kinase inhibitor is a CDK4 inhibitor, e.g., a CDK4/6 inhibitor. In some embodiments, the kinase inhibitor is an mTOR inhibitor, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor. In some embodiments, the kinase inhibitor is an MNK inhibitor, or a dual PI3K/mTOR inhibitor. In some embodiments, other exemplary kinase inhibitors include the AKT inhibitor perifosine, the mTOR inhibitor temsirolimus, the Src kinase inhibitors dasatinib and fostamatinib, the JAK2 inhibitors pacritinib and ruxolitinib, the PKCβ inhibitors enzastaurin and bryostatin, and the AAK inhibitor alisertib.

In some embodiments, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In some embodiments, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In some embodiments, the kinase inhibitor is a BTK inhibitor, e.g., ibrutinib (1-[(3R)-3-[4-Amino-3-(4-phenoxyphenyl)-1H-pyrazolo[3,4-d]pyrimidin-1-yl]piperidin-1-yl]prop-2-en-1-one; also known as PCI-32765). In some embodiments, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In some embodiments, 1, 2, 3, 4, 5, 6, 7, 8, 9, 10, 11, 12 or more cycles of ibrutinib are administered. In some embodiments, the BTK inhibitor is a BTK inhibitor described in International Application WO 2015/079417.

In some embodiments, the kinase inhibitor is a PI3K inhibitor. PI3K is central to the PI3K/Akt/mTOR pathway involved in cell cycle regulation and lymphoma survival. Exemplary PI3K inhibitor includes idelalisib (PI3Kδ inhibitor). In some embodiments, the additional agent is idelalisib and rituximab.

In some embodiments, the additional agent is an inhibitor of mammalian target of rapamycin (mTOR). In some embodiments, the kinase inhibitor is an mTOR inhibitor selected from temsirolimus; ridaforolimus (also known as AP23573 and MK8669); everolimus (RAD001); rapamycin (AY22989); simapimod; AZD8055; PF04691502; SF1126; and XL765. In some embodiments, the additional agent is an inhibitor of mitogen-activated protein kinase (MAPK), such as vemurafenib, dabrafenib, and trametinib.

In some embodiments, the additional agent is an agent that regulates pro- or anti-apoptotic proteins. In some embodiments, the additional agent includes a B-cell lymphoma 2 (BCL-2) inhibitor (e.g., venetoclax, also called ABT-199 or GDC-0199; or ABT-737). Venetoclax is a small molecule (4-(4-{[2-(4-Chlorophenyl)-4,4-dimethyl-1-cyclohexen-1-yl]methyl}-1-piperazinyl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) that inhibits the anti-apoptotic protein, BCL-2. Other agents that modulate pro- or anti-apoptotic protein include BCL-2 inhibitor ABT-737, navitoclax (ABT-263); Mc1-1 siRNA or Mc1-1 inhibitor retinoid N-(4-hydroxyphenyl) retinamide (4-HPR) for maximal efficacy. In some embodiments, the additional agent provides a pro-apoptotic stimuli, such as recombinant tumor necrosis factor-related apoptosis-inducing ligand (TRAIL), which can activate the apoptosis pathway by binding to TRAIL death receptors DR-4 and DR-5 on tumor cell surface, or TRAIL-R2 agonistic antibodies.

In some embodiments, the additional agent includes an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. Plasmacytoid dendritic cells (pDCs), macrophages, and dendritic cells (DCs) can express IDO. In some aspects, a decrease in L-tryptophan (e.g., catalyzed by IDO) results in an immunosuppressive milieu by inducing T-cell anergy and apoptosis. Thus, in some aspects, an IDO inhibitor can enhance the efficacy of the GPRC5D-binding recombinant receptors, cells and/or compositions described herein, e.g., by decreasing the suppression or death of the administered CAR-expressing cell. Exemplary inhibitors of IDO include but are not limited to 1-methyl-tryptophan, indoximod (New Link Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255).

In some embodiments, the additional agent includes a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. In some embodiments, the additional agent includes a hypomethylating agent, e.g., a DNA methyltransferase inhibitor, e.g., azacitidine or decitabine.

In another embodiment, the additional therapy is transplantation, e.g., an allogeneic stem cell transplant.

In some embodiments, the additional therapy is a lymphodepleting therapy. Lymphodepleting chemotherapy is thought to improve engraftment and activity of recombinant receptor-expressing cells, such as CAR T cells. In some embodiments, lymphodepleting chemotherapy may enhance adoptively transferred tumor-specific T cells to proliferate in vivo through homeostatic proliferation (Grossman 2004, Stachel 2004). In some embodiments, chemotherapy may reduce or eliminate CD4+CD25+ regulatory T cells, which can suppress the function of tumor-targeted adoptively transferred T cells (Turk 2004). In some embodiments, lymphodepleting chemotherapy prior to adoptive T-cell therapy may enhance the expression of stromal cell-derived factor 1 (SDF-1) in the bone marrow, enhancing the homing of modified T cells to the primary tumor site through binding of SDF-1 with CXCR-4 expressed on the T-cell surface (Pinthus 2004). In some embodiments, lymphodepleting chemotherapy may further reduce the subject's tumor burden and potentially lower the risk and severity of CRS.

In some embodiments, lymphodepletion is performed on a subject, e.g., prior to administering engineered cells, e.g., CAR-expressing cells. In some embodiments, the lymphodepletion comprises administering one or more of melphalan, Cytoxan, cyclophosphamide, and/or fludarabine. In some embodiments, a lymphodepleting chemotherapy is administered to the subject prior to, concurrently with, or after administration (e.g., infusion) of engineered cells, e.g., CAR-expressing cells. In an example, the lymphodepleting chemotherapy is administered to the subject prior to administration of engineered cells, e.g., CAR-expressing cells. In some embodiments the lymphodepleting chemotherapy is administered 1 to 10 days prior to administration of engineered cells, such as 1, 2, 3, 4, 5, 6, 7, 8, 9, or 10 days prior to the initiation of administration of engineered cells, or at least 2 days prior, such as at least 3, 4, 5, 6, or 7 days prior, to the initiation of administration of engineered cell. In some embodiments, the subject is administered a preconditioning agent no more than 7 days prior, such as no more than 6, 5, 4, 3, or 2 days prior, to the initiation of administration of engineered cell. The number of days after lymphodepleting chemotherapy that the engineered ells are administered can be determined based on clinical or logistical circumstances. In some examples, dose adjustments or other changes to the lymphodepleting chemotherapy regimen can implemented due to a subject's health, such as the subject's underlying organ function, as determined by the treating physician.

In some embodiments, lymphodepleting chemotherapy comprises administration of a lymphodepleting agent, such as cyclophosphamide, fludarabine, or combinations thereof. In some embodiments, the subject is administered cyclophosphamide at a dose between or between about 20 mg/kg and 100 mg/kg body weight of the subject, such as between or between about 40 mg/kg and 80 mg/kg. In some aspects, the subject is administered about 60 mg/kg of cyclophosphamide. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m² body surface area of the subject, such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 2 to 4 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m² body surface area of the subject, such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 2 to 4 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered fludarabine at or about 30 mg/m², daily, and cyclophosphamide at or about 300 mg/m², daily, for 3 days.

In some embodiments, antiemetic therapy, except dexamethasone or other steroids, may be given prior to lymphodepleting chemotherapy. In some embodiments, Mesna may be used for subjects with a history of hemorrhagic cystitis.

In some embodiments, the additional agent is an oncolytic virus. In some embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

Other exemplary combination therapy, treatment and/or agents include anti-allergenic agents, anti-emetics, analgesics and adjunct therapies. In some embodiments, the additional agent includes cytoprotective agents, such as neuroprotectants, free-radical scavengers, cardioprotectors, anthracycline extravasation neutralizers and nutrients.

In some embodiments, an antibody used as an additional agent is conjugated or otherwise bound to a therapeutic agent, e.g., a chemotherapeutic agent (e.g., Cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein. In some embodiments, the additional agent is an antibody-drug conjugate.

In some embodiments, the additional agent can modulate, inhibit or stimulate particular factors at the DNA, RNA or protein levels, such as to enhance or boost certain aspects. In some embodiments, the additional agent can modulate the factors at the nucleic acid level, e.g., DNA or RNA, within the administered cells, e.g., cells engineered to express recombinant receptors, e.g., CAR. In some embodiments, an inhibitory nucleic acid, e.g., an inhibitory nucleic acid, e.g., a dsRNA, e.g., an siRNA or shRNA, or a clustered regularly interspaced short palindromic repeats (CRISPR), a transcription-activator like effector nuclease (TALEN), or a zinc finger endonuclease (ZFN), can be used to inhibit expression of an inhibitory molecule in the engineered cell, e.g., CAR-expressing cell. In some embodiments the inhibitor is an shRNA. In some embodiments, the inhibitory molecule is inhibited within the engineered cell, e.g., CAR-expressing cell. In some embodiments, a nucleic acid molecule that encodes a dsRNA molecule that inhibits expression of the molecule that modulates or regulates, e.g., inhibits, T-cell function is operably linked to a promoter, e.g., a HI- or a U6-derived promoter such that the dsRNA molecule that inhibits expression of the inhibitory molecule is expressed within the engineered cell, e.g., CAR-expressing cell. See, e.g., Brummelkamp T R, et al. (2002) Science 296: 550-553; Miyagishi M, et al. (2002) Nat. Biotechnol. 19: 497-500.

In some embodiments, the additional agent is capable of disrupting the gene encoding an inhibitory molecule, such as any immune checkpoint inhibitors described herein. In some embodiments, disruption is by deletion, e.g., deletion of an entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the inhibitory molecule is not expressed or is not expressed in a form that is capable of being expressed on the cells surface and/or capable of mediating cell signaling. The disruption is generally carried out at the DNA level. The disruption generally is permanent, irreversible, or not transient.

In some aspects, the disruption is carried out by gene editing, such as using a DNA binding protein or DNA-binding nucleic acid, which specifically binds to or hybridizes to the gene at a region targeted for disruption. In some aspects, the protein or nucleic acid is coupled to or complexed with a nuclease, such as in a chimeric or fusion protein. For example, in some embodiments, the disruption is effected using a fusion comprising a DNA-targeting protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)-Cas system, such as CRISPR-Cas9 system, specific for the gene being disrupted. In some embodiments, methods of producing or generating genetically engineered cells, e.g., CAR-expressing cells, include introducing into a population of cells nucleic acid molecules encoding a genetically engineered antigen receptor (e.g. CAR) and nucleic acid molecules encoding an agent targeting an inhibitory molecule that is a gene editing nuclease, such as a fusion of a DNA-targeting protein and a nuclease such as a ZFN or a TALEN, or an RNA-guided nuclease such as of the CRISPR-Cas9 system, specific for an inhibitory molecule.

Any of the additional agents described herein can be prepared and administered as combination therapy with the GPRC5D-binding recombinant receptor (e.g., chimeric antigen receptor) and/or engineered cells expressing said molecules (e.g., recombinant receptor) described herein, such as in pharmaceutical compositions comprising one or more agents of the combination therapy and a pharmaceutically acceptable carrier, such as any described herein. Any of the additional agents described herein can be prepared and administered as combination therapy with the GPRC5D-binding (and BCMA-binding) recombinant receptor (e.g., chimeric antigen receptor) and/or engineered cells expressing said molecules (e.g., recombinant receptor) described herein, such as in pharmaceutical compositions comprising one or more agents of the combination therapy and a pharmaceutically acceptable carrier, such as any described herein. In some embodiments, the GPRC5D-binding recombinant receptor (e.g., chimeric antigen receptor), engineered cells expressing said molecules (e.g., recombinant receptor), plurality of engineered cells expressing said molecules (e.g., recombinant receptor) can be administered simultaneously, concurrently or sequentially, in any order with the additional agents, therapy or treatment, wherein such administration provides therapeutically effective levels each of the agents in the body of the subject. In some embodiments, the additional agent can be co-administered with the GPRC5D-binding recombinant receptors, cells and/or compositions described herein, for example, as part of the same pharmaceutical composition or using the same method of delivery. In some embodiments, the additional agent is administered simultaneously with the GPRC5D-binding recombinant receptors, cells and/or compositions described herein, but in separate compositions. In some embodiments, the additional agent is an additional engineered cell, e.g., cell engineered to express a different recombinant receptor, and is administered in the same composition or in a separate composition. In some embodiments, the additional agent is incubated with the engineered cell, e.g., CAR-expressing cells, prior to administration of the cells.

In some examples, the one or more additional agents are administered subsequent to or prior to the administration of the GPRC5D-binding recombinant receptors, cells and/or compositions described herein, separated by a selected time period. In some examples, the one or more additional agents are administered subsequent to or prior to the administration of the GPRC5D-binding (and BCMA-binding) recombinant receptors, cells and/or compositions described herein, separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times and/or the GPRC5D-binding recombinant receptors, cells and/or compositions described herein, is administered multiple times. For example, in some embodiments, the additional agent is administered prior to the GPRC5D-binding recombinant receptors, cells and/or compositions described herein, e.g., two weeks, 12 days, 10 days, 8 days, one week, 6 days, 5 days, 4 days, 3 days, 2 days or 1 day before the administration. For example, in some embodiments, the additional agent is administered after the GPRC5D-binding recombinant receptors, cells and/or compositions described herein, e.g., two weeks, 12 days, 10 days, 8 days, one week, 6 days, 5 days, 4 days, 3 days, 2 days or 1 day after the administration.

The dose of the additional agent can be any therapeutically effective amount, e.g., any dose amount described herein, and the appropriate dosage of the additional agent may depend on the type of disease to be treated, the type, dose and/or frequency of the recombinant receptor, cell and/or composition administered, the severity and course of the disease, whether the recombinant receptor, cell and/or composition is administered for preventive or therapeutic purposes, previous therapy, the patient's clinical history and response to the recombinant receptor, cell and/or composition, and the discretion of the attending physician. The recombinant receptor, cell and/or composition and/or the additional agent and/or therapy can be administered to the patient at one time, repeated or administered over a series of treatments.

### VI. Articles of Manufacture or Kits

Also provided are articles of manufacture or kits containing the provided recombinant receptors (e.g., CARs), genetically engineered cells, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., multiple myeloma). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating the GPRC5D-expressing or GPRC5D-associated disease, disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a GPRC5D-expressing or GPRC5D-associated disease, disorder or condition in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the recombinant receptor (e.g., CAR or engineered cell containing the CAR); and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

In some embodiments, the article of manufacture or kit may include (a) a first container with a composition contained therein (i.e., first medicament), wherein the composition includes the anti-GPRC5D recombinant receptor (e.g., CAR or engineered cell containing the CAR); (b) a second container with a composition contained therein (i.e., second medicament), wherein the composition includes a second recombinant receptor (e.g., CAR or engineered cell containing the CAR) directed to a different epitope of GPRC5D or a different antigen (e.g., BCMA), and which article or kit further comprises instructions on the label or package insert for treating the subject with the second medicament, in an effective amount. In some embodiments, the article of manufacture or kit may further include (c) a third container with a with a composition contained therein (i.e., third medicament), wherein the composition includes a further agent, such as a cytotoxic or otherwise therapeutic agent, and which article or kit further comprises instructions on the label or package insert for treating the subject with the third medicament, in an effective amount.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-GPRC5D antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

### VIII. Exemplary Embodiments

Among the provided embodiments are:

1. A chimeric antigen receptor comprising:
(1) an extracellular antigen-binding domain that specifically binds human G-protein coupled receptor class C group 5 member D (GPRC5D), wherein the extracellular antigen-binding domain comprises:
   (i) a variable heavy chain (VH) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:21, 23, 25, 27, 29, 31 or 33; and
   (ii) a variable light chain (VL) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:22, 24, 26, 28, 30, 32, 34, 63, 64, 65, 66, 67, 68 or 69;
(2) a spacer of at least 125 amino acids in length;
(3) a transmembrane domain; and
(4) an intracellular signaling region.
   2. A chimeric antigen receptor comprising:
(1) an extracellular antigen-binding domain that specifically binds human G-protein coupled receptor class C group 5 member D (GPRC5D), wherein the extracellular antigen-binding domain comprises:
   (i) a variable heavy chain (VH) region comprising a heavy chain complementarity determining region 1 (CDR-H1), heavy chain complementarity determining region 2 (CDR-H2) and heavy chain complementarity determining region 3 (CDR-H3) contained within the VH region amino acid sequence selected from SEQ ID NOs: 21, 23, 25, 27, 29, 31, and 33; and
   (ii) a variable light chain (VL) region comprising a light chain complementarity determining region 1 (CDR-L1), light chain complementarity determining region 2 (CDR-L2) and light chain complementarity determining region 3 (CDR-L3) contained within the VL region amino acid sequence selected from SEQ ID NOs: 22, 24, 26, 28, 30, 32, 34, 63, 64, 65, 66, 67, 68, and 69;
(2) a spacer of at least 125 amino acids in length;
(3) a transmembrane domain; and
(4) an intracellular signaling region.
   3. A chimeric antigen receptor comprising:
(1) an extracellular antigen-binding domain that specifically binds human G-protein coupled receptor class C group 5 member D (GPRC5D), wherein the extracellular antigen-binding domain comprises:
   (i) a variable heavy chain (VH) region comprising a CDR-H1 comprising the amino acid sequence selected from SEQ ID NOs: 75, 78, 80, 82, 90, 93, 95, 97, 105, 108, 110, 112, 120, 123, 125, 127, 135, 138, 140, 142, 152, 162, 165, 167, and 169; (b) a CDR-H2 comprising the amino acid sequence selected from SEQ ID NOs: 76, 79, 81, 83, 91, 94, 96, 98, 106, 109, 111, 113, 121, 124, 126, 128, 136, 139, 141, 143, 150, 153, 154, 155, 163, 166, 168, and 170; and (c) a CDR-H3 comprising the amino acid sequence selected from SEQ ID NOs: 77, 84, 92, 99, 107, 114, 122, 129, 137, 144, 151, 156, 164, and 171; and
   (ii) a variable light chain (VL) region comprising a CDR-L1 comprising the amino acid sequence selected from SEQ ID NOs: 85, 88, 100, 103, 115, 118, 130, 133, 145, 148, 157, 160, 172, and 174; (b) a CDR-L2 comprising the amino acid sequence selected from SEQ ID NOs: 86, 89, 101, 104, 116, 119, 131, 134, 146, 149, 158, and 161; and (c) a CDR-L3 comprising the amino acid sequence selected from SEQ ID NOs: 87, 102, 117, 132, 147, 159, 173, 175, and 297;
(2) a spacer of at least 125 amino acids in length;
(3) a transmembrane domain; and
(4) an intracellular signaling region.
   4. The chimeric antigen receptor of embodiment 2 or embodiment 3, wherein the extracellular antigen-binding domain comprises:
(i) a variable heavy chain (VH) region comprising: an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:21, 23, 25, 27, 29, 31 or 33; and
(ii) a variable light chain (VL) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:22, 24, 26, 28, 30, 32, 34, 63, 64, 65, 66, 67, 68 or 69.
   5. The chimeric antigen receptor of any of embodiments 1-4, wherein the spacer has a length of between 125 and 300 or of between about 125 and about 300, of between 125 and 250 or of between about 125 and about 250, of between 125 and 230 or of between about 125 and about 230, of between 125 and 200 or of between about 125 and about 200, of between 125 and 180 or of between about 125 and about 180, of between 125 and 150 or of between about 125 and about 150, of between 150 and 300 or of between about 150 and about 300, of between 150 and 250 or of between about 150 and about 250, of between 150 and 230 or of between about 150 and about 230, of between 150 and 200 or of between about 150 and about 200, of between 150 and 180 or of between about 150 and about 180, of between 180 and 300 or of between about 180 and about 300, of between 180 and 250 or of between about 180 and about 250, of between 125 and 300 or of between about 125 and about 300, of between 180 and 230 or of between about 180 and about 230, of between 180 and 200 or of between about 180 and about 200, of between 200 and 300 or of between about 200 and about 300, of between 200 and 250 or of between about 200 and about 250, of between 200 and 230 or of between about 200 and about 230, of between 230 and 300 or of between about 230 and about 300, of between 230 and 250 or of between about 230 and about 250 or of between 250 and 300 or of between about 250 and about 300.
   6. The chimeric antigen receptor of any of embodiments 1-5, wherein:
the spacer is, or is at least about, 130, 140, 150, 160, 170, 180, 190, 200, 210, 220, 221, 222, 223, 224, 225, 226, 227, 228 or 229 amino acids in length, or has a length between any of the foregoing; or
the spacer is about, or is at least about, 130, 140, 150, 160, 170, 180, 190, 200, 210, 220, 221, 222, 223, 224, 225, 226, 227, 228 or 229 amino acids in length, or has a length between any of the foregoing.
   7. The chimeric antigen receptor of any of embodiments 1-6, wherein the spacer comprises a portion of an immunoglobulin.
   8. The chimeric antigen receptor of any of embodiments 1-7, wherein the spacer comprises a sequence of a hinge region, a CH2 and CH3 region.
   9. The chimeric antigen receptor of embodiment 8, wherein:
the hinge region comprises all or a portion of an IgG4 hinge region and/or an IgG2 hinge region, wherein the IgG4 hinge region is optionally a human IgG4 hinge region and the IgG2 hinge region is optionally a human IgG2 hinge region;
the CH2 region comprises all or a portion of an IgG4 CH2 and/or an IgG2 CH2, wherein the IgG4 CH2 is optionally a human IgG4 CH2 and the IgG2 CH2 is optionally a human IgG2 CH2; and/or
the CH3 region comprises all or a portion of an IgG4 CH3 and/or an IgG2 CH3, wherein the IgG4 CH3 is optionally a human IgG4 CH3 and the IgG2 CH3 is optionally a human IgG2 C_{H}3.
   10. The chimeric antigen receptor of embodiment 8 or embodiment 9, wherein the hinge region, CH2 and CH3 comprises all or a portion of a hinge, all or a portion of a CH2 and all or a portion of a CH3 from human IgG4.
   11. The chimeric antigen receptor of embodiment 8 or embodiment 9, wherein one or more of the hinge region, the CH2 and the CH3 is chimeric and comprises a hinge, CH2 and CH3 from human IgG4 and human IgG2.
   12. The chimeric antigen receptor of any of embodiments 1-11, wherein the spacer comprises a IgG4/2 chimeric hinge region or a modified IgG4 hinge region comprising at least one amino acid replacement compared to a human IgG4 hinge; an IgG2/4 chimeric CH2 region; and an IgG4 CH3 region.
   13. The chimeric antigen receptor of any of embodiments 1-12, wherein the spacer is or comprises (i) the sequence set forth in SEQ ID NO: 17; (ii) a functional variant of SEQ ID NO:17 that has at least at or about 95%, at or about 96%, at or about 97%, at or about 98% or at or about 99% sequence identity to SEQ ID NO:17; or (iii) a contiguous portion of (i) or (ii) that is at least 125 amino acids in length.
   14. The chimeric antigen receptor of any of embodiments 1-13, wherein the spacer is or comprises the sequence set forth in SEQ ID NO:17.
   15. The chimeric antigen receptor of any of embodiments 1-14, wherein the spacer is or comprises the amino acid sequence encoded by the nucleotide sequence set forth in SEQ ID NO: 48.
   16. The chimeric antigen receptor of any of embodiments 1-15, wherein:
the VH region comprises the amino acid sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and the VL region comprises the amino acid sequence set forth in SEQ ID NO:22 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:22;
   the VH region comprises the amino acid sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and the VL region comprises the amino acid sequence set forth in SEQ ID NO:63 or or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:63;
   the VH region comprises the amino acid sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and the VL region comprises the amino acid sequence set forth in SEQ ID NO:24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24;
   the VH region comprises the amino acid sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and the VL region comprises the amino acid sequence set forth in SEQ ID NO:64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:64;
   the VH region comprises the amino acid sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25; and the VL region comprises the amino acid sequence set forth in SEQ ID NO:26 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:26;
   the VH region comprises the amino acid sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25; and the VL region comprises the amino acid sequence set forth in SEQ ID NO:65 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:65;
   the VH region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the VL region comprises the amino acid sequence set forth in SEQ ID NO:28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:28;
   the VH region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the VL region comprises the amino acid sequence set forth in SEQ ID NO:66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:66;
   the VH region comprises the amino acid sequence set forth in SEQ ID NO:29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:29; and the VL region comprises the amino acid sequence set forth in SEQ ID NO:30 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:30;
   the VH region comprises the amino acid sequence set forth in SEQ ID NO:29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:29; and the VL region comprises the amino acid sequence set forth in SEQ ID NO:67 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:67;
   the VH region comprises the amino acid sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and the VL region comprises the amino acid sequence set forth in SEQ ID NO:32 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:32;
   the VH region comprises the amino acid sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and the VL region comprises the amino acid sequence set forth in SEQ ID NO:68 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:68;
   the VH region comprises the amino acid sequence set forth in SEQ ID NO:33 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:33; and the VL region comprises the amino acid sequence set forth in SEQ ID NO:34 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:34; or
   the VH region comprises the amino acid sequence set forth in SEQ ID NO:33 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:33; and the VL region comprises the amino acid sequence set forth in SEQ ID NO:69 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:69.
      17. The chimeric antigen receptor of any of embodiments 1-16, wherein:
the VH region comprises a CDR-H1, CDR-H2, and CDR-H3 comprising the amino acid sequences of SEQ ID NOS:80, 81 and 77, respectively, and the VL region comprises a CDR-L1, CDR-L2, and CDR-L3 comprising the amino acid sequences of SEQ ID NOS:85, 86 and 87, respectively;
the VH region comprises a CDR-H1, CDR-H2, and CDR-H3 comprising the amino acid sequences of SEQ ID NOS:82, 83 and 84, respectively, and the VL region comprises a CDR-L1, CDR-L2, and CDR-L3 comprising the amino acid sequences of SEQ ID NOS:88, 89 and 87, respectively;
the VH region comprises a CDR-H1, CDR-H2, and CDR-H3 comprising the amino acid sequences of SEQ ID NOS:95, 96, 92, respectively, and the VL region comprises a CDR-L1, CDR-L2, and CDR-L3 comprising the amino acid sequences of SEQ ID NOS:100, 101 and 102, respectively;
the VH region comprises a CDR-H1, CDR-H2, and CDR-H3 comprising the amino acid sequences of SEQ ID NOS:97, 98 and 99, respectively, and the VL region comprises a CDR-L1, CDR-L2, and CDR-L3 comprising the amino acid sequences of SEQ ID NOS:103, 104 and 102, respectively;
the VH region comprises a CDR-H1, CDR-H2, and CDR-H3 comprising the amino acid sequences of SEQ ID NOS:110, 111 and 107, respectively, and the VL region comprises a CDR-L1, CDR-L2, and CDR-L3 comprising the amino acid sequences of SEQ ID NOS:115, 116 and 117, respectively;
the VH region comprises a CDR-H1, CDR-H2, and CDR-H3 comprising the amino acid sequences of SEQ ID NOS:112, 113 and 114, respectively, and the VL region comprises a CDR-L1, CDR-L2, and CDR-L3 comprising the amino acid sequences of SEQ ID NOS:118, 119 and 117, respectively;
the VH region comprises a CDR-H1, CDR-H2, and CDR-H3 comprising the amino acid sequences of SEQ ID NOS:125, 126 and 122, respectively, and the VL region comprises a CDR-L1, CDR-L2, and CDR-L3 comprising the amino acid sequences of SEQ ID NOS:130, 131 and 132, respectively;
the VH region comprises a CDR-H1, CDR-H2, and CDR-H3 comprising the amino acid sequences of SEQ ID NOS:127, 128 and 129, respectively, and the VL region comprises a CDR-L1, CDR-L2, and CDR-L3 comprising the amino acid sequences of SEQ ID NOS:133, 134 and 132, respectively;
the VH region comprises a CDR-H1, CDR-H2, and CDR-H3 comprising the amino acid sequences of SEQ ID NOS:140, 141 and 137, respectively, and the VL region comprises the amino acid sequence of SEQ ID NOS:145, 146 and 147, respectively;
the VH region comprises a CDR-H1, CDR-H2, and CDR-H3 comprising the amino acid sequences of SEQ ID NOS:142, 143 and 144, respectively, and the VL region comprises a CDR-L1, CDR-L2, and CDR-L3 comprising the amino acid sequences of SEQ ID NOS:148, 149 and 147, respectively;
the VH region comprises a CDR-H1, CDR-H2, and CDR-H3 comprising the amino acid sequences of SEQ ID NOS:140, 154 and 151, respectively, and the VL region comprises a CDR-L1, CDR-L2, and CDR-L3 comprising the amino acid sequences of SEQ ID NOS:157, 158 and 159, respectively;
the VH region comprises a CDR-H1, CDR-H2, and CDR-H3 comprising the amino acid sequences of SEQ ID NOS:142, 155 and 156, respectively, and the VL region comprises a CDR-L1, CDR-L2, and CDR-L3 comprising the amino acid sequences of SEQ ID NOS:160, 161 and 159, respectively;
the VH region comprises a CDR-H1, CDR-H2, and CDR-H3 comprising the amino acid sequences of SEQ ID NOS:167, 168 and 164, respectively, and the VL region comprises a CDR-L1, CDR-L2, and CDR-L3 comprising the amino acid sequences of SEQ ID NOS:172, 86, 173, respectively;
the VH region comprises a CDR-H1, CDR-H2, and CDR-H3 comprising the amino acid sequences of SEQ ID NOS:169, 170 and 171, respectively, and the VL region comprises a CDR-L1, CDR-L2, and CDR-L3 comprising the amino acid sequences of SEQ ID NOS:174, 89 and 175, respectively; or
the VH region comprises a CDR-H1, CDR-H2, and CDR-H3 comprising the amino acid sequences of SEQ ID NOS:169, 170 and 171, respectively, and the VL region comprises a CDR-L1, CDR-L2, and CDR-L3 comprising the amino acid sequences of SEQ ID NOS:174, 89 and 297, respectively.
   18. The chimeric antigen receptor of any of embodiments 1-17, wherein:
the VH region and the VL region comprise the amino acid sequence set forth in SEQ ID NOs:21 and 22, respectively or the amino acid sequence set forth in SEQ ID NOs: 21 and 63, respectively;
the VH region and the VL region comprise the amino acid sequence set forth in SEQ ID NOs:23 and 24, respectively or the amino acid sequence set forth in SEQ ID NOs:23 and 64, respectively;
the VH region and the VL region comprise the amino acid sequence set forth in SEQ ID NOs:25 and 26, respectively or the amino acid sequence set forth in SEQ ID NOs: 25 and 65, respectively;
the VH region and the VL region comprise the amino acid sequence set forth in SEQ ID NOs:27 and 28, respectively or the amino acid sequence set forth in SEQ ID NOs: 27 and 66, respectively;
the VH region and the VL region comprise the amino acid sequence set forth in SEQ ID NOs:29 and 30, respectively or the amino acid sequence set forth in SEQ ID NOs:29 and 67, respective;
the VH region and the VL region comprise the amino acid sequence set forth in SEQ ID NOs:31 and 32, respectively or the amino acid sequence set forth in SEQ ID Nos: 31 and 68, respectively; or
the VH region and the VL region comprise the amino acid sequence set forth in SEQ ID NOs:33 and 34 or the amino acid sequence set forth in SEQ ID Nos: 33 and 69, respectively, respectively.
   19. The chimeric antigen receptor of any of embodiments 1-18, wherein:
the VH region comprises the amino acid sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and the VL region comprises the amino acid sequence set forth in SEQ ID NO:22 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:22;
   the VH region comprises the amino acid sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and the VL region comprises the amino acid sequence set forth in SEQ ID NO:63 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:63;
the VH region comprises the amino acid sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and the VL region comprises the amino acid sequence set forth in SEQ ID NO:24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24;
   the VH region comprises the amino acid sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and the VL region comprises the amino acid sequence set forth in SEQ ID NO:64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:64;
the VH region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the VL region comprises the amino acid sequence set forth in SEQ ID NO:28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:28;
   the VH region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the VL region comprises the amino acid sequence set forth in SEQ ID NO:66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:66;
the VH region comprises the amino acid sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and the VL region comprises the amino acid sequence set forth in SEQ ID NO:32 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:32; or
   the VH region comprises the amino acid sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and the VL region comprises the amino acid sequence set forth in SEQ ID NO:68 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:68.
      20. The chimeric antigen receptor of any of embodiments 1-19, wherein:
the VH region comprises a CDR-H1, CDR-H2, and CDR-H3 comprising the amino acid sequences of SEQ ID NOS:80, 81 and 77, respectively, and the VL region comprises a CDR-L1, CDR-L2, and CDR-L3 comprising the amino acid sequences of SEQ ID NOS:85, 86 and 87, respectively;
the VH region comprises a CDR-H1, CDR-H2, and CDR-H3 comprising the amino acid sequences of SEQ ID NOS:82, 83 and 84, respectively, and the VL region comprises a CDR-L1, CDR-L2, and CDR-L3 comprising the amino acid sequences of SEQ ID NOS:88, 89 and 87, respectively;
the VH region comprises a CDR-H1, CDR-H2, and CDR-H3 comprising the amino acid sequences of SEQ ID NOS:95, 96, 92, respectively, and the VL region comprises a CDR-L1, CDR-L2, and CDR-L3 comprising the amino acid sequences of SEQ ID NOS:100, 101 and 102, respectively;
the VH region comprises a CDR-H1, CDR-H2, and CDR-H3 comprising the amino acid sequences of SEQ ID NOS:97, 98 and 99, respectively, and the VL region comprises a CDR-L1, CDR-L2, and CDR-L3 comprising the amino acid sequences of SEQ ID NOS:103, 104 and 102, respectively;
the VH region comprises a CDR-H1, CDR-H2, and CDR-H3 comprising the amino acid sequences of SEQ ID NOS:125, 126 and 122, respectively, and the VL region comprises a CDR-L1, CDR-L2, and CDR-L3 comprising the amino acid sequences of SEQ ID NOS:130, 131 and 132, respectively;
the VH region comprises a CDR-H1, CDR-H2, and CDR-H3 comprising the amino acid sequences of SEQ ID NOS:127, 128 and 129, respectively, and the VL region comprises a CDR-L1, CDR-L2, and CDR-L3 comprising the amino acid sequences of SEQ ID NOS:133, 134 and 132, respectively;
the VH region comprises a CDR-H1, CDR-H2, and CDR-H3 comprising the amino acid sequences of SEQ ID NOS:140, 154 and 151, respectively, and the VL region comprises a CDR-L1, CDR-L2, and CDR-L3 comprising the amino acid sequences of SEQ ID NOS:157, 158 and 159, respectively; or
the VH region comprises a CDR-H1, CDR-H2, and CDR-H3 comprising the amino acid sequences of SEQ ID NOS:142, 155 and 156, respectively, and the VL region comprises a CDR-L1, CDR-L2, and CDR-L3 comprising the amino acid sequences of SEQ ID NOS:160, 161 and 159, respectively.
   21. The chimeric antigen receptor of any of embodiments 1-20, wherein:
the VH region and the VL region comprise the amino acid sequence set forth in SEQ ID NOs:21 and 22, respectively or the amino acid sequence set forth in SEQ ID Nos: 21 and 63, respectively;
the VH region and the VL region comprise the amino acid sequence set forth in SEQ ID NOs:23 and 24, respectively or the amino acid sequence set forth in SEQ ID Nos: 23 and 64, respectively;
the VH region and the VL region comprise the amino acid sequence set forth in SEQ ID NOs:27 and 28, respectively or the amino acid sequence set forth in SEQ ID Nos: 27 and 66, respectively; or
the VH region and the VL region comprise the amino acid sequence set forth in SEQ ID NOs:31 and 32, respectively or the amino acid sequence set forth in SEQ ID Nos: 31 and 68, respectively.
   22. The chimeric antigen receptor of any of embodiments 1-21, wherein the extracellular antigen-binding domain is cross-reactive or binds mouse GPRC5D.
   23. The chimeric antigen receptor of any of embodiments 1-22, wherein the extracellular antigen-binding domain is cross-reactive or binds cynomolgus GPRC5D.
   24. The chimeric antigen receptor of any of embodiments 1-21, wherein the extracellular antigen-binding domain is not cross-reactive to or does not bind mouse GPRC5D or cynomolgus GPRC5D.
   25. The chimeric antigen receptor of any of embodiments 1-21 and 24, wherein:
the VH region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the VL region comprises the amino acid sequence set forth in SEQ ID NO:28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:28; or
the VH region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the VL region comprises the amino acid sequence set forth in SEQ ID NO:66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:66.
   26. The chimeric antigen receptor of any of embodiments 1-21, 24 and 25, comprising a variable heavy chain (VH) region comprising a CDR-H1, CDR-H2 and CDR-H3 contained within the VH region amino acid sequence set forth in SEQ ID NO: 27; and a variable light chain (VL) region comprising a CDR-L1, CDR-L2 and CDR-L3 contained within the VL region amino acid sequence set forth in SEQ ID NO: 28 or 66;
   27. The chimeric antigen receptor of any of embodiments 1-21 and 24-26, wherein the VH region comprises the amino acid sequence of SEQ ID NOS:125, 126 and 122, respectively, and the VL region comprises the amino acid sequence of SEQ ID NOS:130, 131 and 132, respectively.
   28. The chimeric antigen receptor of any of embodiments 1-21 and 24-26, wherein the VH region comprises the amino acid sequence of SEQ ID NOS:127, 128 and 129, respectively, and the VL region comprises the amino acid sequence of SEQ ID NOS:133, 134 and 132, respectively.
   29. The chimeric antigen receptor of any of embodiments 1-21 and 24-26, wherein the VH region comprises the amino acid sequence of SEQ ID NOS:120, 121 and 122, respectively, and the VL region comprises the amino acid sequence of SEQ ID NOS:130, 131 and 132, respectively.
   30. The chimeric antigen receptor of any of embodiments 1-21 and 24-26, wherein the VH region comprises the amino acid sequence of SEQ ID NOS:123, 124 and 122, respectively, and the VL region comprises the amino acid sequence of SEQ ID NOS:130, 131 and 132, respectively.
   31. The chimeric antigen receptor of any of embodiments 1-21 and 24-30, wherein the VH region and the VL regions comprise the amino acid sequence set forth in SEQ ID NOs:27 and 28, respectively or the amino acid sequence set forth in SEQ ID Nos: 27 and 66, respectively.
   32. The chimeric antigen receptor of any of embodiments 1-31, wherein the extracellular antigen-binding domain is a single chain antibody fragment.
   33. The chimeric antigen receptor of any of embodiments 1-32, wherein the single chain antibody fragment is or comprises a single chain variable fragment (scFv).
   34. The chimeric antigen receptor of any of embodiments 1-33, when the VH region and the VL region are joined by a flexible linker.
   35. The chimeric antigen receptor of embodiment 34, wherein the linker comprises the amino acid sequence GGGGSGGGGSGGGGS (SEQ ID NO:52).
   36. The chimeric antigen receptor of any of embodiments 1-35, wherein the VH region is amino-terminal to the VL region.
   37. The chimeric antigen receptor of any of embodiments 1-36, wherein:
the extracellular antigen-binding domain comprises an amino acid sequence selected from SEQ ID NOs: 1, 3, 5, 7, 9, 11, and 13 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 1, 3, 5, 7, 9, 11, and 13; and/or
the extracellular antigen-binding domain is encoded by the nucleotide sequence selected from SEQ ID Nos: 257, 259, 261, 263, 265, 267, and 269 or a nucleotide sequence having at least at or about 90%, at or about 91%, at or about 92%, at or about 93%, at or about 94%, at or about 95%, at or about 96%, at or about 97%, at or about 98%, or at or about 99% sequence identity to the nucleotide sequence selected from SEQ ID Nos: 257, 259, 261, 263, 265, 267, and 269.
   38. The chimeric antigen receptor of any of embodiments 1-37, wherein the extracellular antigen-binding domain comprises the amino acid sequence selected from SEQ ID NOs: 1, 3, 5, 7, 9, 11, and 13.
   39. The chimeric antigen receptor of any of embodiments 1-37, wherein the extracellular antigen-binding domain comprises the amino acid sequence selected from SEQ ID NOs: 1, 3, 7, and 11 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 1, 3, 7, and 11.
   40. The chimeric antigen receptor of any of embodiments 1-37 and 39, wherein the antigen-binding domain comprises the amino acid sequence selected from SEQ ID NOs: 1, 3, 7, and 11.
   41. The chimeric antigen receptor of any of embodiments 1-36 and 38, wherein the antigen-binding domain comprises the amino acid sequence set forth in SEQ ID NO: 7 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 7.
   42. The chimeric antigen receptor of any of embodiments 1-37, 39 and 41, wherein the antigen-binding domain comprises the amino acid sequence set forth in SEQ ID NO: 7.
   43. The chimeric antigen receptor of any of embodiments 1-35, wherein the VH region is carboxy-terminal to the VL region.
   44. The chimeric antigen receptor of any of embodiments 1-35 and 43, wherein:
the extracellular antigen-binding domain comprises the amino acid sequence selected from SEQ ID NOs: 2, 4, 6, 8, 10, 12, and 14 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 2, 4, 6, 8, 10, 12, and 14; and/or
the extracellular antigen-binding domain is encoded by the nucleotide sequence selected from SEQ ID Nos: 258, 260, 262, 264, 266, 268, and 270 or a nucleotide sequence having at least at or about 90%, at or about 91%, at or about 92%, at or about 93%, at or about 94%, at or about 95%, at or about 96%, at or about 97%, at or about 98%, or at or about 99% sequence identity to the nucleotide sequence selected from SEQ ID Nos: 258, 260, 262, 264, 266, 268, and 270.
   45. The chimeric antigen receptor of any of embodiments 1-35, 43 and 44, wherein the extracellular antigen-binding domain comprises the amino acid sequence selected from SEQ ID NOs: 2, 4, 6, 8, 10, 12, and 14.
   46. The chimeric antigen receptor of any of embodiments 1-35, 43 and 44, wherein the extracellular antigen-binding domain comprises the amino acid sequence selected from SEQ ID NOs: 2, 4, 8, and 12 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 2, 4, 8, and 12.
   47. The chimeric antigen receptor of any of embodiments 1-35, 43, 44 and 46, wherein the extracellular antigen-binding domain comprises the amino acid sequence selected from SEQ ID NOs: 2, 4, 8, and 12.
   48. The chimeric antigen receptor of any of embodiments 1-35, 43, 44 and 46, wherein the antigen-binding domain comprises the amino acid sequence set forth in SEQ ID NO: 8 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 8.
   49. The chimeric antigen receptor of any of embodiments 1-35, 43, 44, 46 and 48, wherein the extracellular antigen-binding domain comprises the amino acid sequence set forth in SEQ ID NO: 8.
   50. The chimeric antigen receptor of any of embodiments 1-49, wherein the intracellular signaling region comprises an intracellular cytoplasmic signaling domain.
   51. The chimeric antigen receptor of embodiment 50, wherein the intracellular signaling domain is capable of inducing a primary activation signal in a T cell, is a T cell receptor (TCR) component and/or comprises an immunoreceptor tyrosine-based activation motif (ITAM).
   52. The chimeric antigen receptor of embodiment 50 or embodiment 51, wherein the intracellular signaling domain is or comprises a cytoplasmic signaling domain of a CD3-zeta (CD3ζ) chain or a functional variant or signaling portion thereof.
   53. The chimeric antigen receptor of any of embodiments 50-52, wherein the intracellular signaling domain is human or is from a human protein.
   54. The chimeric antigen receptor of any of embodiments 50-53, wherein the intracellular signaling domain is or comprises the amino acid sequence set forth in SEQ ID NO:20 or an amino acid that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.
   55. The chimeric antigen receptor of any of embodiments 50-54, wherein the intracellular signaling region further comprises a costimulatory signaling region.
   56. The chimeric antigen receptor of embodiment 55, wherein the costimulatory signaling region comprises an intracellular signaling domain of a T cell costimulatory molecule or a signaling portion thereof.
   57. The chimeric antigen receptor of embodiment 55 or embodiment 56, wherein the costimulatory signaling region comprises an intracellular signaling domain of CD28, 4-1BB, or ICOS, or a signaling portion thereof.
   58. The chimeric antigen receptor of any of embodiments 55-57, wherein the costimulatory signaling region is human or is from a human protein.
   59. The chimeric antigen receptor of any of embodiments 55-58, wherein the costimulatory signaling region comprises an intracellular signaling domain of CD28.
   60. The chimeric antigen receptor of any of embodiments 55-59, wherein the costimulatory signaling region is or comprises the amino acid sequence set forth in SEQ ID NO:46 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.
   61. The chimeric antigen receptor of any of embodiments 55-58, wherein the costimulatory signaling region comprises an intracellular signaling domain of 4-1BB.
   62. The chimeric antigen receptor of any of embodiments 55-58 and 61, wherein the costimulatory signaling region is or comprises the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.
   63. The chimeric antigen receptor of any of embodiments 55-62, wherein the costimulatory signaling region is between the transmembrane domain and the intracellular signaling region.
   64. The chimeric antigen receptor of any of embodiments 1-63, wherein the transmembrane domain is or comprises a transmembrane domain from CD4, CD28, or CD8.
   65. The chimeric antigen receptor of any of embodiments 1-64, wherein the transmembrane domain is or comprises a transmembrane domain derived from CD28.
   66. The chimeric antigen receptor of any of embodiments 1-65, wherein the transmembrane domain is human or is from a human protein.
   67. The chimeric antigen receptor of any of embodiments 1-66, wherein the transmembrane domain is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:18.
   68. A chimeric antigen receptor comprising:
(1) an extracellular antigen-binding domain that specifically binds human G-protein coupled receptor class C group 5 member D (GPRC5D), wherein the extracellular antigen-binding domain comprises:
   (i) a variable heavy chain (VH) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 27; and
   (ii) a variable light chain (VL) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 28 or 66;
(2) a spacer comprising an IgG4/2 chimeric hinge or a modified IgG4 hinge; an IgG2/4 chimeric CH2 region; and an IgG4 CH3 region, optionally that is about 228 amino acids in length or a spacer set forth in SEQ ID NO:17;
(3) a transmembrane domain from human CD28; and
(4) an intracellular signaling region comprising a cytoplasmic signaling domain of a CD3-zeta (CD3ζ) chain and an intracellular signaling domain of a T cell costimulatory molecule.
   69. The chimeric antigen receptor of embodiment 68, wherein:
the VH region comprises a CDR-H1, CDR-H2 and CDR-H3 contained within the VH region amino acid sequence set forth in SEQ ID NO: 27; and the VL region comprises a CDR-L1, CDR-L2 and CDR-L3 contained within the VL region amino acid sequence set forth in SEQ ID NO: 28 or 66; or
the VH region a CDR-H1, CDR-H2, and CDR-H3 comprising the amino acid sequence of SEQ ID NOS:125, 126 and 122, respectively, and the VL region comprises a CDR-L1, CDR-L2, and CDR-L3 comprising the amino acid sequence of SEQ ID NOS:130, 131 and 132, respectively;
the VH region comprises a CDR-H1, CDR-H2, and CDR-H3 comprising the amino acid sequence of SEQ ID NOS:127, 128 and 129, respectively, and the VL region comprises a CDR-L1, CDR-L2, and CDR-L3 comprising the amino acid sequence of SEQ ID NOS:133, 134 and 132, respectively;
the VH region comprises a CDR-H1, CDR-H2, and CDR-H3 comprising the amino acid sequence of SEQ ID NOS:120, 121 and 122, respectively, and the VL region comprises a CDR-L1, CDR-L2, and CDR-L3 comprising the amino acid sequence of SEQ ID NOS:130, 131 and 132, respectively; or
the VH region comprises a CDR-H1, CDR-H2, and CDR-H3 comprising the amino acid sequence of SEQ ID NOS:123, 124 and 122, respectively, and the VL region comprises a CDR-L1, CDR-L2, and CDR-L3 comprising the amino acid sequence of SEQ ID NOS:130, 131 and 132, respectively.
   70. A chimeric antigen receptor comprising:
(1) an extracellular antigen-binding domain that specifically binds human G-protein coupled receptor class C group 5 member D (GPRC5D), wherein the extracellular antigen-binding domain comprises:
a VH region comprising a CDR-H1, CDR-H2 and CDR-H3 contained within the VH region amino acid sequence set forth in SEQ ID NO: 27; and a variable light (VL) region comprising a CDR-L1, CDR-L2 and CDR-L3 contained within the VL region amino acid sequence set forth in SEQ ID NO: 28 or 66; or
a VH region comprising a CDR-H1, CDR-H2, and CDR-H3 comprising the amino acid sequence of SEQ ID NOS:125, 126 and 122, respectively, and a VL region comprising a CDR-L1, CDR-L2, and CDR-L3 comprising the amino acid sequence of SEQ ID NOS:130, 131 and 132, respectively;
a VH region comprising a CDR-H1, CDR-H2, and CDR-H3 comprising the amino acid sequence of SEQ ID NOS:127, 128 and 129, respectively, and a VL region comprising a CDR-L1, CDR-L2, and CDR-L3 comprising the amino acid sequence of SEQ ID NOS:133, 134 and 132, respectively;
a VH region comprising a CDR-H1, CDR-H2, and CDR-H3 comprising the amino acid sequence of SEQ ID NOS:120, 121 and 122, respectively, and a VL region comprising a CDR-L1, CDR-L2, and CDR-L3 comprising the amino acid sequence of SEQ ID NOS:130, 131 and 132, respectively; or
a VH region comprising a CDR-H1, CDR-H2, and CDR-H3 comprising the amino acid sequence of SEQ ID NOS:123, 124 and 122, respectively, and a VL region comprising a CDR-L1, CDR-L2, and CDR-L3 comprising the amino acid sequence of SEQ ID NOS:130, 131 and 132, respectively;
(2) a spacer comprising an IgG4/2 chimeric hinge or a modified IgG4 hinge; an IgG2/4 chimeric CH2 region; and an IgG4 CH3 region, optionally that is about 228 amino acids in length or a spacer set forth in SEQ ID NO:17;
(3) a transmembrane domain from human CD28; and
(4) an intracellular signaling region comprising a cytoplasmic signaling domain of a human CD3-zeta (CD3ζ) chain and an intracellular signaling domain of a human CD28 or a human 4-1BB.
   71. The chimeric antigen receptor of any of embodiments 68-70, wherein:
the extracellular antigen-binding domain comprises the VH region amino acid sequence set forth in SEQ ID NO:27 and the VL region amino acid sequence set forth in SEQ ID NO:28 or 66; and/or
the extracellular antigen-binding domain comprises an scFv set forth in SEQ ID NO:7 or SEQ ID NO:8.
   72. The chimeric antigen receptor of any of embodiments 68-71, wherein the transmembrane domain is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.
   73. The chimeric antigen receptor of embodiment 72, wherein the transmembrane domain is or comprises the sequence set forth in SEQ ID NO:18.
   74. The chimeric antigen receptor of any of embodiments 68-73, wherein the intracellular signaling region comprises (a) the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20 and (b) the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.
   75. The chimeric antigen receptor of any of embodiments 68-74, wherein the intracellular signaling region is or comprises the sequences set forth in SEQ ID NO:20 and SEQ ID NO:46.
   76. The chimeric antigen receptor of any of embodiments 68-73, wherein the intracellular signaling region comprises (a) the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20 and (b) the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.
   77. The chimeric antigen receptor of any of embodiments 68-73 and 76, wherein the intracellular signaling region is or comprises the sequences set forth in SEQ ID NO:20 and SEQ ID NO:19.
   78. The chimeric antigen receptor of any of embodiments 1-77, wherein the encoded chimeric antigen receptor comprises from its N to C terminus in order: the antigen-binding domain, the spacer, the transmembrane domain and the intracellular signaling region.
   79. A polynucleotide comprising a nucleotide sequence encoding the chimeric antigen receptor of any of embodiments 1-78.
   80. The polynucleotide of embodiment 79, wherein the nucleic acid encoding the spacer comprises at least one modified splice donor and/or splice acceptor site, said modified splice donor and/or acceptor site comprising one or more nucleotide modifications corresponding to a reference splice donor site and/or reference splice acceptor site contained in the sequence set forth in SEQ ID NO:73.
   81. The polynucleotide of embodiment 80, wherein the one or more nucleotide modifications comprise an amino acid substitution.
   82. The polynucleotide of embodiment 80 or embodiment 81, wherein the reference splice donor and/or reference splice acceptor sites are canonical, non-canonical, or cryptic splice sites.
   83. The polynucleotide of any of embodiment 80-82, wherein:
the reference splice donor and/or reference splice acceptor site(s) has a splice site prediction score of at least at or about 0.4, at or about 0.5, at or about 0.6, at or about 0.70, at or about 0.75, at or about 0.80, at or about 0.85, at or about 0.90, at or about 0.95, at or about 0.99, or at or about 1.0; and/or
the reference splice donor and/or reference splice acceptor site(s) is/are predicted to be involved in a splice event with a probability of at least at or about 40%, at or about 50%, at or about 60%, at or about 70%, at or about 75%, at or about 80%, at or about 85%, at or about 90%, at or about 95%, at or about 99%, or at or about 100%.
   84. The polynucleotide of any of embodiments 80-83, wherein:
the reference splice donor site comprises the sequence aatctaagtacggac (SEQ ID NO: 176), tcaactggtacgtgg (SEQ ID NO:177), acaattagtaaggca (SEQ ID NO:178) and/or accacaggtgtatac (SEQ ID NO:179); and/or
the reference splice acceptor site comprises the sequence aagtttctttctgtattccaggctgaccgtggataaatctc (SEQ ID NO:180) and/or gggcaacgtgttctcttgcagtgtcatgcacgaagccctgc (SEQ ID NO:181).
   85. The polynucleotide of any of embodiment 80-84, wherein:
the reference splice donor and/or reference splice acceptor site(s) has a splice site prediction score of at least at or about 0.70, at or about 0.75, at or about 0.80, at or about 0.85, at or about 0.90, 0.95, at or about 0.99, or at or about 1.0; and/or
the reference splice donor and/or reference splice acceptor site(s) is/are predicted to be involved in a splice event with a probability of at least at or about 70%, at or about 75%, at or about 80%, at or about 85%, at or about 90%, at or about 95%, at or about 99%, or at or about 100%.
   86. The polynucleotide of any of embodiments 80-85, wherein:
the reference splice donor site comprises the sequence tcaactggtacgtgg (SEQ ID NO:177); and/or
the reference splice acceptor site comprises the sequence aagtttctttctgtattccaggctgaccgtggataaatctc (SEQ ID NO:180).
   87. The polynucleotide of any of embodiments 80-86, wherein at least one of the one or more nucleotide modifications are within 1, 2, 3, 4, 5, 6, 7, 8, 9 or 10 residues of the splice site junction of the reference splice acceptor and/or reference splice donor site.
   88. The polynucleotide of any of embodiments 80-87, wherein the one or more nucleotide modifications is silent and/or results in a degenerate codon compared to SEQ ID NO:73 and/or does not change the amino acid sequence of the encoded spacer.
   89. The polynucleotide of any of embodiments 80-88, wherein:
the modified splice donor site is set forth in agtctaaatacggac (SEQ ID NO:182), tcaactggtatgtgg (SEQ ID NO:183), accatctccaaggcc (SEQ ID NO:184) and/or gccccaggtttacac (SEQ ID NO:185); and/or
the modified splice acceptor site is set forth in cagtttcttcctgtatagtagactcaccgtggataaatcaa (SEQ ID NO:186), gggcaacgtgttcagctgcagcgtgatgcacgaggccctgc (SEQ ID NO: 187) and/or cgccttgtcctccttgtcccgctcctcctgttgccggacct (SEQ ID NO:188).
   90. The polynucleotide of any of embodiments 80-89, wherein the modified splice donor site is set forth in tcaactggtatgtgg (SEQ ID NO:183) and/or the modified acceptor site is set forth in cagtttcttcctgtatagtagactcaccgtggataaatcaa (SEQ ID NO:186) and/or cgccttgtcctccttgtcccgctcctcctgttgccggacct (SEQ ID NO:188).
   91. The polynucleotide of any of embodiments 80-90, wherein the spacer is encoded by the nucleotide sequence set forth in SEQ ID NO:74 or a portion thereof.
   92. The polynucleotide of any of embodiments 79-91, wherein upon expression of the polynucleotide in a cell, the transcribed RNA, optionally messenger RNA (mRNA), from the polynucleotide, exhibits at least at or about 70%, at or about 75%, at or about 80%, at or about 85%, at or about 90%, or at or about 95% RNA homogeneity.
   93. The polynucleotide of any of embodiments 79-92, wherein, upon expression in a cell, the transcribed RNA, optionally messenger RNA (mRNA), from the polynucleotide exhibits reduced heterogeneity compared to the heterogeneity of the mRNA transcribed from a reference polynucleotide, said reference polynucleotide encoding the same amino acid sequence as the polynucleotide, wherein the reference polynucleotide differs by the presence of one or more splice donor site and/or one or more splice acceptor site in the nucleic acid encoding the spacer and/or comprises one or more nucleotide modifications compared to the polynucleotide and/or comprises the spacer set forth in SEQ ID NO:73.
   94. The polynucleotide of embodiment 93, wherein the RNA heterogeneity is reduced by greater than at or about 10%, at or about 15%, at or about 20%, at or about 25%, at or about 30%, at or about 40%, at or about 50% or more.
   95. The polynucleotide of embodiment 93 or embodiment 94, wherein the transcribed RNA, optionally messenger RNA (mRNA), from the reference polynucleotide exhibits greater than at or about 10%, at or about 15%, at or about 20%, at or about 25%, at or about 30%, at or about 40%, at or about 50% or more RNA heterogeneity.
   96. The polynucleotide of any of embodiments 79-95, wherein the RNA homogeneity and/or heterogeneity is determined by agarose gel electrophoresis, chip-based capillary electrophoresis, analytical ultracentrifugation, field flow fractionation, or liquid chromatography.
   97. The polynucleotide of any of embodiments 79-96, wherein the polynucleotide is codon-optimized for expression in a human cell.
   98. The polynucleotide of any of embodiments 79-96, wherein the chimeric antigen receptor is a first chimeric antigen receptor and the polynucleotide further comprises a nucleotide sequence encoding a second chimeric antigen receptor.
   99. The polynucleotide of embodiment 98, wherein the first and second chimeric antigen receptors are separated by one or more multicistronic element(s).
   100. The polynucleotide of embodiment 99, wherein the one or more multicistronic element is or comprises a ribosome skip sequence, optionally wherein the ribosome skip sequence is a T2A, a P2A, an E2A, or an F2A element.
   101. The polynucleotide of embodiment 100, wherein the nucleotide sequence encoding the one or more multicistronic element is codon diverged.
   102. The polynucleotide of embodiment 100 or embodiment 101, wherein the nucleotide sequence encoding the one or more multicistronic element is or comprises the sequence set forth in SEQ ID NO:319. 103. The polynucleotide of any of embodiments 98-102, wherein the second chimeric antigen receptor (CAR) comprises an extracellular antigen-binding domain that specifically binds a second antigen expressed on or associated with multiple myeloma.
   104. The polynucleotide of 103, wherein the second CAR further comprises a spacer, a transmembrane domain, and an intracellular signaling region.
   105. The polynucleotide of embodiment 103 or embodiment 104, wherein the second antigen is selected from B cell maturation antigen (BCMA), CD38, CD138, CS-1, BAFF-R, TACI, and FcRH5.
   106. The polynucleotide of any of embodiments 103-105, wherein the second antigen is BCMA.
   107. The polynucleotide of any of embodiments 103-106, wherein the second CAR comprises:
(1) an extracellular antigen-binding domain that specifically binds BCMA, wherein the extracellular antigen-binding domain comprises:
   (i) a variable heavy chain (VH) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 189, 191, 193, 195 or 197; and
   (ii) a variable light chain (VL) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 190, 192, 194, 196 or 198;
(2) a spacer, optionally a spacer comprising an IgG4/2 chimeric hinge or a modified IgG4 hinge; an IgG2/4 chimeric CH2 region; and an IgG4 CH3 region, optionally that is about 228 amino acids in length, or a spacer set forth in SEQ ID NO:17;
(3) a transmembrane domain; and
(4) an intracellular signaling region.
   108. The polynucleotide of embodiment 107, wherein the VH region of the second CAR comprises a CDR-H1, CDR-H2 and CDR-H3 contained within the VH region amino acid sequence set forth in SEQ ID NO: 189, 191, 193, 195 or 197; and the VL region of the second CAR comprises a CDR-L1, CDR-L2 and CDR-L3 contained within the VL region amino acid sequence set forth in SEQ ID NO: 190, 192, 194, 196 or 198.
   109. The polynucleotide of any of embodiments 103-106, wherein the second CAR comprises:
(1) an extracellular antigen-binding domain that specifically binds BCMA, wherein the extracellular antigen-binding domain comprises:
   (i) a variable heavy chain (VH) region comprising a heavy chain complementarity determining region 1 (CDR-H1) comprising the amino acid sequence selected from SEQ ID NOs: 199, 202, 206, 209, 212, and 215; (b) a heavy chain complementarity determining region 2 (CDR-H2) comprising the amino acid sequence selected from SEQ ID NOs: 200, 203, 207, 210, 213, and 216; and (c) a heavy chain complementarity determining region 3 (CDR-H3) comprising the amino acid sequence selected from SEQ ID NOs: 201, 204, 205, 208, 211, 214, and 217; and
   (ii) a variable light chain (VL) region comprising a light chain complementarity determining region 1 (CDR-L1) comprising the amino acid sequence selected from SEQ ID NOs: 218, 221, 224, 227, 230, 233, and 235; (b) a light chain complementarity determining region 2 (CDR-L2) comprising the amino acid sequence selected from any one of SEQ ID NOs: 219, 222, 225, 228, 231, 234, and 236; and (c) a light chain complementarity determining region 3 (CDR-L3) comprising the amino acid sequence selected from SEQ ID NOs: 220, 223, 226, 229, and 232;
(2) a spacer, optionally a spacer comprising an IgG4/2 chimeric hinge or a modified IgG4 hinge; an IgG2/4 chimeric CH2 region; and an IgG4 CH3 region, optionally that is at or about 228 amino acids in length, or a spacer set forth in SEQ ID NO:17;
(3) a transmembrane domain; and
(4) an intracellular signaling region.
   110. The polynucleotide of any of embodiments 107-109, wherein:
the VH region of the second CAR comprises a CDR-H1, CDR-H2, and CDR-H3 comprising the amino acid sequence of SEQ ID NOS:199, 200 and 201, respectively, and the VL region of the second CAR comprises a CDR-L1, CDR-L2, and CDR-L3 comprising the amino acid sequence of SEQ ID NOS:218, 219 and 220, respectively;
the VH region of the second CAR comprises a CDR-H1, CDR-H2, and CDR-H3 comprising the amino acid sequence of SEQ ID NOS:202, 203, 204, respectively, and the VL region of the second CAR comprises a CDR-L1, CDR-L2, and CDR-L3 comprising the amino acid sequence of SEQ ID NOS:221, 222 and 223, respectively;
the VH region of the second CAR comprises a CDR-H1, CDR-H2, and CDR-H3 comprising the amino acid sequence of SEQ ID NOS:199, 200, 205, respectively, and the VL region of the second CAR comprises a CDR-L1, CDR-L2, and CDR-L3 comprising the amino acid sequence of SEQ ID NOS:224, 225, and 226, respectively;
the VH region of the second CAR comprises a CDR-H1, CDR-H2, and CDR-H3 comprising the amino acid sequence of SEQ ID NOS:206, 207, 208, respectively, and the VL region of the second CAR comprises a CDR-L1, CDR-L2, and CDR-L3 comprising the amino acid sequence of SEQ ID NOS:227, 228 and 229, respectively;
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively;
the VH region of the second CAR comprises a CDR-H1, CDR-H2, and CDR-H3 comprising the amino acid sequence of SEQ ID NOS:212, 213 and 214, respectively, and the VL region of the second CAR comprises a CDR-L1, CDR-L2, and CDR-L3 comprising the amino acid sequence of SEQ ID NOS:233, 234 and 229, respectively; or
the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively.
   111. The polynucleotide of any of embodiments 107-109, wherein:
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively; or
the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively.
   112. The polynucleotide of any of embodiments 107-111, wherein:
the VH region and VL region of the second CAR comprise (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the VH region and VL region of the second CAR comprise (a) the amino acid sequences set forth in SEQ ID NO:191 and SEQ ID NO:192, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the VH region and VL region of the second CAR comprise (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the VH region and VL region of the second CAR comprise (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the VH region and VL region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:198.
   113. The polynucleotide of any of embodiments 107-112, wherein:
the VH region and VL region of the second CAR comprise the amino acid sequences set forth in SEQ ID NO:189 and SEQ ID NO:190, respectively;
the VH region and VL region of the second CAR comprise the amino acid sequences set forth in SEQ ID NO:191 and SEQ ID NO:192;
the VH region and VL region of the second CAR comprise the amino acid sequences set forth in SEQ ID NO:193 and SEQ ID NO:194;
the VH region and VL region of the second CAR comprise the amino acid sequences set forth in SEQ ID NO:195 and SEQ ID NO:196; or
the VH region and VL region of the second CAR comprise the amino acid sequences set forth in SEQ ID NO:197 and SEQ ID NO:198, respectively.
   114. The polynucleotide of any of embodiments 107-113, wherein the extracellular antigen-binding domain of the second CAR is a single chain antibody fragment.
   115. The polynucleotide of embodiment 114, wherein the single chain antibody fragment is or comprises a single chain variable fragment (scFv).
   116. The polynucleotide of any of embodiments 107-115, when the VH region and the VL region of the second CAR are joined by a flexible linker.
   117. The polynucleotide of embodiment 116, wherein the linker of the second CAR comprises the amino acid sequence GGGGSGGGGSGGGGS (SEQ ID NO:52).
   118. The polynucleotide of any of embodiments 107-117, wherein the VH region is amino-terminal to the VL region in the second CAR.
   119. The polynucleotide of any of embodiments 107-117, wherein the VH region is carboxy-terminal to the VL region in the second CAR.
   120. The polynucleotide of any of embodiments 107-119, wherein the antigen-binding domain of the second CAR comprises the amino acid sequence selected from SEQ ID NOs: 237, 238, 239, 240, and 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 237, 238, 239, 240, and 241.
   121. The polynucleotide of any of embodiments 107-120, wherein the antigen-binding domain of the second CAR comprises the amino acid sequence selected from SEQ ID NOs: 237, 238, 239, 240, and 241.
   122. The polynucleotide of any of embodiments 107-121, wherein:
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively; or the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively; and/or
the VH region and VL region of the second CAR comprises the amino acid sequences set forth in SEQ ID NO:197 and SEQ ID NO:198, respectively; and/or
the extracellular antigen-binding domain of the second CAR comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.
   123. The polynucleotide of any of embodiments 104-122, wherein the transmembrane domain of the second CAR is or comprises a transmembrane domain from CD4, CD28, or CD8, optionally from human CD4, human CD28 or human CD8.
   124. The polynucleotide of any of embodiments 104-123, wherein:
the transmembrane domain of the second CAR is or comprises a transmembrane domain from human CD28; and/or
the transmembrane domain of the second CAR is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.
   125. The polynucleotide of embodiment 124, wherein the transmembrane domain of the second CAR is or comprises the sequence set forth in SEQ ID NO:18.
   126. The polynucleotide of any of embodiments 104-125, wherein the intracellular signaling region of the second CAR comprises an intracellular signaling domain.
   127. The polynucleotide of embodiment 126, wherein the intracellular signaling domain of the second CAR is capable of inducing a primary activation signal in a T cell, is a T cell receptor (TCR) component and/or comprises an immunoreceptor tyrosine-based activation motif (ITAM).
   128. The polynucleotide of embodiment 126 or embodiment 127, wherein the intracellular signaling domain of the second CAR is or comprises a cytoplasmic signaling domain of a CD3-zeta (CD3ζ) chain or a functional variant or signaling portion thereof, optionally a human CD3 zeta chain.
   129. The polynucleotide of any of embodiments 126-128, wherein the intracellular signaling region of the second CAR comprises the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.
   130. The polynucleotide of any of embodiments 126-129, wherein the intracellular signaling region of the second CAR further comprises a costimulatory signaling region.
   131. The polynucleotide of embodiment 130, wherein the costimulatory signaling region of the second CAR comprises an intracellular signaling domain of a T cell costimulatory molecule or a signaling portion thereof.
   132. The polynucleotide of embodiment 130 or embodiment 131, wherein the costimulatory signaling region of the second CAR comprises an intracellular signaling domain of CD28, 4-1BB, or ICOS, or a signaling portion thereof, optionally of human CD28, human 4-1BB, or human ICOS.
   133. The polynucleotide of any of embodiments 98-132, wherein at least one of the first chimeric antigen receptor and the second chimeric antigen receptor comprises an intracellular signaling region comprising an intracellular signaling domain of 4-1BB or a signaling portion thereof, optionally of human 4-1BB.134. The polynucleotide of any of embodiments 130-132, wherein the costimulatory signaling region of the second CAR comprises:
an intracellular signaling domain of human CD28; and/or
the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.
   135. The polynucleotide of any of embodiments 130-133, wherein the costimulatory signaling region comprises:
an intracellular signaling domain of human 4-1BB; and/or
the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.
   136. The polynucleotide of any of embodiments 104-135, wherein the second chimeric antigen receptor comprises from its N to C terminus in order: the extracellular antigen-binding domain, the spacer, the transmembrane domain and the intracellular signaling region.
   137. The polynucleotide of any of embodiments 79-97, wherein the sequence of nucleotides encoding the CAR is operably linked to a promoter to control expression of the encoded CAR when expressed from a cell introduced with the polynucleotide, optionally wherein the promoter is a heterologous promoter, optionally wherein the heterologous promoter is or comprises a human elongation factor 1 alpha (EF1α) promoter or an MND promoter or a variant thereof.
   138. The polynucleotide of any of embodiments 98-136, wherein the nucleotide sequence encoding the first CAR is operably linked to a first promoter to control expression of the first CAR when expressed from a cell introduced with the polynucleotide and the nucleotide sequence encoding the second CAR is operably linked to a second promoter to control expression of the second CAR when expressed from a cell introduced with the polynucleotide, optionally wherein the first and second promoter independently is a heterologous promoter, optionally wherein the heterologous promoter is or comprises a human elongation factor 1 alpha (EF1α) promoter or an MND promoter or a variant thereof.
   139. The polynucleotide of embodiment 138, wherein the first and second promoters are the same.
   140. The polynucleotide of embodiment 138, wherein the first and second promoters are different.
   141. A polynucleotide comprising:
(i) a first nucleic acid sequence encoding a first chimeric antigen receptor (CAR) comprising a first antigen binding domain; and
(ii) a second nucleic acid sequence encoding a second chimeric antigen receptor (CAR) comprising a second antigen binding domain;
wherein the first CAR and second CAR each comprise the following: (a) the first antigen binding domain or the second antigen binding domain, (b) a spacer, (c) a transmembrane domain, and (d) an intracellular signaling region comprising an intracellular signaling domain and a costimulatory signaling region;
   wherein one or more of (b) through (d) in the first CAR and the same one or more of (b) through (d) in the second CAR comprises the identical amino acid sequence; and
wherein the nucleotide sequence(s) encoding the one or more of (b) through (d) in the first CAR differs in sequence from the nucleotide sequence(s) encoding the same one or more of (b) through (d) in the second CAR.
   142. The polynucleotide of embodiment 141, wherein the first and second antigen binding domains bind to the same antigen.
   143. The polynucleotide of embodiment 141 or embodiment 142, wherein the first and second antigen binding domains bind different epitopes of the same antigen.
   144. The polynucleotide of embodiment 141, wherein the first and second antigen binding domains bind to different antigens.
   145. The polynucleotide of any of embodiments 141-144, wherein the first antigen binding domain binds a first antigen expressed by or associated with cells of a disease or condition and the second antigen binding domains binds a second antigen expressed by or associated with cells of the same disease or condition.
   146. The polynucleotide of embodiment 145, wherein the disease or condition is a cancer.
   147. The polynucleotide of embodiment 146, wherein the disease or condition is a GPRC5D-expressing cancer or a BCMA-expressing cancer.
   148. The polynucleotide of embodiment 146 or embodiment 147, wherein the cancer is a plasma cell malignancy and the plasma cell malignancy is multiple myeloma (MM) or plasmacytoma.
   149. The polynucleotide of any of embodiments 146-148, wherein the cancer is multiple myeloma.
   150. The polynucleotide of any of embodiments 146-149, wherein the cancer is relapsed/refractory multiple myeloma.
   151. The polynucleotide of any of embodiments 141-150, wherein the first and second antigen binding domain independently bind to an antigen selected from the group consisting of GPRC5D, BCMA, CD38 CD138, CS-1, BAFF-R, TACI and FcRH5.
   152. The polynucleotide of any of embodiments 141-151, wherein the first antigen binding domain binds to B cell maturation antigen (BCMA).
   153. The polynucleotide of any of embodiments 141-151, wherein the first antigen binding domain bind to G protein-coupled receptor class C group 5 member D (GPRC5D).
   154. The polynucleotide of embodiment 152 or embodiment 153, wherein the second antigen binding domain binds to BCMA.
   155. The polynucleotide of embodiment 152 or embodiment 153, wherein the second antigen binding domain binds to GPRC5D.
   156. A polynucleotide comprising:
(i) a first nucleic acid sequence encoding a first chimeric antigen receptor (CAR) comprising a first antigen binding domain capable of binding to one of GPRC5D or BCMA and
(ii) a second nucleic acid sequence encoding a second chimeric antigen receptor (CAR) comprising a second antigen binding domain capable of binding to the other of GPRC5D or BCMA;
wherein the first CAR and second CAR each comprise the following: (a) the first antigen binding domain or the second antigen binding domain, (b) a spacer, (c) a transmembrane domain, and (d) an intracellular signaling region comprising an intracellular signaling domain and a costimulatory signaling region;
wherein one or more of (b) through (d) in the first CAR and the same one or more of (b) through (d) in the second CAR comprises the identical amino acid sequence; and
wherein the nucleotide sequence(s) encoding the one or more of (b) through (d) in the first CAR differs in sequence from the nucleotide nucleotide sequence(s) encoding the same one or more of (b) through (d) in the second CAR.
   157. The polynucleotide of embodiment any of embodiments 141-156, wherein the one or more of (b) through (d) is one of (b) through (d).
   158. The polynucleotide of any of embodiments 141-156, wherein the one or more of (b) through (d) is two of (b) through (d).
   159. The polynucleotide of any of embodiments 141-156, wherein the one or more of (b) through (d) is each of (b) through (d).
   160. The polynucleotide of any of embodiments 141-159, wherein:
the nucleotide sequence(s) encoding the one or more of (b) through (d) in the first CAR and the nucleotide sequence(s) encoding the same one or more of (b) through (d) in the second CAR comprises no more than about 20 consecutive base pairs of sequence homolog; and/or
the first nucleic acid sequence encoding the first CAR and the second nucleic acid sequence encoding the second CAR comprise no more than about 20 consecutive base pairs of sequence homology.
   161. The polynucleotide of any of embodiments 141-160, wherein:
the nucleotide sequence(s) encoding the one or more of (b) through (d) in the first CAR and the nucleotide sequence(s) encoding the same one or more of (b) through (d) in the second CAR comprises no more than between about 5 and about 15 consecutive base pairs of sequence homology; and/or
the first nucleic acid sequence encoding the first CAR and the second nucleic acid sequence encoding the second CAR comprise no more than about 5 and about 15 consecutive base pairs of sequence homology.
   162. The polynucleotide of any of embodiments 141-161, wherein:
the nucleotide sequence(s) encoding the one or more of (b) through (d) in the first CAR and the nucleotide sequence(s) encoding the same one or more of (b) through (d) in the second CAR comprises no more than about 10 consecutive base pairs of homology; and/or
the first nucleic acid sequence encoding the first CAR and the second nucleic acid sequence encoding the second CAR comprise no more than about 10 consecutive base pairs of sequence homology.
   163. The polynucleotide of any of embodiments 141-162, wherein the first nucleic acid encoding the first CAR and the second nucleic acid encoding the second CAR are separated by a nucleotide sequence encoding a multicistronic element, optionally wherein the multicistronic element is a bicistronic element.
   164. The polynucleotide of embodiment 163, wherein the multicistronic element is an IRES or is a ribosome skip sequence or self-cleaving peptide.
   165. The polynucleotide of embodiment 164, wherein the multicistronic element is a ribosome skip sequence or self-cleaving peptide and the ribosome skip sequence or self-cleaving peptide is a T2A, a P2A, an E2A, or an F2A element.
   166. The polynucleotide of any of embodiments 141-165, wherein the first nucleic acid sequence encoding the first CAR is codon optimized for expression in a human cell.
   167. The polynucleotide of any of embodiments 141-166, wherein the second nucleic acid sequence encoding the second CAR is codon optimized for expression in a human cell.
   168. The polynucleotide of any of embodiments 141-167, wherein the polynucleotide is codon optimized for expression in a human cell.
   169. The polynucleotide of any of embodiments 141-168, wherein following transcription of the polynucleotide in a human cell, optionally a human T cell, the transcribed mRNA, optionally messenger RNA, from the polynucleotide, exhibits at least about 70%, 75%, 80%, 85%, 90%, or 95% RNA homogeneity.
   170. The polynucleotide of any of embodiments 141-169, wherein following transcription of the first nucleic acid encoding the first CAR of the polynucleotide in a human cell, optionally a human T cell, the transcribed mRNA, optionally messenger RNA, from the first nucleic acid exhibits at least about 70%, 75%, 80%, 85%, 90%, or 95% RNA homogeneity.
   171. The polynucleotide of any of embodiments 141-170, wherein following transcription of the second nucleic acid encoding the second CAR of the polynucleotide in a human cell, optionally a human T cell, the transcribed mRNA, optionally messenger RNA, from the second nucleic acid exhibits at least about 70%, 75%, 80%, 85%, 90%, or 95% RNA homogeneity.
   172. The polynucleotide of any of embodiments 141-171,
wherein any potential splice donor and/or splice acceptor site present in the first nucleic acid encoding the first CAR exhibits a splice prediction score of about or at least about less than 0.70, 0.65, 0.60, 0.55, 0.50, 0.45, 0.40, 0.35, 0.30, 0.25, 0.20 and/or is predicted to be involved in a splice event with a probability of less than 70%, less than 65%, less than 60%, less than 55%, less than 50%, less than 45%, less than 40%, less than 35%, less than 30%, less than 25%, or less than 20%.
   173. The polynucleotide of any of embodiments 141-172,
wherein any potential splice donor or acceptor site in the second nucleic acid encoding the second CAR exhibits a splice prediction score of about or at least about less than 0.70, 0.65, 0.60, 0.55, 0.50, 0.45, 0.40, 0.35, 0.30, 0.25, 0.20 and/or is predicted to be involved in a splice event with a probability of less than 70%, less than 65%, less than 60%, less than 55%, less than 50%, less than 45%, less than 40%, less than 35%, less than 30%, less than 25%, or less than 20%.
   174. The polynucleotide of any of embodiments 141-173, wherein any potential splice donor or acceptor sites in the polynucleotide exhibits a splice prediction score of about or at least about less than 0.70, 0.65, 0.60, 0.55, 0.50, 0.45, 0.40, 0.35, 0.30, 0.25, 0.20 and/or is predicted to be involved in a splice event with a probability of less than 70%, less than 65%, less than 60%, less than 55%, less than 50%, less than 45%, less than 40%, less than 35%, less than 30%, less than 25%, or less than 20%.
   175. The polynucleotide of any of embodiments 141-174, wherein the first and/or second antigen binding domain of (a) is a single chain antibody fragment.
   176. The polynucleotide of any of embodiments 141-175, wherein the first and/or second antigen binding domain of (a) is or comprises a single chain variable fragment (scFv)
   177. The polynucleotide of any of embodiments 141-176, wherein the first and/or second antigen binding domain of (a) comprises a variable heavy chain (VH) region and a variable light chain (VL) region.
   178. The polynucleotide of any of embodiments 141-177, wherein one of the first antigen binding domain or the second antigen binding domain comprises a VH region that comprises a CDR-H1 as set forth in SEQ ID NO:209, a CDR-H2 as set forth in SEQ ID NO:210, and a CDR-H3 as set forth in SEQ ID NO:211 and a VL region that comprises a CDR-L1 as set forth in SEQ ID NO:230, a CDR-L2 as set forth in SEQ ID NO:231, and a CDR-L3 as set forth in SEQ ID NO:232.
   179. The polynucleotide of any of embodiments 141-178, wherein one of the first antigen binding domain or the second antigen binding domain comprise a VH region and a VL region that comprise the amino acid sequences set forth in SEQ ID NOS:197 and 198, respectively.
   180. The polynucleotide of any of embodiments 141-179, wherein one of the first antigen binding domain or the second antigen binding domain comprises the amino acid sequence set forth in SEQ ID NO:241 or a sequence of amino acids that exhibits at least at or about 90%, at least about or about 91%, at least at or about 92%, at least at or about 93%, at least at or about 94%, at least at or about 95%, at least at or about 96%, at least at or about 97%, at least at or about 98%, at least at or about 99% sequence identity to SEQ ID NO:241.
   181. The polynucleotide of any of embodiments 141-180, wherein one of the first antigen binding domain or the second antigen binding domain comprises a VH region comprises a CDR-H1 as set forth in SEQ ID NO:125, a CDR-H2 as set forth in SEQ ID NO:126, and a CDR-H3 as set forth in SEQ ID NO:127 and a VL region that comprises a CDR-L1 as set forth in SEQ ID NO:130, a CDR-L2 as set forth in SEQ ID NO:131, and a CDR-L3 as set forth in SEQ ID NO:132.
   182. The polynucleotide of any of embodiments 141-181, wherein one of the first antigen binding domain or the second antigen binding domain comprise a VH region and VL region that comprise the amino acid sequences set forth in SEQ ID NOS:27 and 28, respectively.
   183. The polynucleotide of any of embodiments 141-182, wherein one of the first antigen binding domain or the second antigen binding domain comprises the amino acid sequence set forth in SEQ ID NO:8 or a sequence of amino acids that exhibits at least at or about 90%, at least about or about 91%, at least at or about 92%, at least at or about 93%, at least at or about 94%, at least at or about 95%, at least at or about 96%, at least at or about 97%, at least at or about 98%, at least at or about 99% sequence identity to SEQ ID NO:8.
   184. The polynucleotide of any of embodiments 141-183, wherein:
one of the first antigen binding domain or the second antigen binding domain comprises a VH region that comprises a CDR-H1 as set forth in SEQ ID NO:209, a CDR-H2 as set forth in SEQ ID NO:210, and a CDR-H3 as set forth in SEQ ID NO:211 and a VL region that comprises a CDR-L1 as set forth in SEQ ID NO:230, a CDR-L2 as set forth in SEQ ID NO:231, and a CDR-L3 as set forth in SEQ ID NO:232; and
the other of the first antigen binding domain or the second antigen binding domain comprises a CDR-H1 as set forth in SEQ ID NO:125, a CDR-H2 as set forth in SEQ ID NO:126, and a CDR-H3 as set forth in SEQ ID NO:127 and a VL region that comprises a CDR-L1 as set forth in SEQ ID NO:130, a CDR-L2 as set forth in SEQ ID NO:131, and a CDR-L3 as set forth in SEQ ID NO:132.
   185. The polynucleotide of any of embodiments 141-184, wherein:
one of the first antigen binding domain or the second antigen binding domain comprise a VH region and a VL region that comprise the amino acid sequences set forth in SEQ ID NOS:197 and 198, respectively; and
the other of the first antigen binding domain or the second antigen binding domain comprises a VH region and VL region that comprise the amino acid sequences set forth in SEQ ID NOS:27 and 28, respectively.
   186. The polynucleotide of any of embodiments 141-185, wherein one of the first or second antigen binding domain comprises the amino acid sequence set forth in SEQ ID NO:241 and the other of the first or second antigen binding domain comprises the amino acid sequence set forth in SEQ ID NO:8.
   187. The polynucleotide of any of embodiments 141-186, wherein one of the first or second antigen binding domain is encoded by the nucleotide sequence set forth in SEQ ID NO:310.
   188. The polynucleotide of any of embodiments 141-187, wherein one of the first or second antigen binding domain is encoded by a nucleotide sequence set forth in SEQ ID NO:264 or SEQ ID NO: 311.
   189. The polynucleotide of any of embodiments 141-188, wherein the first or second antigen binding domain is encoded by the nucleotide sequence set forth in SEQ ID NO:310, and the other of the first or second antigen binding domain is encoded by the nucleotide sequence set forth in SEQ ID NO:311.
   190. The polynucleotide of any of embodiments 141-189, wherein (b) comprises a portion of an immunoglobulin.
   191. The polynucleotide of any of embodiments 141-190, wherein (b) comprises a sequence of a hinge region, a CH2 and CH3 region.
   192. The polynucleotide of embodiment 191, wherein:
the hinge region comprises all or a portion of an IgG4 hinge region and/or an IgG2 hinge region, wherein the IgG4 hinge region is optionally a human IgG4 hinge region and the IgG2 hinge region is optionally a human IgG2 hinge region;
the C_{H}2 region comprises all or a portion of an IgG4 C_{H}2 and/or an IgG2 C_{H}2, wherein the IgG4 C_{H}2 is optionally a human IgG4 C_{H}2 and the IgG2 C_{H}2 is optionally a human IgG2 C_{H}2; and/or
the C_{H}3 region comprises all or a portion of an IgG4 C_{H}3 and/or an IgG2 C_{H}3, wherein the IgG4 C_{H}3 is optionally a human IgG4 C_{H}3 and the IgG2 C_{H}3 is optionally a human IgG2 C_{H}3.
   193. The polynucleotide of embodiment 191 or embodiment 192, wherein the hinge region, C_{H}2 and C_{H}3 comprises all or a portion of a hinge, all or a portion of a C_{H}2 and all or a portion of a C_{H}3 from human IgG4.
   194. The polynucleotide of embodiment 191 or embodiment 193, wherein one or more of the hinge region, the C_{H}2 and the C_{H}3 is chimeric and comprises a hinge, C_{H}2 and C_{H}3 from human IgG4 and human IgG2.
   195. The polynucleotide of any of embodiments 141-194, (b) comprises a IgG4/2 chimeric hinge region or a modified IgG4 hinge region comprising at least one amino acid replacement compared to a human IgG4 hinge; an IgG2/4 chimeric C_{H}2 region; and an IgG4 C_{H}3 region.
   196. The polynucleotide of any of embodiments 141-195, wherein (b) has a length from or from about 125 to 300 amino acids in length, 125 to 250 amino acids in length, 125 to 230 amino acids in length, 125 to 200 amino acids in length, 125 to 180 amino acids in length, 125 to 150 amino acids in length, 150 to 300 amino acids in length, 150 to 250 amino acids in length, 150 to 230 amino acids in length, 150 to 200 amino acids in length, 150 to 180 amino acids in length, 180 to 300 amino acids in length, 180 to 250 amino acids in length, 180 to 230 amino acids in length, 180 to 200 amino acids in length, 200 to 300 amino acids in length, 200 to 250 amino acids in length, 200 to 230 amino acids in length, 230 to 300 amino acids in length, 230 to 250 amino acids in length or 250 to 300 amino acids in length, optionally wherein the spacer is at or about 224, at or about 225, at or about 226, at or about 227, at or about 228 or at or about 229 amino acids in length.
   197. The polynucleotide of any of embodiments 141-196, wherein (b) is or comprises the amino acid sequence set forth in SEQ ID NO:17.
   198. The polynucleotide of any of embodiments 141-197, wherein (b) in one of the first CAR or the second CAR is encoded by the nucleotide sequence set forth in SEQ ID NO:48 and (b) in the other of the first CAR or the second CAR is encoded by the nucleotide sequence set forth in SEQ ID NO:305.
   199. The polynucleotide of any of embodiments 141-198, wherein (c) is or comprises a transmembrane domain of CD4, CD28, or CD8, optionally a transmembrane domain from human CD4, human CD28 or human CD8.
   200. The polynucleotide of any of embodiments 141-199, wherein (c) is or comprises a human CD28 transmembrane domain.
   201. The polynucleotide of any of embodiments 141-200, wherein (c) is or comprises the amino acid sequence set forth in SEQ ID NO:18.
   202. The polynucleotide of embodiment 141-201, wherein (c) in one of the first CAR or the second CAR is encoded by the nucleotide sequence set forth in SEQ ID NOS:56 and (c) in the other of the first CAR or second CAR is encoded by the nucleotide sequence set forth in SEQ ID NO: 307.
   203. The polynucleotide of any of embodiments 141202, wherein the intracellular signaling domain of (d) is capable of inducing a primary activation signal in a T cell, is a T cell receptor (TCR) component and/or comprises an immunoreceptor tyrosine-based activation motif (ITAM).
   204. The polynucleotide of any of embodiments 141-203, wherein the intracellular signaling domain of (d) is or comprises a cytoplasmic signaling domain of a CD3-zeta (CD3ζ) chain or a functional variant or signaling portion thereof, optionally a human CD3 zeta chain.
   205. The polynucleotide of any of embodiments 141-204, wherein the intracellular signaling domain of (d) is or comprises the amino acid sequence set forth in SEQ ID NO:20.
   206. The polynucleotide of any of embodiments 141-205, wherein the intracellular signaling domain of (d) in one of the first CAR or the second CAR is encoded by the nucleotide sequence set forth in SEQ ID NO:58 and the intracellular signaling domain of (d) in the other of the first CAR or the second CAR is encoded by the nucleotide sequence set forth in SEQ ID NO:309.
   207. The polynucleotide of any of embodiments 141-206, wherein the costimulatory signaling region of (d) comprises an intracellular signaling domain of a T cell costimulatory molecule or a signaling portion thereof.
   208. The polynucleotide of any of embodiments 14120, wherein the costimulatory signaling region of (d) comprises an intracellular signaling domain of CD28, 4-1BB, or ICOS, or a signaling portion thereof, optionally of human CD28, human 4-1BB, or human ICOS.
   0209. The polynucleotide of any of embodiments 141-208, wherein the costimulatory signaling region of (d) comprises an intracellular signaling domain of 4-1BB.
   210. The polynucleotide of any of embodiments 141-209, wherein the costimulatory signaling region of (d) is or comprises the amino acid sequence set forth in SEQ ID NO:19.
   211. The polynucleotide of any of embodiments 141-210, wherein the costimulatory signaling region of (d) in one of the first CAR or the second CAR is encoded by the nucleotide sequence set forth in SEQ ID NOS:60 and the costimulatory signaling region of (d) in the other of the first CAR or the second CAR is encoded by the nucleotide sequence set forth in SEQ ID NO:308.
   212. The polynucleotide of any of embodiments 141-211, wherein:
one of the first CAR or the second CAR comprises (a) a first antigen binding domain that binds to GPRC5D, optionally wherein the first antigen binding domain is encoded by the nucleotide sequence set forth in SEQ ID NO:311; (b) a spacer encoded by the nucleotide set forth in SEQ ID NO:305, (c) a transmembrane domain encoded by the nucleotide sequence set forth in SEQ ID NO:307, and (d) an intracellular signaling region comprising an intracellular signaling domain encoded by the nucleotide sequence set forth in SEQ ID NO:309 and a co-stimulatory signaling region encoded by the nucleotide sequence set forth in SEQ ID NO:308;
the other of the first CAR or the second CAR comprises (a) an antigen binding domain that binds to BCMA, optionally wherein the antigen binding domain is encoded by the nucleotide sequence set forth in SEQ ID NO:310, (b) a spacer encoded by the nucleotide set forth in SEQ ID NO:48, (c) a transmembrane domain encoded by the nucleotide sequence set forth in SEQ ID NO:56, and (d) an intracellular signaling region comprising an intracellular signaling domain encoded by the nucleotide sequence set forth in SEQ ID NO:58 and a co-stimulatory signaling domain region encoded by the nucleotide sequence set forth in SEQ ID NO:60.
   213. The polynucleotide of any of embodiments 141-212, wherein the first nucleic acid sequence encoding the first CAR is located toward the 5′ end of the polynucleotide, relative to the second nucleic acid sequence encoding the first CAR.
   214. The polynucleotide of any of embodiments 141-213, wherein the first CAR comprises an antigen binding domain that binds to GPRC5D and the second CAR comprises an antigen binding domain that binds to BCMA.
   215. The polynucleotide of any of embodiments 141-213, wherein the first CAR comprises an antigen binding domain that binds to BCMA and the second CAR comprises an antigen binding domain that binds to GPRC5D.
   216. A polynucleotide comprising (i) a first nucleic acid sequence encoding a first chimeric antigen receptor (CAR), (ii) a second nucleic acid sequence encoding a second chimeric antigen receptor (CAR) and (iii) a nucleotide sequence encoding a multicistronic element, wherein the first nucleic acid encoding the first CAR and the second nucleic acid encoding the second CAR are separated the multicistronic element;
wherein the first CAR comprises a first antigen binding domain that binds to GPRC5D, optionally wherein the first antigen binding domain is encoded by the nucleotide sequence set forth in SEQ ID NO:311; a spacer encoded by the nucleotide set forth in SEQ ID NO:305; a transmembrane domain encoded by the nucleotide sequence set forth in SEQ ID NO:307; and an intracellular signaling region comprising an intracellular signaling domain encoded by the nucleotide sequence set forth in SEQ ID NO:309 and a co-stimulatory signaling region encoded by the nucleotide sequence set forth in SEQ ID NO:308;
wherein the second CAR comprises a second antigen binding domain that binds to BCMA optionally wherein the second antigen binding domain is encoded by the nucleotide sequence set forth in SEQ ID NO:310; a spacer encoded by the nucleotide set forth in SEQ ID NO:48; a transmembrane domain encoded by the nucleotide sequence set forth in SEQ ID NO:56; and an intracellular signaling region comprising an intracellular signaling domain encoded by the nucleotide sequence set forth in SEQ ID NO:58 and a co-stimulatory signaling domain region encoded by the nucleotide sequence set forth in SEQ ID NO:60;
and wherein the first nucleic acid sequence encoding the first CAR is located toward the 5′ end of the polynucleotide relative to the second nucleic acid sequence encoding the second CAR.
   217. A polynucleotide comprising (i) a first nucleic acid sequence encoding a first chimeric antigen receptor (CAR), (ii) a second nucleic acid sequence encoding a second chimeric antigen receptor (CAR), and (iii) a nucleotide sequence encoding a multicistronic element, wherein the first nucleic acid encoding the first CAR and the second nucleic acid encoding the second CAR are separated by the multicistronic element;
wherein the first CAR comprises a first antigen binding domain that binds to BCMA, optionally wherein the first antigen binding domain is encoded by the nucleotide sequence set forth in SEQ ID NO:310, a spacer encoded by the nucleotide set forth in SEQ ID NO:48, a transmembrane domain encoded by the nucleotide sequence set forth in SEQ ID NO:56, and an intracellular signaling region comprising an intracellular signaling domain encoded by the nucleotide sequence set forth in SEQ ID NO:58 and a co-stimulatory signaling domain region encoded by the nucleotide sequence set forth in SEQ ID NO:60
wherein the second CAR comprises a second antigen binding domain that binds to GPRC5D, optionally wherein the second antigen binding domain is encoded by the nucleotide sequence set forth in SEQ ID NO:311, a spacer encoded by the nucleotide set forth in SEQ ID NO:305, a transmembrane domain encoded by the nucleotide sequence set forth in SEQ ID NO:307, and an intracellular signaling region comprising an intracellular signaling domain encoded by the nucleotide sequence set forth in SEQ ID NO:309 and a co-stimulatory signaling region encoded by the nucleotide sequence set forth in SEQ ID NO:308;
wherein the first nucleic acid encoding the first CAR is located toward the 5′ end of the polynucleotide relative to the second nucleic acid sequence encoding the second CAR.
   218. The polynucleotide of any of embodiments 163-217, wherein the multicistronic element comprising the amino acid sequence set forth in SEQ ID NO:37.
   219. The polynucleotide of any of embodiments 163-218, wherein the multicistronic element is encoded by a nucleotide sequences set forth in SEQ ID NOS:44 or SEQ ID NO: 45.
   220. The polynucleotide of any of embodiments 141-75 and 217-219, comprising the nucleotide sequence set forth in SEQ ID NO:299.
   221. The polynucleotide of any of embodiments 141-215 and 217-219, wherein the polynucleotide encodes the sequence set forth in SEQ ID NO:298.
   222. The polynucleotide of any of embodiments 141-216, 218 and 219, comprising the nucleotide sequence set forth in SEQ ID NO:302.
   223. The polynucleotide of any of embodiments 141-216, 218, 219 and 222, wherein the polynucleotide encodes the sequence set forth in SEQ ID NO:301.
   224. A vector comprising the polynucleotide of any of embodiments 79-223.
   225. The vector of embodiment 224, which is a viral vector.
   226. The vector of embodiment 225, wherein the viral vector is a lentiviral vector or a retroviral vector.
   227. A cell comprising the chimeric antigen receptor of any of embodiments 1-78.
   228. The cell of embodiment 141, wherein the chimeric antigen receptor is a first chimeric receptor and the cell further comprises a polynucleotide comprising a nucleotide encoding a second chimeric antigen receptor.
   229. A cell comprising the polynucleotide of any of embodiments 79-223.
   230. A cell comprising the polynucleotide of any of embodiments 79-97, which is the first polynucleotide and wherein the cell further comprises a second polynucleotide comprising a nucleotide sequence encoding a second chimeric antigen receptor (CAR).
   231. The cell of embodiment 228 or embodiment 230, wherein the second chimeric antigen receptor (CAR) comprises an extracellular antigen-binding domain that specifically binds a second antigen expressed on or associated with multiple myeloma.
   232. The cell of embodiment 231, wherein the second CAR further comprises a spacer, a transmembrane domain, and an intracellular signaling region.
   233. The cell of embodiment 231 or embodiment 232, wherein the second antigen is selected from B cell maturation antigen (BCMA), CD38, CD138, CS-1, BAFF-R, TACI, and FcRH5.
   234. The cell of any of embodiments 231-233, wherein the second antigen is BCMA.
   235. The cell of any of embodiments 142-234, wherein the second CAR comprises:
(1) an extracellular antigen-binding domain that specifically binds BCMA, wherein the extracellular antigen-binding domain comprises:
   (i) a variable heavy chain (VH) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 189, 191, 193, 195 or 197; and
   (ii) a variable light chain (VL) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 190, 192, 194, 196 or 198;
(2) a spacer, optionally a spacer comprising an IgG4/2 chimeric hinge or a modified IgG4 hinge; an IgG2/4 chimeric CH2 region; and an IgG4 CH3 region, optionally that is at or about 228 amino acids in length, or a spacer set forth in SEQ ID NO:17;
(3) a transmembrane domain; and
(4) an intracellular signaling region.
   236. The cell of embodiment 235, wherein the VH region of the second CAR comprises a CDR-H1, CDR-H2 and CDR-H3 contained within the VH region amino acid sequence set forth in SEQ ID NO: 189, 191, 193, 195 or 197; and the VL region comprises a CDR-L1, CDR-L2 and CDR-L3 contained within the VL region amino acid sequence set forth in SEQ ID NO: 190, 192, 194, 196 or 198. 237. The cell of any of embodiments 231-234, wherein the second CAR comprises:
(1) an extracellular antigen-binding domain that specifically binds BCMA, wherein the extracellular antigen-binding domain comprises:
   (i) a variable heavy chain (VH) region comprising a heavy chain complementarity determining region 1 (CDR-H1) comprising the amino acid sequence set forth in SEQ ID NO: 199, 202, 206, 209, 212 or 215; (b) a heavy chain complementarity determining region 2 (CDR-H2) comprising the amino acid sequence set forth in SEQ ID NO: 200, 203, 207, 210, 213 or 216; and (c) a heavy chain complementarity determining region 3 (CDR-H3) comprising the amino acid sequence set forth in SEQ ID NO: 201, 204, 205, 208, 211, 214 or 217; and
   (ii) a variable light chain (VL) region comprising a light chain complementarity determining region 1 (CDR-L1) comprising the amino acid sequence set forth in SEQ ID NO: 218, 221, 224, 227, 230, 233 or 235; (b) a light chain complementarity determining region 2 (CDR-L2) comprising the amino acid sequence set forth in SEQ ID NO: 219, 222, 225, 228, 231, 234 or 236; and (c) a light chain complementarity determining region 3 (CDR-L3) comprising the amino acid sequence set forth in SEQ ID NO: 220, 223, 226, 229 or 232;
(2) a spacer, optionally a spacer comprising an IgG4/2 chimeric hinge or a modified IgG4 hinge; an IgG2/4 chimeric CH2 region; and an IgG4 CH3 region, optionally that is about 228 amino acids in length, or a spacer set forth in SEQ ID NO:17;
(3) a transmembrane domain; and
(4) an intracellular signaling region.
   238. The cell of any of embodiments 228-237, wherein:
the VH region of the second CAR comprises a CDR-H1, CDR-H2, and CDR-H3 comprising the amino acid sequence of SEQ ID NOS:199, 200 and 201, respectively, and the VL region of the second CAR comprises a CDR-L1, CDR-L2, and CDR-L3 comprising the amino acid sequence of SEQ ID NOS:218, 219 and 220, respectively;
the VH region of the second CAR comprises a CDR-H1, CDR-H2, and CDR-H3 comprising the amino acid sequence of SEQ ID NOS:202, 203, 204, respectively, and the VL region of the second CAR comprises a CDR-L1, CDR-L2, and CDR-L3 comprising the amino acid sequence of SEQ ID NOS:221, 222 and 223, respectively;
the VH region of the second CAR comprises a CDR-H1, CDR-H2, and CDR-H3 comprising the amino acid sequence of SEQ ID NOS:199, 200, 205, respectively, and the VL region of the second CAR comprises a CDR-L1, CDR-L2, and CDR-L3 comprising the amino acid sequence of SEQ ID NOS:224, 225, and 226, respectively;
the VH region of the second CAR comprises a CDR-H1, CDR-H2, and CDR-H3 comprising the amino acid sequence of SEQ ID NOS:206, 207, 208, respectively, and the VL region of the second CAR comprises a CDR-L1, CDR-L2, and CDR-L3 comprising the amino acid sequence of SEQ ID NOS:227, 228 and 229, respectively;
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively;
the VH region of the second CAR comprises a CDR-H1, CDR-H2, and CDR-H3 comprising the amino acid sequence of SEQ ID NOS:212, 213 and 214, respectively, and the VL region of the second CAR comprises a CDR-L1, CDR-L2, and CDR-L3 comprising the amino acid sequence of SEQ ID NOS:233, 234 and 229, respectively; or
the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively.
   239. The cell of any of embodiments 235-238, wherein:
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively; or
the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively.
   240. The cell of any of embodiments 235-239, wherein:
the VH region and VL region of the second CAR comprises (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the VH region and VL region of the second CAR comprises (a) the amino acid sequences set forth in SEQ ID NO:191 and SEQ ID NO:192, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the VH region and VL region of the second CAR comprises (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the VH region and VL region of the second CAR comprises (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the VH region and VL region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:198.
   250. The cell of any of embodiments 235-249, wherein:
the VH region and VL region of the second CAR comprise the amino acid sequences set forth in SEQ ID NO:189 and SEQ ID NO:190, respectively;
the VH region and VL region of the second CAR comprise the amino acid sequences set forth in SEQ ID NO:191 and SEQ ID NO:192;
the VH region and VL region of the second CAR comprise the amino acid sequences set forth in SEQ ID NO:193 and SEQ ID NO:194;
the VH region and VL region of the second CAR comprise the amino acid sequences set forth in SEQ ID NO:195 and SEQ ID NO:196; or
the VH region and VL region of the second CAR comprise the amino acid sequences set forth in SEQ ID NO:197 and SEQ ID NO:198, respectively.
   251. The cell of any of embodiments 231-250, wherein the extracellular antigen-binding domain of the second CAR is a single chain antibody fragment.
   252. The cell of embodiment 251, wherein the single chain antibody fragment is or comprises a single chain variable fragment (scFv).
   253. The cell of any of embodiments 235-252, when the VH region and the VL region of the second CAR are joined by a flexible linker.
   254. The cell of embodiment 253, wherein the linker of the second CAR comprises the amino acid sequence GGGGSGGGGSGGGGS (SEQ ID NO:52).
   255. The cell of any of embodiments 235-254, wherein the VH region is amino-terminal to the VL region in the second CAR.
   256. The cell of any of embodiments 235-254, wherein the VH region is carboxy-terminal to the VL region in the second CAR.
   257. The cell of any of embodiments 231-256, wherein the antigen-binding domain of the second CAR comprises the amino acid sequence selected from SEQ ID NOs: 237, 238, 239, 240, and 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 237, 238, 239, 240, and 241.
   258. The cell of any of embodiments 231-257, wherein the antigen-binding domain of the second CAR comprises the amino acid sequence selected from SEQ ID NOs: 237, 238, 239, 240, and 241.
   259. The cell of any of embodiments 235-258, wherein:
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively; or the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively; and/or
the VH region and VL region of the second CAR comprise the amino acid sequences set forth in SEQ ID NO:197 and SEQ ID NO:198, respectively; and/or
the antigen-binding domain of the second CAR comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.
   260. The cell of any of embodiments 231-259, wherein the transmembrane domain of the second CAR is or comprises a transmembrane domain from CD4, CD28, or CD8, optionally from human CD4, human CD38 or human CD8.
   260. The cell of any of embodiments 232-259, wherein:
the transmembrane domain of the second CAR is or comprises a transmembrane domain from human CD28; and/or
the transmembrane domain of the second CAR is or comprises the sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.
   261. The cell of embodiment 260, wherein the transmembrane domain of the second CAR is or comprises the sequence set forth in SEQ ID NO:18.
   262. The cell of any of embodiments 232-261, wherein the intracellular signaling region of the second CAR comprises an intracellular signaling domain.
   263. The cell of embodiment 262, wherein the intracellular signaling domain is capable of inducing a primary activation signal in a T cell, is a T cell receptor (TCR) component and/or comprises an immunoreceptor tyrosine-based activation motif (ITAM).
   264. The cell of embodiment 262 or embodiment 263, wherein the intracellular signaling domain is or comprises a cytoplasmic signaling domain of a CD3-zeta (CD3ζ) chain or a functional variant or signaling portion thereof, optionally a human CD3 zeta chain.
   265. The cell of any of embodiments 262-264, wherein the intracellular signaling region comprises the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.
   266. The cell of any of embodiments 262-265, wherein the intracellular signaling region further comprises a costimulatory signaling region.
   267. The cell of embodiment 266, wherein the costimulatory signaling region comprises an intracellular signaling domain of a T cell costimulatory molecule or a signaling portion thereof.
   268. The cell of embodiment 266 or embodiment 267, wherein the costimulatory signaling region comprises an intracellular signaling domain of CD28, 4-1BB, or ICOS, or a signaling portion thereof, optionally human CD28, human 4-1BB, or human ICOS.
   269. The cell of any of embodiments 266-268, wherein the costimulatory signaling region comprises:
an intracellular signaling domain of human CD28; and/or
the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.
   270. The cell of any of embodiments 266-269, wherein the costimulatory signaling region comprises:
an intracellular signaling domain of human 4-1BB; and/or
the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.
   271. The cell of any of embodiments 228-270, wherein the second chimeric antigen receptor comprises from its N to C terminus in order: the antigen-binding domain, the spacer, the transmembrane domain and the intracellular signaling region.
   272. The cell of any of embodiments 227-271, which is a lymphocyte.
   273. The cell of embodiment 272, which is an NK cell or a T cell.
   274. The cell of embodiment 272 or embodiment 273, wherein the cell is a T cell and the T cell is a CD4+ or a CD8+ T cell.
   275. The cell of any of embodiments 227-274, wherein the cell is a primary cell obtained from a subject.
   276. The cell of embodiments 227-275, wherein, among a plurality of the cells, less than at or about 10%, at or about 9%, at or about 8%, at or about 7%, at or about 5%, at or about 4%, at or about 3%, at or about 2% or at or about 1% of the cells in the plurality comprise a chimeric antigen receptor that exhibits tonic signaling and/or antigen independent activity or signaling.
   277. A composition comprising the chimeric antigen receptor of any of embodiments 1-78.
   278. A composition comprising the cell of any one of embodiments 227-277 or a plurality of the cells of any one of embodiments 227-277.
   279. The composition of embodiment 278, wherein the composition comprises CD4+ and CD8+ T cells and the ratio of CD4+ to CD8+ T cells is from about 1:3 to 3:1, optionally about 1:2 to 2:1.
   280. A composition comprising:
a plurality of first cells comprising a first chimeric antigen receptor that is the chimeric antigen receptor of any of embodiments 1-78 or encoded by the polynucleotide of any of embodiments 79-97; and
a plurality of second cells comprising a second chimeric antigen receptor.
   281. The composition of embodiment 280, wherein, among a plurality of the first cells, less than at or about 10%, at or about 9%, at or about 8%, at or about 7%, at or about 5%, at or about 4%, at or about 3%, at or about 2% or at or about 1% of the cells in the plurality comprise a chimeric antigen receptor that exhibits tonic signaling and/or antigen independent activity or signaling.
   282. The composition of embodiment 280 or embodiment 281, wherein, among a plurality of the second cells, less than at or about 10%, at or about 9%, at or about 8%, at or about 7%, at or about 5%, at or about 4%, at or about 3%, at or about 2% or at or about 1% of the cells in the plurality comprise a chimeric antigen receptor that exhibits tonic signaling and/or antigen independent activity or signaling.
   283. The composition of any of embodiments 280-282, wherein the second chimeric receptor comprises an extracellular antigen-binding domain that specifically binds a second antigen expressed on or associated with multiple myeloma.
   284. The composition of any of embodiments 280-283, wherein the second CAR comprises the extracellular antigen-binding domain that binds the second antigen, a spacer, a transmembrane domain, and an intracellular signaling region.
   285. The composition of embodiment 283 or embodiment 284, wherein the second antigen is selected from B cell maturation antigen (BCMA), CD38, CD138, CS-1, BAFF-R, TACI, and FcRH5.
   286. The composition of any of embodiments 280-285, wherein the second antigen is BCMA.
   287. The composition of any of embodiments 280-286, wherein the second CAR comprises:
(1) an extracellular antigen-binding domain that specifically binds BCMA, wherein the extracellular antigen-binding domain comprises:
   (i) a variable heavy chain (VH) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 189, 191, 193, 195 or 197; and
   (ii) a variable light chain (VL) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 190, 192, 194, 196 or 198;
(2) a spacer, optionally a spacer comprising an IgG4/2 chimeric hinge or a modified IgG4 hinge; an IgG2/4 chimeric CH2 region; and an IgG4 CH3 region, optionally that is about 228 amino acids in length, or a spacer set forth in SEQ ID NO:17;
(3) a transmembrane domain; and
(4) an intracellular signaling region.
   288. The composition of embodiment 287, wherein the VH region of the second CAR comprises a CDR-H1, CDR-H2 and CDR-H3 contained within the VH region amino acid sequence set forth in SEQ ID NO: 189, 191, 193, 195 or 197; and the VL region comprises a CDR-L1, CDR-L2 and CDR-L3 contained within the VL region amino acid sequence set forth in SEQ ID NO: 190, 192, 194, 196, or 198.
   289. The composition of any of embodiments 280-286, wherein the second CAR comprises:
(1) an extracellular antigen-binding domain that specifically binds BCMA, wherein the extracellular antigen-binding domain comprises:
   (i) a variable heavy chain (VH) region comprising a heavy chain complementarity determining region 1 (CDR-H1) comprising the amino acid sequence set forth in SEQ ID NO: 199, 202, 206, 209, 212 or 215; (b) a heavy chain complementarity determining region 2 (CDR-H2) comprising the amino acid sequence set forth in SEQ ID NO: 200, 203, 207, 210, 213 or 216; and (c) a heavy chain complementarity determining region 3 (CDR-H3) comprising the amino acid sequence set forth in SEQ ID NO: 201, 204, 205, 208, 211, 214 or 217; and
   (ii) a variable light chain (VL) region comprising a light chain complementarity determining region 1 (CDR-L1) comprising the amino acid sequence set forth in SEQ ID NO: 218, 221, 224, 227, 230, 233 or 235; (b) a light chain complementarity determining region 2 (CDR-L2) comprising the amino acid sequence set forth in SEQ ID NO: 219, 222, 225, 228, 231, 234 or 236; and (c) a light chain complementarity determining region 3 (CDR-L3) comprising the amino acid sequence set forth in SEQ ID NO: 220, 223, 226, 229 or 232;
(2) a spacer, optionally a spacer comprising an IgG4/2 chimeric hinge or a modified IgG4 hinge; an IgG2/4 chimeric CH2 region; and an IgG4 CH3 region, optionally that is about 228 amino acids in length, or a spacer set forth in SEQ ID NO:17;
(3) a transmembrane domain; and
(4) an intracellular signaling region.
   290. The composition of any of embodiments 286-289, wherein:
the VH region of the second CAR comprises a CDR-H1, CDR-H2, and CDR-H3 comprising the amino acid sequence of SEQ ID NOS:199, 200 and 201, respectively, and the VL region of the second CAR comprises a CDR-L1, CDR-L2, and CDR-L3 comprising the amino acid sequence of SEQ ID NOS:218, 219 and 220, respectively;
the VH region of the second CAR comprise a CDR-H1, CDR-H2, and CDR-H3 comprising the amino acid sequence of SEQ ID NOS:202, 203, 204, respectively, and the VL region of the second CAR comprises a CDR-L1, CDR-L2, and CDR-L3 comprising the amino acid sequence of SEQ ID NOS:221, 222 and 223, respectively;
the VH region of the second CAR comprises a CDR-H1, CDR-H2, and CDR-H3 comprising the amino acid sequence of SEQ ID NOS:199, 200, 205, respectively, and the VL region of the second CAR comprises a CDR-L1, CDR-L2, and CDR-L3 comprising the amino acid sequence of SEQ ID NOS:224, 225, and 226, respectively;
the VH region of the second CAR comprises a CDR-H1, CDR-H2, and CDR-H3 comprising the amino acid sequence of SEQ ID NOS:206, 207, 208, respectively, and the VL region of the second CAR comprises a CDR-L1, CDR-L2, and CDR-L3 comprising the amino acid sequence of SEQ ID NOS:227, 228 and 229, respectively;
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively;
the VH region of the second CAR comprises a CDR-H1, CDR-H2, and CDR-H3 comprising the amino acid sequence of SEQ ID NOS:212, 213 and 214, respectively, and the VL region of the second CAR comprises a CDR-L1, CDR-L2, and CDR-L3 comprising the amino acid sequence of SEQ ID NOS:233, 234 and 229, respectively; or
the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively.
   291. The composition of any of embodiments 286-290, wherein:
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively; or
the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively.
   292. The composition of any of embodiments 286-291, wherein:
the VH region and VL region of the second CAR comprise (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the VH region and VL region of the second CAR comprises (a) the amino acid sequences set forth in SEQ ID NO:191 and SEQ ID NO:192, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the VH region and VL region of the second CAR comprises (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the VH region and VL region of the second CAR comprises (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the VH region and VL region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:198.
   293. The composition of any of embodiments 286-292, wherein:
the VH region and VL region of the second CAR comprise the amino acid sequences set forth in SEQ ID NO:189 and SEQ ID NO:190, respectively;
the VH region and VL region of the second CAR comprise the amino acid sequences set forth in SEQ ID NO:191 and SEQ ID NO:192;
the VH region and VL region of the second CAR comprise the amino acid sequences set forth in SEQ ID NO:193 and SEQ ID NO:194;
the VH region and VL region of the second CAR comprise the amino acid sequences set forth in SEQ ID NO:195 and SEQ ID NO:196; or
the VH region and VL region of the second CAR comprise the amino acid sequences set forth in SEQ ID NO:197 and SEQ ID NO:198, respectively.
   294. The composition of any of embodiments 286-293, wherein the extracellular antigen-binding domain of the second CAR is a single chain antibody fragment.
   295. The composition of embodiment 294, wherein the single chain antibody fragment is or comprises a single chain variable fragment (scFv).
   296. The composition of any of embodiments 286-295, when the VH region and the VL region of the second CAR are joined by a flexible linker.
   297. The composition of embodiment 296, wherein the linker of the second CAR comprises the amino acid sequence GGGGSGGGGSGGGGS (SEQ ID NO:52).
   298. The composition of any of embodiments 286-297, wherein the VH region is amino-terminal to the VL region in the second CAR.
   299. The composition of any of embodiments 286-298, wherein the VH region is carboxy-terminal to the VL region in the second CAR.
   300. The composition of any of embodiments 286-299, wherein the antigen-binding domain of the second CAR comprises the amino acid sequence selected from SEQ ID NOs: 237, 238, 239, 240, and 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 237, 238, 239, 240, and 241.
   301. The composition of any of embodiments 386-300, wherein the antigen-binding domain of the second CAR comprises the amino acid sequence selected from SEQ ID NOs: 237, 238, 239, 240, and 241.
   302. The composition of any of embodiments 286-301, wherein:
the VH region of the second CAR comprises a CDR-H1, CDR-H2, and CDR-H3 comprising the amino acid sequence of SEQ ID NOS:209, 210 and 211, respectively, and the VL region of the second CAR comprises a CDR-L1, CDR-L2, and CDR-L3 comprising the amino acid sequence of SEQ ID NOS:230, 231 and 232, respectively; or the VH region of the second CAR comprises a CDR-H1, CDR-H2, and CDR-H3 comprising the amino acid sequence of SEQ ID NOS:215, 216 and 217, respectively, and the VL region of the second CAR comprises a CDR-L1, CDR-L2, and CDR-L3 comprising the amino acid sequence of SEQ ID NOS:235, 236 and 232, respectively; and/or
the VH region and VL region of the second CAR comprise the amino acid sequences set forth in SEQ ID NO:197 and SEQ ID NO:198, respectively; and/or
the antigen-binding domain of the second Car comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.
   303. The composition of any of embodiments 286-302, wherein the transmembrane domain of the second CAR is or comprises a transmembrane domain from CD4, CD28, or CD8, optionally from human CD4, human CD38 or human CD8.
   304. The composition of any of embodiments 286-303, wherein:
the transmembrane domain of the second CAR is or comprises a transmembrane domain from human CD28; and/or
the transmembrane domain of the second CAR is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.
   305. The composition of embodiment 304, wherein the transmembrane domain of the second CAR is or comprises the sequence set forth in SEQ ID NO:18.
   306. The composition of any of embodiments 286-305, wherein the intracellular signaling region of the second CAR comprises an intracellular signaling domain.
   307. The composition of embodiment 306, wherein the intracellular signaling domain of the second CAR is capable of inducing a primary activation signal in a T cell, is a T cell receptor (TCR) component and/or comprises an immunoreceptor tyrosine-based activation motif (ITAM).
   308. The composition of embodiment 306 or embodiment 307, wherein the intracellular signaling domain of the second CAR is or comprises a cytoplasmic signaling domain of a CD3-zeta (CD3ζ) chain or a functional variant or signaling portion thereof, optionally a human CD3 zeta chain.
   309. The composition of any of embodiments 306-308, wherein the intracellular signaling region of the second CAR comprises the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.
   310. The composition of any of embodiments 306-309, wherein the intracellular signaling region of the second CAR further comprises a costimulatory signaling region.
   311. The composition of embodiment 310, wherein the costimulatory signaling region of the second CAR comprises an intracellular signaling domain of a T cell costimulatory molecule or a signaling portion thereof.
   312. The composition of embodiment 310 or embodiment 311, wherein the costimulatory signaling region of the second CAR comprises an intracellular signaling domain of CD28, 4-1BB, or ICOS, or a signaling portion thereof, optionally human CD28, human 4-1BB, or human ICOS.
   313. The composition of any of embodiments 310-312, wherein the costimulatory signaling region of the second CAR comprises:
an intracellular signaling domain of human CD28; and/or
the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.
   314. The composition of any of embodiments 280-312, wherein at least one of the first chimeric antigen receptor and the second chimeric antigen receptor comprises an intracellular signaling region comprising an intracellular signaling domain of 4-1BB or a signaling portion thereof, optionally of human 4-1BB.
   315. The composition of any of embodiments 310-312, wherein the costimulatory signaling region of the second CAR comprises:
an intracellular signaling domain of human 4-1BB; and/or
the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.
   316. The composition of any of embodiments 280-315, wherein the encoded second chimeric antigen receptor comprises from its N to C terminus in order: the antigen-binding domain, the spacer, the transmembrane domain and the intracellular signaling region.
   317. The composition of any of embodiments 208-316, wherein the plurality of first cells comprises T cells, optionally wherein the T cells comprises CD4+ and CD8+ T cells, optionally wherein the ratio of CD4+ to CD8+ T cells is from about 1:3 to 3:1, optionally 1:2 to 2:1.
   318. The composition of any of embodiments 186-222, wherein the plurality of second cells comprises T cells, optionally wherein the T cells comprises CD4+ and CD8+ T cells, optionally wherein the ratio of CD4+ to CD8+ T cells is from about 1:3 to 3:1, optionally about 1:2 to 2:1.
   319. The composition of any of embodiments 280-318, wherein the composition comprises the ratio of the first plurality of cells and the second plurality of cells is from about 1:3 to 3:1, optionally about 1:2 to 2:1, optionally about 1:1.
   320. The composition of any of embodiments 280-319, wherein the composition comprises the first plurality of cells expressing the first chimeric antigen receptor and the second plurality of cells expressing the second chimeric antigen receptor at a ratio that is from about 1:3 to 3:1, optionally about 1:2 to 2:1, optionally about 1:1.
   321. The composition of any of embodiments 277-320, further comprising a pharmaceutically acceptable excipient.
   322. The composition of any of embodiments 277-321, which is sterile.
   323. A pharmaceutical composition for use in treating a disease or disorder, optionally a cancer, containing the cells of any of embodiments 227-276 as an active ingredient.
   324. A pharmaceutical composition for use treating a disease or disorder, optionally a cancer, containing the composition of any of embodiments 277-322 or 388 as an active ingredient.
   325. A pharmaceutical composition for use in treating a disease or disorder, optionally a cancer, containing a composition comprising a first dose of a plurality of first cells comprising a first chimeric antigen receptor that is the chimeric antigen receptor of any of embodiments 1-67 or encoded by the polynucleotide of any of embodiments 68-79 and a composition comprising a second dose of a plurality of second cells comprising a second chimeric antigen receptor as an active ingredient.
   326. A method of treatment, comprising administering a composition comprising a dose of cells of any of embodiments 79-97 or the composition of any of embodiments 280-325 and 388 to a subject having a disease or disorder.
   327. Use of the cells of any of embodiments 227-276 for treatment of a disease or disorder, optionally wherein the disease or condition is a cancer.
   328. Use of the composition of any of embodiments 277-322 or 388 for treatment of a disease or disorder, optionally wherein the disease or condition is a cancer.
   329. The use of the cells of any of embodiments 227-276 for the manufacture of a medicament for treatment of a disease or disorder, optionally wherein the disease or condition is a cancer.
   330. The use of the composition of any of embodiments 277-322, or 388 for the manufacture of a medicament for treatment of a disease or disorder, optionally wherein the disease or condition is a cancer.
   331. The method or use of any of embodiments 326-330 or the pharmaceutical composition for use of any of embodiments 323-325, wherein the dose of cells comprises between about 1.0×10⁷ CAR-expressing T cells and 1.2×10⁹ CAR-expressing T cells, between about 1.0×10⁷ CAR-expressing T cells and 6.5×10⁸ CAR-expressing T cells, between about 1.5×10⁷ CAR-expressing T cells and 6.5×10⁸ CAR-expressing T cells, between about 1.5×10⁷ CAR-expressing T cells and 6.0×10⁸ CAR-expressing T cells, between about 2.5×10⁷ CAR-expressing T cells and 6.0×10⁸ CAR-expressing T cells, between about 5.0×10⁷ CAR-expressing T cells and 6.0×10⁸ CAR-expressing T cells, between about 1.25×10⁷ CAR-expressing T cells and 1.2×10⁹ CAR-expressing T cells, between about 1.5×10⁷ CAR-expressing T cells and 1.2×10⁹ CAR-expressing T cells, between about 5.0×10⁷ CAR-expressing T cells and 4.5×10⁸ CAR-expressing T cells, or between about 1.5×10⁸ CAR-expressing T cells and 3.0×10⁸ CAR-expressing T cells, each inclusive.
   331. The method or use of any of embodiments 326-330 or the pharmaceutical composition for use of any of embodiments 323-325, wherein the dose of cells comprises at or about 1.5×10⁷, at or about 2.5×10⁷, at or about 5.0×10⁷, at or about 7.5×10⁷, at or about 1.5×10⁸, at or about 2.25×10⁸, at or about 3.0×10⁸, at or about 4.5×10⁸, at or about 6.0×10⁸, at or about 8.0×10⁸, or at or about 1.2×10⁹ CAR-expressing T cells.
   332. Use of a composition comprising a first dose of a plurality of first cells comprising a first chimeric antigen receptor that is the chimeric antigen receptor of any of embodiments 1-67 or encoded by the polynucleotide of any of embodiments 68-79 and a composition comprising a second dose of a plurality of second cells comprising a second chimeric antigen receptor for treatment of a disease or disorder, optionally wherein the disease or condition is a cancer.
   333. The use of a composition comprising a first dose of a plurality of first cells comprising a first chimeric antigen receptor that is the chimeric antigen receptor of any of embodiments 1-67 or encoded by the polynucleotide of any of embodiments 68-79 and a composition comprising a second dose of a plurality of second cells comprising a second chimeric antigen receptor for the manufacture of a medicament for treatment of a disease or disorder, optionally wherein the disease or condition is a cancer.
   334. A method of treatment, comprising:
administering a composition comprising a first dose of a plurality of first cells comprising a first chimeric antigen receptor that is the chimeric antigen receptor of any of embodiments 1-78 or encoded by the polynucleotide of any of embodiments 79-97 to a subject having a disease or disorder; and
administering to the subject a second dose of a composition comprising a plurality of second cells comprising a second chimeric antigen receptor.
   335. The method or use of embodiment 334 wherein the first dose of the plurality of first cells and the second dose of the plurality of second cells independently comprise between at or about 1.0×10⁷ CAR-expressing T cells and at or about 1.5×10⁹ CAR-expressing T cells, between at or about about 1.0×10⁷ CAR-expressing T cells and at or about 6.5×10⁸ CAR-expressing T cells, between at or about 1.25×10⁷ CAR-expressing T cells and at or about 0.6×10⁸ CAR-expressing T cells, between at or about 1.5×10⁷ CAR-expressing T cells and at or about 6.5×10⁸ CAR-expressing T cells, between at or about 1.5×10⁷ CAR-expressing T cells and at or about 6.0×10⁸ CAR-expressing T cells, between at or about 2.5×10⁷ CAR-expressing T cells and at or about 2.25×10⁸ CAR-expressing T cells, between at or about 2.5×10⁷ CAR-expressing T cells and at or about 6.0×10⁸ CAR-expressing T cells, between at or about 5.0×10⁷ CAR-expressing T cells and at or about 6.0×10⁸ CAR-expressing T cells, between at or about 7.5×10⁷ CAR-expressing T cells and at or about 1.5×10⁸ CAR-expressing T cells, between at or about 2.5×10⁷ CAR-expressing T cells and at or about 1.2×10⁹ CAR-expressing T cells, between at or about 5.0×10⁷ CAR-expressing T cells and at or about 4.5×10⁸ CAR-expressing T cells, or between at or about 1.5×10⁸ CAR-expressing T cells and at or about 3.0×10⁸ CAR-expressing T cells, each inclusive.
   336. The method or use of any of embodiment 334 or 335 or the pharmaceutical composition of any of embodiments 323-325, wherein the composition comprising the plurality of first cells and the composition comprising the plurality of second cells are administered simultaneously, sequentially or intermittently.
   337. The method of any of embodiments 334-336, wherein the composition comprising the plurality of first cells and the composition comprising the plurality of second cells are administered sequentially in any order.
   338. The method of any of embodiments 334-337, wherein, among a plurality of the first cells, less than at or about 10%, at or about 9%, at or about 8%, at or about 7%, at or about 5%, at or about 4%, at or about 3%, at or about 2% or at or about 1% of the cells in the plurality comprise a chimeric antigen receptor that exhibits tonic signaling and/or antigen independent activity or signaling.
   339 The method of any of embodiments 334-338, wherein, among a plurality of the second cells, less than at or about 10%, at or about 9%, at or about 8%, at or about 7%, at or about 5%, at or about 4%, at or about 3%, at or about 2% or at or about 1% of the cells in the plurality comprise a chimeric antigen receptor that exhibits tonic signaling and/or antigen independent activity or signaling.
   340. The method of any of embodiments 334-339, wherein the second chimeric receptor comprises an extracellular antigen binding domain that specifically binds a second antigen expressed on or associated with multiple myeloma.
   341. The method of any of embodiments 334-339, wherein the second CAR comprises the extracellular antigen binding domain that binds the second antigen, a spacer, a transmembrane domain, and an intracellular signaling region.
   342. The method of embodiment 340 or embodiment 341, wherein the second antigen is selected from B cell maturation antigen (BCMA), CD38, CD138, CS-1, BAFF-R, TACI, and FcRH5.
   343. The method of any of embodiments 334-342, wherein the second antigen is BCMA.
   344. The method of any of embodiments 334-343, wherein the second CAR comprises:
(1) an extracellular antigen-binding domain that specifically binds BCMA, wherein the extracellular antigen-binding domain comprises:
   (i) a variable heavy chain (VH) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 189, 191, 193, 195 or 197; and
   (ii) a variable light chain (VL) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 190, 192, 194, 196 or 198;
(2) a spacer, optionally a spacer comprising an IgG4/2 chimeric hinge or a modified IgG4 hinge; an IgG2/4 chimeric CH2 region; and an IgG4 CH3 region, optionally that is about 228 amino acids in length, or a spacer set forth in SEQ ID NO:17;
(3) a transmembrane domain; and
(4) an intracellular signaling region.
   345. The method of embodiment 344, wherein the VH region comprises a CDR-H1, CDR-H2 and CDR-H3 contained within the VH region amino acid sequence set forth in SEQ ID NO: 189, 191, 193, 195 or 197; and the VL region comprises a CDR-L1, CDR-L2 and CDR-L3 contained within the VL region amino acid sequence set forth in SEQ ID NO: 190, 192, 194, 196 or 198.
   346. The method of any of embodiments 344 or 345, wherein the second CAR comprises:
(1) an extracellular antigen-binding domain that specifically binds BCMA, wherein the extracellular antigen-binding domain comprises:
   (i) a variable heavy chain (VH) region comprising a heavy chain complementarity determining region 1 (CDR-H1) comprising the amino acid sequence selected from SEQ ID NOs: 199, 202, 206, 209, 212, and 215; (b) a heavy chain complementarity determining region 2 (CDR-H2) comprising the amino acid sequence selected from SEQ ID NOs: 200, 203, 207, 210, 213, and 216; and (c) a heavy chain complementarity determining region 3 (CDR-H3) comprising the amino acid sequence selected from SEQ ID NOs: 201, 204, 205, 208, 211, 214, and 217; and
   (ii) a variable light chain (VL) region comprising a light chain complementarity determining region 1 (CDR-L1) comprising the amino acid sequence selected from SEQ ID NOs: 218, 221, 224, 227, 230, 233, and 235; (b) a light chain complementarity determining region 2 (CDR-L2) comprising the amino acid sequence selected from SEQ ID NOs: 219, 222, 225, 228, 231, 234, and 236; and (c) a light chain complementarity determining region 3 (CDR-L3) comprising the amino acid sequence selected from SEQ ID NOs: 220, 223, 226, 229, and 232;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length, or a spacer set forth in SEQ ID NO:17;
(3) a transmembrane domain; and
(4) an intracellular signaling region.
   347. The method of any of embodiments 344-346, wherein:
the VH region of the second CAR comprises a CDR-H1, CDR-H2, and CDR-H3 comprising the amino acid sequences of SEQ ID NOS:199, 200 and 201, respectively, and the VL region of the second CAR comprises a CDR-L1, CDR-L2, and CDR-L3 comprising the amino acid sequences of SEQ ID NOS:218, 219 and 220, respectively;
the VH region of the second CAR comprises a CDR-H1, CDR-H2, and CDR-H3 comprising the amino acid sequences of SEQ ID NOS:202, 203, 204, respectively, and the VL region of the second CAR comprises a CDR-L1, CDR-L2, and CDR-L3 comprising the amino acid sequences of SEQ ID NOS:221, 222 and 223, respectively;
the VH region of the second CAR comprises a CDR-H1, CDR-H2, and CDR-H3 comprising the amino acid sequences of SEQ ID NOS:199, 200, 205, respectively, and the VL region of the second CAR comprises a CDR-L1, CDR-L2, and CDR-L3 comprising the amino acid sequences of SEQ ID NOS:224, 225, and 226, respectively;
the VH region of the second CAR comprises a CDR-H1, CDR-H2, and CDR-H3 comprising the amino acid sequences of SEQ ID NOS:206, 207, 208, respectively, and the VL region of the second CAR comprises a CDR-L1, CDR-L2, and CDR-L3 comprising the amino acid sequences of SEQ ID NOS:227, 228 and 229, respectively;
the VH region of the second CAR comprises a CDR-H1, CDR-H2, and CDR-H3 comprising the amino acid sequences of SEQ ID NOS:209, 210 and 211, respectively, and the VL region of the second CAR comprises a CDR-L1, CDR-L2, and CDR-L3 comprising the amino acid sequences of SEQ ID NOS:230, 231 and 232, respectively;
the VH region of the second CAR comprises a CDR-H1, CDR-H2, and CDR-H3 comprising the amino acid sequences of SEQ ID NOS:212, 213 and 214, respectively, and the VL region of the second CAR comprises a CDR-L1, CDR-L2, and CDR-L3 comprising the amino acid sequences of SEQ ID NOS:233, 234 and 229, respectively; or
the VH region of the second CAR comprises a CDR-H1, CDR-H2, and CDR-H3 comprising the amino acid sequences of SEQ ID NOS:215, 216 and 217, respectively, and the VL region of the second CAR comprises a CDR-L1, CDR-L2, and CDR-L3 comprising the amino acid sequences of SEQ ID NOS:235, 236 and 232, respectively.
   347. The method of any of embodiments 344-346, wherein:
the VH region of the second CAR comprises a CDR-H1, CDR-H2, and CDR-H3 comprising the amino acid sequences of SEQ ID NOS:209, 210 and 211, respectively, and the VL region of the second CAR comprises a CDR-L1, CDR-L2, and CDR-L3 comprising the amino acid sequences of SEQ ID NOS:230, 231 and 232, respectively; or
the VH region of the second CAR comprises a CDR-H1, CDR-H2, and CDR-H3 comprising the amino acid sequences of SEQ ID NOS:215, 216 and 217, respectively, and the VL region of the second CAR comprises a CDR-L1, CDR-L2, and CDR-L3 comprising the amino acid sequences of SEQ ID NOS:235, 236 and 232, respectively.
   348. The method of any of embodiments 344-347, wherein:
the VH region and VL region of the second CAR comprise (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the VH region and VL region of the second CAR comprise (a) the amino acid sequences set forth in SEQ ID NO:191 and SEQ ID NO:192, respectively, or (b) an amino acid sequence having a at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the VH region and VL region of the second CAR comprise (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the VH region and VL region of the second CAR comprises (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the VH region and VL region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and SEQ ID NO:198.
   349. The method of any of embodiments 344-348, wherein:
the VH region and VL region of the second CAR comprise the amino acid sequences set forth in SEQ ID NO:189 and SEQ ID NO:190, respectively;
the VH region and VL region of the second CAR comprise the amino acid sequences set forth in SEQ ID NO:191 and SEQ ID NO:192;
the VH region and VL region of the second CAR comprise the amino acid sequences set forth in SEQ ID NO:193 and SEQ ID NO:194;
the VH region and VL region of the second CAR comprise the amino acid sequences set forth in SEQ ID NO:195 and SEQ ID NO:196; or
the VH region and VL region of the second CAR comprise the amino acid sequences set forth in SEQ ID NO:197 and SEQ ID NO:198, respectively.
   350. The method of any of embodiments 344-349, wherein the extracellular antigen-binding domain of the 351. The method of embodiment 350, wherein the single chain antibody fragment is or comprises a single chain variable fragment (scFv).
   352. The method of any of embodiments 344-351, when the VH region and the VL region are joined by a flexible linker.
   353. The method of embodiment 352, wherein the linker comprises the amino acid sequence GGGGSGGGGSGGGGS (SEQ ID NO:52).
   354. The method of any of embodiments 344-353, wherein the VH region is amino-terminal to the VL region.
   355. The method of any of embodiments 344-354, wherein the VH region is carboxy-terminal to the VL region.
   356. The method of any of embodiments 344-355, wherein the antigen-binding domain comprises the amino acid sequence selected from SEQ ID NOs: 237, 238, 239, 240, and 241 or an amino acid sequence having a at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 237, 238, 239, 240, and 241.
   357. The method of any of embodiments 344-356, wherein the antigen-binding domain comprises the amino acid sequence selected from SEQ ID NOs: 237, 238, 239, 240, and 241.
   358. The method of any of embodiments 344-357, wherein:
the VH region of the second CAR comprises a CDR-H1, CDR-H2, and CDR-H3 comprising the amino acid sequences of SEQ ID NOS:209, 210 and 211, respectively, and the VL region of the second CAR comprises a CDR-L1, CDR-L2, and CDR-L3 comprising the amino acid sequences of SEQ ID NOS:230, 231 and 232, respectively; or the VH region of the second CAR comprises a CDR-H1, CDR-H2, and CDR-H3 comprising the amino acid sequences of SEQ ID NOS:215, 216 and 217, respectively, and the VL region of the second CAR comprises a CDR-L1, CDR-L2, and CDR-L3 comprising the amino acid sequences of SEQ ID NOS:235, 236 and 232, respectively; and/or
the VH region and VL region of the second CAR comprise the amino acid sequences set forth in SEQ ID NO:197 and SEQ ID NO:198, respectively; and/or
the antigen-binding domain comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.
   359. The method of any of embodiments 344-358, wherein the transmembrane domain is or comprises a transmembrane domain from CD4, CD28, or CD8, optionally from human CD4, human CD38 or human CD8.
   360. The method of any of embodiments 344-359, wherein:
the transmembrane domain is or comprises a transmembrane domain from human CD28; and/or
the transmembrane domain is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.
   361. The method of embodiment 360, wherein the transmembrane domain is or comprises the sequence set forth in SEQ ID NO:18.
   362. The method of any of embodiments 344-361, wherein the intracellular signaling region comprises an intracellular signaling domain.
   363. The method of embodiment 362, wherein the intracellular signaling domain is capable of inducing a primary activation signal in a T cell, is a T cell receptor (TCR) component and/or comprises an immunoreceptor tyrosine-based activation motif (ITAM).
   364. The method of embodiment 362 or embodiment 363, wherein the intracellular signaling domain is or comprises a cytoplasmic signaling domain of a CD3-zeta (CD3ζ) chain or a functional variant or signaling portion thereof, optionally a human CD3 zeta chain.
   365. The method or use of any of embodiments 362-364, wherein the intracellular signaling region comprises the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.
   366. The method or use of any of embodiments 362-365, wherein the intracellular signaling region further comprises a costimulatory signaling region.
   367. The method or use of embodiment 366, wherein the costimulatory signaling region comprises an intracellular signaling domain of a T cell costimulatory molecule or a signaling portion thereof.
   368. The method or use of embodiment 366 or embodiment 367, wherein the costimulatory signaling region comprises an intracellular signaling domain of CD28, 4-1BB, or ICOS or a signaling portion thereof, optionally human CD28, human 4-1BB, or human ICOS.
   369. The method or use of any of embodiments 366-368, wherein the costimulatory signaling region comprises:
an intracellular signaling domain of human CD28; and/or
the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.
   370. The method or use of any of embodiments 366-369, wherein the costimulatory signaling region comprises:
an intracellular signaling domain of a human 4-1BB; and/or
the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.
   371. The method or use of any of embodiments 344-370, wherein the encoded second chimeric antigen receptor comprises from its N to C terminus in order: the antigen-binding domain, the spacer, the transmembrane.
   372. The method or use of any of embodiments 326-371, wherein the disease or disorder is associated with expression of GPRC5D.
   373. The method or use of embodiment 372, wherein the disease or disorder is further associated with expression of B cell maturation antigen (BCMA).
   374. The method or use of any of embodiments 326-373, wherein the disease or disorder is a B cell-related disorder.
   375. The method or use of any one of embodiments 326-374, wherein the disease or disorder associated with BCMA is an autoimmune disease or disorder.
   376. The method or use of embodiment 375, wherein the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.
   377. The method or use of any one of embodiments 326-376, wherein the disease or disorder is a cancer.
   378. The method or use of embodiment 377, wherein the cancer is a GPRC5D-expressing cancer.
   379. The method or use of embodiment 377 or embodiment 378, wherein the cancer is a plasma cell malignancy and the plasma cell malignancy is multiple myeloma (MM) or plasmacytoma.
   380. The method or use of any of any of embodiments 377-379, wherein the cancer is multiple myeloma (MM).
   381. The method or use of embodiment 380, wherein the cancer is a relapsed/refractory multiple myeloma.
   382. The method or use of any of embodiments 326-381, wherein:
the subject is refractory to or has relapsed following administration of a BCMA-targeted therapy, optionally following administration of T cells comprising a CAR that specifically binds BCMA; or
the method comprises selecting a subject for treatment that is refractory to or has relapsed following administration of a BCMA-targeted therapy, optionally following administration T cells comprising a CAR that specifically binds BCMA.
   383. The method or use of any of embodiments 326-333 and 335-382, wherein prior to the administration of the dose of cells, the subject has previously received administration of a BCMA-targeted therapy for treating the disease or disorder.
   384. The method of any of embodiments 334, wherein prior to the administration of the first dose of cells and the second dose of cells, the subject has previously received administration of a BCMA-targeted therapy for treating the disease or disorder.
   385. The method or use of embodiment 383 or embodiment 383, wherein the BCMA-targeted therapy comprises a composition comprising T cells comprising a CAR that specifically binds BCMA.
   386. The method or use of any of embodiments 383-385, wherein the subject is refractory to or has relapsed following administration of the BCMA-targeted therapy, optionally following administration of T cells comprising a CAR that specifically binds BCMA.
   387. The method or use of any of embodiments 326-386, wherein the subject comprises multiple myeloma cells exhibiting BCMA antigen or epitope loss, BCMA downregulation and/or BCMA-negative tumor cells following a previous administration.
   388. The composition of embodiment 278 or embodiment 279, wherein the composition comprises a plurality of cells, wherein at least a portion of the cells comprise the first CAR that specifically binds GPRC5D, a portion of the cells comprise a second CAR that specifically binds a second antigen that is expressed on or associated with multiple myeloma, optionally wherein the second antigen is BCMA, and a portion of the cells comprise both the first CAR and the second CAR.
   389. A combination comprising:
a plurality of first cells comprising a first chimeric antigen receptor that is the chimeric antigen receptor of any of embodiments 1-78 and/or encoded by the polynucleotide of any of embodiments 79-97; and
a plurality of second cells comprising a second chimeric antigen receptor.
   390. The combination of embodiment 389, wherein, among a plurality of the first cells, less than about 10%, 9%, 8%, 7%, 5%, 4%, 3%, 2% or 1% of the cells in the plurality comprise a chimeric antigen receptor that exhibits tonic signaling and/or antigen independent activity or signaling.
   391. The combination of embodiment 389 or embodiment 390, wherein, among a plurality of the second cells, less than at or about 10%, at or about 9%, at or about 8%, at or about 7%, at or about 5%, at or about 4%, at or about 3%, at or about 2% or at or about 1% of the cells in the plurality comprise a chimeric antigen receptor that exhibits tonic signaling and/or antigen independent activity or signaling.
   392. The combination of any of embodiments 389-391, wherein the second chimeric receptor comprises an extracellular antigen binding domain that specifically binds a second antigen expressed on or associated with multiple myeloma.
   393. The combination of any of embodiments 389-391, wherein the second CAR comprises the extracellular antigen binding domain that binds the second antigen, a spacer, a transmembrane domain, and an intracellular signaling region.
   394. The combination of embodiment 392 or embodiment 393, wherein the second antigen is selected from the group consisting of B cell maturation antigen (BCMA), CD38, CD138, CS-1, BAFF-R, TACI, and FcRH5.
   395. The combination of any of embodiments 389-394, wherein the second antigen is BCMA.
   396. The combination of any of embodiments 389-395, wherein the second CAR comprises:
(1) an extracellular antigen-binding domain that specifically binds BCMA, wherein the extracellular antigen-binding domain comprises:
   (i) a variable heavy chain (VH) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 189, 191, 193, 195 or 197; and
   (ii) a variable light chain (VL) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in any of SEQ ID NO: 190, 192, 194, 196 or 198;
(2) a spacer, optionally a spacer comprising an IgG4/2 chimeric hinge or a modified IgG4 hinge; an IgG2/4 chimeric CH2 region; and an IgG4 CH3 region, optionally that is about 228 amino acids in length and/or a spacer set forth in SEQ ID NO:17;
(3) a transmembrane domain; and
(4) an intracellular signaling region.
   397. The combination of embodiment 396, wherein the VH region comprises a CDR-H1, CDR-H2 and CDR-H3 contained within the VH region amino acid sequence set forth in SEQ ID NO: 189, 191, 193, 195 or 197; and the VL region comprises a CDR-L1, CDR-L2 and CDR-L3 contained within the VL region amino acid sequence set forth in SEQ ID NO: 190, 192, 194, 196 or 198.
   398. The combination of any of embodiments 396 or 397, wherein the second CAR comprises:
(1) an extracellular antigen-binding domain that specifically binds BCMA, wherein the extracellular antigen-binding domain comprises:
   (i) a variable heavy chain (VH) comprising a heavy chain complementarity determining region 1 (CDR-H1) comprising the amino acid sequence set forth in SEQ ID NO: 199, 202, 206, 209, 212, or 215; (b) a heavy chain complementarity determining region 2 (CDR-H2) comprising the amino acid sequence set forth in SEQ ID NO: 200, 203, 207, 210, 213, or 216; and (c) a heavy chain complementarity determining region 3 (CDR-H3) comprising the amino acid sequence set forth in SEQ ID NOs: 201, 204, 205, 208, 211, 214, or 217; and
   (ii) a variable light chain (VL) region comprising a light chain complementarity determining region 1 (CDR-L1) comprising the amino acid sequence set forth in SEQ ID NOs: 218, 221, 224, 227, 230, 233, or 235; (b) a light chain complementarity determining region 2 (CDR-L2) comprising the amino acid sequence set forth in SEQ ID NOs: 219, 222, 225, 228, 231, 234, or 236; and (c) a light chain complementarity determining region 3 (CDR-L3) comprising the amino acid sequence set forth in SEQ ID NOs: 220, 223, 226, 229, or 232;
(2) a spacer, optionally a spacer comprising an IgG4/2 chimeric hinge or a modified IgG4 hinge; an IgG2/4 chimeric CH2 region; and an IgG4 CH3 region, optionally that is about 228 amino acids in length and/or a spacer set forth in SEQ ID NO:17;
(3) a transmembrane domain; and
(4) an intracellular signaling region.
   399. The combination of any of embodiments 396-398 wherein:
the VH region of the second CAR comprises a CDR-H1, CDR-H2, and CDR-H3 comprising the amino acid sequences of SEQ ID NOS:199, 200 and 201, respectively, and the VL region of the second CAR comprises a CDR-L1, CDR-L2, and CDR-L3 comprising the amino acid sequences of SEQ ID NOS:218, 219 and 220, respectively;
the VH region of the second CAR comprises a CDR-H1, CDR-H2, and CDR-H3 comprising the amino acid sequence of SEQ ID NOS:202, 203, 204, respectively, and the VL region of the second CAR comprises a CDR-L1, CDR-L2, and CDR-L3 comprising the amino acid sequence of SEQ ID NOS:221, 222 and 223, respectively;
the VH region of the second CAR comprises a CDR-H1, CDR-H2, and CDR-H3 comprising the amino acid sequences of SEQ ID NOS:199, 200, 205, respectively, and the VL region of the second CAR comprises a CDR-L1, CDR-L2, and CDR-L3 comprising the amino acid sequences of SEQ ID NOS:224, 225, and 226, respectively;
the VH region of the second CAR comprises a CDR-H1, CDR-H2, and CDR-H3 comprising the amino acid sequences of SEQ ID NOS:206, 207, 208, respectively, and the VL region of the second CAR comprises a CDR-L1, CDR-L2, and CDR-L3 comprising the amino acid sequences of SEQ ID NOS:227, 228 and 229, respectively;
the VH region of the second CAR comprises a CDR-H1, CDR-H2, and CDR-H3 comprising the amino acid sequences of SEQ ID NOS:209, 210 and 211, respectively, and the VL region of the second CAR comprises a CDR-L1, CDR-L2, and CDR-L3 comprising the amino acid sequences of SEQ ID NOS:230, 231 and 232, respectively;
the VH region of the second CAR comprises a CDR-H1, CDR-H2, and CDR-H3 comprising the amino acid sequences of SEQ ID NOS:212, 213 and 214, respectively, and the VL region of the second CAR comprises a CDR-L1, CDR-L2, and CDR-L3 comprising the amino acid sequences of SEQ ID NOS:233, 234 and 229, respectively; or
the VH region of the second CAR comprises a CDR-H1, CDR-H2, and CDR-H3 comprising the amino acid sequences of SEQ ID NOS:215, 216 and 217, respectively, and the VL region of the second CAR comprises a CDR-L1, CDR-L2, and CDR-L3 comprising the amino acid sequences of SEQ ID NOS:235, 236 and 232, respectively.
   400. The combination of any of embodiments 396-399, wherein:
the VH region of the second CAR comprises a CDR-H1, CDR-H2, and CDR-H3 comprising the amino acid sequences of SEQ ID NOS:209, 210 and 211, respectively, and the VL region of the second CAR comprises a CDR-L1, CDR-L2, and CDR-L3 comprising the amino acid sequences of SEQ ID NOS:230, 231 and 232, respectively; or
the VH region of the second CAR comprises a CDR-H1, CDR-H2, and CDR-H3 comprising the amino acid sequences of SEQ ID NOS:215, 216 and 217, respectively, and the VL region of the second CAR comprises a CDR-L1, CDR-L2, and CDR-L3 comprising the amino acid sequences of SEQ ID NOS:235, 236 and 232, respectively.
   401. The combination of any of embodiments 396-400, wherein:
the VH region and VL region of the second CAR comprise (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, at or about 99%, or at or about 100% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the VH region and VL region of the second CAR comprise (a) the amino acid sequences set forth in SEQ ID NO:191 and SEQ ID NO:192, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the VH region and VL region of the second CAR comprises (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the VH region and VL region of the second CAR comprises (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the VH region and VL region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:198.
   402. The combination of any of embodiments 396-401, wherein:
the VH region and VL region of the second CAR comprise the amino acid sequences set forth in SEQ ID NO:189 and SEQ ID NO:190, respectively;
the VH region and VL region of the second CAR comprise the amino acid sequences set forth in SEQ ID NO:191 and SEQ ID NO:192;
the VH region and VL region of the second CAR comprise the amino acid sequences set forth in SEQ ID NO:193 and SEQ ID NO:194;
the VH region and VL region of the second CAR comprise the amino acid sequences set forth in SEQ ID NO:195 and SEQ ID NO:196; or
the VH region and VL region of the second CAR comprise the amino acid sequences set forth in SEQ ID NO:197 and SEQ ID NO:198, respectively.
   403. The combination of any of embodiments 396-402, wherein the extracellular antigen-binding domain of the second CAR is a single chain antibody fragment.
   404. The combination of embodiment 403, wherein the single chain antibody fragment is or comprises a single chain variable fragment (scFv).
   405. The combination of any of embodiments 396-404, when the VH region and the VL region are joined by a flexible linker.
   406. The combination of embodiment 3405, wherein the linker comprises the amino acid sequence GGGGSGGGGSGGGGS (SEQ ID NO:52).
   407. The combination of any of embodiments 396-406, wherein the VH region is amino-terminal to the VL region.
   408. The combination of any of embodiments 396-406, wherein the VH region is carboxy-terminal to the VL 409. The combination of any of embodiments 396-408, wherein the antigen-binding domain comprises the amino acid sequence set forth in SEQ ID NOs: 237, 238, 239, 240, or 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 227, 238, 239, 240, or 241.
   410. The combination of any of embodiments 396-409, wherein the antigen-binding domain comprises the amino acid sequence set forth in SEQ ID NOs: 237, 238, 239, 240, or 241.
   411. The combination of any of embodiments 396-410, wherein:
the VH region of the second CAR comprises a CDR-H1, CDR-H2, and CDR-H3 comprising the amino acid sequences of SEQ ID NOS:209, 210 and 211, respectively, and the VL region of the second CAR comprises a CDR-L1, CDR-L2, and CDR-L3 comprising the amino acid sequences of SEQ ID NOS:230, 231 and 232, respectively; or the VH region of the second CAR comprises a CDR-H1, CDR-H2, and CDR-H3 comprising the amino acid sequences of SEQ ID NOS:215, 216 and 217, respectively, and the VL region of the second CAR comprises a CDR-L1, CDR-L2, and CDR-L3 comprising the amino acid sequences of SEQ ID NOS:235, 236 and 232, respectively; and/or
the VH region and VL region of the second CAR comprise the amino acid sequences set forth in SEQ ID NO:197 and SEQ ID NO:198, respectively; and/or
the antigen-binding domain comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.
   412. The combination of any of embodiments 396-411, wherein the transmembrane domain is or comprises a transmembrane domain from CD4, CD28, or CD8, optionally from human CD4, human CD38 or human CD8.
   413. The combination of any of embodiments 396-412, wherein:
the transmembrane domain is or comprises a transmembrane domain from a human CD28; and/or
the transmembrane domain is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.
   414. The combination of embodiment 413, wherein the transmembrane domain is or comprises the sequence set forth in SEQ ID NO:18.
   415. The combination of any of embodiments 396-414, wherein the intracellular signaling region comprises an intracellular signaling domain.
   416. The combination of embodiment 415, wherein the intracellular signaling domain is capable of inducing a primary activation signal in a T cell, is a T cell receptor (TCR) component and/or comprises an immunoreceptor tyrosine-based activation motif (ITAM).
   417. The combination of embodiment 415 or embodiment 416, wherein the intracellular signaling domain is or comprises a cytoplasmic signaling domain of a CD3-zeta (CD3ζ) chain or a functional variant or signaling portion thereof, optionally a human CD3 zeta chain.
   418. The combination of any of embodiments 415-417, wherein the intracellular signaling region comprises the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.
   419. The combination of any of embodiments 415-418, wherein the intracellular signaling region further comprises a costimulatory signaling region.
   420. The combination of embodiment 419, wherein the costimulatory signaling region comprises an intracellular signaling domain of a T cell costimulatory molecule or a signaling portion thereof.
   421. The combination of embodiment 419 or embodiment 420, wherein the costimulatory signaling region comprises an intracellular signaling domain of CD28, 4-1BB, or ICOS, or a signaling portion thereof, optionally human CD28, human 4-1BB, or human ICOS.
   422. The combination of any of embodiments 419-421, wherein the costimulatory signaling region comprises:
an intracellular signaling domain of human CD28; and/or
the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.
   423. The combination of any of embodiments 419-422, wherein the costimulatory signaling region comprises:
an intracellular signaling domain of human 4-1BB; and/or
the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.
   424. The combination of any of embodiments 389-423, wherein the encoded second chimeric antigen receptor comprises from its N to C terminus in order: the antigen-binding domain, the spacer, the transmembrane domain and the intracellular signaling region.
   425. A kit comprising the combination of any of embodiments 389-424 and instructions for use, optionally wherein the instructions are for administering a dose of the first and second plurality of cells, optionally in accord with the method or use of any of embodiments 326-387.
   426. An article of manufacture comprising the combination of any of embodiments 389-424 or the kit of embodiment 425.
   427. The article of manufacture of embodiment 426 comprising a first container comprising a dose of the plurality of first cells and a second container comprising a dose of the plurality of second cells, optionally wherein the first and second container independently is a vial or bag.
   428. Use of the combination of any of embodiments 389-424 for the treatment of a disease or disorder, optionally wherein the disease or disorder is a cancer.
   429. The use of the combination of any of embodiments 389-424 for the manufacture of a medicament for treatment of a disease or disorder, optionally wherein the disease or disorder is a cancer.
   430. A pharmaceutical composition for treating a disease or disorder, optionally a cancer, containing the combination of any of embodiments 389-424 as an active ingredient.

### IX. Examples

The following examples are included for illustrative purposes only and are not intended to limit the scope of the invention.

### Example 1: Expression of G Protein-Coupled Receptor Class C Group 5 Member D (GPRC5D) in Multiple Myeloma (MM)

D. GPRC5D mRNA Expression

To identify and evaluate potential cell surface targets for immunotherapy of multiple myeloma (MM), mRNA expression across >1,000 different malignant cell lines, including 30 MM cell lines, were evaluated in silico using the Cancer Cell Line Encyclopedia (CCLE; Barretina et al., Nature. (2012) 483(7391): 603-607). The Genotype-Tissue Expression (GTEx) project database (The GTEx Consortium, Nat Genet. (2013) 45(6): 580-585) was also mined to evaluate mRNA expression in primary, non-malignant tissue types in addition to tumor cells.

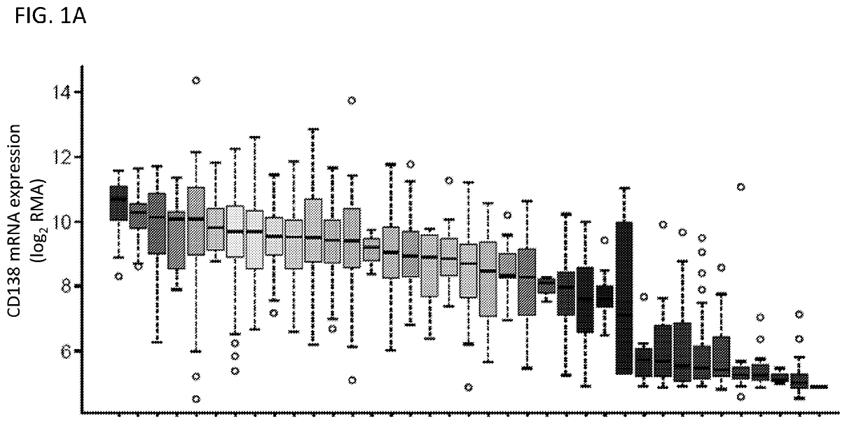

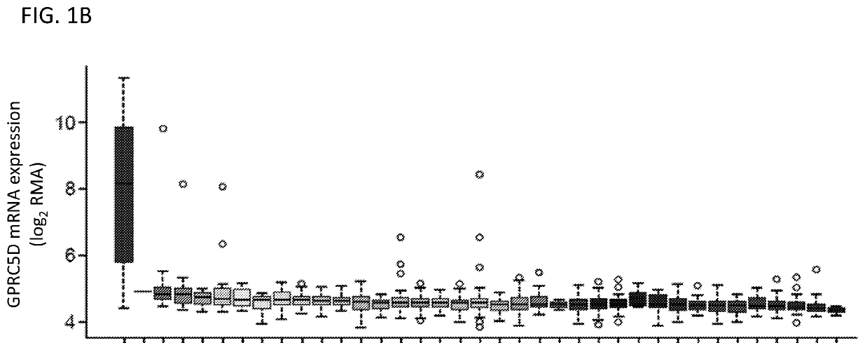

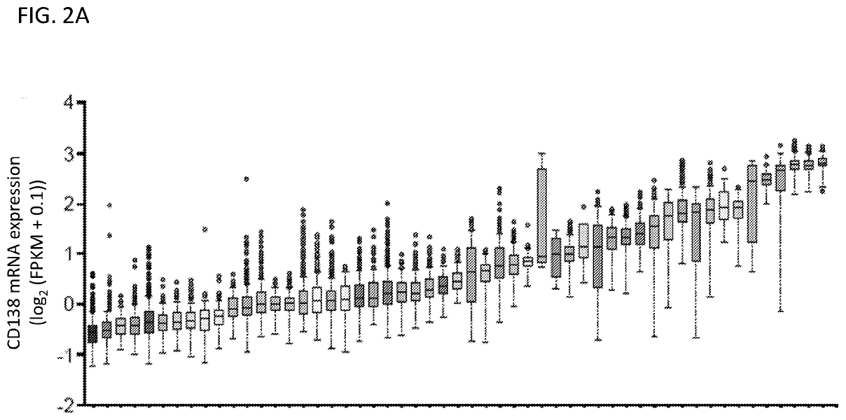

CD138 was used as a control marker, because CD138 is a common surface marker used to identify normal and malignant plasma cells. CD138 was expressed at high levels in MM cell lines and in cell lines from the majority of tumor types, with the highest mean expression in upper aerodigestive track tumors (FIG. 1A). GPRC5D mRNA was highly expressed in MM cell lines (n=30); however, in contrast to CD138, no other tumor type exhibited significant expression (FIG. 1B).

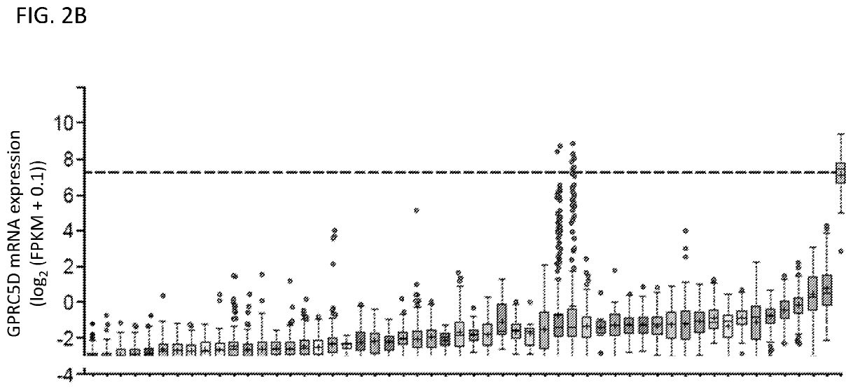

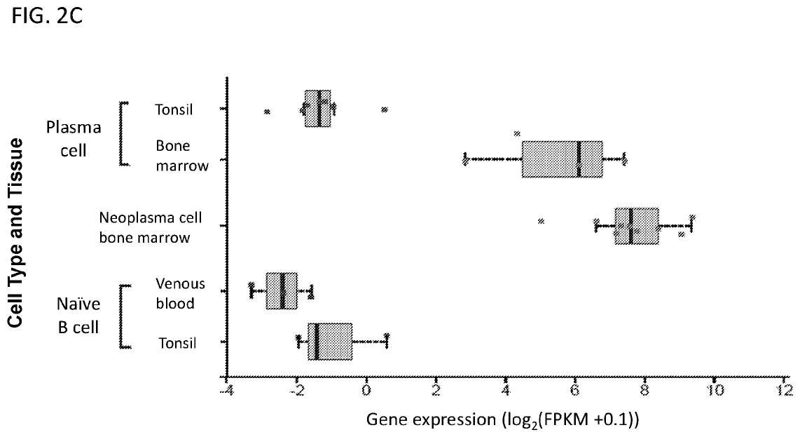

Analysis of expression in primary normal (non-malignant) tissue types from data from the Genotype-Tissue Expression (GTEx) database revealed high CD138 mRNA expression in the esophagus, skin, lung, and liver, among other tissues (FIG. 2A). In contrast, GPRC5D mRNA was not highly expressed in any normal tissues other than variable expression in skin samples (FIG. 2B). Analysis of GTEx mRNA expression in human bone marrow samples showed 1000- and 500-fold higher GPRC5D mRNA expression in primary malignant and normal plasma cells than in B cells from peripheral blood, respectively (FIG. 2C).

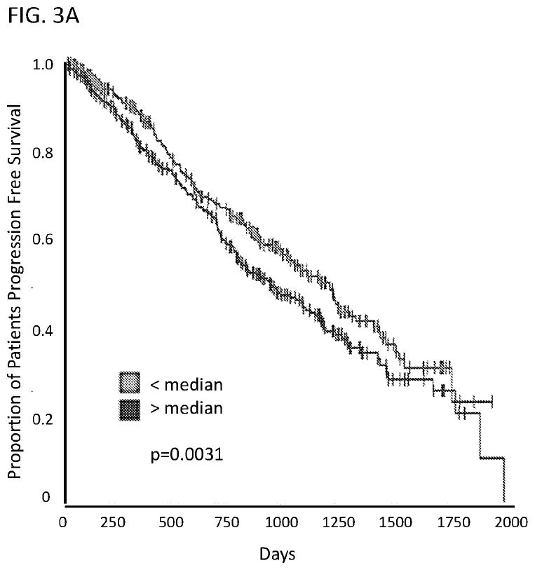

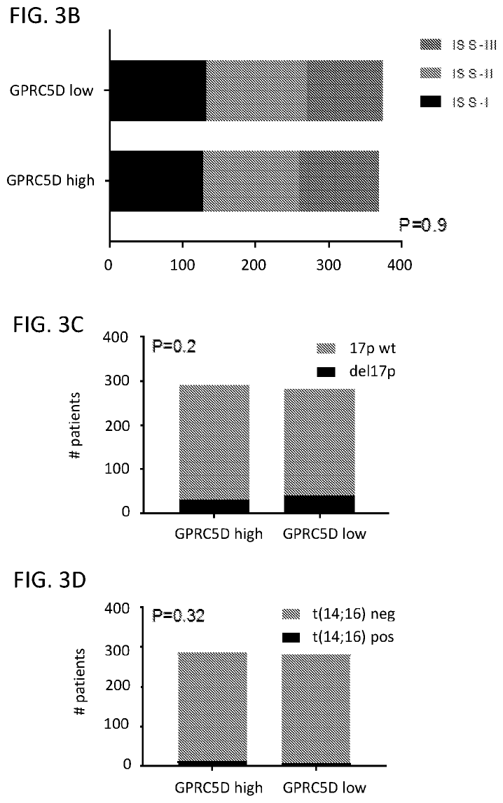

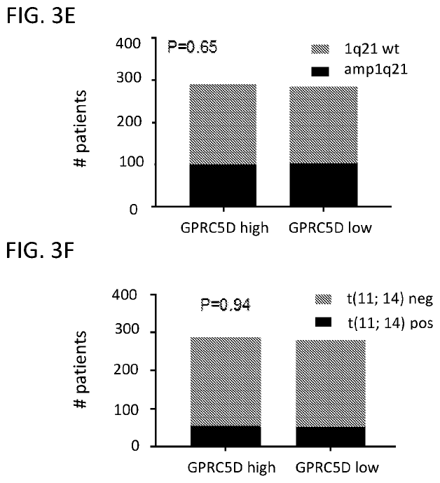

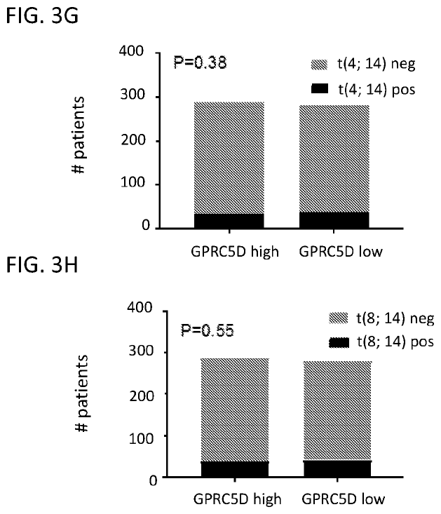

To evaluate the relationship between GPRC5D mRNA expression and clinical outcomes, publicly available CD138-sorted RNAseq expression data (research mmrf.org; version IA13) from 765 patients in the MMRF CoMMpass clinical trial (NCT0145429) was analyzed. Patients were stratified into two groups: (1) those with above median GPRC5D expression and (2) those with below median GPRC5D expression. Higher GPRC5D expression was significantly correlated with shortened progression free survival (FIG. 3A). However, GPRC5D expression was not correlated with international staging system (ISS) score (FIG. 3B; n=369 above median, 374 below median) or any evaluated common cytogenetic abnormalities, including gene amplification, deletion, and translocations (FIG. 3C-3H; n=287-291 above median, 280-282 below median).

E. GPRC5D Protein Expression

1. Immunohistochemistry C) Assay

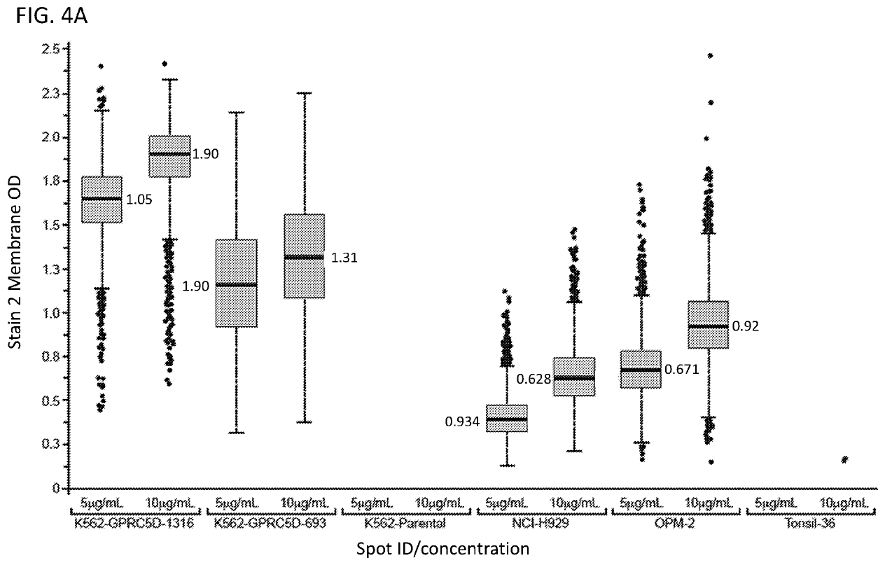

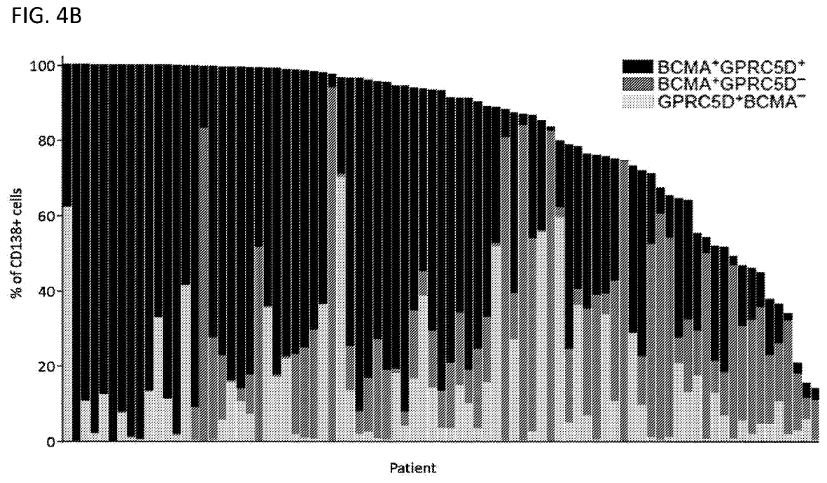

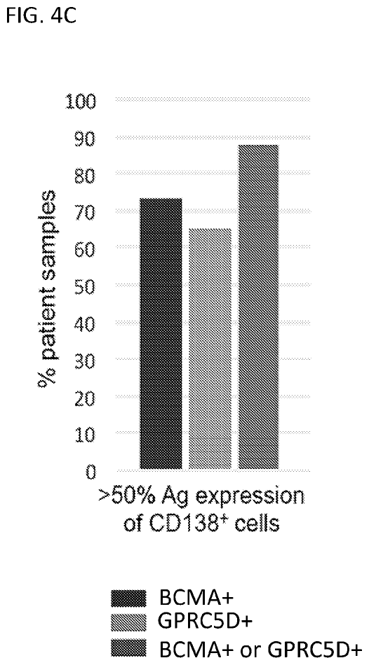

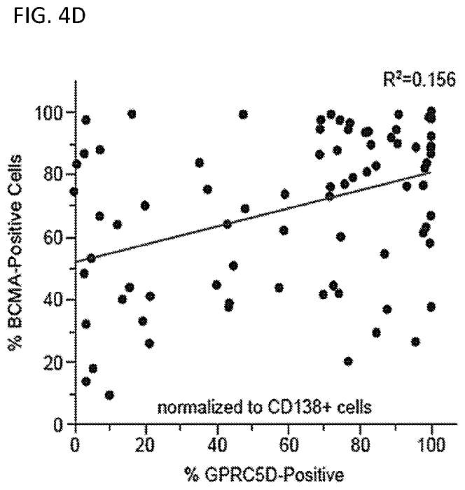

Previous reports evaluating GPRC5D protein in multiple myeloma cells were unable to identify cell surface expression using flow cytometric analyses (Frigyesi. et al. Robust isolation of malignant plasma cells in multiple myeloma. Blood 123, 1336-40 (2014). These negative results were confirmed using various available reagents. Instead, to evaluate GPRC5D protein expression, an anti-GPRC5D immunohistochemistry (IHC) assay was carried out on K562 cells engineered to express GPRC5D (K562-GPRC5D) and human MM cell lines (OPM-2 and NCI-H929), expressing endogenous GPRC5D, using a monoclonal human anti-GPRC5D antibody (Abcam, catalog no. ab55044). Slides were treated with a multimeric horseradish peroxidase-linker antibody-conjugate system and chromogen 3,3′-Diaminobenzidine (seen as brown staining to visualize GPRC5D immunoreactivity) and counterstained with hematoxylin. The GPRC5D-expressing cells demonstrated positive staining, no staining was observed on parental K562 cells, and staining of only rare plasma cells was observed in tissue from primary human tonsil. Outlier boxplot quantification by digital image analysis (Halo) of membrane optical density (mean±interquartile range) on IHC images of each cell line was used to determine the antibody concentration showing the greatest range (FIG. 4A).

2 GPRC5D and BCMA Expression in Multiple Myeloma

CD138, BCMA, and GPRC5D expression were quantified using multiplex quantitative immunofluorescence (Q-IF) on primary bone marrow samples from 83 myeloma patients. Formalin-fixed, paraffin-embedded sections of 83 bone marrow samples from patients with multiple myeloma underwent antigen retrieval before analysis by multiplex immunofluorescence using: (1) rabbit anti-human CD138 and fluorophore rhodamine 6G, (2) mouse anti-human B-cell maturation antigen (BCMA) and fluorophore DCC, and (3) mouse anti-human G protein-coupled receptor family C, group 5, member D (GPRC5D) and fluorophore CY5. Whole sections of stained bone marrow samples were scanned using a Pannoramic P250 slide scanner at set exposure (3DHISTECH, Perkin Elmer, Waltham, Mass.). The percentage of positive cells was based on setting a threshold of positivity for each marker using Halo 2.0 image analysis platform (Indica Labs, Corrales, N. Mex.). Positive and negative cells were counted. Positive cells were selected among CD138+ tumor cells and percentage positive was normalized to CD138+ tumor cells. Prevalence of BCMA and GPRC5D was measured among CD138+ tumor cells with a positivity of 1% or more.

As shown in FIG. 4B, 98% of the bone marrow samples assessed exhibited GPRC5D− positive staining in at least a subset of CD138+ cells. Across samples, most CD138+ cells were positive for both BCMA and GPRC5D, but there were several samples where the predominant population of CD138+ cells expressed only one of the two antigens and not both. The percentage of samples wherein greater than 50% of CD138+ cells were also positive for BCMA, GPRC5D, or BCMA or GPRC5D was also assessed among the 83 primary patient samples (FIG. 4C). Specifically, using a cut-off of greater than or equal to fifty percent antigen expression on CD138+ cells, which has been used in some trials of BCMA-targeted CAR T cell therapy (NCT02215967, NCT02658929), it was observed that 65% (54/83) of samples had GPRC5D expression above this level, 73% (61/83) of samples met this threshold for BCMA, while 88% (73/83) met this threshold when expression of BCMA or GPRC5D was considered (FIG. 4C).

Pearson correlation analysis was used to evaluate the significance of the correlation between BCMA and GPRC5D expression with R² showing fit to the regression line. Analysis indicated GPRC5D expression on CD138+ cells was independent of BCMA expression (R²=0.156; FIG. 4D).

3. GPRC5D Expression in Non-Malignant Tissues

GPRC5D protein expression was evaluated on normal tissue. Core biopsies from 30 different primary normal tissues, each from three human donors, were evaluated by immunostaining. Of these, 24 did not express GPRC5D protein (adrenal, bone marrow, breast (n=5), brain (cerebellum), brain (cerebrum), brain (pituitary), esophagus, heart, liver, lung (no peribronchial glands), mesothelial cell, ovary, peripheral nerve, placenta, prostate, salivary gland, spleen, skeletal muscle, testis, thymus, thyroid, tonsil, uterus, and cervix tissues).

For tissue types that showed any sign of positive staining on IHC, the staining was repeated using samples from non-human primates (cynomolgus monkey; 96% amino acid homology to human; antibody cross-reactive), which yielded similar results. IHC expression results were confirmed by RNA in situ hybridization (RNA-ISH) and, in some cases qPCR, on human, cynomolgus, and murine tissues (Table E1). Among non-plasma cell normal tissues, IHC was positive in cells including from the hair follicle bulb and the peri-bronchial glands, with the hair follicle bulb as the only tissue in which expression was confirmed by RNA in situ hybridization (ISH) and quantitative PCR. Quantitative PCR of skin was weakly positive (Table E1), consistent with expression limited to a rare cell type in the skin.

TABLE E1
GPRC5D expression in non-malignant tissues

| | | Human | | | Cynomolgus Monkey | | Mouse | |
|---|---|---|---|---|---|---|---|---|
| Tissue | Cell Types | IHC | RNA-ISH | qPCR | IHC | RNA-ISH | IHC | RNA-ISH |
| Skin | Hair shafts | High | High | Very | High | High | — | High |
| | | | | weak | | | | |
| | Apocrine | Moderate- | Negative | Very | Low- | Negative | — | NP |
| | glands | High | | weak | Moderate | | | |
| Bronchus | Peribronchial | Low- | Negative | Negative | — | — | — | — |
| | glands | High | | | | | | |
| Lung | Lung | Negative | Negative | — | Negative | Negative | — | Negative |
| | parenchyma | | | | | | | |
| Duodenum | Intestinal | Low- | Negative | Negative | — | — | — | — |
| | epithelium; | Moderate | | | | | | |
| | immune | | | | | | | |
| | cells | | | | | | | |
| Tongue | Mast cells | — | — | — | High | Negative | — | — |
| | and | | | | | | | |
| | eosinophils | | | | | | | |
| Nail bed | glands | — | — | — | Moderate- | Negative | — | — |
| | | | | | High | | | |

— Not Tested
NP: not present in tissue

### Example 2: Generation and Epitope Mapping of Anti-GPRC5D scFvs

A. Generation of anti-GPRC5D scFvs

A human B cell-derived scFv phage display library was used to identify GPRC5D specific scFvs that bound cells expressing GPRC5D, but not cells expressing a non-GPRC5D protein. To generate cells expressing GPRC5D for screening, NIH-3T3 fibroblasts were stably transduced with human GPRC5D cDNA via a retrovirus to generate stable artificial antigen presenting cells (hGPRC5D-aAPCs) for phage display library panning Expression of GPRC5D on the hGPRC5D-aAPCs was confirmed by flow cytometry, and a high expressing subclone was expanded. Phage display was carried out by panning of a human B cell-derived scFv phage display library against the hGPRC5D-aAPCs.

After phage library screening, positive clones were sequenced, and unique clones underwent a second validation step of binding to human MM cell lines MM.1S and NCI-H929, but not to the Acute Myeloid Leukemia (AML) cell line SET2. Thirty-two (32) unique clones were identified with light and heavy chain CDRs covering 5 and 3 subfamilies, respectively, and with HCDR3 lengths ranging from 6-23 amino acids. Seven clones that exhibited the highest specific binding to MM.1S and NCI-H929 cells, but not GPRC5D-negative cell lines (derived from other hematologic malignancies), were selected for development into CAR constructs as described below.

B. Epitope Mapping

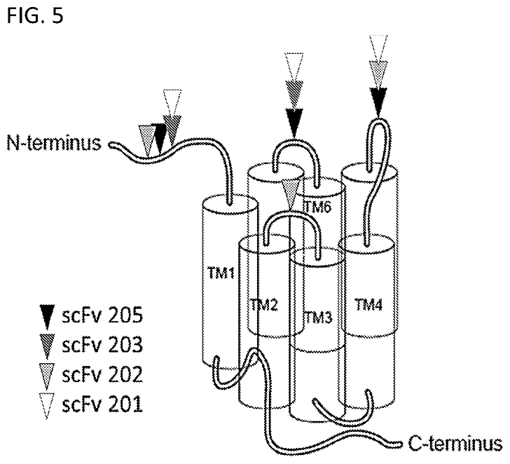

A library of overlapping 15-mer peptides covering the extracellular domains of GPRC5D was synthesized and chemically linked to flexible scaffolds to assess linear, conformational, and discontinuous (via chemical linkage of peptides onto scaffolds) epitope binding of a subset of the GPRC5D targeted scFvs generated above, using ELISA-based methods. The scFvs bound to diverse epitopes, with all extracellular loops of the GPRC5D seven-transmembrane protein bound by at least one of the identified scFvs (FIG. 5).

### Example 3: Generation of Anti-GPRC5D Chimeric Antigen Receptors (CARs) and Cells Expressing Anti-GPRC5D CARs

Chimeric antigen receptors (CARs) were engineered, incorporating GPRC5D-targeted human scFv from 7 candidates identified above in different structural formats (Table E3).

Polynucleotide constructs encoding a CAR were generated that encoded an antigen-binding domain containing each scFv described above, in which the variable heavy chain (V_{H}) and variable light chain (V_{L}) were connected by a linker (containing residues set forth in SEQ ID NO:52) and in which each scFv was generated to be in the V_{H}/V_{L} orientation and V_{L}/V_{H} orientation.

Each generated CAR construct contained the scFv antigen-binding domain; one of three different length immunoglobulin-derived spacer domains [short (hinge only, 12aa; SEQ ID NO: 15); medium (hinge-CH3, 119aa; SEQ ID NO: 16); or long (hinge-CH2-CH3, 228aa; SEQ ID NO: 17, encoded by the sequence set forth in SEQ ID NO:73) with CH2 modifications to limit Fc receptor binding]; a human CD28-derived transmembrane domain (SEQ ID NO: 18); a human 4-1BB-derived intracellular signaling domain (SEQ ID NO: 19); and a human CD3zeta-derived intracellular signaling domain (SEQ ID NO: 20).

Table E3 sets forth the components, and SEQ ID NO, of the exemplary generated anti-GPRC5D CAR constructs.

TABLE E3
Components of GPRC5D CAR constructs

| | SEQ ID NO: | | | | | | | |
|---|---|---|---|---|---|---|---|---|
| | | | | | | | Intracellular | |
| | scFv | | | | | | region | |
| CAR Construct (scFv - spacer) | aa | nt | V_{H} | V_{L} | spacer | CD28 TM | 4-1BB | CD3ζ |
| GPRC5D-200 V_{H}/V_{L} - short | 1 | | 21 | 22 | 15 | 18 | 19 | 20 |
| GPRC5D-200 V_{L}/V_{H} - short | 2 | | 21 | 22 | 15 | 18 | 19 | 20 |
| GPRC5D-201 V_{H}/V_{L} - short | 3 | | 23 | 24 | 15 | 18 | 19 | 20 |
| GPRC5D-201 V_{L}/V_{H} - short | 4 | | 23 | 24 | 15 | 18 | 19 | 20 |
| GPRC5D-202 V_{H}/V_{L} - short | 5 | | 25 | 26 | 15 | 18 | 19 | 20 |
| GPRC5D-202 V_{L}/V_{H} - short | 6 | | 25 | 26 | 15 | 18 | 19 | 20 |
| GPRC5D-203 V_{H}/V_{L} - short | 7 | | 27 | 28 | 15 | 18 | 19 | 20 |
| GPRC5D-203 V_{L}/V_{H} - short | 8 | | 27 | 28 | 15 | 18 | 19 | 20 |
| GPRC5D-204 V_{H}/V_{L} - short | 9 | | 29 | 30 | 15 | 18 | 19 | 20 |
| GPRC5D-204 V_{L}/V_{H} - short | 10 | | 29 | 30 | 15 | 18 | 19 | 20 |
| GPRC5D-205 V_{H}/V_{L} - short | 11 | | 31 | 32 | 15 | 18 | 19 | 20 |
| GPRC5D-205 V_{L}/V_{H} - short | 12 | | 31 | 32 | 15 | 18 | 19 | 20 |
| GPRC5D-206 V_{H}/V_{L} - short | 13 | | 33 | 34 | 15 | 18 | 19 | 20 |
| GPRC5D-206 V_{L}/V_{H} - short | 14 | | 33 | 34 | 15 | 18 | 19 | 20 |
| GPRC5D-200 V_{H}/V_{L} - medium | 1 | | 21 | 22 | 16 | 18 | 19 | 20 |
| GPRC5D-200 V_{L}/V_{H} - medium | 2 | | 21 | 22 | 16 | 18 | 19 | 20 |
| GPRC5D-201 V_{H}/V_{L} - medium | 3 | | 23 | 24 | 16 | 18 | 19 | 20 |
| GPRC5D-201 V_{L}/V_{H} - medium | 4 | | 23 | 24 | 16 | 18 | 19 | 20 |
| GPRC5D-202 V_{H}/V_{L} - medium | 5 | | 25 | 26 | 16 | 18 | 19 | 20 |
| GPRC5D-202 V_{L}/V_{H} - medium | 6 | | 25 | 26 | 16 | 18 | 19 | 20 |
| GPRC5D-203 V_{H}/V_{L} - medium | 7 | | 27 | 28 | 16 | 18 | 19 | 20 |
| GPRC5D-203 V_{L}/V_{H} - medium | 8 | | 27 | 28 | 16 | 18 | 19 | 20 |
| GPRC5D-204 V_{H}/V_{L} - medium | 9 | | 29 | 30 | 16 | 18 | 19 | 20 |
| GPRC5D-204 V_{L}/V_{H} - medium | 10 | | 29 | 30 | 16 | 18 | 19 | 20 |
| GPRC5D-205 V_{H}/V_{L} - medium | 11 | | 31 | 32 | 16 | 18 | 19 | 20 |
| GPRC5D-205 V_{L}/V_{H} - medium | 12 | | 31 | 32 | 16 | 18 | 19 | 20 |
| GPRC5D-206 V_{H}/V_{L} - medium | 13 | | 33 | 34 | 16 | 18 | 19 | 20 |
| GPRC5D-206 V_{L}/V_{H} - medium | 14 | | 33 | 34 | 16 | 18 | 19 | 20 |
| GPRC5D-200 V_{H}/V_{L} - long | 1 | 257 | 21 | 22 | 17 | 18 | 19 | 20 |
| GPRC5D-200 V_{L}/V_{H} - long | 2 | 258 | 21 | 22 | 17 | 18 | 19 | 20 |
| GPRC5D-201 V_{H}/V_{L} - long | 3 | 259 | 23 | 24 | 17 | 18 | 19 | 20 |
| GPRC5D-201 V_{L}/V_{H} - long | 4 | 260 | 23 | 24 | 17 | 18 | 19 | 20 |
| GPRC5D-202 V_{H}/V_{L} - long | 5 | 261 | 25 | 26 | 17 | 18 | 19 | 20 |
| GPRC5D-202 V_{L}/V_{H} - long | 6 | 262 | 25 | 26 | 17 | 18 | 19 | 20 |
| GPRC5D-203 V_{H}/V_{L} - long | 7 | 263 | 27 | 28 | 17 | 18 | 19 | 20 |
| GPRC5D-203 V_{L}/V_{H} - long | 8 | 264 | 27 | 28 | 17 | 18 | 19 | 20 |
| GPRC5D-204 V_{H}/V_{L} - long | 9 | 265 | 29 | 30 | 17 | 18 | 19 | 20 |
| GPRC5D-204 V_{L}/V_{H} - long | 10 | 266 | 29 | 30 | 17 | 18 | 19 | 20 |
| GPRC5D-205 V_{H}/V_{L} - long | 11 | 267 | 31 | 32 | 17 | 18 | 19 | 20 |
| GPRC5D-205 V_{L}/V_{H} - long | 12 | 268 | 31 | 32 | 17 | 18 | 19 | 20 |
| GPRC5D-206 V_{H}/V_{L} - long | 13 | 269 | 33 | 34 | 17 | 18 | 19 | 20 |
| GPRC5D-206 V_{L}/V_{H} - long | 14 | 270 | 33 | 34 | 17 | 18 | 19 | 20 |

Nucleic acid constructs encoding the CARs were generated as bicistronic constructs further encoding an exemplary reporter green fluorescent protein (GFP), which was separated from the CAR sequence by a self-cleaving T2A sequence (sequence set forth in SEQ ID NO:44, encoding the amino acid sequence set forth in SEQ ID NO: 37).

The nucleic acid constructs were cloned into either a lentiviral expression vector or a retroviral expression vector for transduction of cells. For lentiviral constructs, the nucleotide sequence encoding the anti-GPRC5D CARs containing the long spacer were codon optimized and assessed for potential splice sites and modified in a conservative manner, including removal of potential predicted splice sites. Following codon-optimization for expression in human cells, the DNA sequence was then analyzed for splice sites (e.g. NNSPLICE version 0.9 online splice-site prediction tool; fruitfly.org, Berkeley Drosophila Genome Project, Berkeley, Calif.). Splice donor sites and splice acceptor sites were evaluated independently. Identified splice donor and splice acceptor sites with a splice site score of >0.7 (>70% probability of a splice event), e.g., in promoter region and long spacer region), were modified by silent mutation to reduce the splice site score to less than 0.7. Among such regions further modified after codon optimization for splice site elimination were those within longer spacer region sequences. The nucleotide sequences of the modified nucleotide sequence encoding the long spacer of the CAR is set forth in SEQ ID NO: 74.

For transduction of primary human T cells, as described in studies below, primary human T cells were isolated from whole blood obtained from healthy donors or the New York Blood Center (New York, N.Y.). T cells were stimulated with phytohemagglutinin (2 mg/mL) or anti-CD3/anti-CD3 magnetic beads at a 1:1 ratio for 24 hours, in the presence of recombinant IL-2, IL-7 and IL-15. T cells were transduced by spinoculation with virus on days 2 to 3 after initiation of the stimulation, expanded and cells were harvested and, in some cases, cryopreserved and thawed before use. Transduction efficiency was determined by flow cytometric analysis on days 4 to 10.

### Example 4: Assessment of Antigen-independent (Tonic) Signaling from Different Anti-GPRC5D Chimeric Antigen Receptors (CARs)

A stable Jurkat T cell reporter line was generated containing a Nur77 knock-in reporter by targeted integration via homology dependent repair (HDR), where the nucleic acid sequences encoding the reporter molecule was inserted at the endogenous Nur77 locus. An exemplary red fluorescent protein (RFP) reporter molecule was targeted via HDR to be inserted into a Jurkat T cell clone E6-1 (ATCC® TIB-152™) in-frame downstream of the endogenous Nr4a1 (Nur77) gene, prior to the stop codon, and after a “self-cleaving” T2A element (sequence set forth in SEQ ID NO:45, encoding the amino acid sequence set forth in SEQ ID NO: 37), to allow for co-expression of RFP as a reporter of Nur77 expression.

The Nur77-RFP Jurkat T cell line was stably transduced with a bicistronic construct containing GFP and one of the 42 different CAR constructs described in Example 3 above. The Nur77-reporter cell line was used to assess T cell activation in CAR-engineered cells as Nur77 is an immediate early gene product in T lymphocytes; transcription is initiated specifically downstream of CD3 zeta signaling, and is not influenced by cytokine or TLR mediated signals. In this model, tonic signaling was indicated by RFP expression in the absence of GPRC5D antigen stimulation.

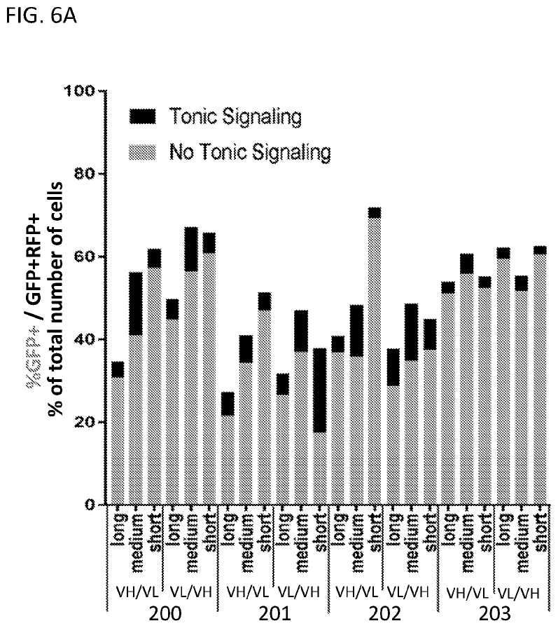

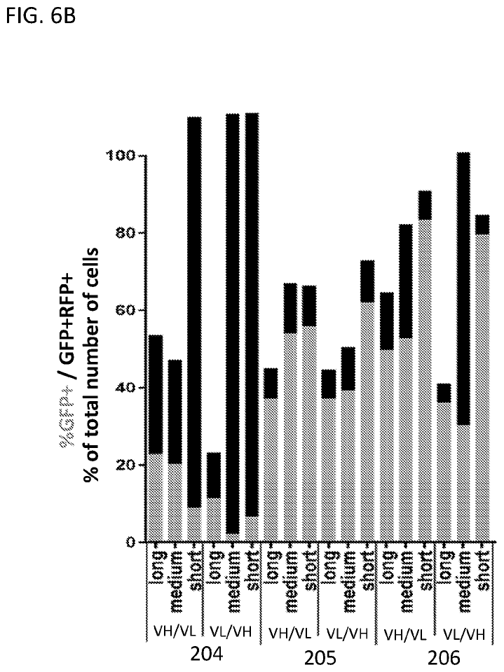

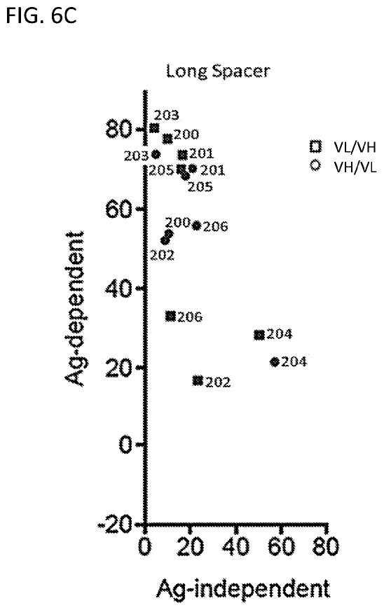

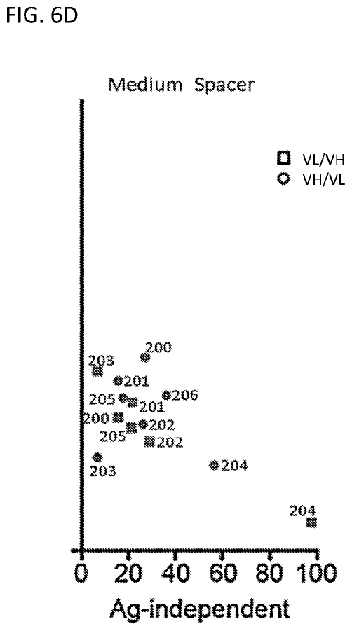

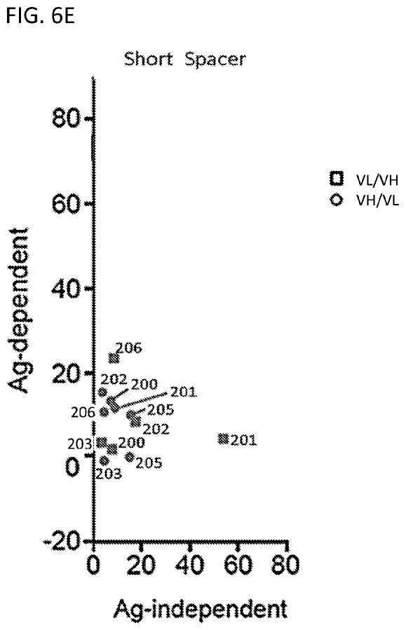

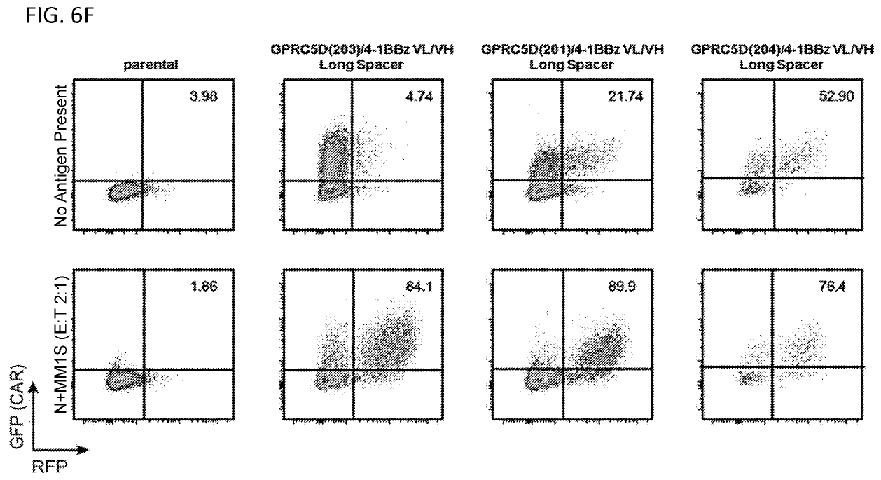

5×10⁵ of each of the CAR-transduced Nur77-RFP Jurkat T cells were incubated in the absence of GPRC5D-expressing cells. Transduction of CAR was measured by GFP signal and tonic signaling was determined as the percent of GFP-expressing (i.e., CAR-transduced) cells that expressed RFP on days 2, 7 and 11 post-transduction. The tonic and non-tonic signaling detected from GFP+ cells is summarized in FIGS. 6A-6B. Results of the assay demonstrated varied tonic signaling among CAR constructs, with the constructs incorporating human GPRC5D-203 scFvs (VL/VH and VH/VL) displaying the lowest tonic signaling. Certain CAR constructs that were associated with the highest levels of tonic signaling in cells expressing such CARs also were observed to be associated with poor growth of the Jurkat reporter cell line and were excluded from further evaluation (GPRC5D-204 VH/VL and VL/VH with the short spacer and GPRC5D-206 VL/VH with medium spacer).

Antigen-independent signaling and antigen-dependent signaling of the CAR constructs were compared. CAR-transduced Nur77-RFP Jurkat T cells or non-transduced parental cells were incubated without the presence of antigen or in the presence of GPRC5D-expressing MM.1S myeloma cells at an effector:target (E:T) ratio of 1:2. GFP and RFP signal was assessed after 20 hours as a measure of transduction of the CAR or as a measure of T cell signaling, respectively. The percent of CAR-transduced Jurkat cells (GFP+) displaying RFP signal in the absence of antigen was plotted against such cells displaying RFP signal in the presence of antigen (FIG. 6C-6E). Results of this assay demonstrated that incorporation of a long spacer increased antigen-mediated signaling through the CAR, but was less likely to induce antigen-independent (tonic) signaling, which was observed with CAR constructs having shorter spacers.

FIG. 6F depicts representative flow plots of exemplary anti-GPRC5D CAR-expressing constructs containing the long spacer, or non-transduced parental cells, following the incubation in the absence (top row of panels; antigen-independent tonic signaling) or presence of GPRC5D antigen (MM.1S myeloma cells) (bottom row of panels, antigen-dependent signaling). CAR-transduced cells are indicated along the y-axis as GFP+, and signaling is indicated along the x-axis as RFP+. Values shown in the upper right quadrant of each panel are the percent of GFP+ cells that were RFP+. Of the tested CARs, GPRC5D-203 VL/VH with the long spacer had the highest percentage activation after antigen exposure, while maintaining the lowest percentage of tonic signaling.

### Example 5: Specificity of Human Anti-GPRC5D CAR

The scFv antigen-binding domain of the GPRC5D-203 V_{L}/V_{H}, long spacer CAR was tested for binding specificity and off-target binding.

A. Cell-Cell Interaction Assay

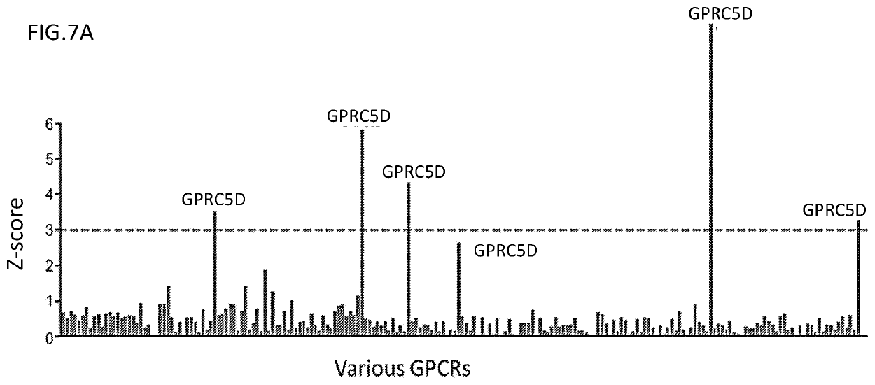

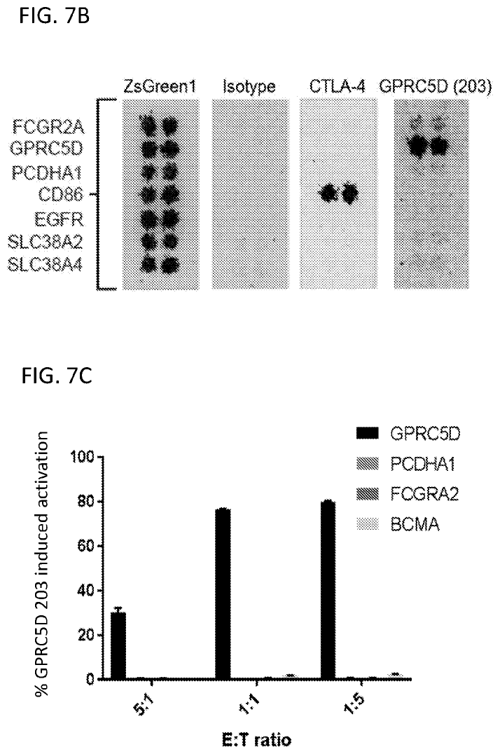

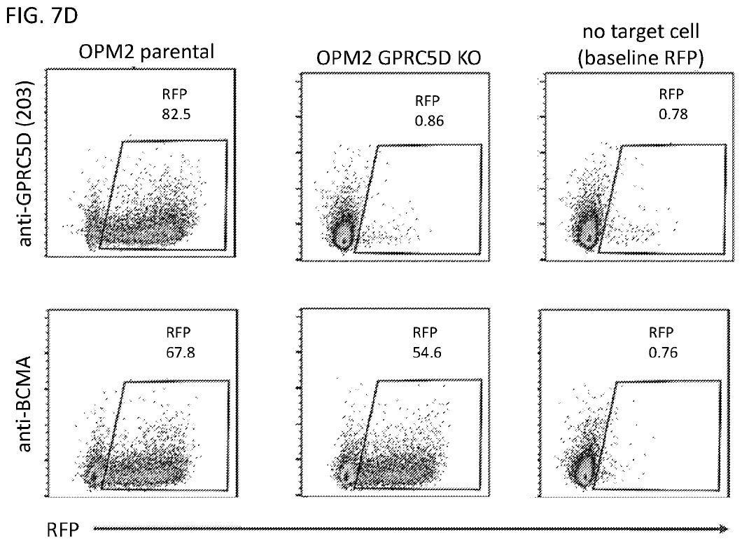

The GPRC5D-203 V_{L}/V_{H}, long spacer CAR but lacking the signaling domains, was tested for binding specificity among G-protein coupled receptors (GPCRs). The non-signaling CAR was transiently expressed in HEK293 cells using a cell surface expression vector that included cytoplasmic mCherry. In parallel, the cDNA of individual receptors from a library of human GPCRs, in a vector expressing cytoplasmic GFP, was transiently expressed in the HEK293 cells. Of these, 202 GPCRs passed quality control of >25% transduction and were screened for off-target binding. Binding was determined using an automated flow cytometric assay that detects cell-cell interaction. The exemplary GPRC5D CAR interacted exclusively with GPRC5D compared to all GPCRs evaluated (FIG. 7A; pre-specified threshold for significance (horizontal line): Z-score 3; P<0.0027).

B. Cell Microarray Assay

The specificity of GPRC5D-203 V_{L}/V_{H}, long spacer CAR was confirmed in an scFv-Fc IHC assay. Individual HEK293 cell populations, each expressing one of 4,417 human plasma membrane proteins, were grown in cell microarray spots on a microscope slide and screened with the anti-GPRC5D-203 V_{L}/V_{H} scFv (SEQ ID NO: 8) fused to an mIgG2a Fc antibody, an mIgG2a Fc isotype negative control. Cell microarrays were assessed for binding by automated fluorescent microscopy with a fluorescently labeled anti-mIgG2a secondary antibody.

Results indicated strong binding to GPRC5D and weak to medium potential off-target binding to two additional proteins, protocadherin alpha 1 (PCDH1A) and Fc gamma receptor 2A (CD32a; FCGR2A), a protein with known potential for Fc-interaction. A small-scale second screen of cells expressing these proteins indicated potential for binding (FIG. 7B).

C. Off-Target Antigen-Dependent Activation

To evaluate the functional activity based on off-target effects, the potential off-target binders, PCDH1A and FCGR2A, were expressed in K562 cells and co-cultured with the Jurkat Nur77/RFP reporter cells transduced to express the GPRC5D-203 V_{L}/V_{H}, long spacer CAR, using the assay described in Example 4. K562 cells expressing BCMA were used as a negative control. The cells were incubated at E:T ratios of 5:1, 1:1 and 1:5. The percent RFP+ cells of the GFP+ CAR-transduced cells was determined for each condition. As shown in FIG. 7C, only co-culture with K562-GPRC5D cells resulted in activation of the anti-GPRC5D CAR-expressing reporter cells; co-culture of K562-PCDH1A, K562-FCGR2A, or K562-BCMA did not activate the anti-GPRC5D CAR-expressing reporter.

D. GPRC5D-Dependent Activation

To further determine whether the GPRC5D 203 V_{L}/V_{H}, long spacer CAR specifically recognizes GPRC5D, GPRC5D was knocked out of OPM2 cells using a CRISPR-Cas9 system. Briefly, five CRISPR RNA (crRNA) guides (gRNA) were chosen proximal to and within GPRC5D exon 1 (SEQ ID NO: 292-296). crRNAs were complexed with Alt-R CRISP-Cas9 trans-activating crRNA (tracrRNA) at a 1:1 ratio to generate two-part gRNAs. All 5 gRNAs were pooled and incubated with Cas9 at a 2:1 ratio to generate ribonucleoprotein (RNP) complexes. OPM2 cells were electroporated with Cas9 RNP targeting GPRC5D. Electroporated cells were expanded and cloned, with expanded clones screened by polymerase chain reaction (PCR) to confirm deletions at the GPRC5D locus.

OPM2 parental or GPRC5D-knockout cells were cultured at a 1:1 ratio with Jurkat Nur77RFP cells (described in Example 4) that were engineered to express the GPRC5D-203 V_{L}/V_{H}, long spacer CAR or an anti-BCMA CAR, and incubated for 20 hours. Activation of the CAR was assessed by measuring changes in RFP expression by flow cytometry. As shown in FIG. 7D, activation of Jurkat Nur77/RFP reporter cells mediated by the GPRC5D-203 V_{L}/V_{H} CAR after co-culture with OPM2 cells was abolished when they were instead co-cultured with OPM2 cells in which GPRC5D was knocked out using CRISPR-Cas9.

These results revealed that the anti-GPRC5D-203 (V_{L}/V_{H}), long spacer CAR specifically recognizes GPRC5D.

### Example 6: In Vitro Activity of GPRC5D Targeted CAR T Cell Therapy

CAR T cells incorporating the GPRC5D-203 V_{L}/V_{H}, long spacer CAR were tested for functional activity based on cytotoxic activity and ability to induce production of cytokines in the presence of antigen. To generate anti-GPRC5D CAR-expressing T cells, T cells were isolated by immunoaffinity-based enrichment from leukapheresis samples from human donor subjects. Isolated T cells were activated and transduced with retroviral vectors containing the polynucleotide construct encoding the anti-GPRC5D-203 VL/VH, long spacer CAR, as described in Example 3. As controls, T cells from the same donor also were transduced with a CAR incorporating an anti-BCMA scFv or an irrelevantly targeted anti-CD19 scFv, or were mock transduced.

A. Cytoxicity

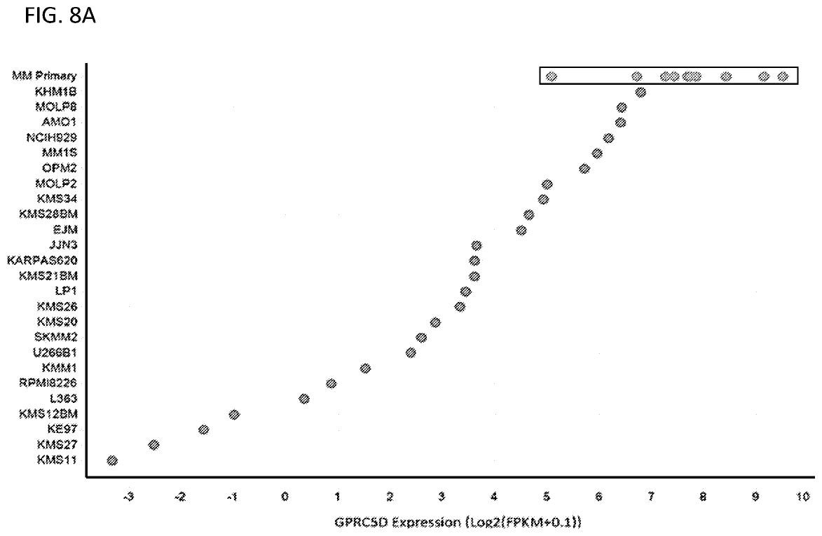

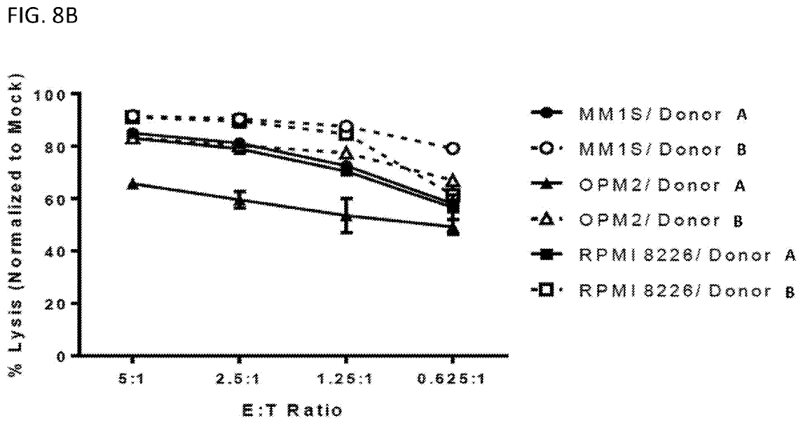

A panel of multiple myeloma cell lines and primary multiple myeloma cells were evaluated for GPRC5D mRNA expression by CCLE RNAseq for MM cell lines or Blueprint RNAseq for primary MM cells (FIG. 8A; primary multiple myeloma cells shown in the box). CAR T cells were co-cultured with MM1.S, OPM2, and RPMI-8226 cell lines at a range of effector to target (E:T) ratios for 24 hours, and cell lysis was monitored. As shown in FIG. 8B, anti-GPRC5D CAR-expressing T cells induced cytotoxicity in all three cell lines, normalized to donor-matched, mock-transduced T-cells.

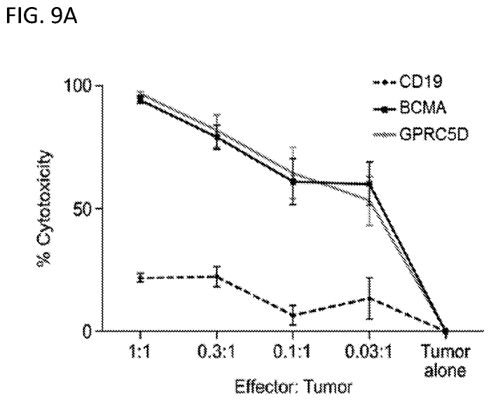

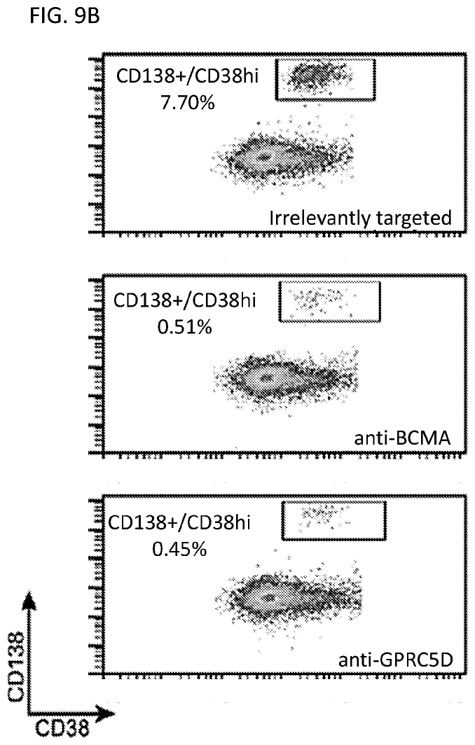

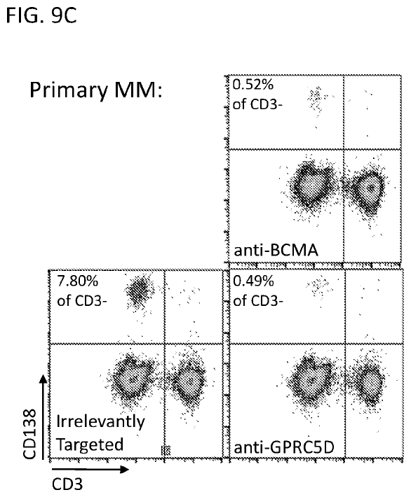

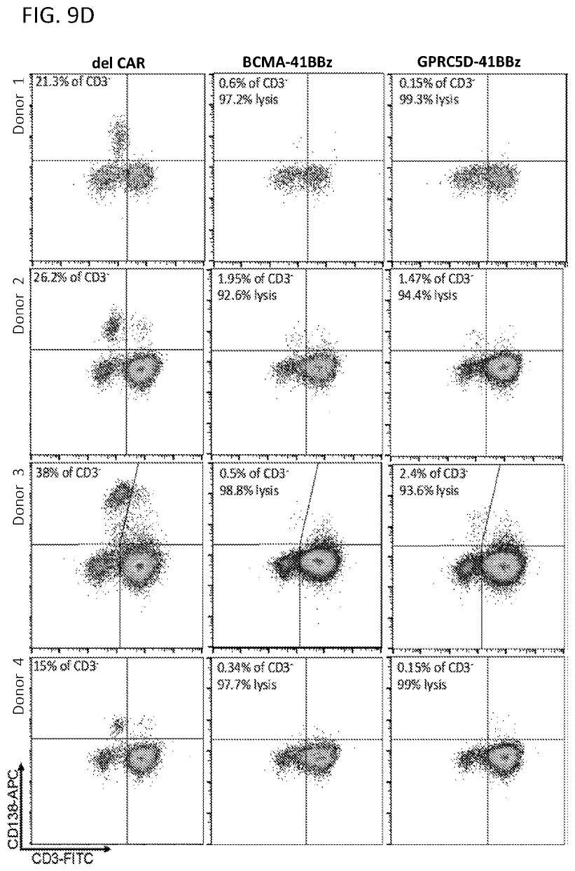

CAR T cells were co-cultured with OPM2 human MM target cell line (which express endogenous BCMA and GPRC5D) engineered to express firefly luciferase (OPM2-ffLuc MM) at a range of E:T ratios for 24 hours Killing of target cells was determined by ATP-dependent bioluminescence after the addition of luciferin, and was normalized to killing in cultures with target tumor cells alone (N=3, mean±stdev). As shown in FIG. 9A, anti-GPRC5D CAR-expressing T cells induced cytotoxicity across a broad range of effector to target (E:T) ratios from 80% at 0.03:1 E:T ratio to 98% at 1:1 E:T ratio. These results were comparable to co-culture with the anti-BCMA CAR T cells, with cytotoxicity from both MM antigen targeted co-cultures significantly above background cytotoxicity seen when OPM2-ffLuc MM cells were co-cultured with irrelevantly targeted anti-CD19 CAR T cells.

Cytotoxicity against primary cells also was assessed by co-culture overnight, e.g. 24-48 hours, of CAR T cells with primary bone marrow mononuclear cells (BMMCs) from the aspirates of patients with multiply relapsed MM at 1:1 CAR+ T cells:BMMCs Killing of primary bone marrow aspirate MM cells from a donor was assessed by flow cytometry based on the percentage of viable BMMCs that were CD138+/CD38hi after gating on CD3 negative cells. Flow cytometric analyses demonstrating killing of primary bone marrow aspirate MM cells (CD138+/CD38hi) are depicted in FIG. 9B. Co-culture of BMMCs cells with either GPRC5D- or BCMA-targeted CAR T cells, but not CD19-targeted (“irrevelantly targeted”) CAR T cells, from the same donor reduced the killing of CD138+/CD38hi fraction by >90% (FIG. 9B; box). To avoid contribution by T cell expansion or transduction efficiency, the percentage of BMMCs that were CD138+/CD3− was determined, as depicted in FIG. 9C. Flow cytometric analyses of BMMCs co-cultured with T cells incorporating either a BCMA-targeted CAR with no signaling domains (“del CAR”) or the indicated BCMA- or GPRC5D-targeted scFvs, from additional donors, are shown in FIG. 9D. In all cases, anti-GPRC5D CAR-T cells induced cytotoxicity significantly above T cells having the CAR with no signaling domain (“del CAR”), and comparably to BCMA-targeted CAR T cells.

B. Cytokine Secretion

CAR T cells were co-cultured 1:1 with OPM2 MM cells, or alone, for 24 hours, and supernatant was collected for analysis of cytokines by Luminex® multiplex assay.

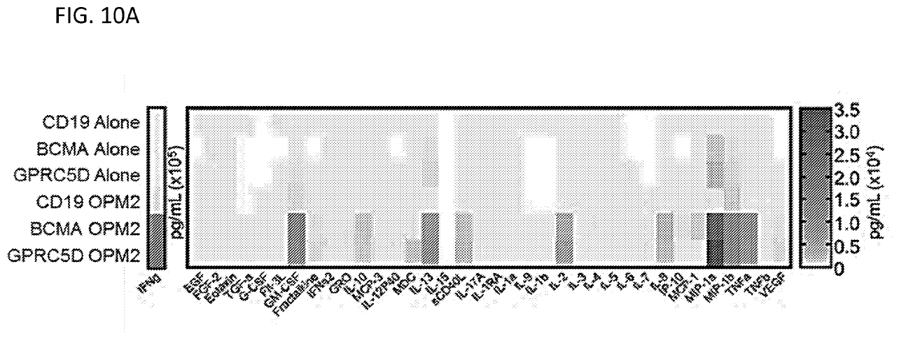

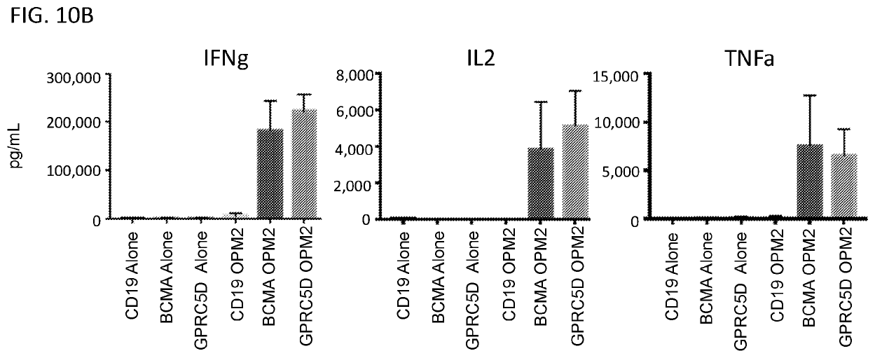

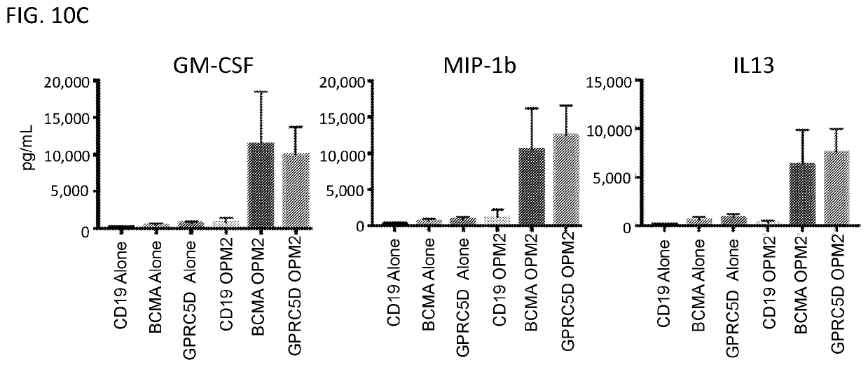

As shown in FIGS. 10A to 10C, cytokine secretion profiles after co-culture with the OPM2 MM cell line were comparable between GPRC5D or BCMA targeted CAR T cells across cytokines evaluated. Notably, CAR T cells targeting either antigen secreted a polyfunctional cytokine profile with the largest increases of IFNγ, MIP-1a, TNFα (effector); GM-CSF, IL2 (stimulatory); MIP-1b (chemo-attractive); sCD40L, and IL13 (regulatory) cytokines when compared to irrelevantly targeted CD19-targeted control CAR T cells co-cultured with OPM2 cells, or CAR T cells cultured in the absence of target cells.

### Example 7: Proliferation and Activation Activity of GPRC5D Targeted CAR T Cell Therapy

CAR T cells incorporating the GPRC5D-203 VL/VH, long spacer CAR were tested for proliferation and activation activity following co-culture with antigen-expressing cells. To generate anti-GPRC5D CAR-expressing T cells, T cells were isolated by immunoaffinity-based enrichment from leukapheresis samples from human donor subjects. Isolated T cells were activated and transduced with lentiviral vectors containing the polynucleotide construct encoding the anti-GPRC5D-203 VL/VH, long spacer CAR, as described in Example 3, except that the construct did not include nucleotides encoding the GFP tag. As controls, T cells from the same donor also were transduced with a CAR incorporating an anti-BCMA scFv, or were mock transduced.

Mock transduced T cells or anti-GPRC5D-203 VL/VH, long spacer CAR T cells were cultured alone (no target), with B-cell acute lymphoblastic leukemia (B-ALL) cells (Nalm6; GPRC5D−), or MM cells (OPM2; GPRC5D+) at a effector (T cell): target (cell line) ratio of 1:1. T cells were stained with CellTrace Violet (CTV) before co-culture, and stained for CD4, CD8, and CD25 after 72 hrs. Proliferation was measured as a dilution of CTV fluorescence in CD4+ and CD8+ T cells, and activation was measured by increased CD25 (Interleukin-2 receptor alpha; IL2RA) fluorescence, indicating upregulation of CD25.

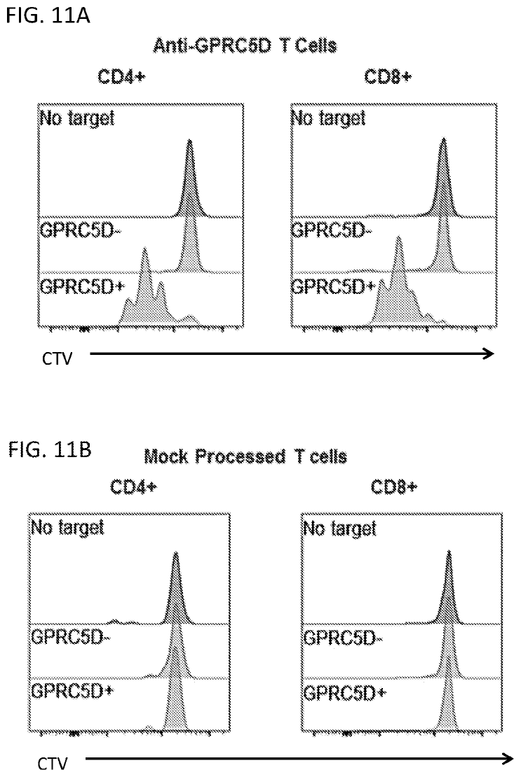

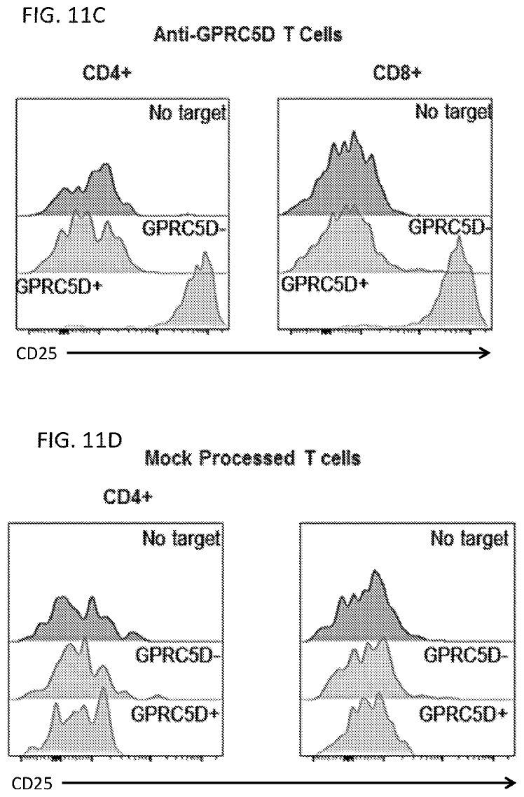

Representative data are set forth in FIGS. 11A-D, which showed that the responses were similarly specific. Anti-GPRC5D CAR T cells proliferated (as indicated by dilution of CellTrace Violet in FIGS. 11A and 11B) and upregulated the activation marker CD25 in the presence of OPM2 cells, but not upon co-culture with B-ALL Nalm6 cells (as shown in FIGS. 11C and 11D). Mock-transduced cells did not respond to MM target cells.

### Example 8: In Vivo Activity of GPRC5D-Targeted CAR T Cell Therapy

An OPM2 human myeloma cell line xenograft model, which leads to bone marrow predominant disease, was used to evaluate the in vivo effects of GPRC5D-targeted CAR T cell therapy. NOD scid gamma (NSG™) mice were injected via tail vein with 2×10⁶ OPM2-ffLuc cells, which were allowed to engraft and expand for 14 or 21 days before a single treatment with a tail vein injection of 3×10⁶ CAR T cells. Control mice did not receive T cells (“tumor only”) or received mock-processed T cells from the same donor (“mock”). Bioluminescent imaging (BLI) of ffLuc was used to monitor tumor burden.

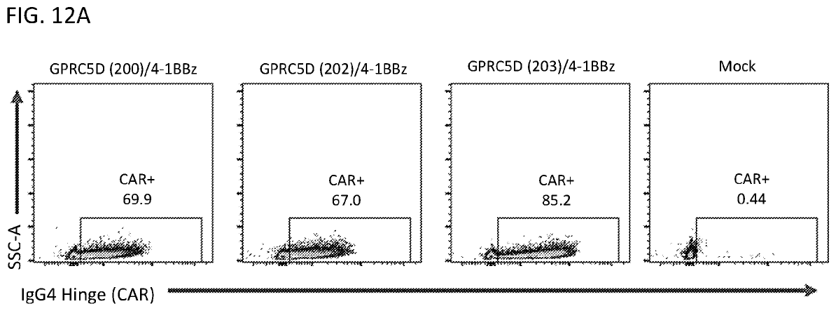

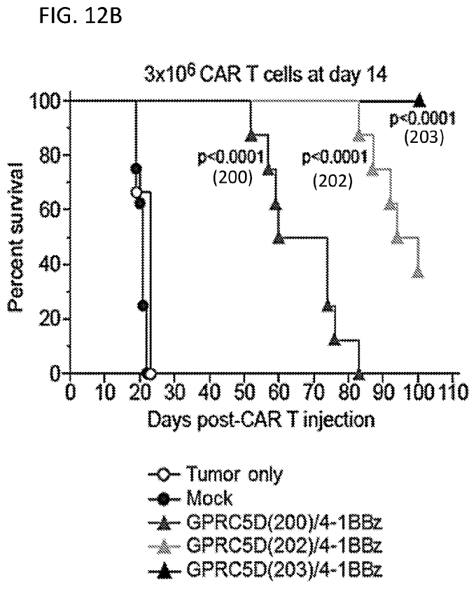

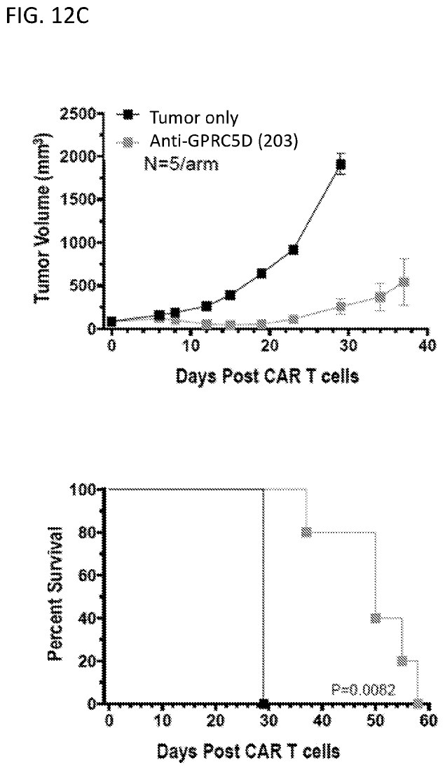

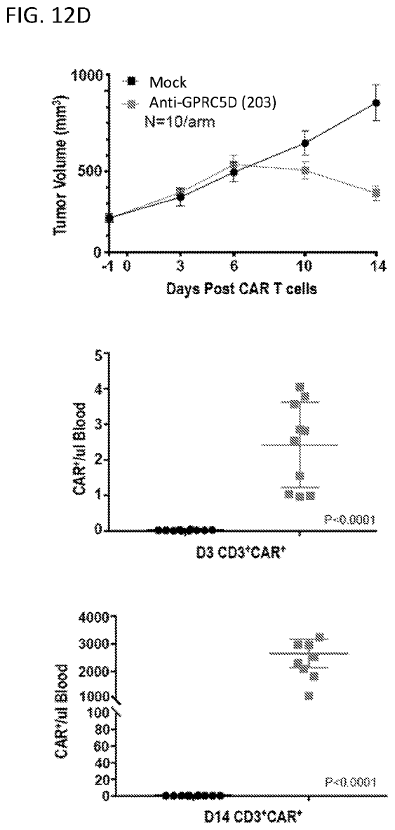

Lentiviral vectors containing polynucleotides encoding anti-GPRC5D CAR as described in Example 3, except without nucleotides encoding the GFP tag, were used to transduce T cells that were isolated by immunoaffinity-based enrichment from leukapheresis samples from human donor subjects. The assessed CARs included one of three anti-GPRC5D scFv, GPRC5D-200 V_{L}/V_{H} scFv (SEQ ID NO: 2), GPRC5D-202 V_{H}/V_{L} scFv (SEQ ID NO: 5), or GPRC5D-203 V_{L}/V_{H} scFv (SEQ ID NO: 8); a long spacer, a human CD28-derived transmembrane domain, a human 4-1BB costimulatory domain and a CD3zeta intracellular signaling domain, as described in Example 3. As shown in FIG. 12A, FACS analysis revealed that the different CAR vectors were expressed comparably on the surface of the T cells, as measured using an antibody specific to the spacer.

Survival of tumor-bearing mice administered 3×10⁶ CAR T cells was monitored 14 days post-OPM2 injection (8 mice per CAR construct). As shown in FIG. 12B, treatment with each of the GPRC5D targeted CAR T cells increased survival of mice in this model compared to no treatment or treatment with mock transfected T cells. At 100 days post-CAR T cell injection, only mice treated with the CAR incorporating GPRC5D-203 V_{L}/V_{H}, long spacer maintained 100% survival.

In addition to the OPM2 model, a GPRC5D low mRNA expressing human multiple myeloma cell line, RPMI-8226, xenograft model was used to evaluate the in vivo effects of GPRC5D-targeted CAR T cell therapy. NSG mice were injected subcutaneously with RPMI-8226 cells. When tumors were palpable (day 0), mice were stratified into treatment and control groups with comparable tumor volumes and treated with a single intravenous injection of 3×10⁶ cells expressing the GPRC5D-203 V_{L}/V_{H}, long spacer. Control mice did not receive T cells (“tumor only”) or received mock-processed T cells (“mock”). Early anti-tumor efficacy was monitored by tumor volume, and CAR T cell expansion was monitored by flow cytometry of peripheral blood at days 3 and 14. As shown in FIGS. 12C and 12D, T cells genetically modified to express a GPRC5D-targeted CAR mediated anti-tumor activity and in vivo CAR T cell expansion was observed.

### Example 9: Effects of Different Co-Stimulatory Domains on In Vivo Activity of GPRC5D-Targeted CAR T Cell Therapy

An OPM2 human myeloma cell line xenograft model, which leads to bone marrow predominant disease, was used to evaluate the in vivo effects of GPRC5D-targeted CAR T cell therapy. NOD scid gamma (NSG™) mice were injected via tail vein with 2×10⁶ OPM2-ffLuc cells which were allowed to engraft and expand for 14 or 21 days before a single treatment with a tail vein injection of 3×10⁶ CAR T cells. Control mice did not receive T cells or received mock-processed T cells from the same donor. Bioluminescent imaging (BLI) of ffLuc was used to monitor tumor burden.

To generate anti-GPRC5D CAR-expressing T cells, polynucleotide constructs encoding CARs as described in Example 3, followed by nucleotides encoding external Gaussian luciferase (extGLuc) for in vivo tracking, were cloned into retroviral vectors, and used to transduce T cells that were isolated by immunoaffinity-based enrichment from leukapheresis samples from human donor subjects. The assessed CAR included GPRC5D-203 V_{L}/V_{H} scFv (SEQ ID NO: 8); a long spacer, a human CD28-derived transmembrane domain, a human 4-1BB costimulatory domain and a CD3zeta intracellular signaling domain, as described in Example 3. A CAR construct incorporating the GPRC5D-203 V_{L}/V_{H} scFv also was generated as described above, except in which the 4-1BB costimulatory signaling region was substituted with a CD28 costimulatory signaling region (SEQ ID NO:46).

Survival and tumor growth were monitored following treatment of OPM2 tumor-bearing mice with 3×10⁶ anti-GPRC5D CAR T cells containing either a 4-1BB costimulatory signaling domain or a CD28-derived costimulatory signaling domain to compare activity of CAR T cells containing different costimulatory signaling regions. In this example, the mice were treated 21 days post-OPM2 injection to provide a larger tumor burden prior to treatment, and 5 mice were treated per CAR construct.

CAR T cells contained a CAR incorporating the GPRC5D-203 V_{L}/V_{H}, long spacer CAR with a 4-1BB-derived costimulatory domain (designated GD-41BBz), or the same CAR but with a CD28-derived co-stimulatory signaling domain (designated GD-CD28z). Treatment with CAR T cells expressing a CAR targeted to an irrelevant protein, CD19, with the long spacer and CD28 co-stimulatory domain (designated 19-CD28z) was also studied as a negative control. In this experiment, the CAR expression vectors used to transduce T cells also included a chimeric exterior Gaussia luciferase (extGLuc) gene with a CD8 transmembrane domain separated from the CAR coding sequence by a P2A element. ExtGLuc expressing cells can be imaged after injection of coelenterazine, a distinct substrate from the luciferin required for ffLuc BLI, which allowed for in vivo BLI of tumor cells (via ffLuc) and CAR T cells (via ExtGLuc) in the same mouse.

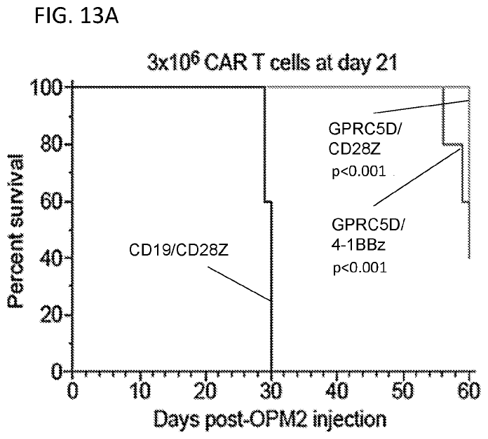

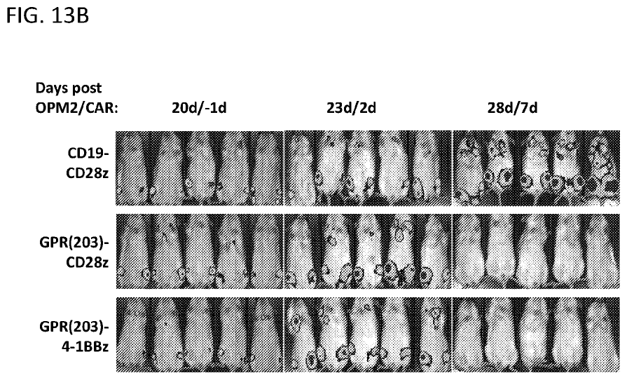

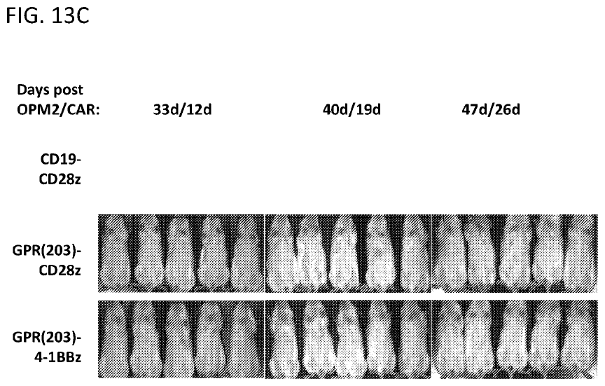

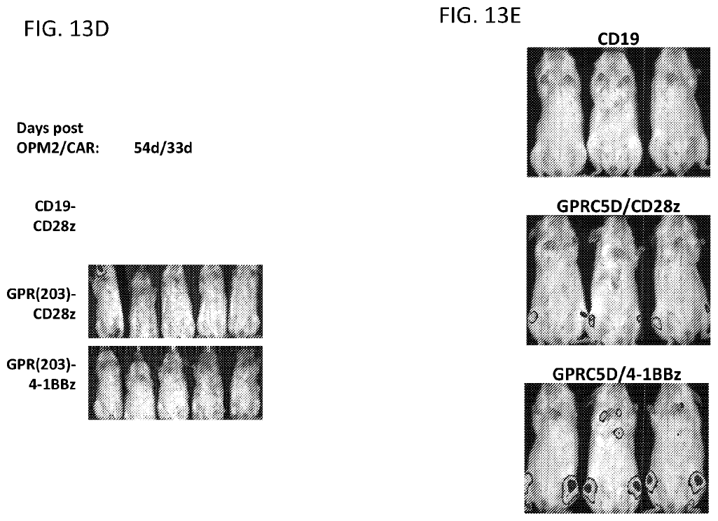

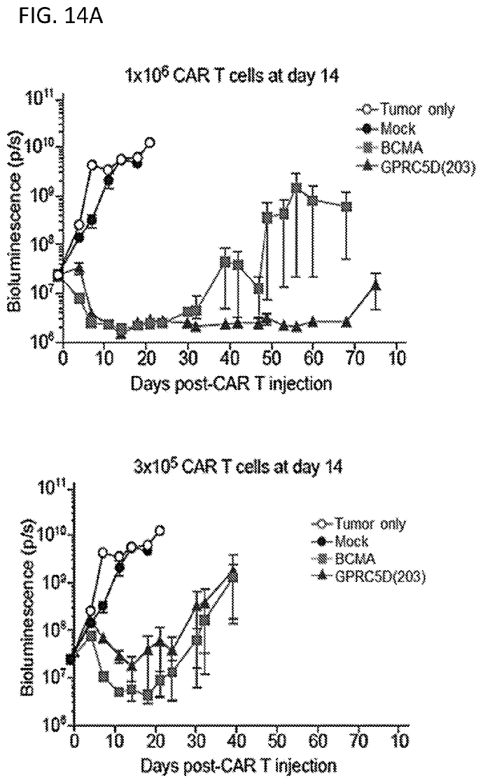

Survival of the treated mice was comparably extended following treatment with either anti-GPRC5D CAR construct regardless of the co-stimulatory signaling domain, compared to mice treated with anti-CD19 CAR T cells (FIG. 13A).

BLI with luciferin of OPM2-ffLuc tumor cells indicated that anti-GPRC5D CAR constructs with either co-stimulatory domain eradicated OPM2 cells between day 2 and 7 after CAR T cell injection, and eradication was predominately durable (FIGS. 13B-D). In contrast, tumor growth continued in mice following treatment with anti-CD19 CAR T cells. The deaths of anti-GPRC5D CAR T cell-treated mice around day 60 in absence of OPM2 BLI signal was secondary to xenogeneic GvHD, a known, donor dependent, limitation of injecting human T cells into NSG mice (King et al. (2009) Clin. Exp. Immunol. 157:104-118; Covassin et al. (20110 Clin. Exp. Immunol. 166:269-280).

The ability of CAR T-cells to home to MM was assessed by monitoring coelenterazine BLI of CAR T-Cells expressing extGLuc 7 days post-CAR T-cell treatment. As shown in FIG. 13E, GPRC5D targeted CAR T cells, with either co-stimulatory domain, localized to the site of the MM xenograft, visualized after coelenterazine injection one week post treatment.

### Example 10: Dose Response of Anti-GPRC5D and Anti-BCMA CAR T Cell Therapy In Vivo in Myeloma Model

In a further study, anti-GPRC5D and anti-BCMA CAR T cell therapies, with an identical CAR backbone, were evaluated in the OPM2 human myeloma cell line xenograft model. The anti-GPRC5D CAR contained GPRC5D-203 V_{L}/V_{H} scFv (SEQ ID NO: 8) with a long spacer, a human CD28-derived transmembrane domain, a human 4-1BB costimulatory domain and a CD3zeta intracellular signaling domain as described in Example 3. The anti-BCMA CAR was generated on the same backbone, except containing an anti-BCMA scFv instead of an anti-GPRC5D scFv. Polynucleotide constructs encoding the CARs were cloned into a lentiviral vector and used to transduce T cells that were isolated by immunoaffinity-based enrichment from leukapheresis samples from a human donor.

About 2×10⁶ OPM2-ffLuc cells were injected into NOD scid gamma (NSG™) to generate an OPM2 human myeloma cell line xenograft model substantially as described in Example 7. Fourteen (14) days post-OPM2 injection, mice were administered a low dose (3.3×10⁵) or higher dose (1×10⁶) of anti-GPRC5D CAR-expressing T cells or anti-BCMA CAR-expressing T cells (N=8/condition). Untreated mice and mice administered mock-transduced T cells were also monitored for comparison (N=8/condition). Survival and tumor burden (BLI of OPM-ffLuc) were monitored every 3 to 9 days for up to 90 days.

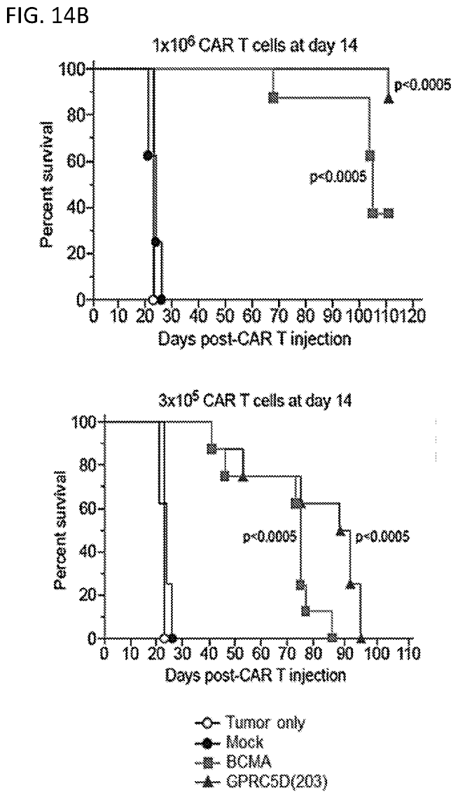

GPRC5D-targeted CAR T cells were comparable to or better than BCMA-targeted CAR T cells in inducing tumor regression (FIG. 14A; triangles vs. squares, respectively), and in extending survival (FIG. 14B; triangles vs. squares) across both doses in treated mice. A dose response was noted in the kinetics of tumor regression for both types of CAR T cell therapy (FIG. 14A).

### Example 11: Evaluation of On Target/Off Tumor Effects

The effects of non-tumor, on-target effects of GPRC5D-targeted CAR T cell therapy was studied in cellular, murine and non-human primate model systems, in part because of GPRC5D expression detected in hair follicle (see Example 1, Table E1).

A. Effect of Essential Normal Cells on GPRC5D-Targeted CAR T Cells

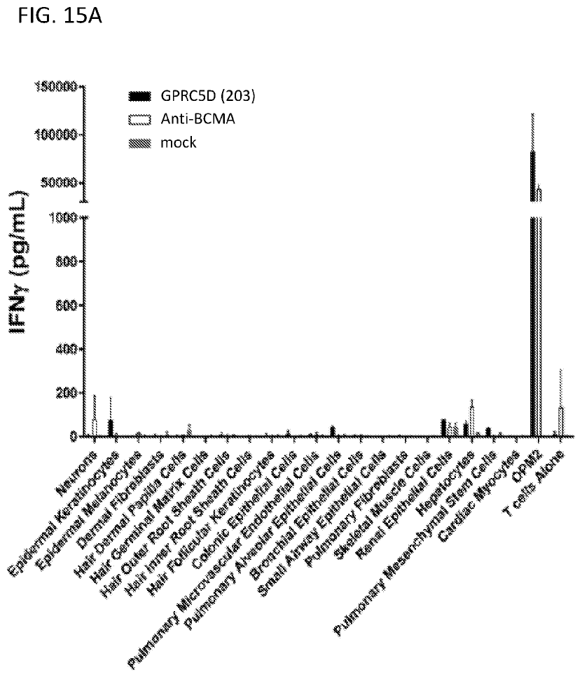

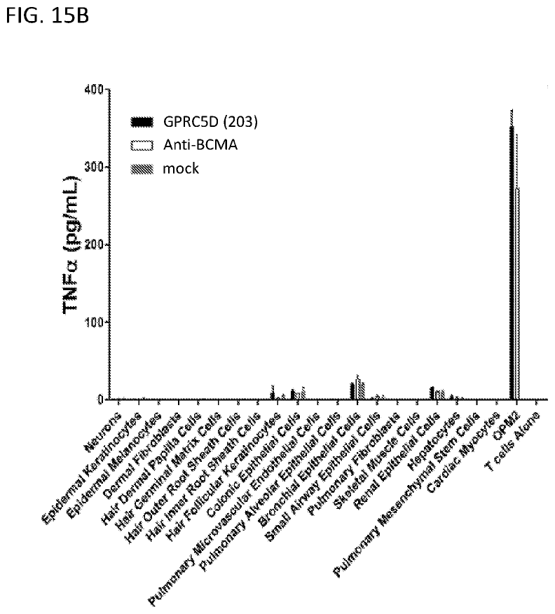

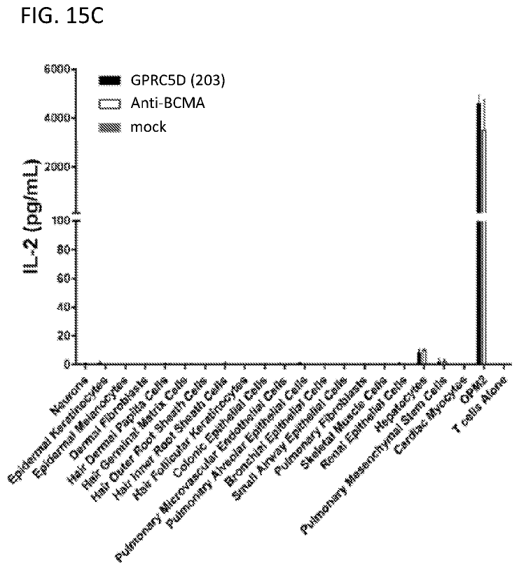

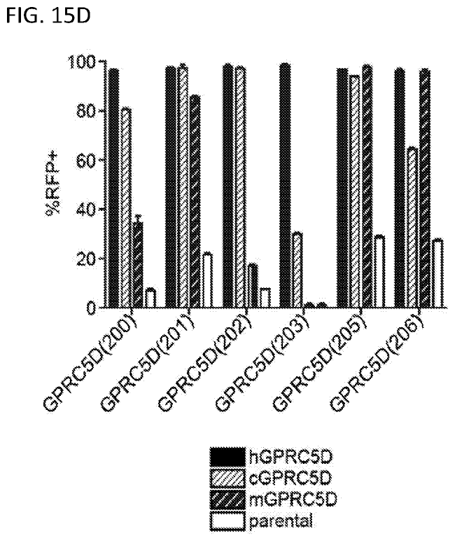

To evaluate potential activation of GPRC5D-targeted CAR T cells by essential normal cells, primary human T cells were genetically modified to express the GPRC5D-203 V_{L}/V_{H}, long spacer CAR and co-cultured with a panel of isolated primary human cell types, after which cytokine release was measured. While co-culture of T cells expressing the GPRC5D-203 V_{L}/V_{H}, long spacer CAR with positive control OPM2 MM cells led to substantial IFNγ, IL-2, and TNFα release, quantities of cytokines in the media after co-culture with any of the twenty normal tissue types investigated were minimal (FIGS. 15A-15C). For example, IFNγ was 2600-fold higher after OPM2 co-culture when compared to the highest value after co-culture with cells isolated from normal tissue.

B. On-Target/Off-Tumor Effects in Reporter Cells

To study on-target, non-tumor effects in mice and non-human primates, anti-GPRC5D CARs were identified that were cross-reactive for murine GPRC5D (mGPRC5D) and/or cynomolgus GPRC5D (cGPRC5D), which have 82% and 96% amino acid homology to human GPRC5D (hGPRC5D), respectively. K562 cells were transduced to express human (hGPRC5D), cGPRC5D or mGPRC5D. The Jurkat Nur77-RFP activation reporter line described above was transduced with polynucleotides constructs encoding CARs described in Example 3. CAR activation was measured as % RFP+ cells of the GFP+ CAR-transduced cells after co-culture of each target cell type with each anti-GPRC5D CAR-expressing reporter cell at an effector:target ratio of 1:1. Reporter cells were also incubated with parental K562 cells as a negative control.

The results are provided in FIG. 15B for exemplary CARs containing a long spacer and an scFV selected from GPRC5D-200 V_{L}/V_{H}, GPRC5D-201 V_{L}/V_{H}, GPRC5D-202 V_{H}/V_{L}, GPRC5D-203 V_{L}/V_{H}, GPRC5D-205 V_{L}/V_{H}, and GPRC5D-206 V_{H}/V_{L}. Cells expressing a CAR containing GPRC5D-203 V_{L}/V_{H} scFv were not cross-reactive to either mGPRC5D or cGPRC5D. Cells expressing a CAR containing GPRC5D-205 V_{L}/V_{H} scFv were cross-reactive to mGPRC5D and cGPRC5D, and the other 4 CARs shown in FIG. 15D bound cGPRC5D.

C. On-Target/Off-Tumor Effects in a Mouse Model

Human CAR T cells expressing a CAR incorporating the mGPRC5D cross-reactive GPRC5D-205 V_{L}/V_{H} scFv, long spacer CAR or non-cross reactive GPRC5D-203 V_{L}/V_{H} scFv, long spacer CAR, were tested in a murine tumor model. OPM2-ffLuc cells were injected into NOD scid gamma (NSG™) to generate an OPM2 human myeloma cell line xenograft model as described above. Mice were injected with 3×10⁶ human CAR+ T cells 14 days post OPM2 MM cell line. Untreated and mock-transduced T cell treatment served as controls. Clinical observations, including body temperature and weight, were taken daily for 13 days, and tumor burden (assessed by OPM2 BLI) was measured every 2-3 days for 14 days.

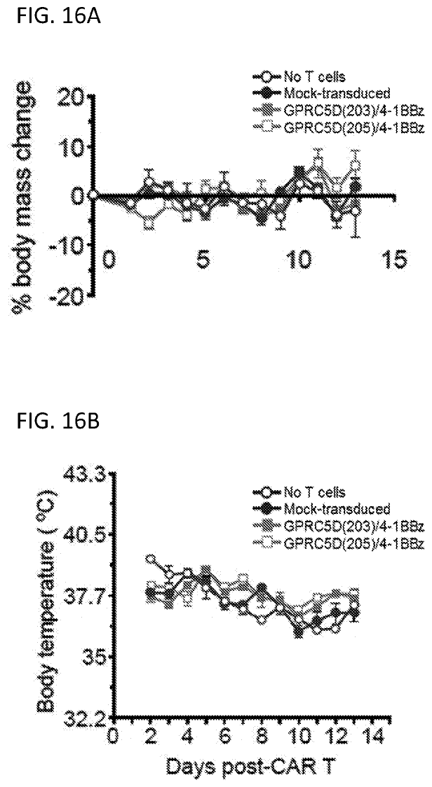

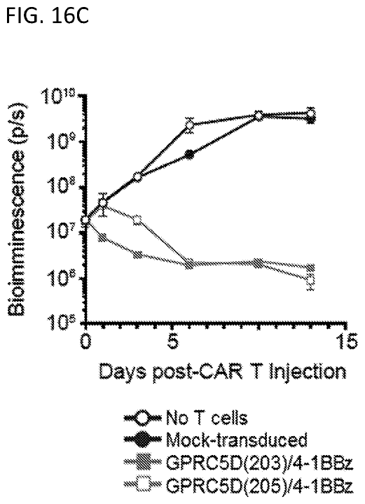

Cross-reactive GPRC5D-205 V_{L}/V_{H} and non-cross-reactive GPRC5D-203 V_{L}/V_{H} CAR-containing T cells were well tolerated, as noted by weight and body temperature curves that did not differ significantly between non-treated and the different CAR T cell treated groups (FIG. 16A and FIG. 16B, respectively). Further, both cross-reactive GPRC5D-205 V_{L}/V_{H} and non-cross-reactive GPRC5D-203 V_{L}/V_{H} CAR-containing T cell therapies showed activity in reducing OPM2 tumor burden (FIG. 16C). In addition, no fur loss or other overt clinical sign of toxicity were observed for any of the treatment conditions.

D. On-Target/Off-Tumor Effects in a Non-Human Primate (NHP) Model

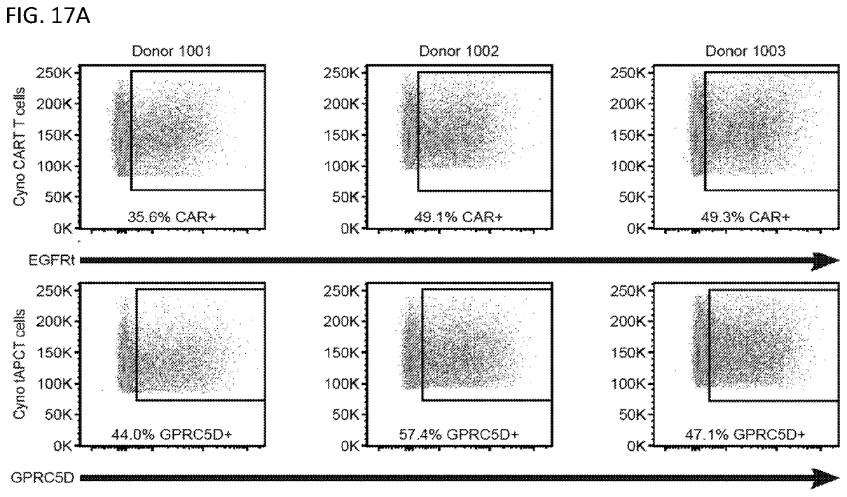

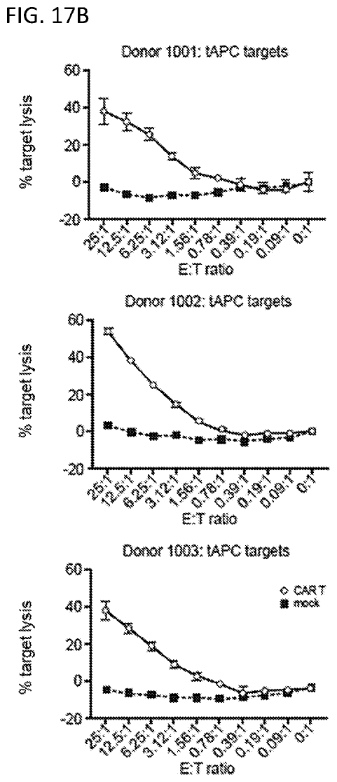

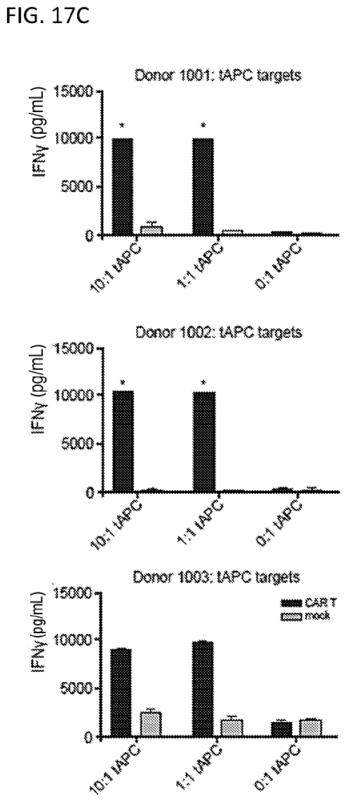

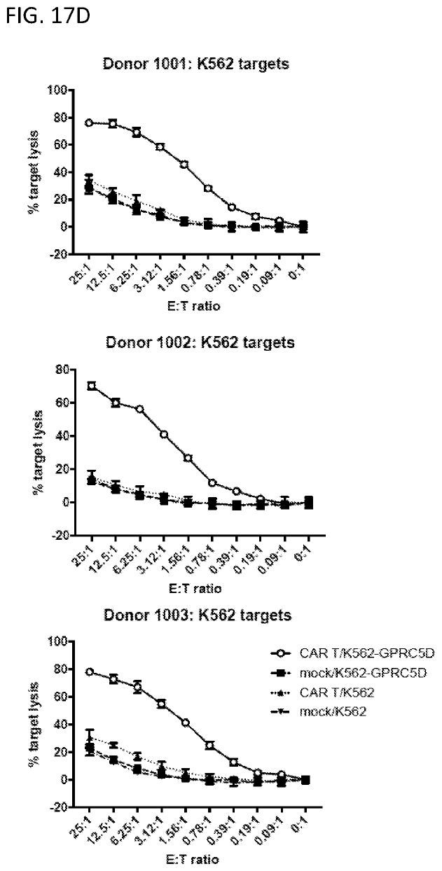

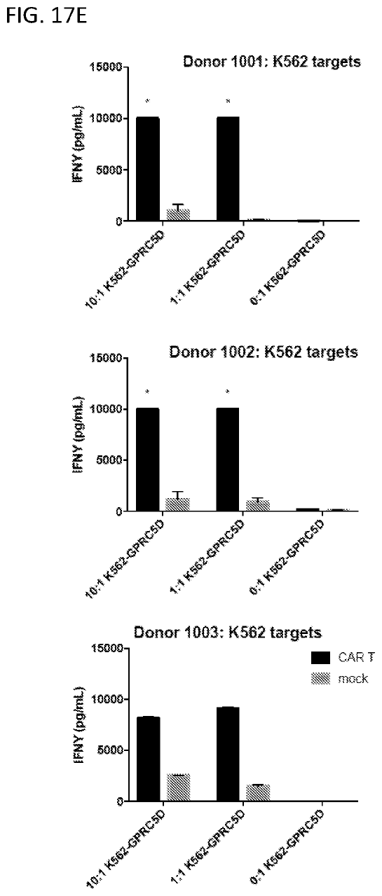

Human CAR T cells expressing a CAR incorporating the cGPRC5D cross-reactive GPRC5D-202 V_{H}/V_{L} scFv, long spacer CAR were tested in a primate tumor model. T cells were harvested and isolated from three (3) male non-human primates (NHPs; cynomolgus monkeys), designated 1001, 1002 and 1003, and transduced with retrovirus containing polynucleotide encoding cGPRC5D cross-reactive GPRC5D-202 V_{H}/V_{L} CAR. As shown in Table E11A, the transduction rate of the CAR into NHP T cells was 36-49% of CD3+ cells. After gene transfer, NHP T cells remained viable (80-92%). FIG. 17A provides representative FACs analysis of CAR expression as measured based on co-expressed truncated receptor used as a surrogate marker, which was separated from the encoding CAR nucleic acid sequence by a ribosome skip element.

TABLE E11A
Properties of Cells

| | | | | | | | | | Projected |
|---|---|---|---|---|---|---|---|---|---|
| | | | | | | | | | CAR/tAPC+ |
| | | | | | | | | % Cas3+ or | Caspase3− |
| Donor | Construct | Viability | % TE | % CD4 | % CD8 | % Tcm | % Teff | AnnV+ | cells/kg dose |
| 1001 | CAR | 80.1 | 35.6 | 44.8 | 50.4 | 90.0 | 7.8 | 9.4 | 1.8e7 |
| | tAPC | 75.0 | 44.0 | 60.8 | 34.9 | 86.6 | 10.7 | 8.7 | 5.2e6 |
| 1002 | CAR | 92.1 | 49.1 | 48.5 | 60.5 | 93.8 | 3.8 | 9.2 | 1.37e7 |
| | tAPC | 91.4 | 54.4 | 41.3 | 55.4 | 95.2 | 2.7 | 8.9 | 1.37e7 |
| 1003 | CAR | 91.2 | 49.3 | 41.7 | 55.4 | 89.2 | 7.5 | 8.1 | 1.25e7 |
| | tAPC | 87.9 | 47.1 | 38.2 | 55.7 | 90.9 | 6.2 | 11.7 | 1.66e7 |

Tcm (T central memory; CD95+CD28+);
Teff (T effector memory; CD95+CD28−)

The NHP T cells were evaluated for cytolytic activity and cytokine production against autologous target antigen presenting cells (tAPCs) and K562-cGPRC5D cells. As shown in FIGS. 17B-E, the NHP T cells were determined to be functional, exhibiting cytolytic activity (FIGS. 17B and D) and interferon gamma release (approaching 10,000 pg/mL supernatant after co-culture with cells expressing GPRC5D at a 1:1 E:T ratio) in response to exposure to the antigen (FIGS. 17C and E).

The three (3) NHPs (i.e., 1001, 1002 and 1003, from which the CART cells were generated above) were pre-treated with lymphodepleting conditioning cyclophosphamide (40 mg/kg) at 2 and 4 days prior to CAR T cell injection, and then administered 10×10⁶ CAR+ caspase3 autologous cynomolgus T cells/kg, generated as described above. Clinical observations, including body temperature and weight were made every 2-7 days, starting 8 days prior to T cell injection and continuing for 21 days post T cell injection. All three NHPs were also exposed to the skin irritant imiquimod 4 days prior to CAR T cell injection because of the concern for hair follicle expression of GPRC5D, in order to increase the sensitivity of detecting possible toxicity. Four days after CAR T cell injection, each NHP received 10×10⁶ cGPRC5D+ caspase-autologous artificial antigen presenting T cells/kg to act as an antigen reservoir and boost CAR T cell expansion. At 21 days post CAR T cell administration, the NHPs were sacrificed for gross and histologic examination.

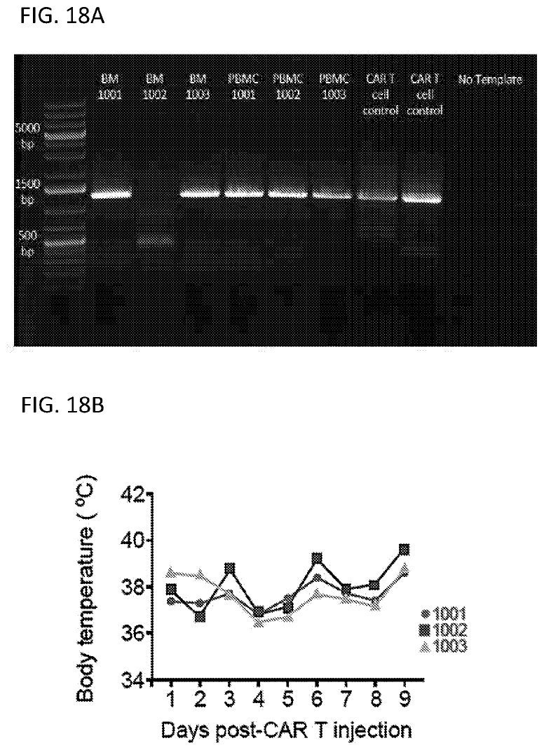

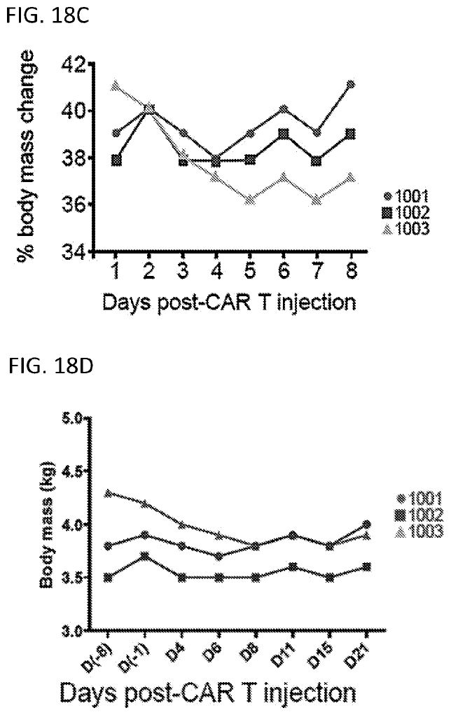

PCR for the DNA encoding the GPRC5D-202 V_{H}/V_{L}-containing CAR was used to monitor T cell persistence at day 21 after infusion in the peripheral blood and bone marrow. Total RNA was isolated from 2 to 4 curls (20 μm thickness) of FFPE cell pellets or tissue samples, and cDNA was synthesized using a cDNA Synthesis kit. PCR was carried out using quantified primers as follows: 0.25 μM each of GPRC5D forward primer (5′-ACTGCATCGAGTCCACTGGAGA-3′; SEQ ID NO:253), and reverse primer (5′-GGATCTTTCGCATGAGGAAGAG-3′, SEQ ID NO:254), and 0.25 μM each of housekeeping gene β-actin forward primer (5′-AGCATCCCCCAAAGTTCAC-3′; SEQ ID NO:255) and reverse primer (5′-AAGGGACTTCCTGTAACAACG-3′, SEQ ID NO:256). PCR reactions were carried out in duplicate in 384-well PCR plates. CAR transduced NHP T cells were used as a positive control. As shown in FIG. 18A, post-infusion CAR T cell persistence was detected in the peripheral blood in 3/3 NHPs and in the bone marrow in 2/3 NHP at time of sacrifice for gross and histologic examination.

GPRC5D-202 V_{H}/V_{L}-containing CAR T cell therapy was well tolerated as determined by clinical observation, body temperature (FIG. 18B), and weight curves (FIGS. 18C, D) that remained stable after CAR T cell treatment. A summary of microscopic findings is provided in Table E11B. There were no increases in IFN-gamma, IL-6 or other pro-inflammatory cytokines, as well as no relevant changes in clinical chemistry observed. There was no fur loss or other clinical sign of damage to the skin, lungs, or other tissues clinically or pathologically noted. Together these data are consistent with an observation that the administration of GPRC5D specific CAR T cells in the NHP model did not cause any overt toxicity or tissue pathology related to the presence of the CAR T cells.

TABLE E11B
Summary of Microscopic Findings at Day 21

| | Group |
|---|---|
| | Males |
| | No. animals examined |
| | 1 |
| | 3 |
| Spleen | |
| Increased cellularity, lymphoid, white pulp | (2)^{a} |
| Mild | 1001^{b}, 1002 |
| Lymph node mesenteric | |
| Increased number/size, lymphoid follicle, | (3) |
| germinal center | |
| Minimal | 1001 |
| Mild | 1002, 1003 |
| Liver | |
| Fibrosis, focal with pigmented macrophages | (2) |
| Minimal | 1002, 1003 |
| Single cell necrosis, hepatocellular, focal | (1) |
| Minimal | 1002 |
| Lung, left diaphragmatic lobe | |
| Embolus, nuclear material, capillary | (1) |
| Minimal | 1001 |

Severity grades for which there were no findings were omitted from the table
^{a}Numbers in parentheses represent the number of animals with the finding
^{b}Animal identification numbers

### Example 12: Evaluation of GPRC5D-Targeted CAR T Cells in a Murine BCMA Antigen Loss Model

A murine model for BCMA-negative multiple myeloma was generated and used to assess the effects of GPRC5D-targeted CAR T cells when administered to animals of the model.

To generate BMCA-knockout tumor cells, GFP/Luciferase Cas9-expressing OPM2 cells were transduced with a BCMA single guide RNA (sgRNA)-containing lentivirus. The BCMA sgRNA, directed against a target sequence set forth in SEQ ID NO:291, was cloned into the pLentiV2 plasmid, and subsequently co-transfected with packaging plasmids (Pax2 and Vsvg) into 293T cells. Transduction was carried out in the presence of 10 μg/mL polybrene. Transduced cells were selected with 4 μg/mL puromycin for 10 days and single cells were played in a 96-well plate. Individual colonies were expanded and stained with anti-BCMA antibody to confirm BCMA knockout. Clones of confirmed BCMA knockout OPM2-GL-Cas9 cells were further expanded for experiments.

To model the presence of BCMA antigen-negative tumor cells in the multiple myeloma setting (which could potentially lead to relapse or progression following treatment with a BCMA-targeted agent), NSG mice were injected with a mixed population of unmanipulated OPM2WT (about 75%) and GFP/ffLuc+ CRISPR-mediated OPM2^{BCMA-KO} (about 25%) (1×10⁶ total OPM2 cells) on day 0. The population of OPM2 cells was allowed to engraft and expand for 7 days. On day 8, mice were injected with 4×10⁶ CAR T cells targeting either CD19 or BCMA. On day 16, mice previously injected with BCMA-targeting CAR T cells were injected with 4×10⁶ T cells expressing an anti-BCMA CAR or the GPRC5D-203 V_{L}/V_{H}, long spacer CAR.

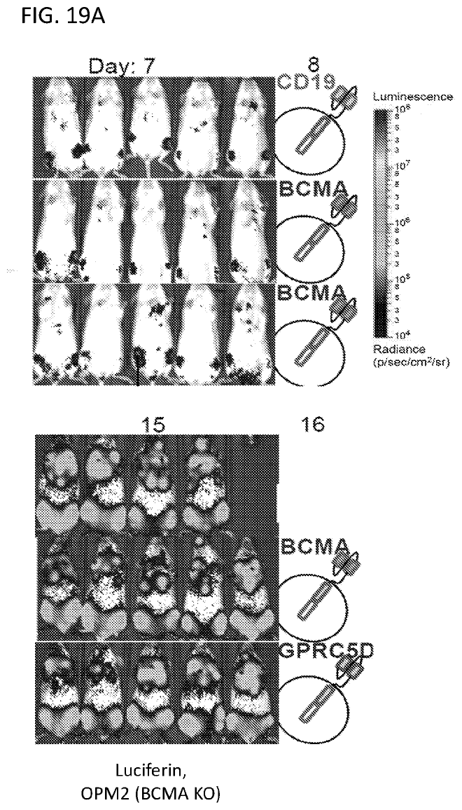

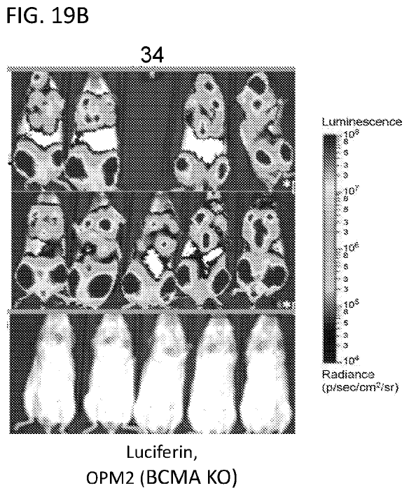

BLI was used to image mice to detect OPM2^{BMCAKO} cells (the population of cells expressing GFP/ffLUC). Imaging of mice on day 34 revealed that the OPM2^{BMCAKO} sub population progressed in mice that had received BCMA-targeting CAR T cell injections on days 8 and 16. By contrast, mice that had received BCMA-targeting CAR T cells on day 8 and GPRC5D-targeting CAR T cells on day 16 did not exhibit luminescence on day 34 (FIGS. 19A and 19B). The results were consistent with a finding that anti-GPRC5D CAR T-cells can exhibit anti-tumor activity in multiple myeloma, even in the context of tumor cells not expressing BCMA antigen or not responsive to BCMA-directed CAR-T cells; tumor escape could be rescued by the GPRC5D-203 V_{L}/V_{H} CAR-expressing T cells.

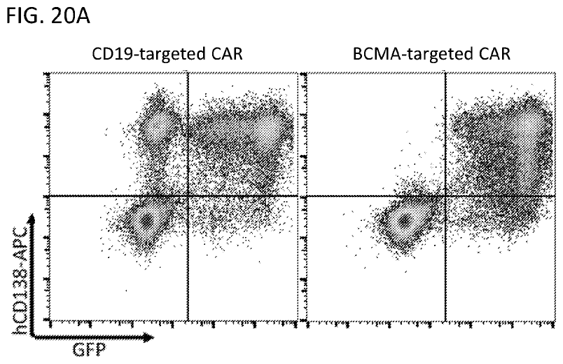

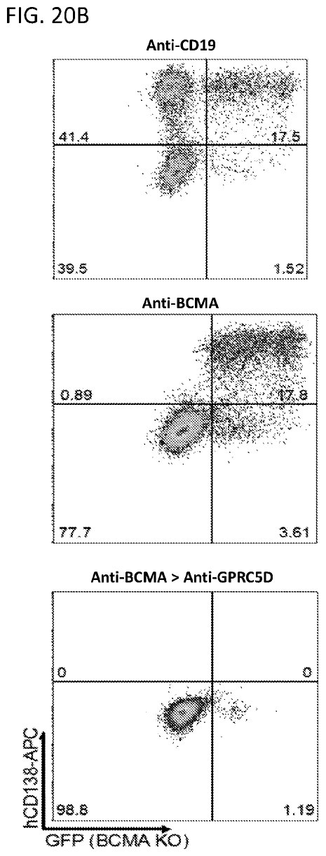

The percent of CD138hi/GFP+ cells in bone marrow was analyzed by flow cytometry on day 35 to assess the composition of OPM2 cells (hCD138+; WT: GFP−; BCMA-KO: GFP+) in the bone marrow of progressing mice. OPM2 (hCD138+) composition (WT:GFP−; BCMA^{KO}:GFP+) was assessed from flushed bone marrows from mice treated with CD19-, BCMA-, or GPRC5D-targeting CAR T cells, (FIGS. 20A and B; representative of 3 mice/arm; n=2 replicate experiments with comparable results). Live and dead gating was performed but is not shown. The majority of bone marrow cells from mice that had received an injection of BCMA-targeting CAR T cells on day 8 were CD138hi/GFP+(FIGS. 20A and B) at day 35. The results were consistent with the ability of BCMA-targeting CAR T cells to eradicate OPM2′ cells despite the persistence and/or progression of the OPM2^{BCMA-KO} sub-population of tumor cells.

### Example 13: Generation of Dual-Targeting Chimeric Antigen Receptor (CAR)-Engineered T Cell Compositions Targeting GPRC5D and BCMA

Dual-targeting T cell compositions were generated in which the compositions contained cells expressing chimeric antigen receptors (CARs) for dual-targeting of BCMA and GPRC5D. Two separate engineered T cell compositions were generated in which one was engineered to express the anti-BCMA CAR and the other was engineered to express the anti-GPRC5D CAR and the separate cell compositions were pooled.

The exemplary anti-BCMA CAR contained an scFv antigen-binding domain able to bind cells expressing BCMA (e.g. BCMA-55, V_{L}/V_{H} scFv (SEQ ID NO: 241); an immunoglobulin hinge-CH2-CH3 set forth in SEQ ID NO: 17, encoded by the sequence set forth in SEQ ID NO:73; a human CD28-derived transmembrane domain (e.g. SEQ ID NO: 18); a human 4-1BB-derived intracellular signaling domain (e.g. SEQ ID NO: 19); and a human CD3zeta-derived intracellular signaling domain (e.g. SEQ ID NO: 20).

The exemplary anti-GPRC5D CAR contained an scFv antigen-binding domain able to bind GPRC5D (e.g. GPRC5D-203 V_{L}/V_{H} scFv (SEQ ID NO: 8); an immunoglobulin hinge-CH2-CH3 set forth in SEQ ID NO: 17, encoded by the sequence set forth in SEQ ID NO:73; a human CD28-derived transmembrane domain (e.g. SEQ ID NO: 18); a human 4-1BB-derived intracellular signaling domain (e.g. SEQ ID NO: 19); and a human CD3zeta-derived intracellular signaling domain (e.g. SEQ ID NO: 20).

A. Generation of Pooled T Cells Expressing Anti-GPRC5D and Anti-BCMA CARs

Polynucleotides encoding each individual CAR were linked to a downstream ribosomal skip element (such as a self-cleaving T2A sequence (sequence set forth in SEQ ID NO:44, encoding the amino acid sequence set forth in SEQ ID NO: 37) and cloned into a lentiviral expression vector. T cell compositions were separately engineered to express either the anti-BCMA CAR or anti-GPRC5D CAR. For each, either a Nur77-RFP Jurkat T cell line or primary T cells isolated by immunoaffinity-based enrichment from leukapheresis samples from human donor subjects were transduced with one of the lentiviral vectors containing nucleic acid encoding either the anti-BCMA CAR or the anti-GPRC5D CAR. After transduction, and in some cases, expansion, T cells were stained with an antibody specific for the respective CAR and analyzed by flow cytometry, confirming transduction of cells and expression of the CAR.

Prior to use, harvested primary human T cells transduced with either the anti-BCMA CAR or the anti-GPRC5D CAR were pooled at a 1:1 ratio of anti-BCMA CAR-expressing cells to anti-GPRC5D CAR-expressing cells (“αGPRC5D and αBCMA pooled cells”).

### Example 14: Assessment of the Anti-BCMA CAR

The exemplary anti-BCMA CAR described in the example above was assessed for antigen-independent (tonic) signaling, as well as proliferation and activation in the presence of BCMA expressing and non-expressing cells.

A. Antigen-Independent (Tonic Signaling)

The Nur77-RFP reporter cell line described in previous examples was transduced with a viral vector encoding anti-CD19 CAR (control), or the exemplary anti-BCMA CAR as described in Example 13, with the exception that the surrogate marker for transduction was super-fold green fluorescent protein, sfGFP. In this model, tonic signaling was indicated by RFP expression in the absence of BCMA antigen stimulation. The CAR-expressing cells were incubated without antigen stimulation to assess the degree of antigen-independent (tonic) signaling for 3 days and evaluated for the expression of RFP by flow cytometry.

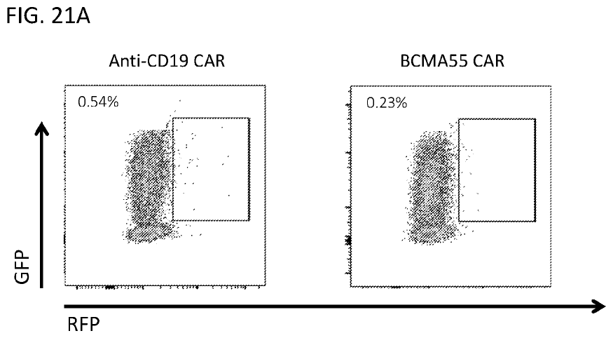

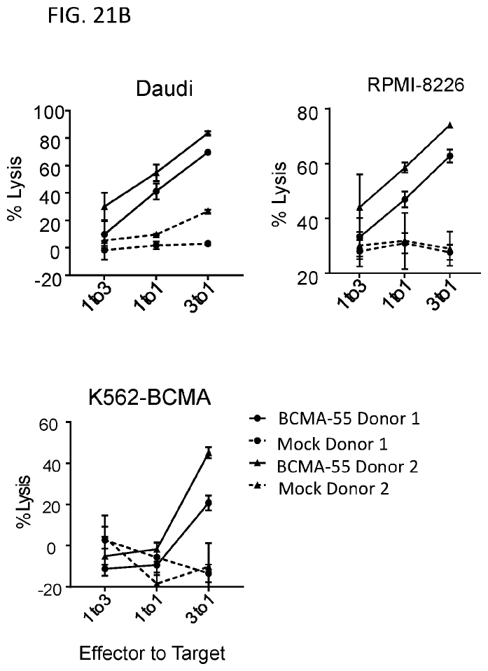

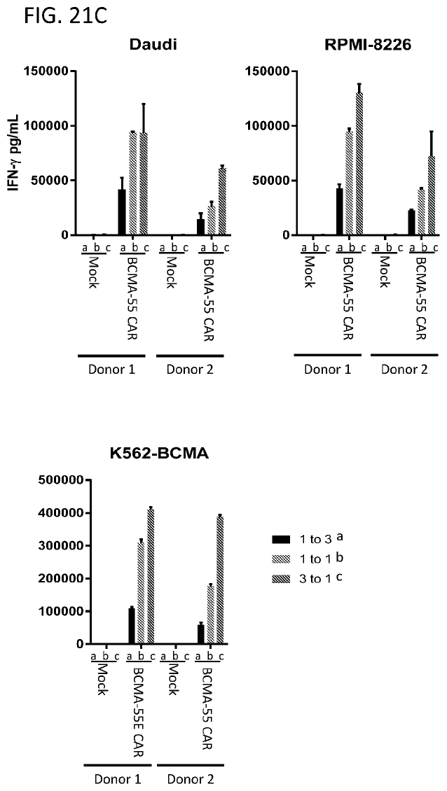

As shown in FIG. 21A, the exemplary anti-BCMA CAR-expressing cells exhibited low RFP expression in the absence of antigen stimulation, as determined by the percentage of RFP+ cells (indicative of tonic reporter activation) among CAR-expressing cells (indicated by GFP+ cells).

B. Proliferation and Activation of BCMA-Targeted CAR T Cells

Cytolytic activity, cytokine release, and proliferation were assessed following incubation of exemplary anti-BCMA CAR-expressing T cells with BCMA-expressing cells that expressed different levels of BCMA. All activity was evaluated in the presence or absence of soluble BCMA.

A 1:1 ratio of CD4+ and CD8+ primary T cells, harvested from two human donors (D #1 and D #2), were stimulated with CD3/CD28 beads and transduced with a lentiviral vector to stably express BMCA-55 CAR. Transduced cells were cultured in the presence of BCMA-expressing target cells at an E:T ratio of 1:3, 1:1 or 3:1. Mock-processed T cells from the same donors were also mixed with target cells for use as a control. The BCMA+ target cells, Daudi, RPMI-8226, and K562-BCMA cell, exhibited different levels of BCMA antigen-density of the surface (antigen density: Daudi (<1000 BCMA molecules/cell)<RPMI-8226<K562-BCMA) and were stained with carboxyfluorescein succinimidyl ester (CFSE) prior to incubation with the T cells. An equal number of target-negative cells, not expressing BCMA and stained with cell trace violet (CTV), were also included in the cultures with the T cells and BCMA+ target cells. After a 24 hour incubation, the remaining BCMA+vs BCMA-target cells were measured by flow cytometry, and the degree of target cell lysis, indicative of cytotoxicity, was assessed.

The anti-BCMA CAR-expressing T cells displayed similar cytolytic activity when cultured with target cells, regardless of BCMA expression levels (FIG. 21B). Additionally, similar results were observed for target cells (NCI-H929) expressing a greater than 100,000 molecules per cell. Mock-processed T cells did not show activity against any of the BCMA+ target cell lines. Target cells negative for BCMA expression were not lysed by the anti-BCMA CAR T cells from any of the donors tested (data not shown).

The supernatants following the incubation were analyzed for accumulated IFN-γ, TNF-α, and IL-2 cytokines. Data were consistent with a conclusion that the anti-BCMA CAR T cells had released a range of cytokines following engagement with BCMA-expressing target cells; with the level of cytokines released generally corresponding with increasing level of antigen (i.e., Daudi<RPMI 8226<K562-BCMA). Results for IFN-γ are shown in FIG. 21C; similar data were observed for TNF-α and IL-2 (data not shown). Anti-BCMA CAR T cells did not release cytokines in response to BCMA-negative targets, nor did they express cytokines without any target cells present, demonstrating specificity for BCMA+ target cells and lack of tonic signaling.

Activity of the anti-BCMA CAR-expressing T cells in the presence vs. absence of soluble BCMA was assessed. T cells expressing the exemplary anti-BCMA CAR were co-cultured with RPMI-8226 tumor cells, with recombinant BCMA-Fc, or with cell culture supernatant derived from NCI-H929 multiple myeloma cells (BCMA-secreting cell line, the supernatant containing soluble BCMA). Neither tumor-cell lysis nor cytokine production was observed to be affected by any of the concentrations of NCI-H929-derived soluble BCMA (up to 1000 ng/mL). Both tumor-cell lysis and cytokine production were only minimally decreased at similarly high physiological levels of recombinant BCMA.

Proliferation in response to BCMA was measured in anti-BCMA CAR-expressing T cells and mock-processed T cells. Transduced T cells were labeled with cell trace violet (CTV) and cultured in the presence of BCMA-positive target cells, BCMA-negative target cells, or no cells, at an effector to target (E:T) ratio of 1:1 for 72 hours. Proliferation was measured by flow cytometry. Proliferation of T cells (CD4+ and CD8+ T cells) was observed only for anti-BCMA CAR-expressing T cells in response to incubation with BCMA-positive target cells (data not shown).

### Example 15: In Vitro Assessment of Engineered T Cell Compositions Containing Anti-GPRC5D and Anti-BCMA CAR-Expressing Cells

A. Antigen-Dependent Activity in the Presence of BCMA or GPRC5D KO Cells

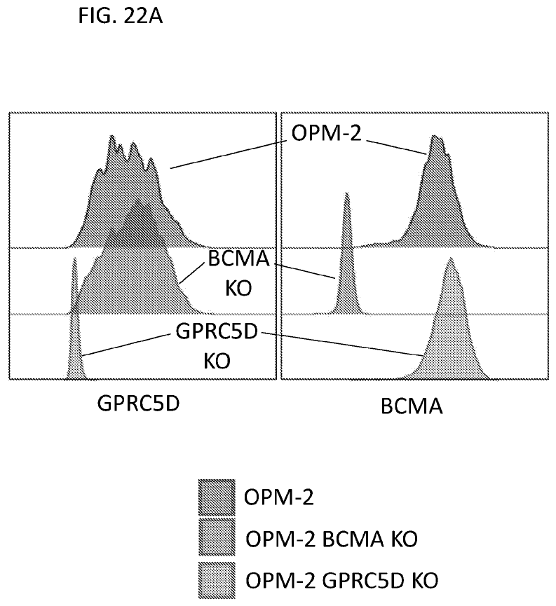

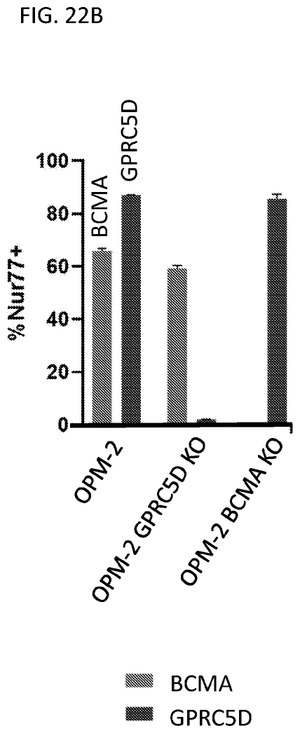

To understand the specificity of the anti-BCMA and anti-GPRC5D CARs, either GPRC5D or BCMA was knocked out from OPM2 cells as previously described in Examples 5 and 12, respectively. As shown in FIG. 22A, flow cytometric analysis confirmed a lack of GPRC5D expression in the OPM2 GPRC5D KO cells and a lack of BCMA expression in the OPM2 BCMA KO cells. OPM2 wild-type (OPM2 WT) cells served as a control, showing expression of both GPRC5D and BCMA.

Jurkat Nur77-RFP reporter cell compositions containing cells engineered to express only the anti-GPRC5D CAR (αGPRC5D) or only the anti-BCMA CAR (αBCMA) were assessed for antigen-specific activity. The Jurkat Nur77-RFP report cell compositions were co-cultured with OPM2 WT, OPM2 GPRC5D KO, or OPM2 BCMA KO cells at a 1:1 ratio for 20 hours. Antigen-specific stimulation of the CARs expressed by the cell compositions was assessed by measuring changes in RFP expression by flow cytometry.

As shown in FIG. 22B, activation of Jurkat Nur77-RFP reporter cells expressing either only the GPRC5D-targeting CAR (αGPRC5D) or only the BCMA-targeting CAR (αBCMA) was not observed when they were co-cultured with OPM2 cells in which GPRC5D or BCMA was knocked out, respectively. This result is consistent with a finding that the anti-BCMA and anti-GRPC5D CARs are specific for their respective antigens.

### Example 16: In Vivo Assessment of Dual-Targeting Pooled Cell Compositions Containing Cells Expressing GPRC5D- and BCMA-Targeting CARs

Various murine multiple myeloma models were generated and used to assess the in vivo effects of T cell compositions containing (1) cells engineered to express only the anti-GPRC5D CAR (αGPRC5D), (2) cells engineered to express only the anti-BCMA CAR (αBCMA), or (3) pooled cells containing cells separately engineered to express either the anti-BCMA CAR or the anti-GPRC5D CAR pooled at 1:1 ratio of anti-BCMA CAR-expressing cells to anti-GPRC5D CAR-expressing cells (αGPRC5D and αBCMA pooled cells).

A. Expression of GPRC5D and BCMA by Multiple Myeloma Cell Lines

The relative mRNA and protein expression levels of GPRC5D and BCMA were assessed among various multiple myeloma cell lines prior to creation of the murine multiple myeloma models.

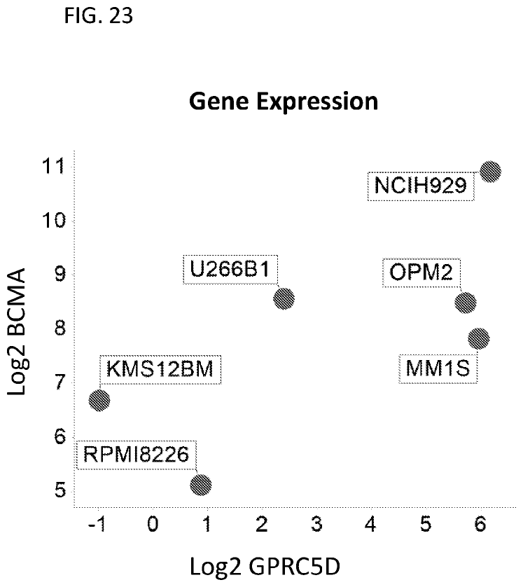

mRNA expression levels for GPRC5D and BCMA, as provided by the Cancer Cell Line Encyclopedia (CCLE), were determined by RNA sequencing (RNA-seq) in the following multiple myeloma cell lines: KMS12BM, RPMI8226, U266B1, MM.1S, OPM2, and NCI-H929. As shown in FIG. 23, the KMS12BM and RPMI8226 cell lines exhibited the lowest levels of expression for both genes among all cell lines analyzed. The U266B1 cell line exhibited moderate levels of GPRC5D and BCMA gene expression, compared to the other cell lines. The MM.1S and OPM2 cell lines exhibited high levels of GPRC5D gene expression and moderate levels of BCMA gene expression, compared to the other cells. Finally, NCI-H929 exhibited the highest gene expression levels for GPRC5D and BCMA among all of the cell lines tested.

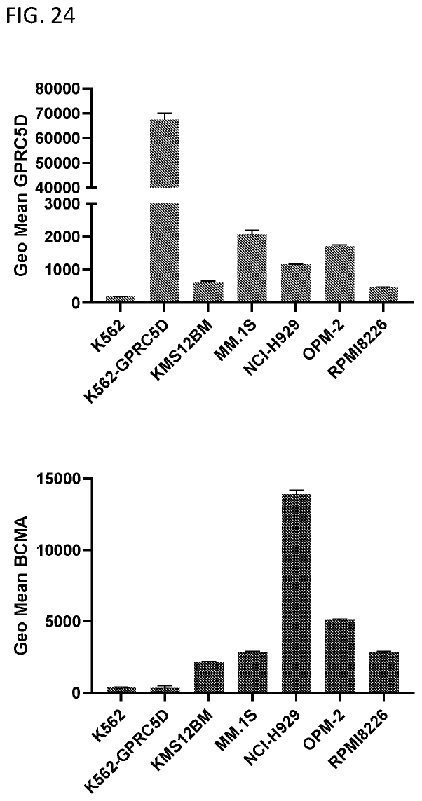

As shown in FIG. 24, protein expression levels for GPRC5D and BCMA were additionally analyzed in the following cell lines: K562 parental cells, K562 cells engineered to express GPRC5D, KMS12BM, MM.1S, NCI-H929, OPM2, and RPMI8226. 5×10⁵ total cells were co-labeled with an anti-BCMA or an anti-GPRC5D antibody, and a viability marker, and expression was determined by flow cytometry. In terms of GPRC5D protein expression, the K562 cells engineered to express GPRC5D served as a positive control, demonstrated the highest level of GPRC5D expression, while the parental K562 cells serving as the negative control expressed negligible levels of GPRC5D. In general agreement with the mRNA expression data, KMS12BM and RPMI8226 expressed relatively low levels of GPRC5D protein. NCI-H929 expressed a moderate level, while MM.1S and OPM-2 expressed relatively high levels, of GPRC5D protein. In terms of BCMA protein expression, KMS12BM, MM1S, and RPMI8226 all expressed low to moderate levels of BCMA protein, while OPM-2 expressed a high level of BCMA protein. Still, NCI-H929 cells expressed the highest level of BCMA protein.

Taken together, the results are consistent with a finding that the RPMI8226 cell line exhibits relatively low GPRC5D expression, while the OPM2 cell line exhibits relatively high expression of GPRC5D.

B. Assessment of T Cell Compositions Expressing Anti-BCMA and Anti-GPRC5D CARs in an OPM2 Murine Xenograft Model

Dual BCMA- and GPRC5D-targeting primary human T cell compositions containing (1) cells expressing only the anti-GPRC5D CAR, (2) cells expressing the anti-BCMA CAR, or (3) pooled cells separately engineered to express either the anti-BCMA CAR or the anti-GPRC5D CAR pooled at 1:1 ratio of anti-BCMA CAR-expressing cells to anti-GPRC5D CAR-expressing cells (αGPRC5D and αBCMA pooled cells) were tested in a murine tumor model with tumors expressing either or both of the target antigens.

To generate the tumor model, GPRC5D and BCMA were separately knocked out in OPM2-rFLuc cells as described in Examples 5 and 12 to generate single knockout GPRC5D and BCMA OPM2 cell lines (“OPM2 GPRC5D KO” and “OPM2 BCMA KO,” respectively). As a control, OPM2-rFLuc cells expressing both GPRC5D and BCMA (OPM2 WT) were compared to the single knockout lines. NOD scid gamma (NSG™) were injected with 2×10⁶ total OPM2, OPM2 GPRC5D KO, or OPM2 BCMA KO cells. At day 14 post-injection (day 0) bioluminescence of the tumors was determined and mice with similar tumor sizes were treated with the primary T cells compositions containing cells engineered to express the anti-GPRC5D CAR (αGPRC5D) or the anti-BCMA CAR (αBCMA), or containing αGPRC5D and αBCMA pooled cells. Mice were injected with one of three doses of the T cell compositions 14 days following injection with OPM2 cells: 5×10⁵ total CAR T cells, 1×10⁶ total CAR T cells, or 2×10⁶ total CAR T cells. Untreated and mock-transduced T cell compositions served as controls.

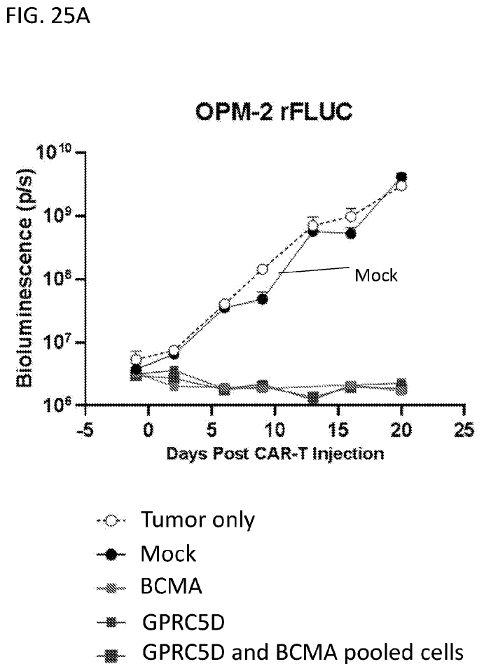

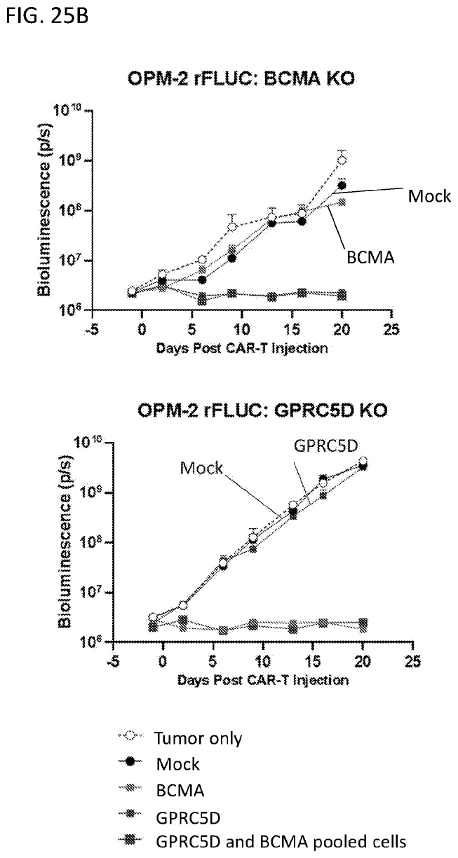

As shown in FIGS. 25A and 25B, in mice administered 3×10⁶ total CAR T cells tumor burden (assessed by OPM2 BLI) remained low in mice treated with the αGPRC5D and αBCMA pooled cells, regardless of the OPM2 cell type injected. By contrast, mice treated only with cells expressing the anti-BCMA CAR (αBCMA) demonstrated increasing tumor burden when injected with OPM2 BCMA KO cells (FIG. 25B, upper panel). Similarly, mice treated only with cells expressing the anti-GPRC5D CAR (αGPRC5D) demonstrated increasing tumor burden when injected with OPM2 GPRC5D KO cells (FIG. 25B, lower panel). This result is in agreement with the general finding that the individual CARs are specific to their targeted antigen.

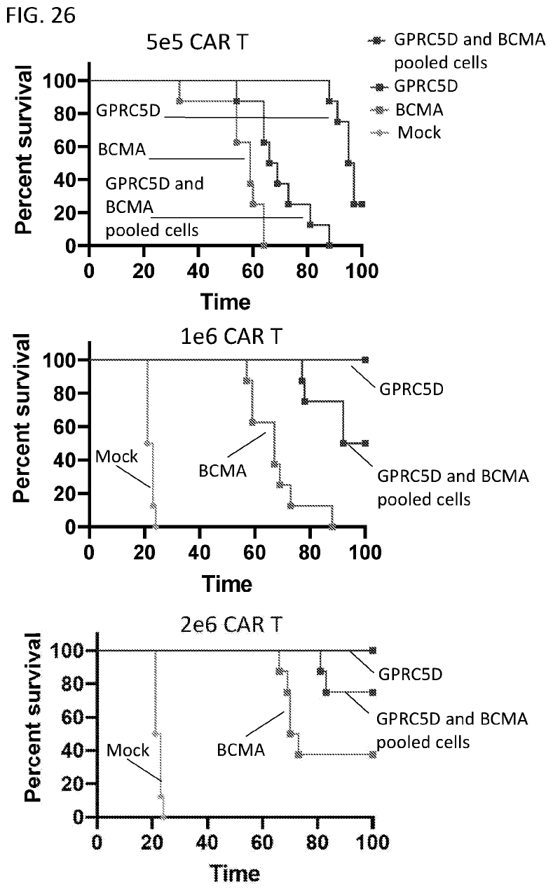

In a similar experiment, NOD scid gamma (NSG™) mice were treated with either 5×10⁵, 1×10⁶, or 2×10⁶ total primary human T cells from compositions containing (1) cells engineered to express the anti-BCMA CAR, (2) cells engineered to express the anti-GPRC5D CAR, or (3) αGPRC5D and αBCMA pooled cells, following injection with OPM2 cells expressing BCMA and GPRC5D. As a control, mice were also treated with mock transduced primary human T cell compositions. Survival was assessed out to 100 days, as shown in FIG. 26. As shown, administration of the T cell composition containing αGPRC5D and αBCMA pooled cells improved the anti-tumor efficacy in this model compared to administration of a T cell composition containing cells only expressing the anti-BCMA CAR.

C. Assessment of T Cell Compositions in an RPMI8226 Murine Xenograft Model

In addition to the OPM2 model, a GPRC5D low expressing RPMI-8226 model was used to evaluate the in vivo effects of human T cell compositions containing cells expressing the anti-GPRC5D CAR (αGPRC5D) or cells expressing the anti-BCMA CAR(αBCMA), or containing both.

A total of 1×10⁷ RPMI8226 cells were injected subcutaneously into NOD scid gamma (NSG™). At about 25 days post-injection of RPMI226 cells, CAR-expressing T cell compositions were administered containing (1) cells engineered to express the anti-GPRC5D CAR (αGPRC5D), (2) cells engineered to express the anti-BCMA CAR (αBCMA), or (3) pooled cells generated at a 1:1 ratio of cells expressing the GPRC5D-targeting CAR to cells expressing the BCMA-targeting CAR (αGPRC5D and αBCMA pooled cells). Mice were treated with one of three doses of the T cell compositions 25 days following injection with the RPMI8226 cells: 1.25×10⁶ total CAR T cells, 2.5×10⁶ total CAR T cells, or 5×10⁶ total CAR T cells. Untreated and mock-transduced T cell treatment served as controls.

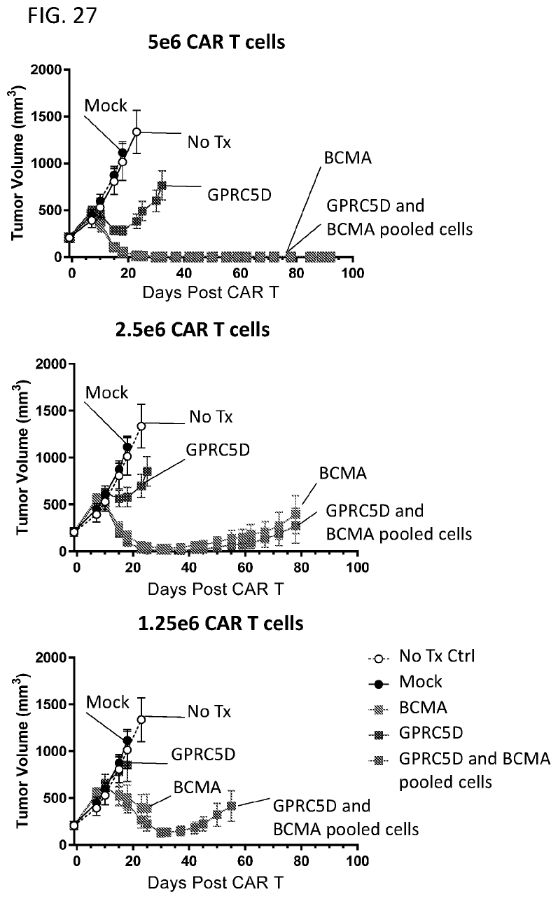

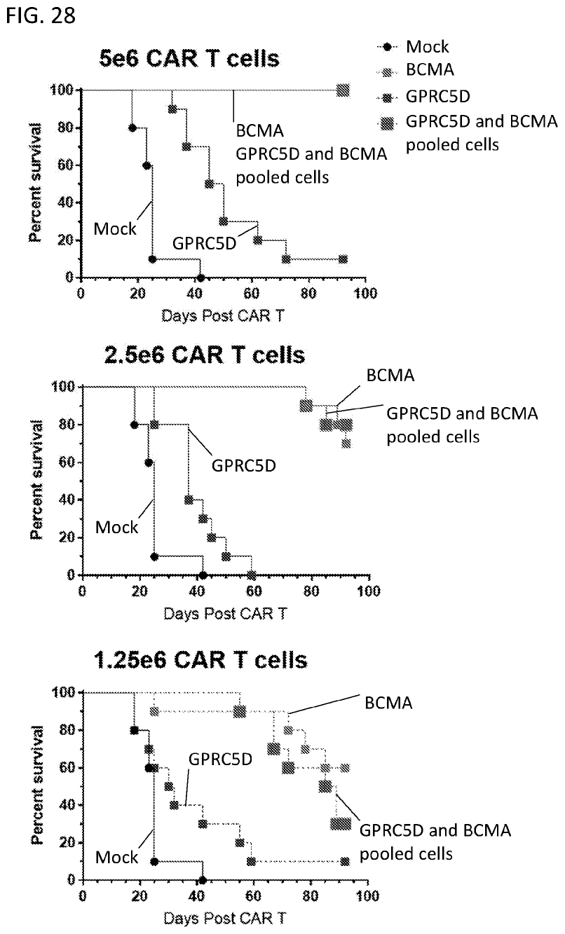

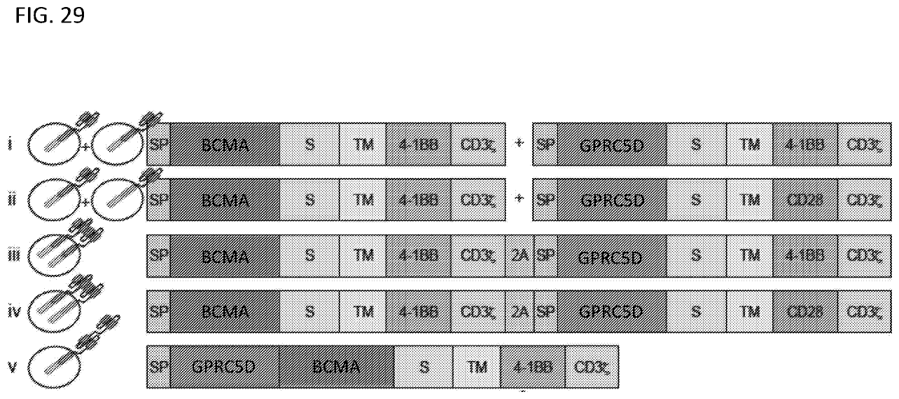

As shown in FIG. 27, tumor volume increased in a dose-dependent manner in mice treated with cells only expressing the anti-GPRC5D CAR. By contrast, mice treated with cells only expressing the anti-BCMA CAR, or with the αGPRC5D and αBCMA pooled cells, demonstrated minimal increases in tumor volume. Notably, the results indicated that at all doses tested (5×10⁶, 2.5×10⁶, and 1.25×10⁶), the αGPRC5D and αBCMA pooled cells had an efficacy similar to T cells only expressing the anti-BCMA CAR, despite that the contribution of the anti-BCMA CAR is half in the pooled T cell composition. This result indicates that the anti-GPRC5D-CAR expressing cells in the pooled composition is contributing in a fashion greater than their single dose. Survival was assessed out to day 100, as shown in FIG. 28. Analysis of survival revealed similar outcomes, in that mice treated with cells only expressing the anti-GPRC5D CAR exhibited decreased survival across time, but at higher doses mice treated with αGPRC5D and αBCMA pooled cells exhibited similar efficacy to mice treated with T cells only expressing the anti-BCMA CAR. These results are in agreement with the general finding that treatment with cells only expressing an anti-GPRC5D CAR may be less efficacious in a low GPRC5D expressing tumor model, but that the anti-GPRC5D CAR may be contributing to anti-tumor efficacy by dual-targeting CAR-expressing cell compositions.

### Example 17: Generation of Bicistronic Anti-BCMA and Anti-GPRC5D CAR-Engineered T Cell Compositions

Primary human T cell compositions were engineered to produce dual-targeting T cell compositions, in which the compositions contained cells expressing chimeric antigen receptors (CARs) for dual-targeting of BCMA and GPRC5D. Five separate engineered T cell compositions were generated, as summarized in FIG. 29. Each generated CAR construct contained an exemplary scFv antigen-binding domain against either GPRC5D (e.g. SEQ ID NO:8) or BCMA (e.g. SEQ ID NO:241); a long spacer (hinge-CH2-CH3, 228aa; SEQ ID NO: 17; with CH2 modifications to limit Fc receptor binding); a human CD28-derived transmembrane domain (SEQ ID NO: 18); either a human 4-1BB-derived intracellular signaling domain (SEQ ID NO: 19) or a human CD28-derived intracellular signaling domain (SEQ ID NO:18); and a human CD3zeta-derived intracellular signaling domain (SEQ ID NO: 20).

In one strategy (shown by (i) and (ii) of FIG. 29), cells expressing a single CAR against either BCMA or GPRC5D were manufactured in parallel and pooled into a single composition containing anti-BCMA CAR-expressing cells and anti-GPRC5D CAR-expressing cells at a 1:1 ratio (GPRC5D and BCMA pooled cells). The anti-BCMA CAR-expressing T cells of the pooled composition were engineered to express an anti-BCMA CAR incorporating a 4-1BB costimulatory domain (SEQ ID NO:312). The anti-GPRC5D CAR-expressing T cells of the pooled composition were engineered to express an anti-GPRC5D CAR incorporating either a 4-1BB co-stimulatory domain (SEQ ID NO:313) or a CD28 costimulatory domain (SEQ ID NO:314). This resulted in two different pooled compositions. The first composition contained cells expressing an anti-BCMA CAR incorporating a 4-1BB co-stimulatory domain and cells expressing an anti-GPRC5D CAR incorporating a 4-1BB co-stimulatory domain (BCMA-41BB and GPRC5D-41BB pooled cells). The second composition contained cells expressing an anti-BCMA CAR incorporating a 4-1BB co-stimulatory domain and cells expressing an anti-GPRC5D CAR incorporating a CD28 co-stimulatory domain (BCMA-41BB and GPRC5D-CD28 pooled cells). Separate compositions containing cells expressing only the anti-BCMA CAR or only the anti-GPRC5D CAR were generated to serve as controls.

In another dual-targeting strategy, shown in (iii) and (iv) of FIG. 29, bicistronic constructs were generated that encoded two separate CARs (anti-BCMA and anti-GPRC5D), in which the nucleotide sequences encoding the anti-BCMA CAR and the anti-GPRC5D CAR were separated by a nucleotide sequence encoding a T2A bicistronic self-cleaving peptide (SEQ ID NO:319, encoding SEQ ID NO:37). The polynucleotides encoding the different CARs were codon diverged to reduce homologous recombination of the nucleotides encoding the anti-BCMA and anti-GPRC5D CARs. In the construct depicted in (iii) both CARs incorporated a 4-1BB costimulatory domain (BCMA-41BB-GPRC5D-41BB; SEQ ID NO:315). In the construct depicted in (iv), the anti-BCMA CAR incorporated a 4-1BB costimulatory domain and the anti-GPRC5D CAR incorporated a CD28 costimulatory domain (BCMA-41BB-GPRC5D-CD28; SEQ ID NO:316).

In a further dual-targeting strategy, a “single stalk” CAR construct was generated as shown in (v), encoding a scFv specific for BCMA and a scFv specific for GPRC5D in tandem (GPRC5D-BCMA-41BB; SEQ ID NO:317), in which the sequences encoding the two scFvs were separated by a flexible linker (SEQ ID NO:320). The exemplary construct incorporated a 4-1BB costimulatory domain (SEQ ID NO:60, encoding SEQ ID NO:19).

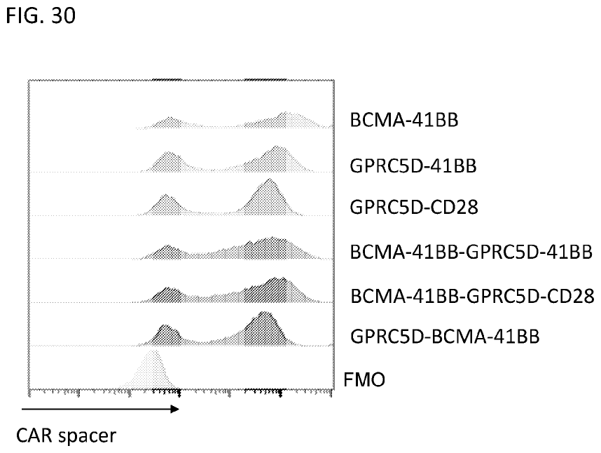

Nucleic acid constructs encoding the various CAR(s) were cloned into retroviral expression vectors for transduction of primary human T cells, substantially as described in Example 3. Following retroviral transduction of cells, expression of the various constructs was assessed by flow cytometric analyses with either anti-GPRC5D CAR or anti-BCMA CAR specific reagents, or an antibody to the spacer domain common to all of the CAR constructs. As shown in FIG. 30, all tested constructs were expressed comparably by primary human T cells. The fluorescence minus one (FMO) negative control did not exhibit expression.

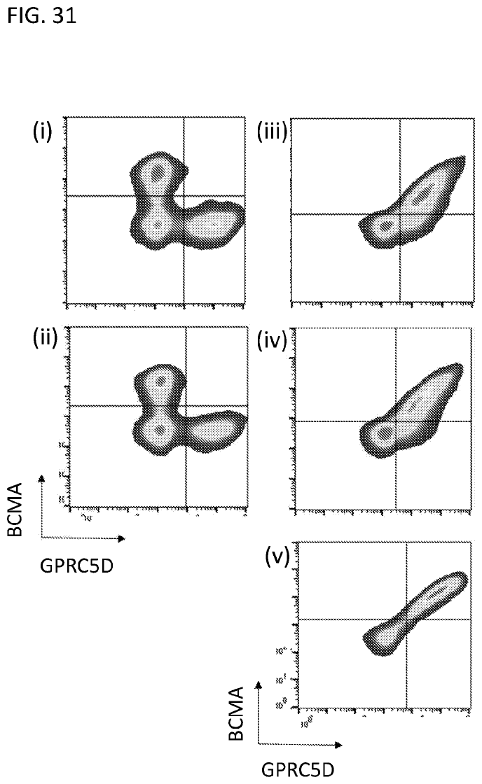

Four to ten days following transduction, primary human T cells were assessed for transduction efficiency using a reagent specific to each CAR. Results are shown in FIG. 31. For all donors assessed, retroviral transduction efficiency for cells engineered with either dual-targeting bicistronic constructs (shown by iii and iv of FIG. 31) or “single stalk” constructs (shown by v of FIG. 31) was determined to be between 50-70%, as compared to cells engineered to express a single CAR as present in the pooled CAR compositions (shown by i and ii of FIG. 31). Results shown are from a representative donor.

### Example 18: In Vitro Assessment of Bicistronic Anti-BCMA and Anti-GPRC5D Chimeric Antigen Receptors

A. Cytotoxicity of T Cell Compositions

CAR-expressing primary human T cells, generated as described in Example 17, were co-cultured with either an OPM2 wild-type multiple myeloma cell line or a BCMA knockout (KO) OPM2 cell line for 24 hours at increasing E:T ratios, and cell lysis was assessed. Single-targeting CAR-expressing cell compositions served as controls as follows: an anti-BCMA CAR with a 4-1BB co-stimulatory domain (BCMA-41BB), an anti-GPRC5D CAR with a 4-1BB co-stimulatory domain (GPRC5D-41BB), or an anti-GPRC5D CAR with a CD28 co-stimulatory domain (GPRC5D-CD28). As a negative control, a cell composition was generated in which the cells expressed the same anti-BCMA CAR, but lacking the 4-1BB and CD3zeta signaling domains (BCMA del CAR; SEQ ID NO:318).

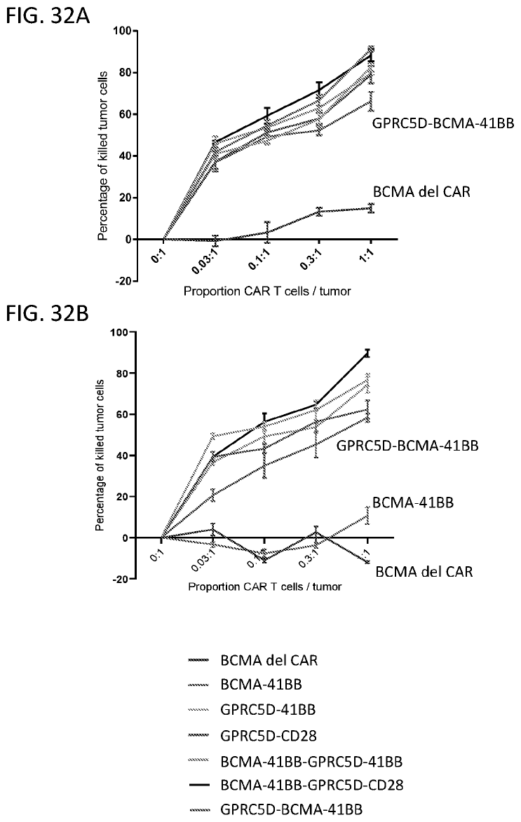

As shown in FIG. 32A, all of the exemplary generated cell compositions were able to effectively lyse the wild-type OPM2 target cells, except cells expressing the BCMA del CAR. As shown in FIG. 32B, all of the exemplary generated cell compositions were able to lyse the BCMA KO OPM2 target cells, except the cells expressing the BCMA del CAR and the cells expressing the single-targeting BCMA-41BB CAR, consistent with selectivity of the anti-BCMA CAR for its target antigen. Results are given as mean±SEM and are shown for a representative experiment that was run in triplicate.

B. Cytokine Secretion of T Cell Compositions

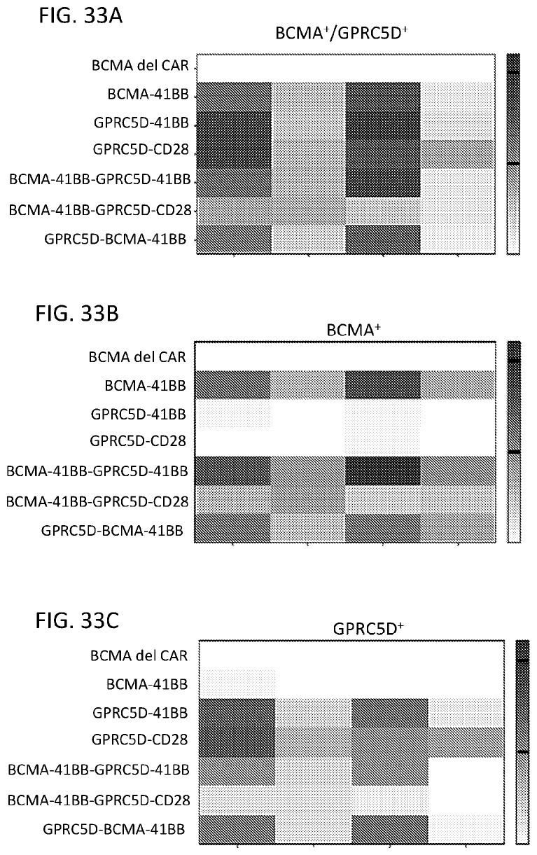

The exemplary generated cell compositions were co-cultured at a 1:1 ratio for 24 hours with target cells expressing both BCMA and GPRC5D (FIG. 33A), only BCMA (FIG. 33B), or only GPRC5D (FIG. 33C). Supernatant was collected for analysis of cytokines by Luminex® multiplex assay. The results are shown in FIGS. 33A-C for exemplary cytokines GM-CSF, IFNg, IL-13 and IL-2 (from left to right); concentrations are in pg/mL, with notches on scale representing 50,000 pg/mL and 100,000 pg/mL.

When cultured with target cells expressing both antigens, all of the exemplary generated cell compositions exhibited cytokine secretion, except for the cells expressing the BCMA del CAR. When cultured with target cells expressing only BCMA, the cell compositions expressing only a single-targeting anti-GRPC5D CAR (GPRC5D-41BB and GPRC5D-CD28) did not secrete the assessed cytokines at detectable levels. Similarly, when cultured with target cells expressing only GPRC5D, the cell composition expressing only a single anti-BCMA CAR (BCMA-41BB) did not secrete the assessed cytokines at detectable levels. These results are generally consistent with a finding that T cells engineered to express the exemplary generated CAR constructs are capable of antigen-specific cytokine secretion.

### Example 19: In Vivo Assessment of Bicistronic Anti-BCMA and Anti-GPRC5D Chimeric Antigen Receptors

Murine multiple myeloma models were used to assess the in vivo effects of T cell compositions containing cells engineered to express the exemplary generated single- and dual-antigen targeting CAR constructs described in Examples 17 and 18. Studies were carried out in an OPM2 murine xenograft model. The OPM2 cell line was engineered to express firefly luciferase (OPM2-ffLuc) or membrane-tethered cyprinda luciferase (OPM2 Cyprinda Luc). NOD scid gamma (NSG™) mice were injected on day 0 via tail vein with 2×10⁶ OPM2-ffLuc cells, which were allowed to engraft and expand for 14 days.

A. Effect of Dual-Targeting CARs in an OPM2 Murine Xenograft Model

Primary human T cell compositions containing (1) cells expressing only the anti-BCMA del CAR; BCMA del CAR, (2) cells expressing only the anti-BCMA CAR incorporating a 4-1BB co-stimulatory domain; BCMA-41BB, (3) cells expressing the “single stalk” construct targeting BCMA and GPRC5D; GPRC5D-BCMA-41BB; or (4) cells expressing a bicistronic construct encoding both an anti-BCMA CAR and an anti-GPRC5D CAR separated by a bicistronic element, in either the BCMA-41BB-GPRC5D-41BB format or the BCMA-41BB-GPRC5D-CD28 format, were tested in an OPM2 murine xenograft model.

On day 14 after engraftment of OPM2-ffLuc cells, mice were treated with a tail vein injection of 3×10⁶ T cells expressing one of the following CAR constructs: (1) BCMA del CAR; (2) BCMA-41BB CAR; (3) BCMA-41BB-GPRC5D-41BB CAR; (4) BCMA-41BB-GPRC5D-CD28 CAR; or (5) GPRC5D-BCMA-41BB. The BCMA del CAR and single-targeting CAR served as controls.

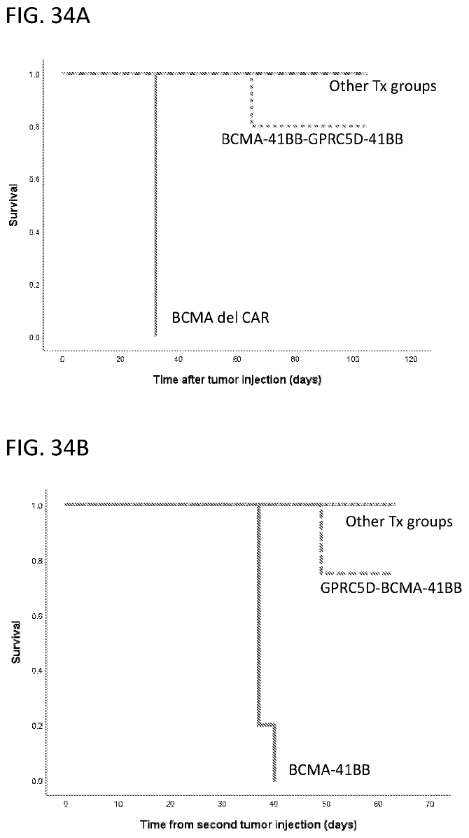

FIG. 34A shows the survival curve of the treated mice. The median overall survival (mOS) of mice treated with the BCMA del CAR (negative control) was 32 days, whereas the mOS of the other groups was not reached when monitored to day 105 (p<0.05).

In a related experiment, a BCMA knockout (KO) OPM2 cell line was generated, substantially as described in previous examples. Surviving mice from FIG. 34A were re-challenged with a second injection of 2×10⁶ BCMA KO OPM2 cells. As shown in FIG. 34B, the mOS of mice treated with cells expressing the single-targeting BCMA-41BB CAR was 37 days after re-challenge, whereas the mOS of mice treated with any of the dual-targeting CAR constructs was not reached (p<0.05).

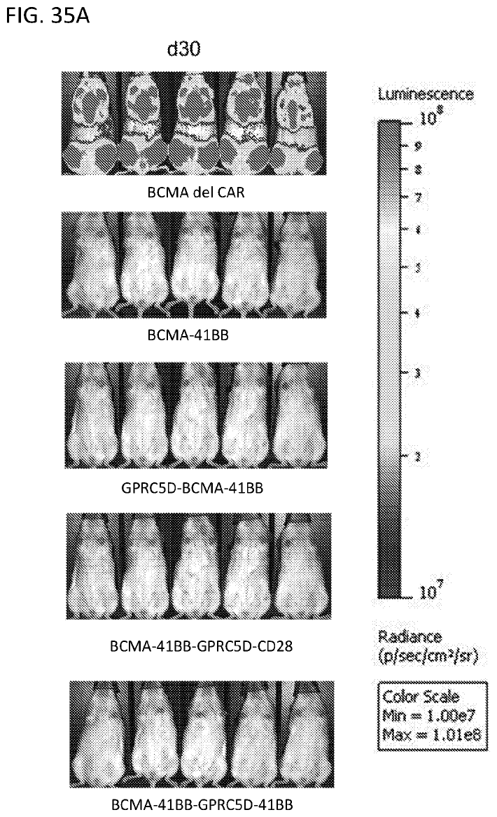

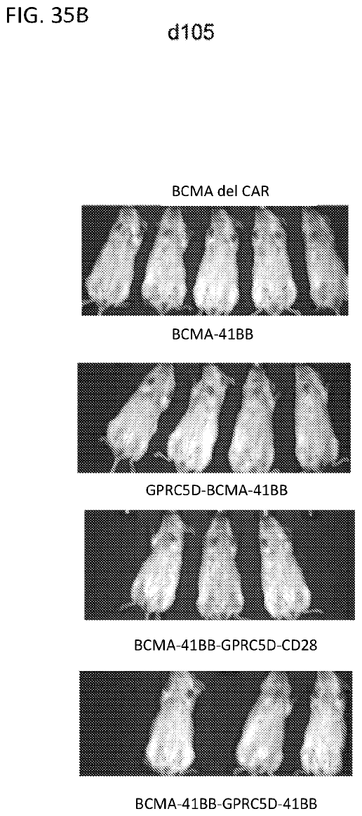

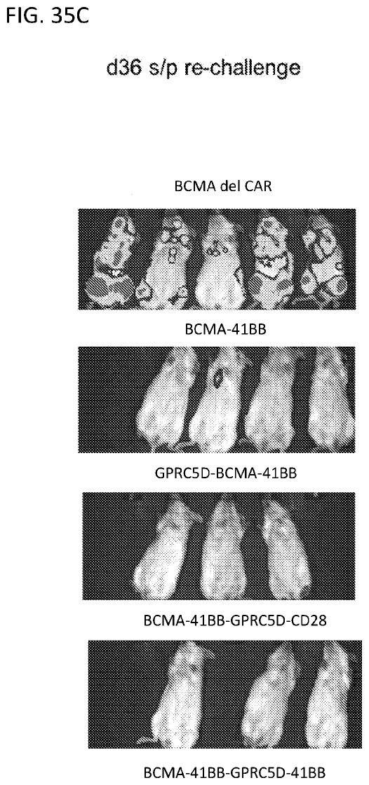

The tumor burden of treated mice was assessed by bioluminescence imaging (BLI) on days 30 and 105 after the original injection of OPM2 cells, and on day 36 after re-challenge with the BCMA KO OPM2 cells (FIGS. 35A-C). As shown in FIG. 35A and FIG. 35B, only mice treated with the BCMA del CAR exhibited tumor burden prior to the re-challenge. As shown in FIG. 35C, mice treated with cells expressing a single-targeting BCMA-41BB CAR exhibited substantial tumor burden following re-challenge with BCMA KO OPM2 cells, as compared to the mice treated with cells expressing dual-targeting CAR constructs.

These results are consistent with a finding that a single-targeting anti-BCMA CAR is less effective in controlling tumor cells not expressing BCMA, as compared to approaches targeting both BCMA and GPRC5D.

B. Comparison of CAR Co-Stimulatory Signaling Domains in a Murine Model of Multiple Myeloma

Following engraftment and expansion of OPM2-ffLuc tumor cells, mice were treated with 5×10⁵ T cells expressing one of the following single- or dual-targeting constructs: (1) BCMA del CAR; (2) BCMA-41BB; (3) GPRC5D-41BB; (4) GPRC5D-CD28; (5) GPRC5D-BCMA-41BB; (6) BCMA-41BB-GPRC5D-41BB; or (7) BCMA-41BB-GPRC5D-CD28. The BCMA del CAR and single-targeting CARs served as controls.

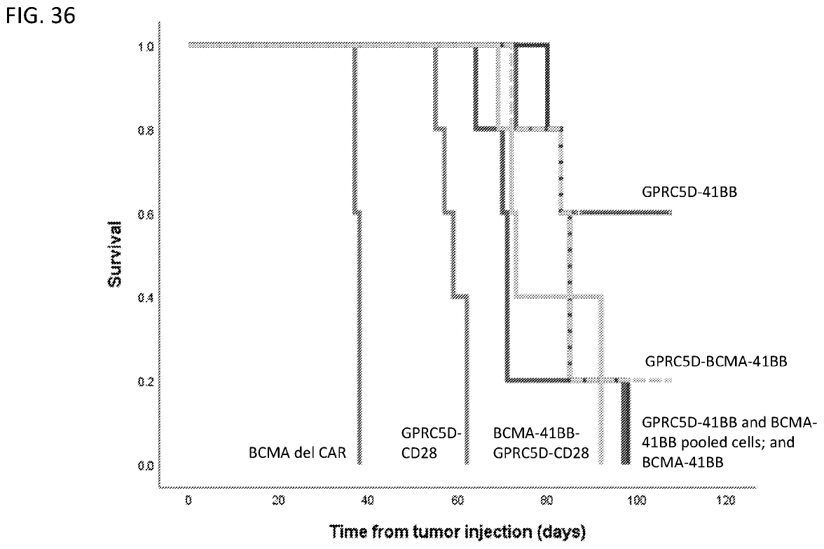

Survival of the mice is shown in FIG. 36. The mOS of mice treated with the BCMA del CAR (negative control) was 38 days. By contrast, the mOS of mice in all other treatment groups was significantly increased beyond 38 days (p<0.05). Treatment with cells expressing the single-targeting anti-GPRC5D CAR incorporating a CD28 co-stimulatory domain was associated with shorter mOS, as compared to treatment with cells expressing the other constructs (p<0.05).

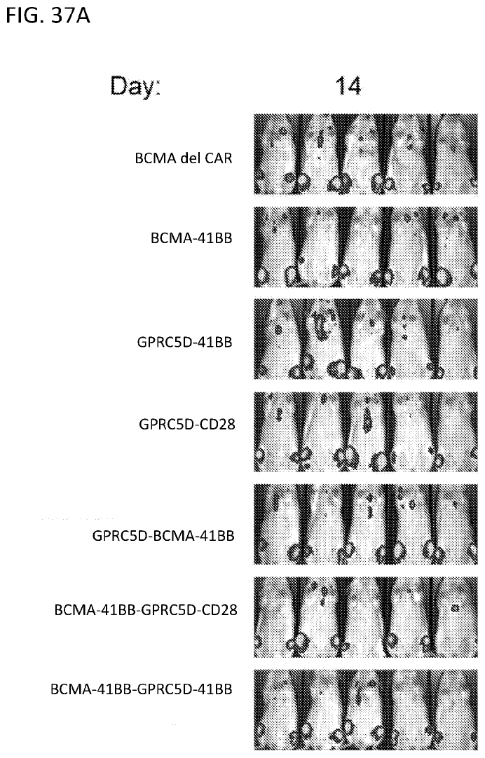

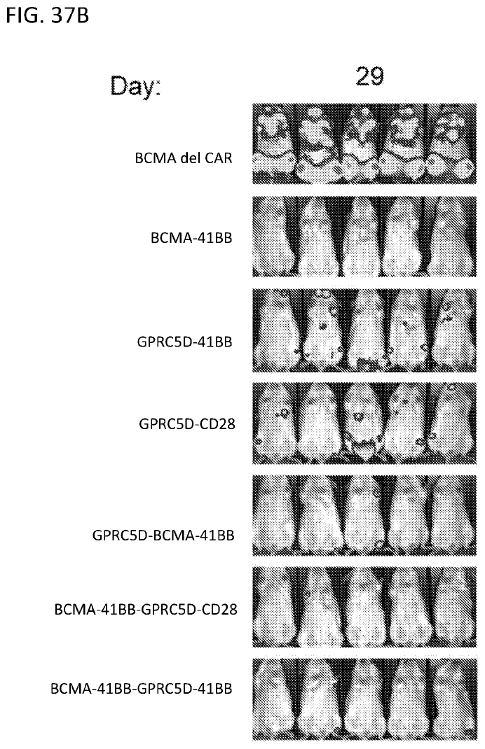

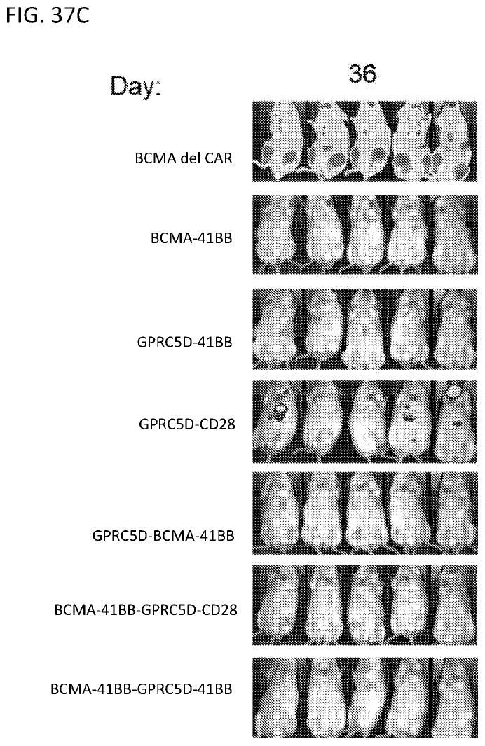

Tumor burden was assessed via bioluminescence imaging (BLI) at 14 days (FIG. 37A), 29 days (FIG. 37B), and 36 days (FIG. 37C) after injection of OPM2 cells. As shown in FIG. 37A, all groups of mice demonstrated substantial tumor burden at the time of treatment with CAR T cell compositions. FIG. 37B and FIG. 37C show that on day 29 or day 36, respectively, after injection of OPM2 cells, mice treated with cells expressing the BCMA del CAR (negative control) exhibited substantial tumor burden. In addition, mice treated with cells expressing the single-targeting GPRC5D-CD28 CAR demonstrated minimal tumor burden. By contrast, all other treated groups had reduced tumor burden on day 29 and did not exhibit appreciable tumor burden on day 36.

C. Comparison of CAR Co-Stimulatory Signaling Domains in an Murine Model of Multiple Myeloma Antigen Escape

To understand the effects of different co-stimulatory domains in a murine multiple myeloma antigen escape model, NSG mice were injected on day 0 via tail vein with 2×10⁶ OPM2 cells engineered to express membrane-tethered cyprinda luciferase (OPM2 Cyprinda Luc), spiked with 5-10% BCMA KO OPM2 cells engineered to express firefly luciferase (Firefly Luc). The different luciferases used allowed for differential imaging of wild-type OPM2 cells and BCMA KO OPM2 cells. Tumor cells were allowed to engraft and expand for 14 days.

On day 14, mice were treated with 5×10⁵T cells engineered to express one of the following constructs: (1) BCMA del CAR; (2) BCMA-41BB; (3) BCMA-41BB and GPRC5D-41BB pooled cells; (4) BCMA-41BB and GPRC5D-CD28 pooled cells; (5) BCMA-41BB-GPRC5D-41BB; (6) BCMA-41BB-GPRC5D-CD28; or (7) GPRC5D-BCMA-41BB. The BCMA del CAR and single-targeting CARs served as controls.

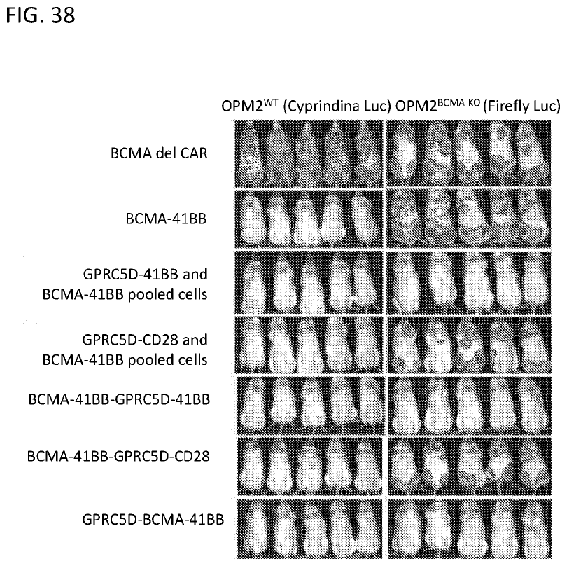

Tumor burden was assessed in mice by BLI on day 28. As shown in FIG. 38, treatment with CAR T cells eradicated OPM2 wild-type cells in all groups. By contrast, CAR T cells expressing an anti-GPRC5D CAR incorporating a CD28 co-stimulatory domain, whether included in a pooled T cell composition or in a bicistronic construct, failed to eradicate BCMA KO OPM2 cells.

In a similar experiment, NSG mice were injected intravenously on day 0 with the mixed composition of OPM2 wild-type and BCMA KO OPM2 cells, to establish a bone marrow-tropic multiple myeloma xenograft model. The cells were allowed to engraft and expand for 14 days, and then mice were treated with a single intravenous injection of 2.5×10⁵ T cells expressing one of the following constructs: (1) BCMA del CAR; (2) BCMA-41BB and GPRC5D-41BB pooled cells; (3) BCMA-41BB-GPRC5D-41BB; and (4) GPRC5D-BCMA-41BB. Mice treated with cells expressing the BCMA del CAR served as a negative control. In this experiment, all of the CAR constructs except the negative control incorporated a 4-1BB co-stimulatory domain

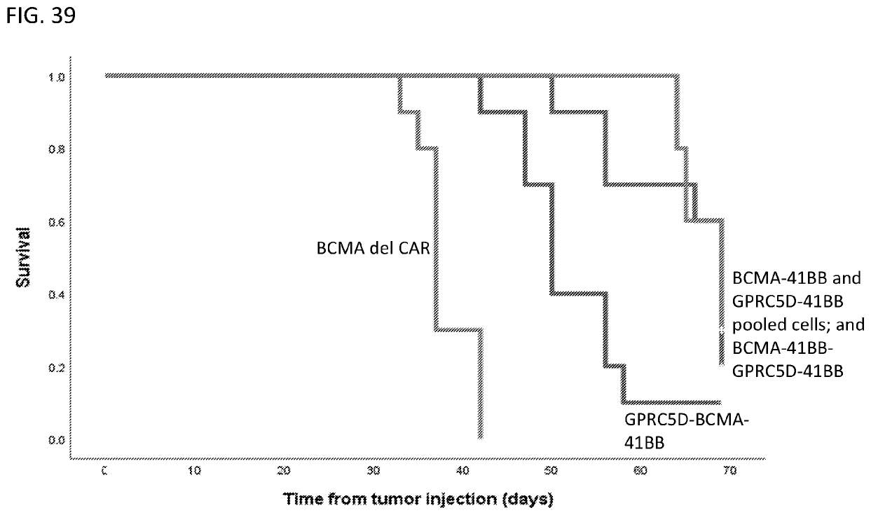

The survival of mice treated with the indicated cell compositions 14 days after OPM2 injection is shown in FIG. 39. Compared to mice treated with cells expressing the BCMA del CAR, all other treatment groups showed significant increases in survival (p<0.001 for all CARs vs. control). Notably, mice treated with cells expressing the single stalk GPRC5D-BCMA-41BB CAR exhibited significantly shorter survival when compared to mice treated with (1) cells expressing the BCMA-41BB-GPRC5D-41BB construct or (2) cells expressing the GPRC5D-41BB CAR and cells expressing the BCMA-41BB CAR, pooled at a 1:1 ratio (p<0.05 for single stalk construct compared to either of the other two dual-targeting approaches).

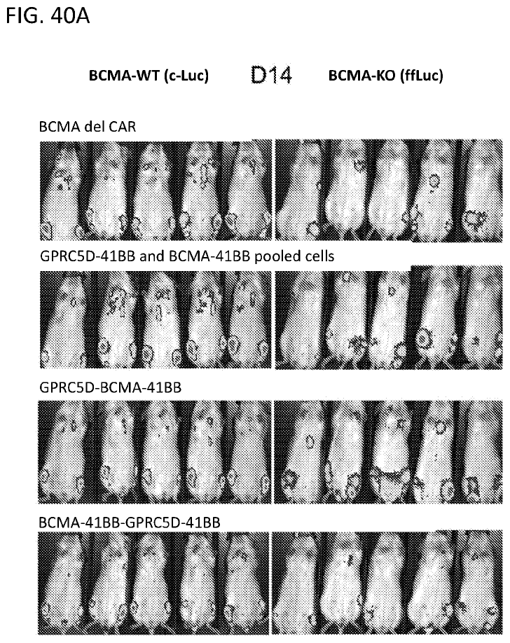

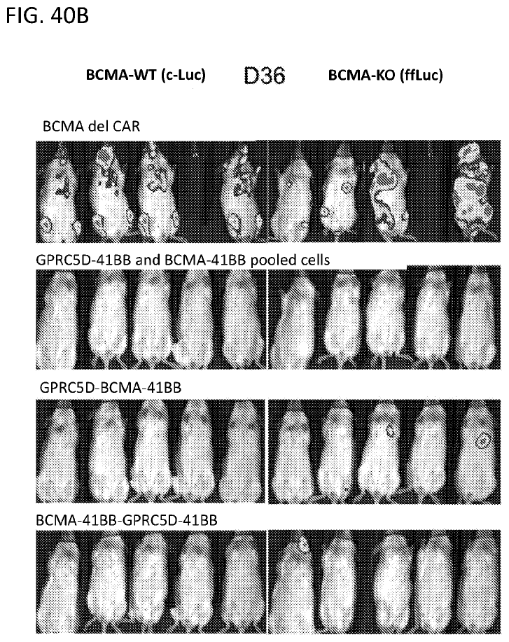

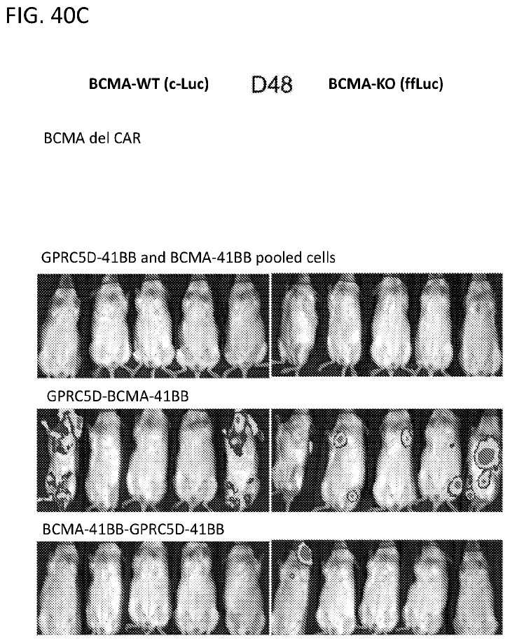

Tumor burden of the bone marrow-tropic model was assessed by BLI 14 days (FIG. 40A), 36 days (FIG. 40B), and 48 days (FIG. 40C) after injection of tumor cells. All treatment groups exhibited tumor burden at the time of CAR T cell treatment as shown in FIG. 40A. As shown in FIG. 40B, all treatments were able to effectively control OPM2 wild-type cells at day 36. However, as shown in FIG. 40C, by day 48 the GPRC5D-4-1BB/BCMA-41BB CAR pooled cell composition and the BCMA-41BB-GPRC5D-41BB bicistronic construct cell composition was more effective than a composition containing cells expressing the single stalk CAR construct (GPRC5D-BCMA-41BB).

### Example 20: Generation of Additional Bicistronic Polynucleotides Encoding Anti-GPRC5D and Anti-BCMA Chimeric Antigen Receptors

Additional bicistronic polynucleotides encoding both an anti-GPRC5D CAR and an anti-BCMA CAR were generated.

The exemplary anti-GPRC5D CAR contained an scFv antigen-binding domain able to bind to GPRC5D (e.g. GPRC5D-203 V_{L}/V_{H} scFv (SEQ ID NO: 8); an immunoglobulin hinge-CH2-CH3 set forth in SEQ ID NO: 17, encoded by the sequence set forth in SEQ ID NO:73; a human CD28-derived transmembrane domain (e.g. SEQ ID NO: 18); a human 4-1BB-derived intracellular signaling domain (e.g. SEQ ID NO: 19); and a human CD3zeta-derived intracellular signaling domain (e.g. SEQ ID NO: 20).

The exemplary anti-BCMA CAR contained an scFv antigen-binding domain able to bind cells expressing BCMA (e.g. BCMA-55, V_{L}/V_{H} scFv (SEQ ID NO: 241); an immunoglobulin hinge-CH2-CH3 set forth in SEQ ID NO: 17, encoded by the sequence set forth in SEQ ID NO:73; a human CD28-derived transmembrane domain (e.g. SEQ ID NO: 18); a human 4-1BB-derived intracellular signaling domain (e.g. SEQ ID NO: 19); and a human CD3zeta-derived intracellular signaling domain (e.g. SEQ ID NO: 20).

The nucleotide sequences encoding each of the CARs were separately codon optimized prior to their combination into a single bicistronic polynucleotide construct.

The bicistronic polynucleotides were generated in two formats. The first format was generated with the nucleotide sequence encoding the anti-GPRC5D CAR located toward the 5′ end of the polynucleotide (N-terminal of a polypeptide) relative to the nucleotide sequence encoding the anti-BCMA CAR (“αGPRC5D-αBCMA”; SEQ ID NO:303 encoding SEQ ID NO:301). The second format was generated with the nucleotide sequence encoding the anti-BCMA CAR located toward the 5′ end of the polynucleotide (N-terminal of a polypeptide) relative to the nucleotide sequence encoding the anti-GPRC5D CAR (“αBCMA-αGPRC5D”; SEQ ID NO:300 encoding SEQ ID NO:298). The nucleotide sequences encoding the anti-GPRC5D CAR and the anti-BCMA CAR were separated by a nucleotide sequence encoding a downstream ribosome skip element (such as a self-cleaving T2A peptide; SEQ ID NO: 44 or 45 encoding SEQ ID NO:37).

The polynucleotide constructs were cloned into a lentiviral expression vector for transduction of T cells. Jurkat cells were stably transduced with the lentiviral vector containing the polynucleotide bicistronic constructs containing either the αGPRC5D-αBCMA CAR format or the αBCMA-αGPRC5D CAR format. As a control, Jurkat cells were also transduced with constructs encoding the single antigen-targeting anti-GRPC5D CAR or anti-BCMA CAR. Transduction was carried out with 5×10⁵ cells/well and expression of the CAR was determined on day 3. Expression of the CARs was assessed by flow cytometry using a binding reagent specific to the antigen-binding domain of each CAR.

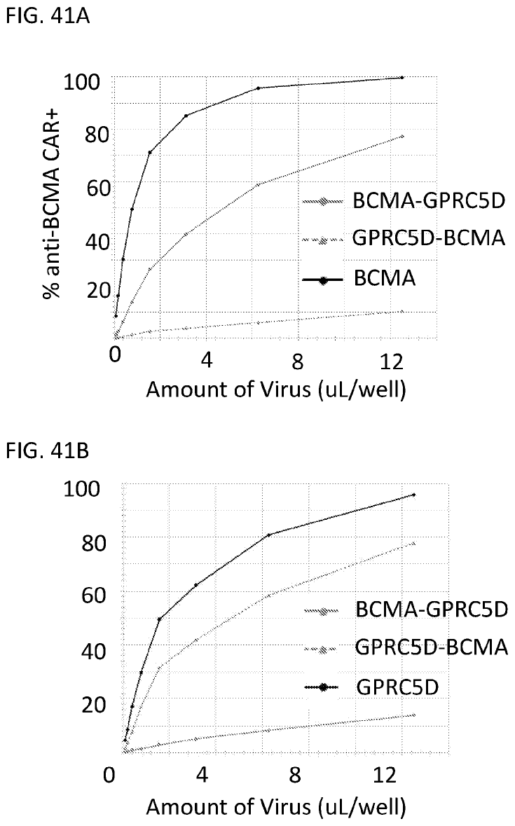

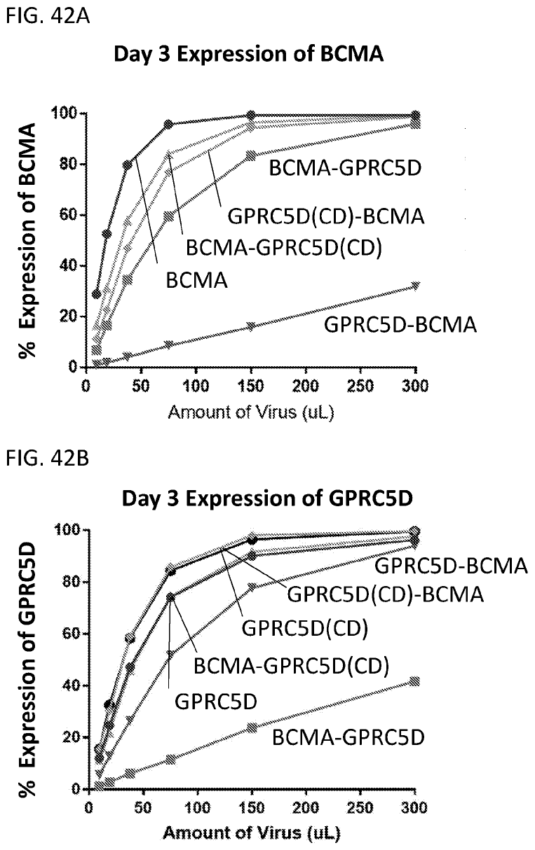

Flow cytometric analysis showed that expression of the CAR encoded by the 3′-located nucleotide sequence (trailing CAR) was significantly decreased in both bicistronic formats compared to the CAR encoded by the 5′-located nucleotide sequence (leading CAR) or compared to expression of a construct encoding only a single CAR. In particular, FIG. 41A depicts results for percent expression of anti-BCMA CAR among assessed formats, and FIG. 41B shows expression of the anti-GPRC5D CAR among assessed formats. PCR analysis of the genomic DNA of transduced Jurkat cells indicated that the 3′-located CAR-encoding nucleotide sequence was lost through recombination (data not shown).

### Example 21: Codon Divergence of Bicistronic Polynucleotides Encoding Anti-GPRC5D and Anti-BCMA Chimeric Antigen Receptors

To reduce possible recombination and loss of the 3′-located CAR-encoding nucleotide sequence, the nucleic acid sequence encoding one of the CARs was codon diverged. In this example, the nucleic acid sequence encoding the anti-GPRC5D CAR was codon diverged compared to the sequence described in Example 20.

Specifically, the portions of the nucleotide sequence encoding the scFv, immunoglobulin spacer, the CD28 transmembrane domain, the 4-1BB endodomain, and the CD3zeta endodomain were codon diverged, such that the nucleotides encoding each of these components differed at the nucleotide level compared to the nucleotide sequence encoding the same component in the anti-BCMA CAR, while still encoding the identical amino acid sequences. The nucleotide sequence was codon diverged such that there were no more than 10 consecutive (or contiguous) base pairs of sequence homology between the nucleotide sequence encoding the anti-GPRC5D CAR and the nucleotide sequence encoding the anti-BCMA CAR of the bicistronic construct. Following codon divergence of the anti-GPRC5D CAR, the polynucleotides were analyzed for splice sites (e.g. NNSPLICE version 0.9 online splice-site prediction tool; fruitfly.org, Berkeley Drosophila Genome Project, Berkeley, Calif.). Splice donor sites and splice acceptor sites were evaluated independently. Identified splice donor and splice acceptor sites with a splice site score of >0.7 (>70% probability of a splice event), e.g., in promoter region and long spacer region), were modified by silent mutation to reduce the splice site score to less than 0.7.

Lentiviral vectors containing the polynucleotide bicistronic constructs were used to transduce Jurkat T cells and cell surface expression of the CARs were monitored by flow cytometry using a binding reagent specific to each CAR antigen-binding domain.

As shown in FIGS. 42A and 42B, codon divergence of the bicistronic polynucleotide construct substantially improved expression of the CAR encoded by the 3′-located nucleotide sequence compared to expression of the same CAR encoded by the original bicistronic polynucleotide. For example, as shown in FIG. 42A, the expression of the anti-BCMA CAR was relatively low in cells that had been transduced with the original αGPRC5D-αBCMA bicistronic construct, but this expression was substantially improved in cells that had been transduced with the respective codon diverged (CD) construct. Likewise, as shown in FIG. 42B, the expression of the anti-GPRC5D CAR was relatively low in cells that had been transduced with the original αBCMA-αGPRC5D bicistronic construct, but this expression was substantially improved in cells that had been transduced with the respective codon diverged (CD) construct.

### Example 22: In Vitro Assessment of GPRC5D/BCMA Dual-Targeting CAR-Engineered T Cell Compositions

T cell compositions, either Jurkat Nur77-RFP reporter or primary T cells, containing (1) cells engineered to express only the anti-GPRC5D CAR (αGPRC5D), (2) cells engineered to express only the anti-BCMA CAR (αBCMA), or (3) dual-targeting cells expressing both CARs by transduction of codon-diverged bicistronic constructs as described in Example 5, in the αBCMA-αGPRC5D or αGPRC5D-αBCMA formats were each assessed for antigen-specific activity upon co-culture with antigen-expressing cells. Either a Nur77-RFP Jurkat T cell line or primary T cells isolated by immunoaffinity-based enrichment from leukapheresis samples from human donor subjects were transduced with the lentiviral vector encoding the single CAR constructs or containing the bicistronic construct encoding the anti-BCMA CAR and anti-GPRC5D CAR (dual-targeting cells). After transduction, and in some cases expansion, T cells were stained with an antibody specific for each CAR and analyzed by flow cytometry, confirming transduction of cells and expression of the CARs.

In some experiments, a pooled T cell composition containing cells separately expressing the anti-BCMA CAR and anti-GPRC5D CAR, as described in Example 13, was assessed (“αGPRC5D αBCMA and pooled cells”).

A. Antigen-Dependent Activity in the Presence of Multiple Myeloma Cell Lines

The engineered Jurkat cells were co-cultured at an E:T ratio of 1:1 for 20 hours with multiple myeloma cell lines expressing varying amounts of BCMA and GPRC5D. The multiple myeloma cell lines included KMS12BM, MM.1S, NCI-H929, OPM-2, and RPMI8226 (see Example 16 describing levels of expression of GPRC5D and BCMA on the cell lines). The chronic myelogenous leukemia (CML) cell line K562, as well as a K562 cell line engineered to express GPRC5D, were included as negative and positive controls, respectively. Antigen-specific stimulation of the CAR was assessed by measuring changes in RFP expression by flow cytometry.

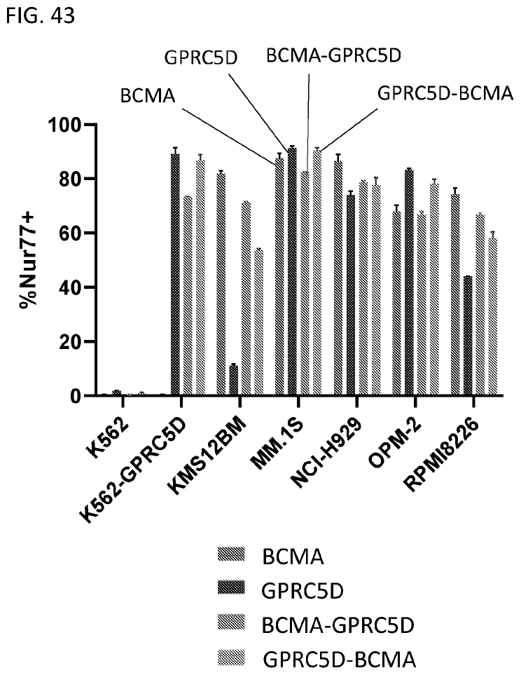

As shown in FIG. 43, Jurkat Nur77/RFP reporter cells expressing the αBCMA, αGPRC5D, αBCMA-αGPRC5D, or αGPRC5D-αBCMA CARs were stimulated by co-culture with the various multiple myeloma cell lines as evidenced by the percentage of cells positive for RFP signal (% Nur77). As shown, cells expressing only the αGPRC5D CAR were less responsive to certain target cells such as the KMS12BM cell line but reporter activity was increased in cell compositions expressing the dual-targeting CARs.

B. Cytokine Secretion in the Presence of Multiple Myeloma Cell Lines

Primary human T cell compositions containing (1) T cells engineered to express only the anti-GPRC5D CAR (αGPRC5D), (2) T cells engineered to express only the anti-BCMA CAR (αBCMA), (3) T cells containing dual-targeting cells expressing both CARs by transduction of bicistronic constructs in the αBCMA-αGPRC5D or αGPRC5D-αBCMA formats, or (4) pooled T cells separately engineered to express either the anti-BCMA CAR or the anti-GPRC5D CAR pooled at 1:1 ratio of anti-BCMA CAR-expressing cells to anti-GPRC5D CAR-expressing cells (αGPRC5D and αBCMA pooled cells) were each assessed for antigen-specific activity. As a control, primary human T cell compositions were mock transduced. T cells were co-cultured at an E:T ratio of 1:1 for 24 hours with the KMS12BM, MM.15, NCI-H929, OPM2, and RPMI8226 multiple myeloma cell lines, and supernatants were collected for analysis of cytokines by Luminex® multiplex assay.

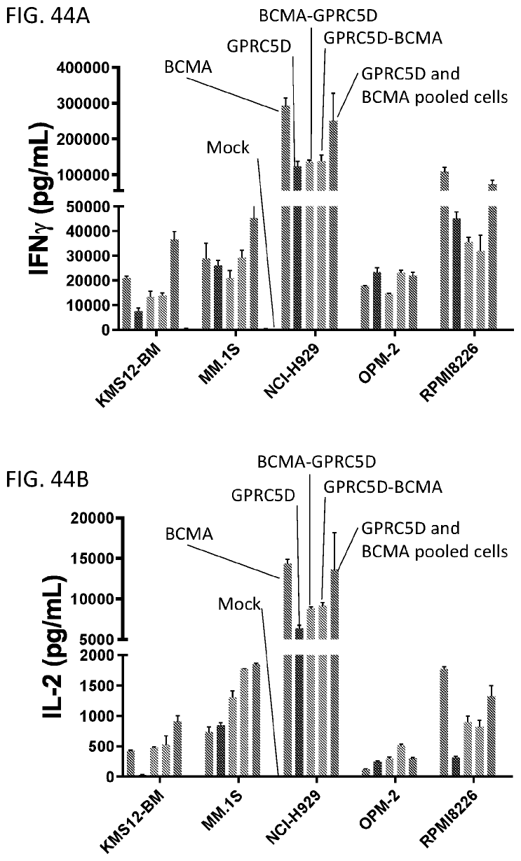

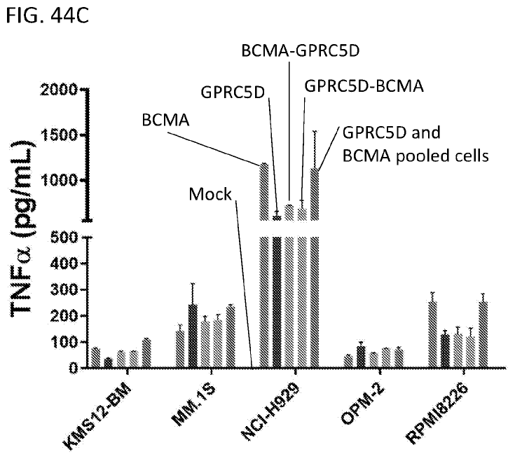

Secretion levels of IFNγ, IL-2, TNFα were assessed after co-culture of the primary human T cell compositions with the various multiple myeloma cell lines, as shown in FIGS. 44A, 44B, and 44C, respectively. Cytokine secretion was observed for all of the primary human T cell compositions tested, except the mock-transduced T cell composition. For certain cell lines, cytokine secretion was increased by cell compositions containing dual-targeting the αBCMA-αGPRC5D or αGPRC5D-αBCMA formats or αGPRC5D and αBCMA pooled cells, compared to cell compositions engineered to express only αGPRC5D or only αBCMA CARs.

D. Antigen-Dependent Activity in the Presence of BCMA or GPRC5D KO Cells

To understand the antigen-dependent activity of the anti-BCMA and anti-GPRC5D CARs in the context of antigen loss, an antigen loss model was created by knocking out either GPRC5D or BCMA from OPM2 cells. GPRC5D was knocked out of OPM2 cells as described in Example 5. BMCA was knocked out of OPM2 cells as described in Example 12.

Flow cytometric analysis confirmed a lack of GPRC5D expression in the OPM2 GPRC5D KO cells and a lack of BCMA expression in the OPM2 BCMA KO cells. OPM2 wild-type (OPM2 WT) cells served as a control, showing expression of both GPRC5D and BCMA (see, e.g., FIG. 22A).

Jurkat Nur77-RFP reporter cell compositions containing cells engineered to express only the anti-GPRC5D CAR (αGPRC5D) or only the anti-BCMA CAR (αBCMA), or containing dual-targeting cells expressing both CARs by transduction of bicistronic constructs in the αBCMA-αGPRC5D or αGPRC5D-αBCMA formats were assessed for antigen-specific activity. The various Jurkat Nur77-RFP report cell compositions were co-cultured with OPM2 WT, OPM2 GPRC5D KO, or OPM2 BCMA KO cells at a 1:1 ratio for 20 hours. Antigen-specific stimulation of the CARs expressed by the cell compositions was assessed by measuring changes in RFP expression by flow cytometry.

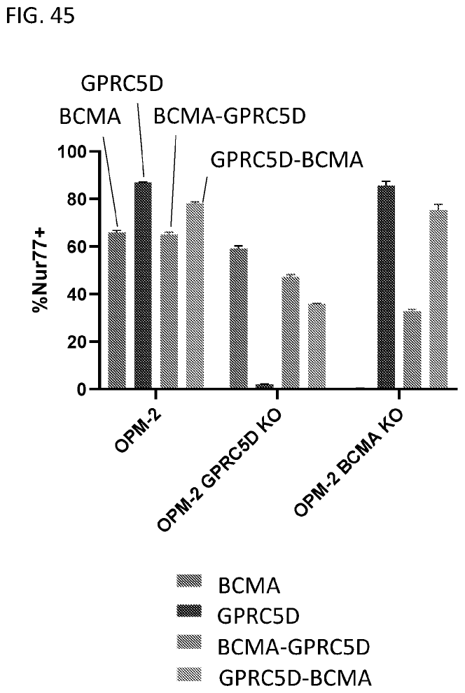

As shown in FIG. 45, activation of non-pooled Jurkat Nur77-RFP reporter cells expressing either only the GPRC5D-targeting CAR (αGPRC5D) or only the BCMA-targeting CAR (αBCMA) was not observed when they were co-cultured with OPM2 cells in which GPRC5D or BCMA was knocked out, respectively. By contrast, activation was maintained in Jurkat Nur77-RFP reporter dual-targeting cell compositions containing cells expressing the αBCMA-αGPRC5D or αGPRC5D-αBCMA CARs when BCMA or GPRC5D was knocked out of OPM2 cells. These results demonstrate that the dual-targeting engineered T cell compositions containing αBCMA-αGPRC5D and αGPRC5D-αBCMA CAR formats are capable of mediating activation even in the absence of one of the targeted antigens.

E. Cytokine Secretion in the Presence of BCMA or GPRC5D KO Cells

To further assess the effects of antigen loss, OPM2 WT, OPM2 GPRC5D KO, or OPM2 BCMA KO cells were cultured at a 1:1 ratio for 24 hours with primary human T cell compositions, and cytokine secretion by the T cells was assessed. The primary human T cell compositions contained (1) cells engineered to express only the anti-GPRC5D CAR (αGPRC5D), (2) only the anti-BCMA CAR (αBCMA), (3) dual-targeting cells expressing both CARs by transduction of bicistronic constructs in the αBCMA-αGPRC5D or αGPRC5D-αBCMA formats, or (4) pooled cells separately engineered to express either the anti-BCMA CAR or the anti-GPRC5D CAR pooled at 1:1 ratio of anti-BCMA CAR-expressing cells to anti-GPRC5D CAR-expressing cells CAR (αGPRC5D and αBCMA pooled cells). Mock transduced T cell compositions served as a control.

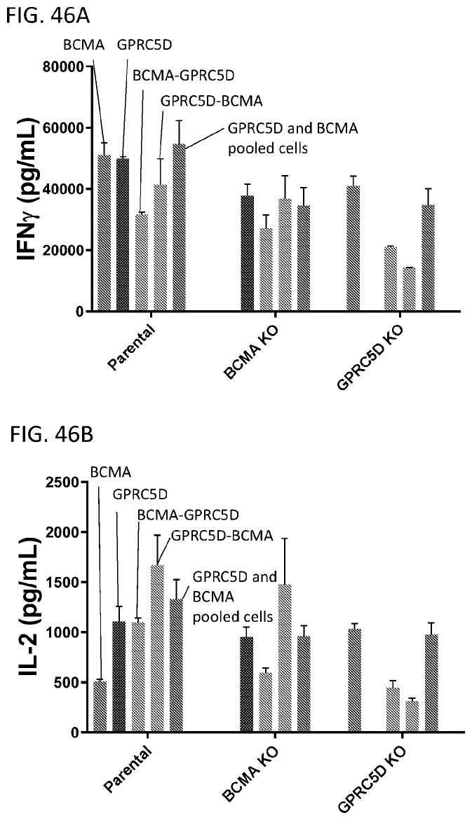

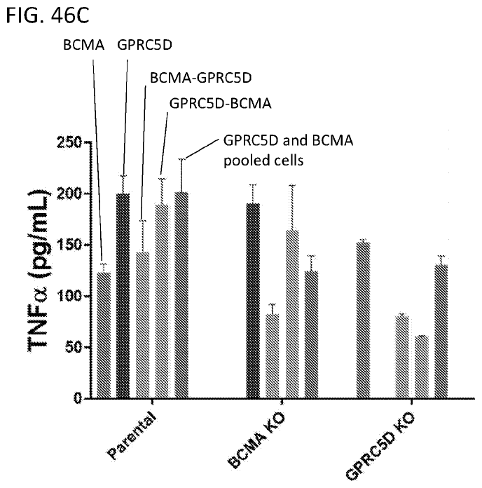

Following co-culture with the OPM2 cells, supernatants were collected for analysis of cytokines by Luminex® multiplex assay. Secretion levels of IFNγ, IL-2, TNFα were assessed, as shown in FIGS. 46A, 46B, and 46C, respectively. Cytokine secretion by T cells expressing either only the anti-GPRC5D CAR (αGPRC5D) or only the anti-BCMA CAR (αBCMA) was not observed when they were co-cultured with OPM2 cells in which GPRC5D or BCMA was knocked out, respectively. However, cytokine secretion was maintained in T cells in compositions containing dual-targeting cells expressing the αBCMA-αGPRC5D or αGPRC5D-αBCMA CARs, and in the αGPRC5D and αBCMA pooled cell compositions when either GPRC5D or BCMA was knocked out of OPM2 cells. These results indicate that dual-targeting cells, in which either cells are co-transduced with the αBCMA-αGPRC5D or αGPRC5D-αBCMA CAR formats or include αGPRC5D and αBCMA pooled cells, are capable of secreting cytokines, despite the absence of one of the antigens.

### Example 23: In Vivo Assessment of Dual-Targeting Cell Compositions Containing Cells Expressing GPRC5D- and BCMA-Targeting CARs

An OPM2 murine multiple myeloma model was generated and used to assess the in vivo effects of T cell compositions containing (1) cells engineered to express only the anti-GPRC5D CAR (αGPRC5D), (2) cells engineered to express only the anti-BCMA CAR (αBCMA), (3) dual-targeting cells expressing both CARs by transduction of a bicistronic construct in the αGPRC5D-αBCMA format, or (4) pooled cells separately engineered to express either the anti-BCMA CAR or the anti-GPRC5D CAR pooled at 1:1 ratio of anti-BCMA CAR-expressing cells to anti-GPRC5D CAR-expressing cells (αGPRC5D and αBCMA pooled cells).

A. Assessment of T Cell Compositions Expressing Anti-BCMA and Anti-GPRC5D CARs in an OPM2 Murine Xenograft Model

An experiment was conducted in which NOD scid gamma (NSG™) mice were treated with 3×10⁶ total primary human T cells from compositions containing (1) cells expressing the anti-BCMA CAR (αBCMA), (2) cells expressing the anti-GPRC5D CAR (αGPRC5D), (3) dual-targeting cells generated with a bicistronic construct encoding both CARs in the αGPRC5D-αBCMA format, or (4) pooled cells separately engineered to express either the anti-BCMA CAR or the anti-GPRC5D CAR pooled at 1:1 ratio of anti-BCMA CAR-expressing cells to anti-GPRC5D CAR-expressing cells (αGPRC5D and αBCMA pooled cells). The T cell compositions were administered 14 days after injection with OPM2 cell compositions. In this experiment, mice were injected with either 100% OPM2 WT cells, 95% OPM2 WT cells and 5% OPM2 BCMA KO cells, or 95% OPM2 WT cells and 5% OPM2 GPRC5D KO cells. As a control, mice were also treated with mock transduced primary human T cell compositions.

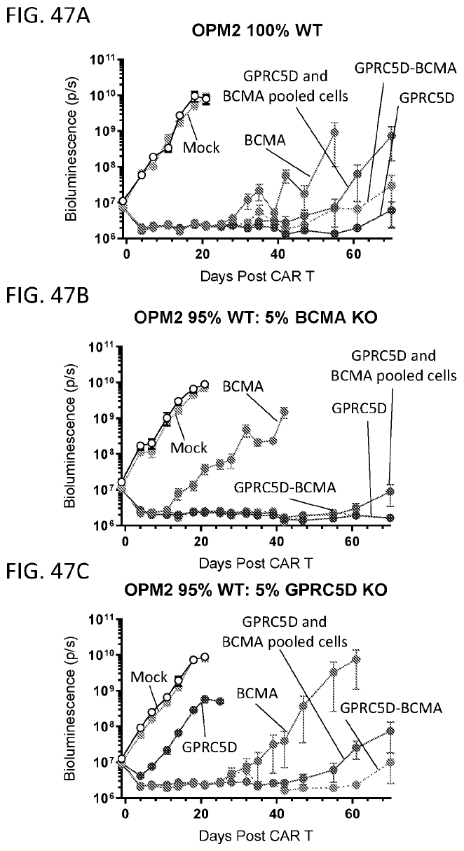

As shown in FIG. 47A, tumor burden as assessed by BLI increased substantially in untreated and mock-treated mice injected that had been administered 100% OPM2 WT cells. Increases in tumor burden were also observed by day 40 in mice treated with cells expressing only the anti-BCMA CAR. By contrast, as shown in FIG. 47B, treatment with a T cell composition containing cells expressing only the anti-BCMA CAR resulted in increasing tumor burden prior to day 20 in mice injected with 5% OPM2 BCMA KO cells. Similarly, mice treated with a T cell composition containing cells expressing only the anti-GPRC5D CAR resulted in increasing tumor burden prior to day 10 in mice injected with 5% OPM2 GPRC5D KO cells (FIG. 47C). Mice treated with the composition containing αGPRC5D and αBCMA pooled cells, as well as mice treated with the composition containing dual-targeting cells expressing CARs from the αGPRC5D-αBCMA bicistronic construct, exhibited relatively minimal increases in tumor burden through day 60. Further, tumor burden in mice treated with 5% OPM2 GPRC5D KO cells was best controlled by treatment with cells expressing the αGPRC5D-αBCMA bicistronic construct.

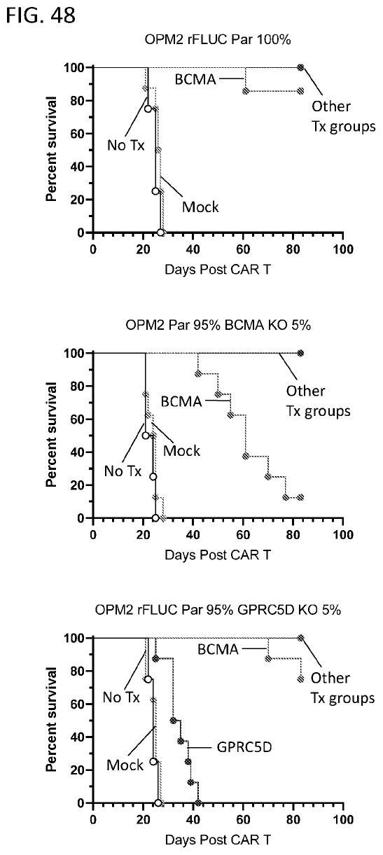

Similar results were observed in this study from analysis of survival of mice past day 80 post-CAR T cell injection (FIG. 48). As shown, survival was decreased in mice that were injected with 5% OPM2 GPRC5D KO cells and treated only with cells expressing the anti-GPRC5D CAR. Survival was also decreased in mice that were injected with 5% OPM2 BCMA KO cells and treated only with cells expressing the anti-BCMA CAR. The other treatment groups exhibited

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | ANNOTATION |
|---|---|---|
| 1 | EVQLVESGGAFVQPGGSLRLSCAASGFTFSSYAMTWVRQAPGKGLEW | GPRC5D-200 |
| | VSTISGRGRSTFYADSVKGRFTISRDNSKNTLYLQMNSLRAEDTAVY | VH/VL |
| | YCARYYHAGAFDLWGQGTLVTVSSGSRGGGGSGGGGSGGGGSLEMAQ | |
| | SVVTQPASVSGSPGQSITISCTGTSSDVGGYNYVSWYQQHPGKAPKL | |
| | MIYDVSKRPSGVSNRFSGSKSGNTASLTISGLQAEDEADYYCSSYTS | |
| | SSTLVFGGGTKLTVL | |
| 2 | QSVVTQPASVSGSPGQSITISCTGTSSDVGGYNYVSWYQQHPGKAPK | GPRC5D-200 |
| | LMIYDVSKRPSGVSNRFSGSKSGNTASLTISGLQAEDEADYYCSSYT | VL/VH |
| | SSSTLVFGGGTKLTVLGSRGGGGSGGGGSGGGGSLEMAEVQLVESGG | |
| | AFVQPGGSLRLSCAASGFTFSSYAMTWVRQAPGKGLEWVSTISGRGR | |
| | STFYADSVKGRFTISRDNSKNTLYLQMNSLRAEDTAVYYCARYYHAG | |
| | AFDLWGQGTLVTVSS | |
| 3 | QMQLVQSGAEVKKPGASVKVSCKASGYTFNRYAITWVRQAPGQGLEW | GPRC5D-201 |
| | MGWISAYNGNSHYAQKLQGRVTMTTDTSTGTAYMELRRLRSDDTAVY | VH/VL |
| | YCARMAYDSWGQGTLVTVSSGSRGGGGSGGGGSGGGGSLEMAQSVLT | |
| | QPASVSGSPGQSLTISCTGTSNDVGAYKYVSWYQQYPGKAPKLILYD | |
| | VFKRPSGVSNRFSGSKSDNTASLTISGLQAEDEADYYCFSLTSSNTY | |
| | VFGTGTKVTVL | |
| 4 | QSVLTQPASVSGSPGQSLTISCTGTSNDVGAYKYVSWYQQYPGKAPK | GPRC5D-201 |
| | LILYDVFKRPSGVSNRFSGSKSDNTASLTISGLQAEDEADYYCFSLT | VL/VH |
| | SSNTYVFGTGTKVTVLGSRGGGGSGGGGSGGGGSLEMAQMQLVQSGA | |
| | EVKKPGASVKVSCKASGYTFNRYAITWVRQAPGQGLEWMGWISAYNG | |
| | NSHYAQKLQGRVTMTTDTSTGTAYMELRRLRSDDTAVYYCARMAYDS | |
| | WGQGTLVTVSS | |
| 5 | EVQLVESGGGLVKPGGSLRLSCAASGFTFSDYYMSWIRQAPGKGLEW | GPRC5D-202 |
| | VSYISSSGSTIYYADSVKGRFTISRDNAKNSLYLQMNSLRAEDTAVY | VH/VL |
| | YCARGYGKAYDQWGQGTLVTVSSGSRGGGGSGGGGSGGGGSLEMAQS | |
| | VLTQPPSASGTPGQRVTISCSGSRSNVGGNYVFWYQQVPGATPKLLI | |
| | YRSNQRPSGVPDRFAGSKSGSSASLAISGLRSEDEADYYCATWDDSL | |
| | SGFVFGTGTKVTVL | |
| 6 | QSVLTQPPSASGTPGQRVTISCSGSRSNVGGNYVFWYQQVPGATPKL | GPRC5D-202 |
| | LIYRSNQRPSGVPDRFAGSKSGSSASLAISGLRSEDEADYYCATWDD | VL/VH |
| | SLSGFVFGTGTKVTVLGSRGGGGSGGGGSGGGGSLEMAEVQLVESGG | |
| | GLVKPGGSLRLSCAASGFTFSDYYMSWIRQAPGKGLEWVSYISSSGS | |
| | TIYYADSVKGRFTISRDNAKNSLYLQMNSLRAEDTAVYYCARGYGKA | |
| | YDQWGQGTLVTVSS | |
| 7 | QVQLVESGGGLVHPGGSLRLSCAASGFTFRSHSMNWVRQAPGKGLEW | GPRC5D-203 |
| | VSSISSDSTYTYYADSVKGRFTISRDNAKNSLYLQMNSLRAEDTAVY | VH/VL |
| | YCARSGGQWKYYDYWGQGTLVTVSSGSRGGGGSGGGGSGGGGSLEMA | |
| | SSELTQDPAVSVALGQTVRITCQGDSLRSYYASWYQQKPGQAPVLVI | |
| | YGKNNRPSGIPDRFSGSSSGNTASLTITGAQAEDEADYYCNSRDSSG | |
| | NPPVVFGGGTKLTVL | |
| 8 | SSELTQDPAVSVALGQTVRITCQGDSLRSYYASWYQQKPGQAPVLVI | GPRC5D-203 |
| | YGKNNRPSGIPDRFSGSSSGNTASLTITGAQAEDEADYYCNSRDSSG | VL/VH |
| | NPPVVFGGGTKLTVLGSRGGGGSGGGGSGGGGSLEMAQVQLVESGGG | |
| | LVHPGGSLRLSCAASGFTFRSHSMNWVRQAPGKGLEWVSSISSDSTY | |
| | TYYADSVKGRFTISRDNAKNSLYLQMNSLRAEDTAVYYCARSGGQWK | |
| | YYDYWGQGTLVTVSS | |
| 9 | EVQLVESGGGLVQPGGSLRLSCAASGFTFSNYAMSWVRQAPGKGLEW | GPRC5D-204 |
| | VSAISGSGNTYYADSVKGRFTISRDNSKNTLYLQMNSLRAEDTAVYY | VH/VL |
| | CARGSVRYTDIWGQGTLVTVSSGSRGGGGSGGGGSGGGGSLEMANFM | |
| | LTQPHSVSESPGKTVSISCTRTSGAIAGAYVQWFQQRPGSAPTTVIY | |
| | DDNKRPSGVPDRFSGSIDKSSNSASLTISGLKTEDEADYYCQSYDYD | |
| | SSNVLFGGGTKLTVL | |
| 10 | NFMLTQPHSVSESPGKTVSISCTRTSGAIAGAYVQWFQQRPGSAPTT | GPRC5D-204 |
| | VIYDDNKRPSGVPDRFSGSIDKSSNSASLTISGLKTEDEADYYCQSY | VL/VH |
| | DYDSSNVLFGGGTKLTVLGSRGGGGSGGGGSGGGGSLEMAEVQLVES | |
| | GGGLVQPGGSLRLSCAASGFTFSNYAMSWVRQAPGKGLEWVSAISGS | |
| | GNTYYADSVKGRFTISRDNSKNTLYLQMNSLRAEDTAVYYCARGSVR | |
| | YTDIWGQGTLVTVSS | |
| 11 | EVQLVESGGGLIQPGGSLRLSCAASGFTFSNYAMNWVRQAPGKGLEW | GPRC5D-205 |
| | VSTINGRGSSTIYADSVKGRFTISRDNSKNTLYLQMNSLRAEDTATY | VH/VL |
| | YCARYISRGLGDSWGQGTLVTVSSGSRGGGGSGGGGSGGGGSLEMAQ | |
| | SVVTQPPSMSAAPGQQVTISCSGGNSNIERNYVSWYLQLPGTAPKLV | |
| | IFDNDRRPSGIPDRFSGSKSGTSATLGITGLQTGDEADYYCGTWDSS | |
| | LRGWVFGGGTKLTVL | |
| 12 | QSVVTQPPSMSAAPGQQVTISCSGGNSNIERNYVSWYLQLPGTAPKL | GPRC5D-205 |
| | VIFDNDRRPSGIPDRFSGSKSGTSATLGITGLQTGDEADYYCGTWDS | VL/VH |
| | SLRGWVFGGGTKLTVLGSRGGGGSGGGGSGGGGSLEMAEVQLVESGG | |
| | GLIQPGGSLRLSCAASGFTFSNYAMNWVRQAPGKGLEWVSTINGRGS | |
| | STIYADSVKGRFTISRDNSKNTLYLQMNSLRAEDTATYYCARYISRG | |
| | LGDSWGQGTLVTV | |
| 13 | QVQLVQSGAEVKKPGASVKVSCKASGYTFTSYYMHWVRQAPGQGLEW | GPRC5D-206 |
| | MGIINPSGGSTRYAQKFQGRVTMTRDTSTSTVYMELSSLRSEDTAVY | VH/VL |
| | YCARGSSRWGGWTGDYWGQGTLVTVSSGSRGGGGSGGGGSGGGGSLE | |
| | MAQSALTQPASVSGSPGQSITISCTGTSSDVGGYNFVSWYQQHPGKA | |
| | PKVMIYDVSKRPSGISNRFSGSKSGNTASLTISGLQVEDEAEYYCSS | |
| | YTSTRTVIFAGGTKVTVL | |
| 14 | QSALTQPASVSGSPGQSITISCTGTSSDVGGYNFVSWYQQHPGKAPK | GPRC5D-206 |
| | VMIYDVSKRPSGISNRFSGSKSGNTASLTISGLQVEDEAEYYCSSYT | VL/VH |
| | STRTVIFAGGTKVTVLGSRGGGGSGGGGSGGGGSLEMAQVQLVQSGA | |
| | EVKKPGASVKVSCKASGYTFTSYYMHWVRQAPGQGLEWMGIINPSGG | |
| | STRYAQKFQGRVTMTRDTSTSTVYMELSSLRSEDTAVYYCARGSSRW | |
| | GGWTGDYWGQGTLVTVSS | |
| 15 | ESKYGPPCPPCP | short spacer |
| 16 | ESKYGPPCPPCPGQPREPQVYTLPPSQEEMTKNQVSLTCLVKGFYPS | medium spacer |
| | DIAVEWESNGQPENNYKTTPPVLDSDGSFFLYSRLTVDKSRWQEGNV | (hinge-CH3 119 |
| | FSCSVMHEALHNHYTQKSLSLSLGK | aa) |
| 17 | ESKYGPPCPPCPAPPVAGPSVFLFPPKPKDTLMISRTPEVTCVVVDV | long spacer |
| | SQEDPEVQFNWYVDGVEVHNAKTKPREEQFQSTYRVVSVLTVLHQDW | (IgG4/IgG2 hinge- |
| | LNGKEYKCKVSNKGLPSSIEKTISKAKGQPREPQVYTLPPSQEEMTK | IgG2/IgG4 CH2- |
| | NQVSLTCLVKGFYPSDIAVEWESNGQPENNYKTTPPVLDSDGSFFLY | IgG4 CH3 spacer; |
| | SRLTVDKSRWQEGNVFSCSVMHEALHNHYTQKSLSLSLGK | 228 aa) |
| 18 | MFWVLVVVGGVLACYSLLVTVAFIIFWV | CD28 |
| | | transmembrane |
| | | domain |
| 19 | KRGRKKLLYIFKQPFMRPVQTTQEEDGCSCRFPEEEEGGCEL | 4-1BB-derived |
| | | intracellular co- |
| | | signaling sequence |
| | | (aa) |
| 20 | RVKFSRSADAPAYQQGQNQLYNELNLGRREEYDVLDKRRGRDPEMGG | CD3-zeta derived |
| | KPRRKNPQEGLYNELQKDKMAEAYSEIGMKGERRRGKGHDGLYQGLS | intracellular |
| | TATKDTYDALHMQALPPR | signaling domain |
| | | (aa) |
| 21 | EVQLVESGGAFVQPGGSLRLSCAASGFTFSSYAMTWVRQAPGKGLEW | GPRC5D-200 VH |
| | VSTISGRGRSTFYADSVKGRFTISRDNSKNTLYLQMNSLRAEDTAVY | |
| | YCARYYHAGAFDLWGQGTLVTVSS | |
| 22 | QSVVTQPASVSGSPGQSITISCTGTSSDVGGYNYVSWYQQHPGKAPK | GPRC5D-200 VL |
| | LMIYDVSKRPSGVSNRFSGSKSGNTASLTISGLQAEDEADYYCSSYT | |
| | SSSTLVFGGGTKLTVLG | |
| 23 | QMQLVQSGAEVKKPGASVKVSCKASGYTFNRYAITWVRQAPGQGLEW | GPRC5D-201 VH |
| | MGWISAYNGNSHYAQKLQGRVTMTTDTSTGTAYMELRRLRSDDTAVY | |
| | YCARMAYDSWGQGTLVTVSS | |
| 24 | QSVLTQPASVSGSPGQSLTISCTGTSNDVGAYKYVSWYQQYPGKAPK | GPRC5D-201 VL |
| | LILYDVFKRPSGVSNRFSGSKSDNTASLTISGLQAEDEADYYCFSLT | |
| | SSNTYVFGTGTKVTVLG | |
| 25 | EVQLVESGGGLVKPGGSLRLSCAASGFTFSDYYMSWIRQAPGKGLEW | GPRC5D-202 VH |
| | VSYISSSGSTIYYADSVKGRFTISRDNAKNSLYLQMNSLRAEDTAVY | |
| | YCARGYGKAYDQWGQGTLVTVSS | |
| 26 | QSVLTQPPSASGTPGQRVTISCSGSRSNVGGNYVFWYQQVPGATPKL | GPRC5D-202 VL |
| | LIYRSNQRPSGVPDRFAGSKSGSSASLAISGLRSEDEADYYCATWDD | |
| | SLSGFVFGTGTKVTVLG | |
| 27 | QVQLVESGGGLVHPGGSLRLSCAASGFTFRSHSMNWVRQAPGKGLEW | GPRC5D-203 VH |
| | VSSISSDSTYTYYADSVKGRFTISRDNAKNSLYLQMNSLRAEDTAVY | |
| | YCARSGGQWKYYDYWGQGTLVTVSS | |
| 28 | SSELTQDPAVSVALGQTVRITCQGDSLRSYYASWYQQKPGQAPVLVI | GPRC5D-203 VL |
| | YGKNNRPSGIPDRFSGSSSGNTASLTITGAQAEDEADYYCNSRDSSG | |
| | NPPVVFGGGTKLTVLG | |
| 29 | EVQLVESGGGLVQPGGSLRLSCAASGFTFSNYAMSWVRQAPGKGLEW | GPRC5D-204 VH |
| | VSAISGSGNTYYADSVKGRFTISRDNSKNTLYLQMNSLRAEDTAVYY | |
| | CARGSVRYTDIWGQGTLVTVSS | |
| 30 | NFMLTQPHSVSESPGKTVSISCTRTSGAIAGAYVQWFQQRPGSAPTT | GPRC5D-204 VL |
| | VIYDDNKRPSGVPDRFSGSIDKSSNSASLTISGLKTEDEADYYCQSY | |
| | DYDSSNVLFGGGTKLTVLG | |
| 31 | EVQLVESGGGLIQPGGSLRLSCAASGFTFSNYAMNWVRQAPGKGLEW | GPRC5D-205 VH |
| | VSTINGRGSSTIYADSVKGRFTISRDNSKNTLYLQMNSLRAEDTATY | |
| | YCARYISRGLGDSWGQGTLVTV | |
| 32 | QSVVTQPPSMSAAPGQQVTISCSGGNSNIERNYVSWYLQLPGTAPKL | GPRC5D-205 VL |
| | VIFDNDRRPSGIPDRFSGSKSGTSATLGITGLQTGDEADYYCGTWDS | |
| | SLRGWVFGGGTKLTVLG | |
| 33 | QVQLVQSGAEVKKPGASVKVSCKASGYTFTSYYMHWVRQAPGQGLEW | GPRC5D-206 VH |
| | MGIINPSGGSTRYAQKFQGRVTMTRDTSTSTVYMELSSLRSEDTAVY | |
| | YCARGSSRWGGWTGDYWGQGTLVTVSS | |
| 34 | QSALTQPASVSGSPGQSITISCTGTSSDVGGYNFVSWYQQHPGKAPK | GPRC5D-206 VL |
| | VMIYDVSKRPSGISNRFSGSKSGNTASLTISGLQVEDEAEYYCSSYT | |
| | STRTVIFAGGTKVTVLG | |
| 35 | EGRGSLLTCGDVEENPGP | T2A peptide (aa) |
| 36 | GSGEGRGSLLTCGDVEENPGP | T2A peptide (aa) |
| 37 | LEGGGEGRGSLLTCGDVEENPGPR | T2A peptide (aa) |
| 38 | ATNFSLLKQAGDVEENPGP | P2A peptide (aa) |
| 39 | GSGATNFSLLKQAGDVEENPGP | P2A peptide (aa) |
| 40 | QCTNYALLKLAGDVESNPGP | E2A peptide (aa) |
| 41 | GSGQCTNYALLKLAGDVESNPGP | E2A peptide (aa) |
| 42 | VKQTLNFDLLKLAGDVESNPGP | F2A peptide (aa) |
| 43 | GSGVKQTLNFDLLKLAGDVESNPGP | F2A peptide (aa) |
| 44 | ctcgagggcggcggagagggcagaggaagtcttctaacatgcggtga | T2A peptide (nt) |
| | cgtggaggagaatcccggccctagg | |
| 45 | cttgaaggtggtggcgaaggcagaggcagcctgcttacatgcggaga | T2A peptide (nt) |
| | tgtggaagagaaccccggacctaga | |
| 46 | RSKRSRLLHSDYMNMTPRRPGPTRKHYQPYAPPRDFAAYRS | CD28 co-stimulatory |
| | | domain |
| 47 | GSRGGGGSGGGGSGGGGSLEMA | linker |
| 48 | gagtctaaatacggaccgccttgtcctccttgtcccgctcctcctgt | long spacer (nt) |
| | tgccggaccttccgtgttcctgtttcctccaaagcctaaggacaccc | |
| | tgatgatcagcaggacccctgaagtgacctgcgtggtggtggatgtg | |
| | tcccaagaggatcccgaggtgcagttcaactggtatgtggacggcgt | |
| | ggaagtgcacaacgccaagaccaagcctagagaggaacagttccaga | |
| | gcacctacagagtggtgtccgtgctgacagtgctgcaccaggattgg | |
| | ctgaacggcaaagagtacaagtgcaaggtgtccaacaagggcctgcc | |
| | tagcagcatcgag | |
| | aaaaccatctccaaggccaagggccagccaagagagccccaggttta | |
| | cacactgcctccaagccaagaggaaatgaccaagaatcaggtgtccc | |
| | tgacatgcctggtcaagggcttctacccctccgatatcgccgtggaa | |
| | tgggagagcaatggccagcctgagaacaactacaagaccacacctcc | |
| | tgtgctggacagcgacggcagtttcttcctgtatagtagactcaccg | |
| | tggataaatcaagatggcaagagggcaacgtgttcagctgcagcgtg | |
| | atgcacgaggccctgcacaaccactacacccagaaaagcctgagcct | |
| | gtctctgggcaag | |
| 49 | MYKDCIESTGDYFLLCDAEGPWGIILESLAILGIVVTILLLLAFLFL | GPRC5D protein |
| | MRKIQDCSQWNVLPTQLLFLLSVLGLFGLAFAFIIELNQQTAPVRYF | (Uniprot |
| | LFGVLFALCFSCLLAHASNLVKLVRGCVSFSWTTILCIAIGCSLLQI | Q9NZD1) |
| | IIATEYVTLIMTRGMMFVNMTPCQLNVDFVVLLVYVLFLMALTFFVS | |
| | KATFCGPCENWKQHGRLIFITVLFSIIIWVVWISMLLRGNPQFQRQP | |
| | QWDDPVVCIALVTNAWVFLLLYIVPELCILYRSCRQECPLQGNACPV | |
| | TAYQHSFQVENQELSRARDSDGAEEDVALTSYGTPIQPQTVDPTQEC | |
| | FIPQAKLSPQQDAGGV | |
| 50 | GGGGS | 4GS linker |
| 51 | GGGS | 3GSlinker |
| 52 | GGGGSGGGGSGGGGS | linker |
| 53 | GSTSGSGKPGSGEGSTKG | linker |
| 54 | SRGGGGSGGGGSGGGGSLEMA | linker |
| 55 | atgttttgggtgctggtcgtggtcggaggggtgctggcctgttacag | CD28 |
| | cctgctggtgacagtcgctttcatcatcttctgggtg | transmembrane |
| | | domain (nt)- |
| | | not optimized |
| 56 | atgttctgggtgctcgtggtcgttggcggagtgctggcctgttacag | CD28 |
| | cctgctggttaccgtggccttcatcatcttttgggtc | transmembrane |
| | | domain (nt)- |
| | | optimized |
| 57 | agagtcaagttttccaggtccgccgacgctccagcctaccagcaggg | CD3-zeta derived |
| | gcagaaccagctgtacaacgagctgaacctgggcagaagggaagagt | intracellular |
| | acgacgtcctggataagcggagaggccgggaccctgagatgggcggc | signaling domain |
| | aagcctcggcggaagaacccccaggaaggcctgtataacgaactgca | (nt) |
| | gaaagacaagatggccgaggcctacagcgagatcggcatgaagggcg | |
| | agcggaggcggggcaagggccacgacggcctgtatcagggcctgtcc | |
| | accgccaccaaggatacctacgacgccctgcacatgcaggccctgcc | |
| | cccaagg | |
| 58 | agagtgaagttcagcagatccgccgacgctccagcctatcagcaggg | CD3-zeta derived |
| | ccaaaaccagctgtacaacgagctgaacctggggagaagagaagagt | intracellular |
| | acgacgtgctggataagcggagaggcagagatcctgaaatgggcggc | signaling domain |
| | aagcccagacggaagaatcctcaagagggcctgtataatgagctgca | (nt)- optimized |
| | gaaagacaagatggccgaggcctacagcgagatcggaatgaagggcg | |
| | agcgcagaagaggcaagggacacgatggactgtaccagggcctgagc | |
| | accgccaccaaggatacctatgacgcactgcacatgcaggccctgcc | |
| | acctaga | |
| 59 | aagcgggggagaaagaaactgctgtatattttcaaacagccctttat | 4-1BB-derived |
| | gagacctgtgcagactacccaggaggaagacggatgcagctgtaggt | intracellular co- |
| | ttcccgaggaagaggaaggaggctgtgagctg | signaling sequence |
| | | (nt) |
| 60 | aagcggggcagaaagaagctgctctacatcttcaagcagcccttcat | 4-1BB-derived |
| | gcggcccgtgcagaccacacaagaggaagatggctgctcctgcagat | intracellular co- |
| | tccccgaggaagaagaaggcggctgcgagctg | signaling sequence |
| | | (nt)- optimized |
| 61 | ggatctgcgatcgctccggtgcccgtcagtgggcagagcgcacatcg | EF1alpha promoter |
| | cccacagtccccgagaagttggggggaggggtcggcaattgaaccgg | with HTLV1 |
| | tgcctagagaaggtggcgcggggtaaactgggaaagtgatgtcgtgt | ehancer |
| | actggctccgcctttttcccgagggtgggggagaaccgtatataagt | |
| | gcagtagtcgccgtgaacgttctttttcgcaacgggtttgccgccag | |
| | aacacagctgaagcttcgaggggctcgcatctctccttcacgcgccc | |
| | gccgccctacctgaggccgccatccacgccggttgagtcgcgttctg | |
| | ccgcctcccgcctgtggtgcctcctgaactgcgtccgccgtctaggt | |
| | aagtttaaagctcaggtcgagaccgggcctttgtccggcgctccctt | |
| | ggagcctacctagactcagccggctctccacgctttgcctgaccctg | |
| | cttgctcaactctacgtctttgtttcgttttctgttctgcgccgtta | |
| | cagatccaagctgtgaccggcgcctac | |
| 62 | aatcaacctctggattacaaaatttgtgaaagattgactggtattct | Woodchuck |
| | taactatgttgctccttttacgctatgtggatacgctgctttaatgc | Hepatitis Virus |
| | ctttgtatcatgctattgcttcccgtatggctttcattttctcctcc | (WHP) |
| | ttgtataaatcctggttgctgtctctttatgaggagttgtggcccgt | Posttranscriptional |
| | tgtcaggcaacgtggcgtggtgtgcactgtgtttgctgacgcaaccc | Regulatory |
| | ccactggttggggcattgccaccacctgtcagctcctttccgggact | Element (WPRE) |
| | ttcgctttccccctccctattgccacggcggaactcatcgccgcctg | |
| | ccttgcccgctgctggacaggggctcggctgttgggcactgacaatt | |
| | ccgtggtgttgtcggggaaatcatcgtcctttccttggctgctcgcc | |
| | tgtgttgccacctggattctgcgcgggacgtccttctgctacgtccc | |
| | ttcggccctcaatccagcggaccttccttcccgcggcctgctgccgg | |
| | ctctgcggcctcttccgcgtcttcgccttcgccctcagacgagtcgg | |
| | atctccctttgggccgcctccccgc | |
| 63 | QSVVTQPASVSGSPGQSITISCTGTSSDVGGYNYVSWYQQHPGKAPK | GPRC5D-200 VL |
| | LMIYDVSKRPSGVSNRFSGSKSGNTASLTISGLQAEDEADYYCSSYT | |
| | SSSTLVFGGGTKLTVL | |
| 64 | QSVLTQPASVSGSPGQSLTISCTGTSNDVGAYKYVSWYQQYPGKAPK | GPRC5D-201 VL |
| | LILYDVFKRPSGVSNRFSGSKSDNTASLTISGLQAEDEADYYCFSLT | |
| | SSNTYVFGTGTKVTVL | |
| 65 | QSVLTQPPSASGTPGQRVTISCSGSRSNVGGNYVFWYQQVPGATPKL | GPRC5D-202 VL |
| | LIYRSNQRPSGVPDRFAGSKSGSSASLAISGLRSEDEADYYCATWDD | |
| | SLSGFVFGTGTKVTVL | |
| 66 | SSELTQDPAVSVALGQTVRITCQGDSLRSYYASWYQQKPGQAPVLVI | GPRC5D-203 VL |
| | YGKNNRPSGIPDRFSGSSSGNTASLTITGAQAEDEADYYCNSRDSSG | |
| | NPPVVFGGGTKLTVL | |
| 67 | NFMLTQPHSVSESPGKTVSISCTRTSGAIAGAYVQWFQQRPGSAPTT | GPRC5D-204 VL |
| | VIYDDNKRPSGVPDRFSGSIDKSSNSASLTISGLKTEDEADYYCQSY | |
| | DYDSSNVLFGGGTKLTVL | |
| 68 | QSVVTQPPSMSAAPGQQVTISCSGGNSNIERNYVSWYLQLPGTAPKL | GPRC5D-205 VL |
| | VIFDNDRRPSGIPDRFSGSKSGTSATLGITGLQTGDEADYYCGTWDS | |
| | SLRGWVFGGGTKLTVL | |
| 69 | QSALTQPASVSGSPGQSITISCTGTSSDVGGYNFVSWYQQHPGKAPK | GPRC5D-206 VL |
| | VMIYDVSKRPSGISNRFSGSKSGNTASLTISGLQVEDEAEYYCSSYT | |
| | STRTVIFAGGTKVTVL | |
| 70 | ATGCCGCTGCTGCTACTGCTGCCCCTGCTGTGGGCAGGGGCTCTAGC | anti-GPRC5D |
| | TTCTTCTGAGCTGACCCAAGATCCTGCCGTGTCTGTGGCTCTGGGCC | CAR nt |
| | AGACAGTGCGGATTACCTGTCAGGGCGATAGCCTGAGAAGCTACTAC | |
| | GCCAGCTGGTATCAGCAGAAGCCTGGACAGGCTCCCGTGCTGGTCAT | |
| | CTACGGCAAGAACAACAGACCCAGCGGCATCCCCGATAGATTCAGCG | |
| | GAAGCAGCTCTGGCAATACCGCCAGCCTGACAATTACTGGCGCCCAG | |
| | GCCGAAGATGAGGCCGACTACTACTGCAACAGCAGAGACAGCTCCGG | |
| | CAATCCTCCTGTGGTTTTTGGCGGAGGCACCAAGCTGACAGTGCTGG | |
| | GAAGTAGAGGTGGCGGAGGATCTGGCGGCGGAGGAAGCGGAGGCGGC | |
| | GGATCTCTTGAAATGGCTCAGGTGCAGCTGGTGGAATCAGGCGGTGG | |
| | ACTTGTTCACCCTGGCGGAAGCCTGAGACTGTCTTGTGCCGCCAGCG | |
| | GCTTCACCTTCAGATCCCACAGCATGAACTGGGTCCGACAGGCCCCT | |
| | GGCAAAGGCCTTGAATGGGTGTCCAGCATCAGCAGCGACAGCACCTA | |
| | CACCTACTATGCCGACAGCGTGAAGGGCAGATTCACCATCTCCAGAG | |
| | ACAACGCCAAGAACAGCCTGTACCTGCAGATGAACTCCCTGAGAGCC | |
| | GAGGACACCGCCGTGTACTACTGTGCTAGAAGTGGCGGCCAGTGGAA | |
| | GTACTACGACTATTGGGGCCAGGGCACCCTGGTCACAGTTAGCTCTG | |
| | AGTCTAAATACGGACCGCCTTGTCCTCCTTGTCCCGCTCCTCCTGTT | |
| | GCCGGACCTTCCGTGTTCCTGTTTCCTCCAAAGCCTAAGGACACCCT | |
| | GATGATCAGCAGGACCCCTGAAGTGACCTGCGTGGTGGTGGATGTGT | |
| | CCCAAGAGGATCCCGAGGTGCAGTTCAACTGGTATGTGGACGGCGTG | |
| | GAAGTGCACAACGCCAAGACCAAGCCTAGAGAGGAACAGTTCCAGAG | |
| | CACCTACAGAGTGGTGTCCGTGCTGACAGTGCTGCACCAGGATTGGC | |
| | TGAACGGCAAAGAGTACAAGTGCAAGGTGTCCAACAAGGGCCTGCCT | |
| | AGCAGCATCGAGAAAACCATCTCCAAGGCCAAGGGCCAGCCAAGAGA | |
| | GCCCCAGGTTTACACACTGCCTCCAAGCCAAGAGGAAATGACCAAGA | |
| | ATCAGGTGTCCCTGACATGCCTGGTCAAGGGCTTCTACCCCTCCGAT | |
| | ATCGCCGTGGAATGGGAGAGCAATGGCCAGCCTGAGAACAACTACAA | |
| | GACCACACCTCCTGTGCTGGACAGCGACGGCAGTTTCTTCCTGTATA | |
| | GTAGACTCACCGTGGATAAATCAAGATGGCAAGAGGGCAACGTGTTC | |
| | AGCTGCAGCGTGATGCACGAGGCCCTGCACAACCACTACACCCAGAA | |
| | AAGCCTGAGCCTGTCTCTGGGCAAGATGTTCTGGGTGCTCGTGGTCG | |
| | TTGGCGGAGTGCTGGCCTGTTACAGCCTGCTGGTTACCGTGGCCTTC | |
| | ATCATCTTTTGGGTCAAGCGGGGCAGAAAGAAGCTGCTCTACATCTT | |
| | CAAGCAGCCCTTCATGCGGCCCGTGCAGACCACACAAGAGGAAGATG | |
| | GCTGCTCCTGCAGATTCCCCGAGGAAGAAGAAGGCGGCTGCGAGCTG | |
| | AGAGTGAAGTTCAGCAGATCCGCCGACGCTCCAGCCTATCAGCAGGG | |
| | CCAAAACCAGCTGTACAACGAGCTGAACCTGGGGAGAAGAGAAGAGT | |
| | ACGACGTGCTGGATAAGCGGAGAGGCAGAGATCCTGAAATGGGCGGC | |
| | AAGCCCAGACGGAAGAATCCTCAAGAGGGCCTGTATAATGAGCTGCA | |
| | GAAAGACAAGATGGCCGAGGCCTACAGCGAGATCGGAATGAAGGGCG | |
| | AGCGCAGAAGAGGCAAGGGACACGATGGACTGTACCAGGGCCTGAGC | |
| | ACCGCCACCAAGGATACCTATGACGCACTGCACATGCAGGCCCTGCC | |
| | ACCTAGA | |
| 71 | MPLLLLLPLLWAGALA | CD33 signal |
| | | sequence |
| 72 | SSELTQDPAVSVALGQTVRITCQGDSLRSYYASWYQQKPGQAPVLVI | anti-GPRC5D |
| | YGKNNRPSGIPDRFSGSSSGNTASLTITGAQAEDEADYYCNSRDSSG | CAR |
| | NPPVVFGGGTKLTVLGSRGGGGSGGGGSGGGGSLEMAQVQLVESGGG | |
| | LVHPGGSLRLSCAASGFTFRSHSMNWVRQAPGKGLEWVSSISSDSTY | |
| | TYYADSVKGRFTISRDNAKNSLYLQMNSLRAEDTAVYYCARSGGQWK | |
| | YYDYWGQGTLVTVSSESKYGPPCPPCPAPPVAGPSVFLFPPKPKDTL | |
| | MISRTPEVTCVVVDVSQEDPEVQFNWYVDGVEVHNAKTKPREEQFQS | |
| | TYRVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQK | |
| | SLSLSLGKMFWVLVVVGGVLACYSLLVTVAFIIFWVKRGRKKLLYIF | |
| | KQPFMRPVQTTQEEDGCSCRFPEEEEGGCELRVKFSRSADAPAYQQG | |
| | QNQLYNELNLGRREEYDVLDKRRGRDPEMGGKPRRKNPQEGLYNELQ | |
| | KDKMAEAYSEIGMKGERRRGKGHDGLYQGLSTATKDTYDALHMQALP | |
| | PR | |
| 73 | gaatctaagtacggaccgccctgccctccctgccctgctcctcctgt | IgG4/IgG2 hinge- |
| | ggctggaccaagcgtgttcctgtttccacctaagcctaaagataccc | IgG2/IgG4 CH2- |
| | tgatgatttcccgcacacctgaagtgacttgcgtggtcgtggacgtg | IgG4 CH3 spacer |
| | agccaggaggatccagaagtgcagttcaactggtacgtggacggcgt | (nt) |
| | ggaagtccacaatgctaagactaaaccccgagaggaacagtttcagt | |
| | caacttaccgggtcgtgagcgtgctgaccgtcctgcatcaggattgg | |
| | ctgaacgggaaggagtataagtgcaaagtgtctaataagggactgcc | |
| | tagctccatcgagaaaacaattagtaaggcaaaagggcagcctcgag | |
| | aaccacaggtgtataccctgccccctagccaggaggaaatgaccaag | |
| | aaccaggtgtccctgacatgtctggtcaaaggcttctatccaagtac | |
| | atcgccgtggagtgggaatcaaatgggcagcccgagaacaattacaa | |
| | gaccacaccacccgtgctggactctgatggaagtttctttctgtatt | |
| | ccaggctgaccgtggataaatctcgctggcaggagggcaacgtgttc | |
| | tcttgcagtgtcatgcacgaagccctgcacaatcattatacacagaa | |
| | gtcactgagcctgtccctgggcaaa | |
| 74 | gagtctaaatacggaccgccttgtcctccttgtcccgctcctcctgt | optimized SSE |
| | tgccggaccttccgtgttcctgtttcctccaaagcctaaggacaccc | IgG4/IgG2 hinge- |
| | tgatgatcagcaggacccctgaagtgacctgcgtggtggtggatgtg | IgG2/IgG4 CH2- |
| | tcccaagaggatcccgaggtgcagttcaactggtatgtggacggcgt | IgG4 CH3 spacer |
| | ggaagtgcacaacgccaagaccaagcctagagaggaacagttccaga | (nt) |
| | gcacctacagagtggtgtccgtgctgacagtgctgcaccaggattgg | |
| | ctgaacggcaaagagtacaagtgcaaggtgtccaacaagggcctgcc | |
| | tagcagcatcgagaaaaccatctccaaggccaagggccagccaagag | |
| | agccccaggtttacacactgcctccaagccaagaggaaatgaccaag | |
| | aatcaggtgtccctgacatgcctggtcaagggcttctacccctccga | |
| | tatcgccgtggaatgggagagcaatggccagcctgagaacaactaca | |
| | agaccacacctcctgtgctggacagcgacggcagtttcttcctgtat | |
| | agtagactcaccgtggataaatcaagatggcaagagggcaacgtgtt | |
| | cagctgcagcgtgatgcacgaggccctgcacaaccactacacccaga | |
| | aaagcctgagcctgtctctgggcaag | |
| 75 | GFTFSSY | CDRH1 |
| 76 | SGRGRS | CDRH2 |
| 77 | YYHAGAFDL | CDRH3 |
| 78 | GFTFSSYAMT | CDRH1 |
| 79 | TISGRGRSTF | CDRH2 |
| 80 | SYAMT | CDRH1 |
| 81 | TISGRGRSTFYADSVKG | CDRH2 |
| 82 | GFTFSSYA | CDRH1 |
| 83 | ISGRGRST | CDRH2 |
| 84 | ARYYHAGAFDL | CDRH3 |
| 85 | TGTSSDVGGYNYVS | CDRL1 |
| 86 | DVSKRPS | CDRL2 |
| 87 | SSYTSSSTLV | CDRL3 |
| 88 | SSDVGGYNY | CDRL1 |
| 89 | DVS | CDRL2 |
| 90 | GYTFNRY | CDRH1 |
| 91 | SAYNGN | CDRH2 |
| 92 | MAYDS | CDRH3 |
| 93 | GYTFNRYAIT | CDRH1 |
| 94 | WISAYNGNSH | CDRH2 |
| 95 | RYAIT | CDRH1 |
| 96 | WISAYNGNSHYAQKLQG | CDRH2 |
| 97 | GYTFNRYA | CDRH1 |
| 98 | ISAYNGNS | CDRH2 |
| 99 | ARMAYDS | CDRH3 |
| 100 | TGTSNDVGAYKYVS | CDRL1 |
| 101 | DVFKRPS | CDRL2 |
| 102 | FSLTSSNTYV | CDRL3 |
| 103 | SNDVGAYKY | CDRL1 |
| 104 | DVF | CDRL2 |
| 105 | GFTFSDY | CDRH1 |
| 106 | SSSGST | CDRH2 |
| 107 | GYGKAYDQ | CDRH3 |
| 108 | GFTFSDYYMS | CDRH1 |
| 109 | YISSSGSTIY | CDRH2 |
| 110 | DYYMS | CDRH1 |
| 111 | YISSSGSTIYYADSVKG | CDRH2 |
| 112 | GFTFSDYY | CDRH1 |
| 113 | ISSSGSTI | CDRH2 |
| 114 | ARGYGKAYDQ | CDRH3 |
| 115 | SGSRSNVGGNYVF | CDRL1 |
| 116 | RSNQRPS | CDRL2 |
| 117 | ATWDDSLSGFV | CDRL3 |
| 118 | RSNVGGNY | CDRL1 |
| 119 | RSN | CDRL2 |
| 120 | GFTFRSH | CDRH1 |
| 121 | SSDSTY | CDRH2 |
| 122 | SGGQWKYYDY | CDRH3 |
| 123 | GFTFRSHSMN | CDRH1 |
| 124 | SISSDSTYTY | CDRH2 |
| 125 | SHSMN | CDRH1 |
| 126 | SISSDSTYTYYADSVKG | CDRH2 |
| 127 | GFTFRSHS | CDRH1 |
| 128 | ISSDSTYT | CDRH2 |
| 129 | ARSGGQWKYYDY | CDRH3 |
| 130 | QGDSLRSYYAS | CDRL1 |
| 131 | GKNNRPS | CDRL2 |
| 132 | NSRDSSGNPPVV | CDRL3 |
| 133 | SLRSYY | CDRL1 |
| 134 | GKN | CDRL2 |
| 135 | GFTFSNY | CDRH1 |
| 136 | SGSGN | CDRH2 |
| 137 | GSVRYTDI | CDRH3 |
| 138 | GFTFSNYAMS | CDRH1 |
| 139 | AISGSGNTY | CDRH2 |
| 140 | NYAMS | CDRH1 |
| 141 | AISGSGNTYYADSVKG | CDRH2 |
| 142 | GFTFSNYA | CDRH1 |
| 143 | ISGSGNT | CDRH2 |
| 144 | ARGSVRYTDI | CDRH3 |
| 145 | TRTSGAIAGAYVQ | CDRL1 |
| 146 | DDNKRPS | CDRL2 |
| 147 | QSYDYDSSNVL | CDRL3 |
| 148 | SGAIAGAY | CDRL1 |
| 149 | DDN | CDRL2 |
| 150 | NGRGSS | CDRH2 |
| 151 | YISRGLGDS | CDRH3 |
| 152 | GFTFSNYAMN | CDRH1 |
| 153 | TINGRGSSTI | CDRH2 |
| 154 | TINGRGSSTIYADSVKG | CDRH2 |
| 155 | INGRGSST | CDRH2 |
| 156 | ARYISRGLGDS | CDRH3 |
| 157 | SGGNSNIERNYVS | CDRL1 |
| 158 | DNDRRPS | CDRL2 |
| 159 | GTWDSSLRGWV | CDRL3 |
| 160 | NSNIERNY | CDRL1 |
| 161 | DND | CDRL2 |
| 162 | GYTFTSY | CDRH1 |
| 163 | NPSGGS | CDRH2 |
| 164 | GSSRWGGWTGDY | CDRH3 |
| 165 | GYTFTSYYMH | CDRH1 |
| 166 | IINPSGGSTR | CDRH2 |
| 167 | SYYMH | CDRH1 |
| 168 | IINPSGGSTRYAQKFQG | CDRH2 |
| 169 | GYTFTSYY | CDRH1 |
| 170 | INPSGGST | CDRH2 |
| 171 | ARGSSRWGGWTGD | CDRH3 |
| 172 | TGTSSDVGGYNFVS | CDRL1 |
| 173 | SSYTSTRTVI | CDRL3 |
| 174 | SSDVGGYNF | CDRL1 |
| 175 | SSYTSTRTVIFAGGTKVTVL | CDRL3 |
| 176 | aatctaagtacggac | splice donor site |
| 177 | tcaactggtacgtgg | splice donor site |
| 178 | acaattagtaaggca | splice donor site |
| 179 | accacaggtgtatac | splice donor site |
| 180 | aagtttctttctgtattccaggctgaccgtggataaatctc | splice acceptor site |
| 181 | gggcaacgtgttctcttgcagtgtcatgcacgaagccctgc | splice acceptor site |
| 182 | agtctaaatacggac | modified splice |
| | | donor site |
| 183 | tcaactggtatgtgg | modified splice |
| | | donor site |
| 184 | accatctccaaggcc | modified splice |
| | | donor site |
| 185 | gccccaggtttacac | modified splice |
| | | donor site |
| 186 | cagtttcttcctgtatagtagactcaccgtggataaatcaa | modified splice |
| | | acceptor site |
| 187 | gggcaacgtgttcagctgcagcgtgatgcacgaggccctgc | modified splice |
| | | acceptor site |
| 188 | cgccttgtcctccttgtcccgctcctcctgttgccggacct | modified splice |
| | | acceptor site |
| 189 | EVQLVESGGGLVKPGGSLRLSCAASGFTFSDYYMSWIRQAPGKGLEW | BCMA-23 VH |
| | VSYISSSGSTIYYADSVKGRFTISRDNAKNSLYLQMNSLRAEDTAVY | |
| | YCAKVDGDYTEDYWGQGTLVTVSS | |
| 190 | QSALTQPASVSGSPGQSITISCTGSSSDVGKYNLVSWYQQPPGKAPK | BCMA-23 VL |
| | LIIYDVNKRPSGVSNRFSGSKSGNTATLTISGLQGDDEADYYCSSYG | |
| | GSRSYVFGTGTKVTVL | |
| 191 | EVQLVQSGGGLVQPGRSLRLSCTASGFTFGDYAMSWFRQAPGKGLEW | BCMA-25 VH |
| | VGFIRSKAYGGTTEYAASVKGRFTISRDDSKSIAYLQMNSLKTEDTA | |
| | VYYCAAWSAPTDYWGQGTLVTVSS | |
| 192 | DIQMTQSPAFLSASVGDRVTVTCRASQGISNYLAWYQQKPGNAPRLL | BCMA-25 VL |
| | IYSASTLQSGVPSRFRGTGYGTEFSLTIDSLQPEDFATYYCQQSYTS | |
| | RQTFGPGTRLDIK | |
| 193 | EVQLVESGGGLVKPGGSLRLSCAASGFTFSDYYMSWIRQAPGKGLEW | BCMA-26 VH |
| | VSYISSSGSTIYYADSVKGRFTISRDNAKNSLYLQMNSLRAEDTAVY | |
| | YCAKVDGPPSFDIWGQGTMVTVSS | |
| 194 | SYVLTQPPSVSVAPGQTARITCGANNIGSKSVHWYQQKPGQAPMLVV | BCMA-26 VL |
| | YDDDDRPSGIPERFSGSNSGNTATLTISGVEAGDEADYFCHLWDRSR | |
| | DHYVFGTGTKLTVL | |
| 195 | EVQLVQSGAEVKKPGESLKISCKGSGYSFTSYWIGWVRQMPGKGLEW | BCMA-52 VH |
| | MGIIYPGDSDTRYSPSFQGHVTISADKSISTAYLQWSSLKASDTAMY | |
| | YCARYSGSFDNWGQGTLVTVSS | |
| 196 | SYELTQPPSASGTPGQRVTMSCSGTSSNIGSHSVNWYQQLPGTAPKL | BCMA-52 VL |
| | LIYTNNQRPSGVPDRFSGSKSGTSASLAISGLQSEDEADYYCAAWDG | |
| | SLNGLVFGGGTKLTVLG | |
| 197 | EVQLVQSGAEMKKPGASLKLSCKASGYTFIDYYVYWMRQAPGQGLES | BCMA-55 VH |
| | MGWINPNSGGTNYAQKFQGRVTMTRDTSISTAYMELSRLRSDDTAMY | |
| | YCARSQRDGYMDYWGQGTLVTVSS | |
| 198 | QSALTQPASVSASPGQSIAISCTGTSSDVGWYQQHPGKAPKLMIYED | BCMA-55 VL |
| | SKRPSGVSNRFSGSKSGNTASLTISGLQAEDEADYYCSSNTRSSTLV | |
| | FGGGTKLTVLG | |
| 199 | DYYMS | CDRH1 |
| 200 | YISSSGSTIYYADSVKG | CDRH2 |
| 201 | VDGDYTEDY | CDRH3 |
| 202 | DYAMS | CDRH1 |
| 203 | FIRSKAYGGTTEYAASVKG | CDRH2 |
| 204 | WSAPTDY | CDRH3 |
| 205 | VDGPPSFDI | CDRH3 |
| 206 | SYWIG | CDRH1 |
| 207 | IIYPGDSDTRYSPSFQG | CDRH2 |
| 208 | YSGSFDN | CDRH3 |
| 209 | DYYVY | CDRH1 |
| 210 | WINPNSGGTNYAQKFQG | CDRH2 |
| 211 | SQRDGYMDY | CDRH3 |
| 212 | GYSFTSYW | CDRH1 |
| 213 | IYPGDSDT | CDRH2 |
| 214 | ARYSGSFDN | CDRH3 |
| 215 | GYTFIDYY | CDRH1 |
| 216 | INPNSGGT | CDRH2 |
| 217 | ARSQRDGYMDY | CDRH3 |
| 218 | TGSSSDVGKYNLVS | CDRL1 |
| 219 | DVNKRPS | CDRL2 |
| 220 | SSYGGSRSYV | CDRL3 |
| 221 | RASQGISNYLA | CDRL1 |
| 222 | SASTLQS | CDRL2 |
| 223 | QQSYTSRQT | CDRL3 |
| 224 | GANNIGSKSVH | CDRL1 |
| 225 | DDDDRPS | CDRL2 |
| 226 | HLWDRSRDHYV | CDRL3 |
| 227 | SGTSSNIGSHSVN | CDRL1 |
| 228 | TNNQRPS | CDRL2 |
| 229 | AAWDGSLNGLV | CDRL3 |
| 230 | TGTSSDVG | CDRL1 |
| 231 | EDSKRPS | CDRL2 |
| 232 | SSNTRSSTLV | CDRL3 |
| 233 | SSNIGSHS | CDRL1 |
| 234 | TNN | CDRL2 |
| 235 | ISCTGTSSD | CDRL1 |
| 236 | EDS | CDRL2 |
| 237 | EVQLVESGGGLVKPGGSLRLSCAASGFTFSDYYMSWIRQAPGKGLEW | BCMA-23 scFv |
| | VSYISSSGSTIYYADSVKGRFTISRDNAKNSLYLQMNSLRAEDTAVY | |
| | YCAKVDGDYTEDYWGQGTLVTVSSGGGGSGGGGSGGGGSQSALTQPA | |
| | SVSGSPGQSITISCTGSSSDVGKYNLVSWYQQPPGKAPKLIIYDVNK | |
| | RPSGVSNRFSGSKSGNTATLTISGLQGDDEADYYCSSYGGSRSYVFG | |
| | TGTKVTVL | |
| 238 | EVQLVQSGGGLVQPGRSLRLSCTASGFTFGDYAMSWFRQAPGKGLEW | BCMA-25 scFv |
| | VGFIRSKAYGGTTEYAASVKGRFTISRDDSKSIAYLQMNSLKTEDTA | |
| | VYYCAAWSAPTDYWGQGTLVTVSSGGGGSGGGGSGGGGSDIQMTQSP | |
| | AFLSASVGDRVTVTCRASQGISNYLAWYQQKPGNAPRLLIYSASTLQ | |
| | SGVPSRFRGTGYGTEFSLTIDSLQPEDFATYYCQQSYTSRQTFGPGT | |
| | RLDIK | |
| 239 | EVQLVESGGGLVKPGGSLRLSCAASGFTFSDYYMSWIRQAPGKGLEW | BCMA-26 scFv |
| | VSYISSSGSTIYYADSVKGRFTISRDNAKNSLYLQMNSLRAEDTAVY | |
| | YCAKVDGPPSFDIWGQGTMVTVSSGGGGSGGGGSGGGGSSYVLTQPP | |
| | SVSVAPGQTARITCGANNIGSKSVHWYQQKPGQAPMLVVYDDDDRPS | |
| | GIPERFSGSNSGNTATLTISGVEAGDEADYFCHLWDRSRDHYVFGTG | |
| | TKLTVL | |
| 240 | SYELTQPPSASGTPGQRVTMSCSGTSSNIGSHSVNWYQQLPGTAPKL | BCMA-52 scFv |
| | LIYTNNQRPSGVPDRFSGSKSGTSASLAISGLQSEDEADYYCAAWDG | |
| | SLNGLVFGGGTKLTVLGSRGGGGSGGGGSGGGGSLEMAEVQLVQSGA | |
| | EVKKPGESLKISCKGSGYSFTSYWIGWVRQMPGKGLEWMGIIYPGDS | |
| | DTRYSPSFQGHVTISADKSISTAYLQWSSLKASDTAMYYCARYSGSF | |
| | DNWGQGTLVTVSS | |
| 241 | QSALTQPASVSASPGQSIAISCTGTSSDVGWYQQHPGKAPKLMIYED | BCMA-55 scFv |
| | SKRPSGVSNRFSGSKSGNTASLTISGLQAEDEADYYCSSNTRSSTLV | |
| | FGGGTKLTVLGSRGGGGSGGGGSGGGGSLEMAEVQLVQSGAEMKKPG | |
| | ASLKLSCKASGYTFIDYYVYWMRQAPGQGLESMGWINPNSGGTNYAQ | |
| | KFQGRVTMTRDTSISTAYMELSRLRSDDTAMYYCARSQRDGYMDYWG | |
| | QGTLVTVSS | |
| 242 | gaggtgcagctggtggagtccggaggaggcctggtgaagccaggagg | anti-BMCA CAR |
| | ctccctgaggctgtcttgcgcagccagcggcttcacctttagcgact | |
| | actatatgtcctggatcagacaggcacctggcaagggcctggagtgg | |
| | gtgagctacatcagctcctctggctccacaatctactatgccgactc | |
| | tgtgaagggccggtttaccatcagcagagataacgccaagaattccc | |
| | tgtatctgcagatgaacagcctgagggccgaggacacagccgtgtac | |
| | tattgcgccaaggtggacggcgattacaccgaggattattggggcca | |
| | gggcacactggtgaccgtgagctccggcggcggcggctctggaggag | |
| | gaggcagcggcggaggaggctcccagtctgccctgacacagccagcc | |
| | agcgtgtccggctctcccggacagtccatcacaatctcttgtaccgg | |
| | ctctagctccgacgtgggcaagtacaacctggtgtcctggtatcagc | |
| | agccccctggcaaggcccctaagctgatcatctacgatgtgaacaag | |
| | aggccatctggcgtgagcaatcgcttcagcggctccaagtctggcaa | |
| | taccgccacactgaccatcagcggcctgcagggcgacgatgaggcag | |
| | attactattgttctagctacggcggcagcagatcctacgtgttcggc | |
| | acaggcaccaaggtgaccgtgctggaatctaagtacggaccgccttg | |
| | tcctccttgtcccgctcctcctgttgccggaccttccgtgttcctgt | |
| | ttcctccaaagcctaaggacaccctgatgatcagcaggacccctgaa | |
| | gtgacctgcgtggtggtggatgtgtcccaagaggatcccgaggtgca | |
| | gttcaactggtatgtggacggcgtggaagtgcacaacgccaagacca | |
| | agcctagagaggaacagttccagagcacctacagagtggtgtccgtg | |
| | ctgacagtgctgcaccaggattggctgaacggcaaagagtacaagtg | |
| | caaggtgtccaacaagggcctgcctagcagcatcgagaaaaccatct | |
| | ccaaggccaagggccagccaagagagccccaggtttacacactgcct | |
| | ccaagccaagaggaaatgaccaagaatcaggtgtccctgacatgcct | |
| | ggtcaagggcttctacccctccgatatcgccgtggaatgggagagca | |
| | atggccagcctgagaacaactacaagaccacacctcctgtgctggac | |
| | agcgacggcagtttcttcctgtatagtagactcaccgtggataaatc | |
| | aagatggcaagagggcaacgtgttcagctgcagcgtgatgcacgagg | |
| | ccctgcacaaccactacacccagaaaagcctgagcctgtctctgggc | |
| | aagatgttctgggtgctcgtggtcgttggcggagtgctggcctgtta | |
| | cagcctgctggttaccgtggccttcatcatcttttgggtcaagcggg | |
| | gcagaaagaagctgctctacatcttcaagcagcccttcatgcggccc | |
| | gtgcagaccacacaagaggaagatggctgctcctgcagattccccga | |
| | ggaagaagaaggcggctgcgagctgagagtgaagttcagcagatccg | |
| | ccgacgctccagcctatcagcagggccaaaaccagctgtacaacgag | |
| | ctgaacctggggagaagagaagagtacgacgtgctggataagcggag | |
| | aggcagagatcctgaaatgggcggcaagcccagacggaagaatcctc | |
| | aagagggcctgtataatgagctgcagaaagacaagatggccgaggcc | |
| | tacagcgagatcggaatgaagggcgagcgcagaagaggcaagggaca | |
| | cgatggactgtaccagggcctgagcaccgccaccaaggatacctatg | |
| | acgcactgcacatgcaggccctgccacctaga | |
| 243 | gaggtgcagctggtgcagagcggaggaggcctggtgcagcctggcag | anti-BMCA CAR |
| | gtccctgcgcctgtcttgcaccgccagcggcttcacatttggcgact | |
| | atgccatgtcctggttcaagcaggcaccaggcaagggcctggagtgg | |
| | gtgggctttatccgctctaaggcctacggcggcaccacagagtatgc | |
| | cgccagcgtgaagggccggttcaccatcagccgggacgactctaaga | |
| | gcatcgcctacctgcagatgaactctctgaagaccgaggacacagcc | |
| | gtgtactattgcgcagcatggagcgccccaaccgattattggggcca | |
| | gggcaccctggtgacagtgagctccggcggcggcggctctggaggag | |
| | gaggaagcggaggaggaggatccgacatccagatgacacagtcccct | |
| | gcctttctgtccgcctctgtgggcgatagggtgaccgtgacatgtcg | |
| | cgcctcccagggcatctctaactacctggcctggtatcagcagaagc | |
| | ccggcaatgcccctcggctgctgatctacagcgcctccaccctgcag | |
| | agcggagtgccctcccggttcagaggaaccggctatggcacagagtt | |
| | ttctctgaccatcgacagcctgcagccagaggatttcgccacatact | |
| | attgtcagcagtcttacaccagccggcagacatttggccccggcaca | |
| | agactggatatcaaggagtctaaatacggaccgccttgtcctccttg | |
| | tcccgctcctcctgttgccggaccttccgtgttcctgtttcctccaa | |
| | agcctaaggacaccctgatgatcagcaggacccctgaagtgacctgc | |
| | gtggtggtggatgtgtcccaagaggatcccgaggtgcagttcaactg | |
| | gtatgtggacggcgtggaagtgcacaacgccaagaccaagcctagag | |
| | aggaacagttccagagcacctacagagtggtgtccgtgctgacagtg | |
| | ctgcaccaggattggctgaacggcaaagagtacaagtgcaaggtgtc | |
| | caacaagggcctgcctagcagcatcgagaaaaccatctccaaggcca | |
| | agggccagccaagagagccccaggtttacacactgcctccaagccaa | |
| | gaggaaatgaccaagaatcaggtgtccctgacatgcctggtcaaggg | |
| | cttctacccctccgatatcgccgtggaatgggagagcaatggccagc | |
| | ctgagaacaactacaagaccacacctcctgtgctggacagcgacggc | |
| | agtttcttcctgtatagtagactcaccgtggataaatcaagatggca | |
| | agagggcaacgtgttcagctgcagcgtgatgcacgaggccctgcaca | |
| | accactacacccagaaaagcctgagcctgtctctgggcaagatgttc | |
| | tgggtgctcgtggtcgttggcggagtgctggcctgttacagcctgct | |
| | ggttaccgtggccttcatcatcttttgggtcaagcggggcagaaaga | |
| | agctgctctacatcttcaagcagcccttcatgcggcccgtgcagacc | |
| | acacaagaggaagatggctgctcctgcagattccccgaggaagaaga | |
| | aggcggctgcgagctgagagtgaagttcagcagatccgccgacgctc | |
| | cagcctatcagcagggccaaaaccagctgtacaacgagctgaacctg | |
| | gggagaagagaagagtacgacgtgctggataagcggagaggcagaga | |
| | tcctgaaatgggcggcaagcccagacggaagaatcctcaagagggcc | |
| | tgtataatgagctgcagaaagacaagatggccgaggcctacagcgag | |
| | atcggaatgaagggcgagcgcagaagaggcaagggacacgatggact | |
| | gtaccagggcctgagcaccgccaccaaggatacctatgacgcactgc | |
| | acatgcaggccctgccacctaga | |
| 244 | gaggtgcagctggtggagtccggaggaggcctggtgaagccaggagg | anti-BMCA CAR |
| | ctctctgaggctgagctgcgcagcctccggcttcaccttttctgact | |
| | actatatgagctggatcaggcaggcaccaggcaagggcctggagtgg | |
| | gtgtcttacatcagctcctctggcagcacaatctactatgccgactc | |
| | cgtgaagggcaggttcaccatctctcgcgataacgccaagaatagcc | |
| | tgtatctgcagatgaactccctgcgggccgaggatacagccgtgtac | |
| | tattgcgccaaggtggacggccccccttcctttgatatctggggcca | |
| | gggcacaatggtgaccgtgagctccggaggaggaggatccggcggag | |
| | gaggctctggcggcggcggctctagctatgtgctgacccagccacca | |
| | tccgtgtctgtggcacctggacagacagcaaggatcacctgtggagc | |
| | aaacaatatcggcagcaagtccgtgcactggtaccagcagaagcctg | |
| | gccaggccccaatgctggtggtgtatgacgatgacgatcggcccagc | |
| | ggcatccctgagagattttctggcagcaactccggcaataccgccac | |
| | actgaccatctctggagtggaggcaggcgacgaggcagattacttct | |
| | gtcacctgtgggaccggagcagagatcactacgtgttcggcacaggc | |
| | accaagctgaccgtgctggaatctaagtacggaccgccttgtcctcc | |
| | ttgtcccgctcctcctgttgccggaccttccgtgttcctgtttcctc | |
| | caaagcctaaggacaccctgatgatcagcaggacccctgaagtgacc | |
| | tgcgtggtggtggatgtgtcccaagaggatcccgaggtgcagttcaa | |
| | ctggtatgtggacggcgtggaagtgcacaacgccaagaccaagccta | |
| | gagaggaacagttccagagcacctacagagtggtgtccgtgctgaca | |
| | gtgctgcaccaggattggctgaacggcaaagagtacaagtgcaaggt | |
| | gtccaacaagggcctgcctagcagcatcgagaaaaccatctccaagg | |
| | ccaagggccagccaagagagccccaggtttacacactgcctccaagc | |
| | caagaggaaatgaccaagaatcaggtgtccctgacatgcctggtcaa | |
| | gggcttctacccctccgatatcgccgtggaatgggagagcaatggcc | |
| | agcctgagaacaactacaagaccacacctcctgtgctggacagcgac | |
| | ggcagtttcttcctgtatagtagactcaccgtggataaatcaagatg | |
| | gcaagagggcaacgtgttcagctgcagcgtgatgcacgaggccctgc | |
| | acaaccactacacccagaaaagcctgagcctgtctctgggcaagatg | |
| | ttctgggtgctcgtggtcgttggcggagtgctggcctgttacagcct | |
| | gctggttaccgtggccttcatcatcttttgggtcaagcggggcagaa | |
| | agaagctgctctacatcttcaagcagcccttcatgcggcccgtgcag | |
| | accacacaagaggaagatggctgctcctgcagattccccgaggaaga | |
| | agaaggcggctgcgagctgagagtgaagttcagcagatccgccgacg | |
| | ctccagcctatcagcagggccaaaaccagctgtacaacgagctgaac | |
| | ctggggagaagagaagagtacgacgtgctggataagcggagaggcag | |
| | agatcctgaaatgggcggcaagcccagacggaagaatcctcaagagg | |
| | gcctgtataatgagctgcagaaagacaagatggccgaggcctacagc | |
| | gagatcggaatgaagggcgagcgcagaagaggcaagggacacgatgg | |
| | actgtaccagggcctgagcaccgccaccaaggatacctatgacgcac | |
| | tgcacatgcaggccctgccacctaga | |
| 245 | Agctatgagctgacacagcctccaagcgcctctggcacacctggaca | anti-BMCA CAR |
| | gcgagtgacaatgagctgtagcggcaccagcagcaacatcggcagcc | |
| | acagcgtgaactggtatcagcagctgcctggcacagcccctaaactg | |
| | ctgatctacaccaacaaccagcggcctagcggcgtgcccgatagatt | |
| | ttctggcagcaagagcggcacaagcgccagcctggctatttctggac | |
| | tgcagagcgaggacgaggccgactattattgtgccgcctgggacggc | |
| | tctctgaacggccttgtttttggcggaggcaccaagctgacagtgct | |
| | gggatctagaggtggcggaggatctggcggcggaggaagcggaggcg | |
| | gcggatctcttgaaatggctgaagtgcagctggtgcagtctggcgcc | |
| | gaagtgaagaagcctggcgagagcctgaagatcagctgcaaaggcag | |
| | cggctacagcttcaccagctactggatcggctgggtccgacagatgc | |
| | ctggcaaaggccttgagtggatgggcatcatctaccccggcgacagc | |
| | gacaccagatacagccctagctttcagggccacgtgaccatcagcgc | |
| | cgacaagtctatcagcaccgcctacctgcagtggtccagcctgaagg | |
| | cctctgacaccgccatgtactactgcgccagatactctggcagcttc | |
| | gacaattggggccagggcacactggtcaccgtgtccagcgagtctaa | |
| | atacggaccgccttgtcctccttgtcccgctcctcctgttgccggac | |
| | cttccgtgttcctgtttcctccaaagcctaaggacaccctgatgatc | |
| | agcaggacccctgaagtgacctgcgtggtggtggatgtgtcccaaga | |
| | ggatcccgaggtgcagttcaactggtatgtggacggcgtggaagtgc | |
| | acaacgccaagaccaagcctagagaggaacagttccagagcacctac | |
| | agagtggtgtccgtgctgacagtgctgcaccaggattggctgaacgg | |
| | caaagagtacaagtgcaaggtgtccaacaagggcctgcctagcagca | |
| | tcgagaaaaccatctccaaggccaagggccagccaagagagccccag | |
| | gtttacacactgcctccaagccaagaggaaatgaccaagaatcaggt | |
| | gtccctgacatgcctggtcaagggcttctacccctccgatatcgccg | |
| | tggaatgggagagcaatggccagcctgagaacaactacaagaccaca | |
| | cctcctgtgctggacagcgacggcagtttcttcctgtatagtagact | |
| | caccgtggataaatcaagatggcaagagggcaacgtgttcagctgca | |
| | gcgtgatgcacgaggccctgcacaaccactacacccagaaaagcctg | |
| | agcctgtctctgggcaagatgttctgggtgctcgtggtcgttggcgg | |
| | agtgctggcctgttacagcctgctggttaccgtggccttcatcatct | |
| | tttgggtcaagcggggcagaaagaagctgctctacatcttcaagcag | |
| | cccttcatgcggcccgtgcagaccacacaagaggaagatggctgctc | |
| | ctgcagattccccgaggaagaagaaggcggctgcgagctgagagtga | |
| | agttcagcagatccgccgacgctccagcctatcagcagggccaaaac | |
| | cagctgtacaacgagctgaacctggggagaagagaagagtacgacgt | |
| | gctggataagcggagaggcagagatcctgaaatgggcggcaagccca | |
| | gacggaagaatcctcaagagggcctgtataatgagctgcagaaagac | |
| | aagatggccgaggcctacagcgagatcggaatgaagggcgagcgcag | |
| | aagaggcaagggacacgatggactgtaccagggcctgagcaccgcca | |
| | ccaaggatacctatgacgcactgcacatgcaggccctgccacctaga | |
| 246 | cagtctgccctgacacagcctgccagcgttagtgctagtcccggaca | anti-BMCA CAR |
| | gtctatcgccatcagctgtaccggcaccagctctgacgttggctggt | |
| | atcagcagcaccctggcaaggcccctaagctgatgatctacgaggac | |
| | agcaagaggcccagcggcgtgtccaatagattcagcggcagcaagag | |
| | cggcaacaccgccagcctgacaattagcggactgcaggccgaggacg | |
| | aggccgattactactgcagcagcaacacccggtccagcacactggtt | |
| | tttggcggaggcaccaagctgacagtgctgggatctagaggtggcgg | |
| | aggatctggcggcggaggaagcggaggcggcggatctcttgaaatgg | |
| | ctgaagtgcagctggtgcagtctggcgccgagatgaagaaacctggc | |
| | gcctctctgaagctgagctgcaaggccagcggctacaccttcatcga | |
| | ctactacgtgtactggatgcggcaggcccctggacagggactcgaat | |
| | ctatgggctggatcaaccccaatagcggcggcaccaattacgcccag | |
| | aaattccagggcagagtgaccatgaccagagacaccagcatcagcac | |
| | cgcctacatggaactgagccggctgagatccgacgacaccgccatgt | |
| | actactgcgccagatctcagcgcgacggctacatggattattggggc | |
| | cagggaaccctggtcaccgtgtccagcgagtctaaatacggaccgcc | |
| | ttgtcctccttgtcccgctcctcctgttgccggaccttccgtgttcc | |
| | tgtttcctccaaagcctaaggacaccctgatgatcagcaggacccct | |
| | gaagtgacctgcgtggtggtggatgtgtcccaagaggatcccgaggt | |
| | gcagttcaactggtatgtggacggcgtggaagtgcacaacgccaaga | |
| | ccaagcctagagaggaacagttccagagcacctacagagtggtgtcc | |
| | gtgctgacagtgctgcaccaggattggctgaacggcaaagagtacaa | |
| | gtgcaaggtgtccaacaagggcctgcctagcagcatcgagaaaacca | |
| | tctccaaggccaagggccagccaagagagccccaggtttacacactg | |
| | cctccaagccaagaggaaatgaccaagaatcaggtgtccctgacatg | |
| | cctggtcaagggcttctacccctccgatatcgccgtggaatgggaga | |
| | gcaatggccagcctgagaacaactacaagaccacacctcctgtgctg | |
| | gacagcgacggcagtttcttcctgtatagtagactcaccgtggataa | |
| | atcaagatggcaagagggcaacgtgttcagctgcagcgtgatgcacg | |
| | aggccctgcacaaccactacacccagaaaagcctgagcctgtctctg | |
| | ggcaagatgttctgggtgctcgtggtcgttggcggagtgctggcctg | |
| | ttacagcctgctggttaccgtggccttcatcatcttttgggtcaagc | |
| | ggggcagaaagaagctgctctacatcttcaagcagcccttcatgcgg | |
| | cccgtgcagaccacacaagaggaagatggctgctcctgcagattccc | |
| | cgaggaagaagaaggcggctgcgagctgagagtgaagttcagcagat | |
| | ccgccgacgctccagcctatcagcagggccaaaaccagctgtacaac | |
| | gagctgaacctggggagaagagaagagtacgacgtgctggataagcg | |
| | gagaggcagagatcctgaaatgggcggcaagcccagacggaagaatc | |
| | ctcaagagggcctgtataatgagctgcagaaagacaagatggccgag | |
| | gcctacagcgagatcggaatgaagggcgagcgcagaagaggcaaggg | |
| | acacgatggactgtaccagggcctgagcaccgccaccaaggatacct | |
| | atgacgcactgcacatgcaggccctgccacctaga | |
| 247 | EVQLVESGGGLVKPGGSLRLSCAASGFTFSDYYMSWIRQAPGKGLEW | anti-BMCA CAR |
| | VSYISSSGSTIYYADSVKGRFTISRDNAKNSLYLQMNSLRAEDTAVY | |
| | YCAKVDGDYTEDYWGQGTLVTVSSGGGGSGGGGSGGGGSQSALTQPA | |
| | SVSGSPGQSITISCTGSSSDVGKYNLVSWYQQPPGKAPKLIIYDVNK | |
| | RPSGVSNRFSGSKSGNTATLTISGLQGDDEADYYCSSYGGSRSYVFG | |
| | TGTKVTVLESKYGPPCPPCPAPPVAGPSVFLFPPKPKDTLMISRTPE | |
| | VTCVVVDVSQEDPEVQFNWYVDGVEVHNAKTKPREEQFQSTYRVVSV | |
| | LTVLHQDWLNGKEYKCKVSNKGLPSSIEKTISKAKGQPREPQVYTLP | |
| | PSQEEMTKNQVSLTCLVKGFYPSDIAVEWESNGQPENNYKTTPPVLD | |
| | SDGSFFLYSRLTVDKSRWQEGNVFSCSVMHEALHNHYTQKSLSLSLG | |
| | KMFWVLVVVGGVLACYSLLVTVAFIIFWVKRGRKKLLYIFKQPFMRP | |
| | VQTTQEEDGCSCRFPEEEEGGCELRVKFSRSADAPAYQQGQNQLYNE | |
| | LNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 248 | EVQLVQSGGGLVQPGRSLRLSCTASGFTFGDYAMSWFKQAPGKGLEW | anti-BMCA CAR |
| | VGFIRSKAYGGTTEYAASVKGRFTISRDDSKSIAYLQMNSLKTEDTA | |
| | VYYCAAWSAPTDYWGQGTLVTVSSGGGGSGGGGSGGGGSDIQMTQSP | |
| | AFLSASVGDRVTVTCRASQGISNYLAWYQQKPGNAPRLLIYSASTLQ | |
| | SGVPSRFRGTGYGTEFSLTIDSLQPEDFATYYCQQSYTSRQTFGPGT | |
| | RLDIKESKYGPPCPPCPAPPVAGPSVFLFPPKPKDTLMISRTPEVTC | |
| | VVVDVSQEDPEVQFNWYVDGVEVHNAKTKPREEQFQSTYRVVSVLTV | |
| | LHQDWLNGKEYKCKVSNKGLPSSIEKTISKAKGQPREPQVYTLPPSQ | |
| | EEMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGKMF | |
| | WVLVVVGGVLACYSLLVTVAFIIFWVKRGRKKLLYIFKQPFMRPVQT | |
| | TQEEDGCSCRFPEEEEGGCELRVKFSRSADAPAYQQGQNQLYNELNL | |
| | GRREEYDVLDKRRGRDPEMGGKPRRKNPQEGLYNELQKDKMAEAYSE | |
| | IGMKGERRRGKGHDGLYQGLSTATKDTYDALHMQALPPR | |
| 249 | EVQLVESGGGLVKPGGSLRLSCAASGFTFSDYYMSWIRQAPGKGLEW | anti-BMCA CAR |
| | VSYISSSGSTIYYADSVKGRFTISRDNAKNSLYLQMNSLRAEDTAVY | |
| | YCAKVDGPPSFDIWGQGTMVTVSSGGGGSGGGGSGGGGSSYVLTQPP | |
| | SVSVAPGQTARITCGANNIGSKSVHWYQQKPGQAPMLVVYDDDDRPS | |
| | GIPERFSGSNSGNTATLTISGVEAGDEADYFCHLWDRSRDHYVFGTG | |
| | TKLTVLESKYGPPCPPCPAPPVAGPSVFLFPPKPKDTLMISRTPEVT | |
| | CVVVDVSQEDPEVQFNWYVDGVEVHNAKTKPREEQFQSTYRVVSVLT | |
| | VLHQDWLNGKEYKCKVSNKGLPSSIEKTISKAKGQPREPQVYTLPPS | |
| | QEEMTKNQVSLTCLVKGFYPSDIAVEWESNGQPENNYKTTPPVLDSD | |
| | GSFFLYSRLTVDKSRWQEGNVFSCSVMHEALHNHYTQKSLSLSLGKM | |
| | FWVLVVVGGVLACYSLLVTVAFIIFWVKRGRKKLLYIFKQPFMRPVQ | |
| | TTQEEDGCSCRFPEEEEGGCELRVKFSRSADAPAYQQGQNQLYNELN | |
| | LGRREEYDVLDKRRGRDPEMGGKPRRKNPQEGLYNELQKDKMAEAYS | |
| | EIGMKGERRRGKGHDGLYQGLSTATKDTYDALHMQALPPR | |
| 250 | SYELTQPPSASGTPGQRVTMSCSGTSSNIGSHSVNWYQQLPGTAPKL | anti-BMCA CAR |
| | LIYTNNQRPSGVPDRFSGSKSGTSASLAISGLQSEDEADYYCAAWDG | |
| | SLNGLVFGGGTKLTVLGSRGGGGSGGGGSGGGGSLEMAEVQLVQSGA | |
| | EVKKPGESLKISCKGSGYSFTSYWIGWVRQMPGKGLEWMGIIYPGDS | |
| | DTRYSPSFQGHVTISADKSISTAYLQWSSLKASDTAMYYCARYSGSF | |
| | DNWGQGTLVTVSSESKYGPPCPPCPAPPVAGPSVFLFPPKPKDTLMI | |
| | SRTPEVTCVVVDVSQEDPEVQFNWYVDGVEVHNAKTKPREEQFQSTY | |
| | RVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTT | |
| | PPVLDSDGSFFLYSRLTVDKSRWQEGNVFSCSVMHEALHNHYTQKSL | |
| | SLSLGKMFWVLVVVGGVLACYSLLVTVAFIIFWVKRGRKKLLYIFKQ | |
| | PFMRPVQTTQEEDGCSCRFPEEEEGGCELRVKFSRSADAPAYQQGQN | |
| | QLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR | |
| 251 | QSALTQPASVSASPGQSIAISCTGTSSDVGWYQQHPGKAPKLMIYED | anti-BMCA CAR |
| | SKRPSGVSNRFSGSKSGNTASLTISGLQAEDEADYYCSSNTRSSTLV | |
| | FGGGTKLTVLGSRGGGGSGGGGSGGGGSLEMAEVQLVQSGAEMKKPG | |
| | ASLKLSCKASGYTFIDYYVYWMRQAPGQGLESMGWINPNSGGTNYAQ | |
| | KFQGRVTMTRDTSISTAYMELSRLRSDDTAMYYCARSQRDGYMDYWG | |
| | QGTLVTVSSESKYGPPCPPCPAPPVAGPSVFLFPPKPKDTLMISRTP | |
| | EVTCVVVDVSQEDPEVQFNWYVDGVEVHNAKTKPREEQFQSTYRVVS | |
| | VLTVLHQDWLNGKEYKCKVSNKGLPSSIEKTISKAKGQPREPQVYTL | |
| | PPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSL | |
| | GKMFWVLVVVGGVLACYSLLVTVAFIIFWVKRGRKKLLYIFKQPFMR | |
| | PVQTTQEEDGCSCRFPEEEEGGCELRVKFSRSADAPAYQQGQNQLYN | |
| | ELNLGRREEYDVLDKRRGRDPEMGGKPRRKNPQEGLYNELQKDKMAE | |
| | AYSEIGMKGERRRGKGHDGLYQGLSTATKDTYDALHMQALPPR | |
| 252 | QSALTQPASVSASPGQSIAISCTGTSSDVGWYQQHPGKAPKLMIYED | anti-BMCA CAR |
| | SKRPSGVSNRFSGSKSGNTASLTISGLQAEDEADYYCSSNTRSSTLV | |
| | FGGGTKLTVLGSRGGGGSGGGGSGGGGSLEMAEVQLVQSGAEMKKPG | |
| | ASLKLSCKASGYTFIDYYVYWMRQAPGQGLESMGWINPNSGGTNYAQ | |
| | KFQGRVTMTRDTSISTAYMELSRLRSDDTAMYYCARSQRDGYMDYWG | |
| | QGTLVTVSSESKYGPPCPPCPAPPVAGPSVFLFPPKPKDTLMISRTP | |
| | EVTCVVVDVSQEDPEVQFNWYVDGVEVHNAKTKPREEQFQSTYRVVS | |
| | VLTVLHQDWLNGKEYKCKVSNKGLPSSIEKTISKAKGQPREPQVYTL | |
| | PPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSL | |
| | GKMFWVLVVVGGVLACYSLLVTVAFIIFWVRSKRSRLLHSDYMNMTP | |
| | RRPGPTRKHYQPYAPPRDFAAYRSRVKFSRSADAPAYQQGQNQLYNE | |
| | LNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| 253 | ACTGCATCGAGTCCACTGGAGA | GPRC5D CAR |
| | | forward primer |
| 254 | GGATCTTTCGCATGAGGAAGAG | GPRC5D CAR |
| | | reverse primer |
| 255 | AGCATCCCCCAAAGTTCAC | housekeeping gene |
| | | β-actin forward |
| | | primer |
| 256 | AAGGGACTTCCTGTAACAACG | housekeeping gene |
| | | β-actin reverse |
| | | primer |
| 257 | GAAGTGCAGCTGGTTGAAAGCGGCGGAGCCTTTGTTCAGCCTGGCGG | GPRC5D-200 |
| | ATCTCTGAGACTGAGCTGTGCCGCCAGCGGCTTCACCTTTAGCAGCT | VH/VL (nt) |
| | ACGCCATGACCTGGGTCCGACAGGCTCCTGGCAAAGGCCTTGAATGG | |
| | GTGTCCACCATCAGCGGCAGAGGCAGAAGCACCTTCTACGCCGATAG | |
| | CGTGAAGGGCAGATTCACCATCTCCAGAGACAACAGCAAGAACACCC | |
| | TGTACCTGCAGATGAACAGCCTGAGAGCCGAGGACACCGCCGTGTAC | |
| | TACTGCGCCAGATATTATCACGCTGGCGCCTTCGATCTGTGGGGCCA | |
| | GGGAACACTGGTCACAGTGTCTAGTGGAAGCAGAGGCGGCGGAGGAT | |
| | CTGGCGGAGGCGGTAGCGGTGGTGGTGGATCTCTTGAAATGGCCCAG | |
| | AGCGTGGTCACACAGCCTGCCTCTGTTTCTGGCTCTCCTGGCCAGAG | |
| | CATCACAATCAGCTGTACCGGCACCAGCTCTGACGTCGGCGGCTACA | |
| | ATTACGTGTCCTGGTATCAGCAGCACCCCGGCAAGGCCCCTAAGCTG | |
| | ATGATCTACGACGTGTCCAAGAGGCCCAGCGGCGTGTCCAATAGATT | |
| | CAGCGGCTCCAAGAGCGGCAACACCGCCAGCCTTACAATCAGCGGAC | |
| | TGCAGGCCGAGGACGAGGCCGATTACTACTGCAGCAGCTACACCAGC | |
| | AGCAGCACACTGGTTTTTGGAGGCGGCACCAAGCTGACCGTGCTT | |
| 258 | CAGTCTGTGGTTACACAGCCTGCCAGCGTGTCCGGATCTCCTGGCCA | GPRC5D-200- |
| | GAGCATCACCATCAGCTGTACCGGCACCAGCTCTGATGTCGGCGGCT | VL/VH (nt) |
| | ACAATTACGTGTCCTGGTATCAGCAGCACCCCGGCAAGGCCCCTAAG | |
| | CTGATGATCTACGACGTGTCCAAGAGGCCCAGCGGCGTGTCCAATAG | |
| | ATTCAGCGGCAGCAAGAGCGGCAACACCGCCAGCCTGACAATTAGCG | |
| | GACTGCAGGCCGAGGACGAGGCCGATTACTACTGTAGCAGCTACACC | |
| | AGCAGCAGCACACTGGTGTTTGGCGGAGGCACCAAGCTGACAGTGCT | |
| | TGGAAGTAGAGGCGGCGGAGGAAGCGGAGGCGGAGGATCTGGTGGTG | |
| | GTGGATCTCTGGAAATGGCCGAGGTGCAGCTGGTGGAATCTGGCGGA | |
| | GCTTTTGTTCAGCCTGGCGGCAGCCTGAGACTGTCTTGTGCTGCCAG | |
| | CGGCTTCACCTTCAGCAGCTACGCCATGACCTGGGTCCGACAGGCTC | |
| | CTGGCAAAGGCCTTGAATGGGTGTCCACCATCTCCGGCAGAGGCAGA | |
| | AGCACCTTCTACGCCGATAGCGTGAAGGGCAGATTCACAATCAGCCG | |
| | GGACAACAGCAAGAACACCCTGTACCTGCAGATGAACTCCCTGAGAG | |
| | CCGAGGACACCGCCGTGTACTACTGCGCCAGATATTATCACGCTGGC | |
| | GCCTTCGATCTGTGGGGCCAGGGAACACTGGTCACCGTTAGTTCT | |
| 259 | CAAATGCAGCTGGTTCAGTCTGGCGCCGAAGTGAAGAAACCTGGCGC | GPRC5D-201 |
| | CTCTGTGAAGGTGTCCTGCAAGGCCAGCGGCTACACCTTCAATAGAT | VH/VL (nt) |
| | ACGCCATCACCTGGGTCCGACAGGCCCCTGGACAAGGACTTGAATGG | |
| | ATGGGCTGGATCAGCGCCTACAACGGCAATAGCCACTACGCCCAGAA | |
| | ACTGCAGGGCAGAGTGACCATGACCACCGATACCTCTACCGGCACCG | |
| | CCTACATGGAACTGCGGAGACTGAGAAGCGACGACACCGCCGTGTAC | |
| | TACTGTGCCAGAATGGCCTACGATAGCTGGGGCCAGGGAACCCTGGT | |
| | TACAGTGTCTAGTGGAAGCAGAGGCGGCGGAGGATCTGGCGGAGGTG | |
| | GTAGTGGCGGAGGCGGATCTCTTGAAATGGCCCAGTCTGTGCTGACC | |
| | CAGCCTGCCTCTGTTTCTGGCTCTCCTGGCCAAAGCCTGACCATCTC | |
| | TTGTACCGGCACAAGCAACGACGTGGGAGCCTACAAATACGTGTCCT | |
| | GGTATCAGCAGTACCCCGGCAAGGCCCCTAAGCTGATCCTGTACGAC | |
| | GTGTTCAAGAGGCCCAGCGGCGTGTCCAATAGATTCAGCGGCAGCAA | |
| | GTCCGACAACACCGCCAGCCTGACAATCAGCGGACTGCAGGCCGAAG | |
| | ATGAGGCCGACTACTACTGCTTCAGCCTGACCTCCAGCAACACCTAC | |
| | GTGTTCGGCACCGGCACCAAAGTGACAGTGCTT | |
| 260 | CAATCTGTGCTGACACAGCCTGCCAGCGTGTCCGGATCTCCTGGACA | GPRC5D-201 VL- |
| | GAGCCTGACCATCAGCTGTACCGGCACCAGCAATGACGTGGGCGCCT | VH (nt) |
| | ACAAATACGTGTCCTGGTATCAGCAGTACCCCGGCAAGGCCCCTAAG | |
| | CTGATCCTGTACGACGTGTTCAAGAGGCCCAGCGGCGTGTCCAATAG | |
| | ATTCAGCGGCAGCAAGAGCGACAACACCGCCAGCCTGACAATTAGCG | |
| | GACTGCAGGCCGAGGACGAGGCCGACTACTACTGTTTTAGCCTGACC | |
| | TCCAGCAACACCTACGTGTTCGGCACCGGCACAAAAGTGACAGTGCT | |
| | GGGAAGTAGAGGCGGCGGAGGATCTGGCGGAGGTGGAAGTGGCGGAG | |
| | GCGGATCTCTTGAAATGGCCCAGATGCAGCTGGTGCAGTCTGGCGCC | |
| | GAAGTGAAAAAACCTGGCGCCTCCGTGAAGGTGTCCTGCAAGGCTTC | |
| | TGGCTACACCTTCAACAGATACGCCATCACCTGGGTCCGACAGGCCC | |
| | CTGGACAAGGACTTGAATGGATGGGCTGGATCTCCGCCTACAACGGC | |
| | AATAGCCACTACGCCCAGAAACTGCAGGGCAGAGTGACCATGACCAC | |
| | CGATACCTCTACCGGCACAGCCTACATGGAACTGCGGAGACTGAGAT | |
| | CCGACGACACCGCCGTGTACTACTGTGCCAGAATGGCCTACGATAGC | |
| | TGGGGCCAGGGAACCCTGGTTACCGTTTCTTCT | |
| 261 | GAAGTGCAGCTGGTTGAATCTGGCGGCGGACTGGTTAAGCCTGGCGG | GPRC5D-202 |
| | ATCTCTGAGACTGAGCTGTGCCGCCAGCGGCTTCACCTTCAGCGACT | VH/VL (nt) |
| | ACTACATGAGCTGGATCAGACAGGCCCCTGGCAAAGGCCTGGAATGG | |
| | GTGTCCTACATCAGCAGCTCTGGCAGCACCATCTACTACGCCGACAG | |
| | CGTGAAGGGCAGATTCACCATCAGCCGGGACAACGCCAAGAACAGCC | |
| | TGTACCTGCAGATGAACTCCCTGAGAGCCGAGGACACCGCCGTGTAC | |
| | TATTGTGCCAGAGGCTACGGCAAGGCCTACGATCAATGGGGCCAGGG | |
| | CACACTGGTCACAGTGTCTAGTGGAAGTAGAGGCGGCGGAGGATCTG | |
| | GCGGAGGTGGAAGTGGCGGAGGCGGTTCTCTTGAAATGGCCCAGTCT | |
| | GTGCTGACCCAGCCTCCTTCTGCTTCTGGCACACCTGGCCAGAGAGT | |
| | GACCATCAGCTGTAGCGGCAGCAGAAGCAACGTCGGCGGCAACTACG | |
| | TGTTCTGGTATCAGCAGGTCCCAGGCGCCACACCTAAACTGCTGATC | |
| | TACAGAAGCAATCAGCGGCCCAGCGGCGTGCCCGATAGATTTGCCGG | |
| | ATCTAAGTCTGGCAGCTCCGCCAGCCTGGCCATTTCTGGACTGAGAT | |
| | CTGAGGACGAGGCCGATTACTACTGCGCCACCTGGGATGATAGCCTG | |
| | AGCGGCTTTGTGTTTGGCACCGGCACCAAAGTGACCGTGCTT | |
| 262 | CAATCTGTGCTGACACAGCCTCCTAGCGCCTCTGGAACACCTGGCCA | GPRC5D-202 |
| | GAGAGTGACCATCAGCTGTAGCGGCAGCAGAAGCAACGTCGGCGGCA | VL/VH (nt) |
| | ACTACGTGTTCTGGTATCAGCAGGTCCCAGGCGCCACACCTAAGCTG | |
| | CTGATCTACAGAAGCAATCAGCGGCCTAGCGGCGTGCCCGATAGATT | |
| | TGCCGGAAGCAAGAGCGGCAGCTCTGCCAGCCTTGCCATCTCTGGAC | |
| | TGAGAAGCGAGGACGAGGCCGACTACTACTGTGCCACCTGGGATGAT | |
| | AGCCTGAGCGGCTTCGTGTTTGGCACCGGCACCAAAGTGACAGTGCT | |
| | GGGAAGTAGAGGCGGCGGAGGATCTGGCGGAGGTGGAAGTGGCGGAG | |
| | GCGGATCTCTTGAAATGGCCGAGGTGCAGCTGGTGGAATCTGGTGGC | |
| | GGACTTGTGAAGCCTGGCGGCTCTCTGAGACTGTCTTGTGCCGCCAG | |
| | CGGCTTCACCTTCAGCGATTACTACATGAGCTGGATCAGACAGGCCC | |
| | CTGGCAAAGGCCTGGAATGGGTGTCCTACATCAGCTCCAGCGGCTCT | |
| | ACCATCTACTACGCCGACAGCGTGAAGGGCAGATTCACCATCAGCCG | |
| | GGACAACGCCAAGAACAGCCTGTACCTGCAGATGAACTCCCTGAGAG | |
| | CCGAGGACACCGCCGTGTACTATTGTGCCAGAGGCTACGGCAAGGCC | |
| | TACGATCAATGGGGCCAGGGCACACTGGTCACCGTTAGTTCT | |
| 263 | CAAGTTCAGCTGGTGGAATCTGGCGGCGGACTGGTTCATCCTGGCGG | GPRC5D-203 |
| | ATCTCTGAGACTGAGCTGTGCCGCCAGCGGCTTCACCTTTAGAAGCC | VH/VL (nt) |
| | ACAGCATGAACTGGGTCCGACAGGCCCCTGGCAAAGGCCTTGAATGG | |
| | GTGTCCAGCATCAGCAGCGACAGCACCTACACCTACTACGCCGACAG | |
| | CGTGAAGGGCAGATTCACCATCTCCAGAGACAACGCCAAGAACAGCC | |
| | TGTACCTGCAGATGAACTCCCTGAGAGCCGAGGACACCGCCGTGTAC | |
| | TACTGTGCTAGATCTGGCGGACAGTGGAAGTACTACGACTACTGGGG | |
| | CCAGGGCACCCTGGTCACAGTTAGCTCTGGAAGTAGAGGCGGCGGAG | |
| | GAAGCGGAGGCGGAGGTTCTGGTGGCGGAGGATCTCTGGAAATGGCC | |
| | AGCAGCGAACTGACACAGGACCCTGCAGTGTCTGTGGCCCTGGGCCA | |
| | GACAGTGCGGATTACTTGTCAGGGCGACAGCCTGCGGAGCTACTATG | |
| | CCTCTTGGTATCAGCAGAAGCCCGGCCAGGCTCCTGTGCTGGTTATC | |
| | TACGGCAAGAACAACAGACCCAGCGGCATCCCCGATAGATTCAGCGG | |
| | AAGCAGCTCTGGCAATACCGCCAGCCTGACAATTACTGGCGCCCAGG | |
| | CCGAAGATGAGGCCGACTACTACTGCAACAGCAGAGACAGCTCCGGC | |
| | AATCCTCCTGTGGTTTTTGGCGGAGGCACCAAGCTGACAGTGCTC | |
| 264 | TCTTCTGAGCTGACCCAAGATCCTGCCGTGTCTGTGGCTCTGGGCCA | GPRC5D-203 |
| | GACAGTGCGGATTACCTGTCAGGGCGATAGCCTGAGAAGCTACTACG | VL/VH (nt) |
| | CCAGCTGGTATCAGCAGAAGCCTGGACAGGCTCCCGTGCTGGTCATC | |
| | TACGGCAAGAACAACAGACCCAGCGGCATCCCCGATAGATTCAGCGG | |
| | AAGCAGCTCTGGCAATACCGCCAGCCTGACAATTACTGGCGCCCAGG | |
| | CCGAAGATGAGGCCGACTACTACTGCAACAGCAGAGACAGCTCCGGC | |
| | AATCCTCCTGTGGTTTTTGGCGGAGGCACCAAGCTGACAGTGCTGGG | |
| | AAGTAGAGGTGGCGGAGGATCTGGCGGCGGAGGAAGCGGAGGCGGCG | |
| | GATCTCTTGAAATGGCTCAGGTGCAGCTGGTGGAATCAGGCGGTGGA | |
| | CTTGTTCACCCTGGCGGAAGCCTGAGACTGTCTTGTGCCGCCAGCGG | |
| | CTTCACCTTCAGATCCCACAGCATGAACTGGGTCCGACAGGCCCCTG | |
| | GCAAAGGCCTTGAATGGGTGTCCAGCATCAGCAGCGACAGCACCTAC | |
| | ACCTACTATGCCGACAGCGTGAAGGGCAGATTCACCATCTCCAGAGA | |
| | CAACGCCAAGAACAGCCTGTACCTGCAGATGAACTCCCTGAGAGCCG | |
| | AGGACACCGCCGTGTACTACTGTGCTAGAAGTGGCGGCCAGTGGAAG | |
| | TACTACGACTATTGGGGCCAGGGCACCCTGGTCACAGTTAGCTCT | |
| 265 | GAAGTGCAGCTGGTTGAATCTGGCGGCGGACTGGTTCAACCTGGCGG | GPRC5D-204 |
| | ATCTCTGAGACTGAGCTGTGCCGCCAGCGGCTTCACCTTCAGCAATT | VH/VL (nt) |
| | ACGCCATGAGCTGGGTCCGACAGGCCCCTGGAAAAGGCCTTGAATGG | |
| | GTGTCCGCCATCAGCGGCAGCGGCAATACCTACTACGCCGACTCTGT | |
| | GAAGGGCAGATTCACCATCAGCCGGGACAACAGCAAGAACACCCTGT | |
| | ACCTGCAGATGAACAGCCTGAGAGCCGAGGACACCGCCGTGTACTAT | |
| | TGTGCCAGAGGCAGCGTGCGGTACACCGATATTTGGGGCCAGGGCAC | |
| | ACTGGTCACAGTGTCTAGTGGAAGTAGAGGCGGCGGAGGATCTGGCG | |
| | GAGGTGGAAGTGGCGGAGGCGGTTCTCTGGAAATGGCCAACTTCATG | |
| | CTGACCCAGCCTCACAGCGTGTCCGAGTCTCCAGGCAAGACCGTGTC | |
| | CATCAGCTGCACCAGAACAAGCGGAGCCATTGCCGGCGCTTACGTGC | |
| | AGTGGTTCCAGCAGAGGCCTGGAAGCGCTCCTACCACCGTGATCTAC | |
| | GACGACAACAAGAGGCCTAGCGGCGTGCCCGATAGATTCAGCGGCTC | |
| | CATCGACAAGAGCAGCAACAGCGCCAGCCTGACAATCAGCGGCCTGA | |
| | AAACAGAGGACGAGGCCGACTACTACTGCCAGAGCTACGACTACGAC | |
| | AGCAGCAACGTGCTGTTTGGAGGCGGCACCAAGCTGACAGTGCTT | |
| 266 | AACTTCATGCTGACCCAGCCTCACAGCGTGTCCGAGTCTCCAGGCAA | GPRC5D-204 |
| | GACCGTGTCCATCAGCTGCACCAGAACAAGCGGAGCCATTGCCGGCG | VL/VH (nt) |
| | CTTACGTGCAGTGGTTCCAGCAGAGGCCTGGAAGCGCTCCTACCACC | |
| | GTGATCTACGACGACAACAAGAGGCCTAGCGGCGTGCCCGATAGATT | |
| | CAGCGGCAGCATCGACAAGAGCAGCAACAGCGCCAGCCTGACAATCA | |
| | GCGGCCTGAAAACAGAGGACGAGGCCGACTACTACTGCCAGAGCTAC | |
| | GACTACGACAGCAGCAACGTGCTGTTTGGCGGAGGCACCAAGCTGAC | |
| | AGTGCTTGGAAGTAGAGGCGGCGGAGGAAGCGGAGGCGGAGGATCTG | |
| | GTGGTGGTGGATCTCTGGAAATGGCCGAAGTGCAGCTGGTCGAGTCT | |
| | GGCGGAGGACTTGTTCAACCTGGCGGCAGCCTGAGACTGTCTTGTGC | |
| | CGCTTCCGGCTTCACCTTCAGCAACTACGCCATGTCCTGGGTCCGAC | |
| | AGGCCCCTGGAAAAGGACTGGAATGGGTGTCCGCCATCAGCGGCTCT | |
| | GGCAATACCTACTACGCCGACAGCGTGAAGGGCAGATTCACCATCAG | |
| | CCGGGACAACAGCAAGAACACCCTGTACCTGCAGATGAACTCCCTGA | |
| | GAGCCGAGGACACCGCCGTGTACTATTGTGCCAGAGGCAGCGTGCGG | |
| | TACACCGATATTTGGGGCCAGGGCACACTGGTCACCGTGTCATCT | |
| 267 | GAAGTGCAGCTGGTTGAATCTGGCGGCGGACTGATTCAGCCTGGCGG | GPRC5D-205 |
| | ATCTCTGAGACTGAGCTGTGCCGCCAGCGGCTTCACCTTCAGCAACT | VH/VL (nt) |
| | ACGCCATGAACTGGGTCCGACAGGCCCCTGGCAAAGGCCTTGAATGG | |
| | GTGTCCACCATCAACGGCAGAGGCAGCAGCACCATCTACGCCGATTC | |
| | TGTGAAGGGCAGATTCACCATCAGCCGGGACAACAGCAAGAACACCC | |
| | TGTACCTGCAGATGAACAGCCTGAGAGCCGAGGACACCGCCACCTAT | |
| | TACTGTGCCAGATATATCAGCAGAGGCCTGGGCGATTCTTGGGGCCA | |
| | GGGAACACTGGTCACAGTGTCTAGTGGAAGTAGAGGCGGCGGAGGAT | |
| | CTGGCGGAGGTGGAAGTGGCGGAGGCGGTTCTCTTGAAATGGCCCAG | |
| | TCTGTGGTCACCCAGCCACCTAGCATGTCTGCCGCTCCAGGACAGCA | |
| | AGTGACCATCTCTTGTAGCGGCGGCAACAGCAACATCGAGCGGAACT | |
| | ACGTGTCCTGGTATCTGCAGCTGCCTGGCACAGCCCCTAAGCTGGTC | |
| | ATCTTCGACAACGACAGACGGCCCAGCGGCATCCCCGATAGATTTTC | |
| | TGGCAGCAAGAGCGGCACCAGCGCCACACTGGGAATTACAGGACTGC | |
| | AGACAGGCGACGAGGCCGACTACTATTGTGGCACCTGGGACAGCTCC | |
| | CTGAGAGGCTGGGTTTTCGGAGGCGGCACAAAGCTGACAGTGCTC | |
| 268 | CAGTCTGTGGTTACACAGCCTCCTAGCATGTCTGCCGCTCCCGGGCA | GPRC5D-205 |
| | GCAAGTGACCATCTCTTGTAGCGGCGGCAACAGCAACATCGAGCGGA | VL/VH (nt) |
| | ACTACGTGTCCTGGTATCTGCAGCTGCCTGGCACAGCCCCTAAGCTG | |
| | GTCATCTTCGACAACGACAGACGGCCCAGCGGCATCCCCGATAGATT | |
| | TTCTGGCAGCAAGAGCGGCACCAGCGCCACACTGGGAATTACAGGAC | |
| | TGCAGACAGGCGACGAGGCCGACTACTATTGTGGCACCTGGGACTCT | |
| | AGCCTGAGAGGCTGGGTTTTCGGCGGAGGCACAAAGCTGACAGTGCT | |
| | GGGAAGTAGAGGCGGCGGAGGATCTGGCGGAGGTGGAAGTGGCGGAG | |
| | GCGGATCTCTTGAAATGGCCGAGGTGCAGCTGGTGGAATCAGGCGGA | |
| | GGACTGATTCAGCCTGGCGGCTCTCTGAGACTGTCTTGTGCCGCCAG | |
| | CGGCTTCACCTTCAGCAACTACGCCATGAACTGGGTCCGACAGGCCC | |
| | CTGGCAAAGGCCTTGAATGGGTGTCCACCATCAACGGCAGAGGCAGC | |
| | AGCACCATCTACGCCGATTCTGTGAAGGGCAGATTCACCATCAGCCG | |
| | GGACAACAGCAAGAACACCCTGTACCTGCAGATGAACTCCCTGAGAG | |
| | CCGAGGACACCGCCACCTATTACTGTGCCAGATATATCAGCAGAGGC | |
| | CTGGGCGATTCTTGGGGCCAGGGAACACTGGTCACAGTCTCTAGT | |
| 269 | CAAGTTCAGCTGGTTCAGTCTGGCGCCGAAGTGAAGAAACCTGGCGC | GPRC5D-206 |
| | CTCTGTGAAGGTGTCCTGCAAGGCCAGCGGCTACACCTTTACCAGCT | VH/VL (nt) |
| | ACTACATGCACTGGGTCCGACAGGCCCCTGGACAAGGACTTGAGTGG | |
| | ATGGGCATCATCAACCCTAGCGGCGGCAGCACAAGATACGCCCAGAA | |
| | ATTCCAGGGCAGAGTGACCATGACCAGAGACACCAGCACCTCCACCG | |
| | TGTATATGGAACTGAGCAGCCTGCGGAGCGAGGACACAGCCGTGTAC | |
| | TATTGTGCCAGAGGCAGCAGCAGATGGGGCGGATGGACAGGCGATTA | |
| | TTGGGGCCAGGGAACCCTGGTCACAGTGTCTAGTGGAAGTAGAGGCG | |
| | GCGGAGGATCTGGCGGAGGTGGAAGTGGCGGAGGCGGATCTCTTGAA | |
| | ATGGCCCAGTCTGCCCTGACACAGCCTGCCTCTGTTTCTGGCTCTCC | |
| | TGGCCAGAGCATCACCATCAGCTGTACCGGCACCAGCTCTGATGTCG | |
| | GCGGCTACAATTTCGTGTCCTGGTATCAGCAGCACCCCGGCAAGGCC | |
| | CCTAAAGTGATGATCTACGACGTGTCCAAGCGGCCCAGCGGCATCAG | |
| | CAATAGATTCAGCGGCAGCAAGAGCGGCAACACCGCCAGCCTGACAA | |
| | TTAGCGGACTGCAGGTTGAGGACGAGGCCGAGTACTACTGCAGCAGC | |
| | TACACAAGCACCAGAACCGTGATCTTCGCTGGCGGCACCAAAGTGAC | |
| | AGTGCTC | |
| 270 | CAATCTGCTCTGACACAGCCTGCCAGCGTGTCCGGATCTCCTGGCCA | GPRC5D-206 |
| | GAGCATCACCATCAGCTGTACCGGCACCAGCTCTGATGTCGGCGGCT | VL/VH (nt) |
| | ACAATTTCGTGTCCTGGTATCAGCAGCACCCCGGCAAGGCCCCTAAA | |
| | GTGATGATCTACGACGTGTCCAAGCGGCCCAGCGGCATCAGCAATAG | |
| | ATTCAGCGGCAGCAAGAGCGGCAACACCGCCAGCCTGACAATTAGCG | |
| | GACTGCAGGTTGAGGACGAGGCCGAGTACTACTGCAGCAGCTACACC | |
| | AGCACCAGAACCGTGATCTTTGCTGGCGGCACCAAAGTGACAGTGCT | |
| | GGGAAGTAGAGGCGGCGGAGGATCTGGCGGAGGTGGAAGTGGCGGAG | |
| | GCGGATCTCTTGAAATGGCTCAGGTGCAGCTGGTGCAGTCTGGCGCC | |
| | GAAGTGAAAAAACCTGGCGCCTCCGTGAAGGTGTCCTGCAAGGCTTC | |
| | TGGCTACACCTTTACCAGCTACTACATGCACTGGGTCCGACAGGCCC | |
| | CTGGACAAGGACTTGAGTGGATGGGCATCATCAACCCTAGCGGCGGC | |
| | AGCACAAGATACGCCCAGAAATTCCAGGGCAGAGTGACCATGACCAG | |
| | AGACACCTCCACCAGCACAGTGTATATGGAACTGAGCAGCCTGCGGA | |
| | GCGAGGACACAGCCGTGTACTATTGTGCCAGAGGCAGCAGCAGATGG | |
| | GGCGGATGGACAGGCGATTATTGGGGCCAGGGAACCCTGGTCACAGT | |
| | GTCTAGC | |
| 271 | atggtgctgcagacccaggtgttcatcagcctgctgctgtggatctc | human IgG-kappa |
| | cggagcatacgga | signal sequence |
| | | (nt) |
| 272 | MVLQTQVFISLLLWISGAYG | human IgG-kappa |
| | | signal peptide(aa) |
| 273 | atggtgctgcagacccaggtgttcatcagcctgctgctgtggatctc | human IgG-kappa |
| | tggcgcctacggc | signal sequence |
| | | (nt) |
| 274 | atggtgctgcagacccaggtgttcatcagcctgctgctgtggatctc | human IgG-kappa |
| | tggcgcctatgga | signal sequence |
| | | (nt) |
| 275 | atggtgctgcagacacaggtgttcatctccctgctgctgtggatctc | human IgG-kappa |
| | tggagcatacgga | signal sequence |
| | | (nt) |
| 276 | atggtgctgcagacacaggtgttcatcagcctgctgctgtggatctc | human IgG-kappa |
| | cggagcatacgga | signal sequence |
| | | (nt) |
| 277 | atgcttctcctggtgacaagccttctgctctgtgagttaccacaccc | GMCSFR alpha |
| | agcattcctcctgatccca | chain signal |
| | | sequence |
| 278 | MLLLVTSLLLCELPHPAFLLIP | GMCSFR alpha |
| | | chain signal |
| | | peptide |
| 279 | MALPVTALLLPLALLLHA | CD8 alpha signal |
| | | peptide |
| 280 | MPLLLLLPLLWAGALA | CD33 signal |
| | | peptide |
| 281 | ASTKGPSVFPLAPCSRSTSESTAALGCLVKDYFPEPVTVSWNSGALT | Human IgG4 Fc |
| | SGVHTFPAVLQSSGLYSLSSVVTVPSSSLGTKTYTCNVDHKPSNTKV | (Uniprot P01861) |
| | DKRVESKYGPPCPSCPAPEFLGGPSVFLFPPKPKDTLMISRTPEVTC | |
| | VVVDVSQEDPEVQFNWYVDGVEVHNAKTKPREEQFNSTYRVVSVLTV | |
| | LHQDWLNGKEYKCKVSNKGLPSSIEKTISKAKGQPREPQVYTLPPSQ | |
| | EEMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| 282 | ASTKGPSVFPLAPCSRSTSESTAALGCLVKDYFPEPVTVSWNSGALT | Human IgG2 Fc |
| | SGVHTFPAVLQSSGLYSLSSVVTVPSSNFGTQTYTCNVDHKPSNTKV | (Uniprot P01859) |
| | DKTVERKCCVECPPCPAPPVAGPSVFLFPPKPKDTLMISRTPEVTCV | |
| | VVDVSHEDPEVQFNWYVDGVEVHNAKTKPREEQFNSTFRVVSVLTVV | |
| | HQDWLNGKEYKCKVSNKGLPAPIEKTISKTKGQPREPQVYTLPPSRE | |
| | EMTKNQVSLTCLVKGFYPSDISVEWESNGQPENNYKTTPPMLDSDGS | |
| | FFLYSKLTVDKSRWQQGNVFSCSVMHEALHNHYTQKSLSLSPGK | |
| 283 | GAGTCTAAATACGGACCGCCTTGTCCTCCTTGTCCAGCTCCTCCTGT | IgG4/IgG2 hinge- |
| | TGCCGGACCTTCCGTGTTCCTGTTTCCTCCAAAGCCTAAGGACACCC | IgG2/IgG4 CH2- |
| | TGATGATCAGCAGGACCCCTGAAGTGACCTGCGTGGTGGTGGATGTG | IgG4 CH3 spacer |
| | TCCCAAGAGGATCCCGAGGTGCAGTTCAATTGGTACGTGGACGGCGT | codon optimized |
| | GGAAGTGCACAACGCCAAGACCAAGCCTAGAGAGGAACAGTTCCAGA | only (nt) |
| | GCACCTACAGAGTGGTGTCCGTGCTGACAGTGCTGCACCAGGATTGG | |
| | CTGAACGGCAAAGAGTACAAGTGCAAGGTGTCCAACAAGGGCCTGCC | |
| | TAGCAGCATCGAGAAAACCATCTCCAAGGCCAAGGGCCAGCCAAGAG | |
| | AGCCCCAGGTTTACACACTGCCTCCAAGCCAAGAGGAAATGACCAAG | |
| | AATCAGGTGTCCCTGACATGCCTGGTCAAGGGCTTCTACCCCTCCGA | |
| | TATCGCCGTGGAATGGGAGAGCAATGGCCAGCCTGAGAACAACTACA | |
| | AGACCACACCTCCTGTGCTGGACAGCGACGGCAGTTTCTTCCTGTAT | |
| | AGTAGACTCACCGTGGATAAATCAAGATGGCAAGAGGGCAACGTGTT | |
| | CAGCTGCAGCGTGATGCACGAGGCCCTGCACAACCACTACACCCAGA | |
| | AAAGCCTGAGCCTGTCTCTGGGCAAA | |
| 284 | gaatctaagtacggaccgccttgtcctccttgtcccgctcctcctgt | Alternative |
| | tgccggaccttccgtgttcctgtttcctccaaagcctaaggacaccc | CO/SSE |
| | tgatgatcagcaggacccctgaagtgacctgcgtggtggtggatgtg | IgG4/IgG2 hinge- |
| | tcccaagaggatcccgaggtgcagttcaactggtatgtggacggcgt | IgG2/IgG4 CH2- |
| | ggaagtgcacaacgccaagaccaagcctagagaggaacagttccaga | IgG4 CH3 (nt) |
| | gcacctacagagtggtgtccgtgctgacagtgctgcaccaggattgg | |
| | ctgaacggcaaagagtacaagtgcaaggtgtccaacaagggcctgcc | |
| | tagcagcatcgagaaaaccatctccaaggccaagggccagccaagag | |
| | agccccaggtttacacactgcctccaagccaagaggaaatgaccaag | |
| | aatcaggtgtccctgacatgcctggtcaagggcttctacccctccga | |
| | tatcgccgtggaatgggagagcaatggccagcctgagaacaactaca | |
| | agaccacacctcctgtgctggacagcgacggcagtttcttcctgtat | |
| | agtagactcaccgtggataaatcaagatggcaagagggcaacgtgtt | |
| | cagctgcagcgtgatgcacgaggccctgcacaaccactacacccaga | |
| | aaagcctgagcctgtctctgggcaag | |
| 285 | gaatctaagtacggaccgccctgccccccttgccct | Spacer |
| | | (IgG4hinge) (nt) |
| 286 | ESKYGPPCPPCPAPEFLGGPSVFLFPPKPKDTLMISRTPEVTCVVVD | Hinge-CH2-CH3 |
| | VSQEDPEVQFNWYVDGVEVHNAKTKPREEQFNSTYRVVSVLTVLHQD | spacer (aa) |
| | WLNGKEYKCKVSNKGLPSSIEKTISKAKGQPREPQVYTLPPSQEEMT | |
| | KNQVSLTCLVKGFYPSDIAVEWESNGQPENNYKTTPPVLDSDGSFFL | |
| | YSRLTVDKSRWQEGNVFSCSVMHEALHNHYTQKSLSLSLGK | |
| 287 | attgaagttatgtatcctcctccttacctagacaatgagaagagcaa | CD28 ectodomain |
| | tggaaccattatccatgtgaaagggaaacacctttgtccaagtcccc | spacer (nt) |
| | tatttcccggaccttctaagccc | |
| 288 | IEVMYPPPYLDNEKSNGTIIHVKGKHLCPSPLFPGPSKP | CD28 ectodomain |
| | | spacer (aa) |
| 289 | ATGCCGCTGCTGCTACTGCTGCCCCTGCTGTGGGCAGGGGCTCTAGC | anti-GPRC5D |
| | TTCTTCTGAGCTGACCCAAGATCCTGCCGTGTCTGTGGCTCTGGGCC | CAR |
| | AGACAGTGCGGATTACCTGTCAGGGCGATAGCCTGAGAAGCTACTAC | |
| | GCCAGCTGGTATCAGCAGAAGCCTGGACAGGCTCCCGTGCTGGTCAT | |
| | CTACGGCAAGAACAACAGACCCAGCGGCATCCCCGATAGATTCAGCG | |
| | GAAGCAGCTCTGGCAATACCGCCAGCCTGACAATTACTGGCGCCCAG | |
| | GCCGAAGATGAGGCCGACTACTACTGCAACAGCAGAGACAGCTCCGG | |
| | CAATCCTCCTGTGGTTTTTGGCGGAGGCACCAAGCTGACAGTGCTGG | |
| | GAAGTAGAGGTGGCGGAGGATCTGGCGGCGGAGGAAGCGGAGGCGGC | |
| | GGATCTCTTGAAATGGCTCAGGTGCAGCTGGTGGAATCAGGCGGTGG | |
| | ACTTGTTCACCCTGGCGGAAGCCTGAGACTGTCTTGTGCCGCCAGCG | |
| | GCTTCACCTTCAGATCCCACAGCATGAACTGGGTCCGACAGGCCCCT | |
| | GGCAAAGGCCTTGAATGGGTGTCCAGCATCAGCAGCGACAGCACCTA | |
| | CACCTACTATGCCGACAGCGTGAAGGGCAGATTCACCATCTCCAGAG | |
| | ACAACGCCAAGAACAGCCTGTACCTGCAGATGAACTCCCTGAGAGCC | |
| | GAGGACACCGCCGTGTACTACTGTGCTAGAAGTGGCGGCCAGTGGAA | |
| | GTACTACGACTATTGGGGCCAGGGCACCCTGGTCACAGTTAGCTCTG | |
| | AGTCTAAATACGGACCGCCTTGTCCTCCTTGTCCCGCTCCTCCTGTT | |
| | GCCGGACCTTCCGTGTTCCTGTTTCCTCCAAAGCCTAAGGACACCCT | |
| | GATGATCAGCAGGACCCCTGAAGTGACCTGCGTGGTGGTGGATGTGT | |
| | CCCAAGAGGATCCCGAGGTGCAGTTCAACTGGTATGTGGACGGCGTG | |
| | GAAGTGCACAACGCCAAGACCAAGCCTAGAGAGGAACAGTTCCAGAG | |
| | CACCTACAGAGTGGTGTCCGTGCTGACAGTGCTGCACCAGGATTGGC | |
| | TGAACGGCAAAGAGTACAAGTGCAAGGTGTCCAACAAGGGCCTGCCT | |
| | AGCAGCATCGAGAAAACCATCTCCAAGGCCAAGGGCCAGCCAAGAGA | |
| | GCCCCAGGTTTACACACTGCCTCCAAGCCAAGAGGAAATGACCAAGA | |
| | ATCAGGTGTCCCTGACATGCCTGGTCAAGGGCTTCTACCCCTCCGAT | |
| | ATCGCCGTGGAATGGGAGAGCAATGGCCAGCCTGAGAACAACTACAA | |
| | GACCACACCTCCTGTGCTGGACAGCGACGGCAGTTTCTTCCTGTATA | |
| | GTAGACTCACCGTGGATAAATCAAGATGGCAAGAGGGCAACGTGTTC | |
| | AGCTGCAGCGTGATGCACGAGGCCCTGCACAACCACTACACCCAGAA | |
| | AAGCCTGAGCCTGTCTCTGGGCAAGATGTTCTGGGTGCTCGTGGTCG | |
| | TTGGCGGAGTGCTGGCCTGTTACAGCCTGCTGGTTACCGTGGCCTTC | |
| | ATCATCTTTTGGGTCAAGCGGGGCAGAAAGAAGCTGCTCTACATCTT | |
| | CAAGCAGCCCTTCATGCGGCCCGTGCAGACCACACAAGAGGAAGATG | |
| | GCTGCTCCTGCAGATTCCCCGAGGAAGAAGAAGGCGGCTGCGAGCTG | |
| | AGAGTGAAGTTCAGCAGATCCGCCGACGCTCCAGCCTATCAGCAGGG | |
| | CCAAAACCAGCTGTACAACGAGCTGAACCTGGGGAGAAGAGAAGAGT | |
| | ACGACGTGCTGGATAAGCGGAGAGGCAGAGATCCTGAAATGGGCGGC | |
| | AAGCCCAGACGGAAGAATCCTCAAGAGGGCCTGTATAATGAGCTGCA | |
| | GAAAGACAAGATGGCCGAGGCCTACAGCGAGATCGGAATGAAGGGCG | |
| | AGCGCAGAAGAGGCAAGGGACACGATGGACTGTACCAGGGCCTGAGC | |
| | ACCGCCACCAAGGATACCTATGACGCACTGCACATGCAGGCCCTGCC | |
| | ACCTAGA | |
| 290 | SSELTQDPAVSVALGQTVRITCQGDSLRSYYASWYQQKPGQAPVLVI | anti-GPRC5D |
| | YGKNNRPSGIPDRFSGSSSGNTASLTITGAQAEDEADYYCNSRDSSG | CAR |
| | NPPVVFGGGTKLTVLGSRGGGGSGGGGSGGGGSLEMAQVQLVESGGG | |
| | LVHPGGSLRLSCAASGFTFRSHSMNWVRQAPGKGLEWVSSISSDSTY | |
| | TYYADSVKGRFTISRDNAKNSLYLQMNSLRAEDTAVYYCARSGGQWK | |
| | YYDYWGQGTLVTVSSESKYGPPCPPCPAPPVAGPSVFLFPPKPKDTL | |
| | MISRTPEVTCVVVDVSQEDPEVQFNWYVDGVEVHNAKTKPREEQFQS | |
| | TYRVVSVLTVLHQDWLNGKEYKCKVSNKGLPSSIEKTISKAKGQPRE | |
| | PQVYTLPPSQEEMTKNQVSLTCLVKGFYPSDIAVEWESNGQPENNYK | |
| | TTPPVLDSDGSFFLYSRLTVDKSRWQEGNVFSCSVMHEALHNHYTQK | |
| | SLSLSLGKMFWVLVVVGGVLACYSLLVTVAFIIFWVKRGRKKLLYIF | |
| | KQPFMRPVQTTQEEDGCSCRFPEEEEGGCELRVKFSRSADAPAYQQG | |
| | QNQLYNELNLGRREEYDVLDKRRGRDPEMGGKPRRKNPQEGLYNELQ | |
| | KDKMAEAYSEIGMKGERRRGKGHDGLYQGLSTATKDTYDALHMQALP | |
| | PR | |
| 291 | CAATGGTCAGAGTCGACCTT | BCMA sgRNA |
| | | target sequence |
| 292 | GGGUGUGGACCUCACCAGAA | GPRC5D exon 1 |
| | | gRNA1 |
| 293 | GAGAAGAAAAUAGUCUCCAG | GPRC5D exon 1 |
| | | gRNA2 |
| 294 | GUGACUCUCAUCAUGACCAG | GPRC5D exon 1 |
| | | gRNA3 |
| 295 | GACGACCGGGUCGUCCCACU | GPRC5D exon 1 |
| | | gRNA4 |
| 296 | GAGAACCAGGAGCUCUCCAG | GPRC5D exon 1 |
| | | gRNA5 |
| 297 | SSYTSTRTVIFAG | CDRL3 |
| 298 | MVLQTQVFISLLLWISGAYGQSALTQPASVSASPGQSIAISCTGTSS | BCMA-GPRC5D |
| | DVGWYQQHPGKAPKLMIYEDSKRPSGVSNRFSGSKSGNTASLTISGL | CAR (aa) |
| | QAEDEADYYCSSNTRSSTLVFGGGTKLTVLGSRGGGGSGGGGSGGGG | |
| | SLEMAEVQLVQSGAEMKKPGASLKLSCKASGYTFIDYYVYWMRQAPG | |
| | QGLESMGWINPNSGGTNYAQKFQGRVTMTRDTSISTAYMELSRLRSD | |
| | DTAMYYCARSQRDGYMDYWGQGTLVTVSSESKYGPPCPPCPAPPVAG | |
| | PSVFLFPPKPKDTLMISRTPEVTCVVVDVSQEDPEVQFNWYVDGVEV | |
| | HNAKTKPREEQFQSTYRVVSVLTVLHQDWLNGKEYKCKVSNKGLPSS | |
| | IEKTISKAKGQPREPQVYTLPPSQEEMTKNQVSLTCLVKGFYPSDIA | |
| | VEWESNGQPENNYKTTPPVLDSDGSFFLYSRLTVDKSRWQEGNVFSC | |
| | SVMHEALHNHYTQKSLSLSLGKMFWVLVVVGGVLACYSLLVTVAFII | |
| | FWVKRGRKKLLYIFKQPFMRPVQTTQEEDGCSCRFPEEEEGGCELRV | |
| | KFSRSADAPAYQQGQNQLYNELNLGRREEYDVLDKRRGRDPEMGGKP | |
| | RRKNPQEGLYNELQKDKMAEAYSEIGMKGERRRGKGHDGLYQGLSTA | |
| | TKDTYDALHMQALPPRLEGGGEGRGSLLTCGDVEENPGPRMPLLLLL | |
| | PLLWAGALASSELTQDPAVSVALGQTVRITCQGDSLRSYYASWYQQK | |
| | PGQAPVLVIYGKNNRPSGIPDRFSGSSSGNTASLTITGAQAEDEADY | |
| | YCNSRDSSGNPPVVFGGGTKLTVLGSRGGGGSGGGGSGGGGSLEMAQ | |
| | VQLVESGGGLVHPGGSLRLSCAASGFTFRSHSMNWVRQAPGKGLEWV | |
| | SSISSDSTYTYYADSVKGRFTISRDNAKNSLYLQMNSLRAEDTAVYY | |
| | CARSGGQWKYYDYWGQGTLVTVSSESKYGPPCPPCPAPPVAGPSVFL | |
| | FPPKPKDTLMISRTPEVTCVVVDVSQEDPEVQFNWYVDGVEVHNAKT | |
| | KPREEQFQSTYRVVSVLTVLHQDWLNGKEYKCKVSNKGLPSSIEKTI | |
| | SKAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMHE | |
| | ALHNHYTQKSLSLSLGKMFWVLVVVGGVLACYSLLVTVAFIIFWVKR | |
| | GRKKLLYIFKQPFMRPVQTTQEEDGCSCRFPEEEEGGCELRVKFSRS | |
| | ADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTY | |
| | DALHMQALPPR | |
| 299 | ATGGTGCTGCAGACCCAGGTGTTCATCAGCCTGCTGCTGTGGATCTC | BCMA-GPRC5D |
| | TGGCGCCTATGGACAGTCTGCCCTGACACAGCCTGCCAGCGTTAGTG | CAR codon |
| | CTAGTCCCGGACAGTCTATCGCCATCAGCTGTACCGGCACCAGCTCT | diverged (nt) |
| | GACGTTGGCTGGTATCAGCAGCACCCTGGCAAGGCCCCTAAGCTGAT | |
| | GATCTACGAGGACAGCAAGAGGCCCAGCGGCGTGTCCAATAGATTCA | |
| | GCGGCAGCAAGAGCGGCAACACCGCCAGCCTGACAATTAGCGGACTG | |
| | CAGGCCGAGGACGAGGCCGATTACTACTGCAGCAGCAACACCCGGTC | |
| | CAGCACACTGGTTTTTGGCGGAGGCACCAAGCTGACAGTGCTGGGAT | |
| | CTAGAGGTGGCGGAGGATCTGGCGGCGGAGGAAGCGGAGGCGGCGGA | |
| | TCTCTTGAAATGGCTGAAGTGCAGCTGGTGCAGTCTGGCGCCGAGAT | |
| | GAAGAAACCTGGCGCCTCTCTGAAGCTGAGCTGCAAGGCCAGCGGCT | |
| | ACACCTTCATCGACTACTACGTGTACTGGATGCGGCAGGCCCCTGGA | |
| | CAGGGACTCGAATCTATGGGCTGGATCAACCCCAATAGCGGCGGCAC | |
| | CAATTACGCCCAGAAATTCCAGGGCAGAGTGACCATGACCAGAGACA | |
| | CCAGCATCAGCACCGCCTACATGGAACTGAGCCGGCTGAGATCCGAC | |
| | GACACCGCCATGTACTACTGCGCCAGATCTCAGCGCGACGGCTACAT | |
| | GGATTATTGGGGCCAGGGAACCCTGGTCACCGTGTCCAGCGAGTCTA | |
| | AATACGGACCGCCTTGTCCTCCTTGTCCCGCTCCTCCTGTTGCCGGA | |
| | CCTTCCGTGTTCCTGTTTCCTCCAAAGCCTAAGGACACCCTGATGAT | |
| | CAGCAGGACCCCTGAAGTGACCTGCGTGGTGGTGGATGTGTCCCAAG | |
| | AGGATCCCGAGGTGCAGTTCAACTGGTATGTGGACGGCGTGGAAGTG | |
| | CACAACGCCAAGACCAAGCCTAGAGAGGAACAGTTCCAGAGCACCTA | |
| | CAGAGTGGTGTCCGTGCTGACAGTGCTGCACCAGGATTGGCTGAACG | |
| | GCAAAGAGTACAAGTGCAAGGTGTCCAACAAGGGCCTGCCTAGCAGC | |
| | ATCGAGAAAACCATCTCCAAGGCCAAGGGCCAGCCAAGAGAGCCCCA | |
| | GGTTTACACACTGCCTCCAAGCCAAGAGGAAATGACCAAGAATCAGG | |
| | TGTCCCTGACATGCCTGGTCAAGGGCTTCTACCCCTCCGATATCGCC | |
| | GTGGAATGGGAGAGCAATGGCCAGCCTGAGAACAACTACAAGACCAC | |
| | ACCTCCTGTGCTGGACAGCGACGGCAGTTTCTTCCTGTATAGTAGAC | |
| | TCACCGTGGATAAATCAAGATGGCAAGAGGGCAACGTGTTCAGCTGC | |
| | AGCGTGATGCACGAGGCCCTGCACAACCACTACACCCAGAAAAGCCT | |
| | GAGCCTGTCTCTGGGCAAGATGTTCTGGGTGCTCGTGGTCGTTGGCG | |
| | GAGTGCTGGCCTGTTACAGCCTGCTGGTTACCGTGGCCTTCATCATC | |
| | TTTTGGGTCAAGCGGGGCAGAAAGAAGCTGCTCTACATCTTCAAGCA | |
| | GCCCTTCATGCGGCCCGTGCAGACCACACAAGAGGAAGATGGCTGCT | |
| | CCTGCAGATTCCCCGAGGAAGAAGAAGGCGGCTGCGAGCTGAGAGTG | |
| | AAGTTCAGCAGATCCGCCGACGCTCCAGCCTATCAGCAGGGCCAAAA | |
| | CCAGCTGTACAACGAGCTGAACCTGGGGAGAAGAGAAGAGTACGACG | |
| | TGCTGGATAAGCGGAGAGGCAGAGATCCTGAAATGGGCGGCAAGCCC | |
| | AGACGGAAGAATCCTCAAGAGGGCCTGTATAATGAGCTGCAGAAAGA | |
| | CAAGATGGCCGAGGCCTACAGCGAGATCGGAATGAAGGGCGAGCGCA | |
| | GAAGAGGCAAGGGACACGATGGACTGTACCAGGGCCTGAGCACCGCC | |
| | ACCAAGGATACCTATGACGCACTGCACATGCAGGCCCTGCCACCTAG | |
| | ACTTGAAGGTGGTGGCGAAGGCAGAGGCAGCCTGCTTACATGCGGAG | |
| | ATGTGGAAGAGAACCCCGGACCTAGAATGCCGCTGCTGCTACTGCTG | |
| | CCCCTGCTGTGGGCAGGGGCTCTAGCTTCTTCTGAGCTGACCCAAGA | |
| | TCCTGCCGTGTCTGTGGCTCTGGGCCAGACAGTGCGGATTACCTGTC | |
| | AGGGCGATAGCCTGAGAAGCTACTACGCCAGCTGGTATCAGCAGAAG | |
| | CCTGGACAGGCTCCCGTGCTGGTCATCTACGGCAAGAACAACAGACC | |
| | CAGCGGCATCCCCGATAGATTCAGCGGAAGCAGCTCTGGCAATACCG | |
| | CCTCCCTGACAATTACTGGCGCCCAGGCCGAAGATGAGGCCGACTAC | |
| | TACTGCAACAGCAGAGACAGCTCCGGCAATCCTCCTGTGGTGTTTGG | |
| | AGGCGGAACCAAACTGACCGTGCTGGGCAGCAGAGGTGGAGGTGGAA | |
| | GCGGCGGTGGAGGCTCCGGAGGAGGCGGAAGCCTTGAGATGGCACAG | |
| | GTGCAGCTGGTGGAATCAGGCGGTGGACTTGTTCACCCTGGCGGAAG | |
| | CCTGAGACTGTCTTGTGCCGCCAGCGGCTTCACCTTCCGGTCCCACA | |
| | GCATGAACTGGGTCCGACAGGCCCCTGGCAAAGGCCTTGAATGGGTG | |
| | TCCAGCATCAGCAGCGACAGCACCTACACCTACTATGCCGACAGCGT | |
| | GAAGGGCAGATTCACCATCTCCAGAGACAACGCCAAGAACAGCCTGT | |
| | ACCTGCAGATGAACTCCCTGAGAGCCGAGGACACCGCCGTGTACTAC | |
| | TGTGCTAGAAGTGGCGGCCAGTGGAAGTACTACGACTATTGGGGACA | |
| | GGGCACCCTGGTCACAGTTAGCTCTGAATCCAAATACGGTCCTCCAT | |
| | GCCCTCCATGCCCAGCTCCACCCGTTGCTGGACCAAGCGTGTTTCTG | |
| | TTCCCACCAAAGCCCAAGGATACACTCATGATTAGCAGAACCCCAGA | |
| | AGTGACATGTGTCGTCGTGGACGTTTCCCAAGAGGACCCTGAGGTCC | |
| | AGTTTAACTGGTATGTGGATGGAGTGGAGGTGCATAATGCTAAGACT | |
| | AAGCCAAGAGAAGAGCAGTTTCAGTCCACCTATAGAGTGGTCTCCGT | |
| | GCTCACCGTGCTGCATCAGGACTGGCTCAATGGCAAGGAATATAAGT | |
| | GTAAAGTCTCCAACAAAGGACTGCCATCCAGCATCGAAAAGACCATT | |
| | AGCAAGGCCAAAGGACAGCCTAGAGAGCCTCAGGTCTACACTCTGCC | |
| | TCCCTCCCAAGAAGAGATGACTAAGAACCAGGTCTCTCTGACCTGCC | |
| | TGGTGAAGGGATTCTACCCTTCCGACATTGCTGTGGAGTGGGAGTCC | |
| | AATGGACAGCCAGAGAATAACTATAAGACTACACCACCCGTGCTCGA | |
| | TAGCGACGGCTCATTCTTTCTGTACTCTCGGCTGACAGTGGACAAGA | |
| | GCAGATGGCAGGAGGGAAACGTCTTTAGCTGCTCCGTGATGCATGAG | |
| | GCTCTGCATAACCACTATACCCAGAAGTCTCTGTCCCTGAGCCTCGG | |
| | CAAAATGTTTTGGGTGCTGGTGGTGGTCGGAGGCGTGCTGGCTTGCT | |
| | ATTCCCTGCTGGTCACAGTGGCCTTCATTATCTTCTGGGTGAAACGG | |
| | GGAAGAAAGAAACTCCTGTATATATTCAAACAACCATTTATGAGACC | |
| | AGTACAAACTACTCAAGAAGAGGATGGCTGTAGCTGCCGGTTTCCCG | |
| | AAGAAGAGGAAGGAGGATGTGAACTGCGGGTGAAGTTCTCCAGAAGC | |
| | GCCGACGCCCCTGCCTACCAGCAGGGACAGAATCAGCTGTATAACGA | |
| | ACTGAACCTGGGCAGAAGGGAGGAATACGACGTCCTGGACAAGAGAC | |
| | GAGGCCGGGACCCCGAGATGGGCGGAAAGCCTCGGCGGAAGAACCCC | |
| | CAGGAAGGCCTGTACAACGAACTGCAGAAGGACAAGATGGCTGAAGC | |
| | TTACTCCGAGATCGGCATGAAAGGAGAGCGGAGGCGGGGCAAGGGCC | |
| | ACGACGGCCTGTATCAGGGCCTGTCCACCGCCACAAAGGATACCTAC | |
| | GATGCCCTCCATATGCAGGCTCTGCCTCCAAGA | |
| 300 | ATGGTGCTGCAGACCCAGGTGTTCATCAGCCTGCTGCTGTGGATCTC | BCMA-GPRC5D |
| | TGGCGCCTATGGACAGTCTGCCCTGACACAGCCTGCCAGCGTTAGTG | CAR original (nt) |
| | CTAGTCCCGGACAGTCTATCGCCATCAGCTGTACCGGCACCAGCTCT | |
| | GACGTTGGCTGGTATCAGCAGCACCCTGGCAAGGCCCCTAAGCTGAT | |
| | GATCTACGAGGACAGCAAGAGGCCCAGCGGCGTGTCCAATAGATTCA | |
| | GCGGCAGCAAGAGCGGCAACACCGCCAGCCTGACAATTAGCGGACTG | |
| | CAGGCCGAGGACGAGGCCGATTACTACTGCAGCAGCAACACCCGGTC | |
| | CAGCACACTGGTTTTTGGCGGAGGCACCAAGCTGACAGTGCTGGGAT | |
| | CTAGAGGTGGCGGAGGATCTGGCGGCGGAGGAAGCGGAGGCGGCGGA | |
| | TCTCTTGAAATGGCTGAAGTGCAGCTGGTGCAGTCTGGCGCCGAGAT | |
| | GAAGAAACCTGGCGCCTCTCTGAAGCTGAGCTGCAAGGCCAGCGGCT | |
| | ACACCTTCATCGACTACTACGTGTACTGGATGCGGCAGGCCCCTGGA | |
| | CAGGGACTCGAATCTATGGGCTGGATCAACCCCAATAGCGGCGGCAC | |
| | CAATTACGCCCAGAAATTCCAGGGCAGAGTGACCATGACCAGAGACA | |
| | CCAGCATCAGCACCGCCTACATGGAACTGAGCCGGCTGAGATCCGAC | |
| | GACACCGCCATGTACTACTGCGCCAGATCTCAGCGCGACGGCTACAT | |
| | GGATTATTGGGGCCAGGGAACCCTGGTCACCGTGTCCAGCGAGTCTA | |
| | AATACGGACCGCCTTGTCCTCCTTGTCCCGCTCCTCCTGTTGCCGGA | |
| | CCTTCCGTGTTCCTGTTTCCTCCAAAGCCTAAGGACACCCTGATGAT | |
| | CAGCAGGACCCCTGAAGTGACCTGCGTGGTGGTGGATGTGTCCCAAG | |
| | AGGATCCCGAGGTGCAGTTCAACTGGTATGTGGACGGCGTGGAAGTG | |
| | CACAACGCCAAGACCAAGCCTAGAGAGGAACAGTTCCAGAGCACCTA | |
| | CAGAGTGGTGTCCGTGCTGACAGTGCTGCACCAGGATTGGCTGAACG | |
| | GCAAAGAGTACAAGTGCAAGGTGTCCAACAAGGGCCTGCCTAGCAGC | |
| | ATCGAGAAAACCATCTCCAAGGCCAAGGGCCAGCCAAGAGAGCCCCA | |
| | GGTTTACACACTGCCTCCAAGCCAAGAGGAAATGACCAAGAATCAGG | |
| | TGTCCCTGACATGCCTGGTCAAGGGCTTCTACCCCTCCGATATCGCC | |
| | GTGGAATGGGAGAGCAATGGCCAGCCTGAGAACAACTACAAGACCAC | |
| | ACCTCCTGTGCTGGACAGCGACGGCAGTTTCTTCCTGTATAGTAGAC | |
| | TCACCGTGGATAAATCAAGATGGCAAGAGGGCAACGTGTTCAGCTGC | |
| | AGCGTGATGCACGAGGCCCTGCACAACCACTACACCCAGAAAAGCCT | |
| | GAGCCTGTCTCTGGGCAAGATGTTCTGGGTGCTCGTGGTCGTTGGCG | |
| | GAGTGCTGGCCTGTTACAGCCTGCTGGTTACCGTGGCCTTCATCATC | |
| | TTTTGGGTCAAGCGGGGCAGAAAGAAGCTGCTCTACATCTTCAAGCA | |
| | GCCCTTCATGCGGCCCGTGCAGACCACACAAGAGGAAGATGGCTGCT | |
| | CCTGCAGATTCCCCGAGGAAGAAGAAGGCGGCTGCGAGCTGAGAGTG | |
| | AAGTTCAGCAGATCCGCCGACGCTCCAGCCTATCAGCAGGGCCAAAA | |
| | CCAGCTGTACAACGAGCTGAACCTGGGGAGAAGAGAAGAGTACGACG | |
| | TGCTGGATAAGCGGAGAGGCAGAGATCCTGAAATGGGCGGCAAGCCC | |
| | AGACGGAAGAATCCTCAAGAGGGCCTGTATAATGAGCTGCAGAAAGA | |
| | CAAGATGGCCGAGGCCTACAGCGAGATCGGAATGAAGGGCGAGCGCA | |
| | GAAGAGGCAAGGGACACGATGGACTGTACCAGGGCCTGAGCACCGCC | |
| | ACCAAGGATACCTATGACGCACTGCACATGCAGGCCCTGCCACCTAG | |
| | ACTTGAAGGTGGTGGCGAAGGCAGAGGCAGCCTGCTTACATGCGGAG | |
| | ATGTGGAAGAGAACCCCGGACCTAGAATGCCGCTGCTGCTACTGCTG | |
| | CCCCTGCTGTGGGCAGGGGCTCTAGCTTCTTCTGAGCTGACCCAAGA | |
| | TCCTGCCGTGTCTGTGGCTCTGGGCCAGACAGTGCGGATTACCTGTC | |
| | AGGGCGATAGCCTGAGAAGCTACTACGCCAGCTGGTATCAGCAGAAG | |
| | CCTGGACAGGCTCCCGTGCTGGTCATCTACGGCAAGAACAACAGACC | |
| | CAGCGGCATCCCCGATAGATTCAGCGGAAGCAGCTCTGGCAATACCG | |
| | CCAGCCTGACAATTACTGGCGCCCAGGCCGAAGATGAGGCCGACTAC | |
| | TACTGCAACAGCAGAGACAGCTCCGGCAATCCTCCTGTGGTTTTTGG | |
| | CGGAGGCACCAAGCTGACAGTGCTGGGAAGTAGAGGTGGCGGAGGAT | |
| | CTGGCGGCGGAGGAAGCGGAGGCGGCGGATCTCTTGAAATGGCTCAG | |
| | GTGCAGCTGGTGGAATCAGGCGGTGGACTTGTTCACCCTGGCGGAAG | |
| | CCTGAGACTGTCTTGTGCCGCCAGCGGCTTCACCTTCAGATCCCACA | |
| | GCATGAACTGGGTCCGACAGGCCCCTGGCAAAGGCCTTGAATGGGTG | |
| | TCCAGCATCAGCAGCGACAGCACCTACACCTACTATGCCGACAGCGT | |
| | GAAGGGCAGATTCACCATCTCCAGAGACAACGCCAAGAACAGCCTGT | |
| | ACCTGCAGATGAACTCCCTGAGAGCCGAGGACACCGCCGTGTACTAC | |
| | TGTGCTAGAAGTGGCGGCCAGTGGAAGTACTACGACTATTGGGGCCA | |
| | GGGCACCCTGGTCACAGTTAGCTCTGAGTCTAAATACGGACCGCCTT | |
| | GTCCTCCTTGTCCCGCTCCTCCTGTTGCCGGACCTTCCGTGTTCCTG | |
| | TTTCCTCCAAAGCCTAAGGACACCCTGATGATCAGCAGGACCCCTGA | |
| | AGTGACCTGCGTGGTGGTGGATGTGTCCCAAGAGGATCCCGAGGTGC | |
| | AGTTCAACTGGTATGTGGACGGCGTGGAAGTGCACAACGCCAAGACC | |
| | AAGCCTAGAGAGGAACAGTTCCAGAGCACCTACAGAGTGGTGTCCGT | |
| | GCTGACAGTGCTGCACCAGGATTGGCTGAACGGCAAAGAGTACAAGT | |
| | GCAAGGTGTCCAACAAGGGCCTGCCTAGCAGCATCGAGAAAACCATC | |
| | TCCAAGGCCAAGGGCCAGCCAAGAGAGCCCCAGGTTTACACACTGCC | |
| | TCCAAGCCAAGAGGAAATGACCAAGAATCAGGTGTCCCTGACATGCC | |
| | TGGTCAAGGGCTTCTACCCCTCCGATATCGCCGTGGAATGGGAGAGC | |
| | AATGGCCAGCCTGAGAACAACTACAAGACCACACCTCCTGTGCTGGA | |
| | CAGCGACGGCAGTTTCTTCCTGTATAGTAGACTCACCGTGGATAAAT | |
| | CAAGATGGCAAGAGGGCAACGTGTTCAGCTGCAGCGTGATGCACGAG | |
| | GCCCTGCACAACCACTACACCCAGAAAAGCCTGAGCCTGTCTCTGGG | |
| | CAAGATGTTCTGGGTGCTCGTGGTCGTTGGCGGAGTGCTGGCCTGTT | |
| | ACAGCCTGCTGGTTACCGTGGCCTTCATCATCTTTTGGGTCAAGCGG | |
| | GGCAGAAAGAAGCTGCTCTACATCTTCAAGCAGCCCTTCATGCGGCC | |
| | CGTGCAGACCACACAAGAGGAAGATGGCTGCTCCTGCAGATTCCCCG | |
| | AGGAAGAAGAAGGCGGCTGCGAGCTGAGAGTGAAGTTCAGCAGATCC | |
| | GCCGACGCTCCAGCCTATCAGCAGGGCCAAAACCAGCTGTACAACGA | |
| | GCTGAACCTGGGGAGAAGAGAAGAGTACGACGTGCTGGATAAGCGGA | |
| | GAGGCAGAGATCCTGAAATGGGCGGCAAGCCCAGACGGAAGAATCCT | |
| | CAAGAGGGCCTGTATAATGAGCTGCAGAAAGACAAGATGGCCGAGGC | |
| | CTACAGCGAGATCGGAATGAAGGGCGAGCGCAGAAGAGGCAAGGGAC | |
| | ACGATGGACTGTACCAGGGCCTGAGCACCGCCACCAAGGATACCTAT | |
| | GACGCACTGCACATGCAGGCCCTGCCACCTAGA | |
| 301 | MPLLLLLPLLWAGALASSELTQDPAVSVALGQTVRITCQGDSLRSYY | GPRC5D-BCMA |
| | ASWYQQKPGQAPVLVIYGKNNRPSGIPDRFSGSSSGNTASLTITGAQ | CAR (aa) |
| | AEDEADYYCNSRDSSGNPPVVFGGGTKLTVLGSRGGGGSGGGGSGGG | |
| | GSLEMAQVQLVESGGGLVHPGGSLRLSCAASGFTFRSHSMNWVRQAP | |
| | GKGLEWVSSISSDSTYTYYADSVKGRFTISRDNAKNSLYLQMNSLRA | |
| | EDTAVYYCARSGGQWKYYDYWGQGTLVTVSSESKYGPPCPPCPAPPV | |
| | AGPSVFLFPPKPKDTLMISRTPEVTCVVVDVSQEDPEVQFNWYVDGV | |
| | EVHNAKTKPREEQFQSTYRVVSVLTVLHQDWLNGKEYKCKVSNKGLP | |
| | SSIEKTISKAKGQPREPQVYTLPPSQEEMTKNQVSLTCLVKGFYPSD | |
| | IAVEWESNGQPENNYKTTPPVLDSDGSFFLYSRLTVDKSRWQEGNVF | |
| | SCSVMHEALHNHYTQKSLSLSLGKMFWVLVVVGGVLACYSLLVTVAF | |
| | IIFWVKRGRKKLLYIFKQPFMRPVQTTQEEDGCSCRFPEEEEGGCEL | |
| | RVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLS | |
| | TATKDTYDALHMQALPPRLEGGGEGRGSLLTCGDVEENPGPRMVLQT | |
| | QVFISLLLWISGAYGQSALTQPASVSASPGQSIAISCTGTSSDVGWY | |
| | QQHPGKAPKLMIYEDSKRPSGVSNRFSGSKSGNTASLTISGLQAEDE | |
| | ADYYCSSNTRSSTLVFGGGTKLTVLGSRGGGGSGGGGSGGGGSLEMA | |
| | EVQLVQSGAEMKKPGASLKLSCKASGYTFIDYYVYWMRQAPGQGLES | |
| | MGWINPNSGGTNYAQKFQGRVTMTRDTSISTAYMELSRLRSDDTAMY | |
| | YCARSQRDGYMDYWGQGTLVTVSSESKYGPPCPPCPAPPVAGPSVFL | |
| | FPPKPKDTLMISRTPEVTCVVVDVSQEDPEVQFNWYVDGVEVHNAKT | |
| | KPREEQFQSTYRVVSVLTVLHQDWLNGKEYKCKVSNKGLPSSIEKTI | |
| | SKAKGQPREPQVYTLPPSQEEMTKNQVSLTCLVKGFYPSDIAVEWES | |
| | NGQPENNYKTTPPVLDSDGSFFLYSRLTVDKSRWQEGNVFSCSVMHE | |
| | ALHNHYTQKSLSLSLGKMFWVLVVVGGVLACYSLLVTVAFIIFWVKR | |
| | GRKKLLYIFKQPFMRPVQTTQEEDGCSCRFPEEEEGGCELRVKFSRS | |
| | ADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTY | |
| | DALHMQALPPR | |
| 302 | ATGCCGCTGCTGCTACTGCTGCCCCTGCTGTGGGCAGGGGCTCTAGC | GPRC5D-BCMA |
| | TTCTTCTGAGCTGACCCAAGATCCTGCCGTGTCTGTGGCTCTGGGCC | CAR codon |
| | AGACAGTGCGGATTACCTGTCAGGGCGATAGCCTGAGAAGCTACTAC | diverged (nt) |
| | GCCAGCTGGTATCAGCAGAAGCCTGGACAGGCTCCCGTGCTGGTCAT | |
| | CTACGGCAAGAACAACAGACCCAGCGGCATCCCCGATAGATTCAGCG | |
| | GAAGCAGCTCTGGCAATACCGCCTCCCTGACAATTACTGGCGCCCAG | |
| | GCCGAAGATGAGGCCGACTACTACTGCAACAGCAGAGACAGCTCCGG | |
| | CAATCCTCCTGTGGTGTTTGGAGGCGGAACCAAACTGACCGTGCTGG | |
| | GCAGCAGAGGTGGAGGTGGAAGCGGCGGTGGAGGCTCCGGAGGAGGC | |
| | GGAAGCCTTGAGATGGCACAGGTGCAGCTGGTGGAATCAGGCGGTGG | |
| | ACTTGTTCACCCTGGCGGAAGCCTGAGACTGTCTTGTGCCGCCAGCG | |
| | GCTTCACCTTCCGGTCCCACAGCATGAACTGGGTCCGACAGGCCCCT | |
| | GGCAAAGGCCTTGAATGGGTGTCCAGCATCAGCAGCGACAGCACCTA | |
| | CACCTACTATGCCGACAGCGTGAAGGGCAGATTCACCATCTCCAGAG | |
| | ACAACGCCAAGAACAGCCTGTACCTGCAGATGAACTCCCTGAGAGCC | |
| | GAGGACACCGCCGTGTACTACTGTGCTAGAAGTGGCGGCCAGTGGAA | |
| | GTACTACGACTATTGGGGACAGGGCACCCTGGTCACAGTTAGCTCTG | |
| | AATCCAAATACGGTCCTCCATGCCCTCCATGCCCAGCTCCACCCGTT | |
| | GCTGGACCAAGCGTGTTTCTGTTCCCACCAAAGCCCAAGGATACACT | |
| | CATGATTAGCAGAACCCCAGAAGTGACATGTGTCGTCGTGGACGTTT | |
| | CCCAAGAGGACCCTGAGGTCCAGTTTAACTGGTATGTGGATGGAGTG | |
| | GAGGTGCATAATGCTAAGACTAAGCCAAGAGAAGAGCAGTTTCAGTC | |
| | CACCTATAGAGTGGTCTCCGTGCTCACCGTGCTGCATCAGGACTGGC | |
| | TCAATGGCAAGGAATATAAGTGTAAAGTCTCCAACAAAGGACTGCCA | |
| | TCCAGCATCGAAAAGACCATTAGCAAGGCCAAAGGACAGCCTAGAGA | |
| | GCCTCAGGTCTACACTCTGCCTCCCTCCCAAGAAGAGATGACTAAGA | |
| | ACCAGGTCTCTCTGACCTGCCTGGTGAAGGGATTCTACCCTTCCGAC | |
| | ATTGCTGTGGAGTGGGAGTCCAATGGACAGCCAGAGAATAACTATAA | |
| | GACTACACCACCCGTGCTCGATAGCGACGGCTCATTCTTTCTGTACT | |
| | CTCGGCTGACAGTGGACAAGAGCAGATGGCAGGAGGGAAACGTCTTT | |
| | AGCTGCTCCGTGATGCATGAGGCTCTGCATAACCACTATACCCAGAA | |
| | GTCTCTGTCCCTGAGCCTCGGCAAAATGTTTTGGGTGCTGGTGGTGG | |
| | TCGGAGGCGTGCTGGCTTGCTATTCCCTGCTGGTCACAGTGGCCTTC | |
| | ATTATCTTCTGGGTGAAACGGGGAAGAAAGAAACTCCTGTATATATT | |
| | CAAACAACCATTTATGAGACCAGTACAAACTACTCAAGAAGAGGATG | |
| | GCTGTAGCTGCCGGTTTCCCGAAGAAGAGGAAGGAGGATGTGAACTG | |
| | CGGGTGAAGTTCTCCAGAAGCGCCGACGCCCCTGCCTACCAGCAGGG | |
| | ACAGAATCAGCTGTATAACGAACTGAACCTGGGCAGAAGGGAGGAAT | |
| | ACGACGTCCTGGACAAGAGACGAGGCCGGGACCCCGAGATGGGCGGA | |
| | AAGCCTCGGCGGAAGAACCCCCAGGAAGGCCTGTACAACGAACTGCA | |
| | GAAGGACAAGATGGCTGAAGCTTACTCCGAGATCGGCATGAAAGGAG | |
| | AGCGGAGGCGGGGCAAGGGCCACGACGGCCTGTATCAGGGCCTGTCC | |
| | ACCGCCACAAAGGATACCTACGATGCCCTCCATATGCAGGCTCTGCC | |
| | TCCAAGACTCGAGGGCGGCGGAGAGGGCAGAGGAAGTCTTCTAACAT | |
| | GCGGTGACGTGGAGGAGAATCCCGGCCCTAGGATGGTGCTGCAGACC | |
| | CAGGTGTTCATCAGCCTGCTGCTGTGGATCTCTGGCGCCTATGGACA | |
| | GTCTGCCCTGACACAGCCTGCCAGCGTTAGTGCTAGTCCCGGACAGT | |
| | CTATCGCCATCAGCTGTACCGGCACCAGCTCTGACGTTGGCTGGTAT | |
| | CAGCAGCACCCTGGCAAGGCCCCTAAGCTGATGATCTACGAGGACAG | |
| | CAAGAGGCCCAGCGGCGTGTCCAATAGATTCAGCGGCAGCAAGAGCG | |
| | GCAACACCGCCAGCCTGACAATTAGCGGACTGCAGGCCGAGGACGAG | |
| | GCCGATTACTACTGCAGCAGCAACACCCGGTCCAGCACACTGGTTTT | |
| | TGGCGGAGGCACCAAGCTGACAGTGCTGGGATCTAGAGGTGGCGGAG | |
| | GATCTGGCGGCGGAGGAAGCGGAGGCGGCGGATCTCTTGAAATGGCT | |
| | GAAGTGCAGCTGGTGCAGTCTGGCGCCGAGATGAAGAAACCTGGCGC | |
| | CTCTCTGAAGCTGAGCTGCAAGGCCAGCGGCTACACCTTCATCGACT | |
| | ACTACGTGTACTGGATGCGGCAGGCCCCTGGACAGGGACTCGAATCT | |
| | ATGGGCTGGATCAACCCCAATAGCGGCGGCACCAATTACGCCCAGAA | |
| | ATTCCAGGGCAGAGTGACCATGACCAGAGACACCAGCATCAGCACCG | |
| | CCTACATGGAACTGAGCCGGCTGAGATCCGACGACACCGCCATGTAC | |
| | TACTGCGCCAGATCTCAGCGCGACGGCTACATGGATTATTGGGGCCA | |
| | GGGAACCCTGGTCACCGTGTCCAGCGAGTCTAAATACGGACCGCCTT | |
| | GTCCTCCTTGTCCCGCTCCTCCTGTTGCCGGACCTTCCGTGTTCCTG | |
| | TTTCCTCCAAAGCCTAAGGACACCCTGATGATCAGCAGGACCCCTGA | |
| | AGTGACCTGCGTGGTGGTGGATGTGTCCCAAGAGGATCCCGAGGTGC | |
| | AGTTCAACTGGTATGTGGACGGCGTGGAAGTGCACAACGCCAAGACC | |
| | AAGCCTAGAGAGGAACAGTTCCAGAGCACCTACAGAGTGGTGTCCGT | |
| | GCTGACAGTGCTGCACCAGGATTGGCTGAACGGCAAAGAGTACAAGT | |
| | GCAAGGTGTCCAACAAGGGCCTGCCTAGCAGCATCGAGAAAACCATC | |
| | TCCAAGGCCAAGGGCCAGCCAAGAGAGCCCCAGGTTTACACACTGCC | |
| | TCCAAGCCAAGAGGAAATGACCAAGAATCAGGTGTCCCTGACATGCC | |
| | TGGTCAAGGGCTTCTACCCCTCCGATATCGCCGTGGAATGGGAGAGC | |
| | AATGGCCAGCCTGAGAACAACTACAAGACCACACCTCCTGTGCTGGA | |
| | CAGCGACGGCAGTTTCTTCCTGTATAGTAGACTCACCGTGGATAAAT | |
| | CAAGATGGCAAGAGGGCAACGTGTTCAGCTGCAGCGTGATGCACGAG | |
| | GCCCTGCACAACCACTACACCCAGAAAAGCCTGAGCCTGTCTCTGGG | |
| | CAAGATGTTCTGGGTGCTCGTGGTCGTTGGCGGAGTGCTGGCCTGTT | |
| | ACAGCCTGCTGGTTACCGTGGCCTTCATCATCTTTTGGGTCAAGCGG | |
| | GGCAGAAAGAAGCTGCTCTACATCTTCAAGCAGCCCTTCATGCGGCC | |
| | CGTGCAGACCACACAAGAGGAAGATGGCTGCTCCTGCAGATTCCCCG | |
| | AGGAAGAAGAAGGCGGCTGCGAGCTGAGAGTGAAGTTCAGCAGATCC | |
| | GCCGACGCTCCAGCCTATCAGCAGGGCCAAAACCAGCTGTACAACGA | |
| | GCTGAACCTGGGGAGAAGAGAAGAGTACGACGTGCTGGATAAGCGGA | |
| | GAGGCAGAGATCCTGAAATGGGCGGCAAGCCCAGACGGAAGAATCCT | |
| | CAAGAGGGCCTGTATAATGAGCTGCAGAAAGACAAGATGGCCGAGGC | |
| | CTACAGCGAGATCGGAATGAAGGGCGAGCGCAGAAGAGGCAAGGGAC | |
| | ACGATGGACTGTACCAGGGCCTGAGCACCGCCACCAAGGATACCTAT | |
| | GACGCACTGCACATGCAGGCCCTGCCACCTAGA | |
| 303 | ATGCCGCTGCTGCTACTGCTGCCCCTGCTGTGGGCAGGGGCTCTAGC | GPRC5D-BCMA |
| | TTCTTCTGAGCTGACCCAAGATCCTGCCGTGTCTGTGGCTCTGGGCC | CAR original (nt) |
| | AGACAGTGCGGATTACCTGTCAGGGCGATAGCCTGAGAAGCTACTAC | |
| | GCCAGCTGGTATCAGCAGAAGCCTGGACAGGCTCCCGTGCTGGTCAT | |
| | CTACGGCAAGAACAACAGACCCAGCGGCATCCCCGATAGATTCAGCG | |
| | GAAGCAGCTCTGGCAATACCGCCAGCCTGACAATTACTGGCGCCCAG | |
| | GCCGAAGATGAGGCCGACTACTACTGCAACAGCAGAGACAGCTCCGG | |
| | CAATCCTCCTGTGGTTTTTGGCGGAGGCACCAAGCTGACAGTGCTGG | |
| | GAAGTAGAGGTGGCGGAGGATCTGGCGGCGGAGGAAGCGGAGGCGGC | |
| | GGATCTCTTGAAATGGCTCAGGTGCAGCTGGTGGAATCAGGCGGTGG | |
| | ACTTGTTCACCCTGGCGGAAGCCTGAGACTGTCTTGTGCCGCCAGCG | |
| | GCTTCACCTTCAGATCCCACAGCATGAACTGGGTCCGACAGGCCCCT | |
| | GGCAAAGGCCTTGAATGGGTGTCCAGCATCAGCAGCGACAGCACCTA | |
| | CACCTACTATGCCGACAGCGTGAAGGGCAGATTCACCATCTCCAGAG | |
| | ACAACGCCAAGAACAGCCTGTACCTGCAGATGAACTCCCTGAGAGCC | |
| | GAGGACACCGCCGTGTACTACTGTGCTAGAAGTGGCGGCCAGTGGAA | |
| | GTACTACGACTATTGGGGCCAGGGCACCCTGGTCACAGTTAGCTCTG | |
| | AGTCTAAATACGGACCGCCTTGTCCTCCTTGTCCCGCTCCTCCTGTT | |
| | GCCGGACCTTCCGTGTTCCTGTTTCCTCCAAAGCCTAAGGACACCCT | |
| | GATGATCAGCAGGACCCCTGAAGTGACCTGCGTGGTGGTGGATGTGT | |
| | CCCAAGAGGATCCCGAGGTGCAGTTCAACTGGTATGTGGACGGCGTG | |
| | GAAGTGCACAACGCCAAGACCAAGCCTAGAGAGGAACAGTTCCAGAG | |
| | CACCTACAGAGTGGTGTCCGTGCTGACAGTGCTGCACCAGGATTGGC | |
| | TGAACGGCAAAGAGTACAAGTGCAAGGTGTCCAACAAGGGCCTGCCT | |
| | AGCAGCATCGAGAAAACCATCTCCAAGGCCAAGGGCCAGCCAAGAGA | |
| | GCCCCAGGTTTACACACTGCCTCCAAGCCAAGAGGAAATGACCAAGA | |
| | ATCAGGTGTCCCTGACATGCCTGGTCAAGGGCTTCTACCCCTCCGAT | |
| | ATCGCCGTGGAATGGGAGAGCAATGGCCAGCCTGAGAACAACTACAA | |
| | GACCACACCTCCTGTGCTGGACAGCGACGGCAGTTTCTTCCTGTATA | |
| | GTAGACTCACCGTGGATAAATCAAGATGGCAAGAGGGCAACGTGTTC | |
| | AGCTGCAGCGTGATGCACGAGGCCCTGCACAACCACTACACCCAGAA | |
| | AAGCCTGAGCCTGTCTCTGGGCAAGATGTTCTGGGTGCTCGTGGTCG | |
| | TTGGCGGAGTGCTGGCCTGTTACAGCCTGCTGGTTACCGTGGCCTTC | |
| | ATCATCTTTTGGGTCAAGCGGGGCAGAAAGAAGCTGCTCTACATCTT | |
| | CAAGCAGCCCTTCATGCGGCCCGTGCAGACCACACAAGAGGAAGATG | |
| | GCTGCTCCTGCAGATTCCCCGAGGAAGAAGAAGGCGGCTGCGAGCTG | |
| | AGAGTGAAGTTCAGCAGATCCGCCGACGCTCCAGCCTATCAGCAGGG | |
| | CCAAAACCAGCTGTACAACGAGCTGAACCTGGGGAGAAGAGAAGAGT | |
| | ACGACGTGCTGGATAAGCGGAGAGGCAGAGATCCTGAAATGGGCGGC | |
| | AAGCCCAGACGGAAGAATCCTCAAGAGGGCCTGTATAATGAGCTGCA | |
| | GAAAGACAAGATGGCCGAGGCCTACAGCGAGATCGGAATGAAGGGCG | |
| | AGCGCAGAAGAGGCAAGGGACACGATGGACTGTACCAGGGCCTGAGC | |
| | ACCGCCACCAAGGATACCTATGACGCACTGCACATGCAGGCCCTGCC | |
| | ACCTAGACTCGAGGGCGGCGGAGAGGGCAGAGGAAGTCTTCTAACAT | |
| | GCGGTGACGTGGAGGAGAATCCCGGCCCTAGGATGGTGCTGCAGACC | |
| | CAGGTGTTCATCAGCCTGCTGCTGTGGATCTCTGGCGCCTATGGACA | |
| | GTCTGCCCTGACACAGCCTGCCAGCGTTAGTGCTAGTCCCGGACAGT | |
| | CTATCGCCATCAGCTGTACCGGCACCAGCTCTGACGTTGGCTGGTAT | |
| | CAGCAGCACCCTGGCAAGGCCCCTAAGCTGATGATCTACGAGGACAG | |
| | CAAGAGGCCCAGCGGCGTGTCCAATAGATTCAGCGGCAGCAAGAGCG | |
| | GCAACACCGCCAGCCTGACAATTAGCGGACTGCAGGCCGAGGACGAG | |
| | GCCGATTACTACTGCAGCAGCAACACCCGGTCCAGCACACTGGTTTT | |
| | TGGCGGAGGCACCAAGCTGACAGTGCTGGGATCTAGAGGTGGCGGAG | |
| | GATCTGGCGGCGGAGGAAGCGGAGGCGGCGGATCTCTTGAAATGGCT | |
| | GAAGTGCAGCTGGTGCAGTCTGGCGCCGAGATGAAGAAACCTGGCGC | |
| | CTCTCTGAAGCTGAGCTGCAAGGCCAGCGGCTACACCTTCATCGACT | |
| | ACTACGTGTACTGGATGCGGCAGGCCCCTGGACAGGGACTCGAATCT | |
| | ATGGGCTGGATCAACCCCAATAGCGGCGGCACCAATTACGCCCAGAA | |
| | ATTCCAGGGCAGAGTGACCATGACCAGAGACACCAGCATCAGCACCG | |
| | CCTACATGGAACTGAGCCGGCTGAGATCCGACGACACCGCCATGTAC | |
| | TACTGCGCCAGATCTCAGCGCGACGGCTACATGGATTATTGGGGCCA | |
| | GGGAACCCTGGTCACCGTGTCCAGCGAGTCTAAATACGGACCGCCTT | |
| | GTCCTCCTTGTCCCGCTCCTCCTGTTGCCGGACCTTCCGTGTTCCTG | |
| | TTTCCTCCAAAGCCTAAGGACACCCTGATGATCAGCAGGACCCCTGA | |
| | AGTGACCTGCGTGGTGGTGGATGTGTCCCAAGAGGATCCCGAGGTGC | |
| | AGTTCAACTGGTATGTGGACGGCGTGGAAGTGCACAACGCCAAGACC | |
| | AAGCCTAGAGAGGAACAGTTCCAGAGCACCTACAGAGTGGTGTCCGT | |
| | GCTGACAGTGCTGCACCAGGATTGGCTGAACGGCAAAGAGTACAAGT | |
| | GCAAGGTGTCCAACAAGGGCCTGCCTAGCAGCATCGAGAAAACCATC | |
| | TCCAAGGCCAAGGGCCAGCCAAGAGAGCCCCAGGTTTACACACTGCC | |
| | TCCAAGCCAAGAGGAAATGACCAAGAATCAGGTGTCCCTGACATGCC | |
| | TGGTCAAGGGCTTCTACCCCTCCGATATCGCCGTGGAATGGGAGAGC | |
| | AATGGCCAGCCTGAGAACAACTACAAGACCACACCTCCTGTGCTGGA | |
| | CAGCGACGGCAGTTTCTTCCTGTATAGTAGACTCACCGTGGATAAAT | |
| | CAAGATGGCAAGAGGGCAACGTGTTCAGCTGCAGCGTGATGCACGAG | |
| | GCCCTGCACAACCACTACACCCAGAAAAGCCTGAGCCTGTCTCTGGG | |
| | CAAGATGTTCTGGGTGCTCGTGGTCGTTGGCGGAGTGCTGGCCTGTT | |
| | ACAGCCTGCTGGTTACCGTGGCCTTCATCATCTTTTGGGTCAAGCGG | |
| | GGCAGAAAGAAGCTGCTCTACATCTTCAAGCAGCCCTTCATGCGGCC | |
| | CGTGCAGACCACACAAGAGGAAGATGGCTGCTCCTGCAGATTCCCCG | |
| | AGGAAGAAGAAGGCGGCTGCGAGCTGAGAGTGAAGTTCAGCAGATCC | |
| | GCCGACGCTCCAGCCTATCAGCAGGGCCAAAACCAGCTGTACAACGA | |
| | GCTGAACCTGGGGAGAAGAGAAGAGTACGACGTGCTGGATAAGCGGA | |
| | GAGGCAGAGATCCTGAAATGGGCGGCAAGCCCAGACGGAAGAATCCT | |
| | CAAGAGGGCCTGTATAATGAGCTGCAGAAAGACAAGATGGCCGAGGC | |
| | CTACAGCGAGATCGGAATGAAGGGCGAGCGCAGAAGAGGCAAGGGAC | |
| | ACGATGGACTGTACCAGGGCCTGAGCACCGCCACCAAGGATACCTAT | |
| | GACGCACTGCACATGCAGGCCCTGCCACCTAGA | |
| 304 | ATGCCGCTGCTGCTACTGCTGCCCCTGCTGTGGGCAGGGGCTCTAGC | huCD33 signal |
| | T | peptide (nt) |
| 305 | GAATCCAAATACGGTCCTCCATGCCCTCCATGCCCAGCTCCACCCGT | Long spacer codon |
| | TGCTGGACCAAGCGTGTTTCTGTTCCCACCAAAGCCCAAGGATACAC | diverged (nt) |
| | TCATGATTAGCAGAACCCCAGAAGTGACATGTGTCGTCGTGGACGTT | |
| | TCCCAAGAGGACCCTGAGGTCCAGTTTAACTGGTATGTGGATGGAGT | |
| | GGAGGTGCATAATGCTAAGACTAAGCCAAGAGAAGAGCAGTTTCAGT | |
| | CCACCTATAGAGTGGTCTCCGTGCTCACCGTGCTGCATCAGGACTGG | |
| | CTCAATGGCAAGGAATATAAGTGTAAAGTCTCCAACAAAGGACTGCC | |
| | ATCCAGCATCGAAAAGACCATTAGCAAGGCCAAAGGACAGCCTAGAG | |
| | AGCCTCAGGTCTACACTCTGCCTCCCTCCCAAGAAGAGATGACTAAG | |
| | AACCAGGTCTCTCTGACCTGCCTGGTGAAGGGATTCTACCCTTCCGA | |
| | CATTGCTGTGGAGTGGGAGTCCAATGGACAGCCAGAGAATAACTATA | |
| | AGACTACACCACCCGTGCTCGATAGCGACGGCTCATTCTTTCTGTAC | |
| | TCTCGGCTGACAGTGGACAAGAGCAGATGGCAGGAGGGAAACGTCTT | |
| | TAGCTGCTCCGTGATGCATGAGGCTCTGCATAACCACTATACCCAGA | |
| | AGTCTCTGTCCCTGAGCCTCGGCAAA | |
| 306 | MLQMAGQCSQNEYFDSLLHACIPCQLRCSSNTPPLTCQRYCNASVTN | BCMA protein |
| | SVKGTNAILWTCLGLSLIISLAVFVLMFLLRKINSEPLKDEFKNTGS | (Uniprot Q02223) |
| | GLLGMANIDLEKSRTGDEIILPRGLEYTVEECTCEDCIKSKPKVDSD | |
| | HCFPLPAMEEGATILVTTKTNDYCKSLPAALSATEIEKSTSAR | |
| 307 | ATGTTTTGGGTGCTGGTGGTGGTCGGAGGCGTGCTGGCTTGCTATTC | huCD28 |
| | CCTGCTGGTCACAGTGGCCTTCATTATCTTCTGGGTG | transmembrane |
| | | domain codon |
| | | diverged (nt) |
| 308 | AAACGGGGAAGAAAGAAACTCCTGTATATATTCAAACAACCATTTAT | 4-1BB codon |
| | GAGACCAGTACAAACTACTCAAGAAGAGGATGGCTGTAGCTGCCGGT | diverged (nt) |
| | TTCCCGAAGAAGAGGAAGGAGGATGTGAACTG | |
| 309 | CGGGTGAAGTTCTCCAGAAGCGCCGACGCCCCTGCCTACCAGCAGGG | CD3zeta codon |
| | ACAGAATCAGCTGTATAACGAACTGAACCTGGGCAGAAGGGAGGAAT | diverged (nt) |
| | ACGACGTCCTGGACAAGAGACGAGGCCGGGACCCCGAGATGGGCGGA | |
| | AAGCCTCGGCGGAAGAACCCCCAGGAAGGCCTGTACAACGAACTGCA | |
| | GAAGGACAAGATGGCTGAAGCTTACTCCGAGATCGGCATGAAAGGAG | |
| | AGCGGAGGCGGGGCAAGGGCCACGACGGCCTGTATCAGGGCCTGTCC | |
| | ACCGCCACAAAGGATACCTACGATGCCCTCCATATGCAGGCTCTGCC | |
| | TCCAAGA | |
| 310 | CAGTCTGCCCTGACACAGCCTGCCAGCGTTAGTGCTAGTCCCGGACA | BCMA-55 scFv |
| | GTCTATCGCCATCAGCTGTACCGGCACCAGCTCTGACGTTGGCTGGT | (nt) |
| | ATCAGCAGCACCCTGGCAAGGCCCCTAAGCTGATGATCTACGAGGAC | |
| | AGCAAGAGGCCCAGCGGCGTGTCCAATAGATTCAGCGGCAGCAAGAG | |
| | CGGCAACACCGCCAGCCTGACAATTAGCGGACTGCAGGCCGAGGACG | |
| | AGGCCGATTACTACTGCAGCAGCAACACCCGGTCCAGCACACTGGTT | |
| | TTTGGCGGAGGCACCAAGCTGACAGTGCTGGGATCTAGAGGTGGCGG | |
| | AGGATCTGGCGGCGGAGGAAGCGGAGGCGGCGGATCTCTTGAAATGG | |
| | CTGAAGTGCAGCTGGTGCAGTCTGGCGCCGAGATGAAGAAACCTGGC | |
| | GCCTCTCTGAAGCTGAGCTGCAAGGCCAGCGGCTACACCTTCATCGA | |
| | CTACTACGTGTACTGGATGCGGCAGGCCCCTGGACAGGGACTCGAAT | |
| | CTATGGGCTGGATCAACCCCAATAGCGGCGGCACCAATTACGCCCAG | |
| | AAATTCCAGGGCAGAGTGACCATGACCAGAGACACCAGCATCAGCAC | |
| | CGCCTACATGGAACTGAGCCGGCTGAGATCCGACGACACCGCCATGT | |
| | ACTACTGCGCCAGATCTCAGCGCGACGGCTACATGGATTATTGGGGC | |
| | CAGGGAACCCTGGTCACCGTGTCCAGC | |
| 311 | TCTTCTGAGCTGACCCAAGATCCTGCCGTGTCTGTGGCTCTGGGCCA | GPRC5D scFv |
| | GACAGTGCGGATTACCTGTCAGGGCGATAGCCTGAGAAGCTACTACG | codon diverged |
| | CCAGCTGGTATCAGCAGAAGCCTGGACAGGCTCCCGTGCTGGTCATC | (nt) |
| | TACGGCAAGAACAACAGACCCAGCGGCATCCCCGATAGATTCAGCGG | |
| | AAGCAGCTCTGGCAATACCGCCTCCCTGACAATTACTGGCGCCCAGG | |
| | CCGAAGATGAGGCCGACTACTACTGCAACAGCAGAGACAGCTCCGGC | |
| | AATCCTCCTGTGGTGTTTGGAGGCGGAACCAAACTGACCGTGCTGGG | |
| | CAGCAGAGGTGGAGGTGGAAGCGGCGGTGGAGGCTCCGGAGGAGGCG | |
| | GAAGCCTTGAGATGGCACAGGTGCAGCTGGTGGAATCAGGCGGTGGA | |
| | CTTGTTCACCCTGGCGGAAGCCTGAGACTGTCTTGTGCCGCCAGCGG | |
| | CTTCACCTTCCGGTCCCACAGCATGAACTGGGTCCGACAGGCCCCTG | |
| | GCAAAGGCCTTGAATGGGTGTCCAGCATCAGCAGCGACAGCACCTAC | |
| | ACCTACTATGCCGACAGCGTGAAGGGCAGATTCACCATCTCCAGAGA | |
| | CAACGCCAAGAACAGCCTGTACCTGCAGATGAACTCCCTGAGAGCCG | |
| | AGGACACCGCCGTGTACTACTGTGCTAGAAGTGGCGGCCAGTGGAAG | |
| | TACTACGACTATTGGGGACAGGGCACCCTGGTCACAGTTAGCTCT | |
| 312 | ccatatgagatcttatatggggcacccccgccccttgtaaacttccc | BCMA-41BB |
| | tgaccctgacataacaagagttactaacagcccctctctccaagctc | CAR |
| | acttacaggctctctacttagtccagcacgaagtctggagacctctg | |
| | gcggcagcctaccaagaacaactggaccgaccggtggtacctcaccc | |
| | ttaccgagtcggcgacacagtgtgggtccgccgacaccagactaaga | |
| | acctagaacctcgctggaaaggaccttacacagtcctgctgaccacc | |
| | cccaccgccctcaaagtagacggcatcgcagcttggatacacgccgc | |
| | ccacgtgaaggctgccgaccccgggggtggaccatcctctagactgc | |
| | catggtgctgcagacccaggtgttcatcagcctgctgctgtggatct | |
| | ctggcgcctatggacagtctgccctgacacagcctgccagcgttagt | |
| | gctagtcccggacagtctatcgccatcagctgtaccggcaccagctc | |
| | tgacgttggctggtatcagcagcaccctggcaaggcccctaagctga | |
| | tgatctacgaggacagcaagaggcccagcggcgtgtccaatagattc | |
| | agcggcagcaagagcggcaacaccgccagcctgacaattagcggact | |
| | gcaggccgaggacgaggccgattactactgcagcagcaacacccggt | |
| | ccagcacactggtttttggcggaggcaccaagctgacagtgctggga | |
| | tctagaggtggcggaggatctggcggcggaggaagcggaggcggcgg | |
| | atctcttgaaatggctgaagtgcagctggtgcagtctggcgccgaga | |
| | tgaagaaacctggcgcctctctgaagctgagctgcaaggccagcggc | |
| | tacaccttcatcgactactacgtgtactggatgcggcaggcccctgg | |
| | acagggactcgaatctatgggctggatcaaccccaatagcggcggca | |
| | ccaattacgcccagaaattccagggcagagtgaccatgaccagagac | |
| | accagcatcagcaccgcctacatggaactgagccggctgagatccga | |
| | cgacaccgccatgtactactgcgccagatctcagcgcgacggctaca | |
| | tggattattggggccagggaaccctggtcaccgtgtccagcgagtct | |
| | aaatacggaccgccttgtcctccttgtcccgctcctcctgttgccgg | |
| | accttccgtgttcctgtttcctccaaagcctaaggacaccctgatga | |
| | tcagcaggacccctgaagtgacctgcgtggtggtggatgtgtcccaa | |
| | gaggatcccgaggtgcagttcaactggtatgtggacggcgtggaagt | |
| | gcacaacgccaagaccaagcctagagaggaacagttccagagcacct | |
| | acagagtggtgtccgtgctgacagtgctgcaccaggattggctgaac | |
| | ggcaaagagtacaagtgcaaggtgtccaacaagggcctgcctagcag | |
| | catcgagaaaaccatctccaaggccaagggccagccaagagagcccc | |
| | aggtttacacactgcctccaagccaagaggaaatgaccaagaatcag | |
| | gtgtccctgacatgcctggtcaagggcttctacccctccgatatcgc | |
| | cgtggaatgggagagcaatggccagcctgagaacaactacaagacca | |
| | cacctcctgtgctggacagcgacggcagtttcttcctgtatagtaga | |
| | ctcaccgtggataaatcaagatggcaagagggcaacgtgttcagctg | |
| | cagcgtgatgcacgaggccctgcacaaccactacacccagaaaagcc | |
| | tgagcctgtctctgggcaagatgttctgggtgctcgtggtcgttggc | |
| | ggagtgctggcctgttacagcctgctggttaccgtggccttcatcat | |
| | cttttgggtcaagcggggcagaaagaagctgctctacatcttcaagc | |
| | agcccttcatgcggcccgtgcagaccacacaagaggaagatggctgc | |
| | tcctgcagattccccgaggaagaagaaggcggctgcgagctgagagt | |
| | gaagttcagcagatccgccgacgctccagcctatcagcagggccaaa | |
| | accagctgtacaacgagctgaacctggggagaagagaagagtacgac | |
| | gtgctggataagcggagaggcagagatcctgaaatgggcggcaagcc | |
| | cagacggaagaatcctcaagagggcctgtataatgagctgcagaaag | |
| | acaagatggccgaggcctacagcgagatcggaatgaagggcgagcgc | |
| | agaagaggcaagggacacgatggactgtaccagggcctgagcaccgc | |
| | caccaaggatacctatgacgcactgcacatgcaggccctgccaccta | |
| | ga | |
| 313 | atgccgctgctgctactgctgcccctgctgtgggcaggggctctagc | GPRC5D-41BB |
| | ttcttctgagctgacccaagatcctgccgtgtctgtggctctgggcc | CAR |
| | agacagtgcggattacctgtcagggcgatagcctgagaagctactac | |
| | gccagctggtatcagcagaagcctggacaggctcccgtgctggtcat | |
| | ctacggcaagaacaacagacccagcggcatccccgatagattcagcg | |
| | gaagcagctctggcaataccgccagcctgacaattactggcgcccag | |
| | gccgaagatgaggccgactactactgcaacagcagagacagctccgg | |
| | caatcctcctgtggtttttggcggaggcaccaagctgacagtgctgg | |
| | gaagtagaggtggcggaggatctggcggcggaggaagcggaggcggc | |
| | ggatctcttgaaatggctcaggtgcagctggtggaatcaggcggtgg | |
| | acttgttcaccctggcggaagcctgagactgtcttgtgccgccagcg | |
| | gcttcaccttcagatcccacagcatgaactgggtccgacaggcccct | |
| | ggcaaaggccttgaatgggtgtccagcatcagcagcgacagcaccta | |
| | cacctactatgccgacagcgtgaagggcagattcaccatctccagag | |
| | acaacgccaagaacagcctgtacctgcagatgaactccctgagagcc | |
| | gaggacaccgccgtgtactactgtgctagaagtggcggccagtggaa | |
| | gtactacgactattggggccagggcaccctggtcacagttagctctg | |
| | agtctaaatacggaccgccttgtcctccttgtcccgctcctcctgtt | |
| | gccggaccttccgtgttcctgtttcctccaaagcctaaggacaccct | |
| | gatgatcagcaggacccctgaagtgacctgcgtggtggtggatgtgt | |
| | cccaagaggatcccgaggtgcagttcaactggtatgtggacggcgtg | |
| | gaagtgcacaacgccaagaccaagcctagagaggaacagttccagag | |
| | cacctacagagtggtgtccgtgctgacagtgctgcaccaggattggc | |
| | tgaacggcaaagagtacaagtgcaaggtgtccaacaagggcctgcct | |
| | agcagcatcgagaaaaccatctccaaggccaagggccagccaagaga | |
| | gccccaggtttacacactgcctccaagccaagaggaaatgaccaaga | |
| | atcaggtgtccctgacatgcctggtcaagggcttctacccctccgat | |
| | atcgccgtggaatgggagagcaatggccagcctgagaacaactacaa | |
| | gaccacacctcctgtgctggacagcgacggcagtttcttcctgtata | |
| | gtagactcaccgtggataaatcaagatggcaagagggcaacgtgttc | |
| | agctgcagcgtgatgcacgaggccctgcacaaccactacacccagaa | |
| | aagcctgagcctgtctctgggcaagatgttctgggtgctcgtggtcg | |
| | ttggcggagtgctggcctgttacagcctgctggttaccgtggccttc | |
| | atcatcttttgggtcaagcggggcagaaagaagctgctctacatctt | |
| | caagcagcccttcatgcggcccgtgcagaccacacaagaggaagatg | |
| | gctgctcctgcagattccccgaggaagaagaaggcggctgcgagctg | |
| | agagtgaagttcagcagatccgccgacgctccagcctatcagcaggg | |
| | ccaaaaccagctgtacaacgagctgaacctggggagaagagaagagt | |
| | acgacgtgctggataagcggagaggcagagatcctgaaatgggcggc | |
| | aagcccagacggaagaatcctcaagagggcctgtataatgagctgca | |
| | gaaagacaagatggccgaggcctacagcgagatcggaatgaagggcg | |
| | agcgcagaagaggcaagggacacgatggactgtaccagggcctgagc | |
| | accgccaccaaggatacctatgacgcactgcacatgcaggccctgcc | |
| | acctaga | |
| 314 | atgccgctgctgctactgctgcccctgctgtgggcaggggctctagc | GPRC5D-CD28 |
| | ttcttctgagctgacccaagatcctgccgtgtctgtggctctgggcc | CAR |
| | agacagtgcggattacctgtcagggcgatagcctgagaagctactac | |
| | gccagctggtatcagcagaagcctggacaggctcccgtgctggtcat | |
| | ctacggcaagaacaacagacccagcggcatccccgatagattcagcg | |
| | gaagcagctctggcaataccgccagcctgacaattactggcgcccag | |
| | gccgaagatgaggccgactactactgcaacagcagagacagctccgg | |
| | caatcctcctgtggtttttggcggaggcaccaagctgacagtgctgg | |
| | gaagtagaggtggcggaggatctggcggcggaggaagcggaggcggc | |
| | ggatctcttgaaatggctcaggtgcagctggtggaatcaggcggtgg | |
| | acttgttcaccctggcggaagcctgagactgtcttgtgccgccagcg | |
| | gcttcaccttcagatcccacagcatgaactgggtccgacaggcccct | |
| | ggcaaaggccttgaatgggtgtccagcatcagcagcgacagcaccta | |
| | cacctactatgccgacagcgtgaagggcagattcaccatctccagag | |
| | acaacgccaagaacagcctgtacctgcagatgaactccctgagagcc | |
| | gaggacaccgccgtgtactactgtgctagaagtggcggccagtggaa | |
| | gtactacgactattggggccagggcaccctggtcacagttagctctg | |
| | agtctaaatacggaccgccttgtcctccttgtcccgctcctcctgtt | |
| | gccggaccttccgtgttcctgtttcctccaaagcctaaggacaccct | |
| | gatgatcagcaggacccctgaagtgacctgcgtggtggtggatgtgt | |
| | cccaagaggatcccgaggtgcagttcaactggtatgtggacggcgtg | |
| | gaagtgcacaacgccaagaccaagcctagagaggaacagttccagag | |
| | cacctacagagtggtgtccgtgctgacagtgctgcaccaggattggc | |
| | tgaacggcaaagagtacaagtgcaaggtgtccaacaagggcctgcct | |
| | agcagcatcgagaaaaccatctccaaggccaagggccagccaagaga | |
| | gccccaggtttacacactgcctccaagccaagaggaaatgaccaaga | |
| | atcaggtgtccctgacatgcctggtcaagggcttctacccctccgat | |
| | atcgccgtggaatgggagagcaatggccagcctgagaacaactacaa | |
| | gaccacacctcctgtgctggacagcgacggcagtttcttcctgtata | |
| | gtagactcaccgtggataaatcaagatggcaagagggcaacgtgttc | |
| | agctgcagcgtgatgcacgaggccctgcacaaccactacacccagaa | |
| | aagcctgagcctgtctctgggcaagatgttctgggtgctcgtggtcg | |
| | ttggcggagtgctggcctgttacagcctgctggttaccgtggccttc | |
| | atcatcttttgggtcaggagtaagaggagcaggctcctgcacagtga | |
| | ctacatgaacatgactccccgccgccccgggcccacccgcaagcatt | |
| | accagccctatgccccaccacgcgacttcgcagcctatcgctccaga | |
| | gtgaagttcagcagatccgccgacgctccagcctatcagcagggcca | |
| | aaaccagctgtacaacgagctgaacctggggagaagagaagagtacg | |
| | acgtgctggataagcggagaggcagagatcctgaaatgggcggcaag | |
| | cccagacggaagaatcctcaagagggcctgtataatgagctgcagaa | |
| | agacaagatggccgaggcctacagcgagatcggaatgaagggcgagc | |
| | gcagaagaggcaagggacacgatggactgtaccagggcctgagcacc | |
| | gccaccaaggatacctatgacgcactgcacatgcaggccctgccacc | |
| | taga | |
| 315 | atggtgctgcagacccaggtgttcatcagcctgctgctgtggatctc | BCMA-41BB- |
| | tggcgcctatggacagtctgccctgacacagcctgccagcgttagtg | GPRC5D-41BB |
| | ctagtcccggacagtctatcgccatcagctgtaccggcaccagctct | dual CAR |
| | gacgttggctggtatcagcagcaccctggcaaggcccctaagctgat | |
| | gatctacgaggacagcaagaggcccagcggcgtgtccaatagattca | |
| | gcggcagcaagagcggcaacaccgccagcctgacaattagcggactg | |
| | caggccgaggacgaggccgattactactgcagcagcaacacccggtc | |
| | cagcacactggtttttggcggaggcaccaagctgacagtgctgggat | |
| | ctagaggtggcggaggatctggcggcggaggaagcggaggcggcgga | |
| | tctcttgaaatggctgaagtgcagctggtgcagtctggcgccgagat | |
| | gaagaaacctggcgcctctctgaagctgagctgcaaggccagcggct | |
| | acaccttcatcgactactacgtgtactggatgcggcaggcccctgga | |
| | cagggactcgaatctatgggctggatcaaccccaatagcggcggcac | |
| | caattacgcccagaaattccagggcagagtgaccatgaccagagaca | |
| | ccagcatcagcaccgcctacatggaactgagccggctgagatccgac | |
| | gacaccgccatgtactactgcgccagatctcagcgcgacggctacat | |
| | ggattattggggccagggaaccctggtcaccgtgtccagcgagtcta | |
| | aatacggaccgccttgtcctccttgtcccgctcctcctgttgccgga | |
| | ccttccgtgttcctgtttcctccaaagcctaaggacaccctgatgat | |
| | cagcaggacccctgaagtgacctgcgtggtggtggatgtgtcccaag | |
| | aggatcccgaggtgcagttcaactggtatgtggacggcgtggaagtg | |
| | cacaacgccaagaccaagcctagagaggaacagttccagagcaccta | |
| | cagagtggtgtccgtgctgacagtgctgcaccaggattggctgaacg | |
| | gcaaagagtacaagtgcaaggtgtccaacaagggcctgcctagcagc | |
| | atcgagaaaaccatctccaaggccaagggccagccaagagagcccca | |
| | ggtttacacactgcctccaagccaagaggaaatgaccaagaatcagg | |
| | tgtccctgacatgcctggtcaagggcttctacccctccgatatcgcc | |
| | gtggaatgggagagcaatggccagcctgagaacaactacaagaccac | |
| | acctcctgtgctggacagcgacggcagtttcttcctgtatagtagac | |
| | tcaccgtggataaatcaagatggcaagagggcaacgtgttcagctgc | |
| | agcgtgatgcacgaggccctgcacaaccactacacccagaaaagcct | |
| | gagcctgtctctgggcaagatgttctgggtgctcgtggtcgttggcg | |
| | gagtgctggcctgttacagcctgctggttaccgtggccttcatcatc | |
| | ttttgggtcaagcggggcagaaagaagctgctctacatcttcaagca | |
| | gcccttcatgcggcccgtgcagaccacacaagaggaagatggctgct | |
| | cctgcagattccccgaggaagaagaaggcggctgcgagctgagagtg | |
| | aagttcagcagatccgccgacgctccagcctatcagcagggccaaaa | |
| | ccagctgtacaacgagctgaacctggggagaagagaagagtacgacg | |
| | tgctggataagcggagaggcagagatcctgaaatgggcggcaagccc | |
| | agacggaagaatcctcaagagggcctgtataatgagctgcagaaaga | |
| | caagatggccgaggcctacagcgagatcggaatgaagggcgagcgca | |
| | gaagaggcaagggacacgatggactgtaccagggcctgagcaccgcc | |
| | accaaggatacctatgacgcactgcacatgcaggccctgccacctag | |
| | actcgagggcggcggagagggaaggggatccctcctgacctgtggcg | |
| | acgtcgaggaaaatcctggccccaggatgccgctgctgctactgctg | |
| | cccctgctgtgggcaggggctctagcttcttctgagctgacccaaga | |
| | tcctgccgtgtctgtggctctgggccagacagtgcggattacctgtc | |
| | agggcgatagcctgagaagctactacgccagctggtaccaacaaaag | |
| | cccggacaggctcctgtgctcgtcatttatggcaagaacaacagacc | |
| | ttccggaatccccgataggttttccggaagctcctctggaaatacag | |
| | cttccctcaccatcactggcgcccaagctgaagatgaagctgactat | |
| | tattgtaactccagagacagctccggcaatcctcctgtcgtgttcgg | |
| | aggcggaacaaaattgaccgtgctcggcagcaggggcggaggcggct | |
| | ctggaggtggcgggtccggcggaggaggcagcctggagatggcccag | |
| | gtccaactcgtcgaatcaggcggtggacttgttcatcccggcggaag | |
| | cctgaggctctcttgtgccgcttccggattcacttttagatcccaca | |
| | gcatgaactgggtccgacaagctcccggcaaaggccttgaatgggtg | |
| | tccagcatcagcagcgacagcacctacacctactatgccgacagcgt | |
| | gaaaggaaggttcacaatctctagggacaacgccaagaacagcctgt | |
| | acctgcagatgaactccctcagggctgaggatacagctgtctattat | |
| | tgtgctagaagtggcggccagtggaagtactacgactactggggaca | |
| | aggcacactcgtgacagttagctctgaaagcaagtatggccccccct | |
| | gcccaccctgccctgccccacccgtggctggccccagcgtctttctc | |
| | ttcccccctaaacccaaagatacactcatgatttccagaacacccga | |
| | ggtcacatgtgtcgtcgtcgacgtcagccaggaagaccctgaagtcc | |
| | aatttaattggtacgtcgatggagtcgaggtccataatgctaaaaca | |
| | aaacccagggaagagcaatttcaatccacatatagggtcgtcagcgt | |
| | cctcaccgtcctccatcaagactggctcaatggaaaggaatataaat | |
| | gtaaagtcagcaataaaggactcccctcctccattgaaaagacaatt | |
| | agcaaagctaaaggacaacccagggaacctcaagtgtataccctgcc | |
| | tcccagccaggaagagatgacaaaaaaccaagtcagcctcacctgtc | |
| | tcgtgaaaggattttatcctagcgacattgctgtcgagtgggaatcc | |
| | aacggacaacccgaaaataattataaaacaaccccccccgtcctcga | |
| | ttccgatggaagcttttttctctacagcaggctgacagtcgacaaga | |
| | gcaggtggcaggaaggaaatgtcttttcctgttccgtcatgcatgaa | |
| | gctctccataatcattatacacaaaagtccctctccctcagcctcgg | |
| | aaaaatgttttgggtcctggtcgtggtgggaggcgtcctcgcttgct | |
| | attccctcctcgtgacagtcgcctttattatcttctgggtgaaaaga | |
| | ggaaggaaaaaactcctgtatatttttaaacaaccttttatgagacc | |
| | tgtccaaacaacccaggaagaggacggatgtagctgtaggtttcctg | |
| | aagaggaggagggaggatgtgaactcagggtcaaattttccaggagc | |
| | gctgatgcccccgcttaccaacaaggacagaatcaactctataatga | |
| | actcaatctcggcaggagggaggaatatgatgtcctcgacaaaagaa | |
| | ggggaagggaccccgagatgggaggaaaacctaggagaaaaaacccc | |
| | caggaaggactctacaacgaactccaaaaggataaaatggctgaagc | |
| | ttattccgaaattggcatgaaaggagaaagaaggaggggaaaaggcc | |
| | atgacggcctctatcaaggactctccacagctacaaaagacacatac | |
| | gatgccctccatatgcaagctctcccccccagg | |
| 316 | atggtgctgcagacccaggtgttcatcagcctgctgctgtggatctc | BCMA-41BB- |
| | tggcgcctatggacagtctgccctgacacagcctgccagcgttagtg | GPRC5D-CD28 |
| | ctagtcccggacagtctatcgccatcagctgtaccggcaccagctct | dual CAR |
| | gacgttggctggtatcagcagcaccctggcaaggcccctaagctgat | |
| | gatctacgaggacagcaagaggcccagcggcgtgtccaatagattca | |
| | gcggcagcaagagcggcaacaccgccagcctgacaattagcggactg | |
| | caggccgaggacgaggccgattactactgcagcagcaacacccggtc | |
| | cagcacactggtttttggcggaggcaccaagctgacagtgctgggat | |
| | ctagaggtggcggaggatctggcggcggaggaagcggaggcggcgga | |
| | tctcttgaaatggctgaagtgcagctggtgcagtctggcgccgagat | |
| | gaagaaacctggcgcctctctgaagctgagctgcaaggccagcggct | |
| | acaccttcatcgactactacgtgtactggatgcggcaggcccctgga | |
| | cagggactcgaatctatgggctggatcaaccccaatagcggcggcac | |
| | caattacgcccagaaattccagggcagagtgaccatgaccagagaca | |
| | ccagcatcagcaccgcctacatggaactgagccggctgagatccgac | |
| | gacaccgccatgtactactgcgccagatctcagcgcgacggctacat | |
| | ggattattggggccagggaaccctggtcaccgtgtccagcgagtcta | |
| | aatacggaccgccttgtcctccttgtcccgctcctcctgttgccgga | |
| | ccttccgtgttcctgtttcctccaaagcctaaggacaccctgatgat | |
| | cagcaggacccctgaagtgacctgcgtggtggtggatgtgtcccaag | |
| | aggatcccgaggtgcagttcaactggtatgtggacggcgtggaagtg | |
| | cacaacgccaagaccaagcctagagaggaacagttccagagcaccta | |
| | cagagtggtgtccgtgctgacagtgctgcaccaggattggctgaacg | |
| | gcaaagagtacaagtgcaaggtgtccaacaagggcctgcctagcagc | |
| | atcgagaaaaccatctccaaggccaagggccagccaagagagcccca | |
| | ggtttacacactgcctccaagccaagaggaaatgaccaagaatcagg | |
| | tgtccctgacatgcctggtcaagggcttctacccctccgatatcgcc | |
| | gtggaatgggagagcaatggccagcctgagaacaactacaagaccac | |
| | acctcctgtgctggacagcgacggcagtttcttcctgtatagtagac | |
| | tcaccgtggataaatcaagatggcaagagggcaacgtgttcagctgc | |
| | agcgtgatgcacgaggccctgcacaaccactacacccagaaaagcct | |
| | gagcctgtctctgggcaagatgttctgggtgctcgtggtcgttggcg | |
| | gagtgctggcctgttacagcctgctggttaccgtggccttcatcatc | |
| | ttttgggtcaagcggggcagaaagaagctgctctacatcttcaagca | |
| | gcccttcatgcggcccgtgcagaccacacaagaggaagatggctgct | |
| | cctgcagattccccgaggaagaagaaggcggctgcgagctgagagtg | |
| | aagttcagcagatccgccgacgctccagcctatcagcagggccaaaa | |
| | ccagctgtacaacgagctgaacctggggagaagagaagagtacgacg | |
| | tgctggataagcggagaggcagagatcctgaaatgggcggcaagccc | |
| | agacggaagaatcctcaagagggcctgtataatgagctgcagaaaga | |
| | caagatggccgaggcctacagcgagatcggaatgaagggcgagcgca | |
| | gaagaggcaagggacacgatggactgtaccagggcctgagcaccgcc | |
| | accaaggatacctatgacgcactgcacatgcaggccctgccacctag | |
| | actcgagggcggcggagagggaaggggatccctcctgacctgtggcg | |
| | acgtcgaggaaaatcctggccccaggatgccgctgctgctactgctg | |
| | cccctgctgtgggcaggggctctagcttcttctgagctgacccaaga | |
| | tcctgccgtgtctgtggctctgggccagacagtgcggattacctgtc | |
| | agggcgatagcctgagaagctactacgccagctggtaccaacaaaag | |
| | cccggacaggctcctgtgctcgtcatttatggcaagaacaacagacc | |
| | ttccggaatccccgataggttttccggaagctcctctggaaatacag | |
| | cttccctcaccatcactggcgcccaagctgaagatgaagctgactat | |
| | tattgtaactccagagacagctccggcaatcctcctgtcgtgttcgg | |
| | aggcggaacaaaattgaccgtgctcggcagcaggggcggaggcggct | |
| | ctggaggtggcgggtccggcggaggaggcagcctggagatggcccag | |
| | gtccaactcgtcgaatcaggcggtggacttgttcatcccggcggaag | |
| | cctgaggctctcttgtgccgcttccggattcacttttagatcccaca | |
| | gcatgaactgggtccgacaagctcccggcaaaggccttgaatgggtg | |
| | tccagcatcagcagcgacagcacctacacctactatgccgacagcgt | |
| | gaaaggaaggttcacaatctctagggacaacgccaagaacagcctgt | |
| | acctgcagatgaactccctcagggctgaggatacagctgtctattat | |
| | tgtgctagaagtggcggccagtggaagtactacgactactggggaca | |
| | aggcacactcgtgacagttagctctgaaagcaagtatggccccccct | |
| | gcccaccctgccctgccccacccgtggctggccccagcgtctttctc | |
| | ttcccccctaaacccaaagatacactcatgatttccagaacacccga | |
| | ggtcacatgtgtcgtcgtcgacgtcagccaggaagaccctgaagtcc | |
| | aatttaattggtacgtcgatggagtcgaggtccataatgctaaaaca | |
| | aaacccagggaagagcaatttcaatccacatatagggtcgtcagcgt | |
| | cctcaccgtcctccatcaagactggctcaatggaaaggaatataaat | |
| | gtaaagtcagcaataaaggactcccctcctccattgaaaagacaatt | |
| | agcaaagctaaaggacaacccagggaacctcaagtgtataccctgcc | |
| | tcccagccaggaagagatgacaaaaaaccaagtcagcctcacctgtc | |
| | tcgtgaaaggattttatcctagcgacattgctgtcgagtgggaatcc | |
| | aacggacaacccgaaaataattataaaacaaccccccccgtcctcga | |
| | ttccgatggaagcttttttctctacagcaggctgacagtcgacaaga | |
| | gcaggtggcaggaaggaaatgtcttttcctgttccgtcatgcatgaa | |
| | gctctccataatcattatacacaaaagtccctctccctcagcctcgg | |
| | aaaaatgttttgggtcctggtcgtggtgggaggcgtcctcgcttgct | |
| | attccctcctcgtgacagtcgcctttattatcttctgggtgaggagt | |
| | aagaggagcaggctcctgcacagtgactacatgaacatgactccccg | |
| | ccgccccgggcccacccgcaagcattaccagccctatgccccaccac | |
| | gcgacttcgcagcctatcgctccagggtcaaattttccaggagcgct | |
| | gatgcccccgcttaccaacaaggacagaatcaactctataatgaact | |
| | caatctcggcaggagggaggaatatgatgtcctcgacaaaagaaggg | |
| | gaagggaccccgagatgggaggaaaacctaggagaaaaaacccccag | |
| | gaaggactctacaacgaactccaaaaggataaaatggctgaagctta | |
| | ttccgaaattggcatgaaaggagaaagaaggaggggaaaaggccatg | |
| | acggcctctatcaaggactctccacagctacaaaagacacatacgat | |
| | gccctccatatgcaagctctcccccccagg | |
| 317 | atggtgctgcagacccaggtgttcatcagcctgctgctgtggatctc | GPRC5D-BCMA- |
| | tggcgcctatggacaggtccaactcgtcgaatcaggcggtggacttg | 41BB CAR |
| | ttcatcccggcggaagcctgaggctctcttgtgccgcttccggattt | |
| | acatttagatcccacagcatgaactgggtccgacaagctcccggcaa | |
| | aggccttgaatgggtgtccagcatcagcagcgacagcacctacacct | |
| | actatgccgacagcgtgaaaggaaggttcacaatctctagggacaac | |
| | gccaagaacagcctgtacctgcagatgaactccctcagggctgagga | |
| | tacagctgtctattattgtgctagaagtggcggccagtggaagtact | |
| | acgactactggggacaaggcacactcgtgacagttagctctggcagc | |
| | aggggcggaggcggcagcggaggaggcggctccggcggaggaggcag | |
| | cctggagatggcctcttctgagctgacccaagatcctgccgtgtctg | |
| | tggctctgggccagacagtgcggattacctgtcagggcgatagcctg | |
| | agaagctactacgccagctggtaccaacaaaagcccggacaggctcc | |
| | tgtgctcgtcatttatggcaagaacaacagaccttccggaatccccg | |
| | ataggttttccggaagctcctctggaaatacagcttccctcaccatc | |
| | actggcgcccaagctgaagatgaagctgactattattgtaactccag | |
| | agacagctccggcaatcctcctgtcgtgttcggaggcggaacaaaac | |
| | tcaccgtcctcggaggtggaggatctggaggcggtggaagcggaggg | |
| | ggcggttccggtggcgggggatctcagtctgccctgacacagcctgc | |
| | cagcgttagtgctagtcccggacagtctatcgccatcagctgtaccg | |
| | gcaccagctctgacgttggctggtatcagcagcaccctggcaaggcc | |
| | cctaagctgatgatctacgaggacagcaagaggcccagcggcgtgtc | |
| | caatagattcagcggcagcaagagcggcaacaccgccagcctgacaa | |
| | ttagcggactgcaggccgaggacgaggccgattactactgcagcagc | |
| | aacacccggtccagcacactggtttttggcggaggcaccaagctgac | |
| | agtgctgggatctagaggtggcggaggatctggcggcggaggaagcg | |
| | gaggcggcggatctcttgaaatggctgaagtgcagctggtgcagtct | |
| | ggcgccgagatgaagaaacctggcgcctctctgaagctgagctgcaa | |
| | ggccagcggctacaccttcatcgactactacgtgtactggatgcggc | |
| | aggcccctggacagggactcgaatctatgggctggatcaaccccaat | |
| | agcggcggcaccaattacgcccagaaattccagggcagagtgaccat | |
| | gaccagagacaccagcatcagcaccgcctacatggaactgagccggc | |
| | tgagatccgacgacaccgccatgtactactgcgccagatctcagcgc | |
| | gacggctacatggattattggggccagggaaccctggtcaccgtgtc | |
| | cagcgagtctaaatacggaccgccttgtcctccttgtcccgctcctc | |
| | ctgttgccggaccttccgtgttcctgtttcctccaaagcctaaggac | |
| | accctgatgatcagcaggacccctgaagtgacctgcgtggtggtgga | |
| | tgtgtcccaagaggatcccgaggtgcagttcaactggtatgtggacg | |
| | gcgtggaagtgcacaacgccaagaccaagcctagagaggaacagttc | |
| | cagagcacctacagagtggtgtccgtgctgacagtgctgcaccagga | |
| | ttggctgaacggcaaagagtacaagtgcaaggtgtccaacaagggcc | |
| | tgcctagcagcatcgagaaaaccatctccaaggccaagggccagcca | |
| | agagagccccaggtttacacactgcctccaagccaagaggaaatgac | |
| | caagaatcaggtgtccctgacatgcctggtcaagggcttctacccct | |
| | ccgatatcgccgtggaatgggagagcaatggccagcctgagaacaac | |
| | tacaagaccacacctcctgtgctggacagcgacggcagtttcttcct | |
| | gtatagtagactcaccgtggataaatcaagatggcaagagggcaacg | |
| | tgttcagctgcagcgtgatgcacgaggccctgcacaaccactacacc | |
| | cagaaaagcctgagcctgtctctgggcaagatgttctgggtgctcgt | |
| | ggtcgttggcggagtgctggcctgttacagcctgctggttaccgtgg | |
| | ccttcatcatcttttgggtcaagcggggcagaaagaagctgctctac | |
| | atcttcaagcagcccttcatgcggcccgtgcagaccacacaagagga | |
| | agatggctgctcctgcagattccccgaggaagaagaaggcggctgcg | |
| | agctgagagtgaagttcagcagatccgccgacgctccagcctatcag | |
| | cagggccaaaaccagctgtacaacgagctgaacctggggagaagaga | |
| | agagtacgacgtgctggataagcggagaggcagagatcctgaaatgg | |
| | gcggcaagcccagacggaagaatcotcaagagggcctgtataatgag | |
| | ctgcagaaagacaagatggccgaggcctacagcgagatcggaatgaa | |
| | gggcgagcgcagaagaggcaagggacacgatggactgtaccagggcc | |
| | tgagcaccgccaccaaggatacctatgacgcactgcacatgcaggcc | |
| | ctgccacctaga | |
| 318 | atggtgctgcagacccaggtgttcatcagcctgctgctgtggatctc | BCMA del CAR |
| | tggcgcctatggacagtctgccctgacacagcctgccagcgttagtg | |
| | ctagtcccggacagtctatcgccatcagctgtaccggcaccagctct | |
| | gacgttggctggtatcagcagcaccctggcaaggcccctaagctgat | |
| | gatctacgaggacagcaagaggcccagcggcgtgtccaatagattca | |
| | gcggcagcaagagcggcaacaccgccagcctgacaattagcggactg | |
| | caggccgaggacgaggccgattactactgcagcagcaacacccggtc | |
| | cagcacactggtttttggcggaggcaccaagctgacagtgctgggat | |
| | ctagaggtggcggaggatctggcggcggaggaagcggaggcggcgga | |
| | tctcttgaaatggctgaagtgcagctggtgcagtctggcgccgagat | |
| | gaagaaacctggcgcctctctgaagctgagctgcaaggccagcggct | |
| | acaccttcatcgactactacgtgtactggatgcggcaggcccctgga | |
| | cagggactcgaatctatgggctggatcaaccccaatagcggcggcac | |
| | caattacgcccagaaattccagggcagagtgaccatgaccagagaca | |
| | ccagcatcagcaccgcctacatggaactgagccggctgagatccgac | |
| | gacaccgccatgtactactgcgccagatctcagcgcgacggctacat | |
| | ggattattggggccagggaaccctggtcaccgtgtccagcgagtcta | |
| | aatacggaccgccttgtcctccttgtcccgctcctcctgttgccgga | |
| | ccttccgtgttcctgtttcctccaaagcctaaggacaccctgatgat | |
| | cagcaggacccctgaagtgacctgcgtggtggtggatgtgtcccaag | |
| | aggatcccgaggtgcagttcaactggtatgtggacggcgtggaagtg | |
| | cacaacgccaagaccaagcctagagaggaacagttccagagcaccta | |
| | cagagtggtgtccgtgctgacagtgctgcaccaggattggctgaacg | |
| | gcaaagagtacaagtgcaaggtgtccaacaagggcctgcctagcagc | |
| | atcgagaaaaccatctccaaggccaagggccagccaagagagcccca | |
| | ggtttacacactgcctccaagccaagaggaaatgaccaagaatcagg | |
| | tgtccctgacatgcctggtcaagggcttctacccctccgatatcgcc | |
| | gtggaatgggagagcaatggccagcctgagaacaactacaagaccac | |
| | acctcctgtgctggacagcgacggcagtttcttcctgtatagtagac | |
| | tcaccgtggataaatcaagatggcaagagggcaacgtgttcagctgc | |
| | agcgtgatgcacgaggccctgcacaaccactacacccagaaaagcct | |
| | gagcctgtctctgggcaagatgttctgggtgctcgtggtcgttggcg | |
| | gagtgctggcctgttacagcctgctggttaccgtggccttcatcatc | |
| | ttttgggtc | |
| 319 | ctcgagggcggcggagagggaaggggatccctcctgacctgtggcga | T2A |
| | cgtcgaggaaaatcctggccccagg | |
| 320 | GGGGSGGGGSGGGGSGGGGS | linker |

1. A chimeric antigen receptor comprising:
(1) an extracellular antigen-binding domain that specifically binds human G-protein coupled receptor class C group 5 member D (GPRC5D), wherein the extracellular antigen-binding domain comprises:
(i) a variable heavy chain (V_{H}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:21, 23, 25, 27, 29, 31 or 33; and
(ii) a variable light chain (V_{L}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:22, 24, 26, 28, 30, 32, 34, 63, 64, 65, 66, 67, 68 or 69;
(2) a spacer of at least 125 amino acids in length;
(3) a transmembrane domain; and
(4) an intracellular signaling region.

2. A chimeric antigen receptor comprising:
(1) an extracellular antigen-binding domain that specifically binds human G-protein coupled receptor class C group 5 member D (GPRC5D), wherein the extracellular antigen-binding domain comprises:
(i) a variable heavy chain (V_{H}) region comprising a heavy chain complementarity determining region 1 (CDR-H1), heavy chain complementarity determining region 2 (CDR-H2) and heavy chain complementarity determining region 3 (CDR-H3) contained within the V_{H} region amino acid sequence selected from SEQ ID NOs: 21, 23, 25, 27, 29, 31, and 33; and
(ii) a variable light chain (V_{L}) region comprising a light chain complementarity determining region 1 (CDR-L1), light chain complementarity determining region 2 (CDR-L2) and light chain complementarity determining region 3 (CDR-L3) contained within the V_{L} region amino acid sequence selected from SEQ ID NOs: 22, 24, 26, 28, 30, 32, 34, 63, 64, 65, 66, 67, 68, and 69;
(2) a spacer of at least 125 amino acids in length;
(3) a transmembrane domain; and
(4) an intracellular signaling region.

3. A chimeric antigen receptor comprising:
(1) an extracellular antigen-binding domain that specifically binds human G-protein coupled receptor class C group 5 member D (GPRC5D), wherein the extracellular antigen-binding domain comprises:
(i) a variable heavy chain (V_{H}) region comprising a CDR-H1 comprising the amino acid sequence selected from SEQ ID NOs: 75, 78, 80, 90, 93, 95, 105, 108, 110, 120, 123, 125, 135, 138, 140, 152, 162, 165, and 167; (b) a CDR-H2 comprising the amino acid sequence selected from SEQ ID NOs: 76, 79, 81, 91, 94, 96, 106, 109, 111, 121, 124, 126, 136, 139, 141, 150, 153, 154, 163, 166, and 168; and (c) a CDR-H3 comprising the amino acid sequence selected from SEQ ID NOs: 77, 92, 107, 122, 137, 151, and 164; and
(ii) a variable light chain (V_{L}) region comprising a CDR-L1 comprising the amino acid sequence selected from SEQ ID NOs: 85, 100, 115, 130, 145, 157, and 172; (b) a CDR-L2 comprising the amino acid sequence selected from SEQ ID NOs: 86, 101, 116, 131, 146, and 158; and (c) a CDR-L3 comprising the amino acid sequence selected from SEQ ID NOs: 87, 102, 117, 132, 147, 159, 173, and 297;
(2) a spacer of at least 125 amino acids in length;
(3) a transmembrane domain; and
(4) an intracellular signaling region.

4. The chimeric antigen receptor of claim 2 or claim 3, wherein the extracellular antigen-binding domain comprises:
(i) a variable heavy chain (V_{H}) region comprising: an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:21, 23, 25, 27, 29, 31 or 33; and
(ii) a variable light chain (V_{L}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO:22, 24, 26, 28, 30, 32, 34, 63, 64, 65, 66, 67, 68 or 69.

5. The chimeric antigen receptor of any of claims 1-4, wherein the spacer has a length of between 125 and 300 or of between about 125 and about 300, of between 125 and 250 or of between about 125 and about 250, of between 125 and 230 or of between about 125 and about 230, of between 125 and 200 or of between about 125 and about 200, of between 125 and 180 or of between about 125 and about 180, of between 125 and 150 or of between about 125 and about 150, of between 150 and 300 or of between about 150 and about 300, of between 150 and 250 or of between about 150 and about 250, of between 150 and 230 or of between about 150 and about 230, of between 150 and 200 or of between about 150 and about 200, of between 150 and 180 or of between about 150 and about 180, of between 180 and 300 or of between about 180 and about 300, of between 180 and 250 or of between about 180 and about 250, of between 125 and 300 or of between about 125 and about 300, of between 180 and 230 or of between about 180 and about 230, of between 180 and 200 or of between about 180 and about 200, of between 200 and 300 or of between about 200 and about 300, of between 200 and 250 or of between about 200 and about 250, of between 200 and 230 or of between about 200 and about 230, of between 230 and 300 or of between about 230 and about 300, of between 230 and 250 or of between about 230 and about 250 or of between 250 and 300 or of between about 250 and about 300.

6. The chimeric antigen receptor of any of claims 1-5, wherein:
the spacer is, or is at least about, 130, 140, 150, 160, 170, 180, 190, 200, 210, 220, 221, 222, 223, 224, 225, 226, 227, 228 or 229 amino acids in length, or has a length between any of the foregoing; or
the spacer is about, or is at least about, 130, 140, 150, 160, 170, 180, 190, 200, 210, 220, 221, 222, 223, 224, 225, 226, 227, 228 or 229 amino acids in length, or has a length between any of the foregoing.

7. The chimeric antigen receptor of any of claims 1-6, wherein the spacer comprises a portion of an immunoglobulin.

8. The chimeric antigen receptor of any of claims 1-7, wherein the spacer comprises a sequence of a hinge region, a C_{H}2 and C_{H}3 region.

9. The chimeric antigen receptor of claim 8, wherein:
the hinge region comprises all or a portion of an IgG4 hinge region and/or an IgG2 hinge region, wherein the IgG4 hinge region is optionally a human IgG4 hinge region and the IgG2 hinge region is optionally a human IgG2 hinge region;
the C_{H}2 region comprises all or a portion of an IgG4 C_{H}2 and/or an IgG2 C_{H}2, wherein the IgG4 C_{H}2 is optionally a human IgG4 C_{H}2 and the IgG2 C_{H}2 is optionally a human IgG2 C_{H}2; and/or
the C_{H}3 region comprises all or a portion of an IgG4 C_{H}3 and/or an IgG2 C_{H}3, wherein the IgG4 C_{H}3 is optionally a human IgG4 C_{H}3 and the IgG2 C_{H}3 is optionally a human IgG2 C_{H}3.

10. The chimeric antigen receptor of claim 8 or claim 9, wherein the hinge region, CH2 and CH3 comprises all or a portion of a hinge, all or a portion of a C_{H}2 and all or a portion of a C_{H}3 from human IgG4.

11. The chimeric antigen receptor of claim 8 or claim 9, wherein one or more of the hinge region, the C_{H}2 and the C_{H}3 is chimeric and comprises a hinge, C_{H}2 and C_{H}3 from human IgG4 and human IgG2.

12. The chimeric antigen receptor of any of claims 1-11, wherein the spacer comprises an IgG4/2 chimeric hinge region or a modified IgG4 hinge region comprising at least one amino acid replacement compared to a human IgG4 hinge; an IgG2/4 chimeric C_{H}2 region; and an IgG4 C_{H}3 region.

13. The chimeric antigen receptor of any of claims 1-12, wherein the spacer is or comprises (i) the sequence set forth in SEQ ID NO: 17; (ii) a functional variant of SEQ ID NO:17 that has at least at or about 95%, at or about 96%, at or about 97%, at or about 98% or at or about 99% sequence identity to SEQ ID NO:17; or (iii) a contiguous portion of (i) or (ii) that is at least 125 amino acids in length.

14. The chimeric antigen receptor of any of claims 1-13, wherein the spacer is or comprises the sequence set forth in SEQ ID NO:17.

15. The chimeric antigen receptor of any of claims 1-14, wherein the spacer is or comprises the amino acid sequence encoded by the nucleotide sequence set forth in SEQ ID NO:74.

16. The chimeric antigen receptor of any of claims 1-15, wherein:
the V_{H} region comprises the amino acid sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and the V_{L} region comprises the amino acid sequence set forth in SEQ ID NO:22 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:22;
the V_{H} region comprises the amino acid sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and the V_{L} region comprises the amino acid sequence set forth in SEQ ID NO:63 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:63;
the V_{H} region comprises the amino acid sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and the V_{L} region comprises the amino acid sequence set forth in SEQ ID NO:24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24;
the V_{H} region comprises the amino acid sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and the V_{L} region comprises the amino acid sequence set forth in SEQ ID NO:64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:64;
the V_{H} region comprises the amino acid sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25; and the V_{L} region comprises the amino acid sequence set forth in SEQ ID NO:26 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:26;
the V_{H} region comprises the amino acid sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25; and the V_{L} region comprises the amino acid sequence set forth in SEQ ID NO:65 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:65;
the V_{H} region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the V_{L} region comprises the amino acid sequence set forth in SEQ ID NO:28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:28;
the V_{H} region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the V_{L} region comprises the amino acid sequence set forth in SEQ ID NO:66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:66;
the V_{H} region comprises the amino acid sequence set forth in SEQ ID NO:29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:29; and the V_{L} region comprises the amino acid sequence set forth in SEQ ID NO:30 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:30;
the V_{H} region comprises the amino acid sequence set forth in SEQ ID NO:29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:29; and the V_{L} region comprises the amino acid sequence set forth in SEQ ID NO:67 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:67;
the V_{H} region comprises the amino acid sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and the V_{L} region comprises the amino acid sequence set forth in SEQ ID NO:32 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:32;
the V_{H} region comprises the amino acid sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and the V_{L} region comprises the amino acid sequence set forth in SEQ ID NO:68 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:68;
the V_{H} region comprises the amino acid sequence set forth in SEQ ID NO:33 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:33; and the V_{L} region comprises the amino acid sequence set forth in SEQ ID NO:34 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:34; or
the V_{H} region comprises the amino acid sequence set forth in SEQ ID NO:33 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:33; and the V_{L} region comprises the amino acid sequence set forth in SEQ ID NO:69 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:69.

17. The chimeric antigen receptor of any of claims 1-16, wherein:
the V_{H} region comprises a CDR-H1, CDR-H2, and CDR-H3 comprising the amino acid sequences of SEQ ID NOS:80, 81 and 77, respectively, and the V_{L} region comprises the amino acid sequences of SEQ ID NOS:85, 86 and 87, respectively;
the V_{H} region comprises a CDR-H1, CDR-H2, and CDR-H3 comprising the amino acid sequences of SEQ ID NOS:95, 96, 92, respectively, and the V_{L} region comprises a CDR-L1, CDR-L2, and CDR-L3 comprising the amino acid sequences of SEQ ID NOS:100, 101 and 102, respectively;
the V_{H} region comprises a CDR-H1, CDR-H2, and CDR-H3 comprising the amino acid sequences of SEQ ID NOS:110, 111 and 107, respectively, and the V_{L} region comprises a CDR-L1, CDR-L2, and CDR-L3 comprising the amino acid sequences of SEQ ID NOS:115, 116 and 117, respectively;
the V_{H} region comprises a CDR-H1, CDR-H2, and CDR-H3 comprising the amino acid sequences of SEQ ID NOS:125, 126 and 122, respectively, and the V_{L} region comprises a CDR-L1, CDR-L2, and CDR-L3 comprising the amino acid sequences of SEQ ID NOS:130, 131 and 132, respectively;
the V_{H} region comprises a CDR-H1, CDR-H2, and CDR-H3 comprising the amino acid sequences of SEQ ID NOS:140, 141 and 137, respectively, and the V_{L} region comprises a CDR-L1, CDR-L2, and CDR-L3 comprising the amino acid sequences of SEQ ID NOS:145, 146 and 147, respectively;
the V_{H} region comprises a CDR-H1, CDR-H2, and CDR-H3 comprising the amino acid sequences of SEQ ID NOS:140, 154 and 151, respectively, and the V_{L} region comprises a CDR-L1, CDR-L2, and CDR-L3 comprising the amino acid sequences of SEQ ID NOS:157, 158 and 159, respectively;
the V_{H} region comprises a CDR-H1, CDR-H2, and CDR-H3 comprising the amino acid sequences of SEQ ID NOS:167, 168 and 164, respectively, and the V_{L} region comprises a CDR-L1, CDR-L2, and CDR-L3 comprising the amino acid sequences of SEQ ID NOS:172, 86, 173, respectively; or
the V_{H} region comprises a CDR-H1, CDR-H2, and CDR-H3 comprising the amino acid sequences of SEQ ID NOS:169, 170 and 171, respectively, and the V_{L} region comprises a CDR-L1, CDR-L2, and CDR-L3 comprising the amino acid sequences of SEQ ID NOS:174, 89 and 297, respectively.

18. The chimeric antigen receptor of any of claims 1-17, wherein:
the V_{H} region and the V_{L} region comprise the amino acid sequence set forth in SEQ ID NOs:21 and 22, respectively or the amino acid sequence set forth in SEQ ID NOs: 21 and 63, respectively;
the V_{H} region and the V_{L} region comprise the amino acid sequence set forth in SEQ ID NOs:23 and 24, respectively or the amino acid sequence set forth in SEQ ID NOs:23 and 64, respectively;
the V_{H} region and the V_{L} region comprise the amino acid sequence set forth in SEQ ID NOs:25 and 26, respectively or the amino acid sequence set forth in SEQ ID NOs: 25 and 65, respectively;
the V_{H} region and the V_{L} region comprise the amino acid sequence set forth in SEQ ID NOs:27 and 28, respectively or the amino acid sequence set forth in SEQ ID NOs: 27 and 66, respectively;
the V_{H} region and the V_{L} region comprise the amino acid sequence set forth in SEQ ID NOs:29 and 30, respectively or the amino acid sequence set forth in SEQ ID NOs:29 and 67, respective;
the V_{H} region and the V_{L} region comprise the amino acid sequence set forth in SEQ ID NOs:31 and 32, respectively or the amino acid sequence set forth in SEQ ID Nos: 31 and 68, respectively; or
the V_{H} region and the V_{L} region comprise the amino acid sequence set forth in SEQ ID NOs:33 and 34 or the amino acid sequence set forth in SEQ ID Nos: 33 and 69, respectively, respectively.

19. The chimeric antigen receptor of any of claims 1-18, wherein:
the V_{H} region comprises the amino acid sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and the V_{L} region comprises the amino acid sequence set forth in SEQ ID NO:22 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:22;
the V_{H} region comprises the amino acid sequence set forth in SEQ ID NO:21 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:21; and the V_{L} region comprises the amino acid sequence set forth in SEQ ID NO:63 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:63;
the V_{H} region comprises the amino acid sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and the V_{L} region comprises the amino acid sequence set forth in SEQ ID NO:24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24;
the V_{H} region comprises the amino acid sequence set forth in SEQ ID NO:23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:23; and the V_{L} region comprises the amino acid sequence set forth in SEQ ID NO:64 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:64;
the V_{H} region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the V_{L} region comprises the amino acid sequence set forth in SEQ ID NO:28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:28;
the V_{H} region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the V_{L} region comprises the amino acid sequence set forth in SEQ ID NO:66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:66;
the V_{H} region comprises the amino acid sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and the V_{L} region comprises the amino acid sequence set forth in SEQ ID NO:32 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:32; or
the V_{H} region comprises the amino acid sequence set forth in SEQ ID NO:31 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:31; and the V_{L} region comprises the amino acid sequence set forth in SEQ ID NO:68 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:68.

20. The chimeric antigen receptor of any of claims 1-19, wherein:
the V_{H} region comprises a CDR-H1, CDR-H2, and CDR-H3 comprising the amino acid sequences of SEQ ID NOS:80, 81 and 77, respectively, and the V_{L} region comprises a CDR-L1, CDR-L2, and CDR-L3 comprising the amino acid sequences of SEQ ID NOS:85, 86 and 87, respectively;
the V_{H} region comprises a CDR-H1, CDR-H2, and CDR-H3 comprising the amino acid sequences of SEQ ID NOS:95, 96, 92, respectively, and the V_{L} region comprises a CDR-L1, CDR-L2, and CDR-L3 comprising the amino acid sequences of SEQ ID NOS:100, 101 and 102, respectively;
the V_{H} region comprises a CDR-H1, CDR-H2, and CDR-H3 comprising the amino acid sequences of SEQ ID NOS:125, 126 and 122, respectively, and the V_{L} region comprises a CDR-L1, CDR-L2, and CDR-L3 comprising the amino acid sequences of SEQ ID NOS:130, 131 and 132, respectively; or
the V_{H} region comprises a CDR-H1, CDR-H2, and CDR-H3 comprising the amino acid sequences of SEQ ID NOS:140, 154 and 151, respectively, and the V_{L} region comprises a CDR-L1, CDR-L2, and CDR-L3 comprising the amino acid sequences of SEQ ID NOS:157, 158 and 159, respectively.

21. The chimeric antigen receptor of any of claims 1-20, wherein:
the V_{H} region and the V_{L} region comprise the amino acid sequence set forth in SEQ ID NOs:21 and 22, respectively or the amino acid sequence set forth in SEQ ID Nos: 21 and 63, respectively;
the V_{H} region and the V_{L} region comprise the amino acid sequence set forth in SEQ ID NOs:23 and 24, respectively or the amino acid sequence set forth in SEQ ID Nos: 23 and 64, respectively;
the V_{H} region and the V_{L} region comprise the amino acid sequence set forth in SEQ ID NOs:27 and 28, respectively or the amino acid sequence set forth in SEQ ID Nos: 27 and 66, respectively; or
the V_{H} region and the V_{L} region comprise the amino acid sequence set forth in SEQ ID NOs:31 and 32, respectively or the amino acid sequence set forth in SEQ ID Nos: 31 and 68, respectively.

22. The chimeric antigen receptor of any of claims 1-21, wherein:
the V_{H} region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the V_{L} region comprises the amino acid sequence set forth in SEQ ID NO:28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:28; or
the V_{H} region comprises the amino acid sequence set forth in SEQ ID NO:27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:27; and the V_{L} region comprises the amino acid sequence set forth in SEQ ID NO:66 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:66.

23. The chimeric antigen receptor of any of claims 1-22, comprising a variable heavy chain (V_{H}) region comprising a CDR-H1, CDR-H2 and CDR-H3 contained within the V_{H} region amino acid sequence set forth in SEQ ID NO: 27; and a variable light chain (V_{L}) region comprising a CDR-L1, CDR-L2 and CDR-L3 contained within the V_{L} region amino acid sequence set forth in SEQ ID NO: 28 or 66.

24. The chimeric antigen receptor of any of claims 1-23, wherein the V_{H} region comprises the amino acid sequence of SEQ ID NOS:125, 126 and 122, respectively, and the V_{L} region comprises the amino acid sequence of SEQ ID NOS:130, 131 and 132, respectively.

25. The chimeric antigen receptor of any of claims 1-23, wherein the V_{H} region comprises the amino acid sequence of SEQ ID NOS:127, 128 and 129, respectively, and the V_{L} region comprises the amino acid sequence of SEQ ID NOS:133, 134 and 132, respectively.

26. The chimeric antigen receptor of any of claims 1-23, wherein the V_{H} region comprises the amino acid sequence of SEQ ID NOS:120, 121 and 122, respectively, and the V_{L} region comprises the amino acid sequence of SEQ ID NOS:130, 131 and 132, respectively.

27. The chimeric antigen receptor of any of claims 1-23, wherein the V_{H} region comprises the amino acid sequence of SEQ ID NOS:123, 124 and 122, respectively, and the V_{L} region comprises the amino acid sequence of SEQ ID NOS:130, 131 and 132, respectively.

28. The chimeric antigen receptor of any of claims 1-23, wherein the V_{H} region and the V_{L} regions comprise the amino acid sequence set forth in SEQ ID NOs:27 and 28, respectively or the amino acid sequence set forth in SEQ ID Nos: 27 and 66, respectively.

29. The chimeric antigen receptor of any of claims 1-28, wherein the extracellular antigen-binding domain is a single chain antibody fragment.

30. The chimeric antigen receptor of any of claims 1-29, wherein the single chain antibody fragment is or comprises a single chain variable fragment (scFv).

31. The chimeric antigen receptor of any of claims 1-30, when the V_{H} region and the V_{L} region are joined by a flexible linker.

32. The chimeric antigen receptor of claim 31, wherein the linker comprises the amino acid sequence GGGGSGGGGSGGGGS (SEQ ID NO:52).

33. The chimeric antigen receptor of any of claims 1-32, wherein the V_{H} region is amino-terminal to the V_{L} region.

34. The chimeric antigen receptor of any of claims 1-33, wherein:
the extracellular antigen-binding domain comprises an amino acid sequence selected from SEQ ID NOs: 1, 3, 5, 7, 9, 11, and 13 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 1, 3, 5, 7, 9, 11, and 13; and/or
the extracellular antigen-binding domain is encoded by the nucleotide sequence selected from SEQ ID Nos: 257, 259, 261, 263, 265, 267, and 269 or a nucleotide sequence having at least at or about 90%, at or about 91%, at or about 92%, at or about 93%, at or about 94%, at or about 95%, at or about 96%, at or about 97%, at or about 98%, or at or about 99% sequence identity to the nucleotide sequence selected from SEQ ID Nos: 257, 259, 261, 263, 265, 267, and 269.

35. The chimeric antigen receptor of any of claims 1-34, wherein the extracellular antigen-binding domain comprises the amino acid sequence selected from SEQ ID NOs: 1, 3, 5, 7, 9, 11, and 13.

36. The chimeric antigen receptor of any of claims 1-34, wherein the extracellular antigen-binding domain comprises the amino acid sequence selected from SEQ ID NOs: 1, 3, 7, and 11 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 1, 3, 7, and 11.

37. The chimeric antigen receptor of any of claims 1-36, wherein the antigen-binding domain comprises the amino acid sequence selected from SEQ ID NOs: 1, 3, 7, and 11.

38. The chimeric antigen receptor of any of claim 1-34 or 37, wherein the antigen-binding domain comprises the amino acid sequence set forth in SEQ ID NO: 7 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 7.

39. The chimeric antigen receptor of any of claims 1-38, wherein the antigen-binding domain comprises the amino acid sequence set forth in SEQ ID NO: 7.

40. The chimeric antigen receptor of any of claims 1-32, wherein the V_{H} region is carboxy-terminal to the V_{L} region.

41. The chimeric antigen receptor of any of claims 1-32 and 40, wherein:
the extracellular antigen-binding domain comprises the amino acid sequence selected from SEQ ID NOs: 2, 4, 6, 8, 10, 12, and 14 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 2, 4, 6, 8, 10, 12, and 14; and/or
the extracellular antigen-binding domain is encoded by the nucleotide sequence selected from SEQ ID Nos: 258, 260, 262, 264, 266, 268, and 270 or a nucleotide sequence having at least at or about 90%, at or about 91%, at or about 92%, at or about 93%, at or about 94%, at or about 95%, at or about 96%, at or about 97%, at or about 98%, or at or about 99% sequence identity to the nucleotide sequence selected from SEQ ID Nos: 258, 260, 262, 264, 266, 268, and 270.

42. The chimeric antigen receptor of any of claims 1-32, 40 and 41, wherein the extracellular antigen-binding domain comprises the amino acid sequence selected from SEQ ID NOs: 2, 4, 6, 8, 10, 12, and 14.

43. The chimeric antigen receptor of any of claims 1-32, 40 and 41, wherein the extracellular antigen-binding domain comprises the amino acid sequence selected from SEQ ID NOs: 2, 4, 8, and 12 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 2, 4, 8, and 12.

44. The chimeric antigen receptor of any of claim 1-32 or 40-43, wherein the extracellular antigen-binding domain comprises the amino acid sequence selected from SEQ ID NOs: 2, 4, 8, and 12.

45. The chimeric antigen receptor of any of claims 1-32, 40, 41 and 43, wherein the antigen-binding domain comprises the amino acid sequence set forth in SEQ ID NO: 8 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 8.

46. The chimeric antigen receptor of any of claim 1-32 or 40-45, wherein the extracellular antigen-binding domain comprises the amino acid sequence set forth in SEQ ID NO: 8.

47. The chimeric antigen receptor of any of claims 1-46, wherein the intracellular signaling region comprises an intracellular cytoplasmic signaling domain.

48. The chimeric antigen receptor of claim 47, wherein the intracellular signaling domain is or comprises a cytoplasmic signaling domain of a CD3-zeta (CD3ζ) chain or a functional variant or signaling portion thereof.

49. The chimeric antigen receptor of any of claim 47 or 48, wherein the intracellular signaling domain is or comprises the amino acid sequence set forth in SEQ ID NO:20 or an amino acid that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.

50. The chimeric antigen receptor of any of claims 47-49, wherein the intracellular signaling region further comprises a costimulatory signaling region.

51. The chimeric antigen receptor of claim 50, wherein the costimulatory signaling region comprises an intracellular signaling domain of CD28, 4-1BB, or ICOS, or a signaling portion thereof.

52. The chimeric antigen receptor of any of claim 50 or 51, wherein the costimulatory signaling region comprises an intracellular signaling domain of CD28.

53. The chimeric antigen receptor of any of claims 50-52, wherein the costimulatory signaling region is or comprises the amino acid sequence set forth in SEQ ID NO:46 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.

54. The chimeric antigen receptor of any of claim 50 or 51, wherein the costimulatory signaling region comprises an intracellular signaling domain of 4-1BB.

55. The chimeric antigen receptor of any of claims 50, 51, and 54, wherein the costimulatory signaling region is or comprises the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.

56. The chimeric antigen receptor of any of claims 50-55, wherein the costimulatory signaling region is between the transmembrane domain and the intracellular signaling region.

57. The chimeric antigen receptor of any of claims 1-56, wherein the transmembrane domain is or comprises a transmembrane domain from CD4, CD28, or CD8.

58. The chimeric antigen receptor of any of claims 1-57, wherein the transmembrane domain is or comprises a transmembrane domain derived from CD28.

59. The chimeric antigen receptor of any of claims 1-58, wherein the transmembrane domain is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:18.

60. A chimeric antigen receptor comprising:
(1) an extracellular antigen-binding domain that specifically binds human G-protein coupled receptor class C group 5 member D (GPRC5D), wherein the extracellular antigen-binding domain comprises:
(i) a variable heavy chain (V_{H}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 27; and
(ii) a variable light chain (V_{L}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 28 or 66;
(2) a spacer comprising an IgG4/2 chimeric hinge or a modified IgG4 hinge; an IgG2/4 chimeric C_{H}2 region; and an IgG4 C_{H}3 region, optionally that is about 228 amino acids in length or a spacer set forth in SEQ ID NO:17;
(3) a transmembrane domain from human CD28; and
(4) an intracellular signaling region comprising a cytoplasmic signaling domain of a CD3-zeta (CD3ζ) chain and an intracellular signaling domain of a T cell costimulatory molecule.

61. The chimeric antigen receptor of claim 60, wherein:
the V_{H} region comprises a CDR-H1, CDR-H2 and CDR-H3 contained within the V_{H} region amino acid sequence set forth in SEQ ID NO: 27; and the V_{L} region comprises a CDR-L1, CDR-L2 and CDR-L3 contained within the V_{L} region amino acid sequence set forth in SEQ ID NO: 28 or 66; or
the V_{H} region a CDR-H1, CDR-H2, and CDR-H3 comprising the amino acid sequence of SEQ ID NOS:125, 126 and 122, respectively, and the V_{L} region comprises a CDR-L1, CDR-L2, and CDR-L3 comprising the amino acid sequence of SEQ ID NOS:130, 131 and 132, respectively;
the V_{H} region comprises a CDR-H1, CDR-H2, and CDR-H3 comprising the amino acid sequence of SEQ ID NOS:120, 121 and 122, respectively, and the V_{L} region comprises a CDR-L1, CDR-L2, and CDR-L3 comprising the amino acid sequence of SEQ ID NOS:130, 131 and 132, respectively; or
the V_{H} region comprises a CDR-H1, CDR-H2, and CDR-H3 comprising the amino acid sequence of SEQ ID NOS:123, 124 and 122, respectively, and the V_{L} region comprises a CDR-L1, CDR-L2, and CDR-L3 comprising the amino acid sequence of SEQ ID NOS:130, 131 and 132, respectively.

62. A chimeric antigen receptor comprising:
(1) an extracellular antigen-binding domain that specifically binds human G-protein coupled receptor class C group 5 member D (GPRC5D), wherein the extracellular antigen-binding domain comprises:
a V_{H} region comprising a CDR-H1, CDR-H2 and CDR-H3 contained within the V_{H} region amino acid sequence set forth in SEQ ID NO: 27; and a variable light (V_{L}) region comprising a CDR-L1, CDR-L2 and CDR-L3 contained within the V_{L} region amino acid sequence set forth in SEQ ID NO: 28 or 66; or
a V_{H} region comprising a CDR-H1, CDR-H2, and CDR-H3 comprising the amino acid sequence of SEQ ID NOS:125, 126 and 122, respectively, and a V_{L} region comprising a CDR-L1, CDR-L2, and CDR-L3 comprising the amino acid sequence of SEQ ID NOS:130, 131 and 132, respectively;
a V_{H} region comprising a CDR-H1, CDR-H2, and CDR-H3 comprising the amino acid sequence of SEQ ID NOS:120, 121 and 122, respectively, and a V_{L} region comprising a CDR-L1, CDR-L2, and CDR-L3 comprising the amino acid sequence of SEQ ID NOS:130, 131 and 132, respectively; or
a V_{H} region comprising a CDR-H1, CDR-H2, and CDR-H3 comprising the amino acid sequence of SEQ ID NOS:123, 124 and 122, respectively, and a V_{L} region comprising a CDR-L1, CDR-L2, and CDR-L3 comprising the amino acid sequence of SEQ ID NOS:130, 131 and 132, respectively;
(2) a spacer comprising an IgG4/2 chimeric hinge or a modified IgG4 hinge; an IgG2/4 chimeric C_{H}2 region; and an IgG4 C_{H}3 region, optionally that is about 228 amino acids in length or a spacer set forth in SEQ ID NO:17;
(3) a transmembrane domain from human CD28; and
(4) an intracellular signaling region comprising a cytoplasmic signaling domain of a human CD3-zeta (CD3ζ) chain and an intracellular signaling domain of a human CD28 or a human 4-1BB.

63. The chimeric antigen receptor of any of claims 60-62, wherein:
the extracellular antigen-binding domain comprises the V_{H} region amino acid sequence set forth in SEQ ID NO:27 and the V_{L} region amino acid sequence set forth in SEQ ID NO:28 or 66; and/or
the extracellular antigen-binding domain comprises an scFv set forth in SEQ ID NO:7 or SEQ ID NO:8.

64. The chimeric antigen receptor of any of claims 60-63, wherein the transmembrane domain is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.

65. The chimeric antigen receptor of any of claims 60-64, wherein the intracellular signaling region comprises (a) the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20 and (b) the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.

66. The chimeric antigen receptor of any of claims 60-65, wherein the intracellular signaling region is or comprises the sequences set forth in SEQ ID NO:20 and SEQ ID NO:46.

67. The chimeric antigen receptor of any of claims 60-64, wherein the intracellular signaling region comprises (a) the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20 and (b) the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.

68. A polynucleotide comprising a nucleotide sequence encoding the chimeric antigen receptor of any of claims 1-67.

69. The polynucleotide of claim 68, wherein the nucleic acid encoding the spacer comprises at least one modified splice donor and/or splice acceptor site, said modified splice donor and/or acceptor site comprising one or more nucleotide modifications corresponding to a reference splice donor site and/or reference splice acceptor site contained in the sequence set forth in SEQ ID NO:73.

70. The polynucleotide of claim 69, wherein the one or more nucleotide modifications comprise an amino acid substitution.

71. The polynucleotide of any of claims 69 and 70, wherein:
the reference splice donor and/or reference splice acceptor site(s) has a splice site prediction score of at least at or about 0.4, at or about 0.5, at or about 0.6, at or about 0.70, at or about 0.75, at or about 0.80, at or about 0.85, at or about 0.90, at or about 0.95, at or about 0.99, or at or about 1.0; and/or
the reference splice donor and/or reference splice acceptor site(s) is/are predicted to be involved in a splice event with a probability of at least at or about 40%, at or about 50%, at or about 60%, at or about 70%, at or about 75%, at or about 80%, at or about 85%, at or about 90%, at or about 95%, at or about 99%, or at or about 100%.

72. The polynucleotide of any of claims 69-71, wherein:
the reference splice donor site comprises the sequence aatctaagtacggac (SEQ ID NO: 176), tcaactggtacgtgg (SEQ ID NO:177), acaattagtaaggca (SEQ ID NO:178) and/or accacaggtgtatac (SEQ ID NO:179); and/or
the reference splice acceptor site comprises the sequence aagtttctttctgtattccaggctgaccgtggataaatctc (SEQ ID NO:180) and/or gggcaacgtgttctcttgcagtgtcatgcacgaagccctgc (SEQ ID NO:181).

73. The polynucleotide of any of claims 69-72, wherein:
the reference splice donor site comprises the sequence tcaactggtacgtgg (SEQ ID NO:177); and/or
the reference splice acceptor site comprises the sequence aagtttctttctgtattccaggctgaccgtggataaatctc (SEQ ID NO:180).

74. The polynucleotide of any of claims 69-73, wherein the one or more nucleotide modifications is silent and/or results in a degenerate codon compared to SEQ ID NO:73 and/or does not change the amino acid sequence of the encoded spacer.

75. The polynucleotide of any of claims 69-74, wherein:
the modified splice donor site is set forth in agtctaaatacggac (SEQ ID NO:182), tcaactggtatgtgg (SEQ ID NO:183), accatctccaaggcc (SEQ ID NO:184) and/or gccccaggtttacac (SEQ ID NO:185); and/or
the modified splice acceptor site is set forth in cagtttcttcctgtatagtagactcaccgtggataaatcaa (SEQ ID NO:186), gggcaacgtgttcagctgcagcgtgatgcacgaggccctgc (SEQ ID NO: 187) and/or cgccttgtcctccttgtcccgctcctcctgttgccggacct (SEQ ID NO:188).

76. The polynucleotide of any of claims 69-75, wherein the modified splice donor site is set forth in tcaactggtatgtgg (SEQ ID NO:183) and/or the modified acceptor site is set forth in cagtttcttcctgtatagtagactcaccgtggataaatcaa (SEQ ID NO:186) and/or cgccttgtcctccttgtcccgctcctcctgttgccggacct (SEQ ID NO:188).

77. The polynucleotide of any of claims 69-76, wherein the spacer is encoded by the nucleotide sequence set forth in SEQ ID NO:74.

78. The polynucleotide of any of claims 68-77, wherein, upon expression in a cell, the transcribed RNA, optionally messenger RNA (mRNA), from the polynucleotide exhibits reduced heterogeneity compared to the heterogeneity of the mRNA transcribed from a reference polynucleotide, said reference polynucleotide encoding the same amino acid sequence as the polynucleotide, wherein the reference polynucleotide differs by the presence of one or more splice donor site and/or one or more splice acceptor site in the nucleic acid encoding the spacer and/or comprises one or more nucleotide modifications compared to the polynucleotide and/or comprises the spacer set forth in SEQ ID NO:73.

79. The polynucleotide of any of claims 68-78, wherein the polynucleotide is codon-optimized for expression in a human cell.

80. The polynucleotide of any of claims 68-78, wherein the chimeric antigen receptor is a first chimeric antigen receptor and the polynucleotide further comprises a nucleotide sequence encoding a second chimeric antigen receptor.

81. The polynucleotide of claim 80, wherein the first and second chimeric antigen receptors are separated by one or more multicistronic element(s).

82. The polynucleotide of claim 81, wherein the one or more multicistronic element is or comprises a ribosome skip sequence, optionally wherein the ribosome skip sequence is a T2A, a P2A, an E2A, or an F2A element.

83. The polynucleotide of claim 82, wherein the nucleotide sequence encoding the one or more multicistronic element is codon diverged.

84. The polynucleotide of claim 82 or claim 83, wherein the nucleotide sequence encoding the one or more multicistronic element is or comprises the sequence set forth in SEQ ID NO:319.

85. The polynucleotide of any of claims 80-84, wherein the second chimeric antigen receptor (CAR) comprises an extracellular antigen-binding domain that specifically binds a second antigen expressed on or associated with multiple myeloma.

86. The polynucleotide of 85, wherein the second CAR further comprises a spacer, a transmembrane domain, and an intracellular signaling region.

87. The polynucleotide of claim 85 or claim 86, wherein the second antigen is selected from B cell maturation antigen (BCMA), CD38, CD138, CS-1, BAFF-R, TACI, and FcRH5.

88. The polynucleotide of any of claims 85-87, wherein the second antigen is BCMA.

89. The polynucleotide of any of claims 85-88, wherein the second CAR comprises:
(1) an extracellular antigen-binding domain that specifically binds BCMA, wherein the extracellular antigen-binding domain comprises:
(i) a variable heavy chain (V_{H}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 189, 191, 193, 195 or 197; and
(ii) a variable light chain (V_{L}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 190, 192, 194, 196 or 198;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length, or a spacer set forth in SEQ ID NO:17;
(3) a transmembrane domain; and
(4) an intracellular signaling region.

90. The polynucleotide of claim 89, wherein the V_{H} region of the second CAR comprises a CDR-H1, CDR-H2 and CDR-H3 contained within the V_{H} region amino acid sequence set forth in SEQ ID NO: 189, 191, 193, 195 or 197; and the V_{L} region of the second CAR comprises a CDR-L1, CDR-L2 and CDR-L3 contained within the V_{L} region amino acid sequence set forth in SEQ ID NO: 190, 192, 194, 196 or 198.

91. The polynucleotide of any of claims 85-88, wherein the second CAR comprises:
(1) an extracellular antigen-binding domain that specifically binds BCMA, wherein the extracellular antigen-binding domain comprises:
(i) a variable heavy chain (V_{H}) region comprising a heavy chain complementarity determining region 1 (CDR-H1) comprising the amino acid sequence selected from SEQ ID NOs: 199, 202, 206, and 209; (b) a heavy chain complementarity determining region 2 (CDR-H2) comprising the amino acid sequence selected from SEQ ID NOs: 200, 203, 207, and 210; and (c) a heavy chain complementarity determining region 3 (CDR-H3) comprising the amino acid sequence selected from SEQ ID NOs: 201, 204, 205, 208, and 211; and
(ii) a variable light chain (V_{L}) region comprising a light chain complementarity determining region 1 (CDR-L1) comprising the amino acid sequence selected from SEQ ID NOs: 218, 221, 224, 227, and 230; (b) a light chain complementarity determining region 2 (CDR-L2) comprising the amino acid sequence selected from any one of SEQ ID NOs: 219, 222, 225, 228, and 231; and (c) a light chain complementarity determining region 3 (CDR-L3) comprising the amino acid sequence selected from SEQ ID NOs: 220, 223, and 226;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is at or about 228 amino acids in length, or a spacer set forth in SEQ ID NO:17;
(3) a transmembrane domain; and
(4) an intracellular signaling region.

92. The polynucleotide of any of claims 89-91, wherein:
the V_{H} region of the second CAR comprises a CDR-H1, CDR-H2, and CDR-H3 comprising the amino acid sequence of SEQ ID NOS:199, 200 and 201, respectively, and the V_{L} region of the second CAR comprises a CDR-L1, CDR-L2, and CDR-L3 comprising the amino acid sequence of SEQ ID NOS:218, 219 and 220, respectively;
the V_{H} region of the second CAR comprises a CDR-H1, CDR-H2, and CDR-H3 comprising the amino acid sequence of SEQ ID NOS:202, 203, 204, respectively, and the V_{L} region of the second CAR comprises a CDR-L1, CDR-L2, and CDR-L3 comprising the amino acid sequence of SEQ ID NOS:221, 222 and 223, respectively;
the V_{H} region of the second CAR comprises a CDR-H1, CDR-H2, and CDR-H3 comprising the amino acid sequence of SEQ ID NOS:199, 200, 205, respectively, and the V_{L} region of the second CAR comprises a CDR-L1, CDR-L2, and CDR-L3 comprising the amino acid sequence of SEQ ID NOS:224, 225, and 226, respectively;
the V_{H} region of the second CAR comprises a CDR-H1, CDR-H2, and CDR-H3 comprising the amino acid sequence of SEQ ID NOS:206, 207, 208, respectively, and the V_{L} region of the second CAR comprises a CDR-L1, CDR-L2, and CDR-L3 comprising the amino acid sequence of SEQ ID NOS:227, 228 and 229, respectively; or
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively.

93. The polynucleotide of any of claims 89-91, wherein:
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively.

94. The polynucleotide of any of claims 89-93, wherein:
the V_{H} region and V_{L} region of the second CAR comprise (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the V_{H} region and V_{L} region of the second CAR comprise (a) the amino acid sequences set forth in SEQ ID NO:191 and SEQ ID NO:192, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the V_{H} region and V_{L} region of the second CAR comprise (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the V_{H} region and V_{L} region of the second CAR comprise (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the V_{H} region and V_{L} region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:198.

95. The polynucleotide of any of claims 89-94, wherein:
the V_{H} region and V_{L} region of the second CAR comprise the amino acid sequences set forth in SEQ ID NO:189 and SEQ ID NO:190, respectively;
the V_{H} region and V_{L} region of the second CAR comprise the amino acid sequences set forth in SEQ ID NO:191 and SEQ ID NO:192;
the V_{H} region and V_{L} region of the second CAR comprise the amino acid sequences set forth in SEQ ID NO:193 and SEQ ID NO:194;
the V_{H} region and V_{L} region of the second CAR comprise the amino acid sequences set forth in SEQ ID NO:195 and SEQ ID NO:196; or
the V_{H} region and V_{L} region of the second CAR comprise the amino acid sequences set forth in SEQ ID NO:197 and SEQ ID NO:198, respectively.

96. The polynucleotide of any of claims 89-95, wherein the V_{H} region of the second CAR is amino-terminal to the V_{L} region.

97. The polynucleotide of any of claims 89-95, wherein the V_{H} region of second CAR is carboxy-terminal to the V_{L} region.

98. The polynucleotide of any of claims 89-97, wherein the antigen-binding domain of the second CAR comprises the amino acid sequence selected from SEQ ID NOs: 237, 238, 239, 240, and 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 237, 238, 239, 240, and 241.

99. The polynucleotide of any of claims 89-98, wherein the antigen-binding domain of the second CAR comprises the amino acid sequence selected from SEQ ID NOs: 237, 238, 239, 240, and 241.

100. The polynucleotide of any of claims 89-99, wherein:
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively; and/or
the V_{H} region and V_{L} region of the second CAR comprises the amino acid sequences set forth in SEQ ID NO:197 and SEQ ID NO:198, respectively; and/or
the extracellular antigen-binding domain of the second CAR comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.

101. The polynucleotide of any of claims 86-100, wherein the transmembrane domain of the second CAR is or comprises a transmembrane domain from CD4, CD28, or CD8, optionally from human CD4, human CD28 or human CD8.

102. The polynucleotide of any of claims 86-101, wherein:
the transmembrane domain of the second CAR is or comprises a transmembrane domain from human CD28; and/or
the transmembrane domain of the second CAR is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.

103. The polynucleotide of any of claims 86-102, wherein the intracellular signaling region of the second CAR comprises an intracellular signaling domain.

104. The polynucleotide of claim 103, wherein the intracellular signaling domain of the second CAR is or comprises a cytoplasmic signaling domain of a CD3-zeta (CD3ζ) chain or a functional variant or signaling portion thereof, optionally a human CD3 zeta chain.

105. The polynucleotide of any of claim 103 or 104, wherein the intracellular signaling region of the second CAR comprises the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.

106. The polynucleotide of any of claims 103-105, wherein the intracellular signaling region of the second CAR further comprises a costimulatory signaling region.

107. The polynucleotide of claim 106, wherein the costimulatory signaling region of the second CAR comprises an intracellular signaling domain of CD28, 4-1BB, or ICOS, or a signaling portion thereof, optionally of human CD28, human 4-1BB, or human ICOS.

108. The polynucleotide of any of claims 80-107, wherein at least one of the first chimeric antigen receptor and the second chimeric antigen receptor comprises an intracellular signaling region comprising an intracellular signaling domain of 4-1BB or a signaling portion thereof, optionally of human 4-1BB.

109. The polynucleotide of any one of claims 106-108, wherein the costimulatory signaling region of the second CAR comprises an intracellular signaling domain of 4-1BB or a signaling portion thereof, optionally human 4-1BB.

110. The polynucleotide of any of claims 106-108, wherein the costimulatory signaling region of the second CAR comprises:
an intracellular signaling domain of human CD28; and/or
the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.

111. The polynucleotide of any of claims 106-109, wherein the costimulatory signaling region of the second CAR comprises:
an intracellular signaling domain of human 4-1BB; and/or
the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.

112. A polynucleotide comprising:
(i) a first nucleic acid sequence encoding a first chimeric antigen receptor (CAR) comprising a first antigen binding domain; and
(ii) a second nucleic acid sequence encoding a second chimeric antigen receptor (CAR) comprising a second antigen binding domain;
wherein the first CAR and second CAR each comprise the following: (a) the first antigen binding domain or the second antigen binding domain, (b) a spacer, (c) a transmembrane domain, and (d) an intracellular signaling region comprising an intracellular signaling domain and a costimulatory signaling region;
wherein one or more of (b) through (d) in the first CAR and the same one or more of (b) through (d) in the second CAR comprises the identical amino acid sequence; and
wherein the nucleotide sequence(s) encoding the one or more of (b) through (d) in the first CAR differs in sequence from the nucleotide sequence(s) encoding the same one or more of (b) through (d) in the second CAR.

113. A polynucleotide comprising:
(i) a first nucleic acid sequence encoding a first chimeric antigen receptor (CAR) comprising a first antigen binding domain capable of binding to one of GPRC5D or BCMA and
(ii) a second nucleic acid sequence encoding a second chimeric antigen receptor (CAR) comprising a second antigen binding domain capable of binding to the other of GPRC5D or BCMA;
wherein the first CAR and second CAR each comprise the following: (a) the first antigen binding domain or the second antigen binding domain, (b) a spacer, (c) a transmembrane domain, and (d) an intracellular signaling region comprising an intracellular signaling domain and a costimulatory signaling region;
wherein one or more of (b) through (d) in the first CAR and the same one or more of (b) through (d) in the second CAR comprises the identical amino acid sequence; and
wherein the nucleotide sequence(s) encoding the one or more of (b) through (d) in the first CAR differs in sequence from the nucleotide sequence(s) encoding the same one or more of (b) through (d) in the second CAR.

114. The polynucleotide of any of claims 80-113, wherein at least one of the nucleotide sequence encoding the first chimeric antigen receptor and the nucleotide sequence encoding the second chimeric antigen receptor is codon diverged.

115. A polynucleotide comprising (i) a first nucleic acid sequence encoding a first chimeric antigen receptor (CAR), (ii) a second nucleic acid sequence encoding a second chimeric antigen receptor (CAR) and (iii) a nucleotide sequence encoding a multicistronic element, wherein the first nucleic acid encoding the first CAR and the second nucleic acid encoding the second CAR are separated by the multicistronic element;
wherein the first CAR comprises a first antigen binding domain that binds to GPRC5D, optionally wherein the first antigen binding domain is encoded by the nucleotide sequence set forth in SEQ ID NO:311; a spacer encoded by the nucleotide set forth in SEQ ID NO:305; a transmembrane domain encoded by the nucleotide sequence set forth in SEQ ID NO:307; and an intracellular signaling region comprising an intracellular signaling domain encoded by the nucleotide sequence set forth in SEQ ID NO:309 and a co-stimulatory signaling region encoded by the nucleotide sequence set forth in SEQ ID NO:308;
wherein the second CAR comprises a second antigen binding domain that binds to BCMA optionally wherein the second antigen binding domain is encoded by the nucleotide sequence set forth in SEQ ID NO:310; a spacer encoded by the nucleotide set forth in SEQ ID NO:74; a transmembrane domain encoded by the nucleotide sequence set forth in SEQ ID NO:56; and an intracellular signaling region comprising an intracellular signaling domain encoded by the nucleotide sequence set forth in SEQ ID NO:58 and a co-stimulatory signaling domain region encoded by the nucleotide sequence set forth in SEQ ID NO:60;
and wherein the first nucleic acid sequence encoding the first CAR is located toward the 5′ end of the polynucleotide relative to the second nucleic acid sequence encoding the second CAR.

116. A polynucleotide comprising (i) a first nucleic acid sequence encoding a first chimeric antigen receptor (CAR), (ii) a second nucleic acid sequence encoding a second chimeric antigen receptor (CAR), and (iii) a nucleotide sequence encoding a multicistronic element, wherein the first nucleic acid encoding the first CAR and the second nucleic acid encoding the second CAR are separated by the multicistronic element;
wherein the first CAR comprises a first antigen binding domain that binds to BCMA, optionally wherein the first antigen binding domain is encoded by the nucleotide sequence set forth in SEQ ID NO:310, a spacer encoded by the nucleotide set forth in SEQ ID NO:74, a transmembrane domain encoded by the nucleotide sequence set forth in SEQ ID NO:56, and an intracellular signaling region comprising an intracellular signaling domain encoded by the nucleotide sequence set forth in SEQ ID NO:58 and a co-stimulatory signaling domain region encoded by the nucleotide sequence set forth in SEQ ID NO:60
wherein the second CAR comprises a second antigen binding domain that binds to GPRC5D, optionally wherein the second antigen binding domain is encoded by the nucleotide sequence set forth in SEQ ID NO:311, a spacer encoded by the nucleotide set forth in SEQ ID NO:305, a transmembrane domain encoded by the nucleotide sequence set forth in SEQ ID NO:307, and an intracellular signaling region comprising an intracellular signaling domain encoded by the nucleotide sequence set forth in SEQ ID NO:309 and a co-stimulatory signaling region encoded by the nucleotide sequence set forth in SEQ ID NO:308;
wherein the first nucleic acid encoding the first CAR is located toward the 5′ end of the polynucleotide relative to the second nucleic acid sequence encoding the second CAR.

117. The polynucleotide of any one of claims 80-116, wherein the nucleotide sequence encoding the first chimeric antigen receptor and the nucleotide sequence encoding the second chimeric antigen receptor have no more than about 30, no more than about 20, or no more than about 10 consecutive base pairs of sequence homology.

118. A vector comprising the polynucleotide of any of claims 68-117.

119. The vector of claim 118, which is a viral vector.

120. A cell comprising the chimeric antigen receptor of any of claims 1-67.

121. The cell of claim 120, wherein the chimeric antigen receptor is a first chimeric receptor and the cell further comprises a polynucleotide comprising a nucleotide encoding a second chimeric antigen receptor.

122. A cell comprising the polynucleotide of any of claims 68-117.

123. A cell comprising the polynucleotide of any of claims 68-79, which is the first polynucleotide, wherein the cell further comprises a second polynucleotide comprising a nucleotide sequence encoding a second chimeric antigen receptor (CAR).

124. The cell of claim 121 or claim 123, wherein the second chimeric antigen receptor (CAR) comprises an extracellular antigen-binding domain that specifically binds a second antigen expressed on or associated with multiple myeloma.

125. The cell of claim 124, wherein the second CAR further comprises a spacer, a transmembrane domain, and an intracellular signaling region.

126. The cell of claim 124 or claim 125, wherein the second antigen is selected from B cell maturation antigen (BCMA), CD38, CD138, CS-1, BAFF-R, TACI, and FcRH5.

127. The cell of any of claims 124-126, wherein the second antigen is BCMA.

128. The cell of any of claims 121-127, wherein the second CAR comprises:
(1) an extracellular antigen-binding domain that specifically binds BCMA, wherein the extracellular antigen-binding domain comprises:
(i) a variable heavy chain (V_{H}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 189, 191, 193, 195 or 197; and
(ii) a variable light chain (V_{L}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 190, 192, 194, 196 or 198;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is at or about 228 amino acids in length, or a spacer set forth in SEQ ID NO:17;
(3) a transmembrane domain; and
(4) an intracellular signaling region.

129. The cell of claim 128, wherein the V_{H} region of the second CAR comprises a CDR-H1, CDR-H2 and CDR-H3 contained within the V_{H} region amino acid sequence set forth in SEQ ID NO: 189, 191, 193, 195 or 197; and the V_{L} region comprises a CDR-L1, CDR-L2 and CDR-L3 contained within the V_{L} region amino acid sequence set forth in SEQ ID NO: 190, 192, 194, 196 or 198.

130. The cell of any of claims 121-127, wherein the second CAR comprises:
(1) an extracellular antigen-binding domain that specifically binds BCMA, wherein the extracellular antigen-binding domain comprises:
(i) a variable heavy chain (V_{H}) region comprising a heavy chain complementarity determining region 1 (CDR-H1) comprising the amino acid sequence set forth in SEQ ID NO: 199, 202, 206, or 209; (b) a heavy chain complementarity determining region 2 (CDR-H2) comprising the amino acid sequence set forth in SEQ ID NO: 200, 203, 207, or 210; and (c) a heavy chain complementarity determining region 3 (CDR-H3) comprising the amino acid sequence set forth in SEQ ID NO: 201, 204, 205, 208, or 211; and
(ii) a variable light chain (V_{L}) region comprising a light chain complementarity determining region 1 (CDR-L1) comprising the amino acid sequence set forth in SEQ ID NO: 218, 221, 224, 227, or 230; (b) a light chain complementarity determining region 2 (CDR-L2) comprising the amino acid sequence set forth in SEQ ID NO: 219, 222, 225, 228, or 231; and (c) a light chain complementarity determining region 3 (CDR-L3) comprising the amino acid sequence set forth in SEQ ID NO: 220, 223, or 226;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length, or a spacer set forth in SEQ ID NO:17;
(3) a transmembrane domain; and
(4) an intracellular signaling region.

131. The cell of any of claims 128-130, wherein:
the V_{H} region of the second CAR comprises a CDR-H1, CDR-H2, and CDR-H3 comprising the amino acid sequence of SEQ ID NOS:199, 200 and 201, respectively, and the V_{L} region of the second CAR comprises a CDR-L1, CDR-L2, and CDR-L3 comprising the amino acid sequence of SEQ ID NOS:218, 219 and 220, respectively;
the V_{H} region of the second CAR comprises a CDR-H1, CDR-H2, and CDR-H3 comprising the amino acid sequence of SEQ ID NOS:202, 203, 204, respectively, and the V_{L} region of the second CAR comprises a CDR-L1, CDR-L2, and CDR-L3 comprising the amino acid sequence of SEQ ID NOS:221, 222 and 223, respectively;
the V_{H} region of the second CAR comprises a CDR-H1, CDR-H2, and CDR-H3 comprising the amino acid sequence of SEQ ID NOS:199, 200, 205, respectively, and the V_{L} region of the second CAR comprises a CDR-L1, CDR-L2, and CDR-L3 comprising the amino acid sequence of SEQ ID NOS:224, 225, and 226, respectively;
the V_{H} region of the second CAR comprises a CDR-H1, CDR-H2, and CDR-H3 comprising the amino acid sequence of SEQ ID NOS:206, 207, 208, respectively, and the V_{L} region of the second CAR comprises a CDR-L1, CDR-L2, and CDR-L3 comprising the amino acid sequence of SEQ ID NOS:227, 228 and 229, respectively; or
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively.

132. The cell of any of claims 128-131, wherein:
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively.

133. The cell of any of claims 128-132, wherein:
the V_{H} region and V_{L} region of the second CAR comprises (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the V_{H} region and V_{L} region of the second CAR comprises (a) the amino acid sequences set forth in SEQ ID NO:191 and SEQ ID NO:192, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the V_{H} region and V_{L} region of the second CAR comprises (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the V_{H} region and V_{L} region of the second CAR comprises (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the V_{H} region and V_{L} region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:198.

134. The cell of any of claims 128-133, wherein:
the V_{H} region and V_{L} region of the second CAR comprise the amino acid sequences set forth in SEQ ID NO:189 and SEQ ID NO:190, respectively;
the V_{H} region and V_{L} region of the second CAR comprise the amino acid sequences set forth in SEQ ID NO:191 and SEQ ID NO:192;
the V_{H} region and V_{L} region of the second CAR comprise the amino acid sequences set forth in SEQ ID NO:193 and SEQ ID NO:194;
the V_{H} region and V_{L} region of the second CAR comprise the amino acid sequences set forth in SEQ ID NO:195 and SEQ ID NO:196; or
the V_{H} region and V_{L} region of the second CAR comprise the amino acid sequences set forth in SEQ ID NO:197 and SEQ ID NO:198, respectively.

135. The cell of claim 128-134, wherein the single chain antibody fragment is or comprises a single chain variable fragment (scFv).

136. The cell of any of claims 128-135, wherein the V_{H} region of the second CAR is amino-terminal to the V_{L} region of the second CAR.

137. The cell of any of claims 128-135 wherein the V_{H} region of the second CAR is carboxy-terminal to the V_{L} region of the second CAR.

138. The cell of any of claims 128-137, wherein the antigen-binding domain of the second CAR comprises the amino acid sequence selected from SEQ ID NOs: 237, 238, 239, 240, and 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 237, 238, 239, 240, and 241.

139. The cell of any of claims 128-138, wherein the antigen-binding domain comprises the amino acid sequence selected from SEQ ID NOs: 237, 238, 239, 240, and 241.

140. The cell of any of claims 128-139, wherein:
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively; and/or
the V_{H} region and V_{L} region of the second CAR comprise the amino acid sequences set forth in SEQ ID NO:197 and SEQ ID NO:198, respectively; and/or
the antigen-binding domain comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.

141. The cell of any of claims 125-140, wherein the transmembrane domain of the second CAR is or comprises a transmembrane domain from CD4, CD28, or CD8, optionally from human CD4, human CD28 or human CD8.

142. The cell of any of claims 125-141, wherein the intracellular signaling region of the second CAR further comprises a costimulatory signaling region.

143. The cell of claim 142, wherein the costimulatory signaling region comprises an intracellular signaling domain of CD28, 4-1BB, or ICOS, or a signaling portion thereof, optionally human CD28, human 4-1BB, or human ICOS.

144. The cell of claim 142 or claim 143, wherein the costimulatory signaling region comprises an intracellular signaling domain of 4-1BB or a signaling portion thereof, optionally human 4-1BB.

145. A composition comprising the chimeric antigen receptor of any of claims 1-67.

146. A composition comprising the cell of any one of claims 120-144 or a plurality of the cells of any one of claims 120-144.

147. The composition of claim 143, wherein the composition comprises CD4+ and CD8+ T cells and the ratio of CD4+ to CD8+ T cells is from about 1:3 to 3:1, optionally about 1:2 to 2:1.

148. A composition comprising:
a plurality of first cells comprising a first chimeric antigen receptor that is the chimeric antigen receptor of any of claims 1-67 or encoded by the polynucleotide of any of claims 68-79; and
a plurality of second cells comprising a second chimeric antigen receptor.

149. The composition of claim 148, wherein the second chimeric receptor comprises an extracellular antigen-binding domain that specifically binds a second antigen expressed on or associated with multiple myeloma.

150. The composition of claim 148 or claim 149, wherein the second CAR comprises the extracellular antigen-binding domain that binds the second antigen, a spacer, a transmembrane domain, and an intracellular signaling region.

151. The composition of claim 149 or claim 150, wherein the second antigen is selected from B cell maturation antigen (BCMA), CD38, CD138, CS-1, BAFF-R, TACI, and FcRH5.

152. The composition of any of claims 149-151, wherein the second antigen is BCMA.

153. The composition of any of claims 148-152, wherein the second CAR comprises:
(1) an extracellular antigen-binding domain that specifically binds BCMA, wherein the extracellular antigen-binding domain comprises:
(i) a variable heavy chain (V_{H}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 189, 191, 193, 195 or 197; and
(ii) a variable light chain (V_{L}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 190, 192, 194, 196 or 198;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length, or a spacer set forth in SEQ ID NO:17;
(3) a transmembrane domain; and
(4) an intracellular signaling region.

154. The composition of claim 153, wherein the V_{H} region of the second CAR comprises a CDR-H1, CDR-H2 and CDR-H3 contained within the V_{H} region amino acid sequence set forth in SEQ ID NO: 189, 191, 193, 195 or 197; and the V_{L} region comprises a CDR-L1, CDR-L2 and CDR-L3 contained within the V_{L} region amino acid sequence set forth in SEQ ID NO: 190, 192, 194, 196, or 198.

155. The composition of any of claims 148-152, wherein the second CAR comprises:
(1) an extracellular antigen-binding domain that specifically binds BCMA, wherein the extracellular antigen-binding domain comprises:
(i) a variable heavy chain (V_{H}) region comprising a heavy chain complementarity determining region 1 (CDR-H1) comprising the amino acid sequence set forth in SEQ ID NO: 199, 202, 206, or 209; (b) a heavy chain complementarity determining region 2 (CDR-H2) comprising the amino acid sequence set forth in SEQ ID NO: 200, 203, 207, or 210; and (c) a heavy chain complementarity determining region 3 (CDR-H3) comprising the amino acid sequence set forth in SEQ ID NO: 201, 204, 205, 208, or 211; and
(ii) a variable light chain (V_{L}) region comprising a light chain complementarity determining region 1 (CDR-L1) comprising the amino acid sequence set forth in SEQ ID NO: 218, 221, 224, 227, or 230; (b) a light chain complementarity determining region 2 (CDR-L2) comprising the amino acid sequence set forth in SEQ ID NO: 219, 222, 225, 228, or 231; and (c) a light chain complementarity determining region 3 (CDR-L3) comprising the amino acid sequence set forth in SEQ ID NO: 220, 223, or 226;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length, or a spacer set forth in SEQ ID NO:17;
(3) a transmembrane domain; and
(4) an intracellular signaling region.

156. The composition of any of claims 153-155, wherein:
the V_{H} region of the second CAR comprises a CDR-H1, CDR-H2, and CDR-H3 comprising the amino acid sequence of SEQ ID NOS:199, 200 and 201, respectively, and the V_{L} region of the second CAR comprises a CDR-L1, CDR-L2, and CDR-L3 comprising the amino acid sequence of SEQ ID NOS:218, 219 and 220, respectively;
the V_{H} region of the second CAR comprise a CDR-H1, CDR-H2, and CDR-H3 comprising the amino acid sequence of SEQ ID NOS:202, 203, 204, respectively, and the V_{L} region of the second CAR comprises a CDR-L1, CDR-L2, and CDR-L3 comprising the amino acid sequence of SEQ ID NOS:221, 222 and 223, respectively;
the V_{H} region of the second CAR comprises a CDR-H1, CDR-H2, and CDR-H3 comprising the amino acid sequence of SEQ ID NOS:199, 200, 205, respectively, and the V_{L} region of the second CAR comprises a CDR-L1, CDR-L2, and CDR-L3 comprising the amino acid sequence of SEQ ID NOS:224, 225, and 226, respectively;
the V_{H} region of the second CAR comprises a CDR-H1, CDR-H2, and CDR-H3 comprising the amino acid sequence of SEQ ID NOS:206, 207, 208, respectively, and the V_{L} region of the second CAR comprises a CDR-L1, CDR-L2, and CDR-L3 comprising the amino acid sequence of SEQ ID NOS:227, 228 and 229, respectively; or
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively.

157. The composition of any of claims 153-156, wherein:
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively.

158. The composition of any of claims 153-157, wherein:
the V_{H} region and V_{L} region of the second CAR comprise (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the V_{H} region and V_{L} region of the second CAR comprises (a) the amino acid sequences set forth in SEQ ID NO:191 and SEQ ID NO:192, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the V_{H} region and V_{L} region of the second CAR comprises (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the V_{H} region and V_{L} region of the second CAR comprises (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the V_{H} region and V_{L} region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:198.

159. The composition of any of claims 153-158, wherein:
the V_{H} region and V_{L} region of the second CAR comprise the amino acid sequences set forth in SEQ ID NO:189 and SEQ ID NO:190, respectively;
the V_{H} region and V_{L} region of the second CAR comprise the amino acid sequences set forth in SEQ ID NO:191 and SEQ ID NO:192;
the V_{H} region and V_{L} region of the second CAR comprise the amino acid sequences set forth in SEQ ID NO:193 and SEQ ID NO:194;
the V_{H} region and V_{L} region of the second CAR comprise the amino acid sequences set forth in SEQ ID NO:195 and SEQ ID NO:196; or
the V_{H} region and V_{L} region of the second CAR comprise the amino acid sequences set forth in SEQ ID NO:197 and SEQ ID NO:198, respectively.

160. The composition of any of claims 153-159, wherein the V_{H} region of the second CAR is amino-terminal to the V_{L} region of the second CAR.

161. The composition of any of claims 153-159, wherein the V_{H} region of the second CAR is carboxy-terminal to the V_{L} region of the second CAR.

162. The composition of any of claims 153-161, wherein the antigen-binding domain of the second CAR comprises the amino acid sequence selected from SEQ ID NOs: 237, 238, 239, 240, and 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 237, 238, 239, 240, and 241.

163. The composition of any of claims 153-162, wherein the antigen-binding domain of the second CAR comprises the amino acid sequence selected from SEQ ID NOs: 237, 238, 239, 240, and 241.

164. The composition of any of claims 153-163, wherein:
the V_{H} region of the second CAR comprises a CDR-H1, CDR-H2, and CDR-H3 comprising the amino acid sequence of SEQ ID NOS:209, 210 and 211, respectively, and the V_{L} region of the second CAR comprises a CDR-L1, CDR-L2, and CDR-L3 comprising the amino acid sequence of SEQ ID NOS:230, 231 and 232, respectively; and/or
the V_{H} region and V_{L} region of the second CAR comprise the amino acid sequences set forth in SEQ ID NO:197 and SEQ ID NO:198, respectively; and/or
the antigen-binding domain comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.

165. The composition of any of claims 150-164, wherein the transmembrane domain of the second CAR is or comprises a transmembrane domain from CD4, CD28, or CD8, optionally from human CD4, human CD28 or human CD8.

166. The composition of any of claims 150-165, wherein:
the transmembrane domain of the second CAR is or comprises a transmembrane domain from human CD28; and/or
the transmembrane domain of the second CAR is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.

167. The composition of any of claims 150-166, wherein the intracellular signaling region comprises an intracellular signaling domain, wherein the intracellular signaling domain is capable of inducing a primary activation signal in a T cell, is a T cell receptor (TCR) component and/or comprises an immunoreceptor tyrosine-based activation motif (ITAM).

168. The composition of claim 167, wherein the intracellular signaling domain is or comprises a cytoplasmic signaling domain of a CD3-zeta (CD3ζ) chain or a functional variant or signaling portion thereof, optionally a human CD3 zeta chain.

169. The composition of any of claims 150-168, wherein the intracellular signaling region of the second CAR further comprises a costimulatory signaling region.

170. The composition of claim 169, wherein the costimulatory signaling region of the second CAR comprises an intracellular signaling domain of CD28, 4-1BB, or ICOS, or a signaling portion thereof, optionally human CD28, human 4-1BB, or human ICOS.

171. The composition of any of claims 148-170, wherein at least one of the first chimeric antigen receptor and the second chimeric antigen receptor comprises an intracellular signaling region comprising an intracellular signaling domain of 4-1BB or a signaling portion thereof, optionally of human 4-1BB.

172. The composition of any one of claims 169-171, wherein the costimulatory signaling region of the second CAR comprises an intracellular signaling domain of 4-1BB or a signaling portion thereof, optionally human 4-1BB.

173. The composition of any of claims 148-172, wherein the plurality of first cells comprises T cells, optionally wherein the T cells comprises CD4+ and CD8+ T cells, optionally wherein the ratio of CD4+ to CD8+ T cells is from about 1:3 to 3:1, optionally 1:2 to 2:1.

174. The composition of any of claims 148-173, wherein the plurality of second cells comprises T cells, optionally wherein the T cells comprises CD4+ and CD8+ T cells, optionally wherein the ratio of CD4+ to CD8+ T cells is from about 1:3 to 3:1, optionally about 1:2 to 2:1.

175. The composition of any of claims 148-174, wherein the ratio of the first plurality of cells and the second plurality of cells in the composition is from about 1:3 to 3:1, optionally about 1:2 to 2:1, optionally about 1:1.

176. The composition of any of claims 148-175, wherein the composition comprises the first plurality of cells expressing the first chimeric antigen receptor and the second plurality of cells expressing the second chimeric antigen receptor at a ratio that is from about 1:3 to 3:1, optionally about 1:2 to 2:1, optionally about 1:1.

177. A pharmaceutical composition of any of claims 145-176, for use in treating a subject with a disease or condition, optionally wherein the disease or condition is a cancer.

178. A pharmaceutical composition for use in treating a disease or disorder, optionally a cancer, containing the cells of any of claims 120-144 as an active ingredient.

179. A pharmaceutical composition for use treating a disease or disorder, optionally a cancer, containing the composition of any of claim 145-176, 233 or 234 as an active ingredient.

180. A pharmaceutical composition for use in treating a disease or disorder, optionally a cancer, containing a composition comprising a first dose of a plurality of first cells comprising a first chimeric antigen receptor that is the chimeric antigen receptor of any of claims 1-67 or encoded by the polynucleotide of any of claims 68-79 and a composition comprising a second dose of a plurality of second cells comprising a second chimeric antigen receptor as an active ingredient.

181. A method of treatment, comprising administering a composition comprising a dose of cells of any of claims 120-144 or the composition of any of claim 145-180, 233 or 234 to a subject having a disease or disorder.

182. Use of the cells of any of claims 120-144 for treatment of a disease or disorder, optionally wherein the disease or condition is a cancer.

183. Use of the composition of any of claim 145-180, 233 or 234 for treatment of a disease or disorder, optionally wherein the disease or condition is a cancer.

184. The use of the cells of any of claims 120-144 for the manufacture of a medicament for treatment of a disease or disorder, optionally wherein the disease or condition is a cancer.

185. The use of the composition of any of claim 145-180, 233 or 234 for the manufacture of a medicament for treatment of a disease or disorder, optionally wherein the disease or condition is a cancer.

186. The method or use of any of claims 181-185 or the pharmaceutical composition for use of any of claims 177-180, wherein the dose of cells comprises between about 1.0×10⁷ CAR-expressing T cells and 1.2×10⁹ CAR-expressing T cells, between about 1.0×10⁷ CAR-expressing T cells and 6.5×10⁸ CAR-expressing T cells, between about 1.5×10⁷ CAR-expressing T cells and 6.5×10⁸ CAR-expressing T cells, between about 1.5×10⁷ CAR-expressing T cells and 6.0×10⁸ CAR-expressing T cells, between about 2.5×10⁷ CAR-expressing T cells and 6.0×10⁸ CAR-expressing T cells, between about 5.0×10⁷ CAR-expressing T cells and 6.0×10⁸ CAR-expressing T cells, between about 1.25×10⁷ CAR-expressing T cells and 1.2×10⁹ CAR-expressing T cells, between about 1.5×10⁷ CAR-expressing T cells and 1.2×10⁹ CAR-expressing T cells, between about 5.0×10⁷ CAR-expressing T cells and 4.5×10⁸ CAR-expressing T cells, or between about 1.5×10⁸ CAR-expressing T cells and 3.0×10⁸ CAR-expressing T cells, each inclusive.

187. The method or use of any of claims 181-186 or the pharmaceutical composition for use of any of claims 177-180, wherein the dose of cells comprises at or about 1.5×10⁷, at or about 2.5×10⁷, at or about 5.0×10⁷, at or about 7.5×10⁷, at or about 1.5×10⁸, at or about 2.25×10⁸, at or about 3.0×10⁸, at or about 4.5×10⁸, at or about 6.0×10⁸, at or about 8.0×10⁸, or at or about 1.2×10⁹ CAR-expressing T cells.

188. A method of treatment, comprising:
administering a composition comprising a first dose of a plurality of first cells comprising a first chimeric antigen receptor that is the chimeric antigen receptor of any of claims 1-67 or encoded by the polynucleotide of any of claims 68-79 to a subject having a disease or disorder; and
administering to the subject a composition comprising a second dose of a plurality of second cells comprising a second chimeric antigen receptor.

189. Use of a composition comprising a first dose of a plurality of first cells comprising a first chimeric antigen receptor that is the chimeric antigen receptor of any of claims 1-67 or encoded by the polynucleotide of any of claims 68-79 and a composition comprising a second dose of a plurality of second cells comprising a second chimeric antigen receptor for treatment of a disease or disorder, optionally wherein the disease or condition is a cancer.

190. The use of a composition comprising a first dose of a plurality of first cells comprising a first chimeric antigen receptor that is the chimeric antigen receptor of any of claims 1-67 or encoded by the polynucleotide of any of claims 68-79 and a composition comprising a second dose of a plurality of second cells comprising a second chimeric antigen receptor for the manufacture of a medicament for treatment of a disease or disorder, optionally wherein the disease or condition is a cancer.

191. The method or use of any of claims 188-190, wherein the first dose of the plurality of first cells and the second dose of the plurality of second cells independently comprise between at or about 1.0×10⁷ CAR-expressing T cells and at or about 1.5×10⁹ CAR-expressing T cells, between at or about about 1.0×10⁷ CAR-expressing T cells and at or about 6.5×10⁸ CAR-expressing T cells, between at or about 1.25×10⁷ CAR-expressing T cells and at or about 0.6×10⁸ CAR-expressing T cells, between at or about 1.5×10⁷ CAR-expressing T cells and at or about 6.5×10⁸ CAR-expressing T cells, between at or about 1.5×10⁷ CAR-expressing T cells and at or about 6.0×10⁸ CAR-expressing T cells, between at or about 2.5×10⁷ CAR-expressing T cells and at or about 2.25×10⁸ CAR-expressing T cells, between at or about 2.5×10⁷ CAR-expressing T cells and at or about 6.0×10⁸ CAR-expressing T cells, between at or about 5.0×10⁷ CAR-expressing T cells and at or about 6.0×10⁸ CAR-expressing T cells, between at or about 7.5×10⁷ CAR-expressing T cells and at or about 1.5×10⁸ CAR-expressing T cells, between at or about 2.5×10⁷ CAR-expressing T cells and at or about 1.2×10⁹ CAR-expressing T cells, between at or about 5.0×10⁷ CAR-expressing T cells and at or about 4.5×10⁸ CAR-expressing T cells, or between at or about 1.5×10⁸ CAR-expressing T cells and at or about 3.0×10⁸ CAR-expressing T cells, each inclusive.

192. The method or use of any of claims 188-190 or the pharmaceutical composition for use of claim 180, wherein the second chimeric receptor comprises an extracellular antigen binding domain that specifically binds a second antigen expressed on or associated with multiple myeloma.

193. The method or use or pharmaceutical composition for use of claim 192, wherein the second antigen is selected from B cell maturation antigen (BCMA), CD38, CD138, CS-1, BAFF-R, TACI, and FcRH5.

194. The method or use or pharmaceutical composition for use of any of claims 188-193, wherein the second antigen is BCMA.

195. The method or use or pharmaceutical composition for use of any of claims 188-194, wherein the second CAR comprises:
(1) an extracellular antigen-binding domain that specifically binds BCMA, wherein the extracellular antigen-binding domain comprises:
(i) a variable heavy chain (V_{H}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 189, 191, 193, 195 or 197; and
(ii) a variable light chain (V_{L}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 190, 192, 194, 196 or 198;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length, or a spacer set forth in SEQ ID NO:17;
(3) a transmembrane domain; and
(4) an intracellular signaling region.

196. The method or use or pharmaceutical composition for use of claim 195, wherein the V_{H} region of the second CAR comprises a CDR-H1, CDR-H2 and CDR-H3 contained within the V_{H} region amino acid sequence set forth in SEQ ID NO: 189, 191, 193, 195 or 197; and the V_{L} region comprises a CDR-L1, CDR-L2 and CDR-L3 contained within the V_{L} region amino acid sequence set forth in SEQ ID NO: 190, 192, 194, 196 or 198.

197. The method or use or pharmaceutical composition for use of any of claims 188-196, wherein the second CAR comprises:
(1) an extracellular antigen-binding domain that specifically binds BCMA, wherein the extracellular antigen-binding domain comprises:
(i) a variable heavy chain (V_{H}) region comprising a heavy chain complementarity determining region 1 (CDR-H1) comprising the amino acid sequence selected from SEQ ID NOs: 199, 202, 206, and 209; (b) a heavy chain complementarity determining region 2 (CDR-H2) comprising the amino acid sequence selected from SEQ ID NOs: 200, 203, 207, and 210; and (c) a heavy chain complementarity determining region 3 (CDR-H3) comprising the amino acid sequence selected from SEQ ID NOs: 201, 204, 205, 208, and 211; and
(ii) a variable light chain (V_{L}) region comprising a light chain complementarity determining region 1 (CDR-L1) comprising the amino acid sequence selected from SEQ ID NOs: 218, 221, 224, 227, and 230; (b) a light chain complementarity determining region 2 (CDR-L2) comprising the amino acid sequence selected from SEQ ID NOs: 219, 222, 225, 228, and 231, 234; and (c) a light chain complementarity determining region 3 (CDR-L3) comprising the amino acid sequence selected from SEQ ID NOs: 220, 223, and 226;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length, or a spacer set forth in SEQ ID NO:17;
(3) a transmembrane domain; and
(4) an intracellular signaling region.

198. The method or use or pharmaceutical composition for use of any of claims 195-197, wherein:
the V_{H} region of the second CAR comprises a CDR-H1, CDR-H2, and CDR-H3 comprising the amino acid sequences of SEQ ID NOS:199, 200 and 201, respectively, and the V_{L} region of the second CAR comprises a CDR-L1, CDR-L2, and CDR-L3 comprising the amino acid sequences of SEQ ID NOS:218, 219 and 220, respectively;
the V_{H} region of the second CAR comprises a CDR-H1, CDR-H2, and CDR-H3 comprising the amino acid sequences of SEQ ID NOS:202, 203, 204, respectively, and the V_{L} region of the second CAR comprises a CDR-L1, CDR-L2, and CDR-L3 comprising the amino acid sequences of SEQ ID NOS:221, 222 and 223, respectively;
the V_{H} region of the second CAR comprises a CDR-H1, CDR-H2, and CDR-H3 comprising the amino acid sequences of SEQ ID NOS:199, 200, 205, respectively, and the V_{L} region of the second CAR comprises a CDR-L1, CDR-L2, and CDR-L3 comprising the amino acid sequences of SEQ ID NOS:224, 225, and 226, respectively;
the V_{H} region of the second CAR comprises a CDR-H1, CDR-H2, and CDR-H3 comprising the amino acid sequences of SEQ ID NOS:206, 207, 208, respectively, and the V_{L} region of the second CAR comprises a CDR-L1, CDR-L2, and CDR-L3 comprising the amino acid sequences of SEQ ID NOS:227, 228 and 229, respectively; or
the V_{H} region of the second CAR comprises a CDR-H1, CDR-H2, and CDR-H3 comprising the amino acid sequences of SEQ ID NOS:209, 210 and 211, respectively, and the V_{L} region of the second CAR comprises a CDR-L1, CDR-L2, and CDR-L3 comprising the amino acid sequences of SEQ ID NOS:230, 231 and 232, respectively.

199. The method or use or pharmaceutical composition for use of any of claims 195-198, wherein:
the V_{H} region of the second CAR comprises a CDR-H1, CDR-H2, and CDR-H3 comprising the amino acid sequences of SEQ ID NOS:209, 210 and 211, respectively, and the V_{L} region of the second CAR comprises a CDR-L1, CDR-L2, and CDR-L3 comprising the amino acid sequences of SEQ ID NOS:230, 231 and 232, respectively.

200. The method or use or pharmaceutical composition for use of any of claims 195-199, wherein:
the V_{H} region and V_{L} region of the second CAR comprise (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the V_{H} region and V_{L} region of the second CAR comprise (a) the amino acid sequences set forth in SEQ ID NO:191 and SEQ ID NO:192, respectively, or (b) an amino acid sequence having a at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the V_{H} region and V_{L} region of the second CAR comprise (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the V_{H} region and V_{L} region of the second CAR comprises (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the V_{H} region and V_{L} region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and SEQ ID NO:198.

201. The method or use or pharmaceutical composition for use of any of claims 195-200, wherein:
the V_{H} region and V_{L} region of the second CAR comprise the amino acid sequences set forth in SEQ ID NO:189 and SEQ ID NO:190, respectively;
the V_{H} region and V_{L} region of the second CAR comprise the amino acid sequences set forth in SEQ ID NO:191 and SEQ ID NO:192;
the V_{H} region and V_{L} region of the second CAR comprise the amino acid sequences set forth in SEQ ID NO:193 and SEQ ID NO:194;
the V_{H} region and V_{L} region of the second CAR comprise the amino acid sequences set forth in SEQ ID NO:195 and SEQ ID NO:196; or
the V_{H} region and V_{L} region of the second CAR comprise the amino acid sequences set forth in SEQ ID NO:197 and SEQ ID NO:198, respectively.

202. The method or use or pharmaceutical composition for use of any of claims 195-201, wherein the V_{H} region of the second CAR is carboxy-terminal to the V_{L} region.

203. The method or use or pharmaceutical composition for use of any of claims 195-202, wherein the antigen-binding domain of the second CAR comprises the amino acid sequence selected from SEQ ID NOs: 237, 238, 239, 240, and 241 or an amino acid sequence having a at least at or about 90%, at or about 91%, at or about 92%, at or about 93%, at or about 94%, at or about 95%, at or about 96%, at or about 97%, at or about 98%, or at or about 99% sequence identity to the amino acid sequence selected from SEQ ID NOs: 237, 238, 239, 240, and 241.

204. The method or use or pharmaceutical composition for use of any of claims 195-203, wherein the antigen-binding domain of the second CAR comprises the amino acid sequence selected from SEQ ID NOs: 237, 238, 239, 240, and 241.

205. The method or use or pharmaceutical composition for use of any of claims 195-204, wherein:
the V_{H} region of the second CAR comprises a CDR-H1, CDR-H2, and CDR-H3 comprising the amino acid sequences of SEQ ID NOS:209, 210 and 211, respectively, and the V_{L} region of the second CAR comprises a CDR-L1, CDR-L2, and CDR-L3 comprising the amino acid sequences of SEQ ID NOS:230, 231 and 232, respectively; and/or
the V_{H} region and V_{L} region of the second CAR comprise the amino acid sequences set forth in SEQ ID NO:197 and SEQ ID NO:198, respectively; and/or
the antigen-binding domain of the second CAR comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.

206. The method or use or pharmaceutical composition for use of any of claims 188-205, wherein at least one of the first chimeric antigen receptor and the second chimeric antigen receptor comprises an intracellular signaling region comprising an intracellular signaling domain of 4-1BB or a signaling portion thereof, optionally of human 4-1BB.

207. The method or use or pharmaceutical composition for use of any of claims 195-206, wherein the transmembrane domain of the second CAR is or comprises a transmembrane domain from CD4, CD28, or CD8, optionally from human CD4, human CD38 or human CD8.

208. The method or use or pharmaceutical composition for use of any of claims 195-207, wherein:
the transmembrane domain of the second CAR is or comprises a transmembrane domain from human CD28; and/or
the transmembrane domain of the second CAR is or comprises the amino acid sequence set forth in SEQ ID NO:18 or an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:18.

209. The method or use or pharmaceutical composition for use of any of claims 195-208, wherein the intracellular signaling region of the second CAR comprises an intracellular signaling domain.

210. The method or use or pharmaceutical composition for use of claim 209, wherein the intracellular signaling domain of the second CAR is or comprises a cytoplasmic signaling domain of a CD3-zeta (CD3ζ) chain or a functional variant or signaling portion thereof, optionally a human CD3 zeta chain.

211. The method or use or pharmaceutical composition for use of any of claim 209 or 210, wherein the intracellular signaling region of the second CAR comprises the amino acid sequence set forth in SEQ ID NO:20 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to SEQ ID NO:20.

212. The method or use or pharmaceutical composition for use of any of claims 209-211, wherein the intracellular signaling region of the second CAR further comprises a costimulatory signaling region.

213. The method or use or pharmaceutical composition for use of claim 212, wherein the costimulatory signaling region of the second CAR comprises an intracellular signaling domain of CD28, 4-1BB, or ICOS or a signaling portion thereof, optionally human CD28, human 4-1BB, or human ICOS.

214. The method or use or pharmaceutical composition for use of claim 212 or claim 213, wherein the costimulatory signaling region of the second CAR comprises an intracellular signaling domain of 4-1BB or a signaling portion thereof, optionally human 4-1BB.

215. The method or use or pharmaceutical composition for use of claim 212 or claim 213, wherein the costimulatory signaling region of the second CAR comprises:
an intracellular signaling domain of human CD28; and/or
the amino acid sequence set forth in SEQ ID NO:46 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 46.

216. The method or use or pharmaceutical composition for use of any of claims 212-214, wherein the costimulatory signaling region of the second CAR comprises:
an intracellular signaling domain of a human 4-1BB; and/or
the amino acid sequence set forth in SEQ ID NO:19 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO: 19.

217. The method or use or pharmaceutical composition for use of any of claims 181-216, wherein the disease or disorder is associated with expression of GPRC5D.

218. The method or use or pharmaceutical composition for use of claim 217, wherein the disease or disorder is further associated with expression of B cell maturation antigen (BCMA).

219. The method or use or pharmaceutical composition for use of any of claims 181-218, wherein the disease or disorder is a B cell-related disorder.

220. The method or use or pharmaceutical composition for use of any one of claims 181-219, wherein the disease or disorder associated with BCMA is an autoimmune disease or disorder.

221. The method or use or pharmaceutical composition for use of claim 220, wherein the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

222. The method or use or pharmaceutical composition for use of any one of claims 181-221, wherein the disease or disorder is a cancer.

223. The method or use or pharmaceutical composition for use of claim 222, wherein the cancer is a GPRC5D-expressing cancer.

224. The method or use or pharmaceutical composition for use of claim 222 or claim 223, wherein the cancer is a plasma cell malignancy and the plasma cell malignancy is multiple myeloma (MM) or plasmacytoma.

225. The method or use or pharmaceutical composition for use of any of any of claims 222-224, wherein the cancer is multiple myeloma (MM).

226. The method or use or pharmaceutical composition for use of claim 225, wherein the cancer is a relapsed/refractory multiple myeloma.

227. The method or use or pharmaceutical composition for use of any of claims 181-226, wherein:
the subject is refractory to or has relapsed following administration of a BCMA-targeted therapy, optionally following administration of T cells comprising a CAR that specifically binds BCMA; or
the method comprises selecting a subject for treatment that is refractory to or has relapsed following administration of a BCMA-targeted therapy, optionally following administration T cells comprising a CAR that specifically binds BCMA.

228. The method or use or pharmaceutical composition for use of any of claims 181-187 and 191-227, wherein prior to the administration of the dose of cells, the subject has previously received administration of a BCMA-targeted therapy for treating the disease or disorder.

229. The method or use or pharmaceutical composition for use of any of claims 188-228, wherein prior to the administration of the first dose of cells and the second dose of cells, the subject has previously received administration of a BCMA-targeted therapy for treating the disease or disorder.

230. The method or use or pharmaceutical composition for use of claim 228 or claim 229, wherein the BCMA-targeted therapy comprises a composition comprising T cells comprising a CAR that specifically binds BCMA.

231. The method or use or pharmaceutical composition for use of any of claims 228-230, wherein the subject is refractory to or has relapsed following administration of the BCMA-targeted therapy, optionally following administration of T cells comprising a CAR that specifically binds BCMA.

232. The method or use or pharmaceutical composition for use of any of claims 181-231, wherein the subject comprises multiple myeloma cells exhibiting BCMA antigen or epitope loss, BCMA downregulation and/or BCMA-negative tumor cells following a previous administration.

233. The composition of claim 146 or claim 147, wherein the composition comprises a plurality of cells, wherein at least a portion of the cells comprise the first CAR that specifically binds GPRC5D, a portion of the cells comprise a second CAR that specifically binds a second antigen that is expressed on or associated with multiple myeloma, optionally wherein the second antigen is BCMA, and a portion of the cells comprise both the first CAR and the second CAR.

234. The composition of claim 233, wherein the ratio of cells expressing the first CAR to cells expressing the second CAR is from or from about 1:3 to 3:1, optionally 1:2 to 2:1, optionally is or is about 1:1.

235. A combination comprising:
a plurality of first cells comprising a first chimeric antigen receptor that is the chimeric antigen receptor of any of claims 1-67 and/or encoded by the polynucleotide of any of claims 68-79; and
a plurality of second cells comprising a second chimeric antigen receptor.

236. The combination of claim 235, wherein the second chimeric receptor comprises an extracellular antigen binding domain that specifically binds a second antigen expressed on or associated with multiple myeloma.

237. The combination of claim 236, wherein the second antigen is selected from the group consisting of B cell maturation antigen (BCMA), CD38, CD138, CS-1, BAFF-R, TACI, and FcRH5.

238. The combination of claim 236 or claim 237, wherein the second antigen is BCMA.

239. The combination of any of claims 235-238, wherein the second CAR comprises:
(1) an extracellular antigen-binding domain that specifically binds BCMA, wherein the extracellular antigen-binding domain comprises:
(i) a variable heavy chain (V_{H}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 189, 191, 193, 195 or 197; and
(ii) a variable light chain (V_{L}) region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in any of SEQ ID NO: 190, 192, 194, 196 or 198;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length and/or a spacer set forth in SEQ ID NO:17;
(3) a transmembrane domain; and
(4) an intracellular signaling region.

240. The combination of claim 239, wherein the V_{H} region comprises a CDR-H1, CDR-H2 and CDR-H3 contained within the V_{H} region amino acid sequence set forth in SEQ ID NO: 189, 191, 193, 195 or 197; and the V_{L} region comprises a CDR-L1, CDR-L2 and CDR-L3 contained within the V_{L} region amino acid sequence set forth in SEQ ID NO: 190, 192, 194, 196 or 198.

241. The combination of any of claims 235-240, wherein the second CAR comprises:
(1) an extracellular antigen-binding domain that specifically binds BCMA, wherein the extracellular antigen-binding domain comprises:
(i) a variable heavy chain (V_{H}) comprising a heavy chain complementarity determining region 1 (CDR-H1) comprising the amino acid sequence set forth in SEQ ID NO: 199, 202, 206, or 209; (b) a heavy chain complementarity determining region 2 (CDR-H2) comprising the amino acid sequence set forth in SEQ ID NO: 200, 203, 207, or 210; and (c) a heavy chain complementarity determining region 3 (CDR-H3) comprising the amino acid sequence set forth in SEQ ID NOs: 201, 204, 205, 208, or 211; and
(ii) a variable light chain (V_{L}) region comprising a light chain complementarity determining region 1 (CDR-L1) comprising the amino acid sequence set forth in SEQ ID NOs: 218, 221, 224, 227, 230, 233, or 235; (b) a light chain complementarity determining region 2 (CDR-L2) comprising the amino acid sequence set forth in SEQ ID NOs: 219, 222, 225, 228, or 231; and (c) a light chain complementarity determining region 3 (CDR-L3) comprising the amino acid sequence set forth in SEQ ID NOs: 220, 223, or 226;
(2) a spacer, optionally a spacer comprising an IgG4/2 chimeric hinge or a modified IgG4 hinge; an IgG2/4 chimeric C_{H}2 region; and an IgG4 C_{H}3 region, optionally that is about 228 amino acids in length and/or a spacer set forth in SEQ ID NO:17;
(3) a transmembrane domain; and
(4) an intracellular signaling region.

242. The combination of any of claims 239-241 wherein:
the V_{H} region of the second CAR comprises a CDR-H1, CDR-H2, and CDR-H3 comprising the amino acid sequences of SEQ ID NOS:199, 200 and 201, respectively, and the V_{L} region of the second CAR comprises a CDR-L1, CDR-L2, and CDR-L3 comprising the amino acid sequences of SEQ ID NOS:218, 219 and 220, respectively;
the V_{H} region of the second CAR comprises a CDR-H1, CDR-H2, and CDR-H3 comprising the amino acid sequence of SEQ ID NOS:202, 203, 204, respectively, and the V_{L} region of the second CAR comprises a CDR-L1, CDR-L2, and CDR-L3 comprising the amino acid sequence of SEQ ID NOS:221, 222 and 223, respectively;
the V_{H} region of the second CAR comprises a CDR-H1, CDR-H2, and CDR-H3 comprising the amino acid sequences of SEQ ID NOS:199, 200, 205, respectively, and the V_{L} region of the second CAR comprises a CDR-L1, CDR-L2, and CDR-L3 comprising the amino acid sequences of SEQ ID NOS:224, 225, and 226, respectively;
the V_{H} region of the second CAR comprises a CDR-H1, CDR-H2, and CDR-H3 comprising the amino acid sequences of SEQ ID NOS:206, 207, 208, respectively, and the V_{L} region of the second CAR comprises a CDR-L1, CDR-L2, and CDR-L3 comprising the amino acid sequences of SEQ ID NOS:227, 228 and 229, respectively; or
the V_{H} region of the second CAR comprises a CDR-H1, CDR-H2, and CDR-H3 comprising the amino acid sequences of SEQ ID NOS:209, 210 and 211, respectively, and the V_{L} region of the second CAR comprises a CDR-L1, CDR-L2, and CDR-L3 comprising the amino acid sequences of SEQ ID NOS:230, 231 and 232, respectively.

243. The combination of any of claims 239-242, wherein:
the V_{H} region of the second CAR comprises a CDR-H1, CDR-H2, and CDR-H3 comprising the amino acid sequences of SEQ ID NOS:209, 210 and 211, respectively, and the V_{L} region of the second CAR comprises a CDR-L1, CDR-L2, and CDR-L3 comprising the amino acid sequences of SEQ ID NOS:230, 231 and 232, respectively.

244. The combination of any of claims 240-243, wherein:
the V_{H} region and V_{L} region of the second CAR comprise (a) the amino acid sequences set forth in SEQ ID NO:189 and SEQ ID NO:190, respectively, or (b) an amino acid sequence having at least at or about 90%, at or about 91%, at or about 92%, at or about 93%, at or about 94%, at or about 95%, at or about 96%, at or about 97%, at or about 98%, at or about 99%, or at or about 100% sequence identity to SEQ ID NO:189 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:190;
the V_{H} region and V_{L} region of the second CAR comprise (a) the amino acid sequences set forth in SEQ ID NO:191 and SEQ ID NO:192, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:191 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:192;
the V_{H} region and V_{L} region of the second CAR comprises (a) the amino acid sequences set forth in SEQ ID NO:193 and SEQ ID NO:194, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:193 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:194;
the V_{H} region and V_{L} region of the second CAR comprises (a) the amino acid sequences set forth in SEQ ID NO:195 and SEQ ID NO:196, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:195 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:196; or
the V_{H} region and V_{L} region of the second CAR comprises (a) the amino acid sequences set forth in SEQ ID NO:197 and SEQ ID NO:198, respectively, or (b) an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:197 and an amino acid sequence having at least at or about 90%, at or about 91%, at or about 92%, at or about 93%, at or about 94%, at or about 95%, at or about 96%, at or about 97%, at or about 98%, or at or about 99% sequence identity to SEQ ID NO:198.

245. The combination of any of claims 239-244, wherein:
the V_{H} region and V_{L} region of the second CAR comprise the amino acid sequences set forth in SEQ ID NO:189 and SEQ ID NO:190, respectively;
the V_{H} region and V_{L} region of the second CAR comprise the amino acid sequences set forth in SEQ ID NO:191 and SEQ ID NO:192;
the V_{H} region and V_{L} region of the second CAR comprise the amino acid sequences set forth in SEQ ID NO:193 and SEQ ID NO:194;
the V_{H} region and V_{L} region of the second CAR comprise the amino acid sequences set forth in SEQ ID NO:195 and SEQ ID NO:196; or
the V_{H} region and V_{L} region of the second CAR comprise the amino acid sequences set forth in SEQ ID NO:197 and SEQ ID NO:198, respectively.

246. The combination of any of claims 239-245, wherein the antigen-binding domain comprises the amino acid sequence set forth in SEQ ID NOs: 237, 238, 239, 240, or 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 227, 238, 239, 240, or 241.

247. The combination of any of claims 239-246, wherein the antigen-binding domain comprises the amino acid sequence set forth in SEQ ID NOs: 237, 238, 239, 240, or 241.

248. The combination of any of claims 239-247, wherein:
the V_{H} region of the second CAR comprises a CDR-H1, CDR-H2, and CDR-H3 comprising the amino acid sequences of SEQ ID NOS:209, 210 and 211, respectively, and the V_{L} region of the second CAR comprises a CDR-L1, CDR-L2, and CDR-L3 comprising the amino acid sequences of SEQ ID NOS:230, 231 and 232, respectively; and/or
the V_{H} region and V_{L} region of the second CAR comprise the amino acid sequences set forth in SEQ ID NO:197 and SEQ ID NO:198, respectively; and/or
the antigen-binding domain comprises the amino acid sequence set forth in SEQ ID NO: 241 or an amino acid sequence having at least at or about 90%, at or about 91%, at or about 92%, at or about 93%, at or about 94%, at or about 95%, at or about 96%, at or about 97%, at or about 98%, or at or about 99% sequence identity to the sequence set forth in SEQ ID NO:241.

249. The combination of any of claims 235-248, wherein at least one of the first chimeric antigen receptor and the second chimeric antigen receptor comprises an intracellular signaling region comprising an intracellular signaling domain of 4-1BB or a signaling portion thereof, optionally of a human 4-1BB.

250. A kit comprising the combination of any of claims 235-249 and instructions for use, optionally wherein the instructions are for administering a dose of the first and second plurality of cells, optionally in accord with the method or use or pharmaceutical composition for use of any of claims 174-216.

251. An article of manufacture comprising the combination of any of claims 235-249 or the kit of claim 250.

252. The article of manufacture of claim 251 comprising a first container comprising a dose of the plurality of first cells and a second container comprising a dose of the plurality of second cells, optionally wherein the first and second container independently is a vial or bag.

253. Use of the combination of any of claims 235-249 for the treatment of a disease or disorder, optionally wherein the disease or disorder is a cancer.

254. The use of the combination of any of claims 235-249 for the manufacture of a medicament for treatment of a disease or disorder, optionally wherein the disease or disorder is a cancer.

255. A pharmaceutical composition for treating a disease or disorder, optionally a cancer, containing the combination of any of claims 235-249 as an active ingredient.


SEQUENCE LISTING (320 sequences)

SEQ ID NO: 1 - protein, 250 aa, Artificial Sequence
  GPRC5D-200 VH/VL
EVQLVESGGAFVQPGGSLRLSCAASGFTFSSYAMTWVRQAPGKGLEWVSTISGRGRSTFYADSVKGRFTISRDNSKNTLYLQMNSLRAEDTAVYYCARYYHAGAFDLWGQGTLVTVSSGSRGGGGSGGGGSGGGGSLEMAQSVVTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVL

SEQ ID NO: 2 - protein, 250 aa, Artificial Sequence
  GPRC5D-200 - VL/VH
QSVVTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLGSRGGGGSGGGGSGGGGSLEMAEVQLVESGGAFVQPGGSLRLSCAASGFTFSSYAMTWVRQAPGKGLEWVSTISGRGRSTFYADSVKGRFTISRDNSKNTLYLQMNSLRAEDTAVYYCARYYHAGAFDLWGQGTLVTVSS

SEQ ID NO: 3 - protein, 246 aa, Artificial Sequence
  GPRC5D-201 VH/VL
QMQLVQSGAEVKKPGASVKVSCKASGYTFNRYAITWVRQAPGQGLEWMGWISAYNGNSHYAQKLQGRVTMTTDTSTGTAYMELRRLRSDDTAVYYCARMAYDSWGQGTLVTVSSGSRGGGGSGGGGSGGGGSLEMAQSVLTQPASVSGSPGQSLTISCTGTSNDVGAYKYVSWYQQYPGKAPKLILYDVFKRPSGVSNRFSGSKSDNTASLTISGLQAEDEADYYCFSLTSSNTYVFGTGTKVTVL

SEQ ID NO: 4 - protein, 246 aa, Artificial Sequence
  GPRC5D-201 VL-VH
QSVLTQPASVSGSPGQSLTISCTGTSNDVGAYKYVSWYQQYPGKAPKLILYDVFKRPSGVSNRFSGSKSDNTASLTISGLQAEDEADYYCFSLTSSNTYVFGTGTKVTVLGSRGGGGSGGGGSGGGGSLEMAQMQLVQSGAEVKKPGASVKVSCKASGYTFNRYAITWVRQAPGQGLEWMGWISAYNGNSHYAQKLQGRVTMTTDTSTGTAYMELRRLRSDDTAVYYCARMAYDSWGQGTLVTVSS

SEQ ID NO: 5 - protein, 249 aa, Artificial Sequence
  GPRC5D-202 VH/VL
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGYGKAYDQWGQGTLVTVSSGSRGGGGSGGGGSGGGGSLEMAQSVLTQPPSASGTPGQRVTISCSGSRSNVGGNYVFWYQQVPGATPKLLIYRSNQRPSGVPDRFAGSKSGSSASLAISGLRSEDEADYYCATWDDSLSGFVFGTGTKVTVL

SEQ ID NO: 6 - protein, 249 aa, Artificial Sequence
  GPRC5D-202 VL/VH
QSVLTQPPSASGTPGQRVTISCSGSRSNVGGNYVFWYQQVPGATPKLLIYRSNQRPSGVPDRFAGSKSGSSASLAISGLRSEDEADYYCATWDDSLSGFVFGTGTKVTVLGSRGGGGSGGGGSGGGGSLEMAEVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGYGKAYDQWGQGTLVTVSS

SEQ ID NO: 7 - protein, 250 aa, Artificial Sequence
  GPRC5D-203 VH/VL
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSGSRGGGGSGGGGSGGGGSLEMASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVL

SEQ ID NO: 8 - protein, 250 aa, Artificial Sequence
  GPRC5D-203 VL/VH
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 9 - protein, 250 aa, Artificial Sequence
  GPRC5D-204 VH/VL
EVQLVESGGGLVQPGGSLRLSCAASGFTFSNYAMSWVRQAPGKGLEWVSAISGSGNTYYADSVKGRFTISRDNSKNTLYLQMNSLRAEDTAVYYCARGSVRYTDIWGQGTLVTVSSGSRGGGGSGGGGSGGGGSLEMANFMLTQPHSVSESPGKTVSISCTRTSGAIAGAYVQWFQQRPGSAPTTVIYDDNKRPSGVPDRFSGSIDKSSNSASLTISGLKTEDEADYYCQSYDYDSSNVLFGGGTKLTVL

SEQ ID NO: 10 - protein, 250 aa, Artificial Sequence
  GPRC5D-204 VL/VH
NFMLTQPHSVSESPGKTVSISCTRTSGAIAGAYVQWFQQRPGSAPTTVIYDDNKRPSGVPDRFSGSIDKSSNSASLTISGLKTEDEADYYCQSYDYDSSNVLFGGGTKLTVLGSRGGGGSGGGGSGGGGSLEMAEVQLVESGGGLVQPGGSLRLSCAASGFTFSNYAMSWVRQAPGKGLEWVSAISGSGNTYYADSVKGRFTISRDNSKNTLYLQMNSLRAEDTAVYYCARGSVRYTDIWGQGTLVTVSS

SEQ ID NO: 11 - protein, 250 aa, Artificial Sequence
  GPRC5D-205 VH/VL
EVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTVSSGSRGGGGSGGGGSGGGGSLEMAQSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVL

SEQ ID NO: 12 - protein, 248 aa, Artificial Sequence
  GPRC5D-205 VL/VH
QSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTV

SEQ ID NO: 13 - protein, 253 aa, Artificial Sequence
  GPRC5D-206 VH/VL
QVQLVQSGAEVKKPGASVKVSCKASGYTFTSYYMHWVRQAPGQGLEWMGIINPSGGSTRYAQKFQGRVTMTRDTSTSTVYMELSSLRSEDTAVYYCARGSSRWGGWTGDYWGQGTLVTVSSGSRGGGGSGGGGSGGGGSLEMAQSALTQPASVSGSPGQSITISCTGTSSDVGGYNFVSWYQQHPGKAPKVMIYDVSKRPSGISNRFSGSKSGNTASLTISGLQVEDEAEYYCSSYTSTRTVIFAGGTKVTVL

SEQ ID NO: 14 - protein, 253 aa, Artificial Sequence
  GPRC5D-206 VL/VH
QSALTQPASVSGSPGQSITISCTGTSSDVGGYNFVSWYQQHPGKAPKVMIYDVSKRPSGISNRFSGSKSGNTASLTISGLQVEDEAEYYCSSYTSTRTVIFAGGTKVTVLGSRGGGGSGGGGSGGGGSLEMAQVQLVQSGAEVKKPGASVKVSCKASGYTFTSYYMHWVRQAPGQGLEWMGIINPSGGSTRYAQKFQGRVTMTRDTSTSTVYMELSSLRSEDTAVYYCARGSSRWGGWTGDYWGQGTLVTVSS

SEQ ID NO: 15 - protein, 12 aa, Artificial Sequence
  short spacer
ESKYGPPCPPCP

SEQ ID NO: 16 - protein, 119 aa, Artificial Sequence
  medium spacer (hinge-CH3 119 aa)
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 228 aa, Artificial Sequence
  long spacer (IgG4/IgG2 hinge-IgG2/IgG4 CH2- IgG4 CH3 spacer; 228 aa)
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 28 aa, Artificial Sequence
  CD28 transmembrane domain
MFWVLVVVGGVLACYSLLVTVAFIIFWV

SEQ ID NO: 19 - protein, 42 aa, Artificial Sequence
  4-1BB-derived intracellular co-signaling sequence (aa)
KRGRKKLLYIFKQPFMRPVQTTQEEDGCSCRFPEEEEGGCEL

SEQ ID NO: 20 - protein, 112 aa, Artificial Sequence
  CD3-zeta derived intracellular signaling domain (aa)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 118 aa, Artificial Sequence
  GPRC5D-200 VH
EVQLVESGGAFVQPGGSLRLSCAASGFTFSSYAMTWVRQAPGKGLEWVSTISGRGRSTFYADSVKGRFTISRDNSKNTLYLQMNSLRAEDTAVYYCARYYHAGAFDLWGQGTLVTVSS

SEQ ID NO: 22 - protein, 111 aa, Artificial Sequence
  GPRC5D-200 VL
QSVVTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVLG

SEQ ID NO: 23 - protein, 114 aa, Artificial Sequence
  GPRC5D-201 VH
QMQLVQSGAEVKKPGASVKVSCKASGYTFNRYAITWVRQAPGQGLEWMGWISAYNGNSHYAQKLQGRVTMTTDTSTGTAYMELRRLRSDDTAVYYCARMAYDSWGQGTLVTVSS

SEQ ID NO: 24 - protein, 111 aa, Artificial Sequence
  GPRC5D-201 VL
QSVLTQPASVSGSPGQSLTISCTGTSNDVGAYKYVSWYQQYPGKAPKLILYDVFKRPSGVSNRFSGSKSDNTASLTISGLQAEDEADYYCFSLTSSNTYVFGTGTKVTVLG

SEQ ID NO: 25 - protein, 117 aa, Artificial Sequence
  GPRC5D-202 VH
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGYGKAYDQWGQGTLVTVSS

SEQ ID NO: 26 - protein, 111 aa, Artificial Sequence
  GPRC5D-202 VL
QSVLTQPPSASGTPGQRVTISCSGSRSNVGGNYVFWYQQVPGATPKLLIYRSNQRPSGVPDRFAGSKSGSSASLAISGLRSEDEADYYCATWDDSLSGFVFGTGTKVTVLG

SEQ ID NO: 27 - protein, 119 aa, Artificial Sequence
  GPRC5D-203 VH
QVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 28 - protein, 110 aa, Artificial Sequence
  GPRC5D-203 VL
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLG

SEQ ID NO: 29 - protein, 116 aa, Artificial Sequence
  GPRC5D-204 VH
EVQLVESGGGLVQPGGSLRLSCAASGFTFSNYAMSWVRQAPGKGLEWVSAISGSGNTYYADSVKGRFTISRDNSKNTLYLQMNSLRAEDTAVYYCARGSVRYTDIWGQGTLVTVSS

SEQ ID NO: 30 - protein, 113 aa, Artificial Sequence
  GPRC5D-204 VL
NFMLTQPHSVSESPGKTVSISCTRTSGAIAGAYVQWFQQRPGSAPTTVIYDDNKRPSGVPDRFSGSIDKSSNSASLTISGLKTEDEADYYCQSYDYDSSNVLFGGGTKLTVLG

SEQ ID NO: 31 - protein, 116 aa, Artificial Sequence
  GPRC5D-205 VH
EVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTV

SEQ ID NO: 32 - protein, 111 aa, Artificial Sequence
  GPRC5D-205 VL
QSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLG

SEQ ID NO: 33 - protein, 121 aa, Artificial Sequence
  GPRC5D-206 VH
QVQLVQSGAEVKKPGASVKVSCKASGYTFTSYYMHWVRQAPGQGLEWMGIINPSGGSTRYAQKFQGRVTMTRDTSTSTVYMELSSLRSEDTAVYYCARGSSRWGGWTGDYWGQGTLVTVSS

SEQ ID NO: 34 - protein, 111 aa, Artificial Sequence
  GPRC5D-206 VL
QSALTQPASVSGSPGQSITISCTGTSSDVGGYNFVSWYQQHPGKAPKVMIYDVSKRPSGISNRFSGSKSGNTASLTISGLQVEDEAEYYCSSYTSTRTVIFAGGTKVTVLG

SEQ ID NO: 35 - protein, 18 aa, Artificial Sequence
  T2A peptide (aa)
EGRGSLLTCGDVEENPGP

SEQ ID NO: 36 - protein, 21 aa, Artificial Sequence
  T2A peptide (aa)
GSGEGRGSLLTCGDVEENPGP

SEQ ID NO: 37 - protein, 24 aa, Artificial Sequence
  T2A peptide (aa)
LEGGGEGRGSLLTCGDVEENPGPR

SEQ ID NO: 38 - protein, 19 aa, Artificial Sequence
  P2A peptide (aa)
ATNFSLLKQAGDVEENPGP

SEQ ID NO: 39 - protein, 22 aa, Artificial Sequence
  P2A peptide (aa)
GSGATNFSLLKQAGDVEENPGP

SEQ ID NO: 40 - protein, 20 aa, Artificial Sequence
  E2A peptide (aa)
QCTNYALLKLAGDVESNPGP

SEQ ID NO: 41 - protein, 23 aa, Artificial Sequence
  E2A peptide (aa)
GSGQCTNYALLKLAGDVESNPGP

SEQ ID NO: 42 - protein, 22 aa, Artificial Sequence
  F2A peptide (aa)
VKQTLNFDLLKLAGDVESNPGP

SEQ ID NO: 43 - protein, 25 aa, Artificial Sequence
  F2A peptide (aa)
GSGVKQTLNFDLLKLAGDVESNPGP

SEQ ID NO: 44 - DNA, 72 bp, Artificial Sequence
  T2A peptide (nt)
ctcgagggcggcggagagggcagaggaagtcttctaacatgcggtgacgtggaggagaatcccggccctagg

SEQ ID NO: 45 - DNA, 72 bp, Artificial Sequence
  T2A peptide (nt)
cttgaaggtggtggcgaaggcagaggcagcctgcttacatgcggagatgtggaagagaaccccggacctaga

SEQ ID NO: 46 - protein, 41 aa, Artificial Sequence
  CD28 co-stimulatory domain
RSKRSRLLHSDYMNMTPRRPGPTRKHYQPYAPPRDFAAYRS

SEQ ID NO: 47 - protein, 22 aa, Artificial Sequence
  Linker
GSRGGGGSGGGGSGGGGSLEMA

SEQ ID NO: 48 - DNA, 684 bp, Artificial Sequence
  long spacer (nt)
gagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 49 - protein, 345 aa, Homo sapiens
  GPRC5D protein (Uniprot Q9NZD1)
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 50 - protein, 5 aa, Artificial Sequence
  4GS linker
GGGGS

SEQ ID NO: 51 - protein, 4 aa, Artificial Sequence
  3GSlinker
GGGS

SEQ ID NO: 52 - protein, 15 aa, Artificial Sequence
  linker
GGGGSGGGGSGGGGS

SEQ ID NO: 53 - protein, 18 aa, Artificial Sequence
  linker
GSTSGSGKPGSGEGSTKG

SEQ ID NO: 54 - protein, 21 aa, Artificial Sequence
  linker
SRGGGGSGGGGSGGGGSLEMA

SEQ ID NO: 55 - DNA, 84 bp, Artificial Sequence
  CD28 transmembrane domain (nt) - not optimized
atgttttgggtgctggtcgtggtcggaggggtgctggcctgttacagcctgctggtgacagtcgctttcatcatcttctgggtg

SEQ ID NO: 56 - DNA, 84 bp, Artificial Sequence
  CD28 transmembrane domain (nt) - optimized
atgttctgggtgctcgtggtcgttggcggagtgctggcctgttacagcctgctggttaccgtggccttcatcatcttttgggtc

SEQ ID NO: 57 - DNA, 336 bp, Artificial Sequence
  CD3-zeta derived intracellular signaling domain (nt)
agagtcaagttttccaggtccgccgacgctccagcctaccagcaggggcagaaccagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 58 - DNA, 336 bp, Artificial Sequence
  CD3-zeta derived intracellular signaling domain (nt) - optimized
agagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 59 - DNA, 126 bp, Artificial Sequence
  4-1BB-derived intracellular co-signaling sequence (nt)
aagcgggggagaaagaaactgctgtatattttcaaacagccctttatgagacctgtgcagactacccaggaggaagacggatgcagctgtaggtttcccgaggaagaggaaggaggctgtgagctg

SEQ ID NO: 60 - DNA, 126 bp, Artificial Sequence
  4-1BB-derived intracellular co-signaling sequence (nt) - optimized
aagcggggcagaaagaagctgctctacatcttcaagcagcccttcatgcggcccgtgcagaccacacaagaggaagatggctgctcctgcagattccccgaggaagaagaaggcggctgcgagctg

SEQ ID NO: 61 - DNA, 544 bp, Artificial Sequence
  EF1alpha promoter with HTLV1 ehancer
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 62 - DNA, 589 bp, Artificial Sequence
  Woodchuck Hepatitis Virus (WHP) Posttranscriptional Regulatory Element (WPRE)
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - protein, 110 aa, Artificial Sequence
  GPRC5D-200 VL
QSVVTQPASVSGSPGQSITISCTGTSSDVGGYNYVSWYQQHPGKAPKLMIYDVSKRPSGVSNRFSGSKSGNTASLTISGLQAEDEADYYCSSYTSSSTLVFGGGTKLTVL

SEQ ID NO: 64 - protein, 110 aa, Artificial Sequence
  GPRC5D-201 VL
QSVLTQPASVSGSPGQSLTISCTGTSNDVGAYKYVSWYQQYPGKAPKLILYDVFKRPSGVSNRFSGSKSDNTASLTISGLQAEDEADYYCFSLTSSNTYVFGTGTKVTVL

SEQ ID NO: 65 - protein, 110 aa, Artificial Sequence
  GPRC5D-202 VL
QSVLTQPPSASGTPGQRVTISCSGSRSNVGGNYVFWYQQVPGATPKLLIYRSNQRPSGVPDRFAGSKSGSSASLAISGLRSEDEADYYCATWDDSLSGFVFGTGTKVTVL

SEQ ID NO: 66 - protein, 109 aa, Artificial Sequence
  GPRC5D-203 VL
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVL

SEQ ID NO: 67 - protein, 112 aa, Artificial Sequence
  GPRC5D-204 VL
NFMLTQPHSVSESPGKTVSISCTRTSGAIAGAYVQWFQQRPGSAPTTVIYDDNKRPSGVPDRFSGSIDKSSNSASLTISGLKTEDEADYYCQSYDYDSSNVLFGGGTKLTVL

SEQ ID NO: 68 - protein, 110 aa, Artificial Sequence
  GPRC5D-205 VL
QSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVL

SEQ ID NO: 69 - protein, 110 aa, Artificial Sequence
  GPRC5D-206 VL
QSALTQPASVSGSPGQSITISCTGTSSDVGGYNFVSWYQQHPGKAPKVMIYDVSKRPSGISNRFSGSKSGNTASLTISGLQVEDEAEYYCSSYTSTRTVIFAGGTKVTVL

SEQ ID NO: 70 - DNA, 2028 bp, Artificial Sequence
  anti-GPRC5D CAR nt
atgccgctgctgctactgctgcccctgctgtgggcaggggctctagcttcttctgagctgacccaagatcctgccgtgtctgtggctctgggccagacagtgcggattacctgtcagggcgatagcctgagaagctactacgccagctggtatcagcagaagcctggacaggctcccgtgctggtcatctacggcaagaacaacagacccagcggcatccccgatagattcagcggaagcagctctggcaataccgccagcctgacaattactggcgcccaggccgaagatgaggccgactactactgcaacagcagagacagctccggcaatcctcctgtggtttttggcggaggcaccaagctgacagtgctgggaagtagaggtggcggaggatctggcggcggaggaagcggaggcggcggatctcttgaaatggctcaggtgcagctggtggaatcaggcggtggacttgttcaccctggcggaagcctgagactgtcttgtgccgccagcggcttcaccttcagatcccacagcatgaactgggtccgacaggcccctggcaaaggccttgaatgggtgtccagcatcagcagcgacagcacctacacctactatgccgacagcgtgaagggcagattcaccatctccagagacaacgccaagaacagcctgtacctgcagatgaactccctgagagccgaggacaccgccgtgtactactgtgctagaagtggcggccagtggaagtactacgactattggggccagggcaccctggtcacagttagctctgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 71 - protein, 16 aa, Artificial Sequence
  CD33 signal sequence
MPLLLLLPLLWAGALA

SEQ ID NO: 72 - protein, 660 aa, Artificial Sequence
  anti-GPRC5D CAR
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - DNA, 684 bp, Artificial Sequence
  IgG4/IgG2 hinge- IgG2/IgG4 CH2- IgG4 CH3 spacer (nt)
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtgacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaa

SEQ ID NO: 74 - DNA, 684 bp, Artificial Sequence; identical to SEQ ID NO: 48
  optimized SSE IgG4/IgG2 hinge- IgG2/IgG4 CH2- IgG4 CH3 spacer (nt)

SEQ ID NO: 75 - protein, 7 aa, Artificial Sequence
  CDRH1
GFTFSSY

SEQ ID NO: 76 - protein, 6 aa, Artificial Sequence
  CDRH2
SGRGRS

SEQ ID NO: 77 - protein, 9 aa, Artificial Sequence
  CDRH3
YYHAGAFDL

SEQ ID NO: 78 - protein, 10 aa, Artificial Sequence
  CDRH1
GFTFSSYAMT

SEQ ID NO: 79 - protein, 10 aa, Artificial Sequence
  CDRH2
TISGRGRSTF

SEQ ID NO: 80 - protein, 5 aa, Artificial Sequence
  CDRH1
SYAMT

SEQ ID NO: 81 - protein, 17 aa, Artificial Sequence
  CDRH2
TISGRGRSTFYADSVKG

SEQ ID NO: 82 - protein, 8 aa, Artificial Sequence
  CDRH1
GFTFSSYA

SEQ ID NO: 83 - protein, 8 aa, Artificial Sequence
  CDRH2
ISGRGRST

SEQ ID NO: 84 - protein, 11 aa, Artificial Sequence
  CDRH3
ARYYHAGAFDL

SEQ ID NO: 85 - protein, 14 aa, Artificial Sequence
  CDRL1
TGTSSDVGGYNYVS

SEQ ID NO: 86 - protein, 7 aa, Artificial Sequence
  CDRL2
DVSKRPS

SEQ ID NO: 87 - protein, 10 aa, Artificial Sequence
  CDRL3
SSYTSSSTLV

SEQ ID NO: 88 - protein, 9 aa, Artificial Sequence
  CDRL1
SSDVGGYNY

SEQ ID NO: 89 - protein, 3 aa, Artificial Sequence
  CDRL2
DVS

SEQ ID NO: 90 - protein, 7 aa, Artificial Sequence
  CDRH1
GYTFNRY

SEQ ID NO: 91 - protein, 6 aa, Artificial Sequence
  CDRH2
SAYNGN

SEQ ID NO: 92 - protein, 5 aa, Artificial Sequence
  CDRH3
MAYDS

SEQ ID NO: 93 - protein, 10 aa, Artificial Sequence
  CDRH1
GYTFNRYAIT

SEQ ID NO: 94 - protein, 10 aa, Artificial Sequence
  CDRH2
WISAYNGNSH

SEQ ID NO: 95 - protein, 5 aa, Artificial Sequence
  CDRH1
RYAIT

SEQ ID NO: 96 - protein, 17 aa, Artificial Sequence
  CDRH2
WISAYNGNSHYAQKLQG

SEQ ID NO: 97 - protein, 8 aa, Artificial Sequence
  CDRH1
GYTFNRYA

SEQ ID NO: 98 - protein, 8 aa, Artificial Sequence
  CDRH2
ISAYNGNS

SEQ ID NO: 99 - protein, 7 aa, Artificial Sequence
  CDRH3
ARMAYDS

SEQ ID NO: 100 - protein, 14 aa, Artificial Sequence
  CDRL1
TGTSNDVGAYKYVS

SEQ ID NO: 101 - protein, 7 aa, Artificial Sequence
  CDRL2
DVFKRPS

SEQ ID NO: 102 - protein, 10 aa, Artificial Sequence
  CDRL3
FSLTSSNTYV

SEQ ID NO: 103 - protein, 9 aa, Artificial Sequence
  CDRL1
SNDVGAYKY

SEQ ID NO: 104 - protein, 3 aa, Artificial Sequence
  CDRL2
DVF

SEQ ID NO: 105 - protein, 7 aa, Artificial Sequence
  CDRH1
GFTFSDY

SEQ ID NO: 106 - protein, 6 aa, Artificial Sequence
  CDRH2
SSSGST

SEQ ID NO: 107 - protein, 8 aa, Artificial Sequence
  CDRH3
GYGKAYDQ

SEQ ID NO: 108 - protein, 10 aa, Artificial Sequence
  CDRH1
GFTFSDYYMS

SEQ ID NO: 109 - protein, 10 aa, Artificial Sequence
  CDRH2
YISSSGSTIY

SEQ ID NO: 110 - protein, 5 aa, Artificial Sequence
  CDRH1
DYYMS

SEQ ID NO: 111 - protein, 17 aa, Artificial Sequence
  CDRH2
YISSSGSTIYYADSVKG

SEQ ID NO: 112 - protein, 8 aa, Artificial Sequence
  CDRH1
GFTFSDYY

SEQ ID NO: 113 - protein, 8 aa, Artificial Sequence
  CDRH2
ISSSGSTI

SEQ ID NO: 114 - protein, 10 aa, Artificial Sequence
  CDRH3
ARGYGKAYDQ

SEQ ID NO: 115 - protein, 13 aa, Artificial Sequence
  CDRL1
SGSRSNVGGNYVF

SEQ ID NO: 116 - protein, 7 aa, Artificial Sequence
  CDRL2
RSNQRPS

SEQ ID NO: 117 - protein, 11 aa, Artificial Sequence
  CDRL3
ATWDDSLSGFV

SEQ ID NO: 118 - protein, 8 aa, Artificial Sequence
  CDRL1
RSNVGGNY

SEQ ID NO: 119 - protein, 3 aa, Artificial Sequence
  CDRL2
RSN

SEQ ID NO: 120 - protein, 7 aa, Artificial Sequence
  CDRH1
GFTFRSH

SEQ ID NO: 121 - protein, 6 aa, Artificial Sequence
  CDRH2
SSDSTY

SEQ ID NO: 122 - protein, 10 aa, Artificial Sequence
  CDRH3
SGGQWKYYDY

SEQ ID NO: 123 - protein, 10 aa, Artificial Sequence
  CDRH1
GFTFRSHSMN

SEQ ID NO: 124 - protein, 10 aa, Artificial Sequence
  CDRH2
SISSDSTYTY

SEQ ID NO: 125 - protein, 5 aa, Artificial Sequence
  CDRH1
SHSMN

SEQ ID NO: 126 - protein, 17 aa, Artificial Sequence
  CDRH2
SISSDSTYTYYADSVKG

SEQ ID NO: 127 - protein, 8 aa, Artificial Sequence
  CDRH1
GFTFRSHS

SEQ ID NO: 128 - protein, 8 aa, Artificial Sequence
  CDRH2
ISSDSTYT

SEQ ID NO: 129 - protein, 12 aa, Artificial Sequence
  CDRH3
ARSGGQWKYYDY

SEQ ID NO: 130 - protein, 11 aa, Artificial Sequence
  CDRL1
QGDSLRSYYAS

SEQ ID NO: 131 - protein, 7 aa, Artificial Sequence
  CDRL2
GKNNRPS

SEQ ID NO: 132 - protein, 12 aa, Artificial Sequence
  CDRL3
NSRDSSGNPPVV

SEQ ID NO: 133 - protein, 6 aa, Artificial Sequence
  CDRL1
SLRSYY

SEQ ID NO: 134 - protein, 3 aa, Artificial Sequence
  CDRL2
GKN

SEQ ID NO: 135 - protein, 7 aa, Artificial Sequence
  CDRH1
GFTFSNY

SEQ ID NO: 136 - protein, 5 aa, Artificial Sequence
  CDRH2
SGSGN

SEQ ID NO: 137 - protein, 8 aa, Artificial Sequence
  CDRH3
GSVRYTDI

SEQ ID NO: 138 - protein, 10 aa, Artificial Sequence
  CDRH1
GFTFSNYAMS

SEQ ID NO: 139 - protein, 9 aa, Artificial Sequence
  CDRH2
AISGSGNTY

SEQ ID NO: 140 - protein, 5 aa, Artificial Sequence
  CDRH1
NYAMS

SEQ ID NO: 141 - protein, 16 aa, Artificial Sequence
  CDRH2
AISGSGNTYYADSVKG

SEQ ID NO: 142 - protein, 8 aa, Artificial Sequence
  CDRH1
GFTFSNYA

SEQ ID NO: 143 - protein, 7 aa, Artificial Sequence
  CDRH2
ISGSGNT

SEQ ID NO: 144 - protein, 10 aa, Artificial Sequence
  CDRH3
ARGSVRYTDI

SEQ ID NO: 145 - protein, 13 aa, Artificial Sequence
  CDRL1
TRTSGAIAGAYVQ

SEQ ID NO: 146 - protein, 7 aa, Artificial Sequence
  CDRL2
DDNKRPS

SEQ ID NO: 147 - protein, 11 aa, Artificial Sequence
  CDRL3
QSYDYDSSNVL

SEQ ID NO: 148 - protein, 8 aa, Artificial Sequence
  CDRL1
SGAIAGAY

SEQ ID NO: 149 - protein, 3 aa, Artificial Sequence
  CDRL2
DDN

SEQ ID NO: 150 - protein, 6 aa, Artificial Sequence
  CDRH2
NGRGSS

SEQ ID NO: 151 - protein, 9 aa, Artificial Sequence
  CDRH3
YISRGLGDS

SEQ ID NO: 152 - protein, 10 aa, Artificial Sequence
  CDRH1
GFTFSNYAMN

SEQ ID NO: 153 - protein, 10 aa, Artificial Sequence
  CDRH2
TINGRGSSTI

SEQ ID NO: 154 - protein, 17 aa, Artificial Sequence
  CDRH2
TINGRGSSTIYADSVKG

SEQ ID NO: 155 - protein, 8 aa, Artificial Sequence
  CDRH2
INGRGSST

SEQ ID NO: 156 - protein, 11 aa, Artificial Sequence
  CDRH3
ARYISRGLGDS

SEQ ID NO: 157 - protein, 13 aa, Artificial Sequence
  CDRL1
SGGNSNIERNYVS

SEQ ID NO: 158 - protein, 7 aa, Artificial Sequence
  CDRL2
DNDRRPS

SEQ ID NO: 159 - protein, 11 aa, Artificial Sequence
  CDRL3
GTWDSSLRGWV

SEQ ID NO: 160 - protein, 8 aa, Artificial Sequence
  CDRL1
NSNIERNY

SEQ ID NO: 161 - protein, 3 aa, Artificial Sequence
  CDRL2
DND

SEQ ID NO: 162 - protein, 7 aa, Artificial Sequence
  CDRH1
GYTFTSY

SEQ ID NO: 163 - protein, 6 aa, Artificial Sequence
  CDRH2
NPSGGS

SEQ ID NO: 164 - protein, 12 aa, Artificial Sequence
  CDRH3
GSSRWGGWTGDY

SEQ ID NO: 165 - protein, 10 aa, Artificial Sequence
  CDRH1
GYTFTSYYMH

SEQ ID NO: 166 - protein, 10 aa, Artificial Sequence
  CDRH2
IINPSGGSTR

SEQ ID NO: 167 - protein, 5 aa, Artificial Sequence
  CDRH1
SYYMH

SEQ ID NO: 168 - protein, 17 aa, Artificial Sequence
  CDRH2
IINPSGGSTRYAQKFQG

SEQ ID NO: 169 - protein, 8 aa, Artificial Sequence
  CDRH1
GYTFTSYY

SEQ ID NO: 170 - protein, 8 aa, Artificial Sequence
  CDRH2
INPSGGST

SEQ ID NO: 171 - protein, 13 aa, Artificial Sequence
  CDRH3
ARGSSRWGGWTGD

SEQ ID NO: 172 - protein, 14 aa, Artificial Sequence
  CDRL1
TGTSSDVGGYNFVS

SEQ ID NO: 173 - protein, 10 aa, Artificial Sequence
  CDRL3
SSYTSTRTVI

SEQ ID NO: 174 - protein, 9 aa, Artificial Sequence
  CDRL1
SSDVGGYNF

SEQ ID NO: 175 - protein, 20 aa, Artificial Sequence
  CDRL3
SSYTSTRTVIFAGGTKVTVL

SEQ ID NO: 176 - DNA, 15 bp, Artificial Sequence
  splice donor site
aatctaagtacggac

SEQ ID NO: 177 - DNA, 15 bp, Artificial Sequence
  splice donor site
tcaactggtacgtgg

SEQ ID NO: 178 - DNA, 15 bp, Artificial Sequence
  splice donor site
acaattagtaaggca

SEQ ID NO: 179 - DNA, 15 bp, Artificial Sequence
  splice donor site
accacaggtgtatac

SEQ ID NO: 180 - DNA, 41 bp, Artificial Sequence
  splice acceptor site
aagtttctttctgtattccaggctgaccgtggataaatctc

SEQ ID NO: 181 - DNA, 41 bp, Artificial Sequence
  splice acceptor site
gggcaacgtgttctcttgcagtgtcatgcacgaagccctgc

SEQ ID NO: 182 - DNA, 15 bp, Artificial Sequence
  modified splice donor site
agtctaaatacggac

SEQ ID NO: 183 - DNA, 15 bp, Artificial Sequence
  modified splice donor site
tcaactggtatgtgg

SEQ ID NO: 184 - DNA, 15 bp, Artificial Sequence
  modified splice donor site
accatctccaaggcc

SEQ ID NO: 185 - DNA, 15 bp, Artificial Sequence
  modified splice donor site
gccccaggtttacac

SEQ ID NO: 186 - DNA, 41 bp, Artificial Sequence
  modified splice acceptor site
cagtttcttcctgtatagtagactcaccgtggataaatcaa

SEQ ID NO: 187 - DNA, 41 bp, Artificial Sequence
  modified splice acceptor site
gggcaacgtgttcagctgcagcgtgatgcacgaggccctgc

SEQ ID NO: 188 - DNA, 41 bp, Artificial Sequence
  modified splice acceptor site
cgccttgtcctccttgtcccgctcctcctgttgccggacct

SEQ ID NO: 189 - protein, 118 aa, Artificial Sequence
  BCMA-23 VH
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSS

SEQ ID NO: 190 - protein, 110 aa, Artificial Sequence
  BCMA-23 VL
QSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 191 - protein, 118 aa, Artificial Sequence
  BCMA-25 VH
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSS

SEQ ID NO: 192 - protein, 107 aa, Artificial Sequence
  BCMA-25 VL
DIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 193 - protein, 118 aa, Artificial Sequence
  BCMA-26 VH
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSS

SEQ ID NO: 194 - protein, 108 aa, Artificial Sequence
  BCMA-26 VL
SYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 195 - protein, 116 aa, Artificial Sequence
  BCMA-52 VH
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 196 - protein, 111 aa, Artificial Sequence
  BCMA-52 VL
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 197 - protein, 118 aa, Artificial Sequence
  BCMA-55 VH
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 198 - protein, 105 aa, Artificial Sequence
  BCMA-55 VL
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 199 - protein, 5 aa, Artificial Sequence
  CDRH1
DYYMS

SEQ ID NO: 200 - protein, 17 aa, Artificial Sequence
  CDRH2
YISSSGSTIYYADSVKG

SEQ ID NO: 201 - protein, 9 aa, Artificial Sequence
  CDRH3
VDGDYTEDY

SEQ ID NO: 202 - protein, 5 aa, Artificial Sequence
  CDRH1
DYAMS

SEQ ID NO: 203 - protein, 19 aa, Artificial Sequence
  CDRH2
FIRSKAYGGTTEYAASVKG

SEQ ID NO: 204 - protein, 7 aa, Artificial Sequence
  CDRH3
WSAPTDY

SEQ ID NO: 205 - protein, 9 aa, Artificial Sequence
  CDRH3
VDGPPSFDI

SEQ ID NO: 206 - protein, 5 aa, Artificial Sequence
  CDRH1
SYWIG

SEQ ID NO: 207 - protein, 17 aa, Artificial Sequence
  CDRH2
IIYPGDSDTRYSPSFQG

SEQ ID NO: 208 - protein, 7 aa, Artificial Sequence
  CDRH3
YSGSFDN

SEQ ID NO: 209 - protein, 5 aa, Artificial Sequence
  CDRH1
DYYVY

SEQ ID NO: 210 - protein, 17 aa, Artificial Sequence
  CDRH2
WINPNSGGTNYAQKFQG

SEQ ID NO: 211 - protein, 9 aa, Artificial Sequence
  CDRH3
SQRDGYMDY

SEQ ID NO: 212 - protein, 8 aa, Artificial Sequence
  CDRH1
GYSFTSYW

SEQ ID NO: 213 - protein, 8 aa, Artificial Sequence
  CDRH2
IYPGDSDT

SEQ ID NO: 214 - protein, 9 aa, Artificial Sequence
  CDRH3
ARYSGSFDN

SEQ ID NO: 215 - protein, 8 aa, Artificial Sequence
  CDRH1
GYTFIDYY

SEQ ID NO: 216 - protein, 8 aa, Artificial Sequence
  CDRH2
INPNSGGT

SEQ ID NO: 217 - protein, 11 aa, Artificial Sequence
  CDRH3
ARSQRDGYMDY

SEQ ID NO: 218 - protein, 14 aa, Artificial Sequence
  CDRL1
TGSSSDVGKYNLVS

SEQ ID NO: 219 - protein, 7 aa, Artificial Sequence
  CDRL2
DVNKRPS

SEQ ID NO: 220 - protein, 10 aa, Artificial Sequence
  CDRL3
SSYGGSRSYV

SEQ ID NO: 221 - protein, 11 aa, Artificial Sequence
  CDRL1
RASQGISNYLA

SEQ ID NO: 222 - protein, 7 aa, Artificial Sequence
  CDRL2
SASTLQS

SEQ ID NO: 223 - protein, 9 aa, Artificial Sequence
  CDRL3
QQSYTSRQT

SEQ ID NO: 224 - protein, 11 aa, Artificial Sequence
  CDRL1
GANNIGSKSVH

SEQ ID NO: 225 - protein, 7 aa, Artificial Sequence
  CDRL2
DDDDRPS

SEQ ID NO: 226 - protein, 11 aa, Artificial Sequence
  CDRL3
HLWDRSRDHYV

SEQ ID NO: 227 - protein, 13 aa, Artificial Sequence
  CDRL1
SGTSSNIGSHSVN

SEQ ID NO: 228 - protein, 7 aa, Artificial Sequence
  CDRL2
TNNQRPS

SEQ ID NO: 229 - protein, 11 aa, Artificial Sequence
  CDRL3
AAWDGSLNGLV

SEQ ID NO: 230 - protein, 8 aa, Artificial Sequence
  CDRL1
TGTSSDVG

SEQ ID NO: 231 - protein, 7 aa, Artificial Sequence
  CDRL2
EDSKRPS

SEQ ID NO: 232 - protein, 10 aa, Artificial Sequence
  CDRL3
SSNTRSSTLV

SEQ ID NO: 233 - protein, 8 aa, Artificial Sequence
  CDRL1
SSNIGSHS

SEQ ID NO: 234 - protein, 3 aa, Artificial Sequence
  CDRL2
TNN

SEQ ID NO: 235 - protein, 9 aa, Artificial Sequence
  CDRL1
ISCTGTSSD

SEQ ID NO: 236 - protein, 3 aa, Artificial Sequence
  CDRL2
EDS

SEQ ID NO: 237 - protein, 243 aa, Artificial Sequence
  BCMA-23 scFv
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 238 - protein, 240 aa, Artificial Sequence
  BCMA-25 scFv
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 239 - protein, 241 aa, Artificial Sequence
  BCMA-26 scFv
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 240 - protein, 248 aa, Artificial Sequence
  BCMA-52 scFv
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 241 - protein, 244 aa, Artificial Sequence
  BCMA-55 scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 242 - DNA, 1959 bp, Artificial Sequence
  anti-BMCA CAR
gaggtgcagctggtggagtccggaggaggcctggtgaagccaggaggctccctgaggctgtcttgcgcagccagcggcttcacctttagcgactactatatgtcctggatcagacaggcacctggcaagggcctggagtgggtgagctacatcagctcctctggctccacaatctactatgccgactctgtgaagggccggtttaccatcagcagagataacgccaagaattccctgtatctgcagatgaacagcctgagggccgaggacacagccgtgtactattgcgccaaggtggacggcgattacaccgaggattattggggccagggcacactggtgaccgtgagctccggcggcggcggctctggaggaggaggcagcggcggaggaggctcccagtctgccctgacacagccagccagcgtgtccggctctcccggacagtccatcacaatctcttgtaccggctctagctccgacgtgggcaagtacaacctggtgtcctggtatcagcagccccctggcaaggcccctaagctgatcatctacgatgtgaacaagaggccatctggcgtgagcaatcgcttcagcggctccaagtctggcaataccgccacactgaccatcagcggcctgcagggcgacgatgaggcagattactattgttctagctacggcggcagcagatcctacgtgttcggcacaggcaccaaggtgaccgtgctggaatctaagtacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 243 - DNA, 1950 bp, Artificial Sequence
  anti-BMCA CAR
gaggtgcagctggtgcagagcggaggaggcctggtgcagcctggcaggtccctgcgcctgtcttgcaccgccagcggcttcacatttggcgactatgccatgtcctggttcaagcaggcaccaggcaagggcctggagtgggtgggctttatccgctctaaggcctacggcggcaccacagagtatgccgccagcgtgaagggccggttcaccatcagccgggacgactctaagagcatcgcctacctgcagatgaactctctgaagaccgaggacacagccgtgtactattgcgcagcatggagcgccccaaccgattattggggccagggcaccctggtgacagtgagctccggcggcggcggctctggaggaggaggaagcggaggaggaggatccgacatccagatgacacagtcccctgcctttctgtccgcctctgtgggcgatagggtgaccgtgacatgtcgcgcctcccagggcatctctaactacctggcctggtatcagcagaagcccggcaatgcccctcggctgctgatctacagcgcctccaccctgcagagcggagtgccctcccggttcagaggaaccggctatggcacagagttttctctgaccatcgacagcctgcagccagaggatttcgccacatactattgtcagcagtcttacaccagccggcagacatttggccccggcacaagactggatatcaaggagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 244 - DNA, 1953 bp, Artificial Sequence
  anti-BMCA CAR
gaggtgcagctggtggagtccggaggaggcctggtgaagccaggaggctctctgaggctgagctgcgcagcctccggcttcaccttttctgactactatatgagctggatcaggcaggcaccaggcaagggcctggagtgggtgtcttacatcagctcctctggcagcacaatctactatgccgactccgtgaagggcaggttcaccatctctcgcgataacgccaagaatagcctgtatctgcagatgaactccctgcgggccgaggatacagccgtgtactattgcgccaaggtggacggccccccttcctttgatatctggggccagggcacaatggtgaccgtgagctccggaggaggaggatccggcggaggaggctctggcggcggcggctctagctatgtgctgacccagccaccatccgtgtctgtggcacctggacagacagcaaggatcacctgtggagcaaacaatatcggcagcaagtccgtgcactggtaccagcagaagcctggccaggccccaatgctggtggtgtatgacgatgacgatcggcccagcggcatccctgagagattttctggcagcaactccggcaataccgccacactgaccatctctggagtggaggcaggcgacgaggcagattacttctgtcacctgtgggaccggagcagagatcactacgtgttcggcacaggcaccaagctgaccgtgctggaatctaagtacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 245 - DNA, 1974 bp, Artificial Sequence
  anti-BMCA CAR
agctatgagctgacacagcctccaagcgcctctggcacacctggacagcgagtgacaatgagctgtagcggcaccagcagcaacatcggcagccacagcgtgaactggtatcagcagctgcctggcacagcccctaaactgctgatctacaccaacaaccagcggcctagcggcgtgcccgatagattttctggcagcaagagcggcacaagcgccagcctggctatttctggactgcagagcgaggacgaggccgactattattgtgccgcctgggacggctctctgaacggccttgtttttggcggaggcaccaagctgacagtgctgggatctagaggtggcggaggatctggcggcggaggaagcggaggcggcggatctcttgaaatggctgaagtgcagctggtgcagtctggcgccgaagtgaagaagcctggcgagagcctgaagatcagctgcaaaggcagcggctacagcttcaccagctactggatcggctgggtccgacagatgcctggcaaaggccttgagtggatgggcatcatctaccccggcgacagcgacaccagatacagccctagctttcagggccacgtgaccatcagcgccgacaagtctatcagcaccgcctacctgcagtggtccagcctgaaggcctctgacaccgccatgtactactgcgccagatactctggcagcttcgacaattggggccagggcacactggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 246 - DNA, 1962 bp, Artificial Sequence
  anti-BMCA CAR
cagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 247 - protein, 653 aa, Artificial Sequence
  anti-BMCA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 248 - protein, 650 aa, Artificial Sequence
  anti-BMCA CAR
EVQLVQSGGGLVQPGRSLRLSCTASGFTFGDYAMSWFKQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 249 - protein, 651 aa, Artificial Sequence
  anti-BMCA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 250 - protein, 658 aa, Artificial Sequence
  anti-BMCA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 251 - protein, 654 aa, Artificial Sequence
  anti-BMCA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 252 - protein, 653 aa, Artificial Sequence
  anti-BMCA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 253 - DNA, 22 bp, Artificial Sequence
  GPRC5D CAR forward primer
actgcatcgagtccactggaga

SEQ ID NO: 254 - DNA, 22 bp, Artificial Sequence
  GPRC5D CAR reverse primer
ggatctttcgcatgaggaagag

SEQ ID NO: 255 - DNA, 19 bp, Artificial Sequence
  housekeeping gene B-actin forward primer
agcatcccccaaagttcac

SEQ ID NO: 256 - DNA, 21 bp, Artificial Sequence
  housekeeping gene B-actin reverse primer
aagggacttcctgtaacaacg

SEQ ID NO: 257 - DNA, 750 bp, Artificial Sequence
  GPRC5D-200 VH/VL (nt)
gaagtgcagctggttgaaagcggcggagcctttgttcagcctggcggatctctgagactgagctgtgccgccagcggcttcacctttagcagctacgccatgacctgggtccgacaggctcctggcaaaggccttgaatgggtgtccaccatcagcggcagaggcagaagcaccttctacgccgatagcgtgaagggcagattcaccatctccagagacaacagcaagaacaccctgtacctgcagatgaacagcctgagagccgaggacaccgccgtgtactactgcgccagatattatcacgctggcgccttcgatctgtggggccagggaacactggtcacagtgtctagtggaagcagaggcggcggaggatctggcggaggcggtagcggtggtggtggatctcttgaaatggcccagagcgtggtcacacagcctgcctctgtttctggctctcctggccagagcatcacaatcagctgtaccggcaccagctctgacgtcggcggctacaattacgtgtcctggtatcagcagcaccccggcaaggcccctaagctgatgatctacgacgtgtccaagaggcccagcggcgtgtccaatagattcagcggctccaagagcggcaacaccgccagccttacaatcagcggactgcaggccgaggacgaggccgattactactgcagcagctacaccagcagcagcacactggtttttggaggcggcaccaagctgaccgtgctt

SEQ ID NO: 258 - DNA, 750 bp, Artificial Sequence
  GPRC5D-200 - VL/VH (nt)
cagtctgtggttacacagcctgccagcgtgtccggatctcctggccagagcatcaccatcagctgtaccggcaccagctctgatgtcggcggctacaattacgtgtcctggtatcagcagcaccccggcaaggcccctaagctgatgatctacgacgtgtccaagaggcccagcggcgtgtccaatagattcagcggcagcaagagcggcaacaccgccagcctgacaattagcggactgcaggccgaggacgaggccgattactactgtagcagctacaccagcagcagcacactggtgtttggcggaggcaccaagctgacagtgcttggaagtagaggcggcggaggaagcggaggcggaggatctggtggtggtggatctctggaaatggccgaggtgcagctggtggaatctggcggagcttttgttcagcctggcggcagcctgagactgtcttgtgctgccagcggcttcaccttcagcagctacgccatgacctgggtccgacaggctcctggcaaaggccttgaatgggtgtccaccatctccggcagaggcagaagcaccttctacgccgatagcgtgaagggcagattcacaatcagccgggacaacagcaagaacaccctgtacctgcagatgaactccctgagagccgaggacaccgccgtgtactactgcgccagatattatcacgctggcgccttcgatctgtggggccagggaacactggtcaccgttagttct

SEQ ID NO: 259 - DNA, 738 bp, Artificial Sequence
  GPRC5D-201 VH/VL (nt)
caaatgcagctggttcagtctggcgccgaagtgaagaaacctggcgcctctgtgaaggtgtcctgcaaggccagcggctacaccttcaatagatacgccatcacctgggtccgacaggcccctggacaaggacttgaatggatgggctggatcagcgcctacaacggcaatagccactacgcccagaaactgcagggcagagtgaccatgaccaccgatacctctaccggcaccgcctacatggaactgcggagactgagaagcgacgacaccgccgtgtactactgtgccagaatggcctacgatagctggggccagggaaccctggttacagtgtctagtggaagcagaggcggcggaggatctggcggaggtggtagtggcggaggcggatctcttgaaatggcccagtctgtgctgacccagcctgcctctgtttctggctctcctggccaaagcctgaccatctcttgtaccggcacaagcaacgacgtgggagcctacaaatacgtgtcctggtatcagcagtaccccggcaaggcccctaagctgatcctgtacgacgtgttcaagaggcccagcggcgtgtccaatagattcagcggcagcaagtccgacaacaccgccagcctgacaatcagcggactgcaggccgaagatgaggccgactactactgcttcagcctgacctccagcaacacctacgtgttcggcaccggcaccaaagtgacagtgctt

SEQ ID NO: 260 - DNA, 738 bp, Artificial Sequence
  GPRC5D-201 VL-VH (nt)
caatctgtgctgacacagcctgccagcgtgtccggatctcctggacagagcctgaccatcagctgtaccggcaccagcaatgacgtgggcgcctacaaatacgtgtcctggtatcagcagtaccccggcaaggcccctaagctgatcctgtacgacgtgttcaagaggcccagcggcgtgtccaatagattcagcggcagcaagagcgacaacaccgccagcctgacaattagcggactgcaggccgaggacgaggccgactactactgttttagcctgacctccagcaacacctacgtgttcggcaccggcacaaaagtgacagtgctgggaagtagaggcggcggaggatctggcggaggtggaagtggcggaggcggatctcttgaaatggcccagatgcagctggtgcagtctggcgccgaagtgaaaaaacctggcgcctccgtgaaggtgtcctgcaaggcttctggctacaccttcaacagatacgccatcacctgggtccgacaggcccctggacaaggacttgaatggatgggctggatctccgcctacaacggcaatagccactacgcccagaaactgcagggcagagtgaccatgaccaccgatacctctaccggcacagcctacatggaactgcggagactgagatccgacgacaccgccgtgtactactgtgccagaatggcctacgatagctggggccagggaaccctggttaccgtttcttct

SEQ ID NO: 261 - DNA, 747 bp, Artificial Sequence
  GPRC5D-202 VH/VL (nt)
gaagtgcagctggttgaatctggcggcggactggttaagcctggcggatctctgagactgagctgtgccgccagcggcttcaccttcagcgactactacatgagctggatcagacaggcccctggcaaaggcctggaatgggtgtcctacatcagcagctctggcagcaccatctactacgccgacagcgtgaagggcagattcaccatcagccgggacaacgccaagaacagcctgtacctgcagatgaactccctgagagccgaggacaccgccgtgtactattgtgccagaggctacggcaaggcctacgatcaatggggccagggcacactggtcacagtgtctagtggaagtagaggcggcggaggatctggcggaggtggaagtggcggaggcggttctcttgaaatggcccagtctgtgctgacccagcctccttctgcttctggcacacctggccagagagtgaccatcagctgtagcggcagcagaagcaacgtcggcggcaactacgtgttctggtatcagcaggtcccaggcgccacacctaaactgctgatctacagaagcaatcagcggcccagcggcgtgcccgatagatttgccggatctaagtctggcagctccgccagcctggccatttctggactgagatctgaggacgaggccgattactactgcgccacctgggatgatagcctgagcggctttgtgtttggcaccggcaccaaagtgaccgtgctt

SEQ ID NO: 262 - DNA, 747 bp, Artificial Sequence
  GPRC5D-202 VL/VH (nt)
caatctgtgctgacacagcctcctagcgcctctggaacacctggccagagagtgaccatcagctgtagcggcagcagaagcaacgtcggcggcaactacgtgttctggtatcagcaggtcccaggcgccacacctaagctgctgatctacagaagcaatcagcggcctagcggcgtgcccgatagatttgccggaagcaagagcggcagctctgccagccttgccatctctggactgagaagcgaggacgaggccgactactactgtgccacctgggatgatagcctgagcggcttcgtgtttggcaccggcaccaaagtgacagtgctgggaagtagaggcggcggaggatctggcggaggtggaagtggcggaggcggatctcttgaaatggccgaggtgcagctggtggaatctggtggcggacttgtgaagcctggcggctctctgagactgtcttgtgccgccagcggcttcaccttcagcgattactacatgagctggatcagacaggcccctggcaaaggcctggaatgggtgtcctacatcagctccagcggctctaccatctactacgccgacagcgtgaagggcagattcaccatcagccgggacaacgccaagaacagcctgtacctgcagatgaactccctgagagccgaggacaccgccgtgtactattgtgccagaggctacggcaaggcctacgatcaatggggccagggcacactggtcaccgttagttct

SEQ ID NO: 263 - DNA, 750 bp, Artificial Sequence
  GPRC5D-203 VH/VL (nt)
caagttcagctggtggaatctggcggcggactggttcatcctggcggatctctgagactgagctgtgccgccagcggcttcacctttagaagccacagcatgaactgggtccgacaggcccctggcaaaggccttgaatgggtgtccagcatcagcagcgacagcacctacacctactacgccgacagcgtgaagggcagattcaccatctccagagacaacgccaagaacagcctgtacctgcagatgaactccctgagagccgaggacaccgccgtgtactactgtgctagatctggcggacagtggaagtactacgactactggggccagggcaccctggtcacagttagctctggaagtagaggcggcggaggaagcggaggcggaggttctggtggcggaggatctctggaaatggccagcagcgaactgacacaggaccctgcagtgtctgtggccctgggccagacagtgcggattacttgtcagggcgacagcctgcggagctactatgcctcttggtatcagcagaagcccggccaggctcctgtgctggttatctacggcaagaacaacagacccagcggcatccccgatagattcagcggaagcagctctggcaataccgccagcctgacaattactggcgcccaggccgaagatgaggccgactactactgcaacagcagagacagctccggcaatcctcctgtggtttttggcggaggcaccaagctgacagtgctc

SEQ ID NO: 264 - DNA, 750 bp, Artificial Sequence
  GPRC5D-203 VL/VH (nt)
tcttctgagctgacccaagatcctgccgtgtctgtggctctgggccagacagtgcggattacctgtcagggcgatagcctgagaagctactacgccagctggtatcagcagaagcctggacaggctcccgtgctggtcatctacggcaagaacaacagacccagcggcatccccgatagattcagcggaagcagctctggcaataccgccagcctgacaattactggcgcccaggccgaagatgaggccgactactactgcaacagcagagacagctccggcaatcctcctgtggtttttggcggaggcaccaagctgacagtgctgggaagtagaggtggcggaggatctggcggcggaggaagcggaggcggcggatctcttgaaatggctcaggtgcagctggtggaatcaggcggtggacttgttcaccctggcggaagcctgagactgtcttgtgccgccagcggcttcaccttcagatcccacagcatgaactgggtccgacaggcccctggcaaaggccttgaatgggtgtccagcatcagcagcgacagcacctacacctactatgccgacagcgtgaagggcagattcaccatctccagagacaacgccaagaacagcctgtacctgcagatgaactccctgagagccgaggacaccgccgtgtactactgtgctagaagtggcggccagtggaagtactacgactattggggccagggcaccctggtcacagttagctct

SEQ ID NO: 265 - DNA, 750 bp, Artificial Sequence
  GPRC5D-204 VH/VL (nt)
gaagtgcagctggttgaatctggcggcggactggttcaacctggcggatctctgagactgagctgtgccgccagcggcttcaccttcagcaattacgccatgagctgggtccgacaggcccctggaaaaggccttgaatgggtgtccgccatcagcggcagcggcaatacctactacgccgactctgtgaagggcagattcaccatcagccgggacaacagcaagaacaccctgtacctgcagatgaacagcctgagagccgaggacaccgccgtgtactattgtgccagaggcagcgtgcggtacaccgatatttggggccagggcacactggtcacagtgtctagtggaagtagaggcggcggaggatctggcggaggtggaagtggcggaggcggttctctggaaatggccaacttcatgctgacccagcctcacagcgtgtccgagtctccaggcaagaccgtgtccatcagctgcaccagaacaagcggagccattgccggcgcttacgtgcagtggttccagcagaggcctggaagcgctcctaccaccgtgatctacgacgacaacaagaggcctagcggcgtgcccgatagattcagcggctccatcgacaagagcagcaacagcgccagcctgacaatcagcggcctgaaaacagaggacgaggccgactactactgccagagctacgactacgacagcagcaacgtgctgtttggaggcggcaccaagctgacagtgctt

SEQ ID NO: 266 - DNA, 750 bp, Artificial Sequence
  GPRC5D-204 VL/VH (nt)
aacttcatgctgacccagcctcacagcgtgtccgagtctccaggcaagaccgtgtccatcagctgcaccagaacaagcggagccattgccggcgcttacgtgcagtggttccagcagaggcctggaagcgctcctaccaccgtgatctacgacgacaacaagaggcctagcggcgtgcccgatagattcagcggcagcatcgacaagagcagcaacagcgccagcctgacaatcagcggcctgaaaacagaggacgaggccgactactactgccagagctacgactacgacagcagcaacgtgctgtttggcggaggcaccaagctgacagtgcttggaagtagaggcggcggaggaagcggaggcggaggatctggtggtggtggatctctggaaatggccgaagtgcagctggtcgagtctggcggaggacttgttcaacctggcggcagcctgagactgtcttgtgccgcttccggcttcaccttcagcaactacgccatgtcctgggtccgacaggcccctggaaaaggactggaatgggtgtccgccatcagcggctctggcaatacctactacgccgacagcgtgaagggcagattcaccatcagccgggacaacagcaagaacaccctgtacctgcagatgaactccctgagagccgaggacaccgccgtgtactattgtgccagaggcagcgtgcggtacaccgatatttggggccagggcacactggtcaccgtgtcatct

SEQ ID NO: 267 - DNA, 750 bp, Artificial Sequence
  GPRC5D-205 VH/VL (nt)
gaagtgcagctggttgaatctggcggcggactgattcagcctggcggatctctgagactgagctgtgccgccagcggcttcaccttcagcaactacgccatgaactgggtccgacaggcccctggcaaaggccttgaatgggtgtccaccatcaacggcagaggcagcagcaccatctacgccgattctgtgaagggcagattcaccatcagccgggacaacagcaagaacaccctgtacctgcagatgaacagcctgagagccgaggacaccgccacctattactgtgccagatatatcagcagaggcctgggcgattcttggggccagggaacactggtcacagtgtctagtggaagtagaggcggcggaggatctggcggaggtggaagtggcggaggcggttctcttgaaatggcccagtctgtggtcacccagccacctagcatgtctgccgctccaggacagcaagtgaccatctcttgtagcggcggcaacagcaacatcgagcggaactacgtgtcctggtatctgcagctgcctggcacagcccctaagctggtcatcttcgacaacgacagacggcccagcggcatccccgatagattttctggcagcaagagcggcaccagcgccacactgggaattacaggactgcagacaggcgacgaggccgactactattgtggcacctgggacagctccctgagaggctgggttttcggaggcggcacaaagctgacagtgctc

SEQ ID NO: 268 - DNA, 750 bp, Artificial Sequence
  GPRC5D-205 VL/VH (nt)
cagtctgtggttacacagcctcctagcatgtctgccgctcccgggcagcaagtgaccatctcttgtagcggcggcaacagcaacatcgagcggaactacgtgtcctggtatctgcagctgcctggcacagcccctaagctggtcatcttcgacaacgacagacggcccagcggcatccccgatagattttctggcagcaagagcggcaccagcgccacactgggaattacaggactgcagacaggcgacgaggccgactactattgtggcacctgggactctagcctgagaggctgggttttcggcggaggcacaaagctgacagtgctgggaagtagaggcggcggaggatctggcggaggtggaagtggcggaggcggatctcttgaaatggccgaggtgcagctggtggaatcaggcggaggactgattcagcctggcggctctctgagactgtcttgtgccgccagcggcttcaccttcagcaactacgccatgaactgggtccgacaggcccctggcaaaggccttgaatgggtgtccaccatcaacggcagaggcagcagcaccatctacgccgattctgtgaagggcagattcaccatcagccgggacaacagcaagaacaccctgtacctgcagatgaactccctgagagccgaggacaccgccacctattactgtgccagatatatcagcagaggcctgggcgattcttggggccagggaacactggtcacagtctctagt

SEQ ID NO: 269 - DNA, 759 bp, Artificial Sequence
  GPRC5D-206 VH/VL (nt)
caagttcagctggttcagtctggcgccgaagtgaagaaacctggcgcctctgtgaaggtgtcctgcaaggccagcggctacacctttaccagctactacatgcactgggtccgacaggcccctggacaaggacttgagtggatgggcatcatcaaccctagcggcggcagcacaagatacgcccagaaattccagggcagagtgaccatgaccagagacaccagcacctccaccgtgtatatggaactgagcagcctgcggagcgaggacacagccgtgtactattgtgccagaggcagcagcagatggggcggatggacaggcgattattggggccagggaaccctggtcacagtgtctagtggaagtagaggcggcggaggatctggcggaggtggaagtggcggaggcggatctcttgaaatggcccagtctgccctgacacagcctgcctctgtttctggctctcctggccagagcatcaccatcagctgtaccggcaccagctctgatgtcggcggctacaatttcgtgtcctggtatcagcagcaccccggcaaggcccctaaagtgatgatctacgacgtgtccaagcggcccagcggcatcagcaatagattcagcggcagcaagagcggcaacaccgccagcctgacaattagcggactgcaggttgaggacgaggccgagtactactgcagcagctacacaagcaccagaaccgtgatcttcgctggcggcaccaaagtgacagtgctc

SEQ ID NO: 270 - DNA, 759 bp, Artificial Sequence
  GPRC5D-206 VL/VH (nt)
caatctgctctgacacagcctgccagcgtgtccggatctcctggccagagcatcaccatcagctgtaccggcaccagctctgatgtcggcggctacaatttcgtgtcctggtatcagcagcaccccggcaaggcccctaaagtgatgatctacgacgtgtccaagcggcccagcggcatcagcaatagattcagcggcagcaagagcggcaacaccgccagcctgacaattagcggactgcaggttgaggacgaggccgagtactactgcagcagctacaccagcaccagaaccgtgatctttgctggcggcaccaaagtgacagtgctgggaagtagaggcggcggaggatctggcggaggtggaagtggcggaggcggatctcttgaaatggctcaggtgcagctggtgcagtctggcgccgaagtgaaaaaacctggcgcctccgtgaaggtgtcctgcaaggcttctggctacacctttaccagctactacatgcactgggtccgacaggcccctggacaaggacttgagtggatgggcatcatcaaccctagcggcggcagcacaagatacgcccagaaattccagggcagagtgaccatgaccagagacacctccaccagcacagtgtatatggaactgagcagcctgcggagcgaggacacagccgtgtactattgtgccagaggcagcagcagatggggcggatggacaggcgattattggggccagggaaccctggtcacagtgtctagc

SEQ ID NO: 271 - DNA, 60 bp, Homo sapiens
  human IgGkappa signal sequence (nt)
atggtgctgcagacccaggtgttcatcagcctgctgctgtggatctccggagcatacgga

SEQ ID NO: 272 - protein, 20 aa, Homo sapiens
  human IgGkappa signal peptide(aa)
MVLQTQVFISLLLWISGAYG

SEQ ID NO: 273 - DNA, 60 bp, Homo sapiens
  human IgGkappa signal sequence (nt)
atggtgctgcagacccaggtgttcatcagcctgctgctgtggatctctggcgcctacggc

SEQ ID NO: 274 - DNA, 60 bp, Homo sapiens
  human IgGkappa signal sequence (nt)
atggtgctgcagacccaggtgttcatcagcctgctgctgtggatctctggcgcctatgga

SEQ ID NO: 275 - DNA, 60 bp, Homo sapiens
  human IgGkappa signal sequence (nt)
atggtgctgcagacacaggtgttcatctccctgctgctgtggatctctggagcatacgga

SEQ ID NO: 276 - DNA, 60 bp, Homo sapiens
  human IgGkappa signal sequence (nt)
atggtgctgcagacacaggtgttcatcagcctgctgctgtggatctccggagcatacgga

SEQ ID NO: 277 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 278 - protein, 22 aa, Artificial Sequence
  GMCSFR alpha chain signal peptide
MLLLVTSLLLCELPHPAFLLIP

SEQ ID NO: 279 - protein, 18 aa, Artificial Sequence
  CD8 alpha signal peptide
MALPVTALLLPLALLLHA

SEQ ID NO: 280 - protein, 16 aa, Artificial Sequence
  CD33 signal peptide
MPLLLLLPLLWAGALA

SEQ ID NO: 281 - protein, 327 aa, Homo sapiens
  Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 282 - protein, 326 aa, Homo sapiens
  Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 283 - DNA, 684 bp, Artificial Sequence
  IgG4/IgG2 hinge- IgG2/IgG4 CH2- IgG4 CH3 spacer codon optimized only (nt)
gagtctaaatacggaccgccttgtcctccttgtccagctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaaa

SEQ ID NO: 284 - DNA, 684 bp, Artificial Sequence
  Alternative CO/SSE IgG4/IgG2 hinge- IgG2/IgG4 CH2-IgG4 CH3 (nt)
gaatctaagtacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaag

SEQ ID NO: 285 - DNA, 36 bp, Artificial Sequence
  Spacer (IgG4hinge) (nt)
gaatctaagtacggaccgccctgccccccttgccct

SEQ ID NO: 286 - protein, 229 aa, Artificial Sequence
  Hinge-CH2-CH3 spacer (aa)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 287 - DNA, 117 bp, Artificial Sequence
  CD28 ectodomain spacer (nt)
attgaagttatgtatcctcctccttacctagacaatgagaagagcaatggaaccattatccatgtgaaagggaaacacctttgtccaagtcccctatttcccggaccttctaagccc

SEQ ID NO: 288 - protein, 39 aa, Artificial Sequence
  CD28 ectodomain spacer (aa)
IEVMYPPPYLDNEKSNGTIIHVKGKHLCPSPLFPGPSKP

SEQ ID NO: 289 - protein, 2028 aa, Artificial Sequence
  anti-GPRC5D CAR
ATGCCGCTGCTGCTACTGCTGCCCCTGCTGTGGGCAGGGGCTCTAGCTTCTTCTGAGCTGACCCAAGATCCTGCCGTGTCTGTGGCTCTGGGCCAGACAGTGCGGATTACCTGTCAGGGCGATAGCCTGAGAAGCTACTACGCCAGCTGGTATCAGCAGAAGCCTGGACAGGCTCCCGTGCTGGTCATCTACGGCAAGAACAACAGACCCAGCGGCATCCCCGATAGATTCAGCGGAAGCAGCTCTGGCAATACCGCCAGCCTGACAATTACTGGCGCCCAGGCCGAAGATGAGGCCGACTACTACTGCAACAGCAGAGACAGCTCCGGCAATCCTCCTGTGGTTTTTGGCGGAGGCACCAAGCTGACAGTGCTGGGAAGTAGAGGTGGCGGAGGATCTGGCGGCGGAGGAAGCGGAGGCGGCGGATCTCTTGAAATGGCTCAGGTGCAGCTGGTGGAATCAGGCGGTGGACTTGTTCACCCTGGCGGAAGCCTGAGACTGTCTTGTGCCGCCAGCGGCTTCACCTTCAGATCCCACAGCATGAACTGGGTCCGACAGGCCCCTGGCAAAGGCCTTGAATGGGTGTCCAGCATCAGCAGCGACAGCACCTACACCTACTATGCCGACAGCGTGAAGGGCAGATTCACCATCTCCAGAGACAACGCCAAGAACAGCCTGTACCTGCAGATGAACTCCCTGAGAGCCGAGGACACCGCCGTGTACTACTGTGCTAGAAGTGGCGGCCAGTGGAAGTACTACGACTATTGGGGCCAGGGCACCCTGGTCACAGTTAGCTCTGAGTCTAAATACGGACCGCCTTGTCCTCCTTGTCCCGCTCCTCCTGTTGCCGGACCTTCCGTGTTCCTGTTTCCTCCAAAGCCTAAGGACACCCTGATGATCAGCAGGACCCCTGAAGTGACCTGCGTGGTGGTGGATGTGTCCCAAGAGGATCCCGAGGTGCAGTTCAACTGGTATGTGGACGGCGTGGAAGTGCACAACGCCAAGACCAAGCCTAGAGAGGAACAGTTCCAGAGCACCTACAGAGTGGTGTCCGTGCTGACAGTGCTGCACCAGGATTGGCTGAACGGCAAAGAGTACAAGTGCAAGGTGTCCAACAAGGGCCTGCCTAGCAGCATCGAGAAAACCATCTCCAAGGCCAAGGGCCAGCCAAGAGAGCCCCAGGTTTACACACTGCCTCCAAGCCAAGAGGAAATGACCAAGAATCAGGTGTCCCTGACATGCCTGGTCAAGGGCTTCTACCCCTCCGATATCGCCGTGGAATGGGAGAGCAATGGCCAGCCTGAGAACAACTACAAGACCACACCTCCTGTGCTGGACAGCGACGGCAGTTTCTTCCTGTATAGTAGACTCACCGTGGATAAATCAAGATGGCAAGAGGGCAACGTGTTCAGCTGCAGCGTGATGCACGAGGCCCTGCACAACCACTACACCCAGAAAAGCCTGAGCCTGTCTCTGGGCAAGATGTTCTGGGTGCTCGTGGTCGTTGGCGGAGTGCTGGCCTGTTACAGCCTGCTGGTTACCGTGGCCTTCATCATCTTTTGGGTCAAGCGGGGCAGAAAGAAGCTGCTCTACATCTTCAAGCAGCCCTTCATGCGGCCCGTGCAGACCACACAAGAGGAAGATGGCTGCTCCTGCAGATTCCCCGAGGAAGAAGAAGGCGGCTGCGAGCTGAGAGTGAAGTTCAGCAGATCCGCCGACGCTCCAGCCTATCAGCAGGGCCAAAACCAGCTGTACAACGAGCTGAACCTGGGGAGAAGAGAAGAGTACGACGTGCTGGATAAGCGGAGAGGCAGAGATCCTGAAATGGGCGGCAAGCCCAGACGGAAGAATCCTCAAGAGGGCCTGTATAATGAGCTGCAGAAAGACAAGATGGCCGAGGCCTACAGCGAGATCGGAATGAAGGGCGAGCGCAGAAGAGGCAAGGGACACGATGGACTGTACCAGGGCCTGAGCACCGCCACCAAGGATACCTATGACGCACTGCACATGCAGGCCCTGCCACCTAGA

SEQ ID NO: 290 - protein, 660 aa, Artificial Sequence; identical to SEQ ID NO: 72
  anti-GPRC5D CAR

SEQ ID NO: 291 - DNA, 20 bp, Artificial Sequence
  BCMA sgRNA Target Sequence
caatggtcagagtcgacctt

SEQ ID NO: 292 - RNA, 20 nt, Artificial Sequence
  GPRC5D exon 1 gRNA1
ggguguggaccucaccagaa

SEQ ID NO: 293 - RNA, 20 nt, Artificial Sequence
  GPRC5D exon 1 gRNA2
gagaagaaaauagucuccag

SEQ ID NO: 294 - RNA, 20 nt, Artificial Sequence
  GPRC5D exon 1 gRNA3
gugacucucaucaugaccag

SEQ ID NO: 295 - RNA, 20 nt, Artificial Sequence
  GPRC5D exon 1 gRNA4
gacgaccgggucgucccacu

SEQ ID NO: 296 - RNA, 20 nt, Artificial Sequence
  GPRC5D exon 1 gRNA5
gagaaccaggagcucuccag

SEQ ID NO: 297 - protein, 13 aa, Artificial Sequence
  CDRL3
SSYTSTRTVIFAG

SEQ ID NO: 298 - protein, 1374 aa, Artificial Sequence
  BCMA-GPRC5D CAR (aa)
MVLQTQVFISLLLWISGAYGQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMPLLLLLPLLWAGALASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 299 - DNA, 4122 bp, Artificial Sequence
  BCMA-GPRC5D CAR codon diverged (nt)
atggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagacttgaaggtggtggcgaaggcagaggcagcctgcttacatgcggagatgtggaagagaaccccggacctagaatgccgctgctgctactgctgcccctgctgtgggcaggggctctagcttcttctgagctgacccaagatcctgccgtgtctgtggctctgggccagacagtgcggattacctgtcagggcgatagcctgagaagctactacgccagctggtatcagcagaagcctggacaggctcccgtgctggtcatctacggcaagaacaacagacccagcggcatccccgatagattcagcggaagcagctctggcaataccgcctccctgacaattactggcgcccaggccgaagatgaggccgactactactgcaacagcagagacagctccggcaatcctcctgtggtgtttggaggcggaaccaaactgaccgtgctgggcagcagaggtggaggtggaagcggcggtggaggctccggaggaggcggaagccttgagatggcacaggtgcagctggtggaatcaggcggtggacttgttcaccctggcggaagcctgagactgtcttgtgccgccagcggcttcaccttccggtcccacagcatgaactgggtccgacaggcccctggcaaaggccttgaatgggtgtccagcatcagcagcgacagcacctacacctactatgccgacagcgtgaagggcagattcaccatctccagagacaacgccaagaacagcctgtacctgcagatgaactccctgagagccgaggacaccgccgtgtactactgtgctagaagtggcggccagtggaagtactacgactattggggacagggcaccctggtcacagttagctctgaatccaaatacggtcctccatgccctccatgcccagctccacccgttgctggaccaagcgtgtttctgttcccaccaaagcccaaggatacactcatgattagcagaaccccagaagtgacatgtgtcgtcgtggacgtttcccaagaggaccctgaggtccagtttaactggtatgtggatggagtggaggtgcataatgctaagactaagccaagagaagagcagtttcagtccacctatagagtggtctccgtgctcaccgtgctgcatcaggactggctcaatggcaaggaatataagtgtaaagtctccaacaaaggactgccatccagcatcgaaaagaccattagcaaggccaaaggacagcctagagagcctcaggtctacactctgcctccctcccaagaagagatgactaagaaccaggtctctctgacctgcctggtgaagggattctacccttccgacattgctgtggagtgggagtccaatggacagccagagaataactataagactacaccacccgtgctcgatagcgacggctcattctttctgtactctcggctgacagtggacaagagcagatggcaggagggaaacgtctttagctgctccgtgatgcatgaggctctgcataaccactatacccagaagtctctgtccctgagcctcggcaaaatgttttgggtgctggtggtggtcggaggcgtgctggcttgctattccctgctggtcacagtggccttcattatcttctgggtgaaacggggaagaaagaaactcctgtatatattcaaacaaccatttatgagaccagtacaaactactcaagaagaggatggctgtagctgccggtttcccgaagaagaggaaggaggatgtgaactgcgggtgaagttctccagaagcgccgacgcccctgcctaccagcagggacagaatcagctgtataacgaactgaacctgggcagaagggaggaatacgacgtcctggacaagagacgaggccgggaccccgagatgggcggaaagcctcggcggaagaacccccaggaaggcctgtacaacgaactgcagaaggacaagatggctgaagcttactccgagatcggcatgaaaggagagcggaggcggggcaagggccacgacggcctgtatcagggcctgtccaccgccacaaaggatacctacgatgccctccatatgcaggctctgcctccaaga

SEQ ID NO: 300 - DNA, 4122 bp, Artificial Sequence
  BCMA-GPRC5D CAR original (nt)
atggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagacttgaaggtggtggcgaaggcagaggcagcctgcttacatgcggagatgtggaagagaaccccggacctagaatgccgctgctgctactgctgcccctgctgtgggcaggggctctagcttcttctgagctgacccaagatcctgccgtgtctgtggctctgggccagacagtgcggattacctgtcagggcgatagcctgagaagctactacgccagctggtatcagcagaagcctggacaggctcccgtgctggtcatctacggcaagaacaacagacccagcggcatccccgatagattcagcggaagcagctctggcaataccgccagcctgacaattactggcgcccaggccgaagatgaggccgactactactgcaacagcagagacagctccggcaatcctcctgtggtttttggcggaggcaccaagctgacagtgctgggaagtagaggtggcggaggatctggcggcggaggaagcggaggcggcggatctcttgaaatggctcaggtgcagctggtggaatcaggcggtggacttgttcaccctggcggaagcctgagactgtcttgtgccgccagcggcttcaccttcagatcccacagcatgaactgggtccgacaggcccctggcaaaggccttgaatgggtgtccagcatcagcagcgacagcacctacacctactatgccgacagcgtgaagggcagattcaccatctccagagacaacgccaagaacagcctgtacctgcagatgaactccctgagagccgaggacaccgccgtgtactactgtgctagaagtggcggccagtggaagtactacgactattggggccagggcaccctggtcacagttagctctgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 301 - protein, 1374 aa, Artificial Sequence
  GPRC5D-BCMA CAR (aa)
MPLLLLLPLLWAGALASSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMVLQTQVFISLLLWISGAYGQSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 302 - DNA, 4122 bp, Artificial Sequence
  GPRC5D-BCMA CAR codon diverged (nt)
atgccgctgctgctactgctgcccctgctgtgggcaggggctctagcttcttctgagctgacccaagatcctgccgtgtctgtggctctgggccagacagtgcggattacctgtcagggcgatagcctgagaagctactacgccagctggtatcagcagaagcctggacaggctcccgtgctggtcatctacggcaagaacaacagacccagcggcatccccgatagattcagcggaagcagctctggcaataccgcctccctgacaattactggcgcccaggccgaagatgaggccgactactactgcaacagcagagacagctccggcaatcctcctgtggtgtttggaggcggaaccaaactgaccgtgctgggcagcagaggtggaggtggaagcggcggtggaggctccggaggaggcggaagccttgagatggcacaggtgcagctggtggaatcaggcggtggacttgttcaccctggcggaagcctgagactgtcttgtgccgccagcggcttcaccttccggtcccacagcatgaactgggtccgacaggcccctggcaaaggccttgaatgggtgtccagcatcagcagcgacagcacctacacctactatgccgacagcgtgaagggcagattcaccatctccagagacaacgccaagaacagcctgtacctgcagatgaactccctgagagccgaggacaccgccgtgtactactgtgctagaagtggcggccagtggaagtactacgactattggggacagggcaccctggtcacagttagctctgaatccaaatacggtcctccatgccctccatgcccagctccacccgttgctggaccaagcgtgtttctgttcccaccaaagcccaaggatacactcatgattagcagaaccccagaagtgacatgtgtcgtcgtggacgtttcccaagaggaccctgaggtccagtttaactggtatgtggatggagtggaggtgcataatgctaagactaagccaagagaagagcagtttcagtccacctatagagtggtctccgtgctcaccgtgctgcatcaggactggctcaatggcaaggaatataagtgtaaagtctccaacaaaggactgccatccagcatcgaaaagaccattagcaaggccaaaggacagcctagagagcctcaggtctacactctgcctccctcccaagaagagatgactaagaaccaggtctctctgacctgcctggtgaagggattctacccttccgacattgctgtggagtgggagtccaatggacagccagagaataactataagactacaccacccgtgctcgatagcgacggctcattctttctgtactctcggctgacagtggacaagagcagatggcaggagggaaacgtctttagctgctccgtgatgcatgaggctctgcataaccactatacccagaagtctctgtccctgagcctcggcaaaatgttttgggtgctggtggtggtcggaggcgtgctggcttgctattccctgctggtcacagtggccttcattatcttctgggtgaaacggggaagaaagaaactcctgtatatattcaaacaaccatttatgagaccagtacaaactactcaagaagaggatggctgtagctgccggtttcccgaagaagaggaaggaggatgtgaactgcgggtgaagttctccagaagcgccgacgcccctgcctaccagcagggacagaatcagctgtataacgaactgaacctgggcagaagggaggaatacgacgtcctggacaagagacgaggccgggaccccgagatgggcggaaagcctcggcggaagaacccccaggaaggcctgtacaacgaactgcagaaggacaagatggctgaagcttactccgagatcggcatgaaaggagagcggaggcggggcaagggccacgacggcctgtatcagggcctgtccaccgccacaaaggatacctacgatgccctccatatgcaggctctgcctccaagactcgagggcggcggagagggcagaggaagtcttctaacatgcggtgacgtggaggagaatcccggccctaggatggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 303 - DNA, 4122 bp, Artificial Sequence
  GPRC5D-BCMA CAR original (nt)
atgccgctgctgctactgctgcccctgctgtgggcaggggctctagcttcttctgagctgacccaagatcctgccgtgtctgtggctctgggccagacagtgcggattacctgtcagggcgatagcctgagaagctactacgccagctggtatcagcagaagcctggacaggctcccgtgctggtcatctacggcaagaacaacagacccagcggcatccccgatagattcagcggaagcagctctggcaataccgccagcctgacaattactggcgcccaggccgaagatgaggccgactactactgcaacagcagagacagctccggcaatcctcctgtggtttttggcggaggcaccaagctgacagtgctgggaagtagaggtggcggaggatctggcggcggaggaagcggaggcggcggatctcttgaaatggctcaggtgcagctggtggaatcaggcggtggacttgttcaccctggcggaagcctgagactgtcttgtgccgccagcggcttcaccttcagatcccacagcatgaactgggtccgacaggcccctggcaaaggccttgaatgggtgtccagcatcagcagcgacagcacctacacctactatgccgacagcgtgaagggcagattcaccatctccagagacaacgccaagaacagcctgtacctgcagatgaactccctgagagccgaggacaccgccgtgtactactgtgctagaagtggcggccagtggaagtactacgactattggggccagggcaccctggtcacagttagctctgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagactcgagggcggcggagagggcagaggaagtcttctaacatgcggtgacgtggaggagaatcccggccctaggatggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 304 - DNA, 48 bp, Homo sapiens
  huCD33 signal peptide (nt)
atgccgctgctgctactgctgcccctgctgtgggcaggggctctagct

SEQ ID NO: 305 - DNA, 684 bp, Artificial Sequence
  Long spacer codon diverged (nt)
gaatccaaatacggtcctccatgccctccatgcccagctccacccgttgctggaccaagcgtgtttctgttcccaccaaagcccaaggatacactcatgattagcagaaccccagaagtgacatgtgtcgtcgtggacgtttcccaagaggaccctgaggtccagtttaactggtatgtggatggagtggaggtgcataatgctaagactaagccaagagaagagcagtttcagtccacctatagagtggtctccgtgctcaccgtgctgcatcaggactggctcaatggcaaggaatataagtgtaaagtctccaacaaaggactgccatccagcatcgaaaagaccattagcaaggccaaaggacagcctagagagcctcaggtctacactctgcctccctcccaagaagagatgactaagaaccaggtctctctgacctgcctggtgaagggattctacccttccgacattgctgtggagtgggagtccaatggacagccagagaataactataagactacaccacccgtgctcgatagcgacggctcattctttctgtactctcggctgacagtggacaagagcagatggcaggagggaaacgtctttagctgctccgtgatgcatgaggctctgcataaccactatacccagaagtctctgtccctgagcctcggcaaa

SEQ ID NO: 306 - protein, 184 aa, Homo sapiens
  BCMA protein (Uniprot Q02223)
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 307 - DNA, 84 bp, Homo sapiens
  huCD28 transmembrane domain codon diverged (nt)
atgttttgggtgctggtggtggtcggaggcgtgctggcttgctattccctgctggtcacagtggccttcattatcttctgggtg

SEQ ID NO: 308 - DNA, 126 bp, Artificial Sequence
  4-1BB endodomain codon diverged (nt)
aaacggggaagaaagaaactcctgtatatattcaaacaaccatttatgagaccagtacaaactactcaagaagaggatggctgtagctgccggtttcccgaagaagaggaaggaggatgtgaactg

SEQ ID NO: 309 - DNA, 336 bp, Artificial Sequence
  CD3zeta endodomain codon diverged (nt)
cgggtgaagttctccagaagcgccgacgcccctgcctaccagcagggacagaatcagctgtataacgaactgaacctgggcagaagggaggaatacgacgtcctggacaagagacgaggccgggaccccgagatgggcggaaagcctcggcggaagaacccccaggaaggcctgtacaacgaactgcagaaggacaagatggctgaagcttactccgagatcggcatgaaaggagagcggaggcggggcaagggccacgacggcctgtatcagggcctgtccaccgccacaaaggatacctacgatgccctccatatgcaggctctgcctccaaga

SEQ ID NO: 310 - DNA, 732 bp, Artificial Sequence
  BCMA-55 scFv (nt)
cagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagc

SEQ ID NO: 311 - DNA, 750 bp, Artificial Sequence
  GPRC5D scFv codon diverged (nt)
tcttctgagctgacccaagatcctgccgtgtctgtggctctgggccagacagtgcggattacctgtcagggcgatagcctgagaagctactacgccagctggtatcagcagaagcctggacaggctcccgtgctggtcatctacggcaagaacaacagacccagcggcatccccgatagattcagcggaagcagctctggcaataccgcctccctgacaattactggcgcccaggccgaagatgaggccgactactactgcaacagcagagacagctccggcaatcctcctgtggtgtttggaggcggaaccaaactgaccgtgctgggcagcagaggtggaggtggaagcggcggtggaggctccggaggaggcggaagccttgagatggcacaggtgcagctggtggaatcaggcggtggacttgttcaccctggcggaagcctgagactgtcttgtgccgccagcggcttcaccttccggtcccacagcatgaactgggtccgacaggcccctggcaaaggccttgaatgggtgtccagcatcagcagcgacagcacctacacctactatgccgacagcgtgaagggcagattcaccatctccagagacaacgccaagaacagcctgtacctgcagatgaactccctgagagccgaggacaccgccgtgtactactgtgctagaagtggcggccagtggaagtactacgactattggggacagggcaccctggtcacagttagctct

SEQ ID NO: 312 - DNA, 2399 bp, Artificial Sequence
  BCMA-41BB CAR
ccatatgagatcttatatggggcacccccgccccttgtaaacttccctgaccctgacataacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 313 - DNA, 2028 bp, Artificial Sequence; identical to SEQ ID NO: 70
  GPRC5D-41BB CAR

SEQ ID NO: 314 - DNA, 2025 bp, Artificial Sequence
  GPRC5D-CD28 CAR
atgccgctgctgctactgctgcccctgctgtgggcaggggctctagcttcttctgagctgacccaagatcctgccgtgtctgtggctctgggccagacagtgcggattacctgtcagggcgatagcctgagaagctactacgccagctggtatcagcagaagcctggacaggctcccgtgctggtcatctacggcaagaacaacagacccagcggcatccccgatagattcagcggaagcagctctggcaataccgccagcctgacaattactggcgcccaggccgaagatgaggccgactactactgcaacagcagagacagctccggcaatcctcctgtggtttttggcggaggcaccaagctgacagtgctgggaagtagaggtggcggaggatctggcggcggaggaagcggaggcggcggatctcttgaaatggctcaggtgcagctggtggaatcaggcggtggacttgttcaccctggcggaagcctgagactgtcttgtgccgccagcggcttcaccttcagatcccacagcatgaactgggtccgacaggcccctggcaaaggccttgaatgggtgtccagcatcagcagcgacagcacctacacctactatgccgacagcgtgaagggcagattcaccatctccagagacaacgccaagaacagcctgtacctgcagatgaactccctgagagccgaggacaccgccgtgtactactgtgctagaagtggcggccagtggaagtactacgactattggggccagggcaccctggtcacagttagctctgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaggagtaagaggagcaggctcctgcacagtgactacatgaacatgactccccgccgccccgggcccacccgcaagcattaccagccctatgccccaccacgcgacttcgcagcctatcgctccagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 315 - DNA, 4122 bp, Artificial Sequence
  BCMA-41BB-GPRC5D-41BB dual CAR
atggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagactcgagggcggcggagagggaaggggatccctcctgacctgtggcgacgtcgaggaaaatcctggccccaggatgccgctgctgctactgctgcccctgctgtgggcaggggctctagcttcttctgagctgacccaagatcctgccgtgtctgtggctctgggccagacagtgcggattacctgtcagggcgatagcctgagaagctactacgccagctggtaccaacaaaagcccggacaggctcctgtgctcgtcatttatggcaagaacaacagaccttccggaatccccgataggttttccggaagctcctctggaaatacagcttccctcaccatcactggcgcccaagctgaagatgaagctgactattattgtaactccagagacagctccggcaatcctcctgtcgtgttcggaggcggaacaaaattgaccgtgctcggcagcaggggcggaggcggctctggaggtggcgggtccggcggaggaggcagcctggagatggcccaggtccaactcgtcgaatcaggcggtggacttgttcatcccggcggaagcctgaggctctcttgtgccgcttccggattcacttttagatcccacagcatgaactgggtccgacaagctcccggcaaaggccttgaatgggtgtccagcatcagcagcgacagcacctacacctactatgccgacagcgtgaaaggaaggttcacaatctctagggacaacgccaagaacagcctgtacctgcagatgaactccctcagggctgaggatacagctgtctattattgtgctagaagtggcggccagtggaagtactacgactactggggacaaggcacactcgtgacagttagctctgaaagcaagtatggccccccctgcccaccctgccctgccccacccgtggctggccccagcgtctttctcttcccccctaaacccaaagatacactcatgatttccagaacacccgaggtcacatgtgtcgtcgtcgacgtcagccaggaagaccctgaagtccaatttaattggtacgtcgatggagtcgaggtccataatgctaaaacaaaacccagggaagagcaatttcaatccacatatagggtcgtcagcgtcctcaccgtcctccatcaagactggctcaatggaaaggaatataaatgtaaagtcagcaataaaggactcccctcctccattgaaaagacaattagcaaagctaaaggacaacccagggaacctcaagtgtataccctgcctcccagccaggaagagatgacaaaaaaccaagtcagcctcacctgtctcgtgaaaggattttatcctagcgacattgctgtcgagtgggaatccaacggacaacccgaaaataattataaaacaaccccccccgtcctcgattccgatggaagcttttttctctacagcaggctgacagtcgacaagagcaggtggcaggaaggaaatgtcttttcctgttccgtcatgcatgaagctctccataatcattatacacaaaagtccctctccctcagcctcggaaaaatgttttgggtcctggtcgtggtgggaggcgtcctcgcttgctattccctcctcgtgacagtcgcctttattatcttctgggtgaaaagaggaaggaaaaaactcctgtatatttttaaacaaccttttatgagacctgtccaaacaacccaggaagaggacggatgtagctgtaggtttcctgaagaggaggagggaggatgtgaactcagggtcaaattttccaggagcgctgatgcccccgcttaccaacaaggacagaatcaactctataatgaactcaatctcggcaggagggaggaatatgatgtcctcgacaaaagaaggggaagggaccccgagatgggaggaaaacctaggagaaaaaacccccaggaaggactctacaacgaactccaaaaggataaaatggctgaagcttattccgaaattggcatgaaaggagaaagaaggaggggaaaaggccatgacggcctctatcaaggactctccacagctacaaaagacacatacgatgccctccatatgcaagctctcccccccagg

SEQ ID NO: 316 - DNA, 4119 bp, Artificial Sequence
  BCMA-41BB-GPRC5D-CD28 dual CAR
atggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctagactcgagggcggcggagagggaaggggatccctcctgacctgtggcgacgtcgaggaaaatcctggccccaggatgccgctgctgctactgctgcccctgctgtgggcaggggctctagcttcttctgagctgacccaagatcctgccgtgtctgtggctctgggccagacagtgcggattacctgtcagggcgatagcctgagaagctactacgccagctggtaccaacaaaagcccggacaggctcctgtgctcgtcatttatggcaagaacaacagaccttccggaatccccgataggttttccggaagctcctctggaaatacagcttccctcaccatcactggcgcccaagctgaagatgaagctgactattattgtaactccagagacagctccggcaatcctcctgtcgtgttcggaggcggaacaaaattgaccgtgctcggcagcaggggcggaggcggctctggaggtggcgggtccggcggaggaggcagcctggagatggcccaggtccaactcgtcgaatcaggcggtggacttgttcatcccggcggaagcctgaggctctcttgtgccgcttccggattcacttttagatcccacagcatgaactgggtccgacaagctcccggcaaaggccttgaatgggtgtccagcatcagcagcgacagcacctacacctactatgccgacagcgtgaaaggaaggttcacaatctctagggacaacgccaagaacagcctgtacctgcagatgaactccctcagggctgaggatacagctgtctattattgtgctagaagtggcggccagtggaagtactacgactactggggacaaggcacactcgtgacagttagctctgaaagcaagtatggccccccctgcccaccctgccctgccccacccgtggctggccccagcgtctttctcttcccccctaaacccaaagatacactcatgatttccagaacacccgaggtcacatgtgtcgtcgtcgacgtcagccaggaagaccctgaagtccaatttaattggtacgtcgatggagtcgaggtccataatgctaaaacaaaacccagggaagagcaatttcaatccacatatagggtcgtcagcgtcctcaccgtcctccatcaagactggctcaatggaaaggaatataaatgtaaagtcagcaataaaggactcccctcctccattgaaaagacaattagcaaagctaaaggacaacccagggaacctcaagtgtataccctgcctcccagccaggaagagatgacaaaaaaccaagtcagcctcacctgtctcgtgaaaggattttatcctagcgacattgctgtcgagtgggaatccaacggacaacccgaaaataattataaaacaaccccccccgtcctcgattccgatggaagcttttttctctacagcaggctgacagtcgacaagagcaggtggcaggaaggaaatgtcttttcctgttccgtcatgcatgaagctctccataatcattatacacaaaagtccctctccctcagcctcggaaaaatgttttgggtcctggtcgtggtgggaggcgtcctcgcttgctattccctcctcgtgacagtcgcctttattatcttctgggtgaggagtaagaggagcaggctcctgcacagtgactacatgaacatgactccccgccgccccgggcccacccgcaagcattaccagccctatgccccaccacgcgacttcgcagcctatcgctccagggtcaaattttccaggagcgctgatgcccccgcttaccaacaaggacagaatcaactctataatgaactcaatctcggcaggagggaggaatatgatgtcctcgacaaaagaaggggaagggaccccgagatgggaggaaaacctaggagaaaaaacccccaggaaggactctacaacgaactccaaaaggataaaatggctgaagcttattccgaaattggcatgaaaggagaaagaaggaggggaaaaggccatgacggcctctatcaaggactctccacagctacaaaagacacatacgatgccctccatatgcaagctctcccccccagg

SEQ ID NO: 317 - DNA, 2832 bp, Artificial Sequence
  GPRC5D-BCMA-41BB CAR
atggtgctgcagacccaggtgttcatcagcctgctgctgtggatctctggcgcctatggacaggtccaactcgtcgaatcaggcggtggacttgttcatcccggcggaagcctgaggctctcttgtgccgcttccggatttacatttagatcccacagcatgaactgggtccgacaagctcccggcaaaggccttgaatgggtgtccagcatcagcagcgacagcacctacacctactatgccgacagcgtgaaaggaaggttcacaatctctagggacaacgccaagaacagcctgtacctgcagatgaactccctcagggctgaggatacagctgtctattattgtgctagaagtggcggccagtggaagtactacgactactggggacaaggcacactcgtgacagttagctctggcagcaggggcggaggcggcagcggaggaggcggctccggcggaggaggcagcctggagatggcctcttctgagctgacccaagatcctgccgtgtctgtggctctgggccagacagtgcggattacctgtcagggcgatagcctgagaagctactacgccagctggtaccaacaaaagcccggacaggctcctgtgctcgtcatttatggcaagaacaacagaccttccggaatccccgataggttttccggaagctcctctggaaatacagcttccctcaccatcactggcgcccaagctgaagatgaagctgactattattgtaactccagagacagctccggcaatcctcctgtcgtgttcggaggcggaacaaaactcaccgtcctcggaggtggaggatctggaggcggtggaagcggagggggcggttccggtggcgggggatctcagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtcaagcggggcagaaagaagctgctctacatcttcaagcagcccttcatgcggcccgtgcagaccacacaagaggaagatggctgctcctgcagattccccgaggaagaagaaggcggctgcgagctgagagtgaagttcagcagatccgccgacgctccagcctatcagcagggccaaaaccagctgtacaacgagctgaacctggggagaagagaagagtacgacgtgctggataagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgacgcactgcacatgcaggccctgccacctaga

SEQ ID NO: 318 - DNA, 1560 bp, Artificial Sequence
  BCMA del CAR
atggtgctgcagacccaggtgttcatcagcctgctgctgtggatctctggcgcctatggacagtctgccctgacacagcctgccagcgttagtgctagtcccggacagtctatcgccatcagctgtaccggcaccagctctgacgttggctggtatcagcagcaccctggcaaggcccctaagctgatgatctacgaggacagcaagaggcccagcggcgtgtccaatagattcagcggcagcaagagcggcaacaccgccagcctgacaattagcggactgcaggccgaggacgaggccgattactactgcagcagcaacacccggtccagcacactggtttttggcggaggcaccaagctgacagtgctgggatctagaggtggcggaggatctggcggcggaggaagcggaggcggcggatctcttgaaatggctgaagtgcagctggtgcagtctggcgccgagatgaagaaacctggcgcctctctgaagctgagctgcaaggccagcggctacaccttcatcgactactacgtgtactggatgcggcaggcccctggacagggactcgaatctatgggctggatcaaccccaatagcggcggcaccaattacgcccagaaattccagggcagagtgaccatgaccagagacaccagcatcagcaccgcctacatggaactgagccggctgagatccgacgacaccgccatgtactactgcgccagatctcagcgcgacggctacatggattattggggccagggaaccctggtcaccgtgtccagcgagtctaaatacggaccgccttgtcctccttgtcccgctcctcctgttgccggaccttccgtgttcctgtttcctccaaagcctaaggacaccctgatgatcagcaggacccctgaagtgacctgcgtggtggtggatgtgtcccaagaggatcccgaggtgcagttcaactggtatgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatctccaaggccaagggccagccaagagagccccaggtttacacactgcctccaagccaagaggaaatgaccaagaatcaggtgtccctgacatgcctggtcaagggcttctacccctccgatatcgccgtggaatgggagagcaatggccagcctgagaacaactacaagaccacacctcctgtgctggacagcgacggcagtttcttcctgtatagtagactcaccgtggataaatcaagatggcaagagggcaacgtgttcagctgcagcgtgatgcacgaggccctgcacaaccactacacccagaaaagcctgagcctgtctctgggcaagatgttctgggtgctcgtggtcgttggcggagtgctggcctgttacagcctgctggttaccgtggccttcatcatcttttgggtc

SEQ ID NO: 319 - DNA, 72 bp, Artificial Sequence
  T2A
ctcgagggcggcggagagggaaggggatccctcctgacctgtggcgacgtcgaggaaaatcctggccccagg

SEQ ID NO: 320 - protein, 20 aa, Artificial Sequence
  linker
GGGGSGGGGSGGGGSGGGGS